(19) United States
(12) Patent Application Publication
(10) Pub. No.: US 2025/0388541 A1
(43) Pub. Date: Dec. 25, 2025

(54) HETEROARYL COMPOUNDS FOR THE TREATMENT OF PAIN

(71) Applicant: VERTEX PHARMACEUTICALS INCORPORATED, Boston, MA (US)

(72) Inventors: Mark Thomas MILLER, Rancho Santa Fe, CA (US); Carl Victor VOGEL, Carlsbad, CA (US); Thomas Aaron BEDELL, La Mesa, CA (US); Dennis James HURLEY, San Marcos, CA (US); Timothy Donald NEUBERT, San Diego, CA (US); Elamprakash NESAN SAVARIAR, San Diego, CA (US); Urvi PATEL, San Diego, CA (US); Sara Sabina HADIDA RUAH, La Jolla, CA (US); Jason McCARTNEY, Cardiff by the Sea, CA (US); Jinglan ZHOU, San Diego, CA (US); Robert Martin DEMORET, San Diego, CA (US); Roman Askatovich VALIULIN, San Diego, CA (US); Alexander Frederick KINTZER, Carlsbad, CA (US); Peter WEBB, San Diego, CA (US); David Robert SLOCHOWER, San Diego, CA (US); Kathleen AERTGEERTS, San Diego, CA (US); Elizabeth Mary BECK, Abingdon, Oxfordshire (GB); Ronald Marcellus Alphonsus KNEGTEL, Abingdon, Oxfordshire (GB); Ewa Iwona CHUDYK, Wantage, Oxfordshire (GB); Joanne Louise PINDER, Didcot, Oxfordshire (GB); Anton V. GULEVICH, San Diego, CA (US); Yoshihiro ISHIHARA, San Diego, CA (US); Joe A. TRAN, San Marcos, CA (US)

(73) Assignee: VERTEX PHARMACEUTICALS INCORPORATED, Boston, MA (US)

(21) Appl. No.: 18/858,142

(22) PCT Filed: Apr. 21, 2023
(86) PCT No.: PCT/US2023/019474

§ 371 Date: Oct. 18, 2024

Related U.S. Application Data
(60) Provisional application No. 63/333,814, filed on Apr. 22, 2022.

(51) Int. Cl. C07D 213/46; A61K 31/415; A61K 31/421; A61K 31/427; A61K 31/4365; A61K 31/437; A61K 31/444; A61K 31/4545; A61K 31/506; C07D 401/04; C07D 401/14; C07D 413/04; C07D 413/10; C07D 417/04; C07D 487/04; C07D 513/04

(52) U.S. Cl. CPC C07D 213/46; A61K 31/415; A61K 31/421; A61K 31/427; A61K 31/4365; A61K 31/437; A61K 31/444; A61K 31/4545; A61K 31/506; C07D 401/04; C07D 401/14; C07D 413/04; C07D 413/10; C07D 417/04; C07D 487/04; C07D 513/04

(57) ABSTRACT

Compounds, and pharmaceutically acceptable salts thereof, useful as inhibitors of sodium channels are provided. Also provided are pharmaceutical compositions comprising the compounds or pharmaceutically acceptable salts and methods of using the compounds, pharmaceutically acceptable salts, and pharmaceutical compositions in the treatment of various disorders, including pain.

### CROSS-REFERENCE TO RELATED APPLICATIONS

This application claims the benefit of U.S. Provisional Application No. 63/333,814, filed Apr. 22, 2022, which is incorporated by reference herein in its entirety.

### BACKGROUND

Pain is a protective mechanism that allows healthy animals to avoid tissue damage and to prevent further damage to injured tissue. Nonetheless, there are many conditions where pain persists beyond its usefulness, or where patients would benefit from inhibition of pain. Neuropathic pain is a form of chronic pain caused by an injury to the sensory nerves (Dieleman, J. P., et al., Incidence rates and treatment of neuropathic pain conditions in the general population. Pain, 2008. 137(3): p. 681-8). Neuropathic pain can be divided into two categories, pain caused by generalized metabolic damage to the nerve and pain caused by a discrete nerve injury. The metabolic neuropathies include post-herpetic neuropathy, diabetic neuropathy, and drug-induced neuropathy. Discrete nerve injury indications include post-amputation pain, post-surgical nerve injury pain, and nerve entrapment injuries like neuropathic back pain.

Voltage-gated sodium channels (Na_{V}s) are involved in pain signaling. Na_{V}s are biological mediators of electrical signaling as they mediate the rapid upstroke of the action potential of many excitable cell types (e.g. neurons, skeletal myocytes, cardiac myocytes). The evidence for the role of these channels in normal physiology, the pathological states arising from mutations in sodium channel genes, preclinical work in animal models, and the clinical pharmacology of known sodium channel modulating agents all point to the central role of Na_{V}s in pain sensation (Rush, A. M. and T. R. Cummins, Painful Research: Identification of a Small-Molecule Inhibitor that Selectively Targets Na_{V}1.8 Sodium Channels. Mol. Interv., 2007. 7(4): p. 192-5); England, S., Voltage-gated sodium channels: the search for subtype-selective analgesics. Expert Opin. Investig. Drugs 17 (12), p. 1849-64 (2008); Krafte, D. S. and Bannon, A. W., Sodium channels and nociception: recent concepts and therapeutic opportunities. Curr. Opin. Pharmacol. 8 (1), p. 50-56 (2008)). Na_{V}s mediate the rapid upstroke of the action potential of many excitable cell types (e.g. neurons, skeletal myocytes, cardiac myocytes), and thus are involved in the initiation of signaling in those cells (Hille, Bertil, Ion Channels of Excitable Membranes, Third ed. (Sinauer Associates, Inc., Sunderland, MA, 2001)). Because of the role Na_{V}s play in the initiation and propagation of neuronal signals, antagonists that reduce Na_{V} currents can prevent or reduce neural signaling and Na_{V} channels have been considered likely targets to reduce pain in conditions where hyper-excitability is observed (Chahine, M., Chatelier, A., Babich, O., and Krupp, J. J., Voltage-gated sodium channels in neurological disorders. CNS Neurol. Disord. Drug Targets 7 (2), p. 144-58 (2008)). Several clinically useful analgesics have been identified as inhibitors of Na_{V} channels. The local anesthetic drugs such as lidocaine block pain by inhibiting Na_{V} channels, and other compounds, such as carbamazepine, lamotrigine, and tricyclic antidepressants that have proven effective at reducing pain have also been suggested to act by sodium channel inhibition (Soderpalm, B., Anticonvulsants: aspects of their mechanisms of action. Eur. J Pain 6 Suppl. A, p. 3-9 (2002); Wang, G. K., Mitchell, J., and Wang, S. Y., Block of persistent late Na⁺ currents by antidepressant sertraline and paroxetine. J Membr. Biol. 222 (2), p. 79-90 (2008)).

The Na_{V}s form a subfamily of the voltage-gated ion channel super-family and comprises 9 isoforms, designated Na_{V}1.1-Na_{V}1.9. The tissue localizations of the nine isoforms vary. Na_{V}1.4 is the primary sodium channel of skeletal muscle, and Na_{V}1.5 is primary sodium channel of cardiac myocytes. Na_{V}s 1.7, 1.8 and 1.9 are primarily localized to the peripheral nervous system, while Na_{V}s 1.1, 1.2, 1.3, and 1.6 are neuronal channels found in both the central and peripheral nervous systems. The functional behaviors of the nine isoforms are similar but distinct in the specifics of their voltage-dependent and kinetic behavior (Catterall, W. A., Goldin, A. L., and Waxman, S. G., International Union of Pharmacology. XLVII. Nomenclature and structure-function relationships of voltage-gated sodium channels. Pharmacol. Rev. 57 (4), p. 397 (2005)).

Upon their discovery, Na_{V}1.8 channels were identified as likely targets for analgesia (Akopian, A. N., L. Sivilotti, and J. N. Wood, A tetrodotoxin-resistant voltage-gated sodium channel expressed by sensory neurons. Nature, 1996. 379(6562): p. 257-62). Since then, Na_{V}1.8 has been shown to be a carrier of the sodium current that maintains action potential firing in small dorsal root ganglia (DRG) neurons (Blair, N. T. and B. P. Bean, Roles of tetrodotoxin (TTX)-sensitive Na⁺ current, TTX-resistant Na⁺ current, and Ca² current in the action potentials of nociceptive sensory neurons. J Neurosci., 2002. 22(23): p. 10277-90). Na_{V}1.8 is involved in spontaneous firing in damaged neurons, like those that drive neuropathic pain (Roza, C., et al., The tetrodotoxin-resistant Na⁺ channel Na_{V}1.8 is essential for the expression of spontaneous activity in damaged sensory axons of mice. J. Physiol., 2003. 550(Pt 3): p. 921-6; Jarvis, M. F., et al., A-803467, a potent and selective Na_{V}1.8 sodium channel blocker, attenuates neuropathic and inflammatory pain in the rat. Proc. Natl. Acad. Sci. USA, 2007. 104(20): p. 8520-5; Joshi, S. K., et al., Involvement of the TTX-resistant sodium channel Na_{V}1.8 in inflammatory and neuropathic, but not post-operative, pain states. Pain, 2006. 123(1-2): pp. 75-82; Lai, J., et al., Inhibition of neuropathic pain by decreased expression of the tetrodotoxin-resistant sodium channel, Na_{V}1.8. Pain, 2002. 95(1-2): p. 143-52; Dong, X. W., et al., Small interfering RNA-mediated selective knockdown of Na_{V}1.8 tetrodotoxin-resistant sodium channel reverses mechanical allodynia in neuropathic rats. Neuroscience, 2007. 146(2): p. 812-21; Huang, H. L., et al., Proteomic profiling of neuromas reveals alterations in protein composition and local protein synthesis in hyper-excitable nerves. Mol. Pain, 2008. 4: p. 33; Black, J. A., et al., Multiple sodium channel isoforms and mitogen-activated protein kinases are present in painful human neuromas. Ann. Neurol., 2008. 64(6): p. 644-53; Coward, K., et al., Immunolocalization of SNS/PN3 and NaN/SNS2 sodium channels in human pain states. Pain, 2000. 85(1-2): p. 41-50; Yiangou, Y., et al., SNS/PN3 and SNS2/NaN sodium channel-like immunoreactivity in human adult and neonate injured sensory nerves. FEBS Lett., 2000. 467(2-3): p. 249-52; Ruangsri, S., et al., Relationship of axonal voltage-gated sodium channel 1.8 (Na_{V}1.8) mRNA accumulation to sciatic nerve injury-induced painful neuropathy in rats. J. Biol. Chem. 286(46): p. 39836-47). The small DRG neurons where Na_{V}1.8 is expressed include the nociceptors involved in pain signaling. Na_{V}1.8 mediates large amplitude action potentials in small neurons of the dorsal root ganglia (Blair, N. T. and B. P. Bean, Roles of tetrodotoxin (TTX)-sensitive Na⁺ current, TTX-resistant Na⁺ current, and Ca⁺ current in the action potentials of nociceptive sensory neurons. J. Neurosci., 2002. 22(23): p. 10277-90). Na_{V}1.8 is necessary for rapid repetitive action potentials in nociceptors, and for spontaneous activity of damaged neurons. (Choi, J. S. and S. G. Waxman, Physiological interactions between Na_{V}1.7 and Na_{V}1.8 sodium channels: a computer simulation study. J. Neurophysiol. 106(6): p. 3173-84; Renganathan, M., T. R. Cummins, and S. G. Waxman, Contribution of Na(_{V})_{1.8} sodium channels to action potential electrogenesis in DRG neurons. J. Neurophysiol., 2001. 86(2): p. 629-40; Roza, C., et al., The tetrodotoxin-resistant Na⁺ channel Na_{V}1.8 is essential for the expression of spontaneous activity in damaged sensory axons of mice. J. Physiol., 2003. 550(Pt 3): p. 921-6). In depolarized or damaged DRG neurons, Na_{V}1.8 appears to be a driver of hyper-excitablility (Rush, A. M., et al., A single sodium channel mutation produces hyper- or hypoexcitability in different types of neurons. Proc. Natl. Acad. Sci. USA, 2006. 103(21): p. 8245-50). In some animal pain models, Na_{V}1.8 mRNA expression levels have been shown to increase in the DRG (Sun, W., et al., Reduced conduction failure of the main axon of polymodal nociceptive C-fibers contributes to painful diabetic neuropathy in rats. Brain, 135(Pt 2): p. 359-75; Strickland, I. T., et al., Changes in the expression of Na_{V}1.7, Na_{V}1.8 and Na_{V}1.9 in a distinct population of dorsal root ganglia innervating the rat knee joint in a model of chronic inflammatory joint pain. Eur. J. Pain, 2008. 12(5): p. 564-72; Qiu, F., et al., Increased expression of tetrodotoxin-resistant sodium channels Na_{V}1.8 and Na_{V}1.9 within dorsal root ganglia in a rat model of bone cancer pain. Neurosci. Lett., 512(2): p. 61-6).

The inventors have discovered that some voltage-gated sodium channel inhibitors have limitations as therapeutic agents due to, for example, a poor therapeutic window (e.g., due to a lack of Na_{V} isoform selectivity, low potency, and/or other reasons). Accordingly, there remains a need to develop selective voltage-gated sodium channel inhibitors, such as selective Na_{V}1.8 inhibitors.

### SUMMARY

In one aspect, the invention relates to a compound described herein, or a pharmaceutically acceptable salt thereof.

In another aspect, the invention relates to a pharmaceutical composition comprising the compound, or a pharmaceutically acceptable salt thereof, and one or more pharmaceutically acceptable carriers or vehicles.

In still another aspect, the invention relates to a method of inhibiting a voltage gated sodium channel in a subject by administering the compound, pharmaceutically acceptable salt, or pharmaceutical composition to the subject.

In yet another aspect, the invention relates to a method of treating or lessening the severity in a subject of a variety of diseases, disorders, or conditions, including, but not limited to, chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, and cardiac arrhythmia, by administering the compound, pharmaceutically acceptable salt, or pharmaceutical composition to the subject.

### DETAILED DESCRIPTION

In one aspect, the invention relates to a compound of formula (I):

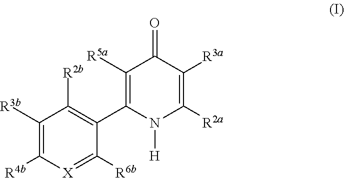

or a pharmaceutically acceptable salt thereof, wherein:
   X is CR^{5b} or N;
R^{2a} and R^{3a} are defined as follows:
   (i) R^{2a} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂; and
      R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂), —C(O)NHC(O)(C₁-C₆ alkyl), —NHS(O)₂CH₃, —S(C₁-C₆ alkyl), C₃-C₇ cycloalkyl, C₆-C₁₀ aryl, 4-10 membered heterocyclyl, 4-10 membered heteroaryl, wherein said heterocyclyl or heteroaryl is optionally substituted with 1-2 R^{a′}; or
   (ii) R^{3a} is —S(O)R′, —S(O)₂R′, —S(O)(NH)R′, —S(O)(N(C₁-C₆ alkyl))R′, or —S(O)₂NCH₃R′; and
      R^{2a} and R′, together with the atoms to which they are attached, join together to form a 4-7 membered heterocyclyl;
   R^{5a} is H, halo, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), —C(O)O(C₁-C₆ haloalkyl), or 4-10 membered heteroaryl, wherein said heteroaryl is optionally substituted with R^{a′};
   each R^{a′} is independently halo, —CN, —OH, oxo, C₁-C₆ alkyl, C₁-C₆ haloalkyl, C₁-C₆ alkoxy, —(C₁-C₆ alkylene)-(C₁-C₆ alkoxy), —C(O)NH₂, —C(O)OH, —S(O)₂(C₁-C₆ alkyl), C₃-C₇ cycloalkyl, 4-10 membered heterocyclyl, or -(4-10 membered heterocyclyl)-(C₁-C₆ alkyl);
R^{2b}, R^{3b}, and R^{4b} are defined as follows:
   (i) R^{2b} is H, halo, C₁-C₆ alkyl, C₁-C₆ alkoxy, —OH, —CH₂OH, —OCH₂CH₂OH, —OCH₂CH₂OCH₃, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
      R^{3b} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or —(C₁-C₆ haloalkyl)-(C₃-C₆ cycloalkyl), wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
      R^{4b} is H, halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, (C₁-C₆ haloalkyl)-(C₃-C₆ cycloalkyl)-, or

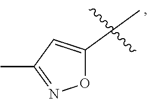 [SMILES: *c1cc(C)no1]

wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; or
(ii) R^{2b} and R^{3b}, together with the carbon atoms to which they are attached, form a ring of formula:

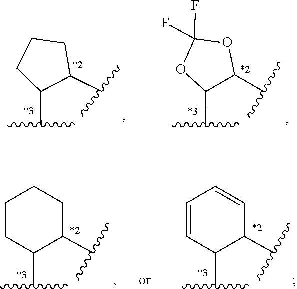 [SMILES: *C1CCCC1* ; *C1OC(F)(F)OC1* ; *C1CCCCC1* ; *C1C=CC=CC1*]

and
   R^{4b} is H, halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or —(C₁-C₆ haloalkyl)-(C₃-C₆ cycloalkyl), wherein said cycloalkyl is optionally substituted with one or more halogen; or
(iii) R^{2b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
   R^{3b} and R^{4b}, together with the carbon atoms to which they are attached, form a ring of formula:

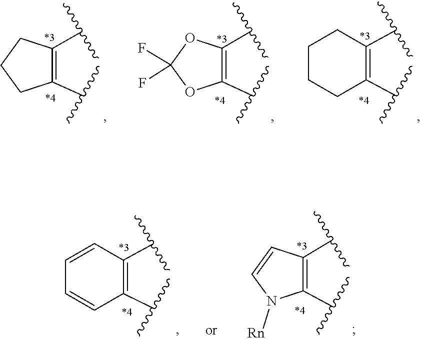

R^{5b} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
R^{6b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
Rₐ is H or C₁-C₆ alkyl,
provided that:
   (i) if X is N, then R^{3b} and R^{4b}, together with the carbon atoms to which they are attached, form a ring of formula:

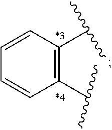 [SMILES: *c1ccccc1*]

and
(ii) if R^{2a} is H, then R^{3a} is halo, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
(iii) if R^{3a} is —C(O)O(C₁-C₂ alkyl), then R^{4b} is H, fluoro, chloro, C₁-C₆ alkyl, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(iv) no more than four of R^{2b}, R^{3b}, R^{4b}, R^{5b}, and R^{6b} are H; and
(v) no more than one of R^{2b}, R^{3b}, R^{4b}, R^{5b}, and R^{6b} is fluoro; and
(vi) no more than one of R^{2b}, R^{3b}, R^{4b}, R^{5b}, and R^{6b} is chloro; and
(vii) if any of R^{2b}, R^{3b}, R^{4b}, R^{5b}, and R^{6b} are halo, then no more than three of R^{2b}, R^{3b}, R^{4b}, R^{5b}, and R^{6b} are H; and
(viii) if R^{2b} or R^{6b} is —OCH₃ and R^{3a} is H, then no more than three of R^{2b}, R^{3b}, R^{4b}, R^{5b}, and R^{6b} are H; and
(ix) if R^{3b} is fluoro, then R^{6b} is H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(x) if R^{5b} is fluoro, then R² is H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(xi) if R^{3b} or R is —CH₃, then no more three of R^{2b}, R^{3b}, R^{4b}, R^{5b}, and R^{6b} are H; and
(xii) if R^{3b} or R is C₁ haloalkyl, then no more than three of R^{2b}, R^{3b}, R^{4b}, R^{5b}, and R^{6b} are H; and
(xiii) if R^{3b} or R^{5b} is —OCH₃, then no more than three of R^{2b}, R^{3b}, R^{4b}, R^{5b}, and R^{6b} are H; and
(xiv) if R^{4b} is C₁ haloalkyl, then no more than three of R^{2b}, R^{3b}, R^{5b} and R^{6b} are H; and
(xv) if R^{4b} is —OCH₃, then no more than three of R^{2b}, R^{3b}, R^{5b} and R^{6b} are H; and
(xvi) if R^{4b} is C₁ haloalkoxy, then no more than three of R^{2b}, R^{3b}, R^{5b} and R^{6b} are H; and
(xvii) if R^{4b} is chloro or bromo, then R^{2b} and R^{6b} are each independently H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(xviii) if R^{4b} and R^{3b} are each —OCH₃, then no more than two of R^{2b}, R^{5b}, and R^{6b} are H; and
(xix) if R^{4b} and R^{5b} are each —OCH₃, then no more than two of R^{2b}, R^{3b}, and R^{6b} are H.

In another aspect, the invention relates to a compound of formula (II):

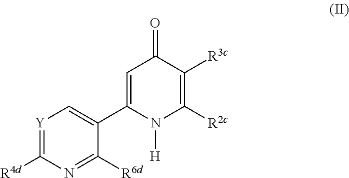

or a pharmaceutically acceptable salt thereof, wherein:
   Y is CR^{3d} or N;
   R^{2c} is C₁-C₆ alkyl;
   R^{3C} is H, halo, C₁-C₆ alkyl, —S(O)₂(C₁-C₆ alkyl), —S(O)(NH)(C₁-C₆ alkyl), or 4-10 membered heteroaryl, wherein said heteroaryl is optionally substituted with C₁-C₆ alkyl;
   R^{3d} is halo, C₁-C₆ alkyl, or C₁-C₆ haloalkyl;
   R^{4d} is H, C₁-C₆ alkyl, or C₁-C₆ haloalkyl; and
   R^{6d} is H, C₁-C₆ alkyl, or C₁-C₆ alkoxy.

In still another aspect, the invention relates to a compound of formula (III):

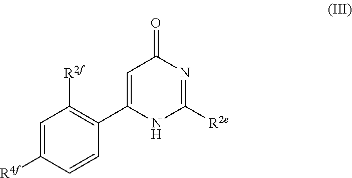

or a pharmaceutically acceptable salt thereof, wherein:
   R^{2b} is C₁-C₆ alkyl;
   R^{2f} is C₁-C₆ alkyl; and
   R^{4f} is C₁-C₆ alkyl.

In yet another aspect, the invention relates to a compound of formula (IV):

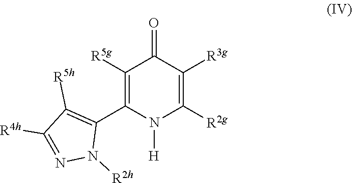

or a pharmaceutically acceptable salt thereof, wherein:
R^{2g} and R^{3g} are defined as follows:
   (i) R^{2g} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂; and
      R^{3g} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂), —C(O)NHC(O)(C₁-C₆ alkyl), —NHS(O)₂CH₃, —S(C₁-C₆ alkyl), C₃-C₇ cycloalkyl, C₆-C₁₀ aryl, 4-10 membered heterocyclyl, 4-10 membered heteroaryl, wherein said heterocyclyl or heteroaryl is optionally substituted with 1-2 R^{g′}; or
   (ii) R^{3g} is —S(O)R′, —S(O)₂R′, —S(O)(NH)R′, —S(O)(N(C₁-C₆ alkyl))R′, or —S(O)₂NCH₃R′; and
      R^{2g} and R′, together with the atoms to which they are attached, join together to form a 4-7 membered heterocyclyl;
   R^{5g} is H, halo, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), —C(O)O(C₁-C₆ haloalkyl), or 4-10 membered heteroaryl, wherein said heteroaryl is optionally substituted with R^{g′};
   each R^{g′} is independently halo, —CN, —OH, oxo, C₁-C₆ alkyl, C₁-C₆ haloalkyl, C₁-C₆ alkoxy, —(C₁-C₆ alkylene)-(C₁-C₆ alkoxy), —C(O)NH₂, —C(O)OH, —S(O)₂(C₁-C₆ alkyl), C₃-C₇ cycloalkyl, 4-10 membered heterocyclyl, or -(4-10 membered heterocyclyl)-(C₁-C₆ alkyl);
   R^{2h} is H, C₁-C₆ alkyl, C₁-C₆ alkoxy, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
R^{4h} and R^{5h} are defined as follows:
   (i) R^{4h} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or —(C₁-C₆ haloalkyl)-(C₃-C₆ cycloalkyl), wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
      R^{5h} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or —(C₁-C₆ haloalkyl)-(C₃-C₆ cycloalkyl), wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; or
   (ii) R^{4h} and R^{5h}, together with the C atoms to which they are attached, join together to form a C₅-C₆ cycloalkyl, wherein said cycloalkyl is optionally substituted with 1-2 C₁-C₆ alkyl.

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As used herein, the term “compounds of the invention” refers to the compounds of formulas (I), (II), (III), (IV) and all of the embodiments thereof (e.g., formulas (I-A-1), (I-A-2), (I-B-1), (I-B-2), (I-C-1), (I-C-2), (I-D-1), (I-D-2), (I-E-1), (I-E-2), (I-F), (II-A), etc.), as described herein, and to the compounds identified in Table A, Table B, Table C, and Table D.

As described herein, the compounds of the invention comprise multiple variable groups (e.g., X, R^{2a}, Y etc.). As one of ordinary skill in the art will recognize, combinations of groups envisioned by this invention are those combinations that result in the formation of stable or chemically feasible compounds. The term “stable,” in this context, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The chemical structures depicted herein are intended to be understood as they would be understood by one of ordinary skill in the art. For example, a substituent depicted as “CF₃” or “F₃C” in a chemical structure refers to a trifluoromethyl substituent, regardless of which depiction appears in the chemical structure.

As used herein, the term “halo” means F, Cl, Br or I.

As used herein, the term “alkyl” refers to a straight or branched hydrocarbon chain radical group consisting solely of carbon and hydrogen atoms, containing no unsaturation, and having the specified number of carbon atoms, which is attached to the rest of the molecule by a single bond. For example, a “C₁-C₆ alkyl” group is an alkyl group having between one and six carbon atoms.

As used herein, the term “cycloalkyl” refers to a stable, non-aromatic, mono- or bicyclic (fused, bridged, or spiro) saturated hydrocarbon radical consisting solely of carbon and hydrogen atoms, having the specified number of carbon ring atoms, and which is attached to the rest of the molecule by a single bond. For example, a “C₃-C₆ cycloalkyl” group is a cycloalkyl group having between three and eight carbon atoms.

As used herein, the term “alkoxy” refers to a radical of the formula —ORₐ where Rₐ is an alkyl group having the specified number of carbon atoms. For example, a “C₁-C₆ alkoxy” group is a radical of the formula —ORₐ where Rₐ is an alkyl group having the between one and six carbon atoms.

As used herein, the term “haloalkyl” refers to an alkyl group having the specified number of carbon atoms, wherein one or more of the hydrogen atoms of the alkyl group are replaced by halo groups. For example, a “C₁-C₆ haloalkyl” group is an alkyl group having between one and six carbon atoms, wherein one or more of the hydrogen atoms of the alkyl group are replaced by halo groups.

As used herein, the term “haloalkoxy” refers to an alkoxy group having the specified number of carbon atoms, wherein one or more of the hydrogen atoms of the of the alkyl group are replaced by halo groups.

As used herein, the term “alkylene” refers to a divalent, straight or branched hydrocarbon chain radical group consisting solely of carbon and hydrogen atoms, containing no unsaturation, and having the specified number of carbon atoms, which is attached to the rest of the molecule by two single bonds. For example, a “C₁-C₆ alkylene” group is an alkylene group having between one and six carbon atoms.

As used herein, the term “haloalkylene” refers to an alkylene group having the specified number of carbon atoms, wherein one or more of the hydrogen atoms of the alkylene group are replaced by halo groups. For example, a “C₁-C₆ haloalkylene” group is an alkylene group having between one and six carbon atoms, wherein one or more of the hydrogen atoms of the alkylene group are replaced by halo groups.

As used herein, the term “heterocyclyl” refers to a stable, non-aromatic, mono-, bi-, or tricyclic (fused, bridged, or spiro) radical in which one or more ring atoms is a heteroatom (e.g., a heteroatom independently selected from N, O, P, and S), which has the specified number of ring atoms, and which is attached to the rest of the molecule by a single bond. Heterocyclic rings can be saturated, or can contain one or more double or triple bonds. In some embodiments, the “heterocyclyl” group has the indicated number of ring members, in which one or more ring members is a heteroatom independently selected from oxygen, sulfur, nitrogen, and phosphorus, and each ring in the ring system contains 3 to 7 ring members. For example, a 6-membered heterocyclyl includes a total of 6 ring members, at least one of which is a heteroatom (e.g., a heteroatom independently selected from N, O, P, and S).

As used herein, the term “heteroaryl” refers to a stable mono-, bi-, or tricyclic ring radical having the specified number of ring atoms, wherein at least one ring in the system is aromatic, at least one aromatic ring in the system contains one or more heteroatoms (e.g., one or more heteroatoms independently selected from N, O, P, and S). In some embodiments, each ring in the system contains 3 to 7 ring members. For example, a 6-membered heteroaryl includes a total of 6 ring members, at least one of which is a heteroatom selected from N, S, O, and P. The term “heteroaryl” may be used interchangeably with the term “heteroaryl ring” or the term “heteroaromatic”.

As used herein, labels such as “*2” and “*3”, such as those in the following structures, designate the carbon atoms to which the corresponding R groups (in this case, the R^{2b} and R^{3b} groups, respectively) are attached:

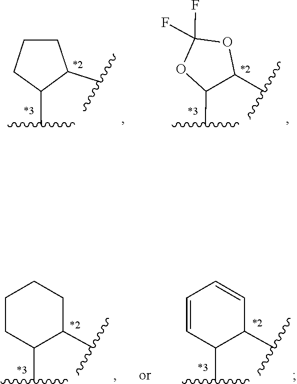 [SMILES: *C1CCCC1* ; *C1OC(F)(F)OC1* ; *C1CCCCC1* ; *C1C=CC=CC1*]

Similarly, “*3” and “*4” in the following structures designate the carbon atoms to which the R^{3b} and R^{4b} groups, respectively, are attached:

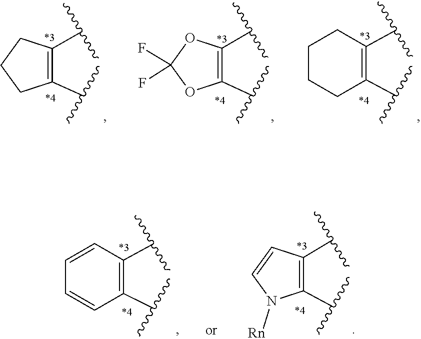

Unless otherwise specified, the compounds of the invention, whether identified by chemical name or chemical structure, include all stereoisomers (e.g., enantiomers and diastereomers), double bond isomers (e.g., (Z) and (E)), conformational isomers, and tautomers of the compounds identified by the chemical names and chemical structures provided herein. In addition, single stereoisomers, double bond isomers, conformational isomers, and tautomers as well as mixtures of stereoisomers, double bond isomers, conformational isomers, and tautomers are within the scope of the invention.

As used herein, in any chemical structure or formula, a non-bold, straight bond attached to a stereocenter of a compound, such as in

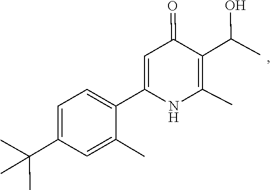 [SMILES: Cc1cc(C(C)(C)C)ccc1-c1cc(=O)c(C(C)O)c(C)[nH]1]

denotes that the configuration of the stereocenter is unspecified. The compound may have any configuration, or a mixture of configurations, at the stereocenter.

As used herein, the prefix “rac-,” when used in connection with a chiral compound, refers to a racemic mixture of the compound.

As used herein, the prefix “rel-,” when used in connection with a chiral compound, refers to a single enantiomer of unknown absolute configuration. In a compound bearing the “rel-” prefix, the (R)- and (S)- designators in the chemical name reflect the relative stereochemistry of the compound, but do not necessarily reflect the absolute stereochemistry of the compound. Where the relative stereochemistry of a given stereocenter is unknown, no stereochemical designator is provided.

As used herein, the term “compound,” when referring to the compounds of the invention, refers to a collection of molecules having identical chemical structures, except that there may be isotopic variation among the constituent atoms of the molecules. The term “compound” includes such a collection of molecules without regard to the purity of a given sample containing the collection of molecules. Thus, the term “compound” includes such a collection of molecules in pure form, in a mixture (e.g., solution, suspension, colloid, or pharmaceutical composition, or dosage form) with one or more other substances, or in the form of a hydrate, solvate, or co-crystal.

In the specification and claims, unless otherwise specified, any atom not specifically designated as a particular isotope in any compound of the invention is meant to represent any stable isotope of the specified element. In the Examples, where an atom is not specifically designated as a particular isotope in any compound of the invention, no effort was made to enrich that atom in a particular isotope, and therefore a person of ordinary skill in the art would understand that such atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

As used herein in the specification and claims, “H” refers to hydrogen and includes any stable isotope of hydrogen, namely ¹H and D. In the Examples, where an atom is designated as “H,” no effort was made to enrich that atom in a particular isotope of hydrogen, and therefore a person of ordinary skill in the art would understand that such hydrogen atom likely was present at approximately the natural abundance isotopic composition of hydrogen.

As used herein, “¹H” refers to protium. Where an atom in a compound of the invention, or a pharmaceutically acceptable salt thereof, is designated as protium, protium is present at the specified position with at least the natural abundance concentration of protium.

As used herein, “D,” “d,” and “²H” refer to deuterium.

In some embodiments, the compounds of the invention, and pharmaceutically acceptable salts thereof, include each constituent atom at approximately the natural abundance isotopic composition of the specified element.

In some embodiments, the compounds of the invention, and pharmaceutically acceptable salts thereof, include one or more atoms having an atomic mass or mass number which differs from the atomic mass or mass number of the most abundant isotope of the specified element (“isotope-labeled” compounds and salts). Examples of stable isotopes which are commercially available and suitable for the invention include without limitation isotopes of hydrogen, carbon, nitrogen, oxygen, and phosphorus, for example ²H, ¹³C, ⁵N, ¹⁸O, ¹⁷O, and ³¹P, respectively.

The isotope-labeled compounds and salts can be used in a number of beneficial ways, including as medicaments. In some embodiments, the isotope-labeled compounds and salts are deuterium (²H)-labeled. Deuterium (²H)-labeled compounds and salts are therapeutically useful with potential therapeutic advantages over the non-²H-labeled compounds. In general, deuterium (²H)-labeled compounds and salts can have higher metabolic stability as compared to those that are not isotope-labeled owing to the kinetic isotope effect described below. Higher metabolic stability translates directly into an increased in vivo half-life or lower dosages, which under most circumstances would represent a preferred embodiment of the present invention. The isotope-labeled compounds and salts can usually be prepared by carrying out the procedures disclosed in the synthesis schemes, the Examples and the related description, replacing a non-isotope-labeled reactant by a readily available isotope-labeled reactant.

The deuterium (²H)-labeled compounds and salts can manipulate the rate of oxidative metabolism of the compound by way of the primary kinetic isotope effect. The primary kinetic isotope effect is a change of the rate for a chemical reaction that results from exchange of isotopic nuclei, which in turn is caused by the change in ground state energies of the covalent bonds involved in the reaction. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For example, if deuterium is bonded to a carbon atom at a non-exchangeable position, rate differences of k_{H}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of an isotope (e.g., deuterium) incorporated at a given position of an isotope-labeled compound of the invention, or a pharmaceutically acceptable salt thereof, may be defined by the isotopic enrichment factor. The term “isotopic enrichment factor,” as used herein, means the ratio between the abundance of an isotope at a given position in an isotope-labeled compound (or salt) and the natural abundance of the isotope.

Where an atom in a compound of the invention, or a pharmaceutically acceptable salt thereof, is designated as deuterium, such compound (or salt) has an isotopic enrichment factor for such atom of at least 3000 (˜45% deuterium incorporation). In some embodiments, the isotopic enrichment factor is at least 3500 (˜52.5% deuterium incorporation), at least 4000 (˜60% deuterium incorporation), at least 4500 (˜67.5% deuterium incorporation), at least 5000 (˜75% deuterium incorporation), at least 5500 (˜82.5% deuterium incorporation), at least 6000 (˜90% deuterium incorporation), at least 6333.3 (˜95% deuterium incorporation), at least 6466.7 (˜97% deuterium incorporation), at least 6600 (˜99% deuterium incorporation), or at least 6633.3 (˜99.5% deuterium incorporation).

In some embodiments, the invention relates to a compound of formula (I), or a pharmaceutically acceptable salt thereof, wherein:
R^{2a} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
R^{5a} is H, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl).

In some embodiments, the invention relates to a compound of formula (T), or a pharmaceutically acceptable salt thereof, wherein X is N. In some embodiments, the invention relates to a compound of formula (I), or a pharmaceutically acceptable salt thereof, wherein X is CR^{5b}. In some embodiments, the invention relates to a compound of formula (I), or a pharmaceutically acceptable salt thereof, wherein X is CR^{5b} and R^{5b} is H, halo, C₁-C₆ alkyl, C₁-C₆ haloalkyl, C₁-C₆ alkoxy, or —C(O)(C₁-C₆ alkyl). In other embodiments, R^{5b} is halo. In other embodiments, R^{Sb} is C₁-C₆ alkyl. In other embodiments, R^{5b} is C₁-C₆ haloalkyl. In other embodiments, R^{5b} is C₁-C₆ alkoxy. In other embodiments, R^{5b} is —C(O)(C₁-C₆ alkyl). In some embodiments, the invention relates to a compound of formula (I), or a pharmaceutically acceptable salt thereof, wherein X is CR^{5b} and R^{Sb} is H, F, Cl, —CH₃, —C(CH₃)₃, —CF₃, —OCH₃, or —C(O)CH₃. In other embodiments, R^{5b} is H. In other embodiments, R^{5b} is F. In other embodiments, R^{5b} is Cl. In other embodiments, R^{5b} is —CH₃. In other embodiments, R^{5b} is —C(CH₃)₃. In other embodiments, R^{5b} is —CF₃. In other embodiments, R^{Sb} is —OCH₃. In other embodiments, R^{5b} is —C(O)CH₃.

In some embodiments, the invention relates to a compound of formula (I), or a pharmaceutically acceptable salt thereof, wherein R^{2a} is H, C₁-C₆ alkyl, C₁-C₆ haloalkyl, C₁-C₆ alkoxy, or —N(C₁-C₆ alkyl)₂. In other embodiments, R^{2a} is C₁-C₆ alkyl. In other embodiments, R^{2a} is C₁-C₆ haloalkyl. In other embodiments, R^{2a} is C₁-C₆ alkoxy. In other embodiments, R^{2a} is —N(C₁-C₆ alkyl)₂. In some embodiments, the invention relates to a compound of formula (I), or a pharmaceutically acceptable salt thereof, wherein R^{2a} is H, —CH₃, —CF₃, —OCH₃, or —N(CH₃)₂. In other embodiments, R^{2a} is H. In other embodiments, R^{2a} is —CH₃. In other embodiments, R^{2a} is —CF₃. In other embodiments, R^{2a} is —OCH₃. In other embodiments, R^{2a} is —N(CH₃)₂.

In some embodiments, the invention relates to a compound of formula (I), or a pharmaceutically acceptable salt thereof, wherein R^{3a} is H, halo, —CN, C₁-C₆ alkyl, C₁-C₆ alkoxy, —NH₂, —N(C₁-C₆ alkyl)₂, —(C₁-C₆ alkylene)-OH, —(C₁-C₆ alkylene)-NH₂, —C(O)(C₁-C₆ alkyl), —C(O)NH₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂), —C(O)NHC(O)CH₃, —NHS(O)₂CH₃, —S(C₁-C₆ alkyl), C₃-C₇ cycloalkyl, C₆-C₁ₐ aryl, 4-10 membered heterocyclyl, 4-10 membered heteroaryl, wherein said heterocyclyl or heteroaryl is optionally substituted with 1-2 R^{a′}; and each R^{a′} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₃, cyclopropyl, azetidinyl, or 1-methylazetidinyl. In some embodiments, the invention relates to a compound of formula (I), or a pharmaceutically acceptable salt thereof, wherein R^{3a} is H, halo, —CN, C₁-C₆ alkyl, C₁-C₆ alkoxy, —NH₂, —N(C₁-C₆ alkyl)₂, —(C₁-C₆ alkylene)-OH, —(C₁-C₆ alkylene)-NH₂, —C(O)(C₁-C₆ alkyl), —C(O)NH₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂). In other embodiments, R^{3a} is halo. In other embodiments, R^{3a} is C₁-C₆ alkyl. In other embodiments, R^{3a} is C₁-C₆ alkoxy. In other embodiments, R^{3a} is —N(C₁-C₆ alkyl)₂. In other embodiments, R^{3a} is —(C₁-C₆ alkylene)-OH. In other embodiments, R^{3a} is —(C₁-C₆ alkylene)-NH₂. In other embodiments, R^{3a} is —C(O)(C₁-C₆ alkyl). In other embodiments, R^{3a} is —C(O)NH₂. In other embodiments, R^{3a} is —C(O)O(C₁-C₆ alkyl). In other embodiments, R^{3a} is —S(O)₂—(C₁-C₆ alkyl). In other embodiments, R^{3a} is —S(O)(NH)(C₁-C₆ alkyl). In other embodiments, R^{3a} is —CH₂C(O)NH(C₁-C₆ alkyl). In other embodiments, R^{3a} is —NHC(O)(C₁-C₆ alkyl). In other embodiments, R^{3a} is —C(O)NHC(O)CH₃. In other embodiments, R^{3a} is —NHS(O)₂CH₃. In other embodiments, R^{3a} is —S(C₁-C₆ alkyl). In other embodiments, R^{3a} is C₃-C₇ cycloalkyl. In other embodiments, R^{3a} is C₆-C₁₀ aryl. In other embodiments, R^{3a} is 4-10 membered heterocyclyl, wherein said heterocyclyl is optionally substituted with 1-2 R^{a′}; and each R^{a′} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₃, cyclopropyl, azetidinyl, or 1-methylazetidinyl. In other embodiments, R^{3a} is 4-10 membered heteroaryl, wherein said heteroaryl is optionally substituted with 1-2 R^{a′}; and each R^{a′} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₃, cyclopropyl, azetidinyl, or 1-methylazetidinyl. In some embodiments, the invention relates to a compound of formula (I), or a pharmaceutically acceptable salt thereof, wherein R^{3a} is H, Cl, Br, —CN, —CH₃, —CH(CH₃)₂, —OCH₃, —NH₂, —N(CH₃)₂, —CH₂OH, —CH(CH₃)(OH), —C(CH₃)₂(OH), —CH₂NH₂, —C(O)CH₃, —C(O)NH₂, —C(O)OCH₂CH₃, —S(O)₂—(CH₃), —S(O)₂CH(CH₃)₂, —S(O)(NH)(CH₃), —S(O)(NH)CH(CH₃)₂, —CH₂C(O)NH₂, —CH₂C(O)NH(CH₃), —NHC(O)CH₃, —CH₂NHC(O)CH(CH₃)(NH₂), —CH₂N(CH₃)C(O)CH(CH₃)(N(CH₃)₂), —CH₂CH₂NHC(O)CH(CH₂)(NH₂), —NHC(O)CH(CH₃)(NH₂), —C(O)NHC(O)CH₃, —NHS(H)₂CH₃, —SCH₃, cyclopropyl, phenyl

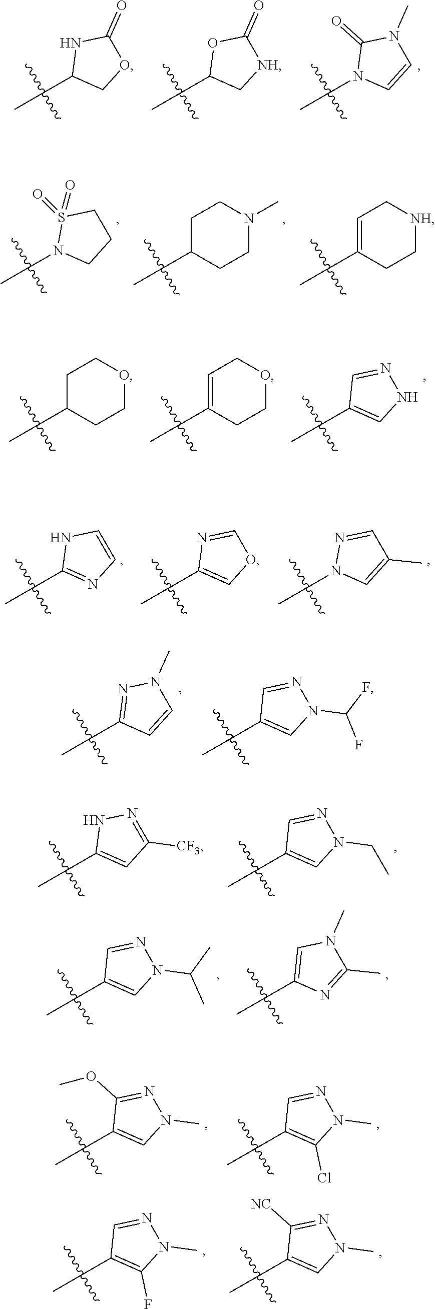

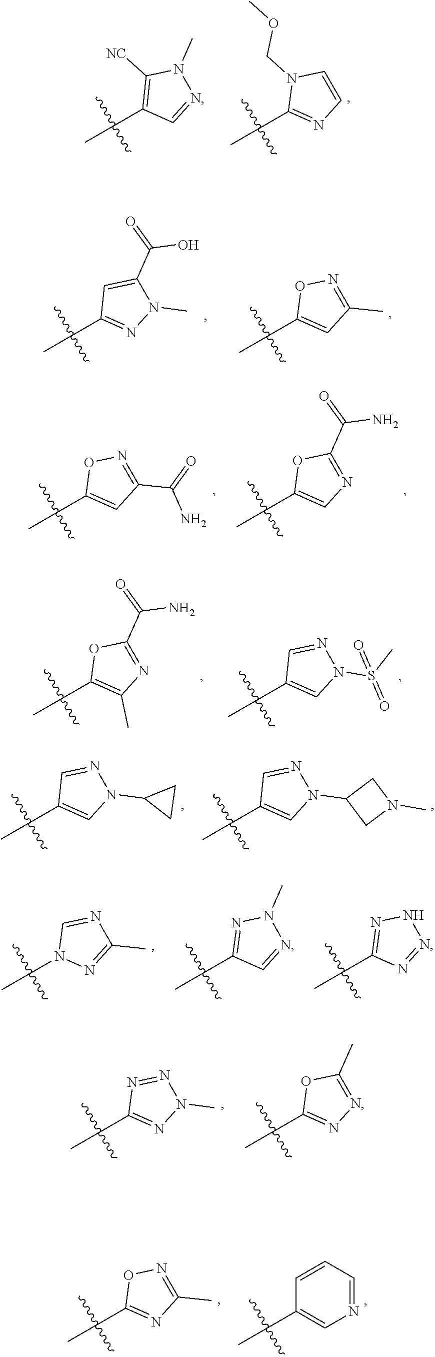

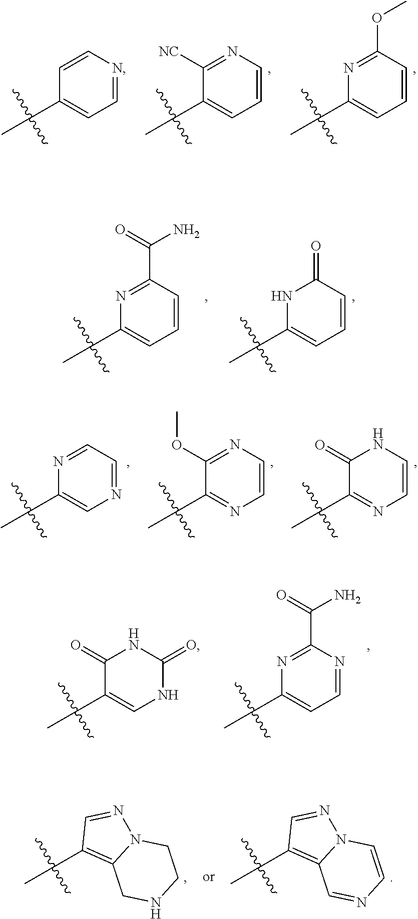

In some embodiments, the invention relates to a compound of formula (I), or a pharmaceutically acceptable salt thereof, wherein R^{3a} is H, Cl, —CN, —CH₃, —CH(CH₃)₂, —OCH₃, —NH₂, —N(CH₃)₂, —CH₂OH, —CH(CH₃)(OH), —C(CH₃)₂(OH), —CH₂NH₂, —C(O)CH₃, —C(O)NH₂, —C(O)OCH₂CH₃, —S(O)₂—(CH₃), —S(O)(NH)(CH₃), —CH₂C(O)NH₂, —CH₂C(O)NH(CH₃), —NHC(O)CH₃, —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂). In other embodiments, R^{3a} is H. In other embodiments, R^{3a} is Cl. In other embodiments, R^{3a} is —CN. In other embodiments, R^{3a} is —CH₃. In other embodiments, R^{3a} is —CH(CH₃)₂. In other embodiments, R^{3a} is —OCH₃. In other embodiments, R^{3a} is —NH₂. In other embodiments, R^{3a} is —N(CH₃)₂. In other embodiments, R^{3a} is —CH₂OH. In other embodiments, R^{3a} is —CH(CH₃)(OH). In other embodiments, R^{3a} is —C(CH₃)₂(OH). In other embodiments, R^{3a} is —CH₂NH₂. In other embodiments, R^{3a} is —C(O)CH₃. In other embodiments, R^{3a} is —C(O)NH₂. In other embodiments, R^{3a} is —C(O)OCH₂CH₃. In other embodiments, R^{3a} is —S(O)₂—(CH₃). In other embodiments, R^{3a} is —S(O)(NH)(CH₃). In other embodiments, R^{3a} is —CH₂C(O)NH₂. In other embodiments, R^{3a} is —CH₂C(O)NH(CH₃). In other embodiments, R^{3a} is —NHC(O)CH₃. In other embodiments, R^{3a} is —CH₂NHC(O)CH(CH₃)(NH₂). In other embodiments, R^{3a} is —CH₂N(CH₃)C(O)CH(CH₃)(N(CH₃)₂). In other embodiments, R^{3a} is —CH₂CH₂NHC(O)CH(CH₃)(NH₂). In other embodiments, R^{3a} is —NHC(O)CH(CH₃)(NH₂). In other embodiments, R^{3a} is Br. In other embodiments, R^{3a} is —S(O)₂CH(CH₃)₂. In other embodiments, R^{3a} is —S(O)(NH)CH(CH₃)₂. In other embodiments, R^{3a} is —C(O)NHC(O)CH₃. In other embodiments, R^{3a} is —NHS(O)₂CH₃. In other embodiments, R^{3a} is —SCH₃. In other embodiments, R^{3a} is cyclopropyl. In other embodiments, R^{3a} is phenyl. In other embodiments, R^{3a} is

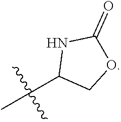 [SMILES: *C1COC(=O)N1]

In other embodiments, R^{3a} is

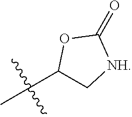 [SMILES: *C1CNC(=O)O1]

In other embodiments, R^{3a} is

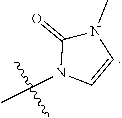 [SMILES: *n1ccn(C)c1=O]

In other embodiments, R^{3a} is

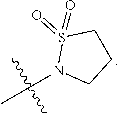 [SMILES: *N1CCCS1(=O)=O]

In other embodiments, R^{3a} is

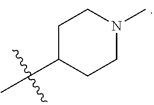 [SMILES: *C1CCN(C)CC1]

In other embodiments, R^{3a} is

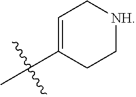 [SMILES: *C1=CCNCC1]

In other embodiments, R^{3a} is

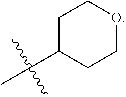 [SMILES: *C1CCOCC1]

In other embodiments, R^{3a} is

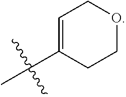 [SMILES: *C1=CCOCC1]

In other embodiments, R^{3a} is

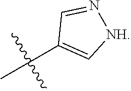 [SMILES: *c1cn[nH]c1]

In other embodiments, R^{3a} is

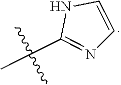 [SMILES: *c1ncc[nH]1]

In other embodiments, R^{3a} is

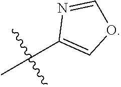 [SMILES: *c1cocn1]

In other embodiments, R^{3a} is

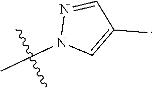 [SMILES: *n1cc(C)cn1]

In other embodiments, R^{3a} is

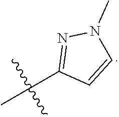 [SMILES: *c1ccn(C)n1]

In other embodiments, R^{3a} is

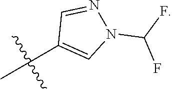 [SMILES: *c1cnn(C(F)F)c1]

In other embodiments, R^{3a} is

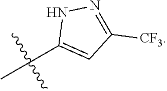 [SMILES: *c1cc(C(F)(F)F)n[nH]1]

In other embodiments. R^{3a} is

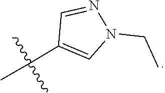 [SMILES: *c1cnn(CC)c1]

In other embodiments, R^{3a} is

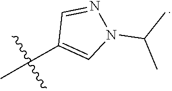 [SMILES: *c1cnn(C(C)C)c1]

In other embodiments, R^{3a} is

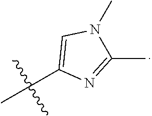 [SMILES: *c1cn(C)c(C)n1 ; "."]

In other embodiments, R^{3a} is

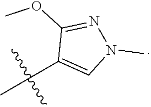

In other embodiments, R^{3a} is

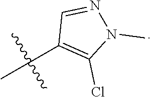 [SMILES: *c1cnn(C)c1Cl]

In other embodiments, R^{3a} is

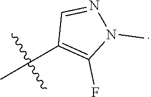 [SMILES: *c1cnn(C)c1F]

In other embodiments, R^{3a} is

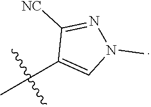 [SMILES: *c1cn(C)nc1C#N]

In other embodiments, R^{3a} is

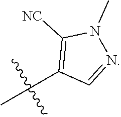 [SMILES: *c1cnn(C)c1C#N]

In other embodiments, R^{3a} is

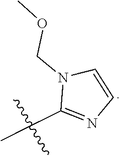 [SMILES: *c1nccn1COC]

In other embodiments, R^{3a} is

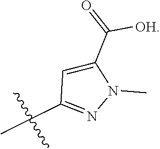 [SMILES: *c1cc(C(=O)O)n(C)n1]

In other embodiments, R^{3a} is

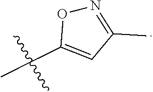 [SMILES: *c1cc(C)no1]

In other embodiments, R^{3a} is

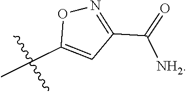 [SMILES: *c1cc(C(N)=O)no1]

In other embodiments, R^{3a} is

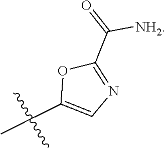 [SMILES: *c1cnc(C(N)=O)o1]

In other embodiments, R^{3a} is

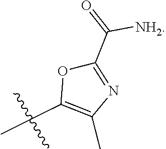 [SMILES: *c1oc(C(N)=O)nc1C]

In other embodiments, R^{3a} is

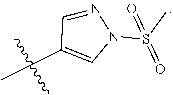 [SMILES: *c1cnn(S(C)(=O)=O)c1]

In other embodiments, R^{3a} is

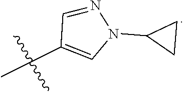 [SMILES: *c1cnn(C2CC2)c1]

In other embodiments, R^{3a} is

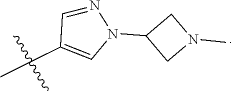 [SMILES: *c1cnn(C2CN(C)C2)c1]

In other embodiments, R^{3a} is

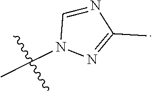 [SMILES: *n1cnc(C)n1]

In other embodiments, R^{3a} is

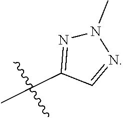 [SMILES: *c1cnn(C)n1]

In other embodiments. R^{3a} is

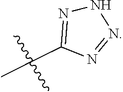 [SMILES: *c1nn[nH]n1]

In other embodiments, R^{3a} is

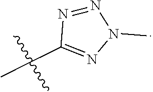 [SMILES: *c1nnn(C)n1]

In other embodiments, R^{3a} is

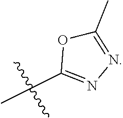 [SMILES: *c1nnc(C)o1]

In other embodiments, R^{3a} is

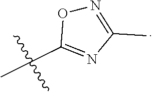 [SMILES: *c1nc(C)no1]

In other embodiments, R^{3a} is

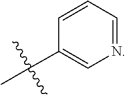 [SMILES: *c1cccnc1]

In other embodiments, R^{3a} is

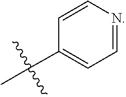 [SMILES: *c1ccncc1]

In other embodiments, R^{3a} is

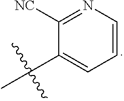 [SMILES: *c1cccnc1C#N]

In other embodiments, R^{3a} is

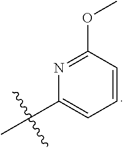 [SMILES: *c1cccc(OC)n1]

In other embodiments, R^{3a} is

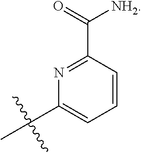 [SMILES: *c1cccc(C(N)=O)n1]

In other embodiments, R^{3a} is

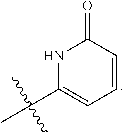 [SMILES: *c1cccc(=O)[nH]1]

In other embodiments, R^{3a} is

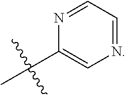 [SMILES: *c1cnccn1]

In other embodiments R^{3a} is

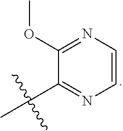 [SMILES: *c1nccnc1OC]

In other embodiments, R^{3a} is

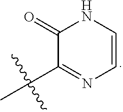 [SMILES: *c1ncc[nH]c1=O]

In other embodiments, R^{3a} is

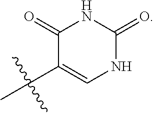 [SMILES: *c1c[nH]c(=O)[nH]c1=O]

In other embodiments, R^{3a} is

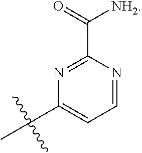 [SMILES: *c1ccnc(C(N)=O)n1]

In other embodiments, R^{3a} is

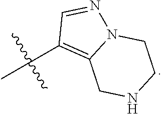 [SMILES: *c1cnn2c1CNCC2]

In other embodiments, R^{3a} is

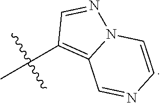 [SMILES: *c1cnn2ccncc12]

In some embodiments, the invention relates to a compound of formula (I), or a pharmaceutically acceptable salt thereof, wherein R^{3a} is —S(O)R′, —S(O)₂R′, —S(O)(NH)R′, —S(O)(NCH₃)R′, or —S(O)₂NCH₃R′; and R^{2a} and R′, together with the atoms to which they are attached, join together to form a 6-membered heterocyclyl. In other embodiments, R^{3a} is —S(O)R′; and R^{2a} and R′, together with the atoms to which they are attached, join together to form a 6-membered heterocyclyl. In other embodiments, R^{3a}—S(O)₂R′; and R^{2a} and R′, together with the atoms to which they are attached, join together to form a 6-membered heterocyclyl. In other embodiments, R^{3a}—S(O)(NH)R′; and R^{2a} and R′, together with the atoms to which they are attached, join together to form a 6-membered heterocyclyl. In other embodiments, R^{3a}—S(O)(NCH₃)R′; and R^{2a} and R′, together with the atoms to which they are attached, join together to form a 6-membered heterocyclyl. In other embodiments, R^{3a}—S(O)₂NCH₃R′; and R^{2a} and R′, together with the atoms to which they are attached, join together to form a 6-membered heterocyclyl.

In some embodiments, the invention relates to a compound of formula (I), or a pharmaceutically acceptable salt thereof, wherein R^{5a} is H, halo, C₁-C₆ alkyl, C₁-C₆ alkoxy, —CH₂OH, —CH₂O(C₁-C₆ alkyl), —C(O)O(C₁-C₆ alkyl), or 4-10 membered heteroaryl, wherein said heteroaryl is optionally substituted with R^{a′}; and R^{a′} is —CH₃, —CHF₂, or —CH₂OCH₃. In some embodiments, the invention relates to a compound of formula (I), or a pharmaceutically acceptable salt thereof, wherein R^{5a} is H, C₁-C₆ alkyl, C₁-C₆ alkoxy, —CH₂OH, —CH₂O(C₁-C₆ alkyl), or —C(O)O(C₁-C₆ alkyl). In other embodiments, R^{5a} is C₁-C₆ alkyl. In other embodiments, R^{5a} is C₁-C₆ alkoxy. In other embodiments, R^{5a} is —CH₂O(C₁-C₆ alkyl). In other embodiments, R^{5a} is —C(O)O(C₁-C₆ alkyl). In other embodiments, R^{5a} is halo. In other embodiments, R^{5a} is 4-10 membered heteroaryl, wherein said heteroaryl is optionally substituted with R^{a′}; and R^{a′} is —CH₃, —CHF₂, or —CH₂OCH₃. In some embodiments, the invention relates to a compound of formula (I), or a pharmaceutically acceptable salt thereof, wherein R^{5a} is H, Br, —CH₃, —OCH₃, —OCH₂CH₃, —CH₂OH, —CH₂OCH₃, —C(O)OCH₂CH₃,

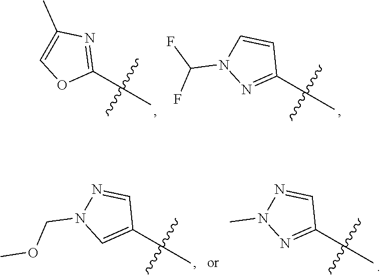

In some embodiments, the invention relates to a compound of formula (I), or a pharmaceutically acceptable salt thereof, wherein R^{5a} is H, —CH₃, —OCH₃, —OCH₂CH₃, —CH₂OH, —CH₂OCH₃, or —C(O)OCH₂CH₃. In other embodiments, R^{5a} is H. In other embodiments, R^{5a} is —CH₃. In other embodiments, R^{5a} is —OCH₃. In other embodiments, R^{5a} is —OCH₂CH₃. In other embodiments, R^{5a} is —CH₂OH. In other embodiments, R^{5a} is —CH₂OCH₃. In other embodiments, R^{5a} is —C(O)OCH₂CH₃. In other embodiments, R^{5a} is Br. In other embodiments, R^{5a} is

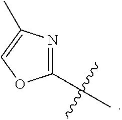 [SMILES: *c1nc(C)co1]

In other embodiments, R^{5a} is

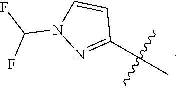 [SMILES: *c1ccn(C(F)F)n1]

In other embodiments, R^{5a} is

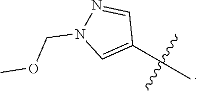 [SMILES: *c1cnn(COC)c1 ; "."]

In other embodiments, R^{5a} is

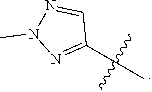 [SMILES: *c1cnn(C)n1 ; "."]

In some embodiments, the invention relates to a compound of formula (I), or a pharmaceutically acceptable salt thereof, wherein R^{2b} is H, halo, C₁-C₆ alkyl, C₁-C₆ alkoxy, —OH, —CH₂OH, —OCH₂CH₂OH, or —OCH₂CH₂OCH₃. In other embodiments, C₃-C₆ cycloalkyl. In other embodiments, R^{2b} is C₁-C₆ alkyl. In other embodiments, R^{2b} is C₁-C₆ alkoxy. In some embodiments, the invention relates to a compound of formula (I), or a pharmaceutically acceptable salt thereof, wherein R^{2b} is H, F, Cl, —CH₃, —CH(CH₃)₂, —C(CH₃)₃, —OCH₃, —OCH₂CH₃, —OH, —CH₂OH, —OCH₂CH₂OH, or —OCH₂CH₂OCH₃. In other embodiments, R^{2b} is H. In other embodiments, R^{2b} is F. In other embodiments, R^{2b} is Cl. In other embodiments, R^{2b} is —CH₃. In other embodiments, R^{2b} is —CH(CH₃)₂. In other embodiments, R^{2b} is —C(CH₃)₃. In other embodiments, R^{2b} is —OCH₃. In other embodiments, R^{2b}is —OCH₂CH₃. In other embodiments, R^{2′} is —OH. In other embodiments, R^{2b} is —CH₂OH. In other embodiments, R^{2b} is —OCH₂CH₂OH. In other embodiments, R^{2b} is —OCH₂CH₂OCH₃.

In some embodiments, the invention relates to a compound of formula (I), or a pharmaceutically acceptable salt thereof, wherein R^{3b} is H, halo, C₁-C₆ alkyl, C₁-C₆ haloalkyl, or C₁-C₆ alkoxy. In other embodiments, R^{3b} is halo. In other embodiments, R^{3b} is C₁-C₆ alkyl. In other embodiments, R^{3b} is C₁-C₆ haloalkyl. In other embodiments, R^{3b} is C₁-C₆ alkoxy. In some embodiments, the invention relates to a compound of formula (I), or a pharmaceutically acceptable salt thereof, wherein R^{3b} is H, F, Cl, —CH₃, —C(CH₃)₃, —CF₃, —OCH₃, —OCH₂CH₃, or —OC(CH₃)₃. In other embodiments, R^{3b} is H. In other embodiments, R^{3b} is F. In other embodiments, R^{3b} is Cl. In other embodiments, R^{3b} is —CH₃. In other embodiments, R^{3b} is —C(CH₃)₃. In other embodiments, R^{3b} is —CF₃. In other embodiments, R^{3b} is —OCH₃. In other embodiments, R^{3b} is —OCH₂CH₃. In other embodiments, R^{3b} is —OC(CH₃)₃.

In some embodiments, the invention relates to a compound of formula (I), or a pharmaceutically acceptable salt thereof, wherein R^{4b} is H, halo, C₁-C₆ alkyl, C₁-C₆ haloalkyl, C₁-C₆ alkoxy, C₁-C₆ haloalkoxy, C₃-C₆ cycloalkyl, (C₁-C₆ alkyl)-(C₃-C₆ cycloalkyl)-, (C₁-C₆ haloalkyl)-(C₃-C₆ cycloalkyl)-, or

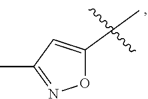 [SMILES: *c1cc(C)no1]

wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen. In other embodiments, R^{4b} is halo. In other embodiments, R^{4b} is C₁-C₆ alkyl. In other embodiments, R^{4b} is C₁-C₆ haloalkyl. In other embodiments, R^{4b} is C₁-C₆ alkoxy. In other embodiments, R^{4b} is C₁-C₆ haloalkoxy. In other embodiments, R^{4b} is C₃-C₆ cycloalkyl, wherein said cycloalkyl is optionally substituted with one or more halogen. In other embodiments, R^{4b} is (C₁-C₆ alkyl)-(C₃-C₆ cycloalkyl)-, wherein cycloalkyl in said (C₁-C₆ alkyl)-(C₃-C₆ cycloalkyl)- is optionally substituted with one or more halogen. In other embodiments, R^{4b} is (C₁-C₆ haloalkyl)-(C₃-C₆ cycloalkyl)-, wherein cycloalkyl in said (C₁-C₆ haloalkyl)-(C₃-C₆ cycloalkyl)- is optionally substituted with one or more halogen. In some embodiments, the invention relates to a compound of formula (I), or a pharmaceutically acceptable salt thereof, wherein R^{4b} is H, F, Cl, —CH₃, —CH(CH₃)₂, —C(CH₃)₃, —C(CH₃)₂(CH₂CH₃), —CF₃, —C(CH₃)₂(CF₃), —OCH₃, —OCH(CH₃)₂, —OC(CH₃)₃, —OCF₃, cyclopropyl, 1-trifluoromethylcyclopropyl, 3,3-difluorocyclobutyl, 3,3-difluoro-1-methylcyclobutyl, or

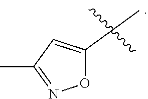 [SMILES: *c1cc(C)no1]

In other embodiments, Rib is H. In other embodiments, R^{4b} is F. In other embodiments, R^{4b} is Cl. In other embodiments, R^{4b} is —CH₃. In other embodiments, R^{4b} is —CH(CH₃)₂. In other embodiments, R^{4b} is —C(CH₃)₃. In other embodiments, R^{4b} is —C(CH₃)₂(CH₂CH₃). In other embodiments, Rib is —CF₃. In other embodiments, R^{4b} is —C(CH₃)₂(CF₃). In other embodiments, R^{4b} is —OCH₃. In other embodiments, R^{4b} is —OCH(CH₃)₂. In other embodiments, R^{4b} is —OC(CH₃)₃. In other embodiments, R^{4b} is —OCF₃. In other embodiments, R^{4b} is cyclopropyl. In other embodiments, R^{4b} is 1-trifluoromethylcyclopropyl. In other embodiments, R^{4b} is 3,3-difluorocyclobutyl. In other embodiments, R^{4b} is 3,3-difluoro-1-methylcyclobutyl. In other embodiments, R^{4b} is

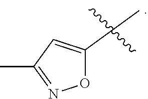 [SMILES: *c1cc(C)no1]

In some embodiments, the invention relates to a compound of formula (I), or a pharmaceutically acceptable salt thereof, wherein R^{4b} is —C(CD₃)₃. In some embodiments, the invention relates to a compound of formula (I), or a pharmaceutically acceptable salt thereof, wherein R^{4b} is —C(CD₃)(CH₃)(CF₃).

In some embodiments, the invention relates to a compound of formula (I), or a pharmaceutically acceptable salt thereof, wherein R^{2b} and R^{3b}, together with the carbon atoms to which they are attached, form a ring of formula

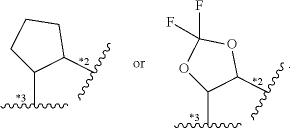 [SMILES: *C1CCCC1* ; *C1OC(F)(F)OC1*]

In some embodiments, the invention relates to a compound of formula (I), or a pharmaceutically acceptable salt thereof, wherein R^{2b} and R^{3b}, together with the carbon atoms to which they are attached, form a ring of formula

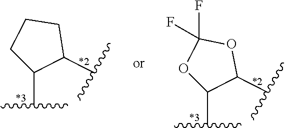 [SMILES: *C1CCCC1* ; *C1OC(F)(F)OC1*]

and R^{4b} is H. In other embodiments, R^{2b} and R^{3b}, together with the carbon atoms to which they are attached, form a ring of formula

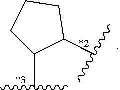 [SMILES: *C1CCCC1*]

In other embodiments, R^{2b} and R^{3b}, together with the carbon atoms to which they are attached, form a ring of formula

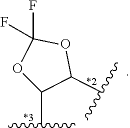 [SMILES: *C1OC(F)(F)OC1*]

In some embodiments, the invention relates to a compound of formula (I), or a pharmaceutically acceptable salt thereof, wherein R^{3b} and R^{4b}, together with the carbon atoms to which they are attached, form a ring of formula

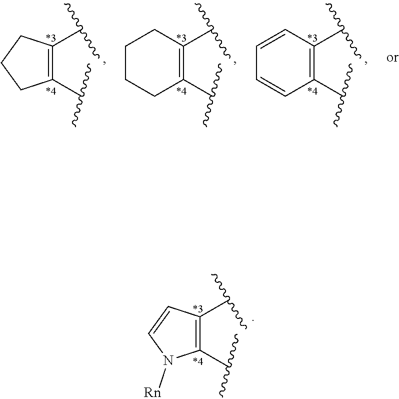

In some embodiments, the invention relates to a compound of formula (I), or a pharmaceutically acceptable salt thereof, wherein R^{3b} and R^{4b}, together with the carbon atoms to which they are attached, form a ring of formula

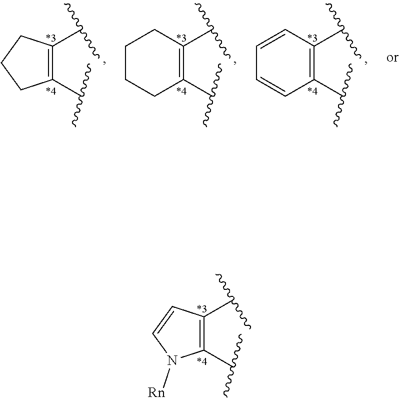

and R^{2b} is H. In other embodiments, R^{3b} and R^{4b}, together with the carbon atoms to which they are attached, form a ring of formula

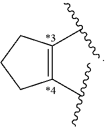 [SMILES: *C1=C(*)CCC1]

In other embodiments, R^{3b} and R^{4b}, together with the carbon atoms to which they are attached, form a ring of formula

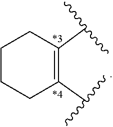 [SMILES: *C1=C(*)CCCC1]

In other embodiments, R^{3b} and R^{4b}, together with the carbon atoms to which they are attached, form a ring of formula

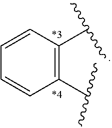 [SMILES: *c1ccccc1*]

In other embodiments, R^{3b} and R^{4b}, together with the carbon atoms to which they are attached, form a ring of formula

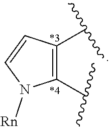

In some embodiments, the invention relates to a compound of formula (I), or a pharmaceutically acceptable salt thereof, wherein Rₐ is C₁-C₆ alkyl. In some embodiments, the invention relates to a compound of formula (I), or a pharmaceutically acceptable salt thereof, wherein Rₐ is —CH₃.

In some embodiments, the invention relates to a compound of formula (T), or a pharmaceutically acceptable salt thereof, wherein R^{6b} is H, halo, C₁-C₆ alkyl, or C₁-C₆ alkoxy. In other embodiments, R^{6b} is halo. In other embodiments, R^{6b} is C₁-C₆ alkyl. In other embodiments, R is C₁-C₆ alkoxy. In some embodiments, the invention relates to a compound of formula (I), or a pharmaceutically acceptable salt thereof, wherein R^{6b} is H, Cl, —CH₃, or —OCH₃. In other embodiments, R^{6b} is H. In other embodiments, R^{6b} is Cl. In other embodiments, R^{6b} is —CH₃. In other embodiments, R^{6b} is —OCH₃.

In some embodiments, the invention relates to a compound of formula (I-A-1):

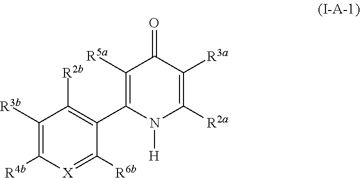

or a pharmaceutically acceptable salt thereof, wherein:
   X is CR^{5b} or N;
R^{2a} and R^{3a} are defined as follows:
   (i) R^{2a} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂; and
      R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂), —C(O)NHC(O)(C₁-C₆ alkyl), —NHS(O)₂CH₃, —S(C₁-C₆ alkyl), C₃-C₇ cycloalkyl, C₆-C₁₀ aryl, 4-10 membered heterocyclyl, 4-10 membered heteroaryl, and S, wherein said heterocyclyl or heteroaryl is optionally substituted with 1-2 R^{a′}; or
   (ii) R^{3a} is —S(O)R′, —S(O)₂R′, —S(O)(NH)R′, —S(O)(N(C₁-C₆ alkyl))R′, or —S(O)₂NCH₃R′; and
      R^{2a} and R′, together with the atoms to which they are attached, join together to form a 4-7 membered heterocyclyl;
   R^{5a} is H, halo, C₁-C₆ alkyl, C₁-C₆ haloalkyl, C₁-C₆ alkoxy, C₁-C₆ haloalkoxy; —CH₂OH, —CH₂O(C₁-C₆ alkyl), —CH₂O(C₁-C₆ haloalkyl), —C(O)O(C₁-C₆ alkyl), —C(O)O(C₁-C₆ haloalkyl), or 4-10 membered heteroaryl, wherein said heteroaryl is optionally substituted with R^{a′};
   each R^{a′} is independently halo, —CN, —OH, oxo, C₁-C₆ alkyl, C₁-C₆ haloalkyl, C₁-C₆ alkoxy, —(C₁-C₆ alkylene)-(C₁-C₆ alkoxy), —C(O)NH₂, —C(O)OH, —S(O)₂(C₁-C₆ alkyl), C₃-C₇ cycloalkyl, 4-10 membered heterocyclyl, or -(4-10 membered heterocyclyl)-(C₁-C₆ alkyl);
R^{2b}, R^{3b}, and R^{4b} are defined as follows:
   (i) R^{2b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
      R^{3b} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
      R^{4b} is H, halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; or
   (ii) R^{2b} and R^{3b}, together with the carbon atoms to which they are attached, form a ring of formula:

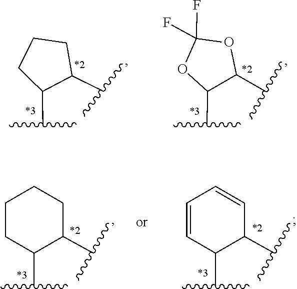 [SMILES: *C1CCCC1* ; *C1OC(F)(F)OC1* ; *C1CCCCC1* ; *C1C=CC=CC1*]

and
   R^{4b} is H, halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; or
(iii) R^{2b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
   R^{3b} and R^{4b}, together with the carbon atoms to which they are attached, form a ring of formula:

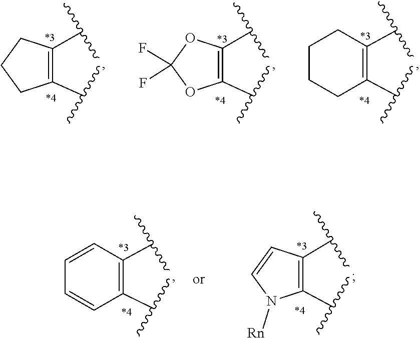

R^{5b} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
R^{6b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
Rₐ is H or C₁-C₆ alkyl,
provided that:
   (i) if X is N, then R^{3b} and R^{4b}, together with the carbon atoms to which they are attached, form a ring of formula:

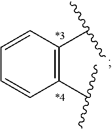 [SMILES: *c1ccccc1*]

(ii) if R^{2a} is H, then R^{3a} is halo, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
(iii) if R^{3a} is —C(O)O(C₁-C₂ alkyl), then R^{4b} is H, fluoro, chloro, C₁-C₆ alkyl, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(iv) no more than four of R^{2b}, R^{3b}, R^{4b}, R^{5b}, and R^{6b} are H; and
(v) no more than one of R^{2b}, R^{3b}, R^{4b}, R^{5b}, and R^{6b} is fluoro; and
(vi) no more than one of R^{2b}, R^{3b}, R^{4b}, R^{5b}, and R^{6b} is chloro; and
(vii) if any of R^{2b}, R^{3b}, R^{4b}, R^{5b}, and R^{6b} are halo, then no more than three of R^{2b}, R^{3b}, R^{4b}, R^{5b}, and R^{6b} are H; and
(viii) if R^{2b} or R^{6b} is —OCH₃ and R^{3a} is H, then no more than three of R^{2b}, R^{3b}, R^{4b}, R^{5b}, and R^{6b} are H; and
(ix) if R^{3b} is fluoro, then R^{6b} is H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(x) if R^{5b} is fluoro, then R^{2b} is H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(xi) if R^{3b} or R^{5b} is —CH₃, then no more three of R^{2b}, R^{3b}, R^{4b}, R^{5b}, and R^{6b} are H; and
(xii) if R^{3b} or R^{5b} is C₁ haloalkyl, then no more than three of R^{2b}, R^{3b}, R^{4b}, R^{5b}, and R^{6b} are H; and
(xiii) if R^{3b} or R^{5b} is —OCH₃, then no more than three of R^{2b}, R^{3b}, R^{4b}, R^{5b}, and R^{6b} are H; and
(xiv) if R^{4b} is C₁ haloalkyl, then no more than three of R^{2b}, R^{3b}, R^{5b} and R^{6b} are H, and
(xv) if R^{4b} is —OCH₃, then no more than three of R^{2b}, R^{3b}, R^{5b} and R^{6b} are H; and
(xvi) if R^{4b} is C₁ haloalkoxy, then no more than three of R^{2b}, R^{3b}, R^{5b} and R^{6b} are H; and
(xvii) if R^{4b} is chloro or bromo, then R^{2b} and R^{6b} are each independently H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(xviii) if R^{4b} and R^{3b} are each —OCH₃, then no more than two of R^{2b}, R^{5b}, and R^{6b} are H; and
(xix) if R^{4b} and Re are each —OCH₃, then no more than two of R^{2b}, R^{3b}, and R^{6b} are H.

In some embodiments, the invention relates to a compound of formula (I-A-1), or a pharmaceutically acceptable salt thereof, wherein:
R^{2a} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
R^{5a} is H, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl).

In some embodiments, the invention relates to a compound of formula (I-A-1), or a pharmaceutically acceptable salt thereof, wherein X is N. In some embodiments, the invention relates to a compound of formula (I-A-1), or a pharmaceutically acceptable salt thereof, wherein X is CR^{5b}. In some embodiments, the invention relates to a compound of formula (I-A-1), or a pharmaceutically acceptable salt thereof, wherein X is CR^{5b} and R^{5b} is H, halo, C₁-C₆ alkyl, C₁-C₆ haloalkyl, C₁-C₆ alkoxy, or —C(O)(C₁-C₆ alkyl). In other embodiments, R is halo. In other embodiments, R^{5b} is C₁-C₆ alkyl. In other embodiments, R is C₁-C₆ haloalkyl. In other embodiments, R^{5b} is C₁-C₆ alkoxy. In other embodiments, R^{5b} is —C(O)(C₁-C₆ alkyl). In some embodiments, the invention relates to a compound of formula (I-A-1), or a pharmaceutically acceptable salt thereof, wherein X is CR^{5b} and R^{5b} is H, F, Cl, —CH₃, —C(CH₃)₃, —CF₃, —OCH₃, or —C(O)CH₃. In other embodiments, R^{5b} is H. In other embodiments, R^{5b} is F. In other embodiments, R is Cl. In other embodiments, R^{5b} is —CH₃. In other embodiments, R^{5b} is —C(CH₃)₃. In other embodiments, R is —CF₃. In other embodiments, R^{5b} is —OCH₃. In other embodiments, R^{5b} is —C(O)CH₃.

In some embodiments, the invention relates to a compound of formula (I-A-1), or a pharmaceutically acceptable salt thereof, wherein R^{2a} is H, C₁-C₆ alkyl, C₁-C₆ haloalkyl, C₁-C₆ alkoxy, or —N(C₁-C₆ alkyl)₂. In other embodiments, R^{2a} is C₁-C₆ alkyl. In other embodiments, R^{2a} is C₁-C₆ haloalkyl. In other embodiments, R^{2a} is C₁-C₆ alkoxy. In other embodiments, R^{2a} is —N(C₁-C₆ alkyl)₂. In some embodiments, the invention relates to a compound of formula (I-A-1), or a pharmaceutically acceptable salt thereof, wherein R^{2a} is H, —CH₃, —CF₃, —OCH₃, or —N(CH₃)₂. In other embodiments, R^{2a} is H. In other embodiments, R^{2a} is —CH₃. In other embodiments, R^{2a} is —CF₃. In other embodiments, R^{2a} is —OCH₃. In other embodiments, R^{2a} is —N(CH₃)₂.

In some embodiments, the invention relates to a compound of formula (I-A-1), or a pharmaceutically acceptable salt thereof, wherein R^{3a} is H, halo, —CN, C₁-C₆ alkyl, C₁-C₆ alkoxy, —NH₂, —N(C₁-C₆ alkyl)₂, —(C₁-C₆ alkylene)-OH, —(C₁-C₆ alkylene)-NH₂, —C(O)(C₁-C₆ alkyl), —C(O)NH₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂)—C(O)NHC(O)CH₃, —NHS(O)₂CH₃, —S(C₁-C₆ alkyl), C₃-C₇ cycloalkyl, C₆-C₁₀ aryl, 4-10 membered heterocyclyl, 4-10 membered heteroaryl, wherein said heterocyclyl or heteroaryl is optionally substituted with 1-2 R^{a′}; and each R^{a′} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₃, cyclopropyl, azetidinyl, or 1-methylazetidinyl. In some embodiments, the invention relates to a compound of formula (I-A-1), or a pharmaceutically acceptable salt thereof, wherein R^{3a} is H, halo, —CN, C₁-C₆ alkyl, C₁-C₆ alkoxy, —NH₂, —N(C₁-C₆ alkyl)₂, —(C₁-C₆ alkylene)-OH, —(C₁-C₆ alkylene)-NH₂, —C(O)(C₁-C₆ alkyl), —C(O)NH₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂). In other embodiments, R^{3a} is halo. In other embodiments, R^{3a} is C₁-C₆ alkyl. In other embodiments, R^{3a} is C₁-C₆ alkoxy. In other embodiments, R^{3a} is —N(C₁-C₆ alkyl)₂. In other embodiments, R^{3a} is —(C₁-C₆ alkylene)-OH. In other embodiments, R^{3a} is —(C₁-C₆ alkylene)-NH₂. In other embodiments, R^{3a} is —C(O)(C₁-C₆ alkyl). In other embodiments, R^{3a} is —C(O)O(C₁-C₆ alkyl). In other embodiments, R^{3a} is —S(O)₂—(C₁-C₆ alkyl). In other embodiments, R^{3a} is —S(O)(NH)(C₁-C₆ alkyl). In other embodiments, R^{3a} is —CH₂C(O)NH(C₁-C₆ alkyl). In other embodiments, R^{3a} is —NHC(O)(C₁-C₆ alkyl). In other embodiments, R^{3a} is —C(O)NHC(O)CH₃. In other embodiments, R^{3a} is —NHS(O)₂CH₃. In other embodiments, R^{3a} is —S(C₁-C₆ alkyl). In other embodiments, R^{3a} is C₃-C₇ cycloalkyl. In other embodiments, R^{3a} is C₆-C₁₀ aryl. In other embodiments, R^{3a} is 4-10 membered heterocyclyl, wherein said heterocyclyl is optionally substituted with 1-2 R^{a′}; and each R^{a} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₃, cyclopropyl, azetidinyl, or 1-methylazetidinyl. In other embodiments, R^{3a} is 4-10 membered heteroaryl, wherein said heteroaryl is optionally substituted with 1-2 R^{a′}; and each R^{a′} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₃, cyclopropyl, azetidinyl, or 1-methylazetidinyl. In some embodiments, the invention relates to a compound of formula (I-A-1), or a pharmaceutically acceptable salt thereof, wherein R^{3a} is H, Cl, Br, —CN, —CH₃, —CH(CH₃)₂, —OCH₃, —NH₂, —N(CH₃)₂, —CH₂OH, —CH(CH₃)(OH)—C(CH₃)₂(OH), —CH₂NH₂, —C(O)CH₃, —C(O)NH₂, —C(O)OCH₂CH₃, —S(O)₂—(CH₃), —S(O)₂CH(CH₃)₂, —S(O)(NH)(CH₃), —S(O)(NH)CH(CH₃)₂, —CH₂C(O)NH₂, —CH₂C(O)NH(CH₃), —NHC(O)(CH₃), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂), —C(O)NHC(O)CH₃, —NHS(O)₂CH₃, —SCH₃, cyclopropyl, phenyl,

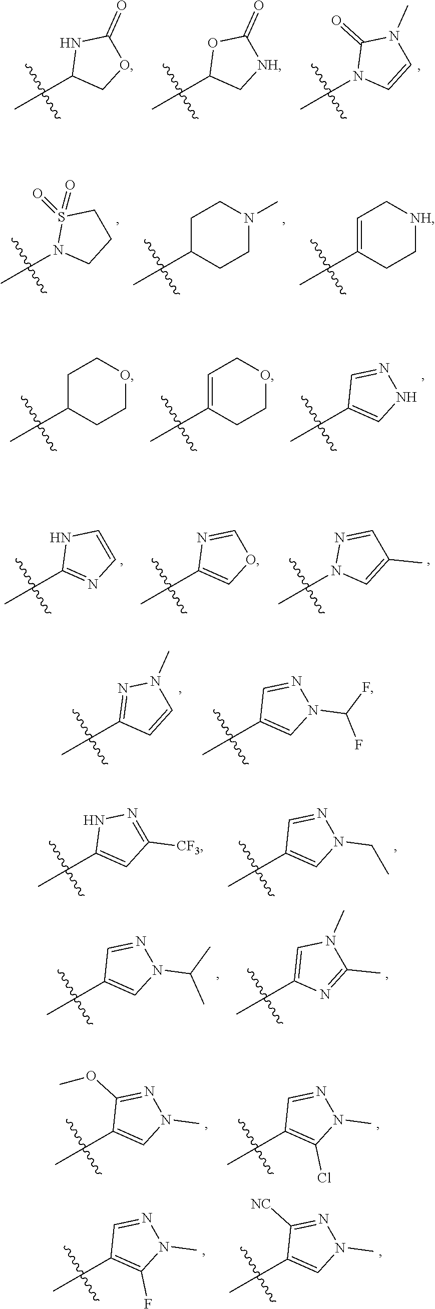

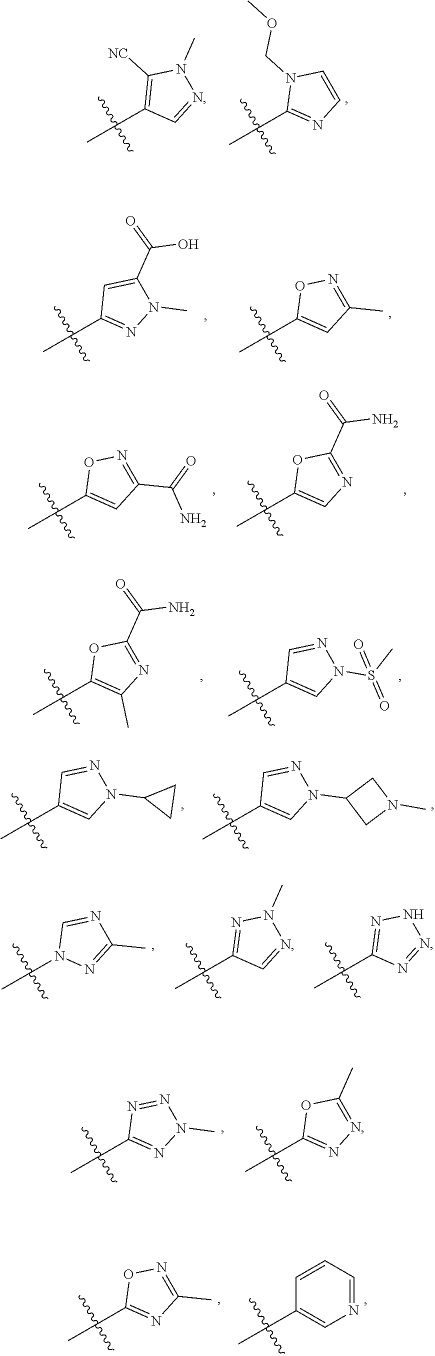

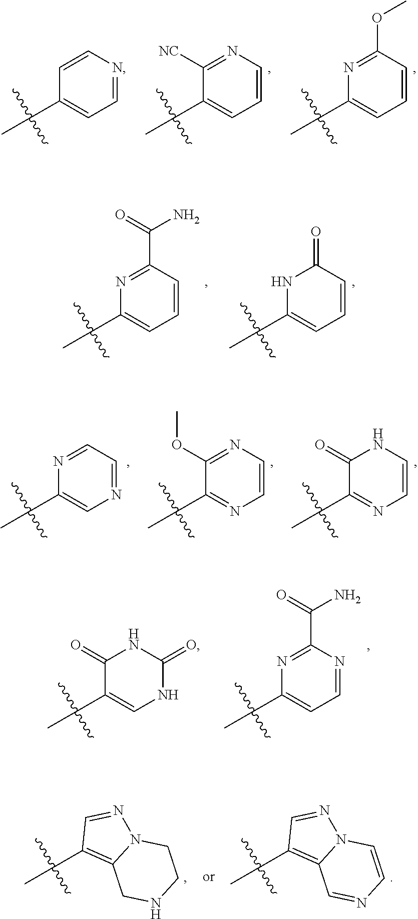

In some embodiments, the invention relates to a compound of formula (I-A-1), or a pharmaceutically acceptable salt thereof, wherein R^{3a} is H, Cl, —CN, —CH₃, —CH(CH₃)₂, —OCH₃, —NH₂, —N(CH₃)₂, —CH₂OH, —CH(CH₃)(OH)—C(CH₃)₂(OH), —CH₂NH₂, —C(O)CH₃, —C(O)NH₂, —C(O)OCH₂CH₃, —S(O)₂—(CH₃), —S(O)(NH)(CH₃), —CH₂C(O)NH₂, —CH₂C(O)NH(CH₃), —NHC(O)(CH₃), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂). In other embodiments, R^{3a} is H. In other embodiments, R^{3a} is Cl. In other embodiments, R^{3a} is —CN. In other embodiments, R^{3a} is —CH₃. In other embodiments, R^{3a} is —CH(CH₃)₂. In other embodiments, R^{3a} is —OCH₃. In other embodiments, R^{3a} is —NH₂. In other embodiments, R^{3a} is —N(CH₃)₂. In other embodiments, R^{3a} is —CH₂OH. In other embodiments, R^{3a} is —CH(CH₃)(OH). In other embodiments, R^{3a} is —C(CH₃)₂(OH). In other embodiments, R^{3a} is —CH₂NH₂. In other embodiments, R^{3a} is —C(O)CH₃. In other embodiments, R^{3a} is —C(O)NH₂. In other embodiments, R^{3a} is —C(O)OCH₂CH₃. In other embodiments, R^{3a} is —S(O)₂—(CH₃). In other embodiments, R^{3a} is —S(O)(NH)(CH₃). In other embodiments, R^{3a} is —CH₂C(O)NH₂. In other embodiments, R^{3a} is —CH₂C(O)NH(CH₃). In other embodiments, R^{3a} is —NHC(O)(CH₃). In other embodiments, R^{3a} is —CH₂NHC(O)CH(CH₃)(NH₂). In other embodiments, R^{3a} is —CH₂N(CH₃)C(O)CH(CH₃)(N(CH₃)₂). In other embodiments, R^{3a} is —CH₂CH₂NHC(O)CH(CH₃)(NH₂). In other embodiments, R^{3a} is —NHC(O)CH(CH₃)(NH₂). In other embodiments, R^{3a} is Br. In other embodiments, R^{3a} is —S(O)₂CH(CH₃)₂. In other embodiments, R^{3a} is —S(O)(NH)CH(CH₃)₂. In other embodiments, R^{3a} is —C(O)NHC(O)CH₃. In other embodiments, R^{3a} is —NHS(O)₂CH₃. In other embodiments, R^{3a} is —SCH₃. In other embodiments, R^{3a} is cyclopropyl. In other embodiments, R^{3a} is phenyl. In other embodiments, R^{3a} is

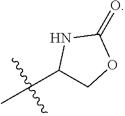 [SMILES: *C1COC(=O)N1]

In other embodiments, R^{3a} is

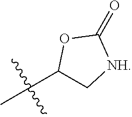 [SMILES: *C1CNC(=O)O1]

In other embodiments, R^{3a} is

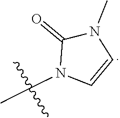 [SMILES: *n1ccn(C)c1=O]

In other embodiments, R^{3a} is

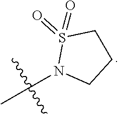 [SMILES: *N1CCCS1(=O)=O]

In other embodiments, R^{3a} is

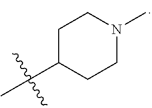 [SMILES: *C1CCN(C)CC1]

In other embodiments, R^{3a} is

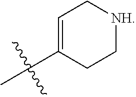 [SMILES: *C1=CCNCC1]

In other embodiments, R^{3a} is

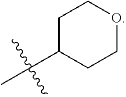 [SMILES: *C1CCOCC1]

In other embodiments, R^{3a} is

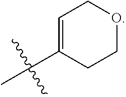 [SMILES: *C1=CCOCC1]

In other embodiments, R^{3a} is

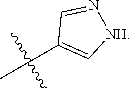 [SMILES: *c1cn[nH]c1]

In other embodiments, R^{3a} is

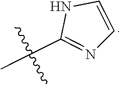 [SMILES: *c1ncc[nH]1]

In other embodiments, R^{3a} is

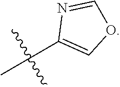 [SMILES: *c1cocn1]

In other embodiments, R^{3a} is

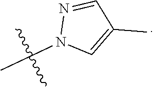 [SMILES: *n1cc(C)cn1]

In other embodiments, R^{3a} is

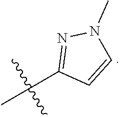 [SMILES: *c1ccn(C)n1]

In other embodiments, R^{3a} is

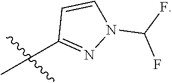 [SMILES: *c1ccn(C(F)F)n1]

In other embodiments, R^{3a} is

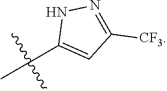 [SMILES: *c1cc(C(F)(F)F)n[nH]1]

In other embodiments. R^{3a} is

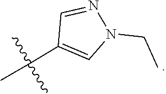 [SMILES: *c1cnn(CC)c1]

In other embodiments, R^{3a} is

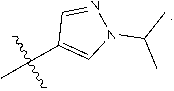 [SMILES: *c1cnn(C(C)C)c1]

In other embodiments, R^{3a} is

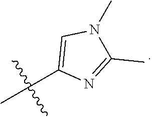 [SMILES: *c1cn(C)c(C)n1 ; "."]

In other embodiments, R^{3a} is

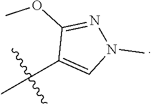

In other embodiments, R^{3a} is

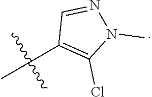 [SMILES: *c1cnn(C)c1Cl]

In other embodiments, R^{3a} is

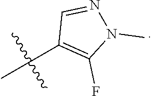 [SMILES: *c1cnn(C)c1F]

In other embodiments, R^{3a} is

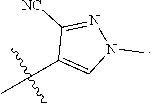 [SMILES: *c1cn(C)nc1C#N]

In other embodiments, R^{3a} is

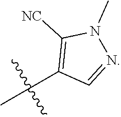 [SMILES: *c1cnn(C)c1C#N]

In other embodiments, R^{3a} is

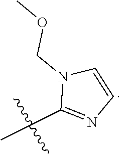 [SMILES: *c1nccn1COC]

In other embodiments, R^{3a} is

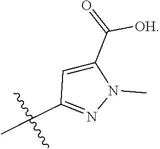 [SMILES: *c1cc(C(=O)O)n(C)n1]

In other embodiments, R^{3a} is

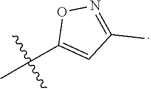 [SMILES: *c1cc(C)no1]

In other embodiments, R^{3a} is

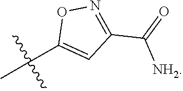 [SMILES: *c1cc(C(N)=O)no1]

In other embodiments, R^{3a} is

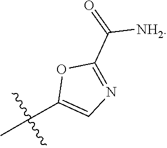 [SMILES: *c1cnc(C(N)=O)o1]

In other embodiments, R^{3a} is

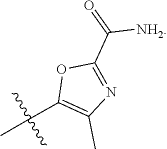 [SMILES: *c1oc(C(N)=O)nc1C]

In other embodiments, R^{3a} is

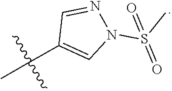 [SMILES: *c1cnn(S(C)(=O)=O)c1]

In other embodiments, R^{3a} is

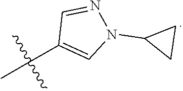 [SMILES: *c1cnn(C2CC2)c1]

In other embodiments, R^{3a} is

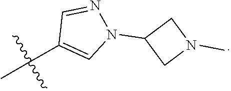 [SMILES: *c1cnn(C2CN(C)C2)c1]

In other embodiments. R^{3a} is

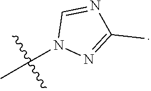 [SMILES: *n1cnc(C)n1]

In other embodiments, R^{3a} is

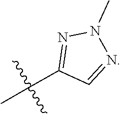 [SMILES: *c1cnn(C)n1]

In other embodiments. R^{3a} is

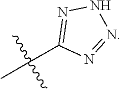 [SMILES: *c1nn[nH]n1]

In other embodiments, R^{3a} is

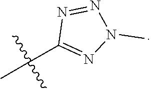 [SMILES: *c1nnn(C)n1]

In other embodiments, R^{3a} is

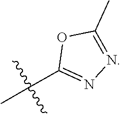 [SMILES: *c1nnc(C)o1]

In other embodiments, R^{3a} is

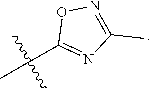 [SMILES: *c1nc(C)no1]

In other embodiments, R^{3a} is

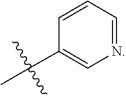 [SMILES: *c1cccnc1]

In other embodiments, R^{3a} is

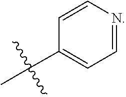 [SMILES: *c1ccncc1]

In other embodiments, R^{3a} is

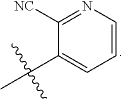 [SMILES: *c1cccnc1C#N]

In other embodiments, R^{3a} is

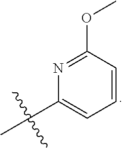 [SMILES: *c1cccc(OC)n1]

In other embodiments, R^{3a} is

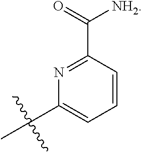 [SMILES: *c1cccc(C(N)=O)n1]

In other embodiments, R^{3a} is

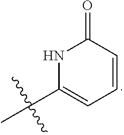 [SMILES: *c1cccc(=O)[nH]1]

In other embodiments, R^{3a} is

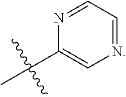 [SMILES: *c1cnccn1]

In other embodiments, R^{3a} is

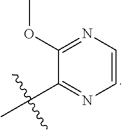 [SMILES: *c1nccnc1OC]

In other embodiments, R^{3a} is

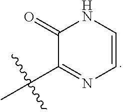 [SMILES: *c1ncc[nH]c1=O]

In other embodiments, R^{3a is}

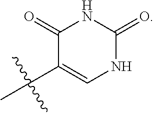 [SMILES: *c1c[nH]c(=O)[nH]c1=O]

In other embodiments, R^{3a} is

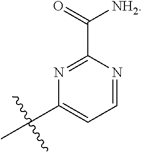 [SMILES: *c1ccnc(C(N)=O)n1]

In other embodiments, R^{3a} is

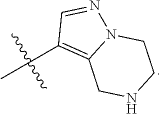 [SMILES: *c1cnn2c1CNCC2]

In other embodiments, R^{3a} is

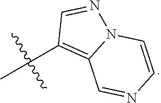 [SMILES: *c1cnn2ccncc12]

In some embodiments, the invention relates to a compound of formula (I-A-1), or a pharmaceutically acceptable salt thereof, wherein R^{3a} is —S(O)R′, —S(O)₂R′, —S(O)(NH)R′, —S(O)(NCH₃)R′, or —S(O)₂NCH₃R′; and R^{2a} and R′, together with the atoms to which they are attached, join together to form a 6-membered heterocyclyl. In other embodiments, R^{3a} is —S(O)R′; and R^{2a} and R′, together with the atoms to which they are attached, join together to form a 6-membered heterocyclyl. In other embodiments, R^{3a} is —S(O)₂R′; and R^{2a} and R′, together with the atoms to which they are attached, join together to form a 6-membered heterocyclyl. In other embodiments, R^{3a} is —S(O)(NH)R′; and R^{2a} and R′, together with the atoms to which they are attached, join together to form a 6-membered heterocyclyl. In other embodiments, R^{3a} is —S(O)(NCH₃)R′; and R^{2a} and R′, together with the atoms to which they are attached, join together to form a 6-membered heterocyclyl. In other embodiments, R^{3a} is —S(O)₂NCH₃R′; and R^{2a} and R′, together with the atoms to which they are attached, join together to form a 6-membered heterocyclyl.

In some embodiments, the invention relates to a compound of formula (I-A-1), or a pharmaceutically acceptable salt thereof, wherein R^{5a} is H, halo, C₁-C₆ alkyl, C₁-C₆ alkoxy, —CH₂OH, —CH₂O(C₁-C₆ alkyl), —C(O)O(C₁-C₆ alkyl), 4-10 membered heteroaryl, wherein said heteroaryl is optionally substituted with R^{a′}; and R^{a′} is —CH₃, —CHF₂, or —CH₂OCH₃. In some embodiments, the invention relates to a compound of formula (I-A-1), or a pharmaceutically acceptable salt thereof, wherein R^{5a} is H, C₁-C₆ alkyl, C₁-C₆ alkoxy, —CH₂OH, —CH₂O(C₁-C₆ alkyl), or —C(O)O(C₁-C₆ alkyl). In other embodiments, R^{5a} is C₁-C₆ alkyl. In other embodiments, R^{5a} is C₁-C₆ alkoxy. In other embodiments, R^{5a} is —CH₂O(C₁-C₆ alkyl). In other embodiments, R^{5a} is —C(O)O(C₁-C₆ alkyl). In other embodiments, R^{5a} is halo. In other embodiments, R^{5a} is 4-10 membered heteroaryl, wherein said heteroaryl is optionally substituted with R^{a′}; and R^{a′} is —CH₃, —CHF₂, or —CH₂OCH₃. In some embodiments, the invention relates to a compound of formula (I-A-1), or a pharmaceutically acceptable salt thereof, wherein R^{5a} is H, Br,

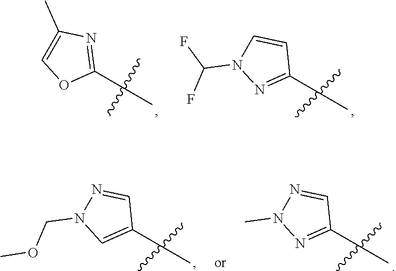

In some embodiments, the invention relates to a compound of formula (I-A-1), or a pharmaceutically acceptable salt thereof, wherein R^{5a} is H, —CH₃, —OCH₃, —OCH₂CH₃, —CH₂OH, —CH₂OCH₃, or —C(O)OCH₂CH₃. In other embodiments, R^{5a} is H. In other embodiments, R^{5a} is —CH₃. In other embodiments, R^{5a} is —OCH₃. In other embodiments, R^{5a} is —OCH₂CH₃. In other embodiments, R^{5a} is —CH₂OH. In other embodiments, R^{5a} is —CH₂OCH₃. In other embodiments, R^{5a} is —C(O)OCH₂CH₃. In other embodiments, R^{5a} is Br. In other embodiments, R^{5a} is

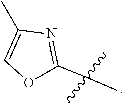 [SMILES: *c1nc(C)co1]

In other embodiments, R^{5a} is

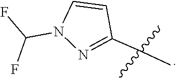 [SMILES: *c1ccn(C(F)F)n1]

In other embodiments, R^{5a} is

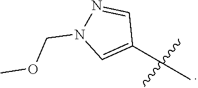 [SMILES: *c1cnn(COC)c1 ; "."]

In other embodiments, R^{5a} is

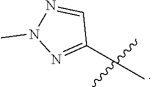 [SMILES: *c1cnn(C)n1 ; "."]

In some embodiments, the invention relates to a compound of formula (I-A-1), or a pharmaceutically acceptable salt thereof, wherein R^{2b} is H, halo, C₁-C₆ alkyl, C₁-C₆ alkoxy, —OH, or —CH₂OH. In other embodiments, R^{2b} is halo. In other embodiments, R^{2b} is C₁-C₆ alkyl. In other embodiments, R^{2b} is C₁-C₆ alkoxy. In some embodiments, the invention relates to a compound of formula (I-A-1), or a pharmaceutically acceptable salt thereof, wherein R^{2b} is H, F, Cl, —CH₃, —CH(CH₃)₂, —C(CH₃)₃, —OCH₃, —OCH₂CH₃, —OH, or —CH₂OH. In other embodiments, R^{2b} is H. In other embodiments, R^{2b} is F. In other embodiments, R^{2b} is Cl. In other embodiments, R^{2b} is —CH₃. In other embodiments, R^{2b} is —CH(CH₃)₂. In other embodiments, R^{2b} is —C(CH₃)₃. In other embodiments, R^{2b} is —OCH₃. In other embodiments, R^{2b} is —OCH₂CH₃. In other embodiments, R^{2b} is —OH. In other embodiments, R^{2b} is —CH₂OH.

In some embodiments, the invention relates to a compound of formula (I-A-1), or a pharmaceutically acceptable salt thereof, wherein R^{3b} is H, halo, C₁-C₆ alkyl, C₁-C₆ haloalkyl, or C₁-C₆ alkoxy. In other embodiments, R^{3b} is halo. In other embodiments, R^{3b} is C₁-C₆ alkyl. In other embodiments, R^{3b} is C₁-C₆ haloalkyl. In other embodiments, R^{3b} is C₁-C₆ alkoxy. In some embodiments, the invention relates to a compound of formula (I-A-1), or a pharmaceutically acceptable salt thereof, wherein R^{3b} is H, F, Cl, —CH₃, —C(CH₃)₃, —CF₃, —OCH₃, —OCH₂CH₃, or —OC(CH₃)₃. In other embodiments, R^{3b} is H. In other embodiments, R^{3b} is F. In other embodiments, R^{3b} is Cl. In other embodiments, R^{3b} is —CH₃. In other embodiments, R^{3b} is —C(CH₃)₃. In other embodiments, R^{3b} is —CF₃. In other embodiments, R^{3b} is —OCH₃. In other embodiments, R^{3b} is —OCH₂CH₃. In other embodiments, R^{3b} is —OC(CH₃)₃.

In some embodiments, the invention relates to a compound of formula (I-A-1), or a pharmaceutically acceptable salt thereof, wherein R^{4b} is H, halo, C₁-C₆ alkyl, C₁-C₆ haloalkyl, C₁-C₆ alkoxy, C₁-C₆ haloalkoxy, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen. In other embodiments, R^{4b} is halo. In other embodiments, R^{4b} is C₁-C₆ alkyl. In other embodiments, R^{4b} is C₁-C₆ haloalkyl. In other embodiments, R^{4b} is C₁-C₆ alkoxy. In other embodiments, R^{4b} is C₁-C₆ haloalkoxy. In other embodiments, R^{4b} is C₃-C₆ cycloalkyl, wherein said cycloalkyl is optionally substituted with one or more halogen. In other embodiments, R^{4b} is (C₁-C₆ alkyl)-(C₃-C₆ cycloalkyl)-, wherein cycloalkyl in said (C₁-C₆ alkyl)-(C₃-C₆ cycloalkyl)- is optionally substituted with one or more halogen. In other embodiments, R^{4b} is (C₁-C₆ haloalkyl)-(C₃-C₆ cycloalkyl)-, wherein cycloalkyl in said (C₁-C₆ haloalkyl)-(C₃-C₆ cycloalkyl)- is optionally substituted with one or more halogen. In some embodiments, the invention relates to a compound of formula (I-A-1), or a pharmaceutically acceptable salt thereof, wherein R^{4b} is H, F, Cl, —CH₃, —CH(CH₃)₂, —C(CH₃)₃, —C(CH₃)₂(CH₂CH₃), —CF₃, —C(CH₃)₂(CF₃), —OCH₃, —OC(CH₃)₃, —OC(CH₃)₃, —OCF₃, cyclopropyl, 1-trifluoromethylcyclopropyl, 3,3-difluorocyclobutyl, or 3,3-difluoro-1-methylcyclobutyl. In other embodiments, R^{4b} is H. In other embodiments, R^{4b} is F. In other embodiments, R^{4b} is Cl. In other embodiments, R^{4b} is —CH₃. In other embodiments, R^{4b} is —CH(CH₃)₂. In other embodiments, R^{4b} is —C(CH₃)₃. In other embodiments, R^{4b} is —C(CH₃)₂(CH₂CH₃). In other embodiments, R^{4b} is —CF₃. In other embodiments, R^{4b} is —C(CH₃)₂(CF₃). In other embodiments. R⁴ is —OCH₃. In other embodiments, R^{4b} is —OC(CH₃)₃. In other embodiments, R^{4b} is —OC(CH₃)₃. In other embodiments, R^{4b} is —OCF₃. In other embodiments, R^{4b} is cyclopropyl. In other embodiments, R^{4b} is 1-trifluoromethylcyclopropyl. In other embodiments, R^{4b} is 3,3-difluorocyclobutyl. In other embodiments, Rib is 3,3-difluoro-1-methylcyclobutyl.

In some embodiments, the invention relates to a compound of formula (I-A-1), or a pharmaceutically acceptable salt thereof, wherein R^{2b} and R^{3b}, together with the carbon atoms to which they are attached, form a ring of formula

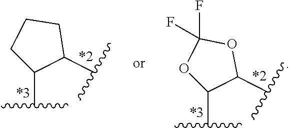 [SMILES: *C1CCCC1* ; *C1OC(F)(F)OC1*]

In some embodiments, the invention relates to a compound of formula (I-A-1), or a pharmaceutically acceptable salt thereof, wherein R^{2b} and R^{3b}, together with the carbon atoms to which they are attached, form a ring of formula

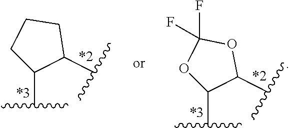 [SMILES: *C1CCCC1* ; *C1OC(F)(F)OC1*]

and R^{4b} is H. In other embodiments, R^{2b} and R^{3b}, together with the carbon atoms to which they are attached, form a ring of formula

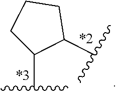 [SMILES: *C1CCCC1*]

In other embodiments, R^{2b} and R^{3b}, together with the carbon atoms to which they are attached, form a ring of formula

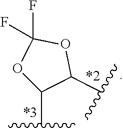 [SMILES: *C1OC(F)(F)OC1*]

In some embodiments, the invention relates to a compound of formula (I-A-1), or a pharmaceutically acceptable salt thereof, wherein R^{3b} and R^{4b}, together with the carbon atoms to which they are attached, form a ring of formula

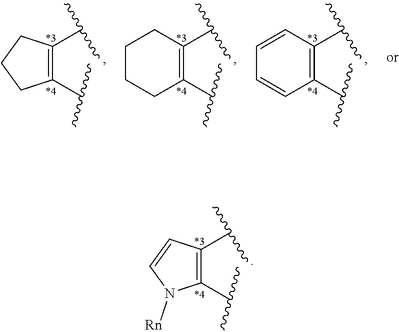

In some embodiments, the invention relates to a compound of formula (I-A-1), or a pharmaceutically acceptable salt thereof, wherein R^{3b} and R^{4b}, together with the carbon atoms to which they are attached, form a ring of formula

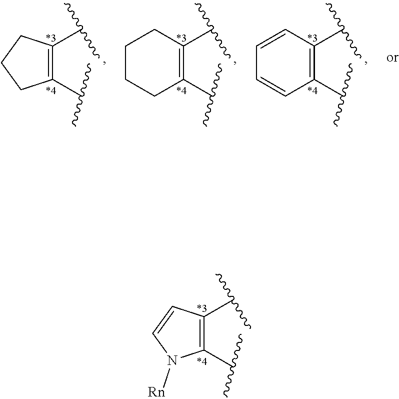

and R^{2b} is H. In other embodiments, R^{3b} and R^{4b}, together with the carbon atoms to which they are attached, form a ring of formula

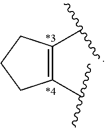 [SMILES: *C1=C(*)CCC1]

In other embodiments, R^{3b} and R^{4b}, together with the carbon atoms to which they are attached, form a ring of formula

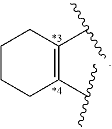 [SMILES: *C1=C(*)CCCC1]

In other embodiments, R^{3b} and R^{4b}, together with the carbon atoms to which they are attached, form a ring of formula

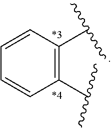 [SMILES: *c1ccccc1*]

In other embodiments, R^{3b} and R^{4b}, together with the carbon atoms to which they are attached, form a ring of formula

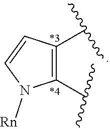

In some embodiments, the invention relates to a compound of formula (I-A-1), or a pharmaceutically acceptable salt thereof, wherein R^{6b} is C₁-C₆ alkyl. In some embodiments, the invention relates to a compound of formula (I-A-1), or a pharmaceutically acceptable salt thereof, wherein R^{6b} is —CH₃.

In some embodiments, the invention relates to a compound of formula (I-A-1), or a pharmaceutically acceptable salt thereof, wherein R^{6b} is H, halo, C₁-C₆ alkyl, or C₁-C₆ alkoxy. In other embodiments, R^{6b} is halo. In other embodiments, R^{6b} is C₁-C₆ alkyl. In other embodiments, R^{6b} is C₁-C₆ alkoxy. In some embodiments, the invention relates to a compound of formula (I-A-1), or a pharmaceutically acceptable salt thereof, wherein R^{6b} is H, Cl, —CH₃, or —OCH₃. In other embodiments, R^{6b} is H. In other embodiments, R^{6b} is Cl. In other embodiments, R^{6b} is —CH₃. In other embodiments, R^{6b} is —OCH₃.

In some embodiments, the invention relates to a compound of formula (I-A-1), or a pharmaceutically acceptable salt thereof, wherein:
X is CR^{5b} and R^{5b} is H, halo, C₁-C₆ alkyl, C₁-C₆ haloalkyl, C₁-C₆ alkoxy, or —C(O)(C₁-C₆ alkyl);
R^{2b} is H, halo, C₁-C₆ alkyl, C₁-C₆ alkoxy, —OH, or —CH₂OH; and
R^{4b} is H, halo, C₁-C₆ alkyl, C₁-C₆ haloalkyl, C₁-C₆ alkoxy, C₁-C₆ haloalkoxy, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-A-2):

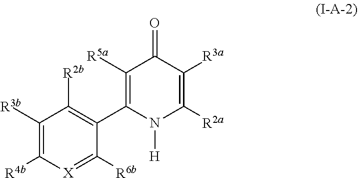

or a pharmaceutically acceptable salt thereof, wherein:
   X is CR^{5b} or N;
   R^{2a} is H, C₁-C₆ alkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
   R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂);
   R^{5a} is H, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl); R^{2b}, R^{3b}, and R^{4b} are defined as follows:
   (i) R^{2b} is H, halo, C₁-C₆ alkyl, C₁-C₆ alkoxy, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
      R^{3b} is H, halo, C₁-C₆ alkyl, C₁-C₆ haloalkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
      R^{4b} is C₂-C₆ alkyl, C₂-C₆ haloalkyl, —OH, —CH₂OH, —C(O)(C₁-C₆ alkyl), —C(O)(C₁-C₆ haloalkyl), C₄-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₄-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; or
   (ii) R^{2b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
   R^{3b} and R^{4b}, together with the carbon atoms to which they are attached, form a ring of formula:

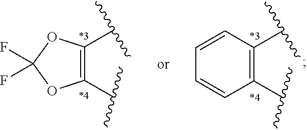 [SMILES: *C1=C(*)OC(F)(F)O1 ; *c1ccccc1*]

R^{5b} is H, halo, C₁-C₆ alkyl, C₁-C₆ haloalkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
   R^{6b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen,
provided that:
   (i) if X is N, then R^{3b} and R^{4b}, together with the carbon atoms to which they are attached, form a ring of formula:

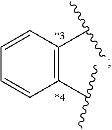 [SMILES: *c1ccccc1*]

and
(ii) if R^{2a} is H, then R^{3a} is halo, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
(iii) no more than one of R^{2b}, R^{3b}, R^{5b}, and R^{6b} is chloro.

In some embodiments, the invention relates to a compound of formula (I-A-2), or a pharmaceutically acceptable salt thereof, wherein X is N. In some embodiments, the invention relates to a compound of formula (I-A-2), or a pharmaceutically acceptable salt thereof, wherein X is CR^{5b}. In some embodiments, the invention relates to a compound of formula (I-A-2), or a pharmaceutically acceptable salt thereof, wherein X is CR^{5b} and R^{5b} is H, halo, C₁-C₆ alkyl, or C₁-C₆ haloalkyl. In other embodiments, R^{5b} is halo. In other embodiments, R^{5b} is C₁-C₆ alkyl. In other embodiments, R^{5b} is C₁-C₆ haloalkyl. In some embodiments, the invention relates to a compound of formula (I-A-2), or a pharmaceutically acceptable salt thereof, wherein X is CR^{5b} and R^{5b} is H, F, Cl, —CH₃, or —CF₃. In other embodiments, R^{5b} is H. In other embodiments, R^{5b} is F. In other embodiments, R^{5b} is Cl. In other embodiments, R^{5b} is —CH₃. In other embodiments, R^{5b} is —CF₃.

In some embodiments, the invention relates to a compound of formula (I-A-2), or a pharmaceutically acceptable salt thereof, wherein R^{2a} is H, C₁-C₆ alkyl, C₁-C₆ alkoxy, or —N(C₁-C₆ alkyl)₂. In other embodiments, R^{2a} is C₁-C₆ alkyl. In other embodiments, R^{2a} is C₁-C₆ alkoxy. In other embodiments, R^{2a} is —N(C₁-C₆ alkyl)₂. In some embodiments, the invention relates to a compound of formula (I-A-2), or a pharmaceutically acceptable salt thereof, wherein R^{2a} is H, —CH₃, —OCH₃, or —N(CH₃)₂. In other embodiments, R^{2a} is H. In other embodiments, R^{2a} is —CH₃. In other embodiments, R^{2a} is —OCH₃. In other embodiments, R^{2a} is —N(CH₃)₂.

In some embodiments, the invention relates to a compound of formula (I-A-2), or a pharmaceutically acceptable salt thereof, wherein R^{3a} is H, halo, —CN, C₁-C₆ alkyl, C₁-C₆ alkoxy, —NH₂, —N(C₁-C₆ alkyl)₂, —(C₁-C₆ alkylene)-OH, —(C₁-C₆ alkylene)-NH₂, —C(O)(C₁-C₆ alkyl), —C(O)NH₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂). In other embodiments. R^{3a} is halo. In other embodiments, R^{3a} is C₁-C₆ alkyl. In other embodiments, R^{3a} is C₁-C₆ alkoxy. In other embodiments, R^{3a} is —N(C₁-C₆ alkyl)₂. In other embodiments, R^{3a} is —(C₁-C₆ alkylene)-OH. In other embodiments, R^{3a} is —(C₁-C₆ alkylene)-NH₂. In other embodiments, R^{3a} is —C(O)(C₁-C₆ alkyl). In other embodiments, R^{3a} is —C(O)O(C₁-C₆ alkyl). In other embodiments, R^{3a} is —S(O)₂—(C₁-C₆ alkyl). In other embodiments, R^{3a} is —S(O)(NH)(C₁-C₆ alkyl). In other embodiments, R^{3a} is —CH₂C(O)NH(C₁-C₆ alkyl). In other embodiments, R^{3a} is —NHC(O)(C₁-C₆ alkyl). In some embodiments, the invention relates to a compound of formula (I-A-2), or a pharmaceutically acceptable salt thereof, wherein R^{3a} is H, Cl, —CN, —CH₃, —CH(CH₃)₂, —OCH₃, —NH₂, —N(CH₃)₂, —CH₂OH, —CH(CH₃)(OH), —C(CH₃)₂(OH), —CH₂NH₂, —C(O)CH₃, —C(O)NH₂, —C(O)OCH₂CH₃, —S(O)₂—CH₃, —S(O)(NH)(CH₃), —CH₂C(O)NH₂, —CH₂C(O)NH(CH₃), —NHC(O)(CH₃), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂). In other embodiments, R^{3a} is H. In other embodiments, R^{3a} is Cl. In other embodiments, R^{3a} is —CN. In other embodiments, R^{3a} is —CH₃. In other embodiments, R^{3a} is —CH(CH₃)₂. In other embodiments, R^{3a} is —OCH₃. In other embodiments, R^{3a} is —NH₂. In other embodiments, R^{3a} is —N(CH₃)₂. In other embodiments, R^{3a} is —CH₂OH. In other embodiments, R^{3a} is —CH(CH₃)(OH). In other embodiments, R^{3a} is —C(CH₃)₂(OH). In other embodiments, R^{3a} is —CH₂NH₂. In other embodiments, R^{3a} is —C(O)CH₃. In other embodiments, R^{3a} is —C(O)NH₂. In other embodiments, R^{3a} is —C(O)OCH₂CH₃. In other embodiments, R^{3a} is —S(O)₂—CH₃. In other embodiments, R^{3a} is —S(O)(NH)(CH₃). In other embodiments, R^{3a} is —CH₂C(O)NH₂. In other embodiments, R^{3a} is —CH₂C(O)NH(CH₃). In other embodiments, R^{3a} is —NHC(O)(CH₃). In other embodiments, R^{3a} is —CH₂NHC(O)CH(CH₃)(NH₂). In other embodiments, R^{3a} is —CH₂N(CH₃)C(O)CH(CH₃)(N(CH₃)₂). In other embodiments, R^{3a} is —CH₂CH₂NHC(O)CH(CH₃)(NH₂). In other embodiments, R^{3a} is —NHC(O)CH(CH₃)(NH₂).

In some embodiments, the invention relates to a compound of formula (I-A-2), or a pharmaceutically acceptable salt thereof, wherein R^{5a} is H, C₁-C₆ alkyl, C₁-C₆ alkoxy, —CH₂OH, —CH₂O(C₁-C₆ alkyl), or —C(O)O(C₁-C₆ alkyl). In other embodiments, R^{5a} is C₁-C₆ alkyl. In other embodiments, R^{5a} is C₁-C₆ alkoxy. In other embodiments, R^{5a} is —CH₂O(C₁-C₆ alkyl). In other embodiments, R^{5a} is —C(O)O(C₁-C₆ alkyl). In some embodiments, the invention relates to a compound of formula (I-A-2), or a pharmaceutically acceptable salt thereof, wherein R^{5a} is H, —CH₃, —OCH₃, —OCH₂CH₃, —CH₂OH, —CH₂OCH₃, or —C(O)OCH₂CH₃. In other embodiments, R^{5a} is H. In other embodiments, R^{5a} is —CH₃. In other embodiments, R^{5a} is —OCH₃. In other embodiments, R^{5a} is —OCH₂CH₃. In other embodiments, R^{5a} is —CH₂OH. In other embodiments, R^{5a} is —CH₂OCH₃. In other embodiments, R^{5a} is —C(O)OCH₂CH₃.

In some embodiments, the invention relates to a compound of formula (I-A-2), or a pharmaceutically acceptable salt thereof, wherein R^{2b} is H, halo, C₁-C₆ alkyl, or C₁-C₆ alkoxy. In other embodiments, R^{2b} is halo. In other embodiments, R^{2b} is C₁-C₆ alkyl. In other embodiments, R² is C₁-C₆ alkoxy. In some embodiments, the invention relates to a compound of formula (I-A-2), or a pharmaceutically acceptable salt thereof, wherein R^{2b} is H, F, —CH₃, —CH(CH₃)₂, or —OCH₃. In other embodiments, R^{2b} is H. In other embodiments, R^{2b} is F. In other embodiments, R^{2b} is —CH₃. In other embodiments, R^{2b} is —CH(CH₃)₂. In other embodiments, R²¹ is —OCH₃.

In some embodiments, the invention relates to a compound of formula (I-A-2), or a pharmaceutically acceptable salt thereof, wherein R^{3b} is H or C₁-C₆ alkyl. In other embodiments, R^{3b} is C₁-C₆ alkyl. In some embodiments, the invention relates to a compound of formula (I-A-2), or a pharmaceutically acceptable salt thereof, wherein R^{3b} is H or —CH₃. In other embodiments, R^{3b} is H. In other embodiments, R^{3b} is —CH₃.

In some embodiments, the invention relates to a compound of formula (I-A-2), or a pharmaceutically acceptable salt thereof, wherein R^{4b} is C₂-C₆ alkyl, C₂-C₆ haloalkyl, C₄-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₄-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen. In other embodiments, R^{4b} is C₂-C₆ alkyl. In other embodiments, R^{4b} is C₂-C₆ haloalkyl. In other embodiments, R^{4b} is C₄-C₆ cycloalkyl, wherein said cycloalkyl is optionally substituted with one or more halogen. In other embodiments, R^{4b} is (C₁-C₆ alkyl)-(C₃-C₆ cycloalkyl)-, wherein cycloalkyl in said (C₁-C₆ alkyl)-(C₃-C₆ cycloalkyl)- is optionally substituted with one or more halogen. In other embodiments, R^{4b} is (C₁-C₆ haloalkyl)-(C₃-C₆ cycloalkyl)-, wherein cycloalkyl in said (C₁-C₆ haloalkyl)-(C₃-C₆ cycloalkyl)- is optionally substituted with one or more halogen. In some embodiments, the invention relates to a compound of formula (I-A-2), or a pharmaceutically acceptable salt thereof, wherein R^{4b} is —CH(CH₃)₂, —C(CH₃)₃, —C(CH₃)₂(CH₂CH₃), —C(CH₃)₂(CF₃), 1-trifluoromethylcyclopropyl, 3,3-difluorocyclobutyl, or 3,3-difluoro-1-methylcyclobutyl. In other embodiments, R^{4b} is —CH(CH₃)₂. In other embodiments, R^{4b} is —C(CH₃)₃. In other embodiments, R^{4b} is —C(CH₃)₂(CH₂CH₃). In other embodiments, R^{4b} is —C(CH₃)₂(CF₃). In other embodiments, R^{4b} is 1-trifluoromethylcyclopropyl. In other embodiments, R^{4b} is 3,3-difluorocyclobutyl. In other embodiments, R^{4b} is 3,3-difluoro-1-methylcyclobutyl.

In some embodiments, the invention relates to a compound of formula (I-A-2), or a pharmaceutically acceptable salt thereof, wherein R^{3b} and R^{4b}, together with the carbon atoms to which they are attached, form a ring of formula

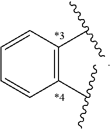 [SMILES: *c1ccccc1*]

In some embodiments, the invention relates to a compound of formula (I-A-2), or a pharmaceutically acceptable salt thereof, wherein R^{3b} and R^{4b}, together with the carbon atoms to which they are attached, form a ring of formula

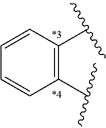 [SMILES: *c1ccccc1*]

### and R^{2b} is H.

In some embodiments, the invention relates to a compound of formula (I-A-2), or a pharmaceutically acceptable salt thereof, wherein R^{6b} is H, C₁-C₆ alkyl, or C₁-C₆ alkoxy. In other embodiments, R^{6b} is C₁-C₆ alkyl. In other embodiments, R^{6b} is C₁-C₆ alkoxy. In some embodiments, the invention relates to a compound of formula (I-A-2), or a pharmaceutically acceptable salt thereof, wherein R^{6b} is H. —CH₃, or —OCH₃. In other embodiments, R^{6b} is H. In other embodiments, R^{6b} is —CH₃. In other embodiments, R^{6b} is —OCH₃.

In some embodiments, the invention relates to a compound of formula (I-A-2), or a pharmaceutically acceptable salt thereof, wherein:
X is CR^{5b} and R^{5b} is H, halo, C₁-C₆ alkyl, or C₁-C₆ haloalkyl;
R^{2b} is H, halo, C₁-C₆ alkyl, or C₁-C₆ alkoxy; and
R^{4b} is C₂-C₆ alkyl, C₂-C₆ haloalkyl, C₄-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₄-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-A-2), or a pharmaceutically acceptable salt thereof, wherein:
R^{5b} is H, F, Cl, —CH₃, or —CF₃;
R^{2b} is H, F, —CH₃, —CH(CH₃)₂, or —OCH₃;
R^{4b} is —CH(CH₃)₂, —C(CH₃)₃, —C(CH₃)₂(CH₂CH₃), —C(CH₃)₂(CF₃), 1-trifluoromethylcyclopropyl, 3,3-difluorocyclobutyl, or 3,3-difluoro-1-methylcyclobutyl.

In some embodiments, the invention relates to a compound of formula (I-B-1):

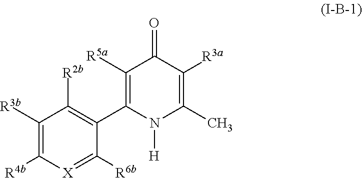

or a pharmaceutically acceptable salt thereof, wherein:
   X is CR^{5b} or N;
   R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂), —C(O)NHC(O)(C₁-C₆ alkyl), —NHS(O)₂CH₃, —S(C₁-C₆ alkyl), C₃-C₇ cycloalkyl, C₆-C₁₀ aryl, 4-10 membered heterocyclyl, 4-10 membered heteroaryl, wherein said heterocyclyl or heteroaryl is optionally substituted with 1-2 R^{a′};
   R^{5a} is H, halo, C₁-C₆ alkyl, C₁-C₆ haloalkyl, C₁-C₆ alkoxy, C₁-C₆ haloalkoxy; —CH₂OH, —CH₂O(C₁-C₆ alkyl), —CH₂O(C₁-C₆ haloalkyl), —C(O)O(C₁-C₆ alkyl), —C(O)O(C₁-C₆ haloalkyl), or 4-10 membered heteroaryl, wherein said heteroaryl is optionally substituted with R^{a′};
   each R^{a′} is independently halo, —CN, —OH, oxo, C₁-C₆ alkyl, C₁-C₆ haloalkyl, C₁-C₆ alkoxy, —(C₁-C₆ alkylene)-(C₁-C₆ alkoxy), —C(O)NH₂, —C(O)OH, —S(O)₂(C₁-C₆ alkyl), C₃-C₇ cycloalkyl, 4-10 membered heterocyclyl, or -(4-10 membered heterocyclyl)-(C₁-C₆ alkyl);
R^{2b}, R^{3b}, and R^{4b} are defined as follows:
   (i) R^{2b} is H, halo. C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
      R^{3b} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
      R^{4b} is H, halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; or
   (ii) R^{2b} and R^{3b}, together with the carbon atoms to which they are attached, form a ring of formula:

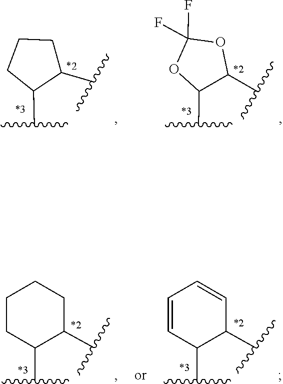 [SMILES: *C1CCCC1* ; *C1OC(F)(F)OC1* ; *C1CCCCC1* ; *C1C=CC=CC1*]

and
   R^{4b} is H, halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; or
(iii) R^{2b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
   R^{3b} and R^{4b}, together with the carbon atoms to which they are attached, form a ring of formula:

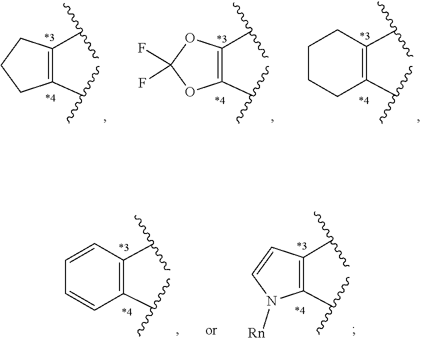

R^{5b} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
R^{6b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
Rₐ is H or C₁-C₆ alkyl,
provided that:
   (i) if X is N, then R^{3b} and R^{4b}, together with the carbon atoms to which they are attached, form a ring of formula:

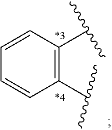 [SMILES: *c1ccccc1*]

and
(ii) if R^{3a} is —C(O)O(C₁-C₂ alkyl), then R^{4b} is H, fluoro, chloro, C₁-C₆ alkyl, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(iii) no more than four of R^{2b}, R^{3b}, R^{4b}, R^{5b}, and R^{6b} are H; and
(iv) no more than one of R^{2b}, R^{3b}, R^{4b}, R^{5b}, and R^{6b} is fluoro; and
(v) no more than one of R^{2b}, R^{3b}, R^{4b}, R^{5b}, and R^{6b} is chloro; and
(vi) if any of R^{2b}, R^{3b}, R^{4b}, R^{5b}, and R^{6b} are halo, then no more than three of R^{2b}, R^{3b}, R^{4b}, R^{5b}, and R^{6b} are H; and
(vii) if R^{2b} or R is —OCH₃ and R^{3a} is H, then no more than three of R^{2b}, R^{3b}, R^{4b}, R^{5b}, and R^{6b} are H; and
(viii) if R^{3b} is fluoro, then R^{6b} is H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(ix) if R^{5b} is fluoro, then R^{2b} is H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(x) if R^{3b} or R^{5b} is —CH₃, then no more three of R^{2b}, R^{3b}, R^{4b}, R^{5b}, and R^{6b} are H; and
(xi) if R^{3b} or R^{5b} is C₁ haloalkyl, then no more than three of R^{2b}, R^{3b}, R^{4b}, R^{5b}, and R^{6b} are H; and
(xii) if R^{3b} or R^{5b} is —OCH₃, then no more than three of R^{2b}, R^{3b}, R^{4b}, R^{5b}, and R^{6b} are H; and
(xiii) if R^{4b} is C₁ haloalkyl, then no more than three of R^{2b}, R^{3b}, R^{5b} and R^{6b} are H; and
(xiv) if R^{4b} is —OCH₃, then no more than three of R^{2b}, R^{3b}, R^{5b} and R^{6b} are H; and
(xv) if R^{4b} is C₁ haloalkoxy, then no more than three of R^{2b}, R^{3b}, R^{5b} and R^{6b} are H; and
(xvi) if R^{4b} is chloro or bromo, then R^{2b} and R^{6b} are each independently H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(xvii) if R^{4b} and R^{3b} are each —OCH₃, then no more than two of R^{2b}, R^{5b}, and R^{6b} are H; and
(xviii) if R^{4b} and R are each —OCH₃, then no more than two of R^{2b}, R^{3b}, and R^{6b} are H.

In some embodiments, the invention relates to a compound of formula (I-B-1), or a pharmaceutically acceptable salt thereof, wherein:
R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
R^{5a} is H, C₁-C₆ alkyl, C₁-C₆ haloalkyl, C₁-C₆ alkoxy, C₁-C₆ haloalkoxy; —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl).

In some embodiments, the invention relates to a compound of formula (I-B-1), or a pharmaceutically acceptable salt thereof, wherein X is N. In some embodiments, the invention relates to a compound of formula (I-B-1), or a pharmaceutically acceptable salt thereof, wherein X is CR^{5b}. In some embodiments, the invention relates to a compound of formula (I-B-1), or a pharmaceutically acceptable salt thereof, wherein X is CR^{5b} and R^{5b} is H, halo, C₁-C₆ alkyl, C₁-C₆ haloalkyl, C₁-C₆ alkoxy, or —C(O)(C₁-C₆ alkyl). In other embodiments, R^{5b} is halo. In other embodiments, R^{5b} is C₁-C₆ alkyl. In other embodiments, R is C₁-C₆ haloalkyl. In other embodiments, R^{5b} is C₁-C₆ alkoxy. In other embodiments, R^{5b} is —C(O)(C₁-C₆ alkyl). In some embodiments, the invention relates to a compound of formula (I-B-1), or a pharmaceutically acceptable salt thereof, wherein X is CR^{5b} and R^{5b} is H, F, Cl, —CH₃, —C(CH₃)₃, —CF₃, —OCH₃, or —C(O)CH₃. In other embodiments, R^{5b} is H. In other embodiments, R^{5b} is F. In other embodiments, R^{5b} is Cl. In other embodiments, R^{5b} is —CH₃. In other embodiments, R^{5b} is —C(CH₃)₃. In other embodiments, R^{5b} is —CF₃. In other embodiments, R^{5b} is —OCH₃. In other embodiments, R^{5b} is —C(O)CH₃.

In some embodiments, the invention relates to a compound of formula (I-B-1), or a pharmaceutically acceptable salt thereof, wherein R^{3a} is H, halo, —CN, C₁-C₆ alkyl, C₁-C₆ alkoxy, —NH₂, —N(C₁-C₆ alkyl)₂, —(C₁-C₆ alkylene)-OH, —(C₁-C₆ alkylene)-NH₂, —C(O)(C₁-C₆ alkyl), —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂), —C(O)NHC(O)CH₃, —NHS(O)₂CH₃, —S(C₁-C₆ alkyl), C₃-C₂ cycloalkyl, C₆-C₁₀ aryl, 4-10 membered heterocyclyl, 4-10 membered heteroaryl, wherein said heterocyclyl or heteroaryl is optionally substituted with 1-2 R^{a′}; and each R^{a′} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₃, cyclopropyl, azetidinyl, or 1-methylazetidinyl. In some embodiments, the invention relates to a compound of formula (I-B-1), or a pharmaceutically acceptable salt thereof, wherein R^{3a} is H, halo, —CN, C₁-C₆ alkyl, C₁-C₆ alkoxy, —NH₂, —N(C₁-C₆ alkyl)₂, —(C₁-C₆ alkylene)-OH, —(C₁-C₆ alkylene)-NH₂, —C(O)(C₁-C₆ alkyl), —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₂)(NH₂), or —NHC(O)CH(CH₃)(NH₂). In other embodiments, R^{3a} is halo. In other embodiments, R^{3a} is C₁-C₆ alkyl. In other embodiments, R^{3a} is C₁-C₆ alkoxy. In other embodiments, R^{3a} is —N(C₁-C₆ alkyl)₂. In other embodiments, R^{3a} is —(C₁-C₆ alkylene)-OH. In other embodiments, R^{3a} is —(C₁-C₆ alkylene)-NH₂. In other embodiments, R^{3a} is —C(O)(C₁-C₆ alkyl). In other embodiments, R^{3a} is —C(O)O(C₁-C₆ alkyl). In other embodiments, R^{3a} is —S(O)₂—(C₁-C₆ alkyl). In other embodiments, R^{3a} is —S(O)(NH)(C₁-C₆ alkyl). In other embodiments, R^{3a} is —CH₂C(O)NH(C₁-C₆ alkyl). In other embodiments, R^{3a} is —NHC(O)(C₁-C₆ alkyl). In other embodiments, R^{3a} is —C(O)NHC(O)CH₃. In other embodiments, R^{3a} is —NHS(O)₂CH₃. In other embodiments, R^{3a} is —S(C₁-C₆ alkyl). In other embodiments, R^{3a} is C₃-C₇ cycloalkyl. In other embodiments, R^{3a} is C₆-C₁₀ aryl. In other embodiments, R^{3a} is 4-10 membered heterocyclyl, wherein said heterocyclyl is optionally substituted with 1-2 R^{a′}; and each R^{a} is independently F, Cl, —CN, —OH, oxo, —CH₃, —CH₂CH₃, —CH(CH₃)₂, —CHF₂, —CF₃. —OCH₃, —CH₂OCH₃, —C(O)NH₂, —C(O)OH, —S(O)₂CH₃, cyclopropyl, azetidinyl, or 1-methylazetidinyl. In other embodiments, R^{3a} is 4-10 membered heteroaryl, wherein said heteroaryl is optionally substituted with 1-2 R^{a′}; and each R^{a′} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₃, cyclopropyl, azetidinyl, or 1-methylazetidinyl. In some embodiments, the invention relates to a compound of formula (I-B-1), or a pharmaceutically acceptable salt thereof, wherein R^{3a} is H, Cl, Br, —CN, —CH₃, —CH(CH₃)₂, —OCH₃, —NH₂, —N(CH₃)₂, —CH₂OH, —CH(CH₃)(OH), —C(CH₃)₂(OH), —CH₂NH₂, —C(O)CH₃, —C(O)OCH₂CH₃, —S(O)₂—(CH₃), —S(O)₂CH(CH₃)₂, —S(O)(NH)(CH₃), —S(O)(NH)CH(CH₃)₂, —CH₂C(O)NH₂, —CH₂C(O)NH(CH₃), —NHC(O)CH₃, —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂)—C(O)NHC(O)CH₃, —NHS(O)₂CH₃, —SCH₃, cyclopropyl, phenyl,

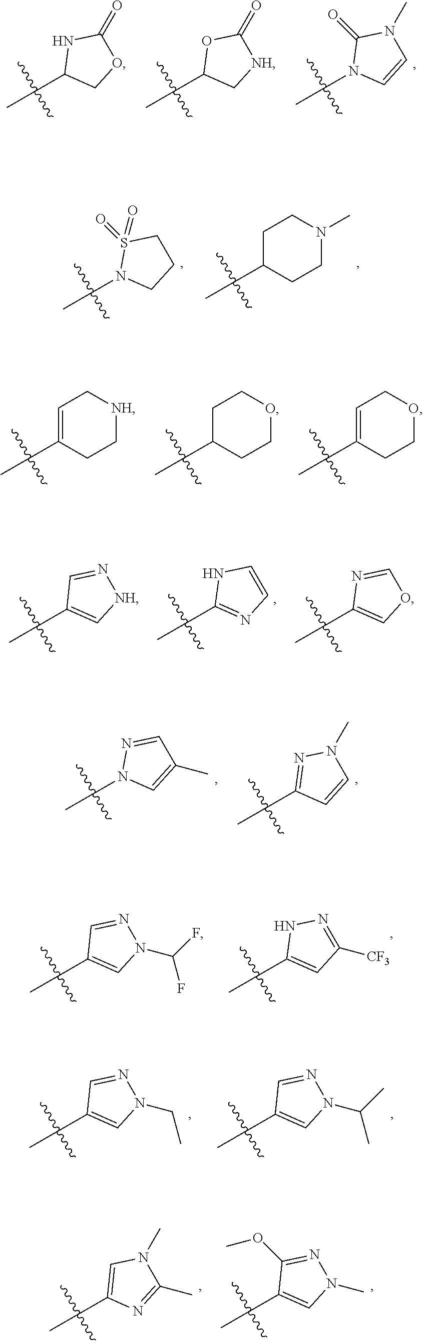

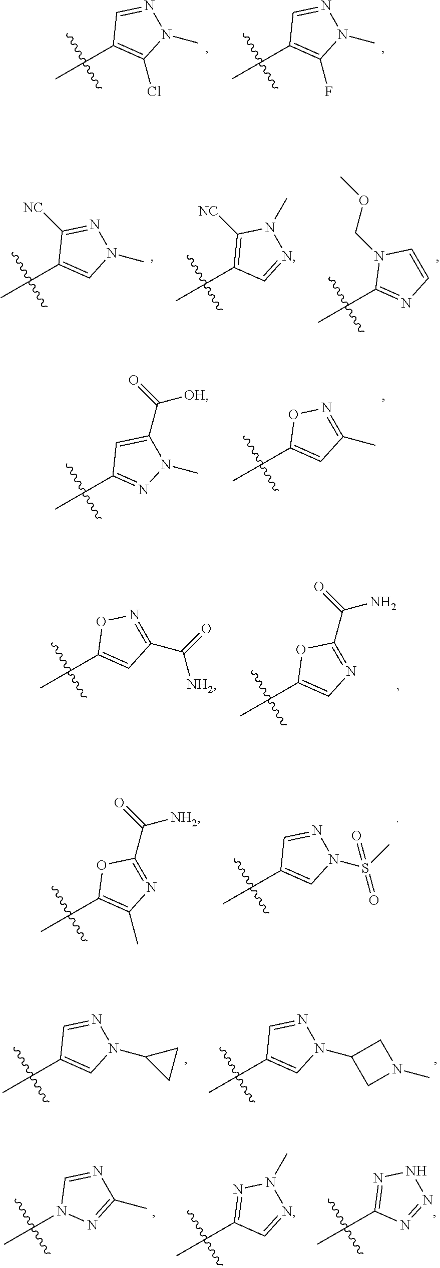

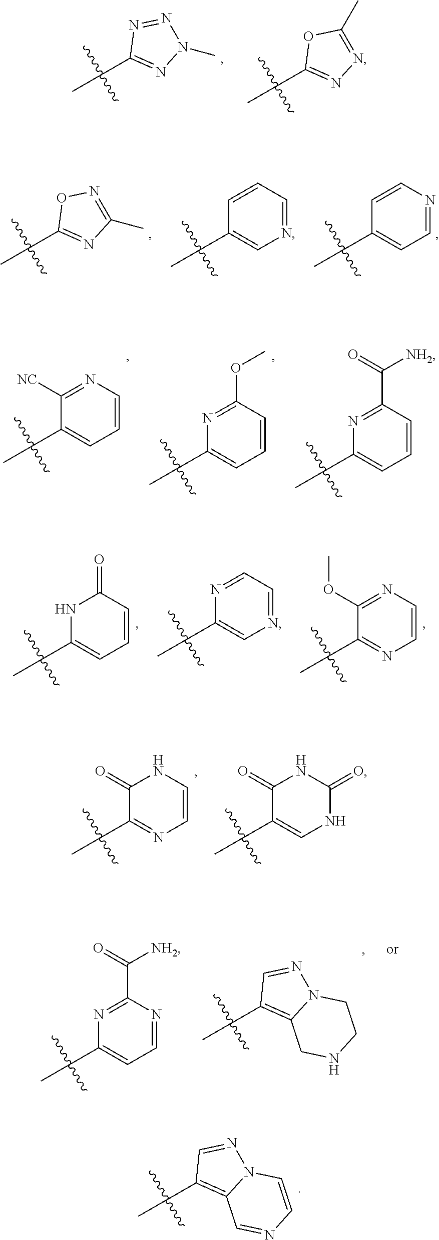

In some embodiments, the invention relates to a compound of formula (I-B-1), or a pharmaceutically acceptable salt thereof, wherein R³ is H, Cl, —CN, —CH, —CH(CH₃)₂, —OCH₃, —NH₂, —N(CH₃)₂, —CH₂OH, —CH(CH₃)(OH), —C(CH₃)₂(OH), —CH₂NH₂, —C(O)CH, —C(O)OCH₂CH₃, —S(O)₂—(CH₃), —S(O)(NH)(CH₃), —CH₂C(O)NH₂, —CH₂C(O)NH(CH₃), —NHC(O)CH₃, —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂). In other embodiments, R^{3a} is H. In other embodiments, R^{3a} is Cl. In other embodiments, R^{3a} is —CN. In other embodiments, R^{3a} is —CH₃. In other embodiments, R^{3a} is —CH(CH₃)₂. In other embodiments, R^{3a} is —OCH₃. In other embodiments, R^{3a} is —NH₂. In other embodiments, R^{3a} is —N(CH₃)₂. In other embodiments, R^{3a} is —CH₂OH. In other embodiments, R^{3a} is —CH(CH₃)(OH). In other embodiments, R^{3a} is —C(CH₄)₂OH). In other embodiments, R^{3a} is —CH₂NH₂. In other embodiments, R^{3a} is —C(O)CH₃. In other embodiments, R^{3a} is —C(O)OCH₂CH₃. In other embodiments, R^{3a} is —S(O)₂—(CH₃). In other embodiments, R^{3a} is —S(O)(NH)(CH₃). In other embodiments, R^{3a} is —CH₂C(O)NH₂. In other embodiments, R^{3a} is —CH₂C(O)NH(CH₃). In other embodiments, R^{3a} is —NHC(O)CH₃. In other embodiments, R^{3a} is —CH₂NHC(O)CH(CH₃)(NH₂). In other embodiments, R^{3a} is —CH₂N(CH₃)C(O)CH(CH₃)(N(CH₃)₂). In other embodiments, R^{3a} is —CH₂CH₂NHC(O)CH(CH₃)(NH₂). In other embodiments, R^{3a} is —NHC(O)CH(CH₃)(NH₂). In other embodiments, R^{3a} is Br. In other embodiments, R^{3a} is —S(O)₂CH(CH₃)₂. In other embodiments, R^{3a} is —S(O)(NH)CH(CH₃)₂. In other embodiments, R^{3a} is —C(O)NHC(O)CH₃. In other embodiments, R^{3a} is —NHS(O)₂CH₃. In other embodiments, R^{3a} is —SCH₃. In other embodiments, R^{3a} is cyclopropyl. In other embodiments, R^{3a} is phenyl. In other embodiments, R^{3a} is

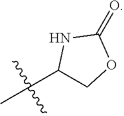 [SMILES: *C1COC(=O)N1]

In other embodiments, R^{3a} is

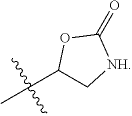 [SMILES: *C1CNC(=O)O1]

In other embodiments, R^{3a} is

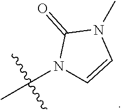 [SMILES: *n1ccn(C)c1=O]

In other embodiments, R^{3a} is

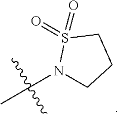 [SMILES: *N1CCCS1(=O)=O]

In other embodiments, R^{3a} is

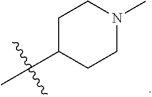 [SMILES: *C1CCN(C)CC1]

In other embodiments, R^{3a} is

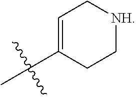 [SMILES: *C1=CCNCC1]

In other embodiments, R^{3a} is

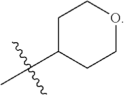 [SMILES: *C1CCOCC1]

In other embodiments, R^{3a} is

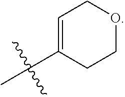 [SMILES: *C1=CCOCC1]

In other embodiments, R^{3a} is

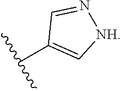 [SMILES: *c1cn[nH]c1]

In other embodiments, R^{3a} is

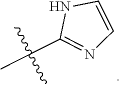 [SMILES: *c1ncc[nH]1]

In other embodiments, R^{3a} is

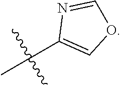 [SMILES: *c1cocn1]

In other embodiments, R^{3a} is

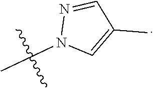 [SMILES: *n1cc(C)cn1]

In other embodiments, R^{3a} is

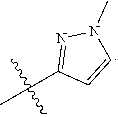 [SMILES: *c1ccn(C)n1]

In other embodiments, R^{3a} is

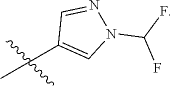 [SMILES: *c1cnn(C(F)F)c1]

In other embodiments, R^{3a} is

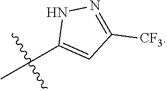 [SMILES: *c1cc(C(F)(F)F)n[nH]1]

In other embodiments, R^{3a} is

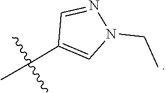 [SMILES: *c1cnn(CC)c1]

In other embodiments, R^{3a} is

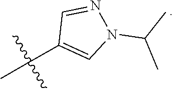 [SMILES: *c1cnn(C(C)C)c1]

In other embodiments, R^{3a} is

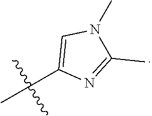 [SMILES: *c1cn(C)c(C)n1 ; "."]

In other embodiments, R^{3a} is

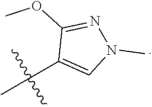

In other embodiments, R^{3a} is

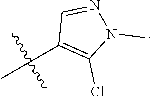 [SMILES: *c1cnn(C)c1Cl]

In other embodiments, R^{3a} is

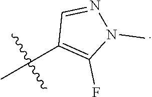 [SMILES: *c1cnn(C)c1F]

In other embodiments, R^{3a} is

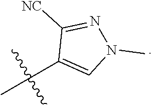 [SMILES: *c1cn(C)nc1C#N]

In other embodiments, R^{3a} is

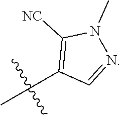 [SMILES: *c1cnn(C)c1C#N]

In other embodiments, R^{3a} is

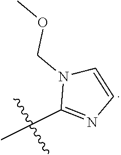 [SMILES: *c1nccn1COC]

In other embodiments, R^{3a} is

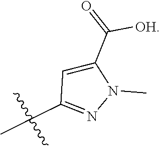 [SMILES: *c1cc(C(=O)O)n(C)n1]

In other embodiments, R^{3a} is

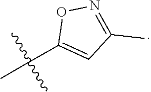 [SMILES: *c1cc(C)no1]

In other embodiments, R^{3a} is

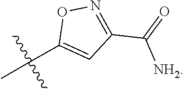 [SMILES: *c1cc(C(N)=O)no1]

In other embodiments, R^{3a} is

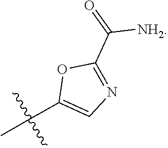 [SMILES: *c1cnc(C(N)=O)o1]

In other embodiments, R^{3a} is

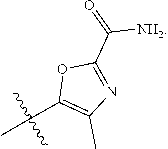 [SMILES: *c1oc(C(N)=O)nc1C]

In other embodiments, R^{3a} is

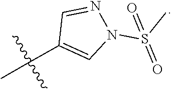 [SMILES: *c1cnn(S(C)(=O)=O)c1]

In other embodiments, R^{3a} is

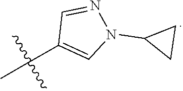 [SMILES: *c1cnn(C2CC2)c1]

In other embodiments, R^{3a} is

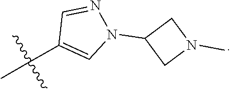 [SMILES: *c1cnn(C2CN(C)C2)c1]

In other embodiments, R^{3a} is

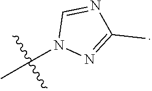 [SMILES: *n1cnc(C)n1]

In other embodiments, R^{3a} is

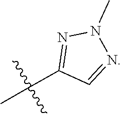 [SMILES: *c1cnn(C)n1]

In other embodiments, R^{3a} is

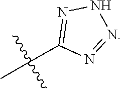 [SMILES: *c1nn[nH]n1]

In other embodiments, R^{3a} is

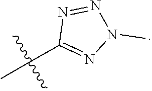 [SMILES: *c1nnn(C)n1]

In other embodiments, R^{3a} is

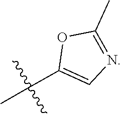 [SMILES: *c1cnc(C)o1]

In other embodiments, R^{3a} is

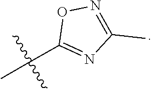 [SMILES: *c1nc(C)no1]

In other embodiments, R^{3a} is

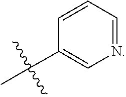 [SMILES: *c1cccnc1]

In other embodiments, R^{3a} is

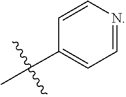 [SMILES: *c1ccncc1]

In other embodiments, R^{3a} is

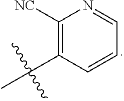 [SMILES: *c1cccnc1C#N]

In other embodiments, R^{3a} is

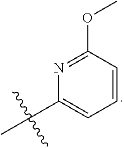 [SMILES: *c1cccc(OC)n1]

In other embodiments, R^{3a} is

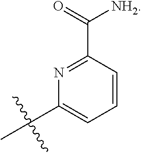 [SMILES: *c1cccc(C(N)=O)n1]

In other embodiments, R^{3a} is

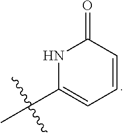 [SMILES: *c1cccc(=O)[nH]1]

In other embodiments, R^{3a} is

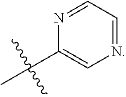 [SMILES: *c1cnccn1]

In other embodiments, R^{3a} is

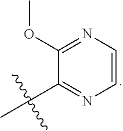 [SMILES: *c1nccnc1OC]

In other embodiments, R^{3a} is

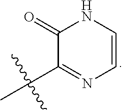 [SMILES: *c1ncc[nH]c1=O]

In other embodiments, R^{3a} is

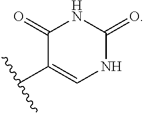 [SMILES: *c1c[nH]c(=O)[nH]c1=O]

In other embodiments, R^{3a} is

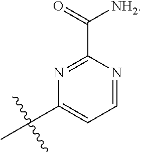 [SMILES: *c1ccnc(C(N)=O)n1]

In other embodiments, R^{3a} is

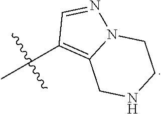 [SMILES: *c1cnn2c1CNCC2]

In other embodiments, R^{3a} is

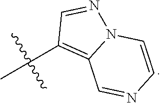 [SMILES: *c1cnn2ccncc12]

In some embodiments, the invention relates to a compound of formula (I-B-1), or a pharmaceutically acceptable salt thereof, wherein R^{5a} is H, halo, C₁-C₆ alkyl, C₁-C₆ alkoxy, —CH₂OH, —CH₂O(C₁-C₆ alkyl), or —C(O)O(C₁-C₆ alkyl), 4-10 membered heteroaryl, wherein said heteroaryl is optionally substituted with R^{a′}; and R^{a′} is —CH₃, —CHF₂, or —CH₂OCH₃. In some embodiments, the invention relates to a compound of formula (I-B-1), or a pharmaceutically acceptable salt thereof, wherein R^{5a} is H, C₁-C₆ alkyl, C₁-C₆ alkoxy, —CH₂OH, —CH₂O(C₁-C₆ alkyl), or —C(O)O(C₁-C₆ alkyl). In other embodiments, R^{5a} is C₁-C₆ alkyl. In other embodiments, R^{5a} is C₁-C₆ alkoxy. In other embodiments, R^{5a} is —CH₂O(C₁-C₆ alkyl). In other embodiments, R^{5a} is —C(O)O(C₁-C₆ alkyl). In other embodiments, R^{5a} is halo. In other embodiments, R^{5a} is 4-10 membered heteroaryl, wherein said heteroaryl is optionally substituted with R^{a′}; and R^{a′} is —CH₃, —CHF₂, or —CH₂OCH₃. In some embodiments, the invention relates to a compound of formula (I-B-1), or a pharmaceutically acceptable salt thereof, wherein R^{5a} is H. Br, —CH₃, —OCH₃, —OCH₂CH₃, —CH₂OH, —CH₂OCH₃, —C(O)OCH₂CH₃,

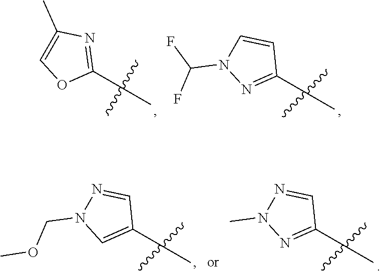

In some embodiments, the invention relates to a compound of formula (I-B-1), or a pharmaceutically acceptable salt thereof, wherein R^{5a} is H, —CH₃, —OCH₃, —OCH₂CH₃, —CH₂OH, —CH₂OCH₃, or —C(O)OCH₂CH₃. In other embodiments, R^{5a} is H. In other embodiments, R^{5a} is —CH₃. In other embodiments, R^{5a} is —OCH₃. In other embodiments, R^{5a} is —OCH₂CH₃. In other embodiments, R^{5a} is —CH₂OH. In other embodiments, R^{5a} is —CH₂OCH₃. In other embodiments, R^{5a} is —C(O)OCH₂CH₃. In other embodiments, R^{5a} is Br. In other embodiments, R^{5a} is

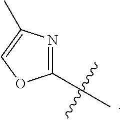 [SMILES: *c1nc(C)co1]

In other embodiments, R^{5a} is

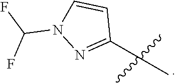 [SMILES: *c1ccn(C(F)F)n1 ; "."]

In other embodiments, R^{5a} is

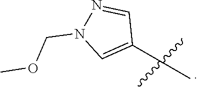 [SMILES: *c1cnn(COC)c1 ; "."]

In other embodiments, R^{5a} is

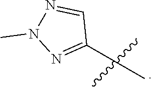 [SMILES: *c1cnn(C)n1 ; "."]

In some embodiments, the invention relates to a compound of formula (I-B-1), or a pharmaceutically acceptable salt thereof, wherein R^{2b} is H, halo, C₁-C₆ alkyl, C₁-C₆ alkoxy, —OH, or —CH₂OH. In other embodiments, R^{2b} is halo. In other embodiments, R^{2b} is C₁-C₆ alkyl. In other embodiments, R^{2b} is C₁-C₆ alkoxy. In some embodiments, the invention relates to a compound of formula (I-B-1), or a pharmaceutically acceptable salt thereof, wherein R^{2b} is H, F, Cl, —CH₃, —CH(CH₃)₂, —C(CH₃)₃, —OCH₃, —OCH₂CH₃, —OH, or —CH₂OH. In other embodiments, R^{2b} is H. In other embodiments, R^{2b} is F. In other embodiments, R^{2b} is Cl. In other embodiments, R^{2b} is —CH₃. In other embodiments, R^{2b} is —CH(CH₃)₂. In other embodiments, R^{2b} is —C(CH₃)₃. In other embodiments, R^{2b} is —OCH₃. In other embodiments, R^{2b} is —OCH₂CH₃. In other embodiments, R^{2b} is —OH. In other embodiments, R^{2b} is —CH₂OH.

In some embodiments, the invention relates to a compound of formula (I-B-1), or a pharmaceutically acceptable salt thereof, wherein R^{3b} is H, halo, C₁-C₆ alkyl, C₁-C₆ haloalkyl, or C₁-C₆ alkoxy. In other embodiments, R^{3b} is halo. In other embodiments, R^{3b} is C₁-C₆ alkyl. In other embodiments, R^{3b} is C₁-C₆ haloalkyl. In other embodiments, R^{3b} is C₁-C₆ alkoxy. In some embodiments, the invention relates to a compound of formula (I-B-1), or a pharmaceutically acceptable salt thereof, wherein R^{3b} is H, F, Cl, —CH₃, —C(CH₃)₃, —CF₃, —OCH₃, —OCH₂CH₃, or —OC(CH₃)₃. In other embodiments, R^{3b} is H. In other embodiments, R^{3b} is F. In other embodiments, R^{3b} is Cl. In other embodiments, R^{3b} is —CH₃. In other embodiments, R^{3b} is —C(CH₃)₃. In other embodiments, R^{3b} is —CF₃. In other embodiments, R^{3b} is —OCH₃. In other embodiments, R^{3b} is —OCH₂CH₃. In other embodiments, R^{3b} is —OC(CH₃)₃.

In some embodiments, the invention relates to a compound of formula (I-B-1), or a pharmaceutically acceptable salt thereof, wherein R^{4b} is H, halo. C₁-C₆ alkyl, C₁-C₆ haloalkyl, C₁-C₆ alkoxy, C₁-C₆ haloalkoxy, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen. In other embodiments, R^{4b} is halo. In other embodiments, R^{4b} is C₁-C₆ alkyl. In other embodiments, R^{4b} is C₁-C₆ haloalkyl. In other embodiments, R^{4b} is C₁-C₆ alkoxy. In other embodiments, R^{4b} is C₁-C₆ haloalkoxy. In other embodiments, R^{4b} is C₃-C₆ cycloalkyl, wherein said cycloalkyl is optionally substituted with one or more halogen. In other embodiments, R^{4b} is (C₁-C₆ alkyl)-(C₃-C₆ cycloalkyl)-, wherein cycloalkyl in said (C₁-C₆ alkyl)-(C₃-C₆ cycloalkyl)- is optionally substituted with one or more halogen. In other embodiments, R^{4b} is (C₁-C₆ haloalkyl)-(C₃-C₆ cycloalkyl)-, wherein cycloalkyl in said (C₁-C₆ haloalkyl)-(C₃-C₆ cycloalkyl)- is optionally substituted with one or more halogen. In some embodiments, the invention relates to a compound of formula (I-B-1), or a pharmaceutically acceptable salt thereof, wherein R^{4b} is H, F, Cl, —CH₃, —CH(CH₃)₂, —C(CH₃)₃, —C(CH₃)₂(CH₂CH₃), —CF₃, —C(CH₃)₂(CF₃)—OCH₃, —OCH(CH₃)₂, —OC(CH₃)₃, —OCF₃, cyclopropyl, 1-trifluoromethylcyclopropyl, 3,3-difluorocyclobutyl, or 3,3-difluoro-1-methylcyclobutyl. In other embodiments, R^{4b} is H. In other embodiments, R^{4b} is F. In other embodiments, R^{4b} is Cl. In other embodiments, R^{4b} is —CH₃. In other embodiments, R^{4b} is —CH(CH₃)₂. In other embodiments, R^{4b} is —C(CH₃)₃. In other embodiments, R^{4b} is —C(CH₃)₂(CH₂CH₃). In other embodiments, R^{4b} is —CF₃. In other embodiments, R^{4b} is —C(CH₃)₂(CF₃). In other embodiments, R^{4b} is —OCH₃. In other embodiments, R^{4b} is —OCH(CH₃)₂. In other embodiments, R^{4b} is —OC(CH₃)₃. In other embodiments, R^{4b} is —OCF₃. In other embodiments, R^{4b} is cyclopropyl. In other embodiments, R^{4b} is 1-trifluoromethylcyclopropyl. In other embodiments, R^{4b} is 3,3-difluorocyclobutyl. In other embodiments, R^{4b} is 3,3-difluoro-1-methylcyclobutyl.

In some embodiments, the invention relates to a compound of formula (I-B-1), or a pharmaceutically acceptable salt thereof, wherein R^{2b} and R^{3b}, together with the carbon atoms to which they are attached, form a ring of formula

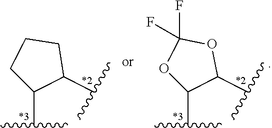 [SMILES: *C1CCCC1* ; "or" ; *C1OC(F)(F)OC1* ; "."]

In some embodiments, the invention relates to a compound of formula (I-B-1), or a pharmaceutically acceptable salt thereof, wherein R^{2b} and R^{3b}, together with the carbon atoms to which they are attached, form a ring of formula

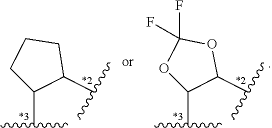 [SMILES: *C1CCCC1* ; "or" ; *C1OC(F)(F)OC1* ; "."]

and R^{4b} is H. In other embodiments, R^{2b} and R^{3b}, together with the carbon atoms to which they are attached, form a ring of formula

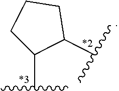 [SMILES: *C1CCCC1* ; "."]

In other embodiments, R^{2b} and R^{3b}, together with the carbon atoms to which they are attached, form a ring of formula

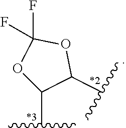 [SMILES: *C1OC(F)(F)OC1* ; "."]

In some embodiments, the invention relates to a compound of formula (I-B-1), or a pharmaceutically acceptable salt thereof, wherein R^{3b} and R^{4b}, together with the carbon atoms to which they are attached, form a ring of formula

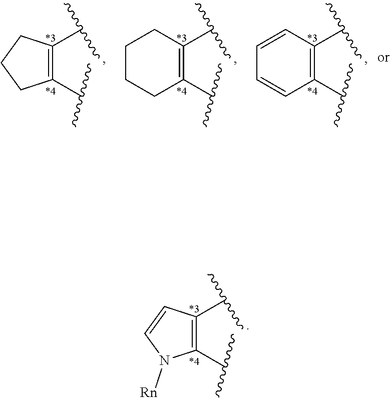

In some embodiments, the invention relates to a compound of formula (I-B-1), or a pharmaceutically acceptable salt thereof, wherein R^{3b} and R^{4b}, together with the carbon atoms to which they are attached, form a ring of formula

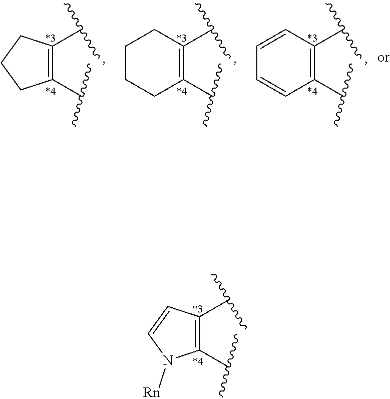

and R^{2b} is H. In other embodiments, R^{3b} and R^{4b}, together with the carbon atoms to which they are attached, form a ring of formula

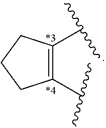 [SMILES: *C1=C(*)CCC1]

In other embodiments, R^{3b} and R^{4b}, together with the carbon atoms to which they are attached, form a ring of formula

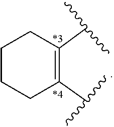 [SMILES: *C1=C(*)CCCC1]

In other embodiments, R^{3b} and R^{4b}, together with the carbon atoms to which they are attached, form a ring of formula

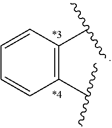 [SMILES: *c1ccccc1*]

In other embodiments, R^{3b} and R^{4b}, together with the carbon atoms to which they are attached, form a ring of formula

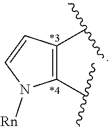

In some embodiments, the invention relates to a compound of formula (I-B-1), or a pharmaceutically acceptable salt thereof, wherein Rₐ is C₁-C₆ alkyl. In some embodiments, the invention relates to a compound of formula (I-B-1), or a pharmaceutically acceptable salt thereof, wherein R^{6b} is —CH₃.

In some embodiments, the invention relates to a compound of formula (I-B-1), or a pharmaceutically acceptable salt thereof, wherein R^{6b} is H, halo, C₁-C₆ alkyl, or C₁-C₆ alkoxy. In other embodiments, R^{6b} is halo. In other embodiments, R^{6b} is C₁-C₆ alkyl. In other embodiments, R^{6b} is C₁-C₆ alkoxy. In some embodiments, the invention relates to a compound of formula (I-B-1), or a pharmaceutically acceptable salt thereof, wherein R^{6b} is H, Cl, —CH₃, or —OCH₃. In other embodiments, R^{6b} is H. In other embodiments, R^{6b} is Cl. In other embodiments, R^{6b} is —CH₃. In other embodiments, R^{6b} is —OCH₃.

In some embodiments, the invention relates to a compound of formula (I-B-2):

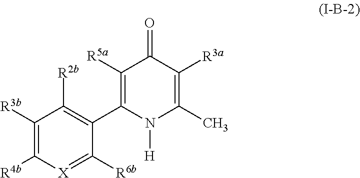

or a pharmaceutically acceptable salt thereof, wherein:
   X is CR^{5b} or N;
   R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂);
   R^{5a} is H, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl);
R^{2b}, R^{3b}, and R^{4b} are defined as follows:
   (i) R^{2b} is H, halo, C₁-C₆ alkyl, C₁-C₆ alkoxy, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
      R^{3b} is H, halo, C₁-C₆ alkyl, C₁-C₆ haloalkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
      R^{4b} is C₂-C₆ alkyl, C₂-C₆ haloalkyl, —OH, —CH₂OH, —C(O)(C₁-C₆ alkyl), —C(O)(C₁-C₆ haloalkyl), C₄-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₄-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; or
   (ii) R^{2b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
   R^{3b} and R^{4b}, together with the carbon atoms to which they are attached, form a ring of formula:

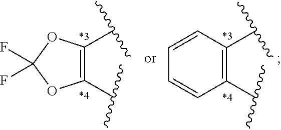 [SMILES: *C1=C(*)OC(F)(F)O1 ; "or" ; *c1ccccc1* ; ";"]

R^{5b} is H, halo, C₁-C₆ alkyl, C₁-C₆ haloalkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
R^{6b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen,
provided that:
   (i) if X is N, then R^{3b} and R^{4b}, together with the carbon atoms to which they are attached, form a ring of formula:

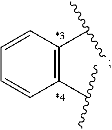 [SMILES: *c1ccccc1*]

and
(ii) no more than one of R^{2b}, R^{3b}, R^{5b}, and R^{6b} is chloro.

In some embodiments, the invention relates to a compound of formula (I-B-2), or a pharmaceutically acceptable salt thereof, wherein X is N. In some embodiments, the invention relates to a compound of formula (I-B-2), or a pharmaceutically acceptable salt thereof, wherein X is CR^{5b}. In some embodiments, the invention relates to a compound of formula (I-B-2), or a pharmaceutically acceptable salt thereof, wherein X is CR^{5b} and R^{5b} is H, halo, C₁-C₆ alkyl, or C₁-C₆ haloalkyl. In other embodiments, R^{5b} is halo. In other embodiments, R^{5b} is C₁-C₆ alkyl. In other embodiments, R^{5b} is C₁-C₆ haloalkyl. In some embodiments, the invention relates to a compound of formula (I-B-2), or a pharmaceutically acceptable salt thereof, wherein R^{5b} is H, F, Cl, —CH₃, or —CF₃. In other embodiments, R^{5b} is H. In other embodiments, R^{5b} is F. In other embodiments, R^{5b} is Cl. In other embodiments, R^{5b} is —CH₃. In other embodiments, R^{5b} is —CF₃.

In some embodiments, the invention relates to a compound of formula (I-B-2), or a pharmaceutically acceptable salt thereof, wherein R^{3a} is H, halo, —CN, C₁-C₆ alkyl, C₁-C₆ alkoxy, —NH₂, —N(C₁-C₆ alkyl)₂, —(C₁-C₆ alkylene)-OH, —(C₁-C₆ alkylene)-NH₂, —C(O)(C₁-C₆ alkyl), —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂). In other embodiments, R^{3a} is halo. In other embodiments, R^{3a} is C₁-C₆ alkyl. In other embodiments, R^{3a} is C₁-C₆ alkoxy. In other embodiments, R^{3a} is —N(C₁-C₆ alkyl)₂. In other embodiments, R^{3a} is —(C₁-C₆ alkylene)-OH. In other embodiments, R^{3a} is —(C₁-C₆ alkylene)-NH₂. In other embodiments, R^{3a} is —C(O)(C₁-C₆ alkyl). In other embodiments, R^{3a} is —C(O)O(C₁-C₆ alkyl). In other embodiments, R^{3a} is —S(O)₂—(C₁-C₆ alkyl). In other embodiments, R^{3a} is —S(O)(NH)(C₁-C₆ alkyl). In other embodiments, R^{3a} is —CH₂C(O)NH(C₁-C₆ alkyl). In other embodiments, R^{3a} is —NHC(O)(C₁-C₆ alkyl). In some embodiments, the invention relates to a compound of formula (I-B-2), or a pharmaceutically acceptable salt thereof, wherein R^{3a} is H, Cl, —CN, —CH₃, —CH(CH₃)₂, —OCH₃, —NH₂, —N(CH₃)₂, —CH₂OH, —CH(CH₃)(OH), —C(CH₃)₂(OH), —CH₂NH₂, —C(O)CH₃, —C(O)OCH₂CH₃, —S(O)₂—(CH₃), —S(O)(NH)(CH₃), —CH₂C(O)NH₂, —CH₂C(O)NH(CH₃), —NHC(O)CH₃, —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂). In other embodiments, R^{3a} is H. In other embodiments, R^{3a} is Cl. In other embodiments, R^{3a} is —CN. In other embodiments, R^{3a} is —CH₃. In other embodiments, R^{3a} is —CH(CH₃)₂. In other embodiments, R^{3a} is —OCH₃. In other embodiments, R^{3a} is —NH₂. In other embodiments, R^{3a} is —N(CH₃)₂. In other embodiments, R^{3a} is —CH₂OH. In other embodiments, R^{3a} is —CH(CH₃)(OH). In other embodiments, R^{3a} is —C(CH₃)₂(OH). In other embodiments, R^{3a} is —CH₂NH₂. In other embodiments, R^{3a} is —C(O)CH₃. In other embodiments, R^{3a} is —C(O)OCH₂CH₃. In other embodiments, R^{3a} is —S(O)₂—(CH₃). In other embodiments, R^{3a} is —S(O)(NH)(CH₃). In other embodiments, R^{3a} is —CH₂C(O)NH₂. In other embodiments, R^{3a} is —CH₂C(O)NH(CH₃). In other embodiments, R^{3a} is —NHC(O)CH₃. In other embodiments, R^{3a} is —CH₂NHC(O)CH(CH₃)(NH₂). In other embodiments, R^{3a} is —CH₂N(CH₃)C(O)CH(CH₃)(N(CH₃)₂). In other embodiments, R^{3a} is —CH₂CH₂NHC(O)CH(CH₃)(NH₂). In other embodiments, R^{3a} is —NHC(O)CH(CH₃)(NH₂).

In some embodiments, the invention relates to a compound of formula (I-B-2), or a pharmaceutically acceptable salt thereof, wherein R^{5a} is H, C₁-C₆ alkyl, C₁-C₆ alkoxy, —CH₂OH, —CH₂O(C₁-C₆ alkyl), or —C(O)O(C₁-C₆ alkyl). In other embodiments, R^{5a} is C₁-C₆ alkyl. In other embodiments, R^{5a} is C₁-C₆ alkoxy. In other embodiments, R^{5a} is —CH₂O(C₁-C₆ alkyl). In other embodiments, R^{5a} is —C(O)O(C₁-C₆ alkyl). In some embodiments, the invention relates to a compound of formula (I-B-2), or a pharmaceutically acceptable salt thereof, wherein R^{5a} is H, —CH₃, —OCH₃, —OCH₂CH₃, —CH₂OH, —CH₂OCH₃, or —C(O)OCH₂CH₃. In other embodiments, R^{5a} is H. In other embodiments, R^{5a} is —CH₃. In other embodiments, R^{5a} is —OCH₃. In other embodiments, R^{5a} is —OCH₂CH₃. In other embodiments, R^{5a} is —CH₂OH. In other embodiments, R^{5a} is —CH₂OCH₃. In other embodiments, R^{5a} is —C(O)OCH₂CH₃.

In some embodiments, the invention relates to a compound of formula (I-B-2), or a pharmaceutically acceptable salt thereof, wherein R^{2b} is H, halo, C₁-C₆ alkyl, or C₁-C₆ alkoxy. In other embodiments, R^{2b} is halo. In other embodiments, R^{2b} is C₁-C₆ alkyl. In other embodiments, RV is C₁-C₆ alkoxy. In some embodiments, the invention relates to a compound of formula (I-B-2), or a pharmaceutically acceptable salt thereof, wherein R^{2b} is H, F, —CH₃, —CH(CH₃)₂, or —OCH₃. In other embodiments, R^{2b} is H. In other embodiments, R^{2b} is F. In other embodiments, R^{2b} is —CH₃. In other embodiments, R^{2b} is —CH(CH₃)₂. In other embodiments, R^{2b} is —OCH₃.

In some embodiments, the invention relates to a compound of formula (I-B-2), or a pharmaceutically acceptable salt thereof, wherein R^{3b} is H or C₁-C₆ alkyl. In other embodiments, R^{3b} is C₁-C₆ alkyl. In some embodiments, the invention relates to a compound of formula (I-B-2), or a pharmaceutically acceptable salt thereof, wherein R^{3b} is H or —CH₃. In other embodiments, R^{3b} is H. In other embodiments, R^{3b} is —CH₃.

In some embodiments, the invention relates to a compound of formula (I-B-2), or a pharmaceutically acceptable salt thereof, wherein R^{4b} is C₂-C₆ alkyl, C₂-C₆ haloalkyl, C₄-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₄-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen. In other embodiments, R^{4b} is C₂-C₆ alkyl. In other embodiments, R^{4b} is C₂-C₆ haloalkyl. In other embodiments, R^{4b} is C₄-C₆ cycloalkyl, wherein said cycloalkyl is optionally substituted with one or more halogen. In other embodiments, R^{4b} is (C₁-C₆ alkyl)-(C₃-C₆ cycloalkyl)-, wherein cycloalkyl in said (C₁-C₆ alkyl)-(C₃-C₆ cycloalkyl)- is optionally substituted with one or more halogen. In other embodiments, R^{4b} is (C₁-C₆ haloalkyl)-(C₃-C₆ cycloalkyl)-, wherein cycloalkyl in said (C₁-C₆ haloalkyl)-(C₃-C₆ cycloalkyl)- is optionally substituted with one or more halogen. In some embodiments, the invention relates to a compound of formula (I-B-2), or a pharmaceutically acceptable salt thereof, wherein R^{4b} is —CH(CH₃)₂, —C(CH₃)₃, —C(CH₃)₂(CH₂CH₃), —C(CH₃)₂(CF₃), 1-trifluoromethylcyclopropyl, 3,3-difluorocyclobutyl, 3,3-difluoro-1-methylcyclobutyl. In other embodiments, R^{4b} is —CH(CH₃)₂. In other embodiments, R^{4b} is —C(CH₃)₃. In other embodiments, R^{4b} is —C(CH₃)₂(CH₂CH₃). In other embodiments, R^{4b} is —C(CH₃)₂(CF₃). In other embodiments, R^{4b} is 1-trifluoromethylcyclopropyl. In other embodiments, R⁴ is 3,3-difluorocyclobutyl. In other embodiments, R^{4b} is 3,3-difluoro-1-methylcyclobutyl.

In some embodiments, the invention relates to a compound of formula (I-B-2), or a pharmaceutically acceptable salt thereof, wherein R^{3b} and R^{4b}, together with the carbon atoms to which they are attached, form a ring of formula

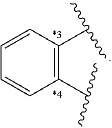 [SMILES: *c1ccccc1*]

In some embodiments, the invention relates to a compound of formula (I-B-2), or a pharmaceutically acceptable salt thereof, wherein R^{3b} and R^{4b}, together with the carbon atoms to which they are attached, form a ring of formula

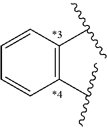 [SMILES: *c1ccccc1*]

### and R^{2b} is H.

In some embodiments, the invention relates to a compound of formula (I-B-2), or a pharmaceutically acceptable salt thereof, wherein R^{6b} is H, C₁-C₆ alkyl, or C₁-C₆ alkoxy. In other embodiments, R^{6b} is C₁-C₆ alkyl. In other embodiments, R is C₁-C₆ alkoxy. In some embodiments, the invention relates to a compound of formula (I-B-2), or a pharmaceutically acceptable salt thereof, wherein R^{6b} is H. —CH₃, or —OCH₃. In other embodiments, R^{6b} is H. In other embodiments, R^{6b} is —CH₃. In other embodiments, R^{6b} is —OCH₃.

In some embodiments, the invention relates to a compound of formula (I-C-1):

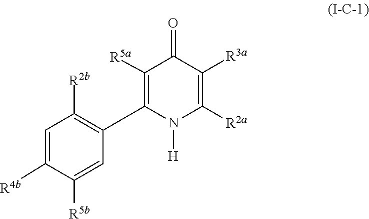

or a pharmaceutically acceptable salt thereof, wherein:
R^{2a} and R^{3a} are defined as follows:
   (i) R^{2a} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂; and
      R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂), —C(O)NHC(O)(C₁-C₆ alkyl), —NHS(O)₂CH₃, —S(C₁-C₆ alkyl), C₃-C₇ cycloalkyl, C₆-C₁₀ aryl, 4-10 membered heterocyclyl, 4-10 membered heteroaryl, wherein said heterocyclyl or heteroaryl is optionally substituted with 1-2 R^{a′}; or
   (ii) R^{3a} is —S(O)R′, —S(O)₂R′, —S(O)(NH)R′, —S(O)(N(C₁-C₆ alkyl))R′, or —S(O)₂NCH₃R′; and
      R^{2a} and R′, together with the atoms to which they are attached, join together to form a 4-7 membered heterocyclyl;
   R^{5a} is H, halo, C₁-C₆ alkyl, C₁-C₆ haloalkyl, C₁-C₆ alkoxy, C₁-C₆ haloalkoxy; —CH₂OH, —CH₂O(C₁-C₆ alkyl), —CH₂O(C₁-C₆ haloalkyl), —C(O)O(C₁-C₆ alkyl), —C(O)O(C₁-C₆ haloalkyl), or 4-10 membered heteroaryl, wherein said heteroaryl is optionally substituted with R^{a′};
   each R^{a′} is independently halo, —CN, —OH, oxo, C₁-C₆ alkyl, C₁-C₆ haloalkyl, C₁-C₆ alkoxy, —(C₁-C₆ alkylene)-(C₁-C₆ alkoxy), —C(O)NH₂, —C(O)OH, —S(O)₂(C₁-C₆ alkyl), C₃-C₇ cycloalkyl, 4-10 membered heterocyclyl, or -(4-10 membered heterocyclyl)-(C₁-C₆ alkyl);
   R^{2b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
   R^{4b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
   R^{5b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen,
provided that:
   (i) if R^{2a} is H, then R^{3a} is halo, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
   (ii) no more than one of R^{2b}, R^{4b}, and R^{5b} is chloro.

In some embodiments, the invention relates to a compound of formula (I-C-1), or a pharmaceutically acceptable salt thereof, wherein:
R^{2a} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
R^{5a} is H, C₁-C₆ alkyl, C₁-C₆ haloalkyl, C₁-C₆ alkoxy, C₁-C₆ haloalkoxy; —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl).

In some embodiments, the invention relates to a compound of formula (I-C-1), or a pharmaceutically acceptable salt thereof, wherein R^{5b} is halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-C-1), or a pharmaceutically acceptable salt thereof, wherein R^{2a} is H or C₁-C₆ alkyl. In other embodiments, R^{2a} is C₁-C₆ alkyl. In some embodiments, the invention relates to a compound of formula (I-C-1), or a pharmaceutically acceptable salt thereof, wherein R^{2a} is H or —CH₃. In other embodiments, R^{2a} is H. In other embodiments, R^{2a} is —CH₃.

In some embodiments, the invention relates to a compound of formula (I-C-1), or a pharmaceutically acceptable salt thereof, wherein R^{3a} is H, halo, —CN, C₁-C₆ alkyl, —C(O)NH₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(CH₃), —CH₂N(CH₃)C(O)CH(CH₃)(N(CH₃)₂), —C(O)NHC(O)CH₃, —NHS(O)₂CH₃, —S(C₁-C₆ alkyl), C₃-C₇ cycloalkyl, C₆-C₁₀ aryl, 4-10 membered heterocyclyl, 4-10 membered heteroaryl, wherein said heterocyclyl or heteroaryl is optionally substituted with 1-2 R^{a′}; and each R^{a′} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₃, cyclopropyl, azetidinyl, or 1-methylazetidinyl. In some embodiments, the invention relates to a compound of formula (I-C-1), or a pharmaceutically acceptable salt thereof, wherein R^{3a} is H, halo, —CN, C₁-C₆ alkyl, —C(O)NH₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(CH₃), or —CH₂N(CH₃)C(O)CH(CH₃)(N(CH₃)₂). In other embodiments, R^{3a} is halo. In other embodiments, R^{3a} is C₁-C₆ alkyl. In other embodiments, R^{3a} is —C(O)O(C₁-C₆ alkyl). In other embodiments, R^{3a} is —S(O)₂—(C₁-C₆ alkyl). In other embodiments, R^{3a} is —S(O)(NH)(C₁-C₆ alkyl). In other embodiments, R^{3a} is —C(O)NHC(O)CH₃. In other embodiments, R^{3a} is —NHS(O)₂CH₃. In other embodiments, R^{3a} is —S(C₁-C₆ alkyl). In other embodiments, R^{3a} is C₃-C₇ cycloalkyl. In other embodiments, R^{3a} is C₆-C₁₀ aryl. In other embodiments, R^{3a} is 4-10 membered heterocyclyl, wherein said heterocyclyl is optionally substituted with 1-2 R^{a′}; and each R^{a′} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₃, cyclopropyl, azetidinyl, or 1-methylazetidinyl. In other embodiments, R^{3a} is 4-10 membered heteroaryl, wherein said heteroaryl is optionally substituted with 1-2 R^{a′}; and each R^{a′} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₃, cyclopropyl, azetidinyl, or 1-methylazetidinyl. In some embodiments, the invention relates to a compound of formula (I-C-1), or a pharmaceutically acceptable salt thereof, wherein R^{3a} is H, Cl, Br, —CN, —CH₃, —C(O)NH₂, —C(O)OCH₂CH₃, —S(O)₂—(CH₃), —S(O)₂CH(CH₃)₂, —S(O)(NH)(CH₃), —S(O)(NH)CH(CH₃)₂, —CH₂C(O)NH₂, —CH₂C(O)NH(CH₃), —CH₂N(CH₃)C(O)CH(CH₃)(N(CH₃)₂), —C(O)NHC(O)CH₃, —NHS(O)₂CH₃, —SCH₃, cyclopropyl, phenyl,

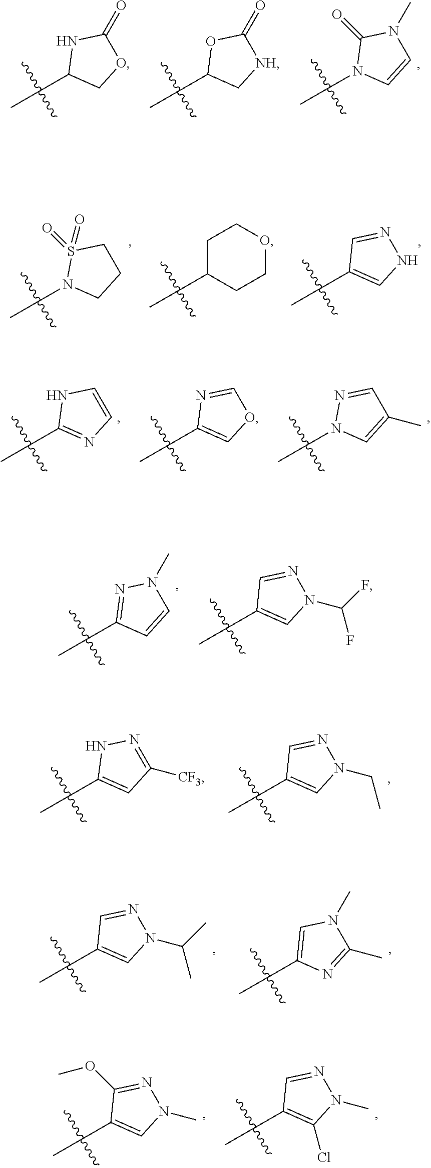

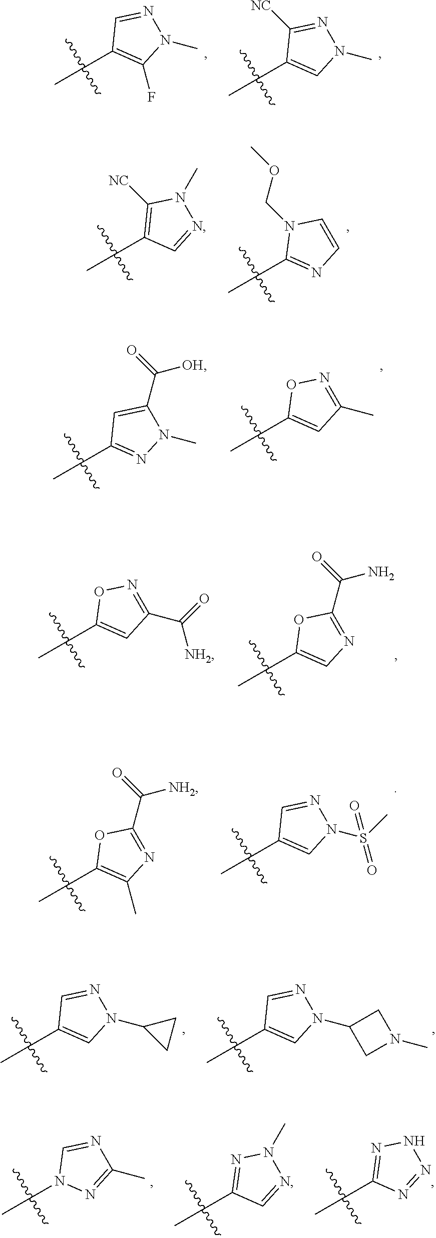

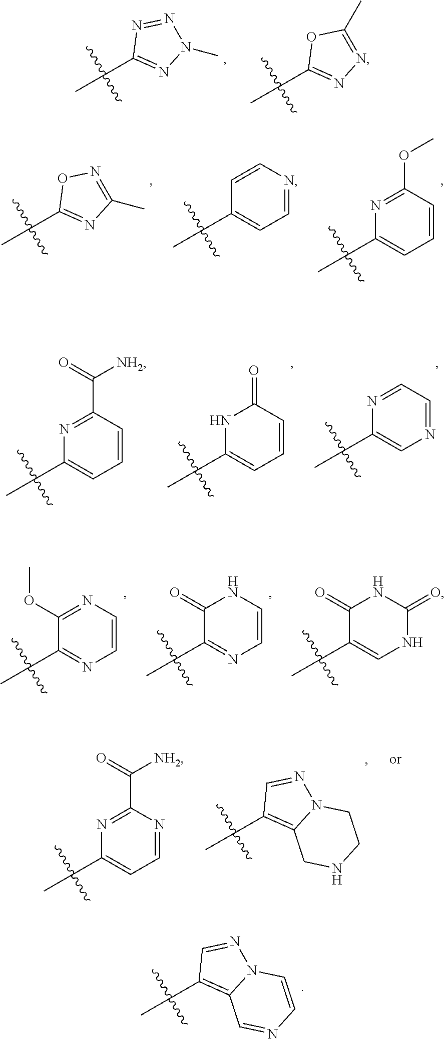

In some embodiments, the invention relates to a compound of formula (I-C-1), or a pharmaceutically acceptable salt thereof, wherein R^{3a} is H, Cl, —CN, —CH₃, —C(O)NH₂, —C(O)OCH₂CH₃, —S(O)₂—(CH₃), —S(O)(NH)(CH₃), —CH₂C(O)NH₂, —CH₂C(O)NH(CH₃), or —CH₂N(CH₃)C(O)CH(CH₃)(N(CH₃)₂). In other embodiments, R^{3a} is H. In other embodiments, R^{3a} is Cl. In other embodiments. R^{3a} is —CN. In other embodiments, R^{3a} is —CH₃. In other embodiments, R^{3a} is —C(O)NH₂. In other embodiments, R^{3a} is —C(O)OCH₂CH₃. In other embodiments, R^{3a} is —S(O)₂—(CH₃). In other embodiments, R^{3a} is —S(O)(NH)(CH₃). In other embodiments, R^{3a} is —CH₂C(O)NH₂. In other embodiments, R^{3a} is —CH₂C(O)NH(CH₃). In other embodiments, R^{3a} is —CH₂N(CH₃)C(O)CH(CH₃)(N(CH₃)₂). In other embodiments, R^{3a} is Br. In other embodiments, R^{3a} is —S(O)₂CH(CH₃)₂. In other embodiments, R^{3a} is —S(O)(NH)CH(CH₃)₂). In other embodiments, R^{3a} is —C(O)NHC(O)CH₃. In other embodiments, R^{3a} is —NHS(O)₂CH₃. In other embodiments, R^{3a} is —SCH₃. In other embodiments. R^{3a} is cyclopropyl. In other embodiments, R^{3a} is phenyl. In other embodiments, R^{3a} is

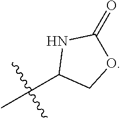 [SMILES: *C1COC(=O)N1]

In other embodiments, R^{3a} is

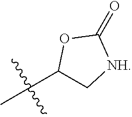 [SMILES: *C1CNC(=O)O1]

In other embodiments, R^{3a} is

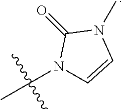 [SMILES: *n1ccn(C)c1=O ; "."]

In other embodiments, R^{3a} is

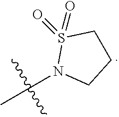 [SMILES: *N1CCCS1(=O)=O ; "."]

In other embodiments, R^{3a} is

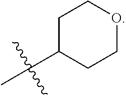 [SMILES: *C1CCOCC1]

In other embodiments, R^{3a} is

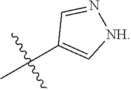 [SMILES: *c1cn[nH]c1]

In other embodiments, R^{3a} is

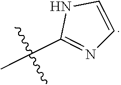 [SMILES: *c1ncc[nH]1]

In other embodiments, R^{3a} is

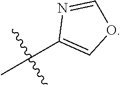 [SMILES: *c1cocn1]

In other embodiments, R^{3a} is

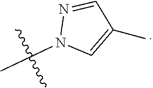 [SMILES: *n1cc(C)cn1 ; "."]

In other embodiments, R^{3a} is

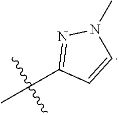 [SMILES: *c1ccn(C)n1]

In other embodiments R^{3a} is

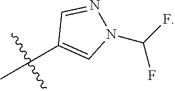 [SMILES: *c1cnn(C(F)F)c1]

In other embodiments, R^{3a} is

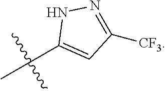 [SMILES: *c1cc(C(F)(F)F)n[nH]1]

In other embodiments, R^{3a} is

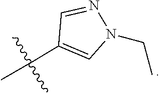 [SMILES: *c1cnn(CC)c1 ; "."]

In other embodiments, R^{3a} is

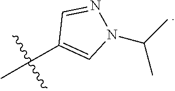 [SMILES: *c1cnn(C(C)C)c1 ; "."]

In other embodiments, R^{3a} is

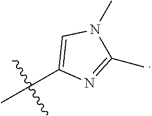 [SMILES: *c1cn(C)c(C)n1 ; "."]

In other embodiments, R^{3a} is

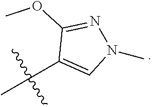

In other embodiments, R^{3a} is

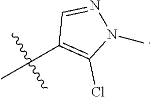 [SMILES: *c1cnn(C)c1Cl ; "."]

In other embodiments, R^{3a} is

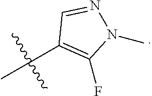 [SMILES: *c1cnn(C)c1F ; "."]

In other embodiments, R^{3a} is

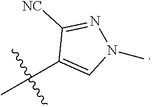 [SMILES: *c1cn(C)nc1C#N ; "."]

In other embodiments. R^{3a} is

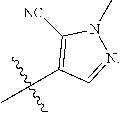 [SMILES: *c1cnn(C)c1C#N]

In other embodiments, R^{3a} is

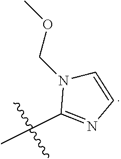 [SMILES: *c1nccn1COC]

In other embodiments, R^{3a} is

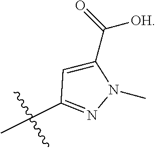 [SMILES: *c1cc(C(=O)O)n(C)n1]

In other embodiments, R^{3a} is

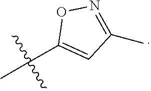 [SMILES: *c1cc(C)no1 ; "."]

In other embodiments, R^{3a} is

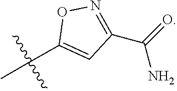 [SMILES: *c1cc(C(N)=O)no1]

In other embodiments, R^{3a} is

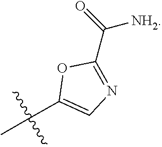 [SMILES: *c1cnc(C(N)=O)o1]

In other embodiments, R^{3a} is

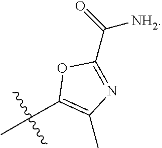 [SMILES: *c1oc(C(N)=O)nc1C]

In other embodiments, R^{3a} is

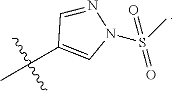 [SMILES: *c1cnn(S(C)(=O)=O)c1 ; "."]

In other embodiments, R^{3a} is

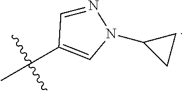 [SMILES: *c1cnn(C2CC2)c1 ; "."]

In other embodiments, R^{3a} is

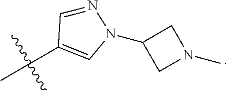 [SMILES: *c1cnn(C2CN(C)C2)c1 ; "."]

In other embodiments, R^{3a} is

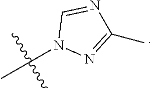 [SMILES: *n1cnc(C)n1 ; "."]

In other embodiments, R^{3a} is

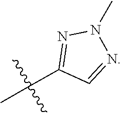 [SMILES: *c1cnn(C)n1]

In other embodiments, R^{3a} is

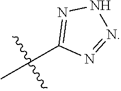 [SMILES: *c1nn[nH]n1]

In other embodiments, R^{3a} is

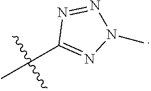 [SMILES: *c1nnn(C)n1 ; "."]

In other embodiments, R^{3a} is

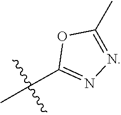 [SMILES: *c1nnc(C)o1]

In other embodiments, R^{3a} is

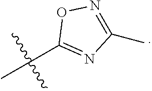 [SMILES: *c1nc(C)no1 ; "."]

In other embodiments, R^{3a} is

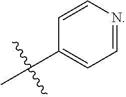 [SMILES: *c1ccncc1]

In other embodiments, R^{3a} is

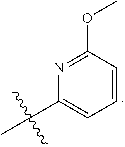 [SMILES: *c1cccc(OC)n1 ; "."]

In other embodiments, R^{3a} is

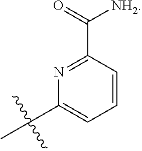 [SMILES: *c1cccc(C(N)=O)n1]

In other embodiments, R^{3a} is

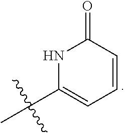 [SMILES: *c1cccc(=O)[nH]1 ; "."]

In other embodiments, R^{3a} is

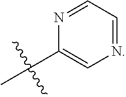 [SMILES: *c1cnccn1]

In other embodiments, R^{3a} is

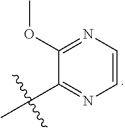 [SMILES: *c1nccnc1OC ; "."]

In other embodiments, R^{3a} is

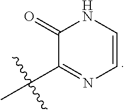 [SMILES: *c1ncc[nH]c1=O ; "."]

In other embodiments, R³ is

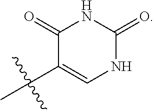 [SMILES: *c1c[nH]c(=O)[nH]c1=O]

In other embodiments, R^{3a} is

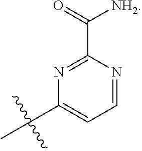 [SMILES: *c1ccnc(C(N)=O)n1]

In other embodiments, R^{3a} is

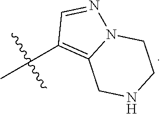 [SMILES: *c1cnn2c1CNCC2]

In other embodiments, R^{3a} is

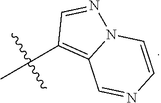 [SMILES: *c1cnn2ccncc12]

In some embodiments, the invention relates to a compound of formula (I-C-1), or a pharmaceutically acceptable salt thereof, wherein R^{3a} is —S(O)R′, —S(O)₂R′, —S(O)(NH)R′, —S(O)(NCH₃)R′, or —S(O)₂NCH₃R′; and R^{2a} and R′, together with the atoms to which they are attached, join together to form a 6-membered heterocyclyl. In other embodiments, R^{3a} is —S(O)R′; and R^{2a} and R′, together with the atoms to which they are attached, join together to form a 6-membered heterocyclyl. In other embodiments, R^{3a} is —S(O)₂R′; and R^{2a} and R′, together with the atoms to which they are attached, join together to form a 6-membered heterocyclyl. In other embodiments, R^{3a} is —S(O)(NH)R′; and R^{2a} and R′, together with the atoms to which they are attached, join together to form a 6-membered heterocyclyl. In other embodiments, R^{3a} is —S(O)(NCH₃)R′. In other embodiments, R^{3a} is —S(O)₂NCH₃R′; and R^{2a} and R′, together with the atoms to which they are attached, join together to form a 6-membered heterocyclyl.

In some embodiments, the invention relates to a compound of formula (I-C-1), or a pharmaceutically acceptable salt thereof, wherein R^{5a} is H, halo, C₁-C₆ alkyl, —C(O)O(C₁-C₆ alkyl), or 4-10 membered heteroaryl, wherein said heteroaryl is optionally substituted with R^{a′}; and R^{a′} is —CH₃, —CHF₂, or —CH₂OCH₃. In some embodiments, the invention relates to a compound of formula (I-C-1), or a pharmaceutically acceptable salt thereof, wherein R^{5a} is H or —C(O)O(C₁-C₆ alkyl). In other embodiments, R^{5a} is —C(O)O(C₁-C₆ alkyl). In other embodiments, R^{5a} is halo. In other embodiments, R^{5a} is C₁-C₆ alkyl. In other embodiments, R^{5a} is 4-10 membered heteroaryl, wherein said heteroaryl is optionally substituted with R^{a′}; and R^{a′} is —CH₃, —CHF₂, or —CH₂OCH₃ In some embodiments, the invention relates to a compound of formula (I-C-1), or a pharmaceutically acceptable salt thereof, wherein H, Br, —CH₃, —C(O)OCH₂CH₃,

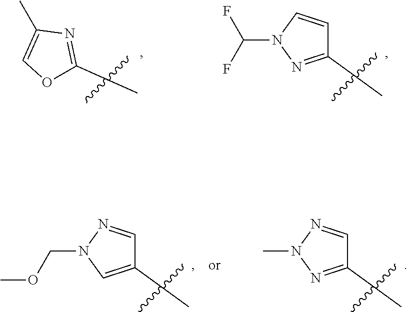

In some embodiments, the invention relates to a compound of formula (I-C-1), or a pharmaceutically acceptable salt thereof, wherein R^{5a} is H or —C(O)OCH₂CH₃. In other embodiments, R^{5a} is H. In other embodiments, R^{5a} is —C(O)OCH₂CH₃. In other embodiments, R^{5a} is Br. In other embodiments, R^{5a} is —CH₃. In other embodiments, R^{5a} is

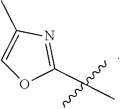 [SMILES: *c1nc(C)co1]

In other embodiments, R^{5a} is

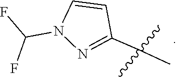 [SMILES: *c1ccn(C(F)F)n1]

In other embodiments, R^{5a} is

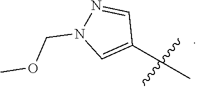 [SMILES: *c1cnn(COC)c1 ; "."]

In other embodiments, R^{5a} is

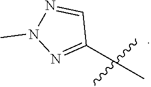 [SMILES: *c1cnn(C)n1 ; "."]

In some embodiments, the invention relates to a compound of formula (I-C-1), or a pharmaceutically acceptable salt thereof, wherein R^{2b} is halo, C₁-C₆ alkyl, or C₁-C₆ alkoxy. In other embodiments, R^{2b} is halo. In other embodiments, R^{2b} is C₁-C₆ alkyl. In other embodiments, R² is C₁-C₆ alkoxy. In some embodiments, the invention relates to a compound of formula (I-C-1), or a pharmaceutically acceptable salt thereof, wherein R^{2b} is F, Cl, —CH₃, or —OCH₃. In other embodiments, R^{2b} is F. In other embodiments, R^{2b} is Cl. In other embodiments, R^{2b} is —CH₃. In other embodiments, R^{2b} is —OCH₃.

In some embodiments, the invention relates to a compound of formula (I-C-1), or a pharmaceutically acceptable salt thereof, wherein R^{4b} is C₁-C₆ alkyl, C₁-C₆ haloalkyl, or C₁-C₆ alkoxy. In other embodiments, R^{4b} is C₁-C₆ alkyl. In other embodiments, R^{4b} is C₁-C₆ haloalkyl. In other embodiments, R^{4b} is C₁-C₆ alkoxy. In some embodiments, the invention relates to a compound of formula (I-C-1), or a pharmaceutically acceptable salt thereof, wherein R^{4b} is —CH₃, —C(CH₃)₃, —C(CH₃)₂(CF₃), or —OCH₃. In other embodiments, R^{4b} is —CH₃. In other embodiments, R^{4b} is —C(CH₃)₃. In other embodiments, R⁴ is —C(CH₃)₂(CF₃). In other embodiments, R^{4b} is —OCH₃.

In some embodiments, the invention relates to a compound of formula (I-C-1), or a pharmaceutically acceptable salt thereof, wherein R^{5b} is halo, C₁-C₆ alkyl, C₁-C₆ haloalkyl, or C₁-C₆ alkoxy. In other embodiments, R^{5b} is halo. In other embodiments, R^{5b} is C₁-C₆ alkyl. In other embodiments, R^{5b} is C₁-C₆ haloalkyl. In other embodiments, R^{5b} is C₁-C₆ alkoxy. In some embodiments, the invention relates to a compound of formula (I-C-1), or a pharmaceutically acceptable salt thereof, wherein R^{5b} is F, Cl, —CH₃, —CF₃, or —OCH₃. In other embodiments, R^{5b} is F. In other embodiments, R^{5b} is Cl. In other embodiments, R^{5b} is —CH₃. In other embodiments, R^{5b} is —CF₃. In other embodiments, R^{5b} is —OCH₃.

In some embodiments, the invention relates to a compound of formula (I-C-1), or a pharmaceutically acceptable salt thereof, wherein:
R^{2b} is halo, C₁-C₆ alkyl, or C₁-C₆ alkoxy;
R^{4b} is C₁-C₆ alkyl, C₁-C₆ haloalkyl, or C₁-C₆ alkoxy; and
R^{5b} is halo, C₁-C₆ alkyl, C₁-C₆ haloalkyl, or C₁-C₆ alkoxy.

In some embodiments, the invention relates to a compound of formula (I-C-2):

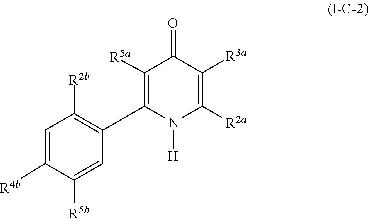

or a pharmaceutically acceptable salt thereof, wherein:
   R^{2a} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
   R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂);
   R^{5a} is H, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl);
   R^{2b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
   R^{4b} is halo, C₂-C₆ alkyl, C₁-C₆ haloalkyl, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
   R^{5b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen,
provided that:
   (i) if R^{2a} is H, then R^{3a} is halo, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
   (ii) no more than one of R^{2b}, R^{4b}, and R^{5b} is chloro.

In some embodiments, the invention relates to a compound of formula (I-C-2), or a pharmaceutically acceptable salt thereof, wherein R^{2b} is halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-C-2), or a pharmaceutically acceptable salt thereof, wherein R^{5b} is halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-C-2), or a pharmaceutically acceptable salt thereof, wherein R^{2a} is H or C₁-C₆ alkyl. In other embodiments, R^{2a} is C₁-C₆ alkyl. In some embodiments, the invention relates to a compound of formula (I-C-2), or a pharmaceutically acceptable salt thereof, wherein R^{2a} is H or —CH₃. In other embodiments, R^{2a} is H. In other embodiments, R^{2a} is —CH₃.

In some embodiments, the invention relates to a compound of formula (I-C-2), or a pharmaceutically acceptable salt thereof, wherein R^{3a} is H, halo, —CN, C₁-C₆ alkyl, —C(O)NH₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), or —CH₂N(CH₃)C(O)CH(CH₃)(N(CH₃)₂). In other embodiments, R^{3a} is halo. In other embodiments, R^{3a} is C₁-C₆ alkyl. In other embodiments, R^{3a} is —C(O)O(C₁-C₆ alkyl). In other embodiments, R^{3a} is —S(O)₂—(C₁-C₆ alkyl). In other embodiments, R^{3a} is —S(O)(NH)(C₁-C₆ alkyl). In some embodiments, the invention relates to a compound of formula (I-C-2), or a pharmaceutically acceptable salt thereof, wherein R^{3a} is H, Cl, —CN, —CH₃, —C(O)NH₂, —C(O)OCH₂CH₃, —S(O)₂—(CH₃), —S(O)(NH)(CH₃), —CH₂C(O)NH₂, —CH₂C(O)NH(CH₃), or —CH₂N(CH₃)C(O)CH(CH₃)(N(CH₃)₂). In other embodiments, R^{3a} is H. In other embodiments, R^{3a} is Cl. In other embodiments, R^{3a} is —CN. In other embodiments, R^{3a} is —CH₃. In other embodiments, R^{3a} is —C(O)NH₂. In other embodiments, R^{3a} is —C(O)OCH₂CH₃. In other embodiments, R^{3a} is —S(O)₂—(CH₃). In other embodiments, R^{3a} is —S(O)(NH)(CH₃). In other embodiments, R^{3a} is —CH₂C(O)NH₂. In other embodiments, R^{3a} is —CH₂C(O)NH(CH₃). In other embodiments, R^{3a} is —CH₂N(CH₃)C(O)CH(CH₃)(N(CH₃)₂).

In some embodiments, the invention relates to a compound of formula (I-C-2), or a pharmaceutically acceptable salt thereof, wherein R^{5a} is H or —C(O)O(C₁-C₆ alkyl). In other embodiments, R^{5a} is —C(O)O(C₁-C₆ alkyl). In some embodiments, the invention relates to a compound of formula (I-C-2), or a pharmaceutically acceptable salt thereof, wherein R^{5a} is H or —C(O)OCH₂CH₃. In other embodiments, R^{5a} is H. In other embodiments, R^{5a} is —C(O)OCH₂CH₃.

In some embodiments, the invention relates to a compound of formula (I-C-2), or a pharmaceutically acceptable salt thereof, wherein R^{2b} is C₁-C₆ alkyl. In some embodiments, the invention relates to a compound of formula (I-C-2), or a pharmaceutically acceptable salt thereof, wherein R^{2b} is —CH₃.

In some embodiments, the invention relates to a compound of formula (I-C-2), or a pharmaceutically acceptable salt thereof, wherein R^{4b} is C₂-C₆ alkyl or C₁-C₆ haloalkyl. In other embodiments, R^{4b} is C₂-C₆ alkyl. In other embodiments, R^{4b} is C₁-C₆ haloalkyl. In some embodiments, the invention relates to a compound of formula (I-C-2), or a pharmaceutically acceptable salt thereof, wherein R^{4b} is —C(CH₃)₃ or —C(CH₃)₂(CF₃). In other embodiments, R^{4b} is —C(CH₃)₃. In other embodiments, R^{4b} is —C(CH₃)₂(CF₃).

In some embodiments, the invention relates to a compound of formula (I-C-2), or a pharmaceutically acceptable salt thereof, wherein R^{5b} is halo, C₁-C₆ alkyl, or C₁-C₆ haloalkyl. In other embodiments, R^{5b} is halo. In other embodiments, R^{5b} is C₁-C₆ alkyl. In other embodiments, R is C₁-C₆ haloalkyl. In some embodiments, the invention relates to a compound of formula (I-C-2), or a pharmaceutically acceptable salt thereof, wherein R^{5b} is F, Cl, —CH₃, or —CF₃. In other embodiments, R⁵ is F. In other embodiments, R^{5b} is Cl. In other embodiments, R^{5b} is —CH₃. In other embodiments, R^{5b} is —CF₃.

In some embodiments, the invention relates to a compound of formula (I-C-2), or a pharmaceutically acceptable salt thereof, wherein:
R^{2b} is C₁-C₆ alkyl;
R^{4b} is C₂-C₆ alkyl or C₁-C₆ haloalkyl; and
R^{5b} is halo, C₁-C₆ alkyl, or C₁-C₆ haloalkyl.

In some embodiments, the invention relates to a compound of formula (I-C-2), or a pharmaceutically acceptable salt thereof, wherein:
R^{2b} is —CH₃;
R^{4b} is —C(CH₃)₃ or —C(CH₃)₂(CF₃); and
R^{5b} is F, Cl, —CH₃, or —CF₃.

In some embodiments, the invention relates to a compound of formula (I-D-1):

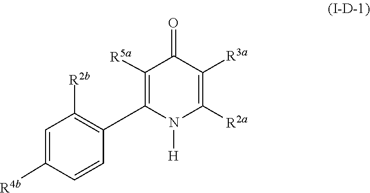

or a pharmaceutically acceptable salt thereof, wherein:
R^{2a} and R^{3a} are defined as follows:
   (i) R^{2a} is C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂; and
      R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂)—C(O)NHC(O)(C₁-C₆ alkyl), —NHS(O)₂CH₃, —S(C₁-C₆ alkyl), C₃-C₇ cycloalkyl, C₆-C₁₀ aryl, 4-10 membered heterocyclyl, 4-10 membered heteroaryl, wherein said heterocyclyl or heteroaryl is optionally substituted with 1-2 R^{a′}; or
   (ii) R^{3a} is —S(O)R′, —S(O)₂R′, —S(O)(NH)R′, —S(O)(N(C₁-C₆ alkyl))R′, or —S(O)₂NCH₃R′; and
      R^{2a} and R′, together with the atoms to which they are attached, join together to form a 4-7 membered heterocyclyl;
   R^{5a} is H, halo, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl), or 4-10 membered heteroaryl, wherein said heteroaryl is optionally substituted with R^{a′};
   each R^{a′} is independently halo, —CN, —OH, oxo, C₁-C₆ alkyl, C₁-C₆ haloalkyl, C₁-C₆ alkoxy, —(C₁-C₆ alkylene)-(C₁-C₆ alkoxy), —C(O)NH₂, —C(O)OH, —S(O)₂(C₁-C₆ alkyl), C₃-C₇ cycloalkyl, 4-10 membered heterocyclyl, or -(4-10 membered heterocyclyl)-(C₁-C₆ alkyl);
   R^{2b} is halo, C₁-C₆ alkyl, C₁-C₆ haloalkyl, C₁-C₆ alkoxy, C₁-C₆ haloalkoxy, —OH. —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
   R^{4b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen,
provided that:
   (i) no more than one of R^{2b} and R^{4b} is chloro; and
   (ii) if R^{4b} is chloro or bromo, then R^{2b} is halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-D-1), or a pharmaceutically acceptable salt thereof, wherein:
R^{2a} is C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₂)(NH₂), or —NHC(O)CH(CH₃)(NH₂); and
R^{5a} is H, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl).

In some embodiments, the invention relates to a compound of formula (I-D-1), or a pharmaceutically acceptable salt thereof, wherein R^{2b} is halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-D-1), or a pharmaceutically acceptable salt thereof, wherein R^{2a} is C₁-C₆ alkyl, C₁-C₆ haloalkyl, C₁-C₆ alkoxy, or —N(C₁-C₆ alkyl)₂. In other embodiments, R^{2a} is C₁-C₆ alkyl. In other embodiments, R^{2a} is C₁-C₆ haloalkyl. In other embodiments, R^{2a} is C₁-C₆ alkoxy. In other embodiments, R^{2a} is —N(C₁-C₆ alkyl)₂. In some embodiments, the invention relates to a compound of formula (I-D-1), or a pharmaceutically acceptable salt thereof, wherein R^{2a} is —CH₃. —CF₃, —OCH₃, or —N(CH₃)₂. In other embodiments, R^{2a} is —CH₃. In other embodiments, R^{2a} is —CF₃. In other embodiments, R^{2a} is —OCH₃. In other embodiments, R^{2a} is —N(CH₃)₂.

In some embodiments, the invention relates to a compound of formula (I-D-1), or a pharmaceutically acceptable salt thereof, wherein R^{3a} is H, halo, C₁-C₆ alkyl, C₁-C₆ alkoxy, —NH₂, —N(C₁-C₆ alkyl)₂, —(C₁-C₆ alkylene)-OH, —(C₁-C₆ alkylene)-NH₂, —C(O)(C₁-C₆ alkyl), —C(O)O(C₁-C₆ alkyl), —S(O)₂—(C₁-C₆ alkyl), —NHC(O)(C₁-C₆ alkyl), —CH₂NHC(O)CH(CH₃)(NH₂), —CH₂CH₂NHC(O)CH(CH₃)(NH₂), —NHC(O)CH(CH₃)(NH₂), —NHS(O)₂CH₃, 4-10 membered heterocyclyl, or 4-10 membered heteroaryl, wherein said heterocyclyl or heteroaryl is optionally substituted with R^{a′}; and R^{a′} is —CN or —CH₃. In some embodiments, the invention relates to a compound of formula (I-D-1), or a pharmaceutically acceptable salt thereof, wherein R^{3a} is H, halo, C₁-C₆ alkyl, C₁-C₆ alkoxy, —NH₂, —N(C₁-C₆ alkyl)₂, —(C₁-C₆ alkylene)-OH, —(C₁-C₆ alkylene)-NH₂, —C(O)(C₁-C₆ alkyl), —C(O)O(C₁-C₆ alkyl), —S(O)₂—(C₁-C₆ alkyl), —NHC(O)(C₁-C₆ alkyl), —CH₂NHC(O)CH(CH₃)(NH₂), —CH₂CH₂NHC(O)CH(CH₃)(NH₂), or —NHC(O)CH(CH₃)(NH₂). In other embodiments, R^{3a} is halo. In other embodiments, R^{3a} is C₁-C₆ alkyl. In other embodiments, R^{3a} is C₁-C₆ alkoxy. In other embodiments, R^{3a} is —N(C₁-C₆ alkyl)₂. In other embodiments, R^{3a} is —(C₁-C₆ alkylene)-OH. In other embodiments, R^{3a} is —(C₁-C₆ alkylene)-NH₂. In other embodiments, R^{3a} is —C(O)(C₁-C₆ alkyl). In other embodiments, R^{3a} is —C(O)O(C₁-C₆ alkyl). In other embodiments, R^{3a} is —S(O)₂—(C₁-C₆ alkyl). In other embodiments, R^{3a} is —NHC(O)(C₁-C₆ alkyl). In other embodiments, R^{3a} is —NHS(O)₂CH₃. In other embodiments, R^{3a} is 4-10 membered heterocyclyl, wherein said heterocyclyl is optionally substituted with R^{a′}; and R^{a′} is —CN or —CH₃. In other embodiments, R^{3a} is 4-10 membered heteroaryl, wherein said heteroaryl is optionally substituted with R^{a′}; and R^{a′} is —CN or —CH₃. In some embodiments, the invention relates to a compound of formula (I-D-1), or a pharmaceutically acceptable salt thereof, wherein R^{3a} is H, Cl, —CH₃, —CH(CH₃)₂, —OCH₃, —NH₂, —N(CH₃)₂, —CH₂OH, —CH(CH₃)(OH), —C(CH₃)₂(OH), —CH₂NH₂, —C(O)CH₃, —C(O)OCH₂CH₃, —S(O)₂—(CH₃), —S(O)₂CH(CH₃)₂, —NHC(O)(CH₃), —CH₂NHC(O)CH(CH₃)(NH₂), —CH₂CH₂NHC(O)CH(CH₃)(NH₂), —NHC(O)CH(CH₃)(NH₂), —NHS(O)₂CH₃,

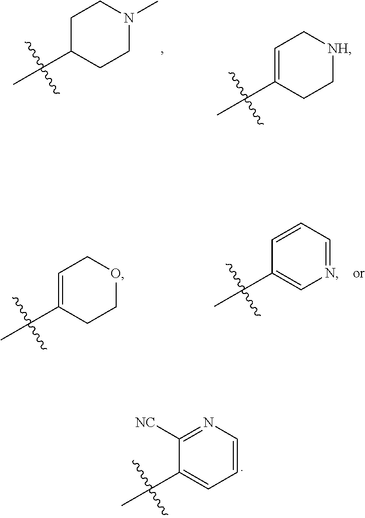

In some embodiments, the invention relates to a compound of formula (I-D-1), or a pharmaceutically acceptable salt thereof, wherein R^{3a} is H, Cl, —CH₃, —CH(CH₃)₂, —OCH₃, —NH₂, —N(CH₃)₂, —CH₂OH, —CH(CH₃)(OH), —C(CH₃)₂(OH), —CH₂NH₂, —C(O)CH₃, —C(O)OCH₂CH₃, —S(O)₂—(CH₃), —NHC(O)(CH₃), —CH₂NHC(O)CH(CH₃)(NH₂), —CH₂CH₂NHC(O)CH(CH₃)(NH₂), or —NHC(O)CH(CH₃)(NH₂). In other embodiments, R^{3a} is H. In other embodiments, R^{3a} is Cl. In other embodiments, R^{3a} is —CH₃. In other embodiments, R^{3a} is —CH(CH₃)₂. In other embodiments, R^{3a} is —OCH₃. In other embodiments, R^{3a} is —NH₂. In other embodiments, R^{3a} is —N(CH₃)₂. In other embodiments, R^{3a} is —CH₂OH. In other embodiments, R^{3a} is —CH(CH₃)(OH). In other embodiments, R^{3a} is —C(CH₃)₂(OH). In other embodiments, R^{3a} is —CH₂NH₂. In other embodiments, R^{3a} is —C(O)CH₃. In other embodiments, R^{3a} is —C(O)OCH₂CH₃. In other embodiments, R^{3a} is —S(O)₂—(CH₃). In other embodiments, R^{3a} is —NHC(O)(CH₃). In other embodiments, R^{3a} is —CH₂NHC(O)CH(CH₃)(NH₂). In other embodiments, R^{3a} is —CH₂CH₂NHC(O)CH(CH₃)(NH₂). In other embodiments, R^{3a} is —NHC(O)CH(CH₃)(NH₂). In other embodiments, R^{3a} is —S(O)₂CH(CH₃)₂. In other embodiments, R^{3a} is —NHS(O)₂CH₃. In other embodiments, R^{3a} is

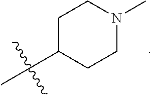 [SMILES: *C1CCN(C)CC1]

In other embodiments, R^{3a} is

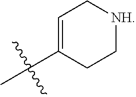 [SMILES: *C1=CCNCC1]

In other embodiments, R^{3a} is

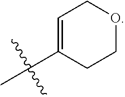 [SMILES: *C1=CCOCC1]

In other embodiments, R^{3a} is

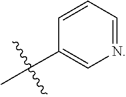 [SMILES: *c1cccnc1]

In other embodiments, R^{3a} is

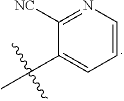 [SMILES: *c1cccnc1C#N]

In some embodiments, the invention relates to a compound of formula (I-D-1), or a pharmaceutically acceptable salt thereof, wherein R^{5a} is H, C₁-C₆ alkyl, C₁-C₆ alkoxy, —CH₂OH, —CH₂O(C₁-C₆ alkyl), or —C(O)O(C₁-C₆ alkyl). In other embodiments, R^{5a} is C₁-C₆ alkyl. In other embodiments, R^{5a} is C₁-C₆ alkoxy. In other embodiments, R^{5a} is —CH₂O(C₁-C₆ alkyl). In other embodiments, R^{5a} is —C(O)O(C₁-C₆ alkyl). In some embodiments, the invention relates to a compound of formula (I-D-1), or a pharmaceutically acceptable salt thereof, wherein R^{5a} is H, —CH₃, —OCH₃, —OCH₂CH₃, —CH₂OH, —CH₂OCH₃, or —C(O)OCH₂CH₃. In other embodiments, R^{5a} is H. In other embodiments, R^{5a} is —CH₃. In other embodiments, R^{5a} is —OCH₃. In other embodiments, R^{5a} is —OCH₂CH₃. In other embodiments, R^{5a} is —CH₂OH. In other embodiments, R^{5a} is —CH₂OCH₃. In other embodiments, R^{5a} is —C(O)OCH₂CH₃.

In some embodiments, the invention relates to a compound of formula (I-D-1), or a pharmaceutically acceptable salt thereof, wherein R^{2b} is halo, C₁-C₆ alkyl, C₁-C₆ alkoxy, or —OH. In other embodiments, R^{2b} is halo. In other embodiments, R^{2b} is C₁-C₆ alkyl. In other embodiments, R^{2b} is C₁-C₆ alkoxy. In some embodiments, the invention relates to a compound of formula (I-D-1), or a pharmaceutically acceptable salt thereof, wherein R^{2b} is F, —CH₃, —CH(CH₃)₂, —OCH₃, —OCH₂CH₃, or —OH. In other embodiments, R^{2b} is F. In other embodiments, R^{2b} is —CH₃. In other embodiments, R^{2b} is —CH(CH₃)₂. In other embodiments, R^{2b} is —OCH₃. In other embodiments, R^{2b} is —OCH₂CH₃. In other embodiments, R^{2b} is —OH.

In some embodiments, the invention relates to a compound of formula (I-D-1), or a pharmaceutically acceptable salt thereof, wherein R^{4b} is halo, C₁-C₆ alkyl, C₁-C₆ haloalkyl, C₁-C₆ alkoxy, C₁-C₆ haloalkoxy, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen. In other embodiments, R^{4b} is halo. In other embodiments, R^{4b} is C₁-C₆ alkyl. In other embodiments, R^{4b} is C₁-C₆ haloalkyl. In other embodiments, R^{4b} is C₁-C₆ alkoxy. In other embodiments, R^{4b} is C₁-C₆ haloalkoxy. In other embodiments, R^{4b} is C₃-C₆ cycloalkyl, wherein said cycloalkyl is optionally substituted with one or more halogen. In other embodiments, R^{4b} is (C₁-C₆ alkyl)-(C₃-C₆ cycloalkyl)-, wherein cycloalkyl in said (C₁-C₆ alkyl)-(C₃-C₆ cycloalkyl)- is optionally substituted with one or more halogen. In other embodiments, R^{4b} is (C₁-C₆ haloalkyl)-(C₃-C₆ cycloalkyl)-, wherein cycloalkyl in said (C₁-C₆ haloalkyl)-(C₃-C₆ cycloalkyl)- is optionally substituted with one or more halogen. In some embodiments, the invention relates to a compound of formula (I-D-1), or a pharmaceutically acceptable salt thereof, wherein R^{4b} is Cl, —CH₃, —CH(CH₃)₂, —C(CH₃)₃, —C(CH₃)₂(CH₂CH₃), —CF₃, —C(CH₃)₂(CF₃), —OCH₃, —OCF₃, cyclopropyl, 1-trifluoromethylcyclopropyl, 3,3-difluorocyclobutyl, or 3,3-difluoro-1-methylcyclobutyl. In other embodiments, R^{4b} is Cl. In other embodiments, R^{4b} is —CH₃. In other embodiments. R⁴ is —CH(CH₃)₂. In other embodiments, R^{4b} is —C(CH₃)₃. In other embodiments, R^{4b} is —C(CH₃)₂(CH₂CH₃). In other embodiments, R^{4U} is —CF. In other embodiments, R^{4b} is —C(CH₃)₂(CF₃). In other embodiments, R^{4b} is —OCH₃. In other embodiments, R^{4b} is —OCF₃. In other embodiments, R^{4b} is cyclopropyl. In other embodiments, R^{4b} is 1-trifluoromethylcyclopropyl. In other embodiments, R^{4b} is 3,3-difluorocyclobutyl. In other embodiments, R^{4b} is 3,3-difluoro-1-methylcyclobutyl.

In some embodiments, the invention relates to a compound of formula (I-D-1), or a pharmaceutically acceptable salt thereof, wherein:
R^{2b} is halo, C₁-C₆ alkyl, C₁-C₆ alkoxy, or —OH; and
R^{4b} is halo, C₁-C₆ alkyl, C₁-C₆ haloalkyl, C₁-C₆ alkoxy, C₁-C₆ haloalkoxy, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-D-2):

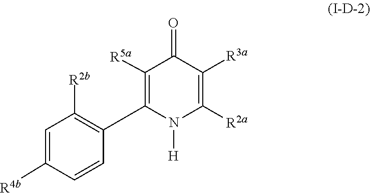

or a pharmaceutically acceptable salt thereof, wherein:
   R^{2a} is C₁-C₆ alkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
   R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂);
   R^{5a} is H, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl);
   R^{2b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
   R^{4b} is C₂-C₆ alkyl, C₂-C₆ haloalkyl, —OH, —CH₂OH, —C(O)(C₁-C₆ alkyl), —C(O)(C₁-C₆ haloalkyl), C₄-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₄-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-D-2), or a pharmaceutically acceptable salt thereof, wherein R^{2b} is halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-D-2), or a pharmaceutically acceptable salt thereof, wherein R^{2a} is C₁-C₆ alkyl, C₁-C₆ alkoxy, or —N(C₁-C₆ alkyl)₂. In other embodiments, R^{2a} is C₁-C₆ alkyl. In other embodiments, R^{2a} is C₁-C₆ alkoxy. In other embodiments, R^{2a} is —N(C₁-C₆ alkyl)₂. In some embodiments, the invention relates to a compound of formula (I-D-2), or a pharmaceutically acceptable salt thereof, wherein R^{2a} is —CH₃, —OCH₃, or —N(CH₃)₂. In other embodiments, R^{2a} is —CH₃. In other embodiments, R^{2a} is —OCH₃. In other embodiments, R^{2a} is —N(CH₃)₂.

In some embodiments, the invention relates to a compound of formula (I-D-2), or a pharmaceutically acceptable salt thereof, wherein R^{3a} is H, halo, C₁-C₆ alkyl, C₁-C₆ alkoxy, —NH₂, —N(C₁-C₆ alkyl)₂, —(C₁-C₆ alkylene)-OH, —(C₁-C₆ alkylene)-NH₂, —C(O)(C₁-C₆ alkyl), —C(O)O(C₁-C₆ alkyl), —S(O)₂—(C₁-C₆ alkyl), —NHC(O)(C₁-C₆ alkyl), —CH₂NHC(O)CH(CH₃)(NH₂), —CH₂CH₂NHC(O)CH(CH₃)(NH₂), or —NHC(O)CH(CH₃)(NH₂). In other embodiments, R^{3a} is halo. In other embodiments, R^{3a} is C₁-C₆ alkyl. In other embodiments, R^{3a} is C₁-C₆ alkoxy. In other embodiments, R^{3a} is —N(C₁-C₆ alkyl)₂. In other embodiments, R^{3a} is —(C₁-C₆ alkylene)-OH. In other embodiments, R^{3a} is —(C₁-C₆ alkylene)-NH₂. In other embodiments, R^{3a} is —C(O)(C₁-C₆ alkyl). In other embodiments, R^{3a} is —C(O)O(C₁-C₆ alkyl). In other embodiments, R^{3a} is —S(O)₂—(C₁-C₆ alkyl). In other embodiments, R^{3a} is —NHC(O)(C₁-C₆ alkyl). In some embodiments, the invention relates to a compound of formula (I-D-2), or a pharmaceutically acceptable salt thereof, wherein R^{3a} is H, Cl, —CH₃, —CH(CH₃)₂, —OCH₃, —NH₂, —N(CH₃)₂, —CH₂OH, —CH(CH₃)(OH), —C(CH₃)₂(OH), —CH₂NH₂, —C(O)CH₃, —C(O)OCH₂CH₃, —S(O)₂—(CH₃), —NHC(O)(CH₃), —CH₂NHC(O)CH(CH₃)(NH₂), —CH₂CH₂NHC(O)CH(CH₃)(NH₂), or —NHC(O)CH(CH₃)(NH₂). In other embodiments, R^{3a} is H. In other embodiments, R^{3a} is Cl. In other embodiments, R^{3a} is —CH₃. In other embodiments, R^{3a} is —CH(CH₃)₂. In other embodiments, R^{3a} is —OCH₃. In other embodiments, R^{3a} is —NH₂. In other embodiments, R^{3a} is —N(CH₃)₂. In other embodiments, R^{3a} is —CH₂OH. In other embodiments, R^{3a} is —CH(CH₃)(OH). In other embodiments, R^{3a} is —C(CH₃)₂(OH). In other embodiments, R^{3a} is —CH₂NH₂. In other embodiments, R^{3a} is —C(O)CH₃. In other embodiments, R^{3a} is —C(O)OCH₂CH₃. In other embodiments, R^{3a} is —S(O)₂—(CH₃). In other embodiments, R^{3a} is —NHC(O)(CH₃). In other embodiments, R^{3a} is —CH₂NHC(O)CH(CH₃)(NH₂). In other embodiments, R^{3a} is —CH₂CH₂NHC(O)CH(CH₃)(NH₂). In other embodiments, R^{3a} is —NHC(O)CH(CH₃)(NH₂).

In some embodiments, the invention relates to a compound of formula (I-D-2), or a pharmaceutically acceptable salt thereof, wherein R^{5a} is H, C₁-C₆ alkyl, C₁-C₆ alkoxy, —CH₂OH, —CH₂O(C₁-C₆ alkyl), or —C(O)O(C₁-C₆ alkyl). In other embodiments, R^{5a} is C₁-C₆ alkyl. In other embodiments, R^{5a} is C₁-C₆ alkoxy. In other embodiments, R^{5a} is —CH₂O(C₁-C₆ alkyl). In other embodiments, R^{5a} is —C(O)O(C₁-C₆ alkyl). In some embodiments, the invention relates to a compound of formula (I-D-2), or a pharmaceutically acceptable salt thereof, wherein R^{5a} is H, —CH₃, —OCH₃, —OCH₂CH₃, —CH₂OH, —CH₂OCH₃, or —C(O)OCH₂CH₃. In other embodiments, R^{5a} is H. In other embodiments, R^{5a} is —CH₃. In other embodiments, R^{5a} is —OCH₃. In other embodiments, R^{5a} is —OCH₂CH₃. In other embodiments, R^{5a} is —CH₂OH. In other embodiments, R^{5a} is —CH₂OCH₃. In other embodiments, R^{5a} is —C(O)OCH₂CH₃.

In some embodiments, the invention relates to a compound of formula (I-D-2), or a pharmaceutically acceptable salt thereof, wherein R^{2b} is C₁-C₆ alkyl or C₁-C₆ alkoxy. In other embodiments, R^{2b} is C₁-C₆ alkyl. In other embodiments, R^{2b} is C₁-C₆ alkoxy. In some embodiments, the invention relates to a compound of formula (I-D-2), or a pharmaceutically acceptable salt thereof, wherein R^{2b} is —CH₃, —CH(CH₃)₂, or —OCH₃. In other embodiments, R^{2b} is —CH₃. In other embodiments, R^{2b} is —CH(CH₃)₂. In other embodiments, R^{2b} is —OCH₃.

In some embodiments, the invention relates to a compound of formula (I-D-2), or a pharmaceutically acceptable salt thereof, wherein R^{4b} is C₂-C₆ alkyl, C₂-C₆ haloalkyl, C₄-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₄-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen. In other embodiments, R^{4b} is C₂-C₆ alkyl. In other embodiments, R^{4b} is C₂-C₆ haloalkyl. In other embodiments, R^{4b} is C₄-C₆ cycloalkyl, wherein said cycloalkyl is optionally substituted with one or more halogen. In other embodiments, R^{4b} is (C₁-C₆ alkyl)-(C₃-C₆ cycloalkyl)-, wherein cycloalkyl in said (C₁-C₆ alkyl)-(C₃-C₆ cycloalkyl)- is optionally substituted with one or more halogen. In other embodiments, R^{4b} is (C₁-C₆ haloalkyl)-(C₃-C₆ cycloalkyl)-, wherein cycloalkyl in said (C₁-C₆ haloalkyl)-(C₃-C₆ cycloalkyl)- is optionally substituted with one or more halogen. In some embodiments, the invention relates to a compound of formula (I-D-2), or a pharmaceutically acceptable salt thereof, wherein R^{4b} is —CH(CH₃)₂, —C(CH₃)₃, —C(CH₃)₂(CH₂CH₃), —C(CH₃)₂(CF₃), 1-trifluoromethylcyclopropyl, 3,3-difluorocyclobutyl or 3,3-difluoro-1-methylcyclobutyl. In other embodiments, R^{4b} is —CH(CH₃)₂. In other embodiments, R^{4b} is —C(CH₃)₃. In other embodiments, R^{4b} is —C(CH₃)₂(CH₂CH₃). In other embodiments, R^{4b} is —C(CH₃)₂(CF₃). In other embodiments, R^{4b} is 1-trifluoromethylcyclopropyl. In other embodiments, R^{4b} is 3,3-difluorocyclobutyl. In other embodiments, R^{4b} is 3,3-difluoro-1-methylcyclobutyl.

In some embodiments, the invention relates to a compound of formula (I-D-2), or a pharmaceutically acceptable salt thereof, wherein:
R^{2b} is C₁-C₆ alkyl or C₁-C₆ alkoxy; and
R^{4b} is C₂-C₆ alkyl, C₂-C₆ haloalkyl, C₄-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₄-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-D-2), or a pharmaceutically acceptable salt thereof, wherein:
R^{2b} is —CH₃, —CH(CH₃)₂, or —OCH₃; and
R^{4b} is —CH(CH₃)₂, —C(CH₃)₃, —C(CH₃)₂(CH₂CH₃), —C(CH₃)₂(CF₃), 1-trifluoromethylcyclopropyl, 3,3-difluorocyclobutyl or 3,3-difluoro-1-methylcyclobutyl.

In some embodiments, the invention relates to a compound of formula (I-E-1):

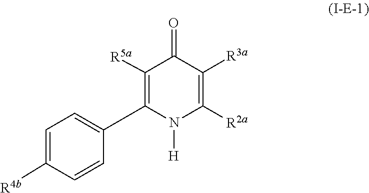

or a pharmaceutically acceptable salt thereof, wherein:
   R^{2a} is C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
   R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂);
   R^{5a} is H, C₁-C₆ alkyl, C₁-C₆ haloalkyl, C₁-C₆ alkoxy, C₁-C₆ haloalkoxy; —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl); and
   R^{4b} is C₁-C₆ alkyl, C₂-C₆ haloalkyl, C₂-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-E-1), or a pharmaceutically acceptable salt thereof, wherein R^{2a} is C₁-C₆ alkyl. In some embodiments, the invention relates to a compound of formula (I-E-1), or a pharmaceutically acceptable salt thereof, wherein R^{2a} is —CH₃.

In some embodiments, the invention relates to a compound of formula (I-E-1), or a pharmaceutically acceptable salt thereof, wherein R^{3a} is H or C₁-C₆ alkyl. In other embodiments, R^{3a} is C₁-C₆ alkyl. In some embodiments, the invention relates to a compound of formula (I-E-1), or a pharmaceutically acceptable salt thereof, wherein R^{3a} is H or —CH₃. In other embodiments, R^{3a} is H. In other embodiments, R^{3a} is —CH₃.

In some embodiments, the invention relates to a compound of formula (I-E-1), or a pharmaceutically acceptable salt thereof, wherein R^{5a} is H or C₁-C₆ alkyl. In other embodiments, R^{5a} is C₁-C₆ alkyl. In some embodiments, the invention relates to a compound of formula (I-E-1), or a pharmaceutically acceptable salt thereof, wherein R^{5a} is H or —CH₃. In other embodiments, R^{5a} is H. In other embodiments, R^{a′} is —CH₃.

In some embodiments, the invention relates to a compound of formula (I-E-1), or a pharmaceutically acceptable salt thereof, wherein R^{4b} is C₁-C₆ alkyl, C₂-C₆ haloalkyl, C₂-C₆ alkoxy, or —(C₁-C₆ haloalkyl)-(C₃-C₆ cycloalkyl), wherein said cycloalkyl is optionally substituted with one or more halogen. In other embodiments, R^{4b} is C₁-C₆ alkyl. In other embodiments, R^{4b} is C₂-C₆ haloalkyl. In other embodiments, R^{4b} is C₂-C₆ alkoxy. In other embodiments, R^{4b} is —(C₁-C₆ haloalkyl)-(C₃-C₆ cycloalkyl), wherein said cycloalkyl is optionally substituted with one or more halogen. In some embodiments, the invention relates to a compound of formula (I-E-1), or a pharmaceutically acceptable salt thereof, wherein R^{4b} is —C(CH₃)₃, —C(CH₃)₂(CF₃), —OC(CH₃)₃, or 1-trifluoromethylcyclopropyl. In other embodiments, R^{4b} is —C(CH₃)₃. In other embodiments, R^{4b} is —C(CH₃)₂(CF₃). In other embodiments, R^{4b} is —OC(CH₃)₃. In other embodiments, R^{4b} is 1-trifluoromethylcyclopropyl.

In some embodiments, the invention relates to a compound of formula (I-E-2):

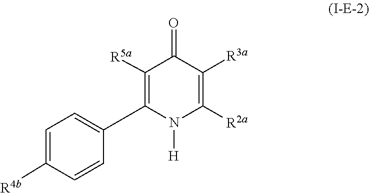

or a pharmaceutically acceptable salt thereof, wherein:
   R^{2a} is C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
   R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂);
   R^{5a} is H, C₁-C₆ alkyl, C₁-C₆ haloalkyl, C₁-C₆ alkoxy, C₁-C₆ haloalkoxy; —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl); and
   R^{4b} is C₁-C₆ alkyl, C₂-C₆ haloalkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-E-2), or a pharmaceutically acceptable salt thereof, wherein R^{2a} is C₁-C₆ alkyl. In some embodiments, the invention relates to a compound of formula (I-E-2), or a pharmaceutically acceptable salt thereof, wherein R^{2a} is —CH₃.

In some embodiments, the invention relates to a compound of formula (I-E-2), or a pharmaceutically acceptable salt thereof, wherein R^{3a} is H or C₁-C₆ alkyl. In other embodiments, R^{3a} is C₁-C₆ alkyl. In some embodiments, the invention relates to a compound of formula (I-E-2), or a pharmaceutically acceptable salt thereof, wherein R^{3a} is H or —CH₃. In other embodiments, R^{3a} is H. In other embodiments, R^{3a} is —CH₃.

In some embodiments, the invention relates to a compound of formula (I-E-2), or a pharmaceutically acceptable salt thereof, wherein R^{5a} is H or C₁-C₆ alkyl. In other embodiments, R^{5a} is C₁-C₆ alkyl. In some embodiments, the invention relates to a compound of formula (I-E-2), or a pharmaceutically acceptable salt thereof, wherein R^{5a} is H or —CH₃. In other embodiments, R^{5a} is H. In other embodiments, R^{5a} is —CH₃.

In some embodiments, the invention relates to a compound of formula (I-E-2), or a pharmaceutically acceptable salt thereof, wherein R^{4b} is C₁-C₆ alkyl, C₂-C₆ haloalkyl, or —(C₁-C₆ haloalkyl)-(C₃-C₆ cycloalkyl), wherein said cycloalkyl is optionally substituted with one or more halogen. In other embodiments, R^{4b} is C₁-C₆ alkyl. In other embodiments, R^{4b} is C₂-C₆ haloalkyl. In other embodiments, R^{4b} is —(C₁-C₆ haloalkyl)-(C₃-C₆ cycloalkyl), wherein said cycloalkyl is optionally substituted with one or more halogen. In some embodiments, the invention relates to a compound of formula (I-E-2), or a pharmaceutically acceptable salt thereof, wherein R^{4b} is —C(CH₃)₃, —C(CH₃)₂(CF₃), or 1-trifluoromethylcyclopropyl. In other embodiments, R^{4b} is —C(CH₃)₃. In other embodiments, R^{4b} is —C(CH₃)₂(CF₃). In other embodiments, R^{4b} is 1-trifluoromethylcyclopropyl.

In some embodiments, the invention relates to a compound of formula (I-F):

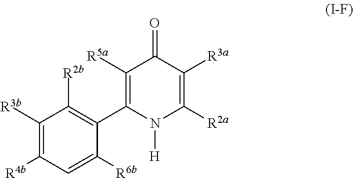

or a pharmaceutically acceptable salt thereof, wherein:
   R^{2a} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
   R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂);
   R^{5a} is H, C₁-C₆ alkyl, C₁-C₆ haloalkyl, C₁-C₆ alkoxy, C₁-C₆ haloalkoxy; —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl);
   R^{2b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
   R^{3b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
   R^{4b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
   R^{6b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-F), or a pharmaceutically acceptable salt thereof, wherein R^{2b} is halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-F), or a pharmaceutically acceptable salt thereof, wherein R^{3b} is halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-F), or a pharmaceutically acceptable salt thereof, wherein R^{6b} is halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

In some embodiments, the invention relates to a compound of formula (I-F), or a pharmaceutically acceptable salt thereof, wherein R^{2a} is C₁-C₆ alkyl. In some embodiments, the invention relates to a compound of formula (I-F), or a pharmaceutically acceptable salt thereof, wherein R^{2a} is —CH₃.

In some embodiments, the invention relates to a compound of formula (I-F), or a pharmaceutically acceptable salt thereof, wherein R^{3a} is —S(O)(NH)(C₁-C₆ alkyl). In some embodiments, the invention relates to a compound of formula (I-F), or a pharmaceutically acceptable salt thereof, wherein R^{3a} is —S(O)(NH)(CH₃).

In some embodiments, the invention relates to a compound of formula (I-F), or a pharmaceutically acceptable salt thereof, wherein R^{5a} is H.

In some embodiments, the invention relates to a compound of formula (I-F), or a pharmaceutically acceptable salt thereof, wherein R^{2b} is halo. In some embodiments, the invention relates to a compound of formula (I-F), or a pharmaceutically acceptable salt thereof, wherein R^{2b} is F.

In some embodiments, the invention relates to a compound of formula (I-F), or a pharmaceutically acceptable salt thereof, wherein R^{3b} is C₁-C₆ alkyl. In some embodiments, the invention relates to a compound of formula (I-F), or a pharmaceutically acceptable salt thereof, wherein R^{3b} is —CH₃.

In some embodiments, the invention relates to a compound of formula (I-F), or a pharmaceutically acceptable salt thereof, wherein R^{4b} is C₁-C₆ alkyl. In some embodiments, the invention relates to a compound of formula (I-F), or a pharmaceutically acceptable salt thereof, wherein R^{4b} is —C(CH₃)₃.

In some embodiments, the invention relates to a compound of formula (I-F), or a pharmaceutically acceptable salt thereof, wherein R^{6b} is C₁-C₆ alkyl. In some embodiments, the invention relates to a compound of formula (I-F), or a pharmaceutically acceptable salt thereof, wherein R^{6b} is —CH₃.

In some embodiments, the invention relates to a compound of formula (I-F), or a pharmaceutically acceptable salt thereof, wherein:
R^{2b} is halo;
R^{3b} is C₁-C₆ alkyl;
R^{4b} is C₁-C₆ alkyl; and
R^{6b} is C₁-C₆ alkyl.

In some embodiments, the invention relates to a compound of formula (I-F), or a pharmaceutically acceptable salt thereof, wherein:
R^{2b} is F;
R^{4b} is —CH₃;
R^{4b} is —C(CH₃)₃; and
R^{6b} is —CH₃.
In some embodiments, the invention relates to a compound of formula (II), or a pharmaceutically acceptable salt thereof, wherein R^{3c} is H, halo, C₁-C₆ alkyl, or —S(O)(NH)(C₁-C₆ alkyl).

In some embodiments, the invention relates to a compound of formula (II), or a pharmaceutically acceptable salt thereof, wherein Y is N. In some embodiments, the invention relates to a compound of formula (II), or a pharmaceutically acceptable salt thereof, wherein Y is CR^{3d}. In some embodiments, the invention relates to a compound of formula (II), or a pharmaceutically acceptable salt thereof, wherein Y is CR^{3d} and R^{3d} is halo, C₁-C₆ alkyl, or C₁-C₆ haloalkyl. In other embodiments, R^{3d} is halo. In other embodiments, R^{3d} is C₁-C₆ alkyl. In other embodiments, R^{3d} is C₁-C₆ haloalkyl. In some embodiments, the invention relates to a compound of formula (II), or a pharmaceutically acceptable salt thereof, wherein Y is CR^{3d} and R^{3d} is Cl, —CH₃, or —CF₃. In other embodiments, R^{3d} is Cl. In other embodiments, R^{3d}—CH₃. In other embodiments, R^{3d}—CF₃.

In some embodiments, the invention relates to a compound of formula (II), or a pharmaceutically acceptable salt thereof, wherein R^{2b} is —CH₃.

In some embodiments, the invention relates to a compound of formula (II), or a pharmaceutically acceptable salt thereof, wherein R^{3c} is H, halo, C₁-C₆ alkyl, —S(O)₂(C₁-C₆ alkyl), —S(O)(NH)(C₁-C₆ alkyl), or 4-10 membered heteroaryl, wherein said heteroaryl is optionally substituted with C₁-C₆ alkyl. In some embodiments, the invention relates to a compound of formula (II), or a pharmaceutically acceptable salt thereof, wherein R^{3c} is H, halo, C₁-C₆ alkyl, or —S(O)(NH)(C₁-C₆ alkyl). In other embodiments. R^{3c} is halo. In other embodiments, R^{3c} is C₁-C₆ alkyl. In other embodiments, R^{3c} is —S(O)(NH)(C₁-C₆ alkyl). In other embodiments, R^{3c} is —S(O)₂(C₁-C₆ alkyl). In other embodiments, R^{3c} is 4-10 membered heteroaryl, wherein said heteroaryl is optionally substituted with C₁-C₆ alkyl. In some embodiments, the invention relates to a compound of formula (II), or a pharmaceutically acceptable salt thereof, wherein R^{3c} is H, Cl, —CH₃, —S(O)₂CH₃, —S(O)(NH)(CH₃), or

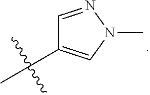 [SMILES: *c1cnn(C)c1]

In some embodiments, the invention relates to a compound of formula (II), or a pharmaceutically acceptable salt thereof, wherein R^{3c} is H, Cl, —CH₃, or —S(O)(NH)(CH₃). In other embodiments, R^{3c} is H. In other embodiments, R^{3c} is Cl. In other embodiments, R^{3c} is —CH₃. In other embodiments, R^{3c} is —S(O)(NH)(CH₃). In other embodiments, R^{3c} is —S(O)₂CH₃. In other embodiments, R^{3c} is

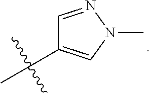 [SMILES: *c1cnn(C)c1]

In some embodiments, the invention relates to a compound of formula (II), or a pharmaceutically acceptable salt thereof, wherein R^{4d} is H, C₁-C₆ alkyl, or C₁-C₆ haloalkyl. In other embodiments, R^{4d} is C₁-C₆ alkyl. In other embodiments, R^{4d} is C₁-C₆ haloalkyl In some embodiments, the invention relates to a compound of formula (II), or a pharmaceutically acceptable salt thereof, wherein R^{4d} is H, —CH₃, —C(CH₃)₃, or —CF₃. In other embodiments, R^{4d} is H. In other embodiments, R^{4d} is —CH₃. In other embodiments, R^{4d} is —C(CH₃)₃. In other embodiments, R^{4d} is or —CF₃.

In some embodiments, the invention relates to a compound of formula (II), or a pharmaceutically acceptable salt thereof, wherein R^{6d} is H, C₁-C₆ alkyl, or C₁-C₆ alkoxy. In other embodiments, R^{6d} is C₁-C₆ alkyl. In other embodiments, R^{6d} is C₁-C₆ alkoxy. In some embodiments, the invention relates to a compound of formula (II), or a pharmaceutically acceptable salt thereof, wherein R^{6d} is H, —CH₃, —OCH₃, —OCH₂CH₃, or —OCH(CH₃)₂. In other embodiments, R^{6d} is H. In other embodiments, R^{6d} is —CH₃. In other embodiments, R^{6d} is —OCH₃. In other embodiments, R^{6d} is —OCH₂CH₃. In other embodiments, R^{6d} is —OCH(CH₃)₂.

In some embodiments, the invention relates to a compound of formula (II-A):

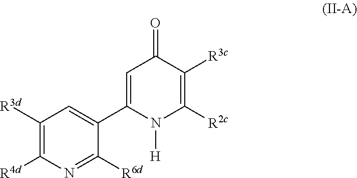

or a pharmaceutically acceptable salt thereof, wherein:
   R^{2c} is C₁-C₆ alkyl;
   R^{3c} is halo, C₁-C₆ alkyl, or —S(O)(NH)(C₁-C₆ alkyl);
   R^{3d} is halo, C₁-C₆ alkyl, or C₁-C₆ haloalkyl;
   R^{4d} is H, C₁-C₆ alkyl, or C₁-C₆ haloalkyl; and
   R^{6d} is H, C₁-C₆ alkyl, or C₁-C₆ alkoxy.

In some embodiments, the invention relates to a compound of formula (II-A), or a pharmaceutically acceptable salt thereof, wherein R^{2c} is —CH₃.

In some embodiments, the invention relates to a compound of formula (II-A), or a pharmaceutically acceptable salt thereof, wherein R^{3c} is halo, C₁-C₆ alkyl, or —S(O)(NH)(C₁-C₆ alkyl). In other embodiments, R^{3c} is halo. In other embodiments, R^{3c} is C₁-C₆ alkyl. In other embodiments, R^{3c} is —S(O)(NH)(C₁-C₆ alkyl). In some embodiments, the invention relates to a compound of formula (II-A), or a pharmaceutically acceptable salt thereof, wherein R^{3c} is Cl, —CH₃, or —S(O)(NH)(CH₃). In other embodiments, R^{3c} is Cl. In other embodiments, R^{3c} is —CH₃. In other embodiments, R^{3c} is —S(O)(NH)(CH₃).

In some embodiments, the invention relates to a compound of formula (IT-A), or a pharmaceutically acceptable salt thereof, wherein R^{3d} is halo or C₁-C₆ alkyl. In other embodiments, R^{3d} is halo. In other embodiments, R^{3d} is C₁-C₆ alkyl. In some embodiments, the invention relates to a compound of formula (II-A), or a pharmaceutically acceptable salt thereof, wherein R^{3d} is Cl or —CH₃. In other embodiments, R^{3d} is Cl. In other embodiments, R^{3d} is —CH₃.

In some embodiments, the invention relates to a compound of formula (II-A), or a pharmaceutically acceptable salt thereof, wherein R^{4d} is C₁-C₆ alkyl. In some embodiments, the invention relates to a compound of formula (II-A), or a pharmaceutically acceptable salt thereof, wherein R^{4d} is —C(CH₃)₃.

In some embodiments, the invention relates to a compound of formula (II-A), or a pharmaceutically acceptable salt thereof, wherein R^{6d} is C₁-C₆ alkyl. In some embodiments, the invention relates to a compound of formula (II-A), or a pharmaceutically acceptable salt thereof, wherein R^{6d} is —CH₃.

In some embodiments, the invention relates to a compound of formula (III), or a pharmaceutically acceptable salt thereof, wherein R^{2e} is —CH₃.

In some embodiments, the invention relates to a compound of formula (III), or a pharmaceutically acceptable salt thereof, wherein R^{2f} is —CH₃.

In some embodiments, the invention relates to a compound of formula (III), or a pharmaceutically acceptable salt thereof, wherein R^{4f} is —C(CH₃)₃.

In some embodiments, the invention relates to a compound of formula (IV), or a pharmaceutically acceptable salt thereof, wherein R^{2g} is C₁-C₆ alkyl. In some embodiments, the invention relates to a compound of formula (IV), or a pharmaceutically acceptable salt thereof, wherein R^{2g} is —CH₃.

In some embodiments, the invention relates to a compound of formula (IV), or a pharmaceutically acceptable salt thereof, wherein R^{3g} is C₁-C₆ alkyl or 4-10 membered heteroaryl, wherein said heteroaryl is optionally substituted with 1 R^{g′}; and R^{g′} is —CH₃. In other embodiments, R^{3g} is C₁-C₆ alkyl. In other embodiments, R^{3g} is 4-10 membered heteroaryl, wherein said heteroaryl is optionally substituted with 1 R^{g′}; and R^{g′} is —CH₃. In some embodiments, the invention relates to a compound of formula (IV), or a pharmaceutically acceptable salt thereof, wherein R^{3g} is —CH₃ or

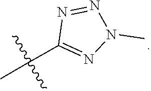 [SMILES: *c1nnn(C)n1]

In other embodiments, R^{3g} is —CH₃. In other embodiments, R^{3g} is

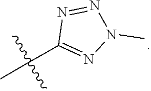 [SMILES: *c1nnn(C)n1]

In some embodiments, the invention relates to a compound of formula (IV), or a pharmaceutically acceptable salt thereof, wherein R^{5g} is H.

In some embodiments, the invention relates to a compound of formula (IV), or a pharmaceutically acceptable salt thereof, wherein R^{2h} is C₁-C₆ alkyl. In some embodiments, the invention relates to a compound of formula (IV), or a pharmaceutically acceptable salt thereof, wherein R^{2h} is —CH₃.

In some embodiments, the invention relates to a compound of formula (IV), or a pharmaceutically acceptable salt thereof, wherein R^{4h} is C₁-C₆ alkyl. In some embodiments, the invention relates to a compound of formula (IV), or a pharmaceutically acceptable salt thereof, wherein R^{4h} is —C(CH₃)₃.

In some embodiments, the invention relates to a compound of formula (IV), or a pharmaceutically acceptable salt thereof, wherein R^{5h} is halo or C₁-C₆ alkyl. In other embodiments, R^{5h} is halo. In other embodiments, R^{5h} is C₁-C₆ alkyl. In some embodiments, the invention relates to a compound of formula (IV), or a pharmaceutically acceptable salt thereof, wherein R^{5h} is Cl or —CH₃. In other embodiments, R^{5h} is Cl. In other embodiments, R^{5h} is —CH₃.

In some embodiments, the invention relates to a compound of formula (IV), or a pharmaceutically acceptable salt thereof, wherein R^{4h} and R^{5h}, together with the C atoms to which they are attached, join together to form a C₅-C₆ cycloalkyl, wherein said cycloalkyl is optionally substituted with 1-2 C₁-C₆ alkyl. In other embodiments, R^{4h} and R^{5h}, together with the C atoms to which they are attached, join together to form a C₆ cycloalkyl, wherein said cycloalkyl is substituted with 2 methyl.

In some embodiments, the invention relates to a compound selected from Table A, or a pharmaceutically acceptable salt thereof. In other embodiments, the invention relates to a compound selected from Table A, i.e., the compound in non-salt form.

TABLE A
Compound Structures and Names. 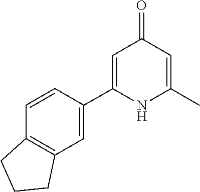 [SMILES: Cc1cc(=O)cc(-c2ccc3c(c2)CCC3)[nH]1]
2-indan-5-yl-6-methyl-1H-pyridin-4-one 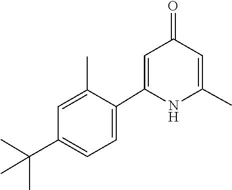 [SMILES: Cc1cc(=O)cc(-c2ccc(C(C)(C)C)cc2C)[nH]1]
2-(4-tert-butyl-2-methyl-phenyl)-6-methyl-1H- pyridin-4-one 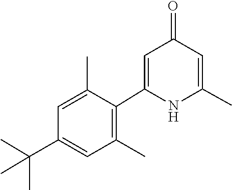 [SMILES: Cc1cc(=O)cc(-c2c(C)cc(C(C)(C)C)cc2C)[nH]1]
2-(4-tert-butyl-2,6-dimethyl-phenyl)-6-methyl-1H- pyridin-4-one 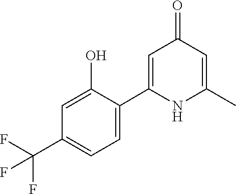 [SMILES: Cc1cc(=O)cc(-c2ccc(C(F)(F)F)cc2O)[nH]1]
2-[2-hydroxy-4-(trifluoromethyl)phenyl]-6-methyl- 1H-pyridin-4-one 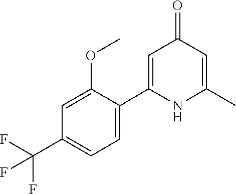 [SMILES: COc1cc(C(F)(F)F)ccc1-c1cc(=O)cc(C)[nH]1]
2-[2-methoxy-4-(trifluoromethyl)phenyl]-6-methyl- 1H-pyridin-4-one 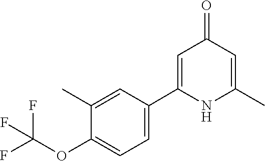 [SMILES: Cc1cc(=O)cc(-c2ccc(OC(F)(F)F)c(C)c2)[nH]1]
2-methyl-6-[3-methyl-4-(trifluoromethoxy)phenyl]- 1H-pyridin-4-one 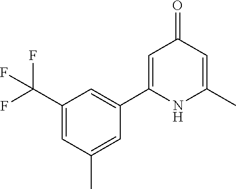 [SMILES: Cc1cc(-c2cc(=O)cc(C)[nH]2)cc(C(F)(F)F)c1]
2-methyl-6-[3-methyl-5-(trifluoromethyl)phenyl]- 1H-pyridin-4-one 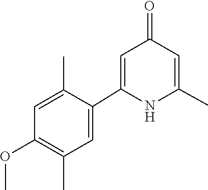 [SMILES: COc1cc(C)c(-c2cc(=O)cc(C)[nH]2)cc1C]
2-(4-methoxy-2,5-dimethyl-phenyl)-6-methyl-1H- pyridin-4-one 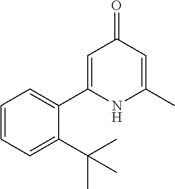 [SMILES: Cc1cc(=O)cc(-c2ccccc2C(C)(C)C)[nH]1]
2-(2-tert-butylphenyl)-6-methyl-1H-pyridin-4-one 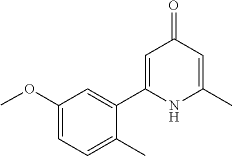
2-(5-methoxy-2-methyl-phenyl)-6-methyl-1H- pyridin-4-one 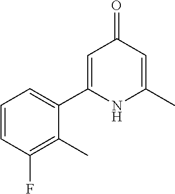 [SMILES: Cc1cc(=O)cc(-c2cccc(F)c2C)[nH]1]
2-(3-fluoro-2-methyl-phenyl)-6-methyl-1H-pyridin- 4-one 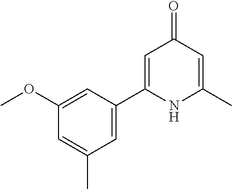
2-(3-methoxy-5-methyl-phenyl)-6-methyl-1H- pyridin-4-one 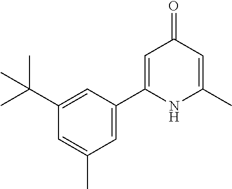 [SMILES: Cc1cc(-c2cc(=O)cc(C)[nH]2)cc(C(C)(C)C)c1]
2-(3-tert-butyl-5-methyl-phenyl)-6-methyl-1H- pyridin-4-one 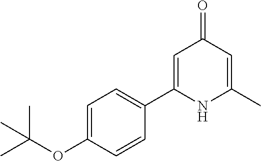 [SMILES: Cc1cc(=O)cc(-c2ccc(OC(C)(C)C)cc2)[nH]1]
2-(4-tert-butoxyphenyl)-6-methyl-1H-pyridin-4-one 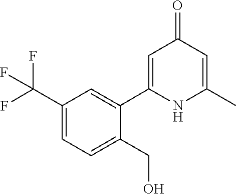 [SMILES: Cc1cc(=O)cc(-c2cc(C(F)(F)F)ccc2CO)[nH]1]
2-[2-(hydroxymethyl)-5-(trifluoromcthyl)phenyl]-6- methyl-1H-pyridin-4-one 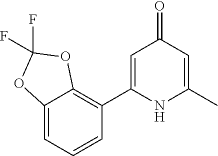 [SMILES: Cc1cc(=O)cc(-c2cccc3c2OC(F)(F)O3)[nH]1]
2-(2,2-difluoro-1,3-benzodioxol-4-yl)-6-methyl-1H- pyridin-4-one 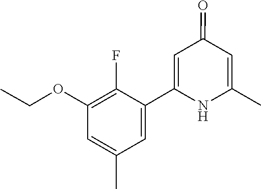 [SMILES: CCOc1cc(C)cc(-c2cc(=O)cc(C)[nH]2)c1F]
2-(3-ethoxy-2-fluoro-5-methyl-phenyl)-6-methyl- 1H-pyridin-4-one 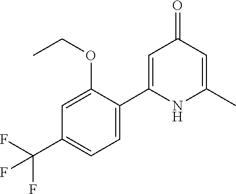 [SMILES: CCOc1cc(C(F)(F)F)ccc1-c1cc(=O)cc(C)[nH]1]
2-[2-ethoxy-4-(trifluoromethyl)phenyl]-6-methyl- 1H-pyridin-4-one 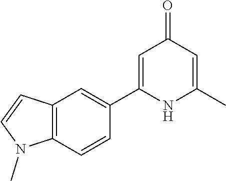 [SMILES: Cc1cc(=O)cc(-c2ccc3c(ccn3C)c2)[nH]1]
2-methyl-6-(1-methylindol-5-yl)-1H-pyridin-4-one 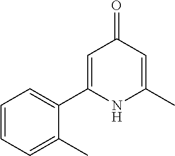
2-methyl-6-(o-tolyl)-1H-pyridin-4-one 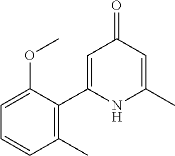 [SMILES: COc1cccc(C)c1-c1cc(=O)cc(C)[nH]1]
2-(2-methoxy-6-methyl-phenyl)-6-methyl-1H- pyridin-4-one 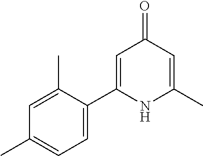
2-(2,4-dimethylphenyl)-6-methyl-1H-pyridin-4-one 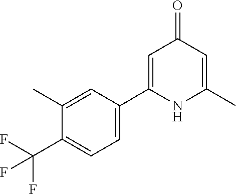 [SMILES: Cc1cc(=O)cc(-c2ccc(C(F)(F)F)c(C)c2)[nH]1]
2-methyl-6-[3-methyl-4-(trifluoromethyl)phenyl]- 1H-pyridin-4-one 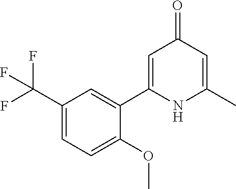 [SMILES: COc1ccc(C(F)(F)F)cc1-c1cc(=O)cc(C)[nH]1]
2-[2-methoxy-5-(trifluoromethyl)phenyl]-6-methyl- 1H-pyridin-4-one 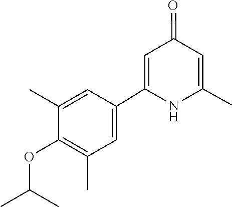 [SMILES: Cc1cc(=O)cc(-c2cc(C)c(OC(C)C)c(C)c2)[nH]1]
2-(4-isopropoxy-3,5-dimethyl-phenyl)-6-methyl-1H- pyridin-4-one 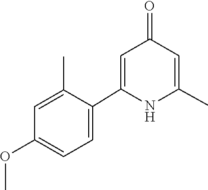 [SMILES: COc1ccc(-c2cc(=O)cc(C)[nH]2)c(C)c1]
2-(4-methoxy-2-methyl-phenyl)-6-methyl-1H- pyridin-4-one 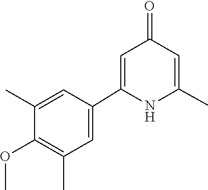 [SMILES: COc1c(C)cc(-c2cc(=O)cc(C)[nH]2)cc1C]
2-(4-methoxy-3,5-dimethyl-phenyl)-6-methyl-1H- pyridin-4-one 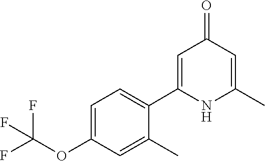 [SMILES: Cc1cc(=O)cc(-c2ccc(OC(F)(F)F)cc2C)[nH]1]
2-methyl-6-[2-methyl-4-(trifluoromethoxy)phenyl]- 1H-pyridin-4-one 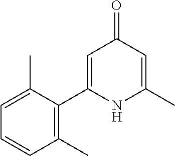 [SMILES: Cc1cc(=O)cc(-c2c(C)cccc2C)[nH]1]
2-(2,6-dimethylphenyl)-6-methyl-1H-pyridin-4-one 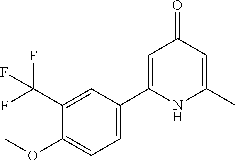 [SMILES: COc1ccc(-c2cc(=O)cc(C)[nH]2)cc1C(F)(F)F]
2-[4-methoxy-3-(trifluoromethyl)phenyl]-6-methyl- 1H-pyridin-4-one 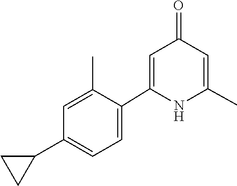 [SMILES: Cc1cc(=O)cc(-c2ccc(C3CC3)cc2C)[nH]1]
2-(4-cyclopropyl-2-methyl-phenyl)-6-methyl-1H- pyridin-4-one 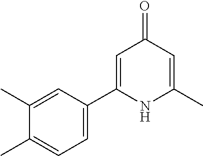
2-(3,4-dimethylphenyl)-6-methyl-1H-pyridin-4-one 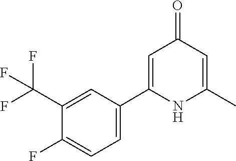 [SMILES: Cc1cc(=O)cc(-c2ccc(F)c(C(F)(F)F)c2)[nH]1]
2-[4-fluoro-3-(trifluoromethyl)phenyl]-6-methyl- 1H-pyridin-4-one 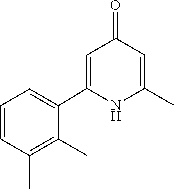 [SMILES: Cc1cc(=O)cc(-c2cccc(C)c2C)[nH]1]
2-(2,3-dimethylphenyl)-6-methyl-1H-pyridin-4-one 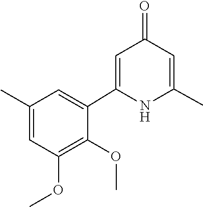
2-(2,3-dimethoxy-5-methyl-phenyl)-6-methyl-1H- pyridin-4-one 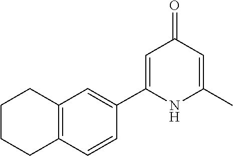 [SMILES: Cc1cc(=O)cc(-c2ccc3c(c2)CCCC3)[nH]1]
2-methyl-6-tetralin-6-yl-1H-pyridin-4-onc 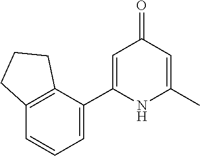 [SMILES: Cc1cc(=O)cc(-c2cccc3c2CCC3)[nH]1]
2-indan-4-yl-6-methyl-1H-pyridin-4-one 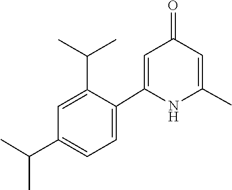 [SMILES: Cc1cc(=O)cc(-c2ccc(C(C)C)cc2C(C)C)[nH]1]
2-(2,4-diisopropylphenyl)-6-methyl-1H-pyridin-4- one 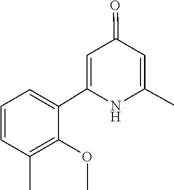 [SMILES: COc1c(C)cccc1-c1cc(=O)cc(C)[nH]1]
2-(2-methoxy-3-methyl-phenyl)-6-methyl-1H- pyridin-4-one 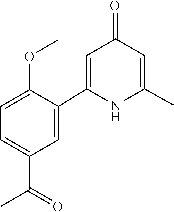 [SMILES: COc1ccc(C(C)=O)cc1-c1cc(=O)cc(C)[nH]1]
2-(5-acetyl-2-methoxy-phenyl)-6-methyl-1H- pyridin-4-one 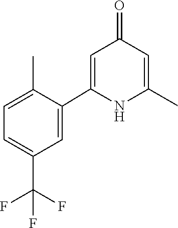 [SMILES: Cc1cc(=O)cc(-c2cc(C(F)(F)F)ccc2C)[nH]1]
2-methyl-6-[2-methyl-5-(trifluoromethyl)phenyl]- 1H-pyridin-4-one 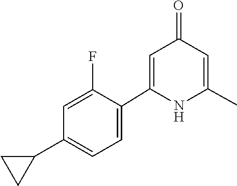 [SMILES: Cc1cc(=O)cc(-c2ccc(C3CC3)cc2F)[nH]1]
2-(4-cyclopropyl-2-fluoro-phenyl)-6-methyl-1H- pyridin-4-one 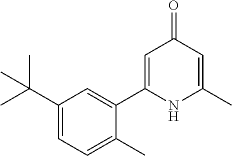 [SMILES: Cc1cc(=O)cc(-c2cc(C(C)(C)C)ccc2C)[nH]1]
2-(5-tert-butyl-2-methyl-phenyl)-6-methyl-1H- pyridin-4-one 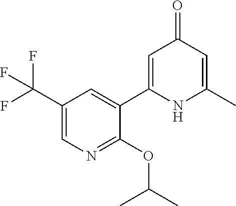 [SMILES: Cc1cc(=O)cc(-c2cc(C(F)(F)F)cnc2OC(C)C)[nH]1]
2-[2-isopropoxy-5-(trifluoromethyl)-3-pyridyl]-6- methyl-1H-pyridin-4-one 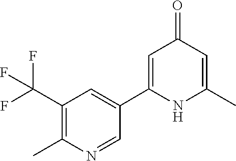 [SMILES: Cc1cc(=O)cc(-c2cnc(C)c(C(F)(F)F)c2)[nH]1]
2-methyl-6-[6-methyl-5-(trifluoromethyl)-3- pyridyl]-1H-pyridin-4-one 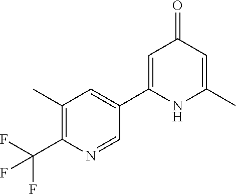 [SMILES: Cc1cc(=O)cc(-c2cnc(C(F)(F)F)c(C)c2)[nH]1]
2-methyl-6-[5-methyl-6-(trifluoromethyl)-3- pyridyl]-1H-pyridin-4-one 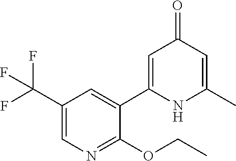 [SMILES: CCOc1ncc(C(F)(F)F)cc1-c1cc(=O)cc(C)[nH]1]
2-[2-ethoxy-5-(trifluoromethyl)-3-pyridyl]-6- methyl-1H-pyridin-4-one 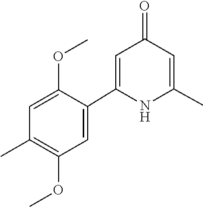
2-(2,5-dimethoxy-4-methyl-phenyl)-6-methyl-1H- pyridin-4-one 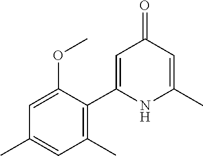
2-(2-methoxy-4,6-dimethyl-phenyl)-6-methyl-1H- pyridin-4-one 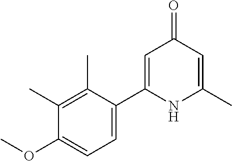
2-(4-methoxy-2,3-dimethyl-phenyl)-6-methyl-1H- pyridin-4-one 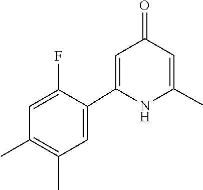
2-(2-fluoro-4,5-dimethyl-phenyl)-6-methyl-1H- pyridin-4-one 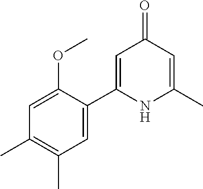
2-(2-methoxy-4,5-dimethyl-phenyl)-6-methyl-1H- pyridin-4-one 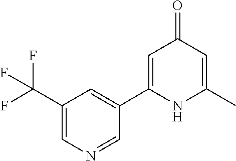 [SMILES: Cc1cc(=O)cc(-c2cncc(C(F)(F)F)c2)[nH]1]
2-methyl-6-[5-(trifluoromethyl)-3-pyridyl]-1H- pyridin-4-one 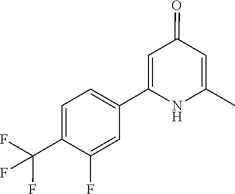 [SMILES: Cc1cc(=O)cc(-c2ccc(C(F)(F)F)c(F)c2)[nH]1]
2-[3-fluoro-4-(trifluoromethyl)phenyl]-6-methyl- 1H-pyridin-4-one 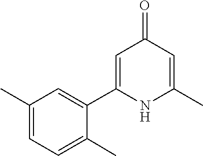
2-(2,5-dimethylphenyl)-6-methyl-1H-pyridin-4-one 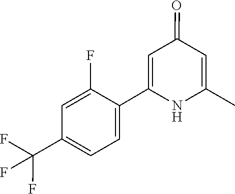 [SMILES: Cc1cc(=O)cc(-c2ccc(C(F)(F)F)cc2F)[nH]1]
2-[2-fluoro-4-(trifluoromcthyl)phenyl]-6-methyl- 1H-pyridin-4-one 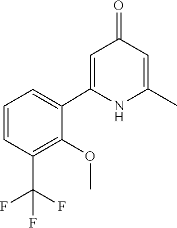 [SMILES: COc1c(-c2cc(=O)cc(C)[nH]2)cccc1C(F)(F)F]
2-[2-methoxy-3-(trifluoromethyl)phenyl]-6-methyl- 1H-pyridin-4-one 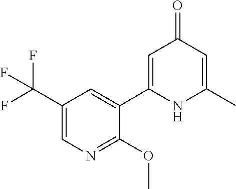 [SMILES: COc1ncc(C(F)(F)F)cc1-c1cc(=O)cc(C)[nH]1]
2-[2-methoxy-5-(trifluoromethyl)-3-pyridyl]-6- methyl-1H-pyridin-4-one 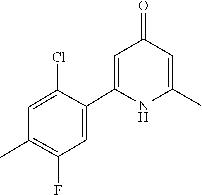
2-(2-chloro-5-fluoro-4-methyl-phenyl)-6-methyl- 1H-pyridin-4-one 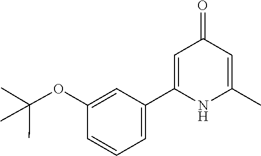 [SMILES: Cc1cc(=O)cc(-c2cccc(OC(C)(C)C)c2)[nH]1]
2-(3-tert-butoxyphenyl)-6-methyl-1H-pyridin-4-one 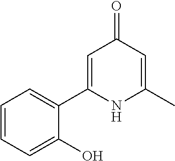
2-(2-hydroxyphenyl)-6-methyl-1H-pyridin-4-one 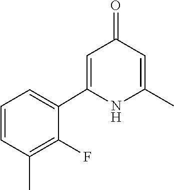 [SMILES: Cc1cc(=O)cc(-c2cccc(C)c2F)[nH]1]
2-(2-fluoro-3-methyl-phenyl)-6-methyl-1H-pyridin- 4-one 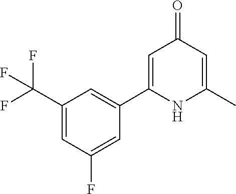 [SMILES: Cc1cc(=O)cc(-c2cc(F)cc(C(F)(F)F)c2)[nH]1]
2-[3-fluoro-5-(trifluoromethyl)phenyl]-6-methyl- 1H-pyridin-4-one 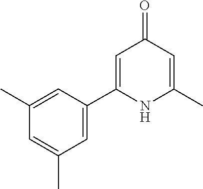
2-(3,5-dimethylphenyl)-6-methyl-1H-pyridin-4-one 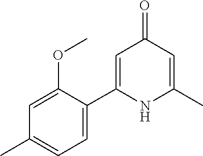
2-(2-methoxy-4-methyl-phenyl)-6-methyl-1H- pyridin-4-one 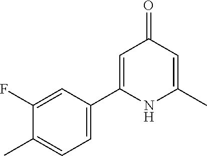
2-(3-fluoro-4-methyl-phenyl)-6-methyl-1H-pyridin- 4-one 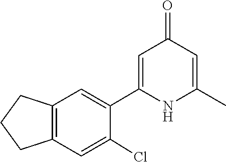 [SMILES: Cc1cc(=O)cc(-c2cc3c(cc2Cl)CCC3)[nH]1]
2-(6-chloroindan-5-yl)-6-methyl-1H-pyridin-4-one 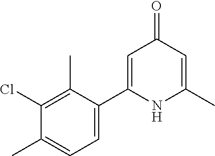
2-(3-chloro-2,4-dimethyl-phenyl)-6-methyl-1H- pyridin-4-one 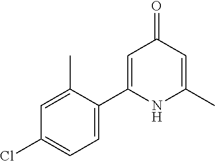 [SMILES: Cc1cc(=O)cc(-c2ccc(Cl)cc2C)[nH]1]
2-(4-chloro-2-methyl-phenyl)-6-methyl-1H-pyridin- 4-one 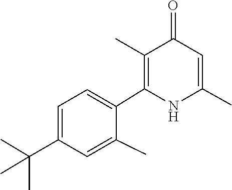 [SMILES: Cc1cc(=O)c(C)c(-c2ccc(C(C)(C)C)cc2C)[nH]1]
2-(4-tert-butyl-2-methyl-phenyl)-3,6-dimethyl-1H- pyridin-4-one 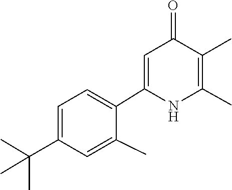 [SMILES: Cc1cc(C(C)(C)C)ccc1-c1cc(=O)c(C)c(C)[nH]1]
6-(4-tert-butyl-2-methyl-phenyl)-2,3-dimethyl-1H- pyridin-4-one 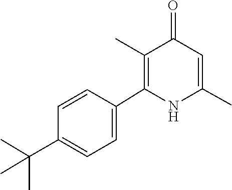 [SMILES: Cc1cc(=O)c(C)c(-c2ccc(C(C)(C)C)cc2)[nH]1]
2-(4-tert-butylphenyl)-3,6-dimethyl-1H-pyridin-4- one 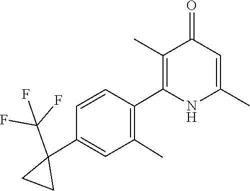 [SMILES: Cc1cc(=O)c(C)c(-c2ccc(C3(C(F)(F)F)CC3)cc2C)[nH]1]
3,6-dimethyl-2-[4-[1- (trifluoromethyl)cyclopropyl]phenyl]-1H-pyridin-4- one 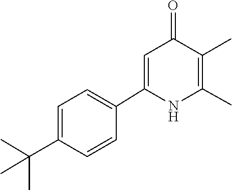 [SMILES: Cc1[nH]c(-c2ccc(C(C)(C)C)cc2)cc(=O)c1C]
6-(4-tert-butylphenyl)-2,3-dimethyl-1H-pyridin-4- one 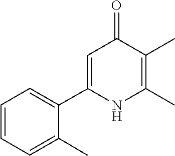 [SMILES: Cc1ccccc1-c1cc(=O)c(C)c(C)[nH]1]
2,3-dimethyl-6-(o-tolyl)-1H-pyridin-4-one 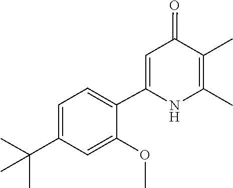 [SMILES: COc1cc(C(C)(C)C)ccc1-c1cc(=O)c(C)c(C)[nH]1]
6-(4-tert-butyl-2-methoxy-phenyl)-2,3-dimethyl-1H- pyridin-4-one 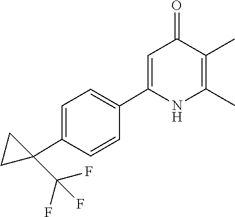 [SMILES: Cc1[nH]c(-c2ccc(C3(C(F)(F)F)CC3)cc2)cc(=O)c1C]
2,3-dimethyl-6-[4-[1- (trifluoromethyl)cyclopropyl]phenyl]-1H-pyridin-4- one 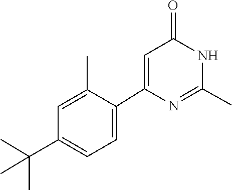 [SMILES: Cc1nc(-c2ccc(C(C)(C)C)cc2C)cc(=O)[nH]1]
4-(4-tert-butyl-2-methyl-phenyl)-2-methyl-1H- pyrimidin-6-one 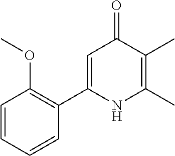 [SMILES: COc1ccccc1-c1cc(=O)c(C)c(C)[nH]1]
6-(2-methoxyphenyl)-2,3-dimethyl-1H-pyridin-4- one 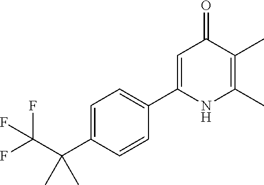 [SMILES: Cc1[nH]c(-c2ccc(C(C)(C)C(F)(F)F)cc2)cc(=O)c1C]
2,3-dimethyl-6-[4-(2,2,2-trifluoro-1,1-dimethyl- ethyl)phenyl]-1H-pyridin-4-one 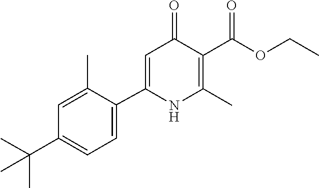 [SMILES: CCOC(=O)c1c(C)[nH]c(-c2ccc(C(C)(C)C)cc2C)cc1=O]
ethyl 6-(4-tert-butyl-2-methyl-phenyl)-2-methyl-4- oxo-1H-pyridine-3-carboxylate 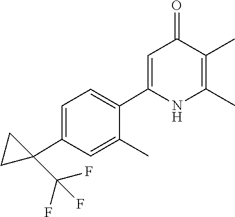 [SMILES: Cc1cc(C2(C(F)(F)F)CC2)ccc1-c1cc(=O)c(C)c(C)[nH]1]
2,3-dimethyl-6-[2-methyl-4-[1- (trifluoromcthyl)cyclopropyl]phenyl]-1H-pyridin-4- one 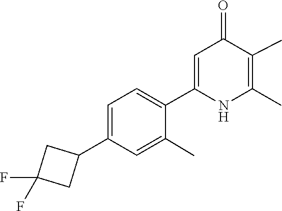 [SMILES: Cc1cc(C2CC(F)(F)C2)ccc1-c1cc(=O)c(C)c(C)[nH]1]
6-[4-(3,3-difluorocyclobutyl)-2-methyl-phenyl]-2,3- dimethyl-1H-pyridin-4-one 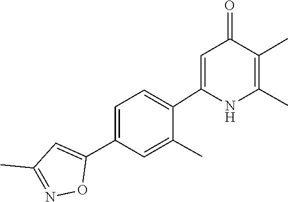
2,3-dimethyl-6-[2-methyl-4-(3-methylisoxazol-5- yl)phenyl]-1H-pyridin-4-one 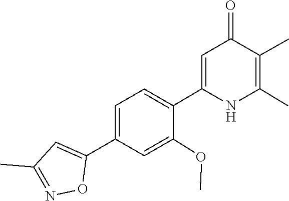
6-[2-methoxy-4-(3-methylisoxazol-5-yl)phenyl]-2,3- dimethyl-1H-pyridin-4-one 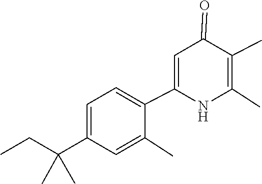 [SMILES: CCC(C)(C)c1ccc(-c2cc(=O)c(C)c(C)[nH]2)c(C)c1]
6-[4-(1,1-dimethylpropyl)-2-methyl-phenyl]-2,3- dimethyl-1H-pyridin-4-one 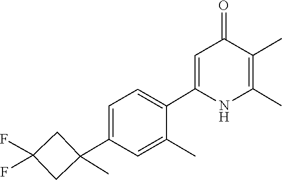 [SMILES: Cc1cc(C2(C)CC(F)(F)C2)ccc1-c1cc(=O)c(C)c(C)[nH]1]
6-[4-(3,3-difluoro-1-methyl-cyclobutyl)-2-methyl- phenyl]-2,3-dimethyl-1H-pyridin-4-one 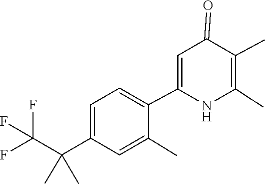 [SMILES: Cc1cc(C(C)(C)C(F)(F)F)ccc1-c1cc(=O)c(C)c(C)[nH]1]
2,3-dimethyl-6-[2-methyl-4-(2,2,2-trifluoro-1,1- dimethyl-ethyl)phenyl]-1H-pyridin-4-one 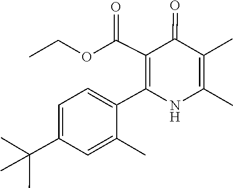 [SMILES: CCOC(=O)c1c(-c2ccc(C(C)(C)C)cc2C)[nH]c(C)c(C)c1=O]
ethyl 2-(4-tert-butyl-2-methyl-phenyl)-5,6-dimethyl- 4-oxo-1H-pyridine-3-carboxylate 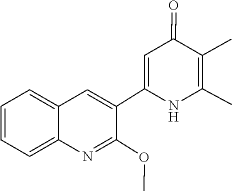 [SMILES: COc1nc2ccccc2cc1-c1cc(=O)c(C)c(C)[nH]1]
6-(2-methoxy-3-quinolyl)-2,3-dimethyl-1H-pyridin- 4-one 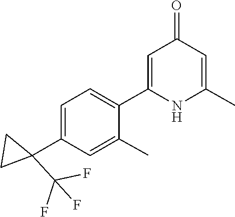 [SMILES: Cc1cc(=O)cc(-c2ccc(C3(C(F)(F)F)CC3)cc2C)[nH]1]
2-methyl-6-[2-methyl-4-[1- (trifluoromethyl)cyclopropyl]phenyl]-1H-pyridin-4- one 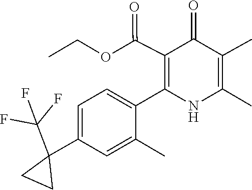 [SMILES: CCOC(=O)c1c(-c2ccc(C3(C(F)(F)F)CC3)cc2C)[nH]c(C)c(C)c1=O]
ethyl 5,6-dimethyl-2-[2-methyl-4-[1- (trifluoromethyl)cyclopropyl]phenyl]-4-oxo-1H- pyridine-3-carboxylate 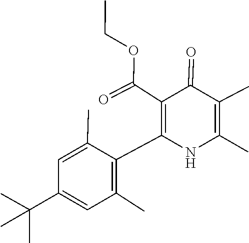 [SMILES: CCOC(=O)c1c(-c2c(C)cc(C(C)(C)C)cc2C)[nH]c(C)c(C)c1=O]
ethyl 2-(4-tert-butyl-2,6-dimethyl-phenyl)-5,6- dimethyl-4-oxo-1H-pyridine-3-carboxylate 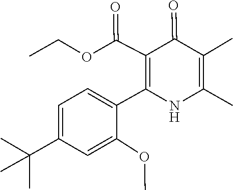 [SMILES: CCOC(=O)c1c(-c2ccc(C(C)(C)C)cc2OC)[nH]c(C)c(C)c1=O]
ethyl 2-(4-tert-butyl-2-methoxy-phenyl)-5,6- dimethyl-4-oxo-1H-pyridine-3-carboxylate 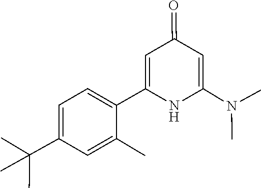 [SMILES: Cc1cc(C(C)(C)C)ccc1-c1cc(=O)cc(N(C)C)[nH]1]
2-(4-tert-butyl-2-methyl-phenyl)-6- (dimethylamino)-1H-pyridin-4-one 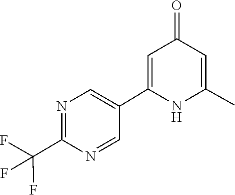 [SMILES: Cc1cc(=O)cc(-c2cnc(C(F)(F)F)nc2)[nH]1]
2-methyl-6-[2-(trifluoromethyl)pyrimidin-5-yl]-1H- pyridin-4-one 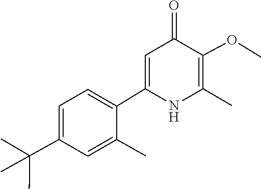 [SMILES: COc1c(C)[nH]c(-c2ccc(C(C)(C)C)cc2C)cc1=O]
6-(4-tert-butyl-2-methyl-phenyl)-3-methoxy-2- methyl-1H-pyridin-4-one 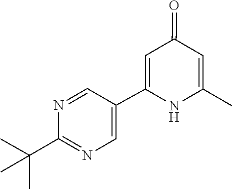 [SMILES: Cc1cc(=O)cc(-c2cnc(C(C)(C)C)nc2)[nH]1]
2-(2-tert-butylpyrimidin-5-yl)-6-methyl-1H-pyridin- 4-one 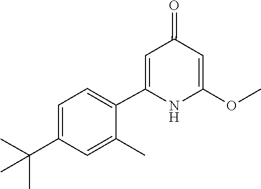 [SMILES: COc1cc(=O)cc(-c2ccc(C(C)(C)C)cc2C)[nH]1]
2-(4-tert-butyl-2-methyl-phenyl)-6-methoxy-1H- pyridin-4-one 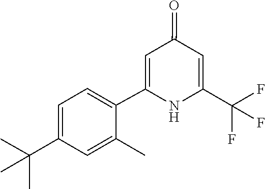 [SMILES: Cc1cc(C(C)(C)C)ccc1-c1cc(=O)cc(C(F)(F)F)[nH]1]
2-(4-tert-butyl-2-methyl-phenyl)-6- (trifluoromethyl)-1H-pyridin-4-one 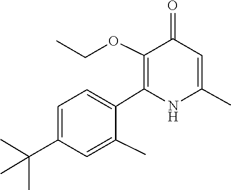 [SMILES: CCOc1c(-c2ccc(C(C)(C)C)cc2C)[nH]c(C)cc1=O]
2-(4-tert-butyl-2-methyl-phenyl)-3-ethoxy-6- methyl-1H-pyridin-4-one 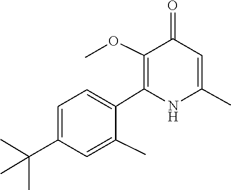 [SMILES: COc1c(-c2ccc(C(C)(C)C)cc2C)[nH]c(C)cc1=O]
2-(4-tert-butyl-2-methyl-phenyl)-3-methoxy-6- methyl-1H-pyridin-4-one 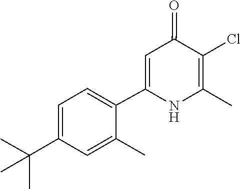 [SMILES: Cc1cc(C(C)(C)C)ccc1-c1cc(=O)c(Cl)c(C)[nH]1]
6-(4-tert-butyl-2-methyl-phenyl)-3-chloro-2-methyl- 1H-pyridin-4-one 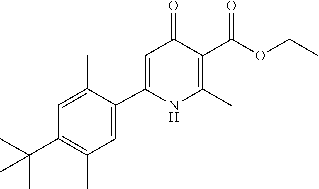 [SMILES: CCOC(=O)c1c(C)[nH]c(-c2cc(C)c(C(C)(C)C)cc2C)cc1=O]
ethyl 6-(4-tert-butyl-2,5-dimethyl-phenyl)-2-methyl- 4-oxo-1H-pyridine-3-carboxylate 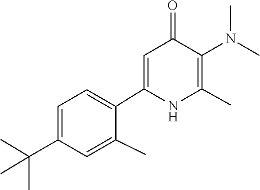 [SMILES: Cc1cc(C(C)(C)C)ccc1-c1cc(=O)c(N(C)C)c(C)[nH]1]
6-(4-tert-butyl-2-methyl-phenyl)-3- (dimethylamino)-2-methyl-1H-pyridin-4-one 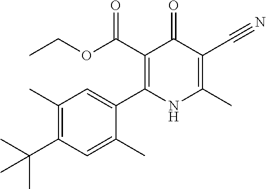 [SMILES: CCOC(=O)c1c(-c2cc(C)c(C(C)(C)C)cc2C)[nH]c(C)c(C#N)c1=O]
ethyl 2-(4-tert-butyl-2,5-dimethyl-phenyl)-5-cyano- 6-methyl-4-oxo-1H-pyridine-3-carboxylate 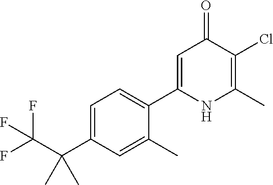 [SMILES: Cc1cc(C(C)(C)C(F)(F)F)ccc1-c1cc(=O)c(Cl)c(C)[nH]1]
3-chloro-2-methyl-6-[2-methyl-4-(2,2,2-trifluoro- 1,1-dimethyl-ethyl)phenyl]-1H-pyridin-4-one 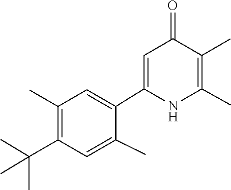 [SMILES: Cc1cc(C(C)(C)C)c(C)cc1-c1cc(=O)c(C)c(C)[nH]1]
6-(4-tert-butyl-2,5-dimethyl-phenyl)-2,3-dimethyl- 1H-pyridin-4-one 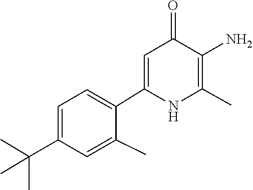 [SMILES: Cc1cc(C(C)(C)C)ccc1-c1cc(=O)c(N)c(C)[nH]1]
3-amino-6-(4-tert-butyl-2-methyl-phenyl)-2-methyl- 1H-pyridin-4-one 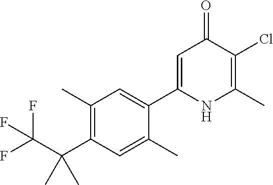 [SMILES: Cc1cc(C(C)(C)C(F)(F)F)c(C)cc1-c1cc(=O)c(Cl)c(C)[nH]1]
3-chloro-6-[2,5-dimethyl-4-(2,2,2-trifluoro-1,1- dimethyl-ethyl)phenyl]-2-methyl-1H-pyridin-4-one 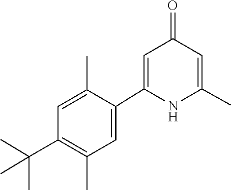 [SMILES: Cc1cc(=O)cc(-c2cc(C)c(C(C)(C)C)cc2C)[nH]1]
2-(4-tert-butyl-2,5-dimethyl-phenyl)-6-methyl-1H- pyridin-4-one 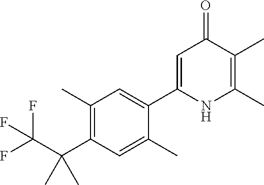 [SMILES: Cc1cc(C(C)(C)C(F)(F)F)c(C)cc1-c1cc(=O)c(C)c(C)[nH]1]
6-[2,5-dimethyl-4-(2,2,2-trifluoro-1,1-dimethyl- ethyl)phenyl]-2,3-dimethyl-1H-pyridin-4-one 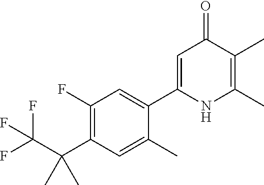 [SMILES: Cc1cc(C(C)(C)C(F)(F)F)c(F)cc1-c1cc(=O)c(C)c(C)[nH]1]
6-[5-fluoro-2-methyl-4-(2,2,2-trifluoro-1,1- dimethyl-ethyl)phenyl]-2,3-dimethyl-1H-pyridin-4- one 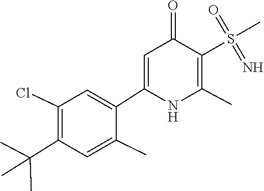 [SMILES: Cc1cc(C(C)(C)C)c(Cl)cc1-c1cc(=O)c(S(C)(=N)=O)c(C)[nH]1]
6-(4-tert-butyl-5-chloro-2-methyl-phenyl)-2-methyl- 3-(methylsulfonimidoyl)-1H-pyridin-4-one 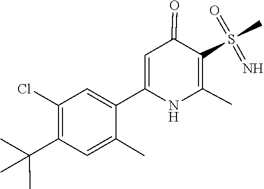 [SMILES: Cc1cc(C(C)(C)C)c(Cl)cc1-c1cc(=O)c([S@@](C)(=N)=O)c(C)[nH]1]
(S)-6-(4-tert-butyl-5-chloro-2-methyl-phenyl)-2- methyl-3-(methylsulfonimidoyl)-1H-pyridin-4-one 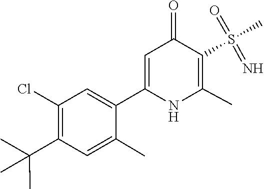 [SMILES: Cc1cc(C(C)(C)C)c(Cl)cc1-c1cc(=O)c([S@](C)(=N)=O)c(C)[nH]1]
(R)-6-(4-tert-butyl-5-chloro-2-methyl-phenyl)-2- methyl-3-(methylsulfonimidoyl)-1H-pyridin-4-one 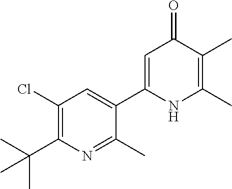 [SMILES: Cc1nc(C(C)(C)C)c(Cl)cc1-c1cc(=O)c(C)c(C)[nH]1]
6-(6-tert-butyl-5-chloro-2-methyl-3-pyridyl)-2,3- dimethyl-1H-pyridin-4-one 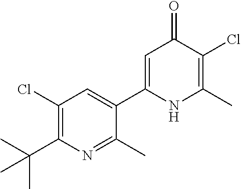 [SMILES: Cc1nc(C(C)(C)C)c(Cl)cc1-c1cc(=O)c(Cl)c(C)[nH]1]
6-(6-tert-butyl-5-chloro-2-methyl-3-pyridyl)-3- chloro-2-methyl-1H-pyridin-4-one 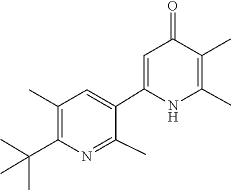 [SMILES: Cc1cc(-c2cc(=O)c(C)c(C)[nH]2)c(C)nc1C(C)(C)C]
6-(6-tert-butyl-2,5-dimethyl-3-pyridyl)-2,3- dimethyl-1H-pyridin-4-one 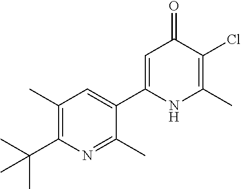 [SMILES: Cc1cc(-c2cc(=O)c(Cl)c(C)[nH]2)c(C)nc1C(C)(C)C]
6-(6-tert-butyl-2,5-dimethyl-3-pyridyl)-3-chloro-2- methyl-1H-pyridin-4-one 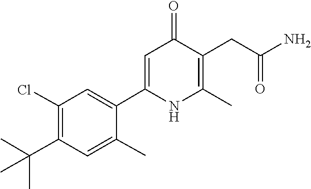 [SMILES: Cc1cc(C(C)(C)C)c(Cl)cc1-c1cc(=O)c(CC(N)=O)c(C)[nH]1]
2-[6-(4-tert-butyl-5-chloro-2-methyl-phenyl)-2- methyl-4-oxo-1H-pyridin-3-yl]acetamide 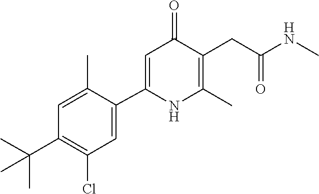 [SMILES: CNC(=O)Cc1c(C)[nH]c(-c2cc(Cl)c(C(C)(C)C)cc2C)cc1=O]
2-[6-(4-tert-butyl-5-chloro-2-methyl-phenyl)-2- methyl-4-oxo-1H-pyridin-3-yl]-N-methyl-acetamide 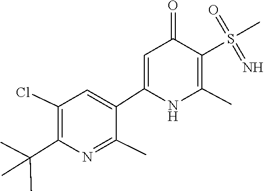 [SMILES: Cc1nc(C(C)(C)C)c(Cl)cc1-c1cc(=O)c(S(C)(=N)=O)c(C)[nH]1]
6-(6-tert-butyl-5-chloro-2-methyl-3-pyridyl)-2- methyl-3-(methylsulfonimidoyl)-1H-pyridin-4-one 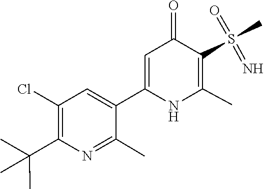 [SMILES: Cc1nc(C(C)(C)C)c(Cl)cc1-c1cc(=O)c([S@@](C)(=N)=O)c(C)[nH]1]
(S)-6-(6-tert-butyl-5-chloro-2-methyl-3-pyridyl)-2- methyl-3-(methylsulfonimidoyl)-1H-pyridin-4-one 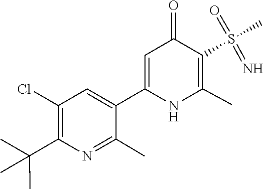 [SMILES: Cc1nc(C(C)(C)C)c(Cl)cc1-c1cc(=O)c([S@](C)(=N)=O)c(C)[nH]1]
(R)-6-(6-tert-butyl-5-chloro-2-methyl-3-pyridyl)-2- methyl-3-(methylsulfonimidoyl)-1H-pyridin-4-one 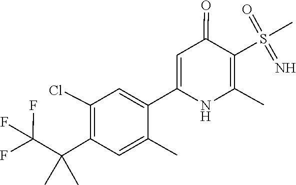 [SMILES: Cc1cc(C(C)(C)C(F)(F)F)c(Cl)cc1-c1cc(=O)c(S(C)(=N)=O)c(C)[nH]1]
6-[5-chloro-2-methyl-4-(2,2,2-trifluoro-1,1- dimethyl-ethyl)phenyl]-2-methyl-3- (methylsulfonimidoyl)-1H-pyridin-4-one 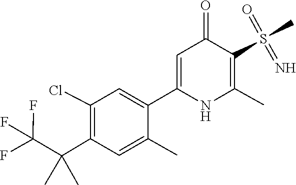 [SMILES: Cc1cc(C(C)(C)C(F)(F)F)c(Cl)cc1-c1cc(=O)c([S@@](C)(=N)=O)c(C)[nH]1]
(S)-6-[5-chloro-2-methyl-4-(2,2,2-trifluoro-1,1- dimethyl-ethyl)phenyl]-2-methyl-3- (methylsulfonimidoyl)-1H-pyridin-4-one 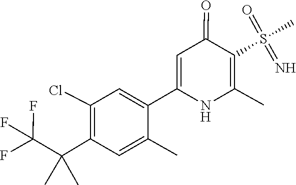 [SMILES: Cc1cc(C(C)(C)C(F)(F)F)c(Cl)cc1-c1cc(=O)c([S@](C)(=N)=O)c(C)[nH]1]
(R)-6-[5-chloro-2-methyl-4-(2,2,2-trifluoro-1,1- dimethyl-ethyl)phenyl]-2-methyl-3- (methylsulfonimidoyl)-1H-pyridin-4-one 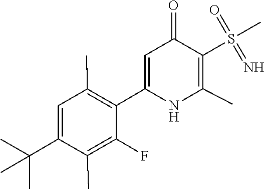 [SMILES: Cc1cc(C(C)(C)C)c(C)c(F)c1-c1cc(=O)c(S(C)(=N)=O)c(C)[nH]1]
6-(4-tert-butyl-2-fluoro-3,6-dimethyl-phenyl)-2- methyl-3-(methylsulfonimidoyl)-1H-pyridin-4-one 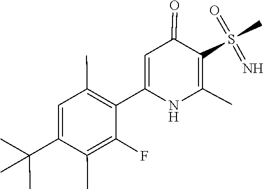 [SMILES: Cc1cc(C(C)(C)C)c(C)c(F)c1-c1cc(=O)c([S@@](C)(=N)=O)c(C)[nH]1]
(S)-6-(4-tert-butyl-2-fluoro-3,6-dimethyl-phenyl)-2- methyl-3-(methylsulfonimidoyl)-1H-pyridin-4-one 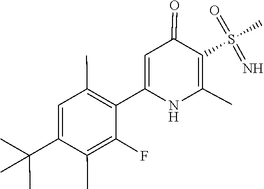 [SMILES: Cc1cc(C(C)(C)C)c(C)c(F)c1-c1cc(=O)c([S@](C)(=N)=O)c(C)[nH]1]
(R)-6-(4-tert-butyl-2-fluoro-3,6-dimethyl-phenyl)-2- methyl-3-(methylsulfonimidoyl)-1/-pyridin-4-one 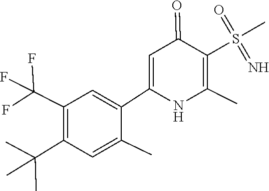 [SMILES: Cc1cc(C(C)(C)C)c(C(F)(F)F)cc1-c1cc(=O)c(S(C)(=N)=O)c(C)[nH]1]
6-[4-tert-butyl-2-methyl-5-(trifluoromethyl)phenyl]- 2-methyl-3-(methylsulfonimidoyl)-1H-pyridin-4- one 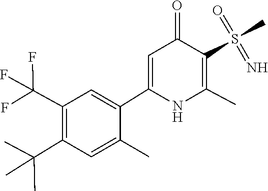 [SMILES: Cc1cc(C(C)(C)C)c(C(F)(F)F)cc1-c1cc(=O)c([S@@](C)(=N)=O)c(C)[nH]1]
(S)-6-[4-tert-butyl-2-methyl-5- (trifluoromethyl)phenyl]-2-methyl-3- (methylsulfonimidoyl)-1H-pyridin-4-one 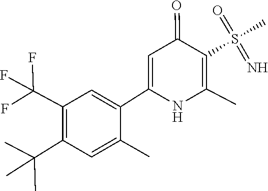 [SMILES: Cc1cc(C(C)(C)C)c(C(F)(F)F)cc1-c1cc(=O)c([S@](C)(=N)=O)c(C)[nH]1]
(R)-6-[4-tert-butyl-2-methyl-5- (trifluoromethyl)phenyl]-2-methyl-3- (methylsulfonimidoyl)-1H-pyridin-4-one 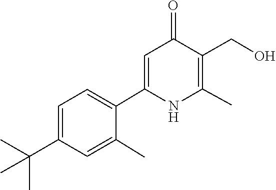 [SMILES: Cc1cc(C(C)(C)C)ccc1-c1cc(=O)c(CO)c(C)[nH]1]
6-(4-tert-butyl-2-methyl-phenyl)-3- (hydroxymethyl)-2-methyl-1H-pyridin-4-one 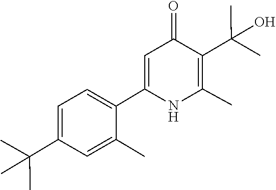 [SMILES: Cc1cc(C(C)(C)C)ccc1-c1cc(=O)c(C(C)(C)O)c(C)[nH]1]
6-(4-tert-butyl-2-methyl-phenyl)-3-(1-hydroxy-1- methyl-ethyl)-2-methyl-1H-pyridin-4-one 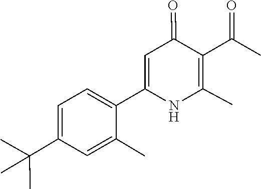 [SMILES: CC(=O)c1c(C)[nH]c(-c2ccc(C(C)(C)C)cc2C)cc1=O]
3-acetyl-6-(4-tert-butyl-2-methyl-phenyl)-2-methyl- 1H-pyridin-4-one 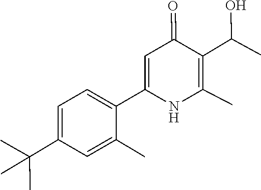 [SMILES: Cc1cc(C(C)(C)C)ccc1-c1cc(=O)c(C(C)O)c(C)[nH]1]
6-(4-tert-butyl-2-methyl-phenyl)-3-(1- hydroxyethyl)-2-methyl-1H-pyridin-4-one 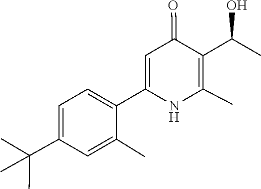 [SMILES: Cc1cc(C(C)(C)C)ccc1-c1cc(=O)c([C@H](C)O)c(C)[nH]1]
(S)-6-(4-tert-butyl-2-methyl-phenyl)-3-(1- hydroxyethyl)-2-methyl-1H-pyridin-4-one 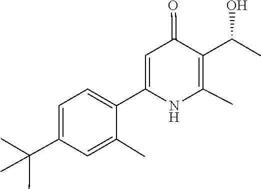 [SMILES: Cc1cc(C(C)(C)C)ccc1-c1cc(=O)c([C@@H](C)O)c(C)[nH]1]
(R)-6-(4-tert-butyl-2-methyl-phenyl)-3-(1- hydroxyethyl)-2-methyl-1H-pyridin-4-one 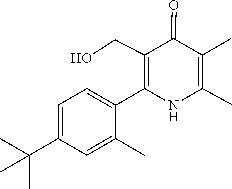 [SMILES: Cc1cc(C(C)(C)C)ccc1-c1[nH]c(C)c(C)c(=O)c1CO]
2-(4-tert-butyl-2-methyl-phenyl)-3- (hydroxymethyl)-5,6-dimethyl-1H-pyridin-4-one 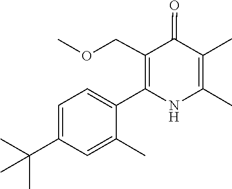 [SMILES: COCc1c(-c2ccc(C(C)(C)C)cc2C)[nH]c(C)c(C)c1=O]
2-(4-tert-butyl-2-methyl-phenyl)-3- (methoxymethyl)-5,6-dimethyl-1H-pyridin-4-one 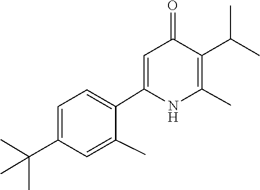 [SMILES: Cc1cc(C(C)(C)C)ccc1-c1cc(=O)c(C(C)C)c(C)[nH]1]
6-(4-tert-butyl-2-methyl-phenyl)-3-isopropyl-2- methyl-1H-pyridin-4-one 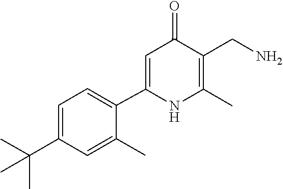 [SMILES: Cc1cc(C(C)(C)C)ccc1-c1cc(=O)c(CN)c(C)[nH]1]
3-(aminomethyl)-6-(4-tert-butyl-2-methyl-phenyl)- 2-methyl-1H-pyridin-4-one 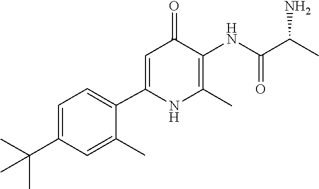 [SMILES: Cc1cc(C(C)(C)C)ccc1-c1cc(=O)c(NC(=O)[C@@H](C)N)c(C)[nH]1]
(2R)-2-amino-N-[6-(4-tert-butyl-2-methyl-phenyl)- 2-methyl-4-oxo-1H-pyridin-3-yl]propanamide 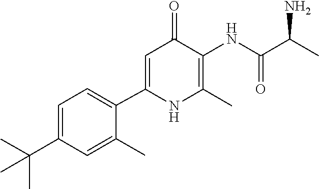 [SMILES: Cc1cc(C(C)(C)C)ccc1-c1cc(=O)c(NC(=O)[C@H](C)N)c(C)[nH]1]
(2S)-2-amino-N-[6-(4-tert-butyl-2-methyl-phenyl)- 2-methyl-4-oxo-1H-pyridin-3-yl]propanamide 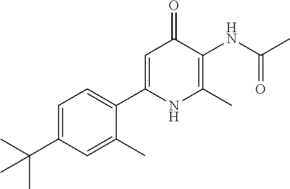 [SMILES: CC(=O)Nc1c(C)[nH]c(-c2ccc(C(C)(C)C)cc2C)cc1=O]
N-[6-(4-tert-butyl-2-methyl-phenyl)-2-methyl-4- oxo-1H-pyridin-3-yl]acetamide 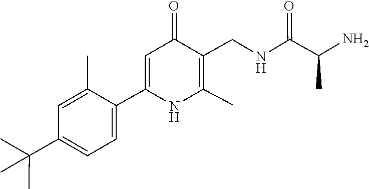 [SMILES: Cc1cc(C(C)(C)C)ccc1-c1cc(=O)c(CNC(=O)[C@H](C)N)c(C)[nH]1]
(2S)-2-amino-N-[[6-(4-tert-butyl-2-methyl-phenyl)- 2-methyl-4-oxo-1H-pyridin-3- yl]methyl]propanamide 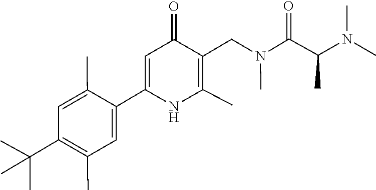 [SMILES: Cc1cc(C(C)(C)C)c(C)cc1-c1cc(=O)c(CN(C)C(=O)[C@H](C)N(C)C)c(C)[nH]1]
(2S)-N-[[6-(4-tert-butyl-2,5-dimethyl-phenyl)-2- methyl-4-oxo-1H-pyridin-3-yl]methyl]-2- (dimethylamino)-N-methyl-propanamide 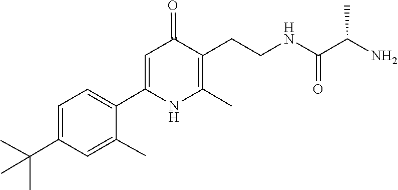 [SMILES: Cc1cc(C(C)(C)C)ccc1-c1cc(=O)c(CCNC(=O)[C@H](C)N)c(C)[nH]1]
(2S)-2-amino-N-[2-[6-(4-tert-butyl-2-methyl- phenyl)-2-methyl-4-oxo-1H-pyridin-3- yl]ethyl]propanamide 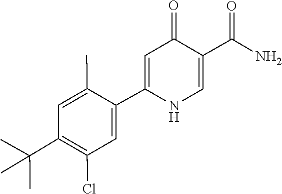 [SMILES: Cc1cc(C(C)(C)C)c(Cl)cc1-c1cc(=O)c(C(N)=O)c[nH]1]
6-(4-(tert-butyl)-5-chloro-2-methylphenyl)-4-oxo- 1,4-dihydropyridine-3-carboxamide 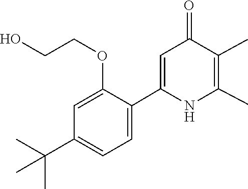 [SMILES: Cc1[nH]c(-c2ccc(C(C)(C)C)cc2OCCO)cc(=O)c1C]
6-[4-tert-butyl-2-(2-hydroxyethoxy)phenyl]-2,3- dimethyl-1H-pyridin-4-one 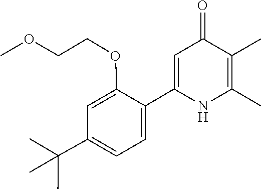
6-[4-tert-butyl-2-(2-methoxyethoxy)phenyl]-2,3- dimethyl-1H-pyridin-4-one 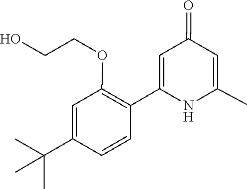 [SMILES: Cc1cc(=O)cc(-c2ccc(C(C)(C)C)cc2OCCO)[nH]1]
2-[4-tert-butyl-2-(2-hydroxyethoxy)phenyl]-6- methyl-1H-pyridin-4-one 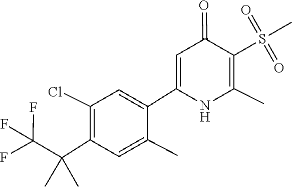 [SMILES: Cc1cc(C(C)(C)C(F)(F)F)c(Cl)cc1-c1cc(=O)c(S(C)(=O)=O)c(C)[nH]1]
6-[5-chloro-2-methyl-4-(2,2,2-trifluoro-1,1- dimethyl-ethyl)phenyl]-2-methyl-3-methylsulfonyl- 1H-pyridin-4-one 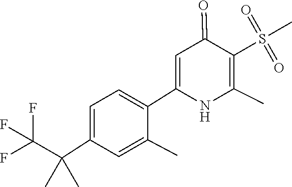 [SMILES: Cc1cc(C(C)(C)C(F)(F)F)ccc1-c1cc(=O)c(S(C)(=O)=O)c(C)[nH]1]
2-methyl-3-methylsulfonyl-6-[2-methyl-4-(2,2,2- trifluoro-1,1-dimethyl-ethyl)phenyl]-1H-pyridin-4- one 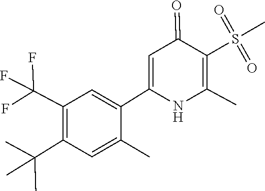 [SMILES: Cc1cc(C(C)(C)C)c(C(F)(F)F)cc1-c1cc(=O)c(S(C)(=O)=O)c(C)[nH]1]
6-[4-tert-butyl-2-methyl-5-(trifluoromethyl)phenyl]-2- methyl-3-methylsulfonyl-1H-pyridin-4-one

In some embodiments, the invention relates to a compound selected from Table B, or a pharmaceutically acceptable salt thereof. In other embodiments, the invention relates to a compound selected from Table B, i.e., the compound in non-salt form.

TABLE B
Compound Structures and Names.

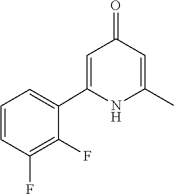 [SMILES: Cc1cc(=O)cc(-c2cccc(F)c2F)[nH]1]

2-(2,3-difluorophenyl)-6-methyl-1H-pyridin-4-one

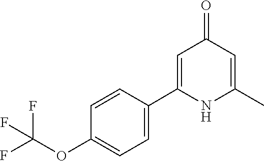 [SMILES: Cc1cc(=O)cc(-c2ccc(OC(F)(F)F)cc2)[nH]1]

2-methyl-6-[4-(trifluoromethoxy)phenyl]-1H-
pyridin-4-one

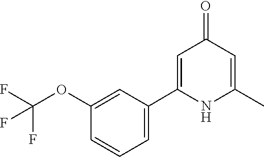 [SMILES: Cc1cc(=O)cc(-c2cccc(OC(F)(F)F)c2)[nH]1]

2-methyl-6-[3-(trifluoromethoxy)phenyl]-1H-
pyridin-4-one

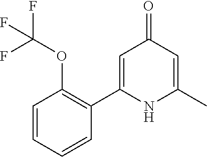 [SMILES: Cc1cc(=O)cc(-c2ccccc2OC(F)(F)F)[nH]1]

2-methyl-6-[2-(trifluoromethoxy)phenyl]-1H-
pyridin-4-one

In some embodiments, the invention relates to a compound selected from Table C, or a pharmaceutically acceptable salt thereof. In other embodiments, the invention relates to a compound selected from Table C, i.e., the compound in non-salt form.

TABLE C
Compound Structures and Names.

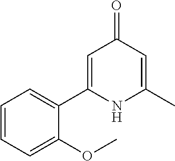 [SMILES: COc1ccccc1-c1cc(=O)cc(C)[nH]1]

2-(2-methoxyphenyl)-6-methyl-1H-pyridin-4-one

In some embodiments, the invention relates to a compound selected from Table D, or a pharmaceutically acceptable salt thereof. In other embodiments, the invention relates to a compound selected from Table C, i.e., the compound in non-salt form.

TABLE D
Compound Structures and Names.

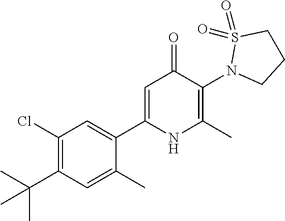 [SMILES: Cc1cc(C(C)(C)C)c(Cl)cc1-c1cc(=O)c(N2CCCS2(=O)=O)c(C)[nH]1]

6-(4-tert-butyl-5-chloro-2-methyl-phenyl)-3-(1,1-
dioxo-1,2-thiazolidin-2-yl)-2-methyl-1H-pyridin-4-
one

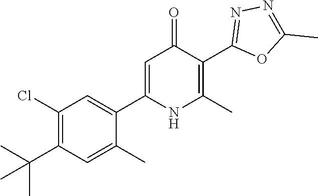 [SMILES: Cc1nnc(-c2c(C)[nH]c(-c3cc(Cl)c(C(C)(C)C)cc3C)cc2=O)o1]

6-(4-tert-butyl-5-chloro-2-methyl-phenyl)-2-methyl-
3-(5-methyl-1,3,4-oxadiazol-2-yl)-1H-pyridin-4-one

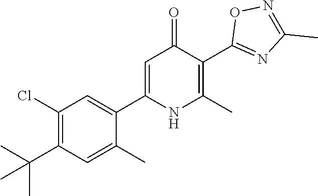 [SMILES: Cc1noc(-c2c(C)[nH]c(-c3cc(Cl)c(C(C)(C)C)cc3C)cc2=O)n1]

6-(4-tert-butyl-5-chloro-2-methyl-phenyl)-2-methyl-
3-(3-methyl-1,2,4-oxadiazol-5-yl)-1H-pyridin-4-one

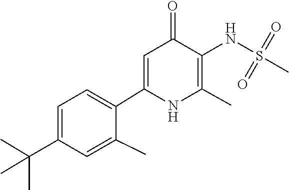 [SMILES: Cc1cc(C(C)(C)C)ccc1-c1cc(=O)c(NS(C)(=O)=O)c(C)[nH]1]

N-[6-(4-tert-butyl-2-methyl-phenyl)-2-methyl-4-
oxo-1H-pyridin-3-yl]methanesulfonamide

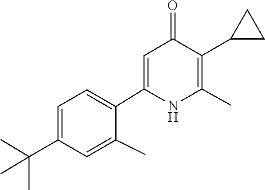 [SMILES: Cc1cc(C(C)(C)C)ccc1-c1cc(=O)c(C2CC2)c(C)[nH]1]

6-(4-tert-butyl-2,5-dimethyl-phenyl)-3-cyclopropyl- 2-methyl-1H-pyridin-4-one

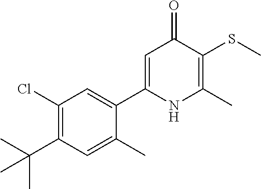 [SMILES: CSc1c(C)[nH]c(-c2cc(Cl)c(C(C)(C)C)cc2C)cc1=O]

6-(4-tert-butyl-5-chloro-2-methyl-phenyl)-2-methyl-
3-methylsulfanyl-1H-pyridin-4-one

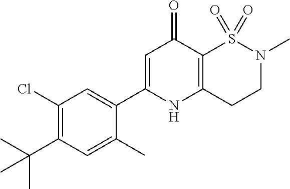 [SMILES: Cc1cc(C(C)(C)C)c(Cl)cc1-c1cc(=O)c2c([nH]1)CCN(C)S2(=O)=O]

6-(4-tert-butyl-5-chloro-2-methyl-phenyl)-2-methyl-
1,1-dioxo-4,5-dihydro-3H-pyrido[2,3-e]thiazin-8-
one

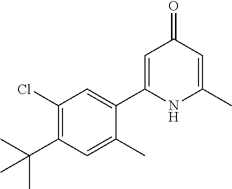 [SMILES: Cc1cc(=O)cc(-c2cc(Cl)c(C(C)(C)C)cc2C)[nH]1]

2-(4-tert-butyl-5-chloro-2-methyl-phenyl)-6-methyl-
1H-pyridin-4-one

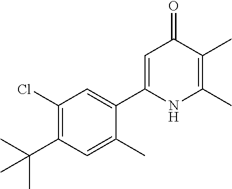 [SMILES: Cc1cc(C(C)(C)C)c(Cl)cc1-c1cc(=O)c(C)c(C)[nH]1]

6-(4-tert-butyl-5-chloro-2-methyl-phenyl)-2,3-
dimethyl-1H-pyridin-4-one

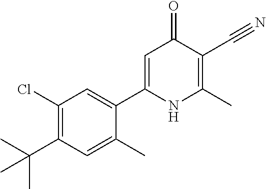 [SMILES: Cc1cc(C(C)(C)C)c(Cl)cc1-c1cc(=O)c(C#N)c(C)[nH]1]

6-(4-tert-butyl-5-chloro-2-methyl-phenyl)-2-methyl-
4-oxo-1H-pyridine-3-carbonitrile

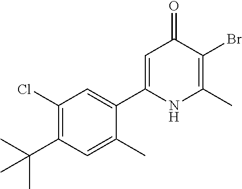 [SMILES: Cc1cc(C(C)(C)C)c(Cl)cc1-c1cc(=O)c(Br)c(C)[nH]1]

3-bromo-6-(4-tert-butyl-5-chloro-2-methyl-phenyl)-
2-methyl-1H-pyridin-4-one

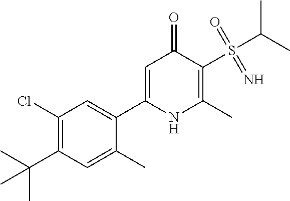 [SMILES: Cc1cc(C(C)(C)C)c(Cl)cc1-c1cc(=O)c(S(=N)(=O)C(C)C)c(C)[nH]1]

6-(4-tert-butyl-5-chloro-2-methyl-phenyl)-3-
(isopropylsulfonimidoyl)-2-methyl-1H-pyridin-4-
one

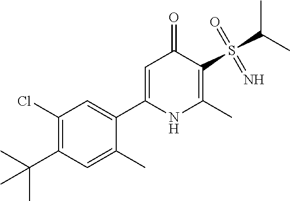 [SMILES: Cc1cc(C(C)(C)C)c(Cl)cc1-c1cc(=O)c([S@@](=N)(=O)C(C)C)c(C)[nH]1]

(S)-6-(4-(tert-butyl)-5-chloro-2-methylphenyl)-2-
methyl-3-(propan-2-ylsulfonimidoyl)pyridin-4(1H)-
one

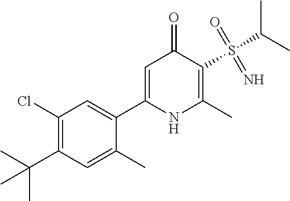 [SMILES: Cc1cc(C(C)(C)C)c(Cl)cc1-c1cc(=O)c([S@](=N)(=O)C(C)C)c(C)[nH]1]

(R)-6-(4-(tert-butyl)-5-chloro-2-methylphenyl)-2-
methyl-3-(propan-2-ylsulfonimidoyl)pyridin-4(1H)-
one

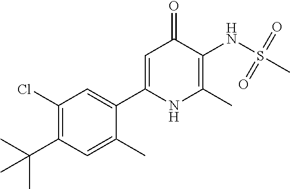 [SMILES: Cc1cc(C(C)(C)C)c(Cl)cc1-c1cc(=O)c(NS(C)(=O)=O)c(C)[nH]1]

N-[6-(4-tert-butyl-5-chloro-2-methyl-phenyl)-2-
methyl-4-oxo-1H-pyridin-3-yl]methanesulfonamide

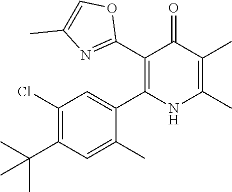 [SMILES: Cc1coc(-c2c(-c3cc(Cl)c(C(C)(C)C)cc3C)[nH]c(C)c(C)c2=O)n1]

2-(4-tert-butyl-5-chloro-2-methyl-phenyl)-5,6-
dimethyl-3-(4-methyloxazol-2-yl)-1H-pyridin-4-one

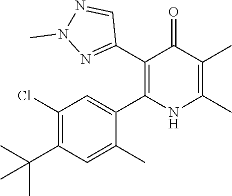 [SMILES: Cc1cc(C(C)(C)C)c(Cl)cc1-c1[nH]c(C)c(C)c(=O)c1-c1cnn(C)n1]

2-(4-tert-butyl-5-chloro-2-methyl-phenyl)-5,6-
dimethyl-3-(2-methyltriazol-4-yl)-1H-pyridin-4-one

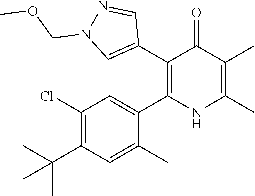

2-(4-tert-butyl-5-chloro-2-methyl-phenyl)-3-[1-
(methoxymethyl)pyrazol-4-yl]-5,6-dimethyl-1H-
pyridin-4-one

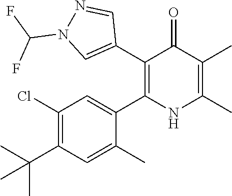 [SMILES: Cc1cc(C(C)(C)C)c(Cl)cc1-c1[nH]c(C)c(C)c(=O)c1-c1cnn(C(F)F)c1]

2-(4-tert-butyl-5-chloro-2-methyl-phenyl)-3-[1-
(difluoromethyl)pyrazol-3-yl]-5,6-dimethyl-1H-
pyridin-4-one

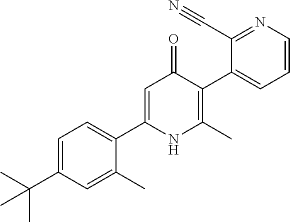 [SMILES: Cc1cc(C(C)(C)C)ccc1-c1cc(=O)c(-c2cccnc2C#N)c(C)[nH]1]

3-[6-(4-tert-butyl-2-methyl-phenyl)-2-methyl-4-
oxo-1H-pyridin-3-yl]pyridine-2-carbonitrile

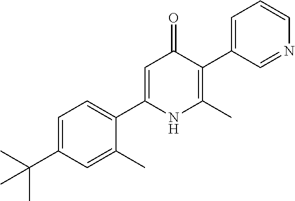 [SMILES: Cc1cc(C(C)(C)C)ccc1-c1cc(=O)c(-c2cccnc2)c(C)[nH]1]

6-(4-tert-butyl-2-methyl-phenyl)-2-methyl-3-(3-
pyridyl)-1H-pyridin-4-one

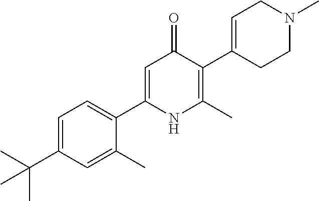 [SMILES: Cc1cc(C(C)(C)C)ccc1-c1cc(=O)c(C2=CCN(C)CC2)c(C)[nH]1]

6-(4-tert-butyl-2-methyl-phenyl)-2-methyl-3-(1-
methyl-3,6-dihydro-2H-pyridin-4-yl)-1H-pyridin-4-
one

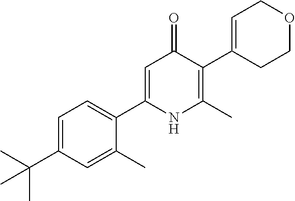 [SMILES: Cc1cc(C(C)(C)C)ccc1-c1cc(=O)c(C2=CCOCC2)c(C)[nH]1]

6-(4-tert-butyl-2-methyl-phenyl)-3-(3,6-dihydro-2H-
pyran-4-yl)-2-methyl-1H-pyridin-4-one

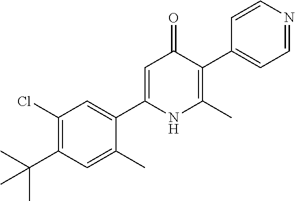 [SMILES: Cc1cc(C(C)(C)C)c(Cl)cc1-c1cc(=O)c(-c2ccncc2)c(C)[nH]1]

6-(4-tert-butyl-5-chloro-2-methyl-phenyl)-2-methyl-
3-(4-pyridyl)-1H-pyridin-4-one

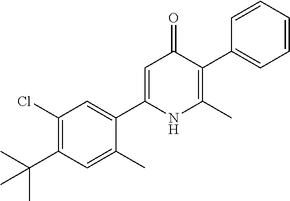 [SMILES: Cc1cc(C(C)(C)C)c(Cl)cc1-c1cc(=O)c(-c2ccccc2)c(C)[nH]1]

6-(4-tert-butyl-5-chloro-2-methyl-phenyl)-2-methyl-
3-phenyl-1H-pyridin-4-one

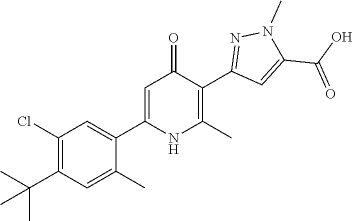 [SMILES: Cc1cc(C(C)(C)C)c(Cl)cc1-c1cc(=O)c(-c2cc(C(=O)O)n(C)n2)c(C)[nH]1]

5-[6-(4-tert-butyl-5-chloro-2-methyl-phenyl)-2-
methyl-4-oxo-1H-pyridin-3-yl]-2-methyl-pyrazole-
3-carboxylic acid

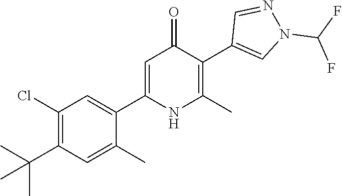 [SMILES: Cc1cc(C(C)(C)C)c(Cl)cc1-c1cc(=O)c(-c2cnn(C(F)F)c2)c(C)[nH]1]

6-(4-tert-butyl-5-chloro-2-methyl-phenyl)-3-[1-
(difluoromethyl)pyrazol-4-yl]-2-methyl-1H-pyridin-
4-one

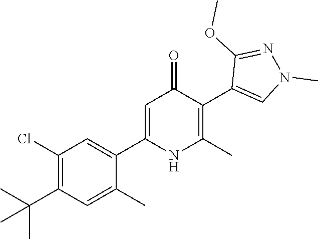

6-(4-tert-butyl-5-chloro-2-methyl-phenyl)-3-(3-
methoxy-1-methyl-pyrazol-4-yl)-2-methyl-1H-
pyridin-4-one

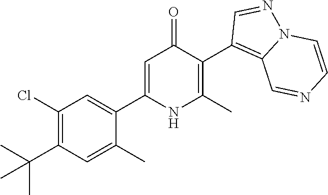 [SMILES: Cc1cc(C(C)(C)C)c(Cl)cc1-c1cc(=O)c(-c2cnn3ccncc23)c(C)[nH]1]

6-(4-tert-butyl-5-chloro-2-methyl-phenyl)-2-methyl-
3-pyrazolo[1,5-a]pyrazin-3-yl-1H-pyridin-4-one

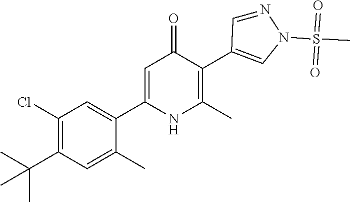 [SMILES: Cc1cc(C(C)(C)C)c(Cl)cc1-c1cc(=O)c(-c2cnn(S(C)(=O)=O)c2)c(C)[nH]1]

6-(4-tert-butyl-5-chloro-2-methyl-phenyl)-2-methyl-
3-(1-methylsulfonylpyrazol-4-yl)-1H-pyridin-4-one

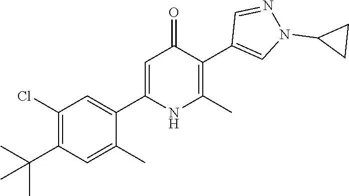

6-(4-tert-butyl-5-chloro-2-methyl-phenyl)-3-(1-
cyclopropylpyrazol-4-yl)-2-methyl-1H-pyridin-4-
one

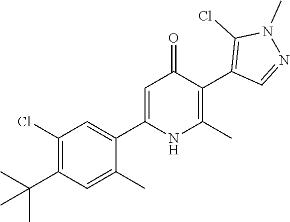

6-(4-tert-butyl-5-chloro-2-methyl-phenyl)-3-(5-
chloro-1-methyl-pyrazol-4-yl)-2-methyl-1H-pyridin-
4-one

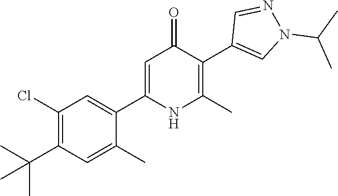

6-(4-tert-butyl-5-chloro-2-methyl-phenyl)-3-(1-
isopropylpyrazol-4-yl)-2-methyl-1H-pyridin-4-one

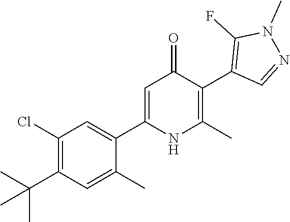 [SMILES: Cc1cc(C(C)(C)C)c(Cl)cc1-c1cc(=O)c(-c2cnn(C)c2F)c(C)[nH]1]

6-(4-tert-butyl-5-chloro-2-methyl-phenyl)-3-(5-
fluoro-1-methyl-pyrazol-4-yl)-2-methyl-1H-pyridin-
4-one

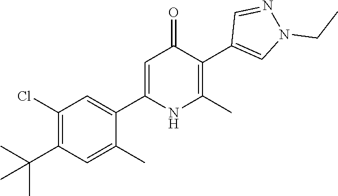 [SMILES: CCn1cc(-c2c(C)[nH]c(-c3cc(Cl)c(C(C)(C)C)cc3C)cc2=O)cn1]

6-(4-tert-butyl-5-chloro-2-methyl-phenyl)-3-(1-
ethylpyrazol-4-yl)-2-methyl-1H-pyridin-4-one

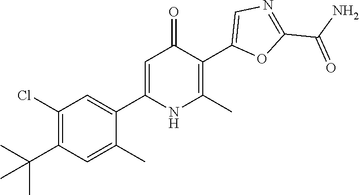 [SMILES: Cc1cc(C(C)(C)C)c(Cl)cc1-c1cc(=O)c(-c2cnc(C(N)=O)o2)c(C)[nH]1]

5-[6-(4-tert-butyl-5-chloro-2-methyl-phenyl)-2-
methyl-4-oxo-1H-pyridin-3-yl]oxazole-2-
carboxamide

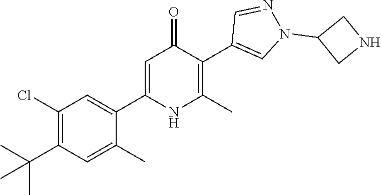

3-[1-(azetidin-3-yl)pyrazol-4-yl]-6-(4-tert-butyl-5-
chloro-2-methyl-phenyl)-2-methyl-1H-pyridin-4-
one

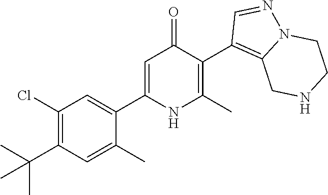 [SMILES: Cc1cc(C(C)(C)C)c(Cl)cc1-c1cc(=O)c(-c2cnn3c2CNCC3)c(C)[nH]1]

6-(4-tert-butyl-5-chloro-2-methyl-phenyl)-2-methyl-
3-(4,5,6,7-tetrahydropyrazolo[1,5-a]pyrazin-3-yl)-
1H-pyridin-4-one

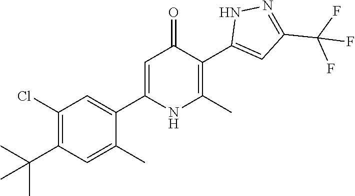 [SMILES: Cc1cc(C(C)(C)C)c(Cl)cc1-c1cc(=O)c(-c2cc(C(F)(F)F)n[nH]2)c(C)[nH]1]

6-(4-tert-butyl-5-chloro-2-methyl-phenyl)-2-methyl-
3-[3-(trifluoromethyl)-1H-pyrazol-5-yl]-1H-pyridin-
4-one

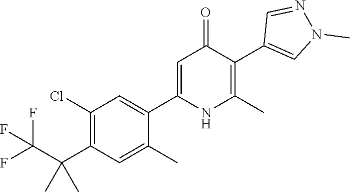

6-(5-chloro-2-methyl-4-(1,1,1-trifluoro-2-
methylpropan-2-yl)phenyl)-2-methyl-3-(1-methyl-
1H-pyrazol-4-yl)pyridin-4(1H)-one

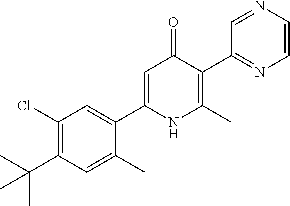 [SMILES: Cc1cc(C(C)(C)C)c(Cl)cc1-c1cc(=O)c(-c2cnccn2)c(C)[nH]1]

6-(4-tert-butyl-5-chloro-2-methyl-phenyl)-2-methyl-
3-pyrazin-2-yl-1H-pyridin-4-one

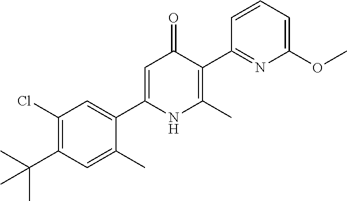 [SMILES: COc1cccc(-c2c(C)[nH]c(-c3cc(Cl)c(C(C)(C)C)cc3C)cc2=O)n1]

6-(4-tert-butyl-5-chloro-2-methyl-phenyl)-3-(6-
methoxy-2-pyridyl)-2-methyl-1H-pyridin-4-one

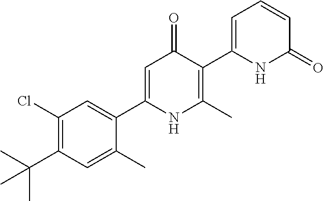 [SMILES: Cc1cc(C(C)(C)C)c(Cl)cc1-c1cc(=O)c(-c2cccc(=O)[nH]2)c(C)[nH]1]

6-(4-tert-butyl-5-chloro-2-methyl-phenyl)-2-methyl-
3-(6-oxo-1H-pyridin-2-yl)-1H-pyridin-4-one

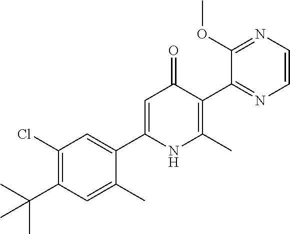 [SMILES: COc1nccnc1-c1c(C)[nH]c(-c2cc(Cl)c(C(C)(C)C)cc2C)cc1=O]

6-(4-tert-butyl-5-chloro-2-methyl-phenyl)-3-(3-
methoxypyrazin-2-yl)-2-methyl-1H-pyridin-4-one

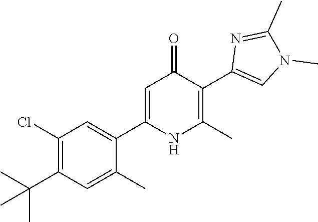 [SMILES: Cc1cc(C(C)(C)C)c(Cl)cc1-c1cc(=O)c(-c2cn(C)c(C)n2)c(C)[nH]1]

6-(4-tert-butyl-5-chloro-2-methyl-phenyl)-3-(1,2-
dimethylimidazol-4-yl)-2-methyl-1H-pyridin-4-one

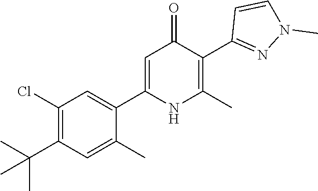 [SMILES: Cc1cc(C(C)(C)C)c(Cl)cc1-c1cc(=O)c(-c2ccn(C)n2)c(C)[nH]1]

6-(4-tert-butyl-5-chloro-2-methyl-phenyl)-2-methyl-
3-(1-methylpyrazol-3-yl)-1H-pyridin-4-one

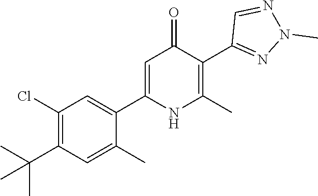 [SMILES: Cc1cc(C(C)(C)C)c(Cl)cc1-c1cc(=O)c(-c2cnn(C)n2)c(C)[nH]1]

6-(4-tert-butyl-5-chloro-2-methyl-phenyl)-2-methyl-
3-(2-methyltriazol-4-yl)-1H-pyridin-4-one

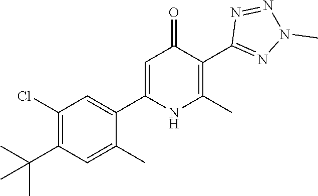 [SMILES: Cc1cc(C(C)(C)C)c(Cl)cc1-c1cc(=O)c(-c2nnn(C)n2)c(C)[nH]1]

6-(4-tert-butyl-5-chloro-2-methyl-phenyl)-2-methyl-
3-(2-methyltetrazol-5-yl)-1H-pyridin-4-one

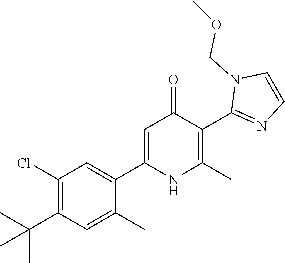 [SMILES: COCn1ccnc1-c1c(C)[nH]c(-c2cc(Cl)c(C(C)(C)C)cc2C)cc1=O]

6-(4-tert-butyl-5-chloro-2-methyl-phenyl)-3-[1-
(methoxymethyl)imidazol-2-yl]-2-methyl-1H-
pyridin-4-one

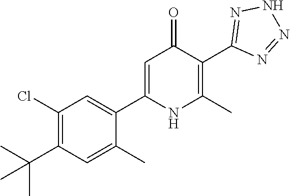 [SMILES: Cc1cc(C(C)(C)C)c(Cl)cc1-c1cc(=O)c(-c2nn[nH]n2)c(C)[nH]1]

6-(4-tert-butyl-5-chloro-2-methyl-phenyl)-2-methyl-
3-(2H-tetrazol-5-yl)-1H-pyridin-4-one

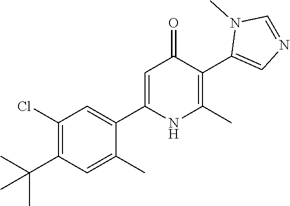 [SMILES: Cc1cc(C(C)(C)C)c(Cl)cc1-c1cc(=O)c(-c2cncn2C)c(C)[nH]1]

6-(4-(tert-butyl)-5-chloro-2-methylphenyl)-2-
methyl-3-(1-methyl-1H-imidazol-5-yl)pyridin-
4(1H)-one

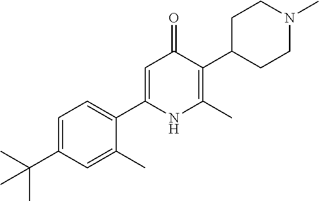 [SMILES: Cc1cc(C(C)(C)C)ccc1-c1cc(=O)c(C2CCN(C)CC2)c(C)[nH]1]

6-(4-tert-butyl-2-methyl-phenyl)-2-methyl-3-(1-
methyl-4-piperidyl)-1H-pyridin-4-one

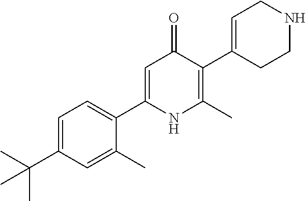 [SMILES: Cc1cc(C(C)(C)C)ccc1-c1cc(=O)c(C2=CCNCC2)c(C)[nH]1]

6-(4-tert-butyl-2-methyl-phenyl)-2-methyl-3-
(1,2,3,6-tetrahydropyridin-4-yl)-1H-pyridin-4-one

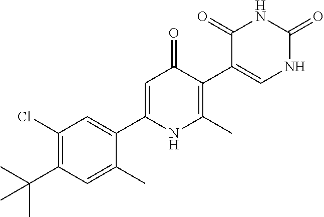 [SMILES: Cc1cc(C(C)(C)C)c(Cl)cc1-c1cc(=O)c(-c2c[nH]c(=O)[nH]c2=O)c(C)[nH]1]

5-[6-(4-tert-butyl-5-chloro-2-methyl-phenyl)-2-
methyl-4-oxo-1H-pyridin-3-yl]-1H-pyrimidine-2,4-
dione

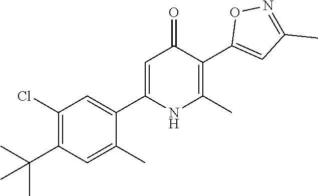 [SMILES: Cc1cc(-c2c(C)[nH]c(-c3cc(Cl)c(C(C)(C)C)cc3C)cc2=O)on1]

6-(4-tert-butyl-5-chloro-2-methyl-phenyl)-2-methyl-
3-(3-methylisoxazol-5-yl)-1H-pyridin-4-one

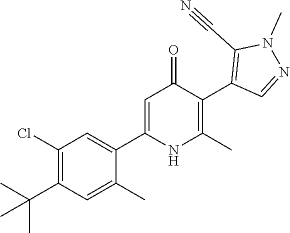 [SMILES: Cc1cc(C(C)(C)C)c(Cl)cc1-c1cc(=O)c(-c2cnn(C)c2C#N)c(C)[nH]1]

4-[6-(4-tert-butyl-5-chloro-2-methyl-phenyl)-2-
methyl-4-oxo-1H-pyridin-3-yl]-2-methyl-pyrazole-
3-carbonitrile

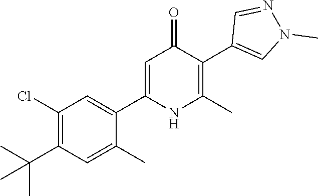 [SMILES: Cc1cc(C(C)(C)C)c(Cl)cc1-c1cc(=O)c(-c2cnn(C)c2)c(C)[nH]1]

6-(6-tert-butyl-5-chloro-2-methyl-3-pyridyl)-2-
methyl-3-(1-methylpyrazol-4-yl)-1H-pyridin-4-one

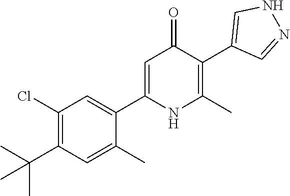 [SMILES: Cc1cc(C(C)(C)C)c(Cl)cc1-c1cc(=O)c(-c2cn[nH]c2)c(C)[nH]1]

6-(4-tert-butyl-5-chloro-2-methyl-phenyl)-2-methyl-
3-(1H-pyrazol-4-yl)-1H-pyridin-4-one

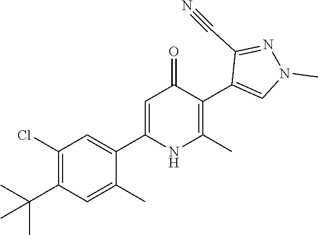

4-[6-(4-tert-butyl-5-chloro-2-methyl-phenyl)-2-
methyl-4-oxo-1H-pyridin-3-yl]-1-methyl-pyrazole-
3-carbonitrile

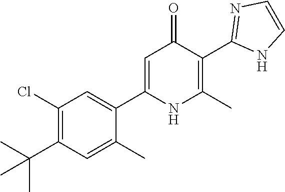 [SMILES: Cc1cc(C(C)(C)C)c(Cl)cc1-c1cc(=O)c(-c2ncc[nH]2)c(C)[nH]1]

6-(4-tert-butyl-5-chloro-2-methyl-phenyl)-3-(1H-
imidazol-2-yl)-2-methyl-1H-pyridin-4-one

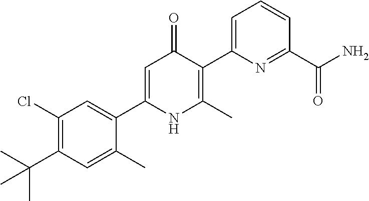 [SMILES: Cc1cc(C(C)(C)C)c(Cl)cc1-c1cc(=O)c(-c2cccc(C(N)=O)n2)c(C)[nH]1]

6-[6-(4-tert-butyl-5-chloro-2-methyl-phenyl)-2-
methyl-4-oxo-1H-pyridin-3-yl]pyridine-2-
carboxamide

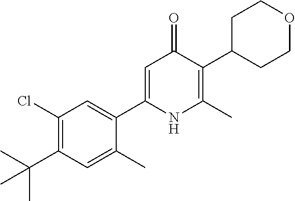 [SMILES: Cc1cc(C(C)(C)C)c(Cl)cc1-c1cc(=O)c(C2CCOCC2)c(C)[nH]1]

6-(4-tert-butyl-5-chloro-2-methyl-phenyl)-2-methyl-
3-tetrahydropyran-4-yl-1H-pyridin-4-one

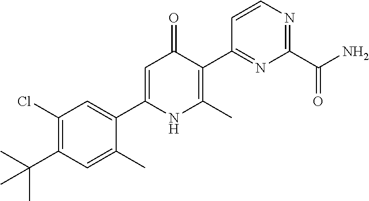 [SMILES: Cc1cc(C(C)(C)C)c(Cl)cc1-c1cc(=O)c(-c2ccnc(C(N)=O)n2)c(C)[nH]1]

4-[6-(4-tert-butyl-5-chloro-2-methyl-phenyl)-2-
methyl-4-oxo-1H-pyridin-3-yl]pyrimidine-2-
carboxamide

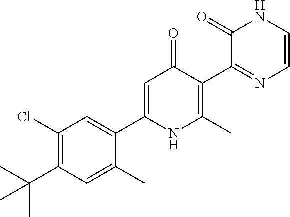 [SMILES: Cc1cc(C(C)(C)C)c(Cl)cc1-c1cc(=O)c(-c2ncc[nH]c2=O)c(C)[nH]1]

3-[6-(4-tert-butyl-5-chloro-2-methyl-phenyl)-2-
methyl-4-oxo-1H-pyridin-3-yl]-1H-pyrazin-2-one

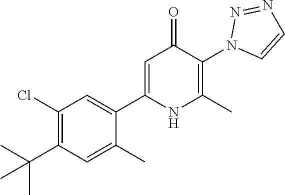 [SMILES: Cc1cc(C(C)(C)C)c(Cl)cc1-c1cc(=O)c(-n2ccnn2)c(C)[nH]1]

6-(4-tert-butyl-5-chloro-2-methyl-phenyl)-2-methyl-
3-(triazol-1-yl)-1H-pyridin-4-one

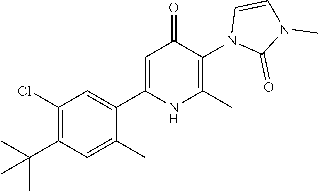 [SMILES: Cc1cc(C(C)(C)C)c(Cl)cc1-c1cc(=O)c(-n2ccn(C)c2=O)c(C)[nH]1]

6-(4-tert-butyl-5-chloro-2-methyl-phenyl)-2-methyl-
3-(3-methyl-2-oxo-imidazol-1-yl)-1H-pyridin-4-one

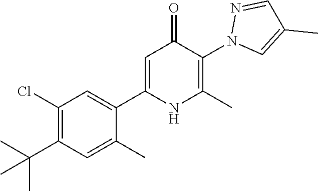 [SMILES: Cc1cnn(-c2c(C)[nH]c(-c3cc(Cl)c(C(C)(C)C)cc3C)cc2=O)c1]

6-(4-tert-butyl-5-chloro-2-methyl-phenyl)-2-methyl-
3-(4-methylpyrazol-1-yl)-1H-pyridin-4-one

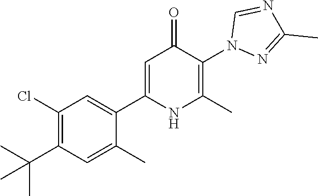 [SMILES: Cc1ncn(-c2c(C)[nH]c(-c3cc(Cl)c(C(C)(C)C)cc3C)cc2=O)n1]

6-(4-tert-butyl-5-chloro-2-methyl-phenyl)-2-methyl-
3-(3-methyl-1,2,4-triazol-1-yl)-1H-pyridin-4-one

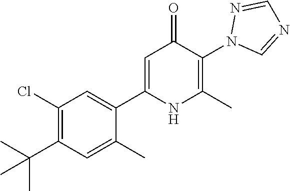 [SMILES: Cc1cc(C(C)(C)C)c(Cl)cc1-c1cc(=O)c(-n2cncn2)c(C)[nH]1]

6-(4-(tert-butyl)-5-chloro-2-methylphenyl)-2-
methyl-3-(1H-1,2,4-triazol-1-yl)pyridin-4(1H)-one

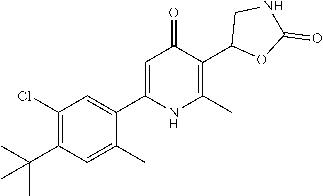 [SMILES: Cc1cc(C(C)(C)C)c(Cl)cc1-c1cc(=O)c(C2CNC(=O)O2)c(C)[nH]1]

5-(6-(4-(tert-butyl)-5-chloro-2-methylphenyl)-2-
methyl-4-oxo-1,4-dihydropyridin-3-yl)oxazolidin-2-
one

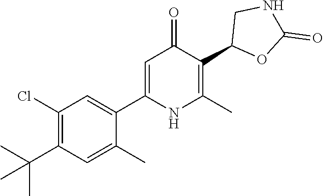 [SMILES: Cc1cc(C(C)(C)C)c(Cl)cc1-c1cc(=O)c([C@H]2CNC(=O)O2)c(C)[nH]1]

(S)-5-(6-(4-(tert-butyl)-5-chloro-2-methylphenyl)-2-
methyl-4-oxo-1,4-dihydropyridin-3-yl)oxazolidin-2-
one

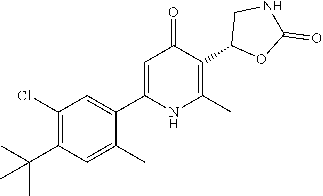 [SMILES: Cc1cc(C(C)(C)C)c(Cl)cc1-c1cc(=O)c([C@@H]2CNC(=O)O2)c(C)[nH]1]

(R)-5-(6-(4-(tert-butyl)-5-chloro-2-methylphenyl)-2-
methyl-4-oxo-1,4-dihydropyridin-3-yl)oxazolidin-2-
one

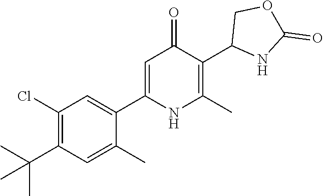 [SMILES: Cc1cc(C(C)(C)C)c(Cl)cc1-c1cc(=O)c(C2COC(=O)N2)c(C)[nH]1]

4-(6-(4-(tert-butyl)-5-chloro-2-methylphenyl)-2-
methyl-4-oxo-1,4-dihydropyridin-3-yl)oxazolidin-2-
one

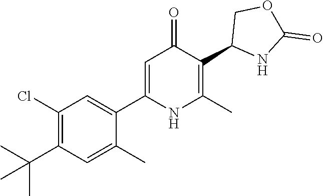 [SMILES: Cc1cc(C(C)(C)C)c(Cl)cc1-c1cc(=O)c([C@H]2COC(=O)N2)c(C)[nH]1]

(S)-4-(6-(4-(tert-butyl)-5-chloro-2-methylphenyl)-2-
methyl-4-oxo-1,4-dihydropyridin-3-yl)oxazolidin-2-
one

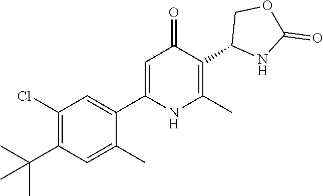 [SMILES: Cc1cc(C(C)(C)C)c(Cl)cc1-c1cc(=O)c([C@@H]2COC(=O)N2)c(C)[nH]1]

(R)-4-(6-(4-(tert-butyl)-5-chloro-2-methylphenyl)-2-
methyl-4-oxo-1,4-dihydropyridin-3-yl)oxazolidin-2-
one

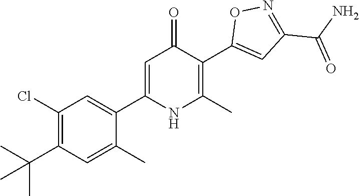 [SMILES: Cc1cc(C(C)(C)C)c(Cl)cc1-c1cc(=O)c(-c2cc(C(N)=O)no2)c(C)[nH]1]

5-[6-(4-tert-butyl-5-chloro-2-methyl-phenyl)-2-
methyl-4-oxo-1H-pyridin-3-yl]isoxazole-3-
carboxamide

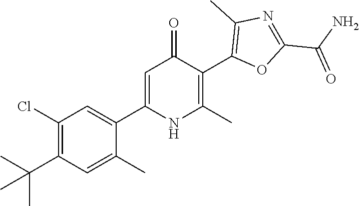 [SMILES: Cc1cc(C(C)(C)C)c(Cl)cc1-c1cc(=O)c(-c2oc(C(N)=O)nc2C)c(C)[nH]1]

5-[6-(4-tert-butyl-5-chloro-2-methyl-phenyl)-2-
methyl-4-oxo-1H-pyridin-3-yl]-4-methyl-oxazole-2-
carboxamide

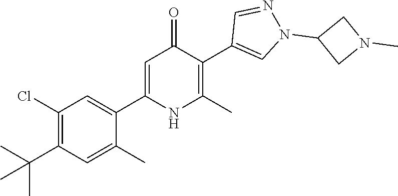 [SMILES: Cc1cc(C(C)(C)C)c(Cl)cc1-c1cc(=O)c(-c2cnn(C3CN(C)C3)c2)c(C)[nH]1]

6-(4-tert-butyl-5-chloro-2-methyl-phenyl)-2-methyl-
3-[1-(1-methylazetidin-3-yl)pyrazol-4-yl]-1H-
pyridin-4-one

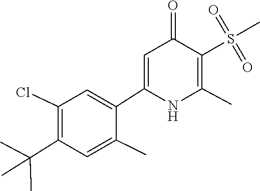 [SMILES: Cc1cc(C(C)(C)C)c(Cl)cc1-c1cc(=O)c(S(C)(=O)=O)c(C)[nH]1]

6-(4-tert-butyl-5-chloro-2-methyl-phenyl)-2-methyl-
3-methylsulfonyl-1H-pyridin-4-one

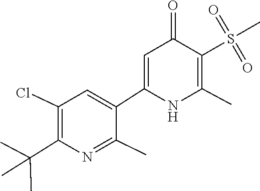 [SMILES: Cc1nc(C(C)(C)C)c(Cl)cc1-c1cc(=O)c(S(C)(=O)=O)c(C)[nH]1]

6′-(tert-butyl)-5′-chloro-2′,6-dimethyl-5-
(methylsulfonyl)-[2,3′-bipyridin]-4(1H)-one

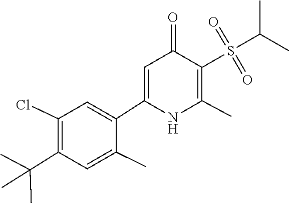 [SMILES: Cc1cc(C(C)(C)C)c(Cl)cc1-c1cc(=O)c(S(=O)(=O)C(C)C)c(C)[nH]1]

6-(4-tert-butyl-5-chloro-2-methyl-phenyl)-3-
isopropylsulfonyl-2-methyl-1H-pyridin-4-one

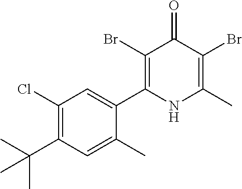 [SMILES: Cc1cc(C(C)(C)C)c(Cl)cc1-c1[nH]c(C)c(Br)c(=O)c1Br]

3,5-dibromo-2-(4-tert-butyl-5-chloro-2-methyl-
phenyl)-6-methyl-1H-pyridin-4-one

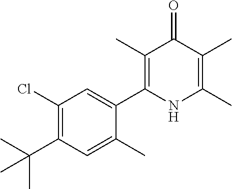 [SMILES: Cc1cc(C(C)(C)C)c(Cl)cc1-c1[nH]c(C)c(C)c(=O)c1C]

2-(4-tert-butyl-5-chloro-2-methyl-phenyl)-3,5,6-
trimethyl-1H-pyridin-4-one

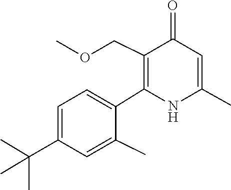 [SMILES: COCc1c(-c2ccc(C(C)(C)C)cc2C)[nH]c(C)cc1=O]

2-(4-tert-butyl-2-methyl-phenyl)-3-
(methoxymethyl)-6-methyl-1H-pyridin-4-one

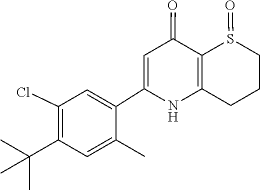 [SMILES: Cc1cc(C(C)(C)C)c(Cl)cc1-c1cc(=O)c2c([nH]1)CCCS2=O]

6-(4-tert-butyl-5-chloro-2-methyl-phenyl)-1-oxo-
2,3,4,5-tetrahydrothiopyrano[3,2-b]pyridin-8-one

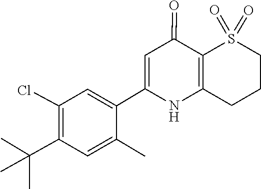 [SMILES: Cc1cc(C(C)(C)C)c(Cl)cc1-c1cc(=O)c2c([nH]1)CCCS2(=O)=O]

6-(4-tert-butyl-5-chloro-2-methyl-phenyl)-1,1-
dioxo-2,3,4,5-tetrahydrothiopyrano[3,2-b]pyridin-8-
one

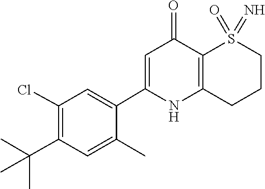 [SMILES: Cc1cc(C(C)(C)C)c(Cl)cc1-c1cc(=O)c2c([nH]1)CCCS2(=N)=O]

6-(4-tert-butyl-5-chloro-2-methyl-phenyl)-1-imino-
1-oxo-2,3,4,5-tetrahydrothiopyrano[3,2-b]pyridin-8-
one

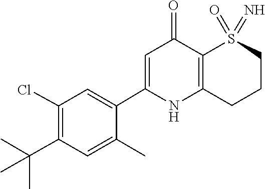 [SMILES: Cc1cc(C(C)(C)C)c(Cl)cc1-c1cc(=O)c2c([nH]1)CCC[S@]2(=N)=O]

(S)-6-(4-(tert-butyl)-5-chloro-2-methylphenyl)-1-
imino-1,3,4,5-tetrahydro-1l4-thiopyrano[3,2-
b]pyridin-8(2H)-one 1-oxide

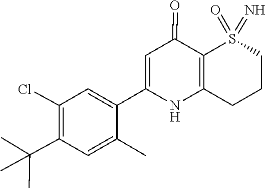 [SMILES: Cc1cc(C(C)(C)C)c(Cl)cc1-c1cc(=O)c2c([nH]1)CCC[S@@]2(=N)=O]

(R)-6-(4-(tert-butyl)-5-chloro-2-methylphenyl)-1-
imino-1,3,4,5-tetrahydro-1l4-thiopyrano[3,2-
b]pyridin-8(2H)-one 1-oxide

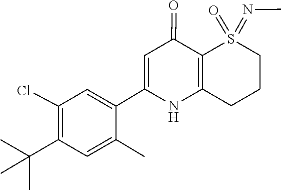 [SMILES: CN=S1(=O)CCCc2[nH]c(-c3cc(Cl)c(C(C)(C)C)cc3C)cc(=O)c21]

6-(4-(tert-butyl)-5-chloro-2-methylphenyl)-1-
(methylimino)-1,3,4,5-tetrahydro-1l4-
thiopyrano[3,2-b]pyridin-8(2H)-one 1-oxide

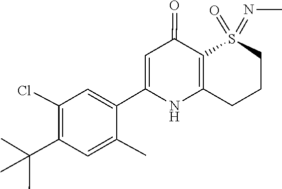 [SMILES: CN=[S@]1(=O)CCCc2[nH]c(-c3cc(Cl)c(C(C)(C)C)cc3C)cc(=O)c21]

(S)-6-(4-(tert-butyl)-5-chloro-2-methylphenyl)-1-
(methylimino)-1,3,4,5-tetrahydro-1l4-
thiopyrano[3,2-b]pyridin-8(2H)-one 1-oxide

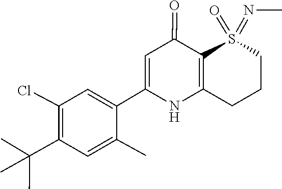 [SMILES: CN=[S@@]1(=O)CCCc2[nH]c(-c3cc(Cl)c(C(C)(C)C)cc3C)cc(=O)c21]

(R)-6-(4-(tert-butyl)-5-chloro-2-methylphenyl)-1-
(methylimino)-1,3,4,5-tetrahydro-1l4-
thiopyrano[3,2-b]pyridin-8(2H)-one 1-oxide

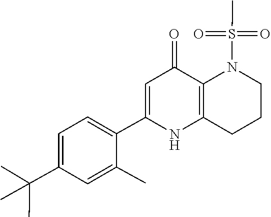 [SMILES: Cc1cc(C(C)(C)C)ccc1-c1cc(=O)c2c([nH]1)CCCN2S(C)(=O)=O]

2-(4-tert-butyl-2-methyl-phenyl)-5-methylsulfonyl-
1,6,7,8-tetrahydro-1,5-naphthyridin-4-one

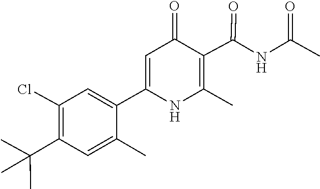 [SMILES: CC(=O)NC(=O)c1c(C)[nH]c(-c2cc(Cl)c(C(C)(C)C)cc2C)cc1=O]

N-acetyl-6-(4-(tert-butyl)-5-chloro-2-
methylphenyl)-2-methyl-4-oxo-1,4-dihydropyridine-
3-carboxamide

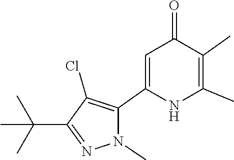 [SMILES: Cc1[nH]c(-c2c(Cl)c(C(C)(C)C)nn2C)cc(=O)c1C]

6-(3-(tert-butyl)-4-chloro-1-methyl-1H-pyrazol-5-
yl)-2,3-dimethylpyridin-4(1H)-one

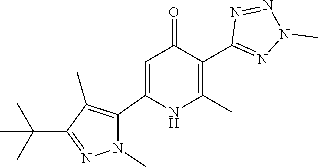 [SMILES: Cc1[nH]c(-c2c(C)c(C(C)(C)C)nn2C)cc(=O)c1-c1nnn(C)n1]

6-(3-(tert-butyl)-1,4-dimethyl-1H-pyrazol-5-yl)-2-
methyl-3-(2-methyl-2H-tetrazol-5-yl)pyridin-4(1H)-
one

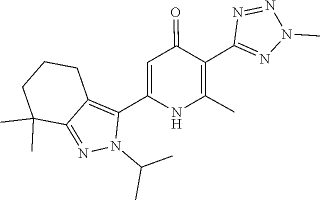

6-(2-isopropyl-7,7-dimethyl-4,5,6,7-tetrahydro-2H-
indazol-3-yl)-2-methyl-3-(2-methyl-2H-tetrazol-5-
yl)pyridin-4(1H)-one

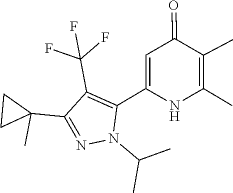 [SMILES: Cc1[nH]c(-c2c(C(F)(F)F)c(C3(C)CC3)nn2C(C)C)cc(=O)c1C]

6-(1-isopropyl-3-(1-methylcyclopropyl)-4-
(trifluoromethyl)-1H-pyrazol-5-yl)-2,3-
dimethylpyridin-4(1H)-one

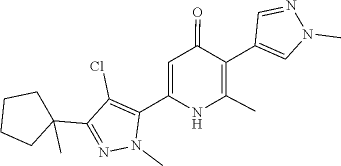 [SMILES: Cc1[nH]c(-c2c(Cl)c(C3(C)CCCC3)nn2C)cc(=O)c1-c1cnn(C)c1]

6-(4-chloro-1-methyl-3-(1-methylcyclopentyl)-1H-
pyrazol-5-yl)-2-methyl-3-(1-methyl-1H-pyrazol-4-
yl)pyridin-4(1H)-one

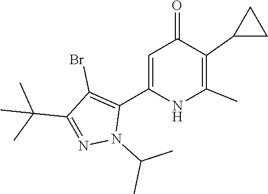 [SMILES: Cc1[nH]c(-c2c(Br)c(C(C)(C)C)nn2C(C)C)cc(=O)c1C1CC1]

6-(4-bromo-3-(tert-butyl)-1-isopropyl-1H-pyrazol-5-
yl)-3-cyclopropyl-2-methylpyridin-4(1H)-one

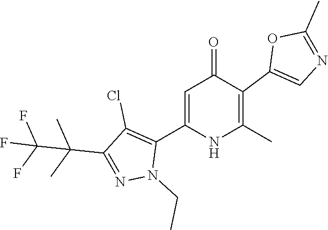 [SMILES: CCn1nc(C(C)(C)C(F)(F)F)c(Cl)c1-c1cc(=O)c(-c2cnc(C)o2)c(C)[nH]1]

6-(4-chloro-1-methyl-3-(1,1,1-trifluoro-2-
methylpropan-2-yl)-1H-pyrazol-5-yl)-2-methyl-3-
(2-methyloxazol-5-yl)pyridin-4(1H)-one

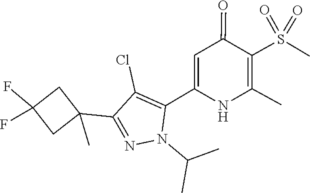 [SMILES: Cc1[nH]c(-c2c(Cl)c(C3(C)CC(F)(F)C3)nn2C(C)C)cc(=O)c1S(C)(=O)=O]

6-(4-chloro-3-(3,3-difluoro-1-methylcyclobutyl)-1-
isopropyl-1H-pyrazol-5-yl)-2-methyl-3-
(methylsulfonyl)pyridin-4(1H)-one

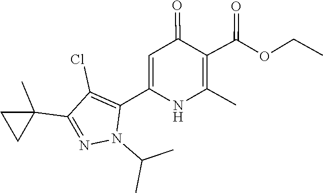 [SMILES: CCOC(=O)c1c(C)[nH]c(-c2c(Cl)c(C3(C)CC3)nn2C(C)C)cc1=O]

ethyl 6-(4-chloro-1-isopropyl-3-(1-
methylcyclopropyl)-1H-pyrazol-5-yl)-2-methyl-4-
oxo-1,4-dihydropyridine-3-carboxylate

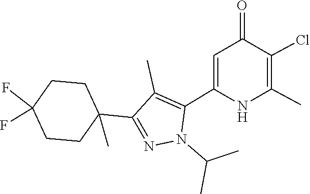 [SMILES: Cc1[nH]c(-c2c(C)c(C3(C)CCC(F)(F)CC3)nn2C(C)C)cc(=O)c1Cl]

3-chloro-6-(3-(4,4-difluoro-1-methylcyclohexyl)-1-
isopropyl-4-methyl-1H-pyrazol-5-yl)-2-
methylpyridin-4(1H)-one

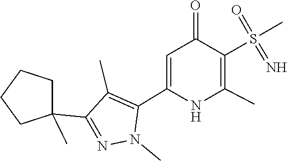 [SMILES: Cc1[nH]c(-c2c(C)c(C3(C)CCCC3)nn2C)cc(=O)c1S(C)(=N)=O]

6-(1,4-dimethyl-3-(1-methylcyclopentyl)-1H-
pyrazol-5-yl)-2-methyl-3-(S-
methylsulfonimidoyl)pyridin-4(1H)-one

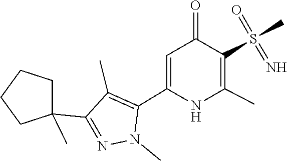 [SMILES: Cc1[nH]c(-c2c(C)c(C3(C)CCCC3)nn2C)cc(=O)c1[S@@](C)(=N)=O]

(S)-6-(1,4-dimethyl-3-(1-methylcyclopentyl)-1H-
pyrazol-5-yl)-2-methyl-3-(S-
methylsulfonimidoyl)pyridin-4(1H)-one

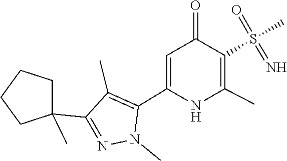 [SMILES: Cc1[nH]c(-c2c(C)c(C3(C)CCCC3)nn2C)cc(=O)c1[S@](C)(=N)=O]

(R)-6-(1,4-dimethyl-3-(1-methylcyclopentyl)-1H-
pyrazol-5-yl)-2-methyl-3-(S-
methylsulfonimidoyl)pyridin-4(1H)-one

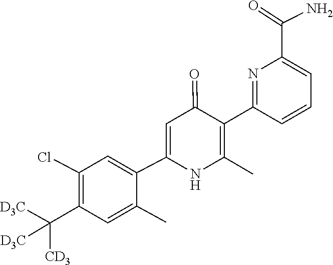 [SMILES: [2H]C([2H])([2H])C(c1cc(C)c(-c2cc(=O)c(-c3cccc(C(N)=O)n3)c(C)[nH]2)cc1Cl)(C([2H])([2H])[2H])C([2H])([2H])[2H]]

6′-(5-chloro-2-methyl-4-(2-(methyl-d3)propan-2-yl-
1,1,1,3,3,3-d6)phenyl)-2′-methyl-4′-oxo-1′,4′-
dihydro-[2,3′-bipyridine]-6-carboxamide

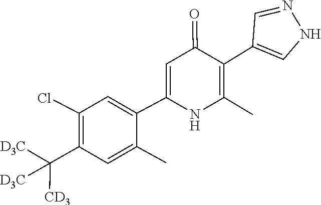 [SMILES: [2H]C([2H])([2H])C(c1cc(C)c(-c2cc(=O)c(-c3cn[nH]c3)c(C)[nH]2)cc1Cl)(C([2H])([2H])[2H])C([2H])([2H])[2H]]

6-(5-chloro-2-methyl-4-(2-(methyl-d3)propan-2-yl-
1,1,1,3,3,3-d6)phenyl)-2-methyl-3-(1H-pyrazol-4-
yl)pyridin-4(1H)-one

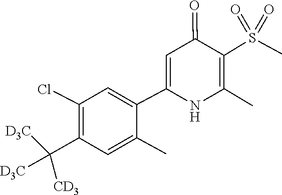 [SMILES: [2H]C([2H])([2H])C(c1cc(C)c(-c2cc(=O)c(S(C)(=O)=O)c(C)[nH]2)cc1Cl)(C([2H])([2H])[2H])C([2H])([2H])[2H]]

6-(5-chloro-2-methyl-4-(2-(methyl-d3)propan-2-yl-
1,1,1,3,3,3-d6)phenyl)-2-methyl-3-
(methylsulfonyl)pyridin-4(1H)-one

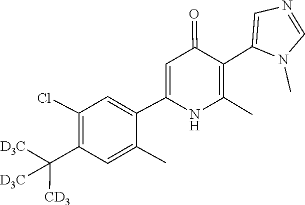 [SMILES: [2H]C([2H])([2H])C(c1cc(C)c(-c2cc(=O)c(-c3cncn3C)c(C)[nH]2)cc1Cl)(C([2H])([2H])[2H])C([2H])([2H])[2H]]

6-(5-chloro-2-methyl-4-(2-(methyl-d3)propan-2-yl-
1,1,1,3,3,3-d6)phenyl)-2-methyl-3-(1-methyl-1H-
imidazol-5-yl)pyridin-4(1H)-one

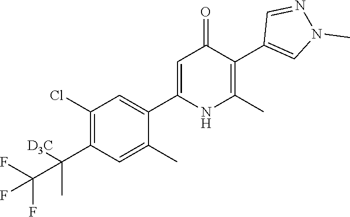 [SMILES: [2H]C([2H])([2H])C(C)(c1cc(C)c(-c2cc(=O)c(-c3cnn(C)c3)c(C)[nH]2)cc1Cl)C(F)(F)F]

6-(5-chloro-2-methyl-4-(1,1,1-trifluoro-2-
methylpropan-2-yl-3,3,3-d3)phenyl)-2-methyl-3-(1-
methyl-1H-pyrazol-4-yl)pyridin-4(1H)-one

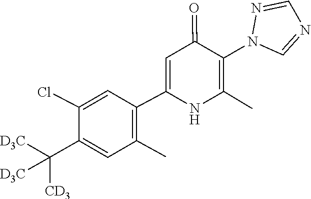 [SMILES: [2H]C([2H])([2H])C(c1cc(C)c(-c2cc(=O)c(-n3cncn3)c(C)[nH]2)cc1Cl)(C([2H])([2H])[2H])C([2H])([2H])[2H]]

6-(5-chloro-2-methyl-4-(2-methyl-d3)propan-2-yl-
1,1,1,3,3,3-d6)phenyl)-2-methyl-3-(1H-1,2,4-triazol-
1-yl)pyridin-4(1H)-one

In some embodiments, the invention relates to a compound selected from:

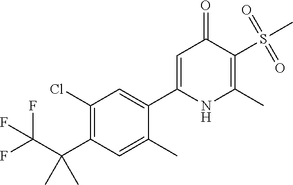 [SMILES: Cc1cc(C(C)(C)C(F)(F)F)c(Cl)cc1-c1cc(=O)c(S(C)(=O)=O)c(C)[nH]1]

6-[5-chloro-2-methyl-4-(2,2,2-trifluoro-1,1-dimethyl-ethyl)phenyl]-2-methyl-3-methylsulfonyl-1H-pyridin-4-one;

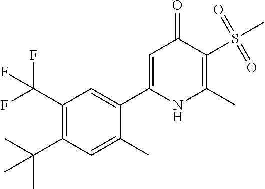 [SMILES: Cc1cc(C(C)(C)C)c(C(F)(F)F)cc1-c1cc(=O)c(S(C)(=O)=O)c(C)[nH]1]

6-[4-tert-butyl-2-methyl-5-(trifluoromethyl)phenyl]-2-methyl-3-methylsulfonyl-1H-pyridin-4-one;

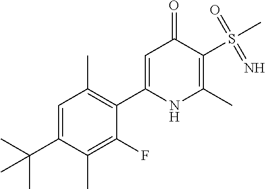 [SMILES: Cc1cc(C(C)(C)C)c(C)c(F)c1-c1cc(=O)c(S(C)(=N)=O)c(C)[nH]1]

6-(4-tert-butyl-2-fluoro-3,6-dimethyl-phenyl)-2-methyl-3-(methylsulfonimidoyl)-1H-pyridin-4-one;

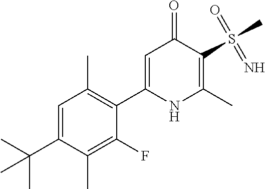 [SMILES: Cc1cc(C(C)(C)C)c(C)c(F)c1-c1cc(=O)c([S@@](C)(=N)=O)c(C)[nH]1]

(S)-6-(4-tert-butyl-2-fluoro-3,6-dimethyl-phenyl)-2-methyl-3-(methylsulfonimidoyl)-1H-pyridin-4-one;

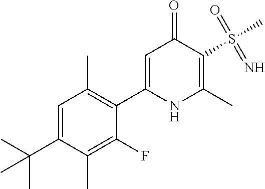 [SMILES: Cc1cc(C(C)(C)C)c(C)c(F)c1-c1cc(=O)c([S@](C)(=N)=O)c(C)[nH]1]

(R)-6-(4-tert-butyl-2-fluoro-3,6-dimethyl-phenyl)-2-methyl-3-(methylsulfonimidoyl)-1H-pyridin-4-one;

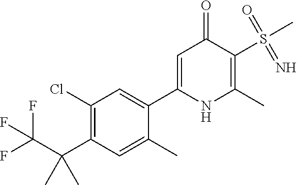 [SMILES: Cc1cc(C(C)(C)C(F)(F)F)c(Cl)cc1-c1cc(=O)c(S(C)(=N)=O)c(C)[nH]1]

6-[5-chloro-2-methyl-4-(2,2,2-trifluoro-1,1-dimethyl-ethyl)phenyl]-2-methyl-3-(methylsulfonimidoyl)-1H-pyridin-4-one;

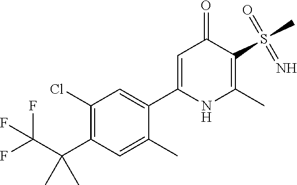 [SMILES: Cc1cc(C(C)(C)C(F)(F)F)c(Cl)cc1-c1cc(=O)c([S@@](C)(=N)=O)c(C)[nH]1]

(S)-6-[5-chloro-2-methyl-4-(2,2,2-trifluoro-1,1-dimethyl-ethyl)phenyl]-2-methyl-3-(methylsulfonimidoyl)-1H-pyridin-4-one;

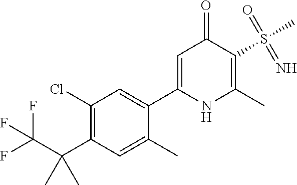 [SMILES: Cc1cc(C(C)(C)C(F)(F)F)c(Cl)cc1-c1cc(=O)c([S@](C)(=N)=O)c(C)[nH]1]

(R)-6-[5-chloro-2-methyl-4-(2,2,2-trifluoro-1,1-dimethyl-ethyl)phenyl]-2-methyl-3-(methylsulfonimidoyl)-1H-pyridin-4-one;

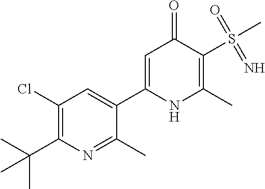 [SMILES: Cc1nc(C(C)(C)C)c(Cl)cc1-c1cc(=O)c(S(C)(=N)=O)c(C)[nH]1]

6-(6-tert-butyl-5-chloro-2-methyl-3-pyridyl)-2-methyl-3-(methylsulfonimidoyl)-1H-pyridin-4-one;

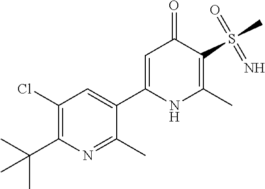 [SMILES: Cc1nc(C(C)(C)C)c(Cl)cc1-c1cc(=O)c([S@@](C)(=N)=O)c(C)[nH]1]

(S)-6-(6-tert-butyl-5-chloro-2-methyl-3-pyridyl)-2-methyl-3-(methylsulfonimidoyl)-1H-pyridin-4-one;

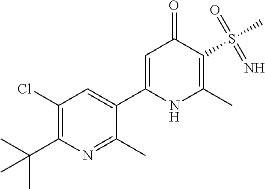 [SMILES: Cc1nc(C(C)(C)C)c(Cl)cc1-c1cc(=O)c([S@](C)(=N)=O)c(C)[nH]1]

(R)-6-(6-tert-butyl-5-chloro-2-methyl-3-pyridyl)-2-methyl-3-(methylsulfonimidoyl)-1H-pyridin-4-one;

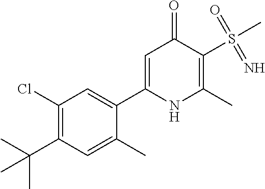 [SMILES: Cc1cc(C(C)(C)C)c(Cl)cc1-c1cc(=O)c(S(C)(=N)=O)c(C)[nH]1]

6-(4-tert-butyl-5-chloro-2-methyl-phenyl)-2-methyl-3-(methylsulfonimidoyl)-1H-pyridin-4-one;

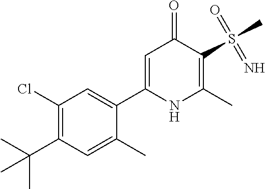 [SMILES: Cc1cc(C(C)(C)C)c(Cl)cc1-c1cc(=O)c([S@@](C)(=N)=O)c(C)[nH]1]

(S)-6-(4-tert-butyl-5-chloro-2-methyl-phenyl)-2-methyl-3-(methylsulfonimidoyl)-1H-pyridin-4-one;

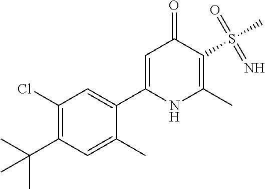 [SMILES: Cc1cc(C(C)(C)C)c(Cl)cc1-c1cc(=O)c([S@](C)(=N)=O)c(C)[nH]1]

(R)-6-(4-tert-butyl-5-chloro-2-methyl-phenyl)-2-methyl-3-(methylsulfonimidoyl)-1H-pyridin-4-one;

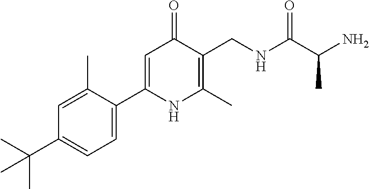 [SMILES: Cc1cc(C(C)(C)C)ccc1-c1cc(=O)c(CNC(=O)[C@H](C)N)c(C)[nH]1]

(2S)-2-amino-N-[[6-(4-tert-butyl-2-methyl-phenyl)-2-methyl-4-oxo-1H-pyridin-3-yl]methyl]propanamide;

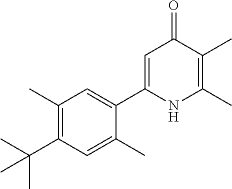 [SMILES: Cc1cc(C(C)(C)C)c(C)cc1-c1cc(=O)c(C)c(C)[nH]1]

6-(4-tert-butyl-2,5-dimethyl-phenyl)-2,3-dimethyl-1H-pyridin-4-one;

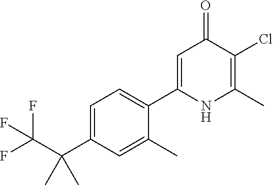 [SMILES: Cc1cc(C(C)(C)C(F)(F)F)ccc1-c1cc(=O)c(Cl)c(C)[nH]1]

3-chloro-2-methyl-6-[2-methyl-4-(2,2,2-trifluoro-1,1-dimethyl-ethyl)phenyl]-1H-pyridin-4-one;

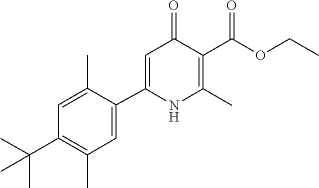 [SMILES: CCOC(=O)c1c(C)[nH]c(-c2cc(C)c(C(C)(C)C)cc2C)cc1=O]

ethyl 6-(4-tert-butyl-2,5-dimethyl-phenyl)-2-methyl-4-oxo-1H-pyridine-3-carboxylate;

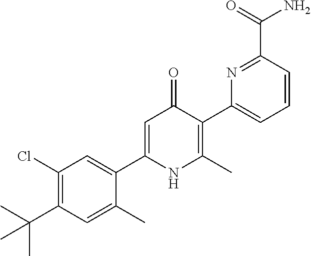 [SMILES: Cc1cc(C(C)(C)C)c(Cl)cc1-c1cc(=O)c(-c2cccc(C(N)=O)n2)c(C)[nH]1]

6-[6-(4-tert-butyl-5-chloro-2-methyl-phenyl)-2-methyl-4-oxo-1H-pyridin-3-yl]pyridine-2-carboxamide;

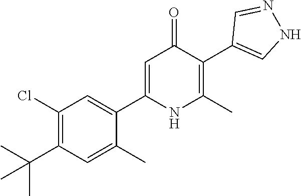 [SMILES: Cc1cc(C(C)(C)C)c(Cl)cc1-c1cc(=O)c(-c2cn[nH]c2)c(C)[nH]1]

6-(4-tert-butyl-5-chloro-2-methyl-phenyl)-2-methyl-3-(1H-pyrazol-4-yl)-1H-pyridin-4-one;

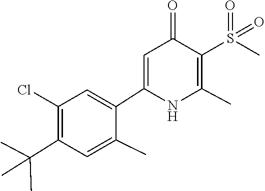 [SMILES: Cc1cc(C(C)(C)C)c(Cl)cc1-c1cc(=O)c(S(C)(=O)=O)c(C)[nH]1]

6-(4-tert-butyl-5-chloro-2-methyl-phenyl)-2-methyl-3-methylsulfonyl-1H-pyridin-4-one;

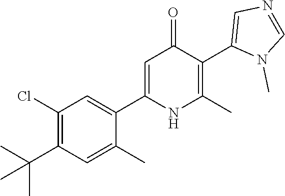 [SMILES: Cc1cc(C(C)(C)C)c(Cl)cc1-c1cc(=O)c(-c2cncn2C)c(C)[nH]1]

6-(4-(tert-butyl)-5-chloro-2-methylphenyl)-2-methyl-3-(1-methyl-1H-imidazol-5-yl)pyridin-4(1H)-one;

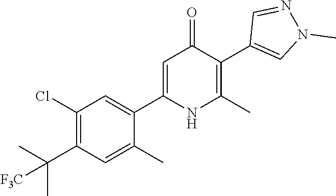 [SMILES: Cc1cc(C(C)(C)C(F)(F)F)c(Cl)cc1-c1cc(=O)c(-c2cnn(C)c2)c(C)[nH]1]

6-(5-chloro-2-methyl-4-(1,1,1-trifluoro-2-methylpropan-2-yl)phenyl)-2-methyl-3-(1-methyl-1H-pyrazol-4-yl)pyridin-4(1H)-one; and

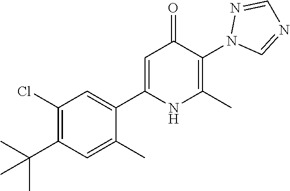 [SMILES: Cc1cc(C(C)(C)C)c(Cl)cc1-c1cc(=O)c(-n2cncn2)c(C)[nH]1]

6-(4-(tert-butyl)-5-chloro-2-methylphenyl)-2-methyl-3-(1H-1,2,4-triazol-1-yl)pyridin-4(1H)-one, or a pharmaceutically acceptable salt thereof.

In some embodiments, the invention relates to a compound of formula

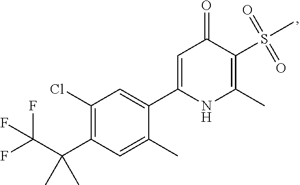 [SMILES: Cc1cc(C(C)(C)C(F)(F)F)c(Cl)cc1-c1cc(=O)c(S(C)(=O)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-[5-chloro-2-methyl-4-(2,2,2-trifluoro-1,1-dimethyl-ethyl)phenyl]-2-methyl-3-methylsulfonyl-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

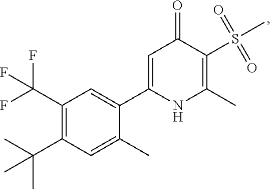 [SMILES: Cc1cc(C(C)(C)C)c(C(F)(F)F)cc1-c1cc(=O)c(S(C)(=O)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-[4-tert-butyl-2-methyl-5-(trifluoromethyl)phenyl]-2-methyl-3-methylsulfonyl-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

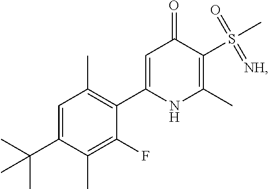 [SMILES: Cc1cc(C(C)(C)C)c(C)c(F)c1-c1cc(=O)c(S(C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-(4-tert-butyl-2-fluoro-3,6-dimethyl-phenyl)-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to the first eluting isomer of the compound 6-(4-tert-butyl-2-fluoro-3,6-dimethyl-phenyl)-2-methyl-3-(methylsulfonimidoyl)-1H-pyridin-4-one, or a pharmaceutically acceptable salt thereof, when the stereoisomers are resolved using an appropriate separation technique. In other embodiments, the invention relates to the foregoing compound in non-salt form. Such compound is considered to be a “compound of the invention,” as that term is used herein.

In some embodiments, the invention relates to the second eluting isomer of the compound 6-(4-tert-butyl-2-fluoro-3,6-dimethyl-phenyl)-2-methyl-3-(methylsulfonimidoyl)-1H-pyridin-4-one, or a pharmaceutically acceptable salt thereof, when the stereoisomers are resolved using an appropriate separation technique. In other embodiments, the invention relates to the foregoing compound in non-salt form. Such compound is considered to be a “compound of the invention,” as that term is used herein.

In some embodiments, the invention relates to a compound of formula

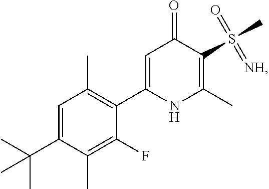 [SMILES: Cc1cc(C(C)(C)C)c(C)c(F)c1-c1cc(=O)c([S@@](C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (S)-6-(4-tert-butyl-2-fluoro-3,6-dimethyl-phenyl)-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

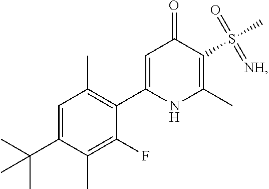 [SMILES: Cc1cc(C(C)(C)C)c(C)c(F)c1-c1cc(=O)c([S@](C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (R)-6-(4-tert-butyl-2-fluoro-3,6-dimethyl-phenyl)-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

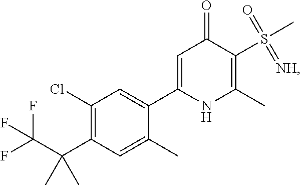 [SMILES: Cc1cc(C(C)(C)C(F)(F)F)c(Cl)cc1-c1cc(=O)c(S(C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-[5-chloro-2-methyl-4-(2,2,2-trifluoro-1,1-dimethyl-ethyl)phenyl]-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to the first eluting isomer of the compound 6-[5-chloro-2-methyl-4-(2,2,2-trifluoro-1,1-dimethyl-ethyl)phenyl]-2-methyl-3-(methylsulfonimidoyl)-1H-pyridin-4-one, or a pharmaceutically acceptable salt thereof, when the stereoisomers are resolved using an appropriate separation technique. In other embodiments, the invention relates to the foregoing compound in non-salt form. Such compound is considered to be a “compound of the invention,” as that term is used herein.

In some embodiments, the invention relates to the second eluting isomer of the compound 6-[5-chloro-2-methyl-4-(2,2,2-trifluoro-1,1-dimethyl-ethyl)phenyl]-2-methyl-3-(methylsulfonimidoyl)-1H-pyridin-4-one, or a pharmaceutically acceptable salt thereof, when the stereoisomers are resolved using an appropriate separation technique. In other embodiments, the invention relates to the foregoing compound in non-salt form. Such compound is considered to be a “compound of the invention,” as that term is used herein.

In some embodiments, the invention relates to a compound of formula

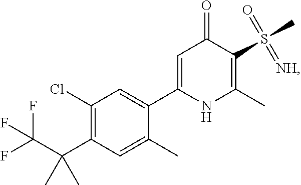 [SMILES: Cc1cc(C(C)(C)C(F)(F)F)c(Cl)cc1-c1cc(=O)c([S@@](C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (S)-6-[5-chloro-2-methyl-4-(2,2,2-trifluoro-1,1-dimethyl-ethyl)phenyl]-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

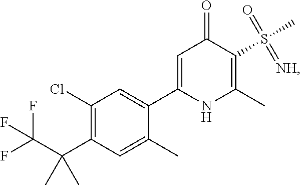 [SMILES: Cc1cc(C(C)(C)C(F)(F)F)c(Cl)cc1-c1cc(=O)c([S@](C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (R)-6-[5-chloro-2-methyl-4-(2,2,2-trifluoro-1,1-dimethyl-ethyl)phenyl]-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

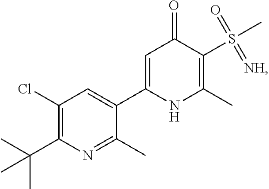 [SMILES: Cc1nc(C(C)(C)C)c(Cl)cc1-c1cc(=O)c(S(C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-(6-tert-butyl-5-chloro-2-methyl-3-pyridyl)-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to the first eluting isomer of the compound 6-(6-tert-butyl-5-chloro-2-methyl-3-pyridyl)-2-methyl-3-(methylsulfonimidoyl)-1H-pyridin-4-one, or a pharmaceutically acceptable salt thereof, when the stereoisomers are resolved using an appropriate separation technique. In other embodiments, the invention relates to the foregoing compound in non-salt form. Such compound is considered to be a “compound of the invention,” as that term is used herein.

In some embodiments, the invention relates to the second eluting isomer of the compound 6-(6-tert-butyl-5-chloro-2-methyl-3-pyridyl)-2-methyl-3-(methylsulfonimidoyl)-1H-pyridin-4-one, or a pharmaceutically acceptable salt thereof, when the stereoisomers are resolved using an appropriate separation technique. In other embodiments, the invention relates to the foregoing compound in non-salt form. Such compound is considered to be a “compound of the invention,” as that term is used herein.

In some embodiments, the invention relates to a compound of formula

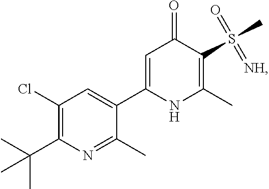 [SMILES: Cc1nc(C(C)(C)C)c(Cl)cc1-c1cc(=O)c([S@@](C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (S)-6-(6-tert-butyl-5-chloro-2-methyl-3-pyridyl)-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

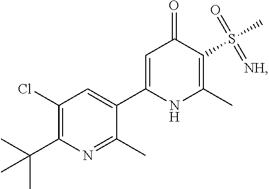 [SMILES: Cc1nc(C(C)(C)C)c(Cl)cc1-c1cc(=O)c([S@](C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (R)-6-(6-tert-butyl-5-chloro-2-methyl-3-pyridyl)-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

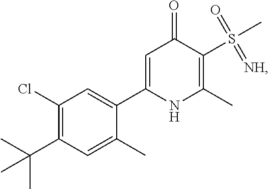 [SMILES: Cc1cc(C(C)(C)C)c(Cl)cc1-c1cc(=O)c(S(C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-(4-tert-butyl-5-chloro-2-methyl-phenyl)-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to the first eluting isomer of the compound 6-(4-tert-butyl-5-chloro-2-methyl-phenyl)-2-methyl-3-(methylsulfonimidoyl)-1H-pyridin-4-one, or a pharmaceutically acceptable salt thereof, when the stereoisomers are resolved using an appropriate separation technique. In other embodiments, the invention relates to the foregoing compound in non-salt form. Such compound is considered to be a “compound of the invention,” as that term is used herein.

In some embodiments, the invention relates to the second eluting isomer of the compound 6-(4-tert-butyl-5-chloro-2-methyl-phenyl)-2-methyl-3-(methylsulfonimidoyl)-1H-pyridin-4-one, or a pharmaceutically acceptable salt thereof, when the stereoisomers are resolved using an appropriate separation technique. In other embodiments, the invention relates to the foregoing compound in non-salt form. Such compound is considered to be a “compound of the invention,” as that term is used herein.

In some embodiments, the invention relates to a compound of formula

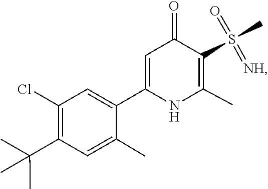 [SMILES: Cc1cc(C(C)(C)C)c(Cl)cc1-c1cc(=O)c([S@@](C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (S)-6-(4-tert-butyl-5-chloro-2-methyl-phenyl)-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

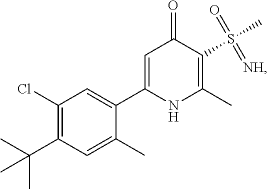 [SMILES: Cc1cc(C(C)(C)C)c(Cl)cc1-c1cc(=O)c([S@](C)(=N)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (R)-6-(4-tert-butyl-5-chloro-2-methyl-phenyl)-2-methyl-3-(methylsulfonimidoyl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

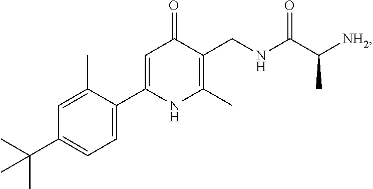 [SMILES: Cc1cc(C(C)(C)C)ccc1-c1cc(=O)c(CNC(=O)[C@H](C)N)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (2S)-2-amino-N-[[6-(4-tert-butyl-2-methyl-phenyl)-2-methyl-4-oxo-1H-pyridin-3-yl]methyl]propanamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

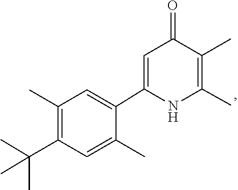 [SMILES: Cc1cc(C(C)(C)C)c(C)cc1-c1cc(=O)c(C)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-(4-tert-butyl-2,5-dimethyl-phenyl)-2,3-dimethyl-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

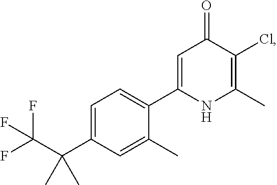 [SMILES: Cc1cc(C(C)(C)C(F)(F)F)ccc1-c1cc(=O)c(Cl)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 3-chloro-2-methyl-6-[2-methyl-4-(2,2,2-trifluoro-1,1-dimethyl-ethyl)phenyl]-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

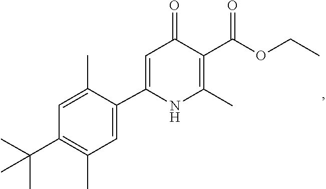 [SMILES: CCOC(=O)c1c(C)[nH]c(-c2cc(C)c(C(C)(C)C)cc2C)cc1=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound ethyl 6-(4-tert-butyl-2,5-dimethyl-phenyl)-2-methyl-4-oxo-1H-pyridine-3-carboxylate, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

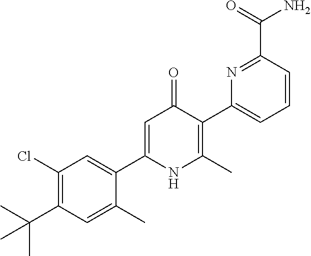 [SMILES: Cc1cc(C(C)(C)C)c(Cl)cc1-c1cc(=O)c(-c2cccc(C(N)=O)n2)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound ethy6-[6-(4-tert-butyl-5-chloro-2-methyl-phenyl)-2-methyl-4-oxo-1H-pyridin-3-yl]pyridine-2-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

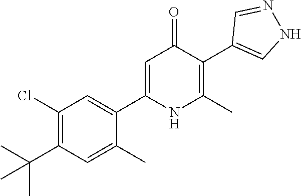 [SMILES: Cc1cc(C(C)(C)C)c(Cl)cc1-c1cc(=O)c(-c2cn[nH]c2)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-(4-tert-butyl-5-chloro-2-methyl-phenyl)-2-methyl-3-(1H-pyrazol-4-yl)-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

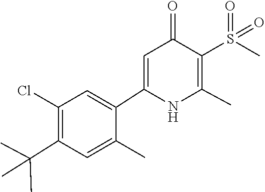 [SMILES: Cc1cc(C(C)(C)C)c(Cl)cc1-c1cc(=O)c(S(C)(=O)=O)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-(4-tert-butyl-5-chloro-2-methyl-phenyl)-2-methyl-3-methylsulfonyl-1H-pyridin-4-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

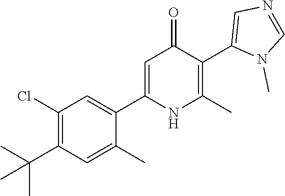 [SMILES: Cc1cc(C(C)(C)C)c(Cl)cc1-c1cc(=O)c(-c2cncn2C)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-(4-(tert-butyl)-5-chloro-2-methylphenyl)-2-methyl-3-(1-methyl-1H-imidazol-5-yl)pyridin-4(1H)-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

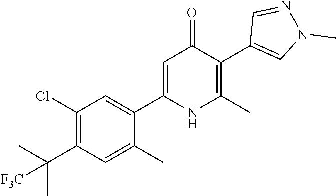 [SMILES: Cc1cc(C(C)(C)C(F)(F)F)c(Cl)cc1-c1cc(=O)c(-c2cnn(C)c2)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-(5-chloro-2-methyl-4-(1,1,1-trifluoro-2-methylpropan-2-yl)phenyl)-2-methyl-3-(1-methyl-1H-pyrazol-4-yl)pyridin-4(1H)-one, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

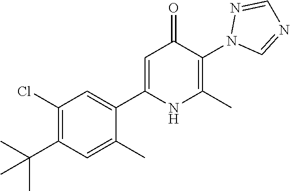 [SMILES: Cc1cc(C(C)(C)C)c(Cl)cc1-c1cc(=O)c(-n2cncn2)c(C)[nH]1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 6-(4-(tert-butyl)-5-chloro-2-methylphenyl)-2-methyl-3-(1H-1,2,4-triazol-1-yl)pyridin-4(1H)-one, or a pharmaceutically acceptable salt thereof. In other embodiments, the invention relates to the foregoing compound in non-salt form. Such compound is considered to be a “compound of the invention,” as that term is used herein.

### Salts, Compositions, Uses, Formulation, Administration and Additional Agents

### Pharmaceutically Acceptable Salts and Compositions

As discussed herein, the invention provides compounds, and pharmaceutically acceptable salts thereof, that are inhibitors of voltage-gated sodium channels, and thus the present compounds, and pharmaceutically acceptable salts thereof, are useful for the treatment of diseases, disorders, and conditions including, but not limited to chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia. Accordingly, in another aspect of the invention, pharmaceutical compositions are provided, wherein these compositions comprise a compound as described herein, or a pharmaceutically acceptable salt thereof, and optionally comprise a pharmaceutically acceptable carrier, adjuvant or vehicle. In certain embodiments, these compositions optionally further comprise one or more additional therapeutic agents. In some embodiments, the additional therapeutic agent is a sodium channel inhibitor.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” of a compound of this invention includes any non-toxic salt that, upon administration to a recipient, is capable of providing, either directly or indirectly, a compound of this invention or an inhibitorily active metabolite or residue thereof. The salt may be in pure form, in a mixture (e.g., solution, suspension, or colloid) with one or more other substances, or in the form of a hydrate, solvate, or co-crystal. As used herein, the term “inhibitorily active metabolite or residue thereof” means that a metabolite or residue thereof is also an inhibitor of a voltage-gated sodium channel.

Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Pharmaceutically acceptable salts of the compound of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As described herein, the pharmaceutically acceptable compositions of the invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In another aspect, the invention features a pharmaceutical composition comprising a compound of the invention, or a pharmaceutically acceptable salt thereof, and a pharmaceutically acceptable carrier.

In another aspect, the invention features a pharmaceutical composition comprising a therapeutically effective amount of a compound, or a pharmaceutically acceptable salt thereof, and one or more pharmaceutically acceptable carriers or vehicles.

### Uses of Compounds and Pharmaceutically Acceptable Salts and Compositions

In another aspect, the invention features a method of inhibiting a voltage-gated sodium channel in a subject comprising administering to the subject a compound of the invention or a pharmaceutically acceptable salt thereof or a pharmaceutical composition thereof. In another aspect, the voltage-gated sodium channel is Na_{V}1.8.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of gut pain, wherein gut pain comprises inflammatory bowel disease pain, Crohn's disease pain, irritable bowel syndrome, endometriosis, polycyctic ovarian disease, salpingitis, cervicitis or interstitial cystitis pain wherein said method comprises administering an effective amount of a compound of the invention, a pharmaceutically acceptable salt thereof or a pharmaceutical composition thereof.

In yet another aspect, the invention features a method of treating or lessening the severity in a subject of neuropathic pain comprising administering an effective amount of a compound of the invention, a pharmaceutically acceptable salt thereof or a pharmaceutical composition thereof. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In yet another aspect, the invention features a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia wherein said method comprises administering an effective amount of a compound of the invention, a pharmaceutically acceptable salt thereof or a pharmaceutical composition thereof.

In yet another aspect, the invention features a method of treating or lessening the severity in a subject of musculoskeletal pain comprising administering an effective amount of a compound of the invention, a pharmaceutically acceptable salt thereof or a pharmaceutical composition thereof. In some aspects, the musculoskeletal pain comprises osteoarthritis pain.

In yet another aspect, the invention features a method of treating or lessening the severity in a subject of musculoskeletal pain, wherein musculoskeletal pain comprises osteoarthritis pain, back pain, cold pain, burn pain or dental pain wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain, ankylosing spondylitis or vulvodynia wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises fibromyalgia pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises reflex sympathetic dystrophy pain, wherein said method comprises administering an effective amount of a compound of the invention, a pharmaceutically acceptable salt thereof or a pharmaceutical composition thereof.

In yet another aspect, the invention features a method of treating or lessening the severity in a subject of pathological cough wherein said method comprises administering an effective amount of a compound of the invention, a pharmaceutically acceptable salt thereof or a pharmaceutical composition thereof.

In yet another aspect, the invention features a method of treating or lessening the severity in a subject of acute pain comprising administering an effective amount of a compound of the invention, a pharmaceutically acceptable salt thereof or a pharmaceutical composition thereof. In some aspects, the acute pain comprises acute post-operative pain.

In yet another aspect, the invention features a method of treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain) comprising administering an effective amount of a compound of the invention, a pharmaceutically acceptable salt thereof or a pharmaceutical composition thereof.

In yet another aspect, the invention features a method of treating or lessening the severity in a subject of bunionectomy pain comprising administering an effective amount of a compound of the invention, a pharmaceutically acceptable salt thereof or a pharmaceutical composition thereof.

In yet another aspect, the invention features a method of treating or lessening the severity in a subject of shoulder arthroplasty pain or shoulder arthroscopy pain comprising administering an effective amount of a compound of the invention, a pharmaceutically acceptable salt thereof or a pharmaceutical composition thereof.

In yet another aspect, the invention features a method of treating or lessening the severity in a subject of herniorrhaphy pain comprising administering an effective amount of a compound of the invention, a pharmaceutically acceptable salt thereof or a pharmaceutical composition thereof.

In yet another aspect, the invention features a method of treating or lessening the severity in a subject of abdominoplasty pain comprising administering an effective amount of a compound of the invention, a pharmaceutically acceptable salt thereof or a pharmaceutical composition thereof.

In yet another aspect, the invention features a method of treating or lessening the severity in a subject of visceral pain comprising administering an effective amount of a compound of the invention, a pharmaceutically acceptable salt thereof or a pharmaceutical composition thereof. In some aspects, the visceral pain comprises visceral pain from abdominoplasty.

In yet another aspect, the invention features a method of treating or lessening the severity in a subject of a neurodegenerative disease comprising administering an effective amount of a compound of the invention, a pharmaceutically acceptable salt thereof or a pharmaceutical composition thereof. In some aspects, the neurodegenerative disease comprises multiple sclerosis. In some aspects, the neurodegenerative disease comprises Pitt Hopkins Syndrome (PTHS).

In yet another aspect, the invention features a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a method of treating or lessening the severity in a subject of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia rheumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodiscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarthropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility, comprising administering an effective amount of a compound of the invention, a pharmaceutically acceptable salt thereof or a pharmaceutical composition thereof.

In another aspect, the invention features a method of treating or lessening the severity in a subject of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain, comprising administering an effective amount of a compound of the invention, a pharmaceutically acceptable salt thereof or a pharmaceutical composition thereof.

In another aspect, the invention features a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain, comprising administering an effective amount of a compound of the invention, a pharmaceutically acceptable salt thereof or a pharmaceutical composition thereof.

### Compounds, Pharmaceutically Acceptable Salts, and Compositions for Use

In another aspect, the invention features a compound of the invention, or a pharmaceutically acceptable salt or pharmaceutical composition thereof, for use as a medicament.

In another aspect, the invention features a compound of the invention, or a pharmaceutically acceptable salt or pharmaceutical composition thereof, for use in a method of inhibiting a voltage-gated sodium channel in a subject. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In another aspect, the invention features a compound of the invention, or a pharmaceutically acceptable salt or pharmaceutical composition thereof, for use in a method of treating or lessening the severity in a subject of gut pain, wherein gut pain comprises inflammatory bowel disease pain, Crohn's disease pain, irritable bowel syndrome, endometriosis, polycyctic ovarian disease, salpingitis, cervicitis or interstitial cystitis pain.

In another aspect, the invention features a compound of the invention, or a pharmaceutically acceptable salt or pharmaceutical composition thereof, for use in a method of treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In another aspect, the invention features a compound of the invention, or a pharmaceutically acceptable salt or pharmaceutical composition thereof, for use in a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia.

In another aspect, the invention features a compound of the invention, or a pharmaceutically acceptable salt or pharmaceutical composition thereof, for use in a method of treating or lessening the severity in a subject of musculoskeletal pain. In some aspects, the musculoskeletal pain comprises osteoarthritis pain.

In another aspect, the invention features a compound of the invention, or a pharmaceutically acceptable salt or pharmaceutical composition thereof, for use in a method of treating or lessening the severity in a subject of musculoskeletal pain, wherein musculoskeletal pain comprises osteoarthritis pain, back pain, cold pain, burn pain or dental pain.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain, ankylosing spondylitis or vulvodynia.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises fibromyalgia pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises reflex sympathetic dystrophy pain.

In another aspect, the invention features a compound of the invention, or a pharmaceutically acceptable salt or pharmaceutical composition thereof, for use in a method of treating or lessening the severity in a subject of pathological cough.

In another aspect, the invention features a compound of the invention, or a pharmaceutically acceptable salt or pharmaceutical composition thereof, for use in a method of treating or lessening the severity in a subject of acute pain. In some aspects, the acute pain comprises acute post-operative pain.

In another aspect, the invention features a compound of the invention, or a pharmaceutically acceptable salt or pharmaceutical composition thereof, for use in a method of treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain).

In another aspect, the invention features a compound of the invention, or a pharmaceutically acceptable salt or pharmaceutical composition thereof, for use in a method of treating or lessening the severity in a subject of bunionectomy pain.

In another aspect, the invention features a compound of the invention, or a pharmaceutically acceptable salt or pharmaceutical composition thereof, for use in a method of treating or lessening the severity in a subject of shoulder arthroplasty pain or shoulder arthroscopy pain.

In another aspect, the invention features a compound of the invention, or a pharmaceutically acceptable salt or pharmaceutical composition thereof, for use in a method of treating or lessening the severity in a subject of herniorrhaphy pain.

In another aspect, the invention features a compound of the invention, or a pharmaceutically acceptable salt or pharmaceutical composition thereof, for use in a method of treating or lessening the severity in a subject of abdominoplasty pain.

In another aspect, the invention features a compound of the invention, or a pharmaceutically acceptable salt or pharmaceutical composition thereof, for use in a method of treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for use in a method of treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In another aspect, the invention features a compound of the invention, or a pharmaceutically acceptable salt or pharmaceutical composition thereof, for use in a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a compound of the invention, or a pharmaceutically acceptable salt or pharmaceutical composition thereof, for use in a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia rheumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodiscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarthropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention features a compound of the invention, or a pharmaceutically acceptable salt or pharmaceutical composition thereof, for use in a method of treating or lessening the severity in a subject of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain.

In another aspect, the invention features a compound of the invention, or a pharmaceutically acceptable salt or pharmaceutical composition thereof, for use in a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Manufacture of Medicaments

In another aspect, the invention provides the use of a compound of the invention, or a pharmaceutically acceptable salt or pharmaceutical composition thereof, for the manufacture of a medicament.

In another aspect, the invention provides the use of a compound of the invention, a pharmaceutically acceptable salt thereof, or a pharmaceutical composition thereof for the manufacture of a medicament for use in inhibiting a voltage-gated sodium channel. In another aspect, the voltage-gated sodium channel is Na_{V}1.8.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In yet another aspect, the invention provides the use of the compound, pharmaceutically acceptable salt, or pharmaceutical composition described herein for the manufacture of a medicament for use in treating or lessening the severity in a subject of gut pain, wherein gut pain comprises inflammatory bowel disease pain, Crohn's disease pain, irritable bowel syndrome, endometriosis, polycyctic ovarian disease, salpingitis, cervicitis or interstitial cystitis pain.

In yet another aspect, the invention provides a compound of the invention, a pharmaceutically acceptable salt thereof, or a pharmaceutical composition thereof for the manufacture of a medicament for use in treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy).

In yet another aspect, the invention provides the use of a compound of the invention, a pharmaceutically acceptable salt thereof, or a pharmaceutical composition thereof for the manufacture of a medicament for use in a treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic neuropathy.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain. In some aspects, the musculoskeletal pain comprises osteoarthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain, wherein musculoskeletal pain comprises osteoarthritis pain, back pain, cold pain, burn pain or dental pain.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain, ankylosing spondylitis or vulvodynia.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises fibromyalgia pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises reflex sympathetic dystrophy pain.

In yet another aspect, the invention provides the use of a compound of the invention, a pharmaceutically acceptable salt thereof, or a pharmaceutical composition thereof for the manufacture of a medicament for use in treating or lessening the severity in a subject of pathological cough.

In yet another aspect, the invention provides the use of a compound of the invention, a pharmaceutically acceptable salt thereof, or a pharmaceutical composition thereof for the manufacture of a medicament for use in treating or lessening the severity in a subject of acute pain. In some aspects, the acute pain comprises acute post-operative pain.

In yet another aspect, the invention provides the use of a compound of the invention, a pharmaceutically acceptable salt thereof, or a pharmaceutical composition thereof for the manufacture of a medicament for use in treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain).

In yet another aspect, the invention provides the use of a compound of the invention, a pharmaceutically acceptable salt thereof, or a pharmaceutical composition thereof for the manufacture of a medicament for use in treating or lessening the severity in a subject of herniorrhaphy pain.

In yet another aspect, the invention provides the use of a compound of the invention, a pharmaceutically acceptable salt thereof, or a pharmaceutical composition thereof for the manufacture of a medicament for use in treating or lessening the severity in a subject of bunionectomy pain.

In yet another aspect, the invention provides the use of a compound of the invention, a pharmaceutically acceptable salt thereof, or a pharmaceutical composition thereof for the manufacture of a medicament for use in treating or lessening the severity in a subject of shoulder arthroplasty pain or shoulder arthroscopy pain.

In yet another aspect, the invention provides the use of a compound of the invention, a pharmaceutically acceptable salt thereof, or a pharmaceutical composition thereof for the manufacture of a medicament for use in treating or lessening the severity in a subject of abdominoplasty pain.

In yet another aspect, the invention provides the use of a compound of the invention, a pharmaceutically acceptable salt thereof, or a pharmaceutical composition thereof for the manufacture of a medicament for use in treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for the manufacture of a medicament for use in treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In yet another aspect, the invention provides the use of a compound of the invention, a pharmaceutically acceptable salt thereof, or a pharmaceutical composition thereof for the manufacture of a medicament for use in combination with one or more additional therapeutic agents administered concurrently with, prior to, or subsequent to treatment with the compound or pharmaceutical composition. In some embodiments, the additional therapeutic agent is a sodium channel inhibitor.

In another aspect, the invention provides the use of a compound of the invention, a pharmaceutically acceptable salt thereof, or a pharmaceutical composition thereof for the manufacture of a medicament for use in treating or lessening the severity of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia rheumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodiscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarthropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention provides the use of a compound of the invention, a pharmaceutically acceptable salt thereof, or a pharmaceutical composition thereof for the manufacture of a medicament for use in treating or lessening the severity of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain.

In another aspect, the invention provides the use of a compound of the invention, a pharmaceutically acceptable salt thereof, or a pharmaceutical composition thereof for the manufacture of a medicament for use in treating or lessening the severity of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Administration of Compounds, Pharmaceutically Acceptable Salts, and Compositions

In certain embodiments of the invention, an “effective amount” of a compound of the invention, a pharmaceutically acceptable salt thereof, or a pharmaceutical composition thereof is that amount effective for treating or lessening the severity of one or more of the conditions recited above.

The compounds, salts, and compositions, according to the method of the invention, may be administered using any amount and any route of administration effective for treating or lessening the severity of one or more of the pain or non-pain diseases recited herein. The exact amount required will vary from subject to subject, depending on the species, age, and general condition of the subject, the severity of the condition, the particular agent, its mode of administration, and the like. The compounds, salts, and compositions of the invention are preferably formulated in dosage unit form for ease of administration and uniformity of dosage. The expression “dosage unit form” as used herein refers to a physically discrete unit of agent appropriate for the subject to be treated. It will be understood, however, that the total daily usage of the compounds, salts, and compositions of the invention will be decided by the attending physician within the scope of sound medical judgment. The specific effective dose level for any particular subject or organism will depend upon a variety of factors including the disorder being treated and the severity of the disorder; the activity of the specific compound or salt employed; the specific composition employed; the age, body weight, general health, sex and diet of the subject; the time of administration, route of administration, and rate of excretion of the specific compound or salt employed; the duration of the treatment; drugs used in combination or coincidental with the specific compound or salt employed, and like factors well known in the medical arts. The term “subject” or “patient,” as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the condition being treated. In certain embodiments, the compound, salts, and compositions of the invention may be administered orally or parenterally at dosage levels of about 0.001 mg/kg to about 1000 mg/kg, one or more times a day, effective to obtain the desired therapeutic effect.

Liquid dosage forms for oral administration include, but are not limited to, pharmaceutically acceptable emulsions, microemulsions, solutions, suspensions, syrups and elixirs. In addition to the active compound or salt, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of the compounds of the invention, it is often desirable to slow the absorption of the compounds from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compound or salt of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound or salt is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compound or salt can also be in microencapsulated form with one or more excipients as noted above. The solid dosage forms of tablets, dragees, capsules, pills, and granules can be prepared with coatings and shells such as enteric coatings, release-controlling coatings and other coatings well known in the pharmaceutical formulating art. In such solid dosage forms, the active compound or salt may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound or salt of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds of the invention are useful as inhibitors of voltage-gated sodium channels. In one embodiment, the compounds are inhibitors of Na_{V}1.8 and thus, without wishing to be bound by any particular theory, the compounds, salts, and compositions are particularly useful for treating or lessening the severity of a disease, condition, or disorder where activation or hyperactivity of Na_{V}1.8 is implicated in the disease, condition, or disorder. When activation or hyperactivity of Na_{V}1.8 is implicated in a particular disease, condition, or disorder, the disease, condition, or disorder may also be referred to as a “Na_{V}1.8-mediated disease, condition or disorder.” Accordingly, in another aspect, the invention provides a method for treating or lessening the severity of a disease, condition, or disorder where activation or hyperactivity of Na_{V}1.8 is implicated in the disease state.

The activity of a compound utilized in this invention as an inhibitor of Na_{V}1.8 may be assayed according to methods described generally in International Publication No. WO 2014/120808 A9 and U.S. Publication No. 2014/0213616 A1, both of which are incorporated by reference in their entirety, methods described herein, and other methods known and available to one of ordinary skill in the art.

### Additional Therapeutic Agents

It will also be appreciated that the compounds, salts, and pharmaceutically acceptable compositions of the invention can be employed in combination therapies, that is, the compounds, salts, and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.” For example, exemplary additional therapeutic agents include, but are not limited to: non-opioid analgesics (indoles such as Etodolac, Indomethacin, Sulindac, Tolmetin; naphthylalkanones such as Nabumetone; oxicams such as Piroxicam; para-aminophenol derivatives, such as Acetaminophen; propionic acids such as Fenoprofen, Flurbiprofen, Ibuprofen, Ketoprofen, Naproxen, Naproxen sodium, Oxaprozin; salicylates such as Aspirin, Choline magnesium trisalicylate, Diflunisal; fenamates such as meclofenamic acid, Mefenamic acid; and pyrazoles such as Phenylbutazone); or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infusion, neural blockade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Nineteenth Edition, Ed. Robert S. Porter and Justin L. Kaplan, Merck Sharp &Dohme Corp., a subsidiary of Merck & Co., Inc., 2011, and the Food and Drug Administration website, www.fda.gov, the entire contents of which are hereby incorporated by reference.

In another embodiment, additional appropriate therapeutic agents are selected from the following:
(1) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine, pentazocine, or difelikefalin;
(2) a nonsteroidal antiinflammatory drug (NSAID), e.g. aspirin, diclofenac, diflunisal, etodolac, fenbufen, fenoprofen, flufenisal, flurbiprofen, ibuprofen (including without limitation intravenous ibuprofen (e.g., Caldolor®)), indomethacin, ketoprofen, ketorolac (including without limitation ketorolac tromethamine (e.g., Toradol®)), meclofenamic acid, mefenamic acid, meloxicam, IV meloxicam (e.g., Anjeso®), nabumetone, naproxen, nimesulide, nitroflurbiprofen, olsalazine, oxaprozin, phenylbutazone, piroxicam, sulfasalazine, sulindac, tolmetin or zomepirac;
(3) a barbiturate sedative, e.g. amobarbital, aprobarbital, butabarbital, butalbital, mephobarbital, metharbital, methohexital, pentobarbital, phenobarbital, secobarbital, talbutal, thiamylal or thiopental;
(4) a benzodiazepine having a sedative action, e.g. chlordiazepoxide, clorazepate, diazepam, flurazepam, lorazepam, oxazepam, temazepam or triazolam;
(5) a histamine (H₁) antagonist having a sedative action, e.g. diphenhydramine, pyrilamine, promethazine, chlorpheniramine or chlorcyclizine;
(6) a sedative such as glutethimide, meprobamate, methaqualone or dichloralphenazone;
(7) a skeletal muscle relaxant, e.g. baclofen, carisoprodol, chlorzoxazone, cyclobenzaprine, methocarbamol or orphenadrine;
(8) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®), a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
(9) an alpha-adrenergic, e.g. doxazosin, tamsulosin, clonidine, guanfacine, dexmedetomidine, modafinil, or 4-amino-6,7-dimethoxy-2-(5-methane-sulfonamido-1,2,3,4-tetrahydroisoquinolin-2-yl)-5-(2-pyridyl) quinazoline;
(10) a tricyclic antidepressant, e.g. desipramine, imipramine, amitriptyline or nortriptyline;
(11) an anticonvulsant, e.g. carbamazepine (Tegretol®), lamotrigine, topiramate, lacosamide (Vimpat®) or valproate;
(12) atachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
(13) a muscarinic antagonist, e.g oxybutynin, tolterodine, propiverine, tropsium chloride, darifenacin, solifenacin, temiverine and ipratropium;
(14) a COX-2 selective inhibitor, e.g. celecoxib, rofecoxib, parecoxib, valdecoxib, deracoxib, etoricoxib, or lumiracoxib;
(15) a coal-tar analgesic, in particular paracetamol;
(16) a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
(17) a vanilloid receptor agonist (e.g. resinferatoxin or civamide) or antagonist (e.g. capsazepine, GRC-15300);
(18) a beta-adrenergic such as propranolol;
(19) a local anesthetic such as mexiletine;
(20) a corticosteroid such as dexamethasone;
(21) a 5-HT receptor agonist or antagonist, particularly a 5-HT_{1B/1D} agonist such as eletriptan, sumatriptan, naratriptan, zolmitriptan or rizatriptan;
(22) a 5-HT_{2A} receptor antagonist such as R(+)-alpha-(2,3-dimethoxy-phenyl)-1-[2-(4-fluorophenylethyl)]-4-piperidinemethanol (MDL-100907);
(23) a cholinergic (nicotinic) analgesic, such as isoprenaline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
(24) Tramadol®, Tramadol ER (Ultram ERR), IV Tramadol, Tapentadol ER (Nucynta®);
(25) a PDE5 inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
(26) an alpha-2-delta ligand such as gabapentin (Neurontin®), gabapentin GR (Gralise®), gabapentin, enacarbil (Horizant®), pregabalin (Lyrica®), 3-methyl gabapentin, (1[alpha],3[alpha],5[alpha])(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
(27) a cannabinoid such as KHK-6188;
(28) metabotropic glutamate subtype 1 receptor (mGluR1) antagonist;
(29) a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
(30) a noradrenaline (norepinephrine) reuptake inhibitor, such as maprotiline, lofepramine, mirtazepine, oxaprotiline, fezolamine, tomoxetine, mianserin, bupropion, bupropion metabolite hydroxybupropion, nomifensine and viloxazine (Vivalan®), especially a selective noradrenaline reuptake inhibitor such as reboxetine, in particular (S,S)-reboxetine;
(31) a dual serotonin-noradrenaline reuptake inhibitor, such as venlafaxine, venlafaxine metabolite O-desmethylvenlafaxine, clomipramine, clomipramine metabolite desmethylclomipramine, duloxetine (Cymbalta®), milnacipran and imipramine;
(32) an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-S-chloro-S-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, NXN-462, or guanidinoethyldisulfide;
(33) an acetylcholinesterase inhibitor such as donepezil;
(34) a prostaglandin E2 subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(15)-1-({[5-chloro-2-(3-fluorophenoxy)pyridin-3-yl]carbonyl}amino)ethyl]benzoic acid;
(35) a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870;
(36) a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl)-1,4-benzoquinone (CV-6504);
(37) a sodium channel blocker, such as lidocaine, lidocaine plus tetracaine cream (ZRS-201) or eslicarbazepine acetate;
(38) a Na_{V}1.7 blocker, such as XEN-402, XEN403, TV-45070, PF-05089771, CNV1014802, GDC-0276, RG7893 BIIB-074 (Vixotrigine), BIIB-095, ASP-1807, DSP-3905, OLP-1002, RQ-00432979, FX-301, DWP-1706, DWP-17061, IMB-110, IMB-111, IMB-112 and such as those disclosed in WO2011/140425 (US2011/306607); WO2012/106499 (US2012196869); WO2012/112743 (US2012245136); WO2012/125613 (US2012264749), WO2012/116440 (US2014187533), WO2011026240 (US2012220605), U.S. Pat. Nos. 8,883,840, 8,466,188, WO2013/109521 (US2015005304), CN111217776, WO2020/117626, WO2021/252822, WO2021/252818, WO2021/252820, WO2014/201173, WO2012/125973, WO2013/086229, WO2013/134518, WO2014/201206, or WO2016/141035 the entire contents of each application hereby incorporated by reference;
(38a) a Na_{V}1.7 blocker such as (2-benzylspiro[3,4-dihydropyrrolo[1,2-a]pyrazine-1,4′-piperidine]-1′-yl)-(4-isopropoxy-3-methyl-phenyl)methanone, 2,2,2-trifluoro-1-[1′-[3-methoxy-4-[2-(trifluoromethoxy)ethoxy]benzoyl]-2,4-dimethyl-spiro[3,4-dihydropyrrolo[1,2-a]pyrazine-1,4′-piperidine]-6-yl]ethanone, [8-fluoro-2-methyl-6-(trifluoromethyl)spiro[3,4-dihydropyrrolo[1,2-a]pyrazine-1,4′-piperidine]-1′-yl]-(4-isobutoxy-3-methoxy-phenyl)methanone, 1-(4-benzhydrylpiperazin-1-yl)-3-[2-(3,4-dimethylphenoxy)ethoxy]propan-2-ol, (4-butoxy-3-methoxy-phenyl)-[2-methyl-6-(trifluoromethyl)spiro[3,4-dihydropyrrolo[1,2-a]pyrazine-1,4′-piperidine]-1′-yl]methanone, [8-fluoro-2-methyl-6-(trifluoromethyl)spiro[3,4-dihydropyrrolo[1,2-a]pyrazine-1,4′-piperidine]-1′-yl]-(5-isopropoxy-6-methyl-2-pyridyl)methanone, (4-isopropoxy-3-methyl-phenyl)-[2-methyl-6-(1,1,2,2,2-pentafluoroethyl)spiro[3,4-dihydropyrrolo[1,2-a]pyrazine-1,4′-piperidine]-1′-yl]methanone, 5-[2-methyl-4-[2-methyl-6-(2,2,2-trifluoroacetyl)spiro[3,4-dihydropyrrolo[1,2-a]pyrazine-1,4′-piperidine]-1′-carbonyl]phenyl]pyridine-2-carbonitrile, (4-isopropoxy-3-methyl-phenyl)-[6-(trifluoromethyl)spiro[3,4-dihydro-2H-pyrrolo[1,2-a]pyrazine-1,4′-piperidine]-1′-yl]methanone, 2,2,2-trifluoro-1-[1′-[3-methoxy-4-[2-(trifluoromethoxy)ethoxy]benzoyl]-2-methyl-spiro[3,4-dihydropyrrolo[1,2-a]pyrazine-1,4′-piperidine]-6-yl]ethanone, 2,2,2-trifluoro-1-[1′-(5-isopropoxy-6-methyl-pyridine-2-carbonyl)-3,3-dimethyl-spiro[2,4-dihydropyrrolo[1,2-a]pyrazine-1,4′-piperidine]-6-yl]ethanone, 2,2,2-trifluoro-1-[1′-(5-isopentyloxypyridine-2-carbonyl)-2-methyl-spiro[3,4-dihydropyrrolo[1,2-a]pyrazine-1,4′-piperidine]-6-yl]ethanone, (4-isopropoxy-3-methoxy-phenyl)-[2-methyl-6-(trifluoromethyl)spiro[3,4-dihydropyrrolo[1,2-a]pyrazine-1,4′-piperidine]-1′-yl]methanone, 2,2,2-trifluoro-1-[1′-(5-isopentyloxypyridine-2-carbonyl)-2,4-dimethyl-spiro[3,4-dihydropyrrolo[1,2-a]pyrazine-1,4′-piperidine]-6-yl]ethanone, 1-[(3S)-2,3-dimethyl-1′-[4-(3,3,3-trifluoropropoxymethyl)benzoyl]spiro[3,4-dihydropyrrolo[1,2-a]pyrazine-1,4′-piperidine]-6-yl]-2,2,2-trifluoro-ethanone, [8-fluoro-2-methyl-6-(trifluoromethyl)spiro[3,4-dihydropyrrolo[1,2-a]pyrazine-1,4′-piperidine]-1′-yl]-[3-methoxy-4-[(1R)-1-methylpropoxy]phenyl]methanone, 2,2,2-trifluoro-1-[1′-(5-isopropoxy-6-methyl-pyridine-2-carbonyl)-2,4-dimethyl-spiro[3,4-dihydropyrrolo[1,2-a]pyrazine-1,4′-piperidine]-6-yl]ethanone, 1-[1′-[4-methoxy-3-(trifluoromethyl)benzoyl]-2-methyl-spiro[3,4-dihydropyrrolo[1,2-a]pyrazine-1,4′-piperidine]-6-yl]-2,2-dimethyl-propan-1-one, (4-isopropoxy-3-methyl-phenyl)-[2-methyl-6-(trifluoromethyl)spiro[3,4-dihydropyrrolo[1,2-a]pyrazine-1,4′-piperidine]-1′-yl]methanone, [2-methyl-6-(1-methylcyclopropanecarbonyl)spiro[3,4-dihydropyrrolo[1,2-a]pyrazine-1,4′-piperidine]-1′-yl]-[4-(3,3,3-trifluoropropoxymethyl)phenyl]methanone, 4-bromo-N-(4-bromophenyl)-3-[(1-methyl-2-oxo-4-piperidyl)sulfamoyl]benzamide or (3-chloro-4-isopropoxy-phenyl)-[2-methyl-6-(1,1,2,2,2-pentafluoroethyl)spiro[3,4-dihydropyrrolo[1,2-a]pyrazine-1,4′-piperidine]-1′-yl]methanone.
(39) a Na_{V}1.8 blocker, such as PF-04531083, PF-06372865 and such as those disclosed in WO2008/135826 (US2009048306), WO2006/011050 (US2008312235), WO2013/061205 (US2014296313), US20130303535, WO2013131018, U.S. Pat. No. 8,466,188, WO2013114250 (US2013274243), WO2014/120808 (US2014213616), WO2014/120815 (US2014228371) WO2014/120820 (US2014221435), WO2015/010065 (US20160152561), WO2015/089361 (US20150166589), WO2019/014352 (US20190016671), WO2018/213426, WO2020/146682, WO2020/146612, WO2020/014243, WO2020/014246, WO2020/092187, WO2020/092667 (US2020140411), WO2020/144375, WO2020/261114, WO2020/140959, WO2020/151728, WO2021/032074, WO2021/047622 (CN112479996), WO2021/257490, WO/2021/257420, WO2021/257418, WO2022/263498, WO2022/235558, WO2022/235859, CN112390745, CN111808019, CN112225695, CN112457294, CN112300051, CN112300069, CN112441969, and CN114591293, the entire contents of each application hereby incorporated by reference;
(39a) a Na_{V}.8 blocker such as 4,5-dichloro-2-(4-fluoro-2-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-4-(perfluoroethyl)benzamide, 4,5-dichloro-2-(4-fluorophenoxy)-N-(2-oxo-1,2-dihydropyridin-4-yl)benzamide, 4,5-dichloro-2-(3-fluoro-4-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-5-(trifluoromethyl)benzamide, N-(2-oxo-1,2-dihydropyridin-4-yl)-2-(4-(trifluoromethoxy)phenoxy)-4-(trifluoromethyl)benzamide, 2-(4-fluorophenoxy)-N-(2-oxo-1,2-dihydropyridin-4-yl)-4-(perfluoroethyl)benzamide, 5-chloro-2-(4-fluoro-2-methoxyphenoxy)-N-(2-oxo-1,2-dihydropyridin-4-yl)benzamide, N-(2-oxo-1,2-dihydropyridin-4-yl)-2-(4-(trifluoromethoxy)phenoxy)-5-(trifluoromethyl)benzamide, 2-(4-fluoro-2-methylphenoxy)-N-(2-oxo-1,2-dihydropyridin-4-yl)-5-(trifluoromethyl)benzamide, 2-(2-chloro-4-fluorophenoxy)-N-(2-oxo-1,2-dihydropyridin-4-yl)-5-(trifluoromethyl)benzamide, 5-chloro-2-(4-fluoro-2-methylphenoxy)-N-(2-oxo-1,2-dihydropyridin-4-yl)benzamide, 4-chloro-2-(4-fluoro-2-methylphenoxy)-N-(2-oxo-1,2-dihydropyridin-4-yl)benzamide, 5-chloro-2-(2-chloro-4-fluorophenoxy)-N-(2-oxo-1,2-dihydropyridin-4-yl)benzamide, 2-((5-fluoro-2-hydroxybenzyl)oxy)-N-(2-oxo-1,2-dihydropyridin-4-yl)-4-(trifluoromethyl)benzamide, N-(2-oxo-1,2-dihydropyridin-4-yl)-2-(o-tolyloxy)-5-(trifluoromethyl)benzamide, 2-(2,4-difluorophenoxy)-N-(2-oxo-1,2-dihydropyridin-4-yl)-4-(trifluoromethyl)benzamide, N-(2-oxo-1,2-dihydropyridin-4-yl)-2-(2-(trifluoromethoxy)phenoxy)-5-(trifluoromethyl)benzamide, 2-(4-fluorophenoxy)-N-(2-oxo-1,2-dihydropyridin-4-yl)-5-(trifluoromethyl)benzamide, 2-(4-fluoro-2-methyl-phenoxy)-N-(2-oxo-1H-pyridin-4-yl)-4-(trifluoromethyl)benzamide, [4-[[2-(4-fluoro-2-methyl-phenoxy)-4-(trifluoromethyl)benzoyl]amino]-2-oxo-1-pyridyl]methyl dihydrogen phosphate, 2-(4-fluoro-2-(methyl-d₃)phenoxy)-N-(2-oxo-1,2-dihydropyridin-4-yl)-4-(trifluoromethyl)benzamide, (4-(2-(4-fluoro-2-(methyl-d₃)phenoxy)-4-(trifluoromethyl)benzamido)-2-oxopyridin-1(2H)-yl)methyl dihydrogen phosphate, 3-(4-fluoro-2-methoxyphenoxy)-N-(3-(methylsulfonyl)phenyl)quinoxaline-2-carboxamide, 3-(2-chloro-4-fluorophenoxy)-N-(3-sulfamoylphenyl)quinoxaline-2-carboxamide, 3-(2-chloro-4-methoxyphenoxy)-N-(3-sulfamoylphenyl)quinoxaline-2-carboxamide, 3-(4-chloro-2-methoxyphenoxy)-N-(3-sulfamoylphenyl)quinoxaline-2-carboxamide, 4-(3-(4-(trifluoromethoxy)phenoxy)quinoxaline-2-carboxamido)picolinic acid, 2-(2,4-difluorophenoxy)-N-(3-sulfamoylphenyl)quinoline-3-carboxamide, 2-(4-fluoro-2-methoxyphenoxy)-N-(3-sulfamoylphenyl)quinoline-3-carboxamide, 3-(2,4-difluorophenoxy)-N-(3-sulfamoylphenyl)quinoxaline-2-carboxamide, N-(3-sulfamoylphenyl)-2-(4-(trifluoromethoxy)phenoxy)quinoline-3-carboxamide, N-(3-sulfamoylphenyl)-3-(4-(trifluoromethoxy)phenoxy)quinoxaline-2-carboxamide, 3-(4-chloro-2-methylphenoxy)-N-(3-sulfamoylphenyl)quinoxaline-2-carboxamide, 5-(3-(4-(trifluoromethoxy)phenoxy)quinoxaline-2-carboxamido)picolinic acid, 3-(4-fluoro-2-methoxyphenoxy)-N-(2-oxo-2,3-dihydro-1H-benzo[d]imidazol-5-yl)quinoxaline-2-carboxamide, 3-(4-fluoro-2-methoxyphenoxy)-N-(pyridin-4-yl)quinoxaline-2-carboxamide, 3-(4-fluorophenoxy)-N-(3-sulfamoylphenyl)quinoxaline-2-carboxamide, N-(3-cyanophenyl)-3-(4-fluoro-2-methoxyphenoxy)quinoxaline-2-carboxamide, N-(4-carbamoylphenyl)-3-(4-fluoro-2-methoxyphenoxy)quinoxaline-2-carboxamide, 4-(3-(4-(trifluoromethoxy)phenoxy)quinoxaline-2-carboxamido)benzoic acid, N-(4-cyanophenyl)-3-(4-fluoro-2-methoxyphenoxy)quinoxaline-2-carboxamide, 5-(4,5-dichloro-2-(4-fluoro-2-methoxyphenoxy)benzamido)picolinic acid, 5-(2-(2,4-dimethoxyphenoxy)-4,6-bis(trifluoromethyl)benzamido)picolinic acid, 4-(4,5-dichloro-2-(4-fluoro-2-methoxyphenoxy)benzamido)benzoic acid, 5-(2-(4-fluoro-2-methoxyphenoxy)-4,6-bis(trifluoromethyl)benzamido)picolinic acid, 4-(2-(4-fluoro-2-methoxyphenoxy)-4-(perfluoroethyl)benzamido)benzoic acid, 5-(2-(4-fluoro-2-methoxyphenoxy)-4-(perfluoroethyl)benzamido)picolinic acid, 4-(2-(4-fluoro-2-methylphenoxy)-4-(trifluoromethyl)benzamido)benzoic acid, 5-(4,5-dichloro-2-(4-fluoro-2-methoxyphenoxy)benzamido)picolinic acid, 4-(2-(2-chloro-4-fluorophenoxy)-4-(perfluoroethyl)benzamido)benzoic acid, 4-(2-(4-fluoro-2-methylphenoxy)-4-(perfluoroethyl)benzamido)benzoic acid, 4-(4,5-dichloro-2-(4-(trifluoromethoxy)phenoxy)benzamido)benzoic acid, 4-(4,5-dichloro-2-(4-chloro-2-methylphenoxy)benzamido)benzoic acid, 5-(4-(tert-butyl)-2-(4-fluoro-2-methoxyphenoxy)benzamido)picolinic acid, 5-(4,5-dichloro-2-(4-(trifluoromethoxy)phenoxy)benzamido)picolinic acid, 4-(4,5-dichloro-2-(4-fluoro-2-methylphenoxy)benzamido)benzoic acid, 5-(4,5-dichloro-2-(2,4-dimethoxyphenoxy)benzamido)picolinic acid, 5-(4,5-dichloro-2-(2-chloro-4-fluorophenoxy)benzamido)picolinic acid, 5-(4,5-dichloro-2-(4-fluoro-2-methylphenoxy)benzamido)picolinic acid, 4-(4,5-dichloro-2-(4-chloro-2-methoxyphenoxy)benzamido)benzoic acid, 5-(4,5-dichloro-2-(2,4-difluorophenoxy)benzamido)picolinic acid, 2-(4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-6-(trifluoromethyl)benzamide, 2-(2-chloro-4-fluorophenoxy)-5-(difluoromethyl)-N-(3-sulfamoylphenyl)benzamide, 2-(4-fluorophenoxy)-4-(perfluoroethyl)-N-(3-sulfamoylphenyl)benzamide, 2-(4-chloro-2-methoxyphenoxy)-4-(perfluoroethyl)-N-(3-sulfamoylphenyl)benzamide, 2-(4-fluoro-2-methoxyphenoxy)-N-(3-sulfamoylphenyl)-5-(trifluoromethyl)benzamide, 5-chloro-2-(4-fluoro-2-methylphenoxy)-N-(3-sulfamoylphenyl)benzamide, 4,5-dichloro-2-(4-fluoro-2-methoxyphenoxy)-N-(3-sulfamoylphenyl)benzamide, 2,4-dichloro-6-(4-chloro-2-methoxyphenoxy)-N-(3-sulfamoylphenyl)benzamide, 2,4-dichloro-6-(4-fluoro-2-methylphenoxy)-N-(3-sulfamoylphenyl)benzamide, 2-(4-fluoro-2-methoxyphenoxy)-N-(3-sulfamoylphenyl)-4,6-bis(trifluoromethyl)benzamide, 2-(4-fluoro-2-methylphenoxy)-N-(3-sulfamoylphenyl)-4,6-bis(trifluoromethyl)benzamide, 5-chloro-2-(2-chloro-4-fluorophenoxy)-N-(3-sulfamoylphenyl)benzamide, 2-(4-fluoro-2-methoxyphenoxy)-N-(3-sulfamoylphenyl)-4-(trifluoromethoxy)benzamide, 2-(4-fluoro-2-methoxyphenoxy)-N-(3-sulfamoylphenyl)-4-(trifluoromethyl)benzamide, 4,5-dichloro-2-(4-fluorophenoxy)-N-(3-sulfamoylphenyl)benzamide, 2-(4-fluoro-2-methoxyphenoxy)-4-(perfluoroethyl)-N-(3-sulfamoylphenyl)benzamide, 5-fluoro-2-(4-fluoro-2-methylphenoxy)-N-(3-sulfamoylphenyl)benzamide, 2-(2-chloro-4-fluorophenoxy)-4-cyano-N-(3-sulfamoylphenyl)benzamide, N-(3-sulfamoylphenyl)-2-(4-(trifluoromethoxy)phenoxy)-4-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethyl)benzamide, N-(3-carbamoyl-4-fluoro-phenyl)-2-fluoro-6-[2-methoxy-4-(trifluoromethoxy)phenoxy]-3-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethoxy)benzamide, 4-[[2-fluoro-6-[2-methoxy-4-(trifluoromethoxy)phenoxy]-3-(trifluoromethyl)benzoyl]amino]pyridine-2-carboxamide, 4-[[3-chloro-2-fluoro-6-[2-methoxy-4-(trifluoromethoxy)phenoxy]benzoyl]amino]pyridine-2-carboxamide, 4-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-3-(difluoromethyl)-2-fluoro-6-[2-methoxy-4-(trifluoromethoxy)phenoxy]benzamide, 4-[[2-fluoro-6-[2-(trideuteriomethoxy)-4-(trifluoromethoxy)phenoxy]-3-(trifluoromethoxy)benzoyl]amino]pyridine-2-carboxamide, N-(3-carbamoyl-4-fluoro-phenyl)-6-[2-chloro-4-(trifluoromethoxy)phenoxy]-2-fluoro-3-(trifluoromethyl)benzamide, N-(3-carbamoyl-4-fluoro-phenyl)-2-fluoro-6-[2-methyl-4-(trifluoromethoxy)phenoxy]-3-(trifluoromethyl)benzamide, N-(3-carbamoyl-4-fluoro-phenyl)-2,3,4-trifluoro-6-[2-methoxy-4-(trifluoromethoxy)phenoxy]benzamide, N-(2-carbamoyl-4-pyridyl)-3-fluoro-5-[2-methoxy-4-(trifluoromethoxy)phenoxy]-2-(trifluoromethyl)pyridine-4-carboxamide, 4-[[6-[2-(difluoromethoxy)-4-(trifluoromethoxy)phenoxy]-2-fluoro-3-(trifluoromethyl)benzoyl]amino]pyridine-2-carboxamide, N-(3-carbamoyl-4-fluoro-phenyl)-6-[3-chloro-4-(trifluoromethoxy)phenoxy]-2-fluoro-3-(trifluoromethyl)benzamide, N-(3-carbamoyl-4-fluoro-phenyl)-2-fluoro-6-[4-(trifluoromethoxy)phenoxy]-3-(trifluoromethyl)benzamide, N-(4-carbamoyl-3-fluoro-phenyl)-2-fluoro-6-[2-methoxy-4-(trifluoromethoxy)phenoxy]-3-(trifluoromethyl)benzamide, 4-[[2-fluoro-6-[2-(trideuteriomethoxy)-4-(trifluoromethoxy)phenoxy]-4-(trifluoromethyl)benzoyl]amino]pyridine-2-carboxamide, N-(3-carbamoyl-4-fluoro-phenyl)-2-fluoro-6-[3-fluoro-4-(trifluoromethoxy)phenoxy]-3-(trifluoromethyl)benzamide, N-(3-carbamoyl-4-fluoro-phenyl)-2-[2-methoxy-4-(trifluoromethoxy)phenoxy]-5-(1,1,2,2,2-pentafluoroethyl)benzamide, 4-[[4-(difluoromethoxy)-2-fluoro-6-[2-methoxy-4-(trifluoromethoxy)phenoxy]benzoyl]amino]pyridine-2-carboxamide, N-(3-carbamoyl-4-fluoro-phenyl)-2-fluoro-6-[2-fluoro-4-(trifluoromethoxy)phenoxy]-3-(trifluoromethyl)benzamide, 4-[[4-cyclopropyl-2-fluoro-6-[2-methoxy-4-(trifluoromethoxy)phenoxy]benzoyl]amino]pyridine-2-carboxamide, N-(3-carbamoyl-4-fluoro-phenyl)-5-fluoro-2-[2-methoxy-4-(trifluoromethoxy)phenoxy]-4-(trifluoromethyl)benzamide, 5-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-2-fluoro-6-(4-fluorophenoxy)-3-(trifluoromethyl)benzamide, or 4-[[2-fluoro-6-[3-fluoro-2-methoxy-4-(trifluoromethoxy)phenoxy]-3-(trifluoromethyl)benzoyl]amino]pyridine-2-carboxamide;
(40) a combined Na_{V}1.7 and Na_{V}1.8 blocker, such as DSP-2230, Lohocla201 or BL-1021;
(41) a 5-HT3 antagonist, such as ondansetron;
(42) a TPRV 1 receptor agonist, such as capsaicin (NeurogesX®, Qutenza®); and the pharmaceutically acceptable salts and solvates thereof,
(43) a nicotinic receptor antagonist, such as varenicline;
(44) an N-type calcium channel antagonist, such as Z-160;
(45) a nerve growth factor antagonist, such as tanezumab;
(46) an endopeptidase stimulant, such as senrebotase;
(47) an angiotensin II antagonist, such as EMA-401;
(48) acetaminophen (including without limitation intravenous acetaminophen (e.g., Ofirmev®));
(49) bupivacaine (including without limitation bupivacaine liposome injectable suspension (e.g., Exparel®) bupivacaine ER (Posimir), bupivacaine collagen (Xaracoll) and transdermal bupivacaine (Eladur®)); and
(50) bupivacaine and meloxicam combination (e.g., HTX-011).

In one embodiment, the additional appropriate therapeutic agents are selected from V-116517, Pregabalin, controlled release Pregabalin, Ezogabine (Potiga®). Ketamine/amitriptyline topical cream (Amiket®), AVP-923, Perampanel (E-2007), Ralfinamide, transdermal bupivacaine (Eladur®), CNV1014802, JNJ-10234094 (Carisbamate), BMS-954561 or ARC-4558.

In another embodiment, the additional appropriate therapeutic agents are selected from N-(6-amino-5-(2,3,5-trichlorophenyl)pyridin-2-yl)acetamide; N-(6-amino-5-(2-chloro-5-methoxyphenyl)pyridin-2-yl)-1-methyl-1H-pyrazole-5-carboxamide; or 3-((4-(4-(trifluoromethoxy)phenyl)-1H-imidazol-2-yl)methyl)oxetan-3-amine.

In another embodiment, the additional therapeutic agent is selected from a GlyT2/5HT2 inhibitor, such as Operanserin (VVZ149), a TRPV modulator such as CA008, CMX-020, NE06860, FTABS, CNTX4975, MCP101, MDR16523, or MDR652, a EGR1 inhibitor such as Brivoglide (AYX1), an NGF inhibitor such as Tanezumab, Fasinumab, ASP6294, MEDI7352, a Mu opioid agonist such as Cebranopadol, NKTR181 (oxycodegol), a CB-1 agonist such as NE01940 (AZN1940), an imidazoline 12 agonist such as CR4056 or a p75NTR-Fc modulator such as LEVI-04.

In another embodiment, the additional therapeutic agent is oliceridine or ropivacaine (TLC590).

In another embodiment, the additional therapeutic agent is a Na_{V}1.7 blocker such as ST-2427, ST-2578, and/or those disclosed in WO2010/129864, WO2015/157559, WO2017/059385, WO2018/183781, WO2018/183782, WO2020/072835, and/or WO 2022/036297 the entire contents of each application hereby incorporated by reference.

In another embodiment, the additional therapeutic agent is ASP18071, CC-8464, ANP-230, ANP-231, NOC-100, NTX-1175, ASN008, NW3509, AM-6120, AM-8145, AM-0422, BL-017881. NTM-006, Opiranserin (Unafra™), brivoligide, SR419, NRD.E1, LX9211, LY3016859, ISC-17536, NFX-88, LAT-8881, AP-235, NYX 2925, CNTX-6016, S-600918, S-637880, RQ-00434739, KLS-2031, MEDI 7352, or XT-150.

In another embodiment, the additional therapeutic agent is Olinvyk, Zynrelef, Seglentis, Neumentum, Nevakar, HTX-034, CPL-01, ACP-044, HRS-4800, Tarlige, BAY2395840, LY3526318, Eliapixant, TRV045, RTA901, NRD1355-E1, MT-8554, LY3556050, AP-325, tetrodotoxin, Otenaproxesul, CFTX-1554, Funapide, iN1011-N17, JMKX000623/ODM-111, ETX-801, OLP-1002, ANP-230/DSP-2230, iN1011-N17, DSP-3905 or ACD440.

In another embodiment, the additional therapeutic agent is a sodium channel inhibitor (also known as a sodium channel blocker), such as the Na_{V}1.7 and Na_{V}1.8 blockers identified above.

The amount of additional therapeutic agent present in the compositions of this invention may be no more than the amount that would normally be administered in a composition comprising that therapeutic agent as the only active agent. The amount of additional therapeutic agent in the presently disclosed compositions may range from about 10% to 100% of the amount normally present in a composition comprising that agent as the only therapeutically active agent.

The compounds and salts of this invention or pharmaceutically acceptable compositions thereof may also be incorporated into compositions for coating an implantable medical device, such as prostheses, artificial valves, vascular grafts, stents and catheters. Accordingly, the invention, in another aspect, includes a composition for coating an implantable device comprising a compound or salt of the invention as described generally above, and in classes and subclasses herein, and a carrier suitable for coating said implantable device. In still another aspect, the invention includes an implantable device coated with a composition comprising a compound or salt of the invention as described generally above, and in classes and subclasses herein, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

Another aspect of the invention relates to inhibiting Na_{V}1.8 activity in a biological sample or a subject, which method comprises administering to the subject, or contacting said biological sample with a compound of the invention, a pharmaceutically acceptable salt thereof, or a pharmaceutical composition thereof. The term “biological sample,” as used herein, includes, without limitation, cell cultures or extracts thereof; biopsied material obtained from a mammal or extracts thereof; and blood, saliva, urine, feces, semen, tears, or other body fluids or extracts thereof.

Inhibition of Na_{V}1.8 activity in a biological sample is useful for a variety of purposes that are known to one of skill in the art. Examples of such purposes include, but are not limited to, the study of sodium channels in biological and pathological phenomena; and the comparative evaluation of new sodium channel inhibitors.

### Synthesis of the Compounds of the Invention

The compounds of the invention can be prepared from known materials by the methods described in the Examples, other similar methods, and other methods known to one skilled in the art. As one skilled in the art would appreciate, the functional groups of the intermediate compounds in the methods described below may need to be protected by suitable protecting groups. Protecting groups may be added or removed in accordance with standard techniques, which are well-known to those skilled in the art. The use of protecting groups is described in detail in T. G. M. Wuts et al., Greene's Protective Groups in Organic Synthesis (4th ed. 2006).

### Radiolabeled Analogs of the Compounds of the Invention

In another aspect, the invention relates to radiolabeled analogs of the compounds of the invention. As used herein, the term “radiolabeled analogs of the compounds of the invention” refers to compounds that are identical to the compounds of the invention, as described herein, including all embodiments thereof, except that one or more atoms has been replaced with a radioisotope of the atom present in the compounds of the invention.

As used herein, the term “radioisotope” refers to an isotope of an element that is known to undergo spontaneous radioactive decay. Examples of radioisotopes include ³H, ¹⁴C, ³²P, ³⁵S, ¹⁸F, ³⁶Cl, and the like, as well as the isotopes for which a decay mode is identified in V. S. Shirley & C. M. Lederer, Isotopes Project, Nuclear Science Division, Lawrence Berkeley Laboratory, Table of Nuclides (January 1980).

The radiolabeled analogs can be used in a number of beneficial ways, including in various types of assays, such as substrate tissue distribution assays. For example, tritium (³H)- and/or carbon-14 (¹⁴C)-labeled compounds may be useful for various types of assays, such as substrate tissue distribution assays, due to relatively simple preparation and excellent detectability.

In another aspect, the invention relates to pharmaceutically acceptable salts of the radiolabeled analogs, in accordance with any of the embodiments described herein in connection with the compounds of the invention.

In another aspect, the invention relates to pharmaceutical compositions comprising the radiolabeled analogs, or pharmaceutically acceptable salts thereof, and a pharmaceutically acceptable carrier, adjuvant or vehicle, in accordance with any of the embodiments described herein in connection with the compounds of the invention.

In another aspect, the invention relates to methods of inhibiting voltage-gated sodium channels and methods of treating or lessening the severity of various diseases and disorders, including pain, in a subject comprising administering an effective amount of the radiolabeled analogs, pharmaceutically acceptable salts thereof, and pharmaceutical compositions thereof, in accordance with any of the embodiments described herein in connection with the compounds of the invention.

In another aspect, the invention relates to radiolabeled analogs, pharmaceutically acceptable salts thereof, and pharmaceutical compositions thereof, for use, in accordance with any of the embodiments described herein in connection with the compounds of the invention.

In another aspect, the invention relates to the use of the radiolabeled analogs, or pharmaceutically acceptable salts thereof, and pharmaceutical compositions thereof, for the manufacture of medicaments, in accordance with any of the embodiments described herein in connection with the compounds of the invention.

In another aspect, the radiolabeled analogs, pharmaceutically acceptable salts thereof, and pharmaceutical compositions thereof, can be employed in combination therapies, in accordance with any of the embodiments described herein in connection with the compounds of the invention.

### EXAMPLES

General methods. ¹H NMR spectra were obtained as solutions in an appropriate deuterated solvent such as dimethyl sulfoxide-d₆ (DMSO-d₆).

LCMS Methods. Compound purity, retention time, and electrospray mass spectrometry (ESI-MS) data were determined by LC/MS analysis. LC/MS determinations, unless otherwise indicated, were carried out using one of the following chromatographic conditions:
1) Waters BEH C₈ (1.7 μm, 2.1×50 mm) 2 to 98% acetonitrile in water (10 mM ammonium formate, pH 9), 45° C., flow rate 0.6 mL/min over 5.0 min;
2) Kinetex EVO C₁₈ (2.6 μm, 2.1×50 mm) 2 to 98% acetonitrile in water (10 mM ammonium formate, pH 9), 45° C., flow rate 0.7 mL/min over 4.0 min;
3) Kinetex EVO C₁₈ (2.6 μm 2.1×50 mm) 2 to 98% acetonitrile in water (10 mM ammonium formate, pH 9), 45° C., flow rate 1.0 mL/min over 1.5 min;
4) Waters Acquity UPLC BEH C₁₈ (1.7 μm, 30×2.1 mm) 1 to 99% acetonitrile (0.035% TFA) in water (0.05% TFA), 60° C., flow rate=1.5 mL/min over 3 min;
5) Kinetex Polar C₁₈ (2.6 μm, 3.0×50 mm) 5 to 95% acetonitrile in water (0.1% formic acid), flow rate 1.2 mL/min over 6 min;
6) SunFire C₁₈ (3.5 μm, 75×4.6 mm) initial 5 to 95% acetonitrile in water (0.1% formic acid) for 1 min then linear gradient to 95% acetonitrile for 5 min. 45° C., flow rate 1.5 mL/min over 6 min;
7) XBridge C₁₈ (5 μm, 4.6×75 mm) initial gradient 5 to 95% acetonitrile (NH₄HCO₃), 6 min run with 1 min equilibration gradient 0 to 3 min at 95% acetonitrile and hold for 3 min, flow rate 1.5 mL/min;
8) Waters CSH C₁₈ (1.7 m, 2.1×50 mm) 2 to 98% acetonitrile in water (0.1% TFA, pH 2), 45° C., flow rate 0.6 mL/min over 5.0 min;
9) Waters CSH C₁₈ (1.7 μm, 2.1×50 mm) 2 to 95% acetonitrile in water (0.1% formic acid), 40° C., flow rate 0.8 mL/min over 4.6 min;
10) Waters BEH C₁₈ (2.5 μm, 2.1×50 mm) 2 to 95% acetonitrile in water (0.1% NH₃), 40° C., flow rate 0.8 mL/min over 4.6 min;
11) Waters BEH C₁₈ (3.5 μm, 75×4.6 mm) initial gradient 5 to 95% acetonitrile in water (0.1% formic acid) then linear gradient to 95% acetonitrile for 4 min, hold for 2 min at 95% acetonitrile, 45° C., flow rate 1.5 mL/min over 6 min;
12) Waters BEH C₁₈ (2.5 μm, 2.1×50 mm) 2 to 50% acetonitrile in water (0.1% NH₃), 40° C., flow rate 0.8 mL/min over 4.6 min;
13) Waters CSH C₁₈ (1.7 μm, 2.1×50 mm) 2 to 98% acetonitrile in water (0.1% TFA), 45° C., flow rate 1.0 mL/min over 1.5 min;
14) Waters CSH C₁₈ (1.7 μm, 2.1×50 mm) 2 to 95% acetonitrile in water (0.1% formic acid), 40° C., flow rate 0.8 mL/min over 1.4 min;
15) YMC Triart C₁₈ (3 μm, 33×2.1 mm) 2 to 98% acetonitrile in water (5 mM NH₄OAc), flow rate 1.0 mL/min over 3 min;
16) Waters BEH C₁₈ (2.5 μm, 2.1×50 mm) 2 to 95% acetonitrile in water (0.1% NH₃), 40° C., flow rate 0.8 mL/min over 1.4 min;
17) Waters Acquity UPLC BEH C₁₈ (1.7 μm, 30×2.1 mm) 1 to 99% acetonitrile (0.035% TFA) in water (0.05% TFA), 60° C., flow rate=1.5 mL/min over 5 min;
18) Waters BEH C₁₈ (2.5 μm, 2.1×50 mm) 20 to 70% acetonitrile in water (0.1% NH₃), 40° C., flow rate 0.8 mL/min over 4.60 min;
19) Kinetex Polar C₁₈ (2.6 μm, 3.0×50 mm) 5 to 95% acetonitrile in water (0.1% formic acid), flow rate 1.2 mL/min over 3 min;
20) Waters Acquity UPLC BEH C₁₈ column (1.7 μm, 30×2.1 mm) 1 to 99% acetonitrile (0.035% TFA) in water (0.05% TFA), 60° C., flow rate=1.5 mL/min over 1 min;
21) YMC Triart C₁₈ (3 μm, 33×2.1 mm) 2 to 98% acetonitrile in water (0.05% formic acid), flow rate 1.0 mL/min over 3 min;
22) Waters Acquity UPLC BEH C₁₈ (1.7 μm, 30×2.1 mm) 1 to 99% acetonitrile (0.05% ammonium formate) in water (0.05% ammonium formate), 60° C., flow rate=1.5 mL/min over 5 min;
23) Waters CSH C₁₈ (1.7 μm, 2.1×50 mm) 2 to 98% acetonitrile in water (0.1% TFA), 45° C., flow rate 0.6 mL/min over 4.0 min;
24) Acquity BEH C₈ (1.7 μm, 50×2.1 mm) 2 to 98% 90:10 acetonitrile:water (0.05% formic acid), flow rate 0.8 mL/min over 3 min;
25) XBridge C₁₈ (5 μm, 50×4.6 mm) 10 to 90% acetonitrile in water (10 mM NH₄OAc), flow rate 1.2 mL/min over 6 min;
26) YMC Triart C₁₈ (3 μm, 33×2.1 mm) 5 to 95% acetonitrile in water (0.05% formic acid), flow rate 1.0 mL/min over 12 min;
27) Waters BEH C₈ (1.7 μm, 2.1×50 mm) 50 to 95% acetonitrile in water (0.1% NH₃), 40° C., flow rate 0.8 mL/min over 1.4 min.

### ABBREVIATIONS

Unless otherwise noted, or where the context dictates otherwise, the following abbreviations shall be understood to have the following meanings:

| Abbreviation | Meaning |
|---|---|
| NMR | Nuclear magnetic resonance |
| ESI-MS | Electrospray mass spectrometry |
| LC/MS | Liquid chromatography-mass spectrometry |
| UPLC | Ultra performance liquid chromatography |
| HPLC/MS/MS | High performance liquid chromatography/tandem mass spectrometry |
| IS | Internal standard |
| HPLC | High performance liquid chromatography |
| SFC | Supercritical fluid chromatography |
| ESI | Electrospray ionization |
| kg | Kilogram |
| g | Grams |
| mg | Milligrams |
| L | Liter(s) |
| mL | Milliliters |
| μL | Microliters |
| nL | Nanoliters |
| mol | Mole |
| mmol | Millimoles |
| hr, h | Hours |
| min | Minutes |
| ms | Millisecond |
| mm | Millimeters |
| μm | Micrometers |
| nm | Nanometer |
| MHz | Megahertz |
| Hz | Hertz |
| N | Normal (concentration) |
| M | Molar (concentration) |
| mM | Millimolar (concentration) |
| μM | Micromolar (concentration) |
| ppm | Parts per million |
| % w/v | Weight-volume concentration |
| % w/w | Weight-weight concentration |
| Ac₂O | Acetic anhydride |
| BnBr | Benzyl bromide |
| t-BuOH | Tert-butyl alcohol |
| CDI | 1,1′-Carbonyldiimidazole |
| DAST | (Diethylamino)sulfur trifluoride |
| DCM | Dichloromethane |
| DCE | Dichloroethane |
| DIAD | Diisopropyl azodicarboxylate |
| DIBAL | Diisobutylaluminium hydride |
| DIEA, DIPEA | N, N-Diisopropyl ethyl amine |
| DMA | N,N-Dimethylacetamide |
| DMAP | Dimethylaminopyridine |
| DMF | N,N-Dimethylformamide |
| DMSO | Dimethyl sulfoxide |
| DRG | Dorsal root ganglia |
| EtOH | Ethanol |
| EtOAc | Ethyl acetate |
| HATU | 1-[Bis(dimethylamino)methylene]-1H-1,2,3-triazolo[4,5-b]pyridinium |
| | 3-oxide hexafluorophosphate |
| EDCI | 1-Ethyl-3-(3-dimethylaminopropyl)carbodiimide |
| T3P | Propylphosphonic anhydride, i.e., 2,4,6-tripropyl-1,3,5,2,4,6- |
| | trioxatriphosphinane 2,4,6-trioxide |
| mCPBA | Meta-Chloroperoxybenzoic acid |
| MeOH | Methanol |
| MsCl | Methanesulfonyl chloride |
| MTBE | Methyl tert-butyl ether |
| NCS | N-Chlorosuccinimide |
| NIS | N-Todosuccinimide |
| NMP | N-Methylpyrrolidone |
| PdCl₂(dtbpf) | 1,1′-Bis(di-tert-butylphosphino)ferrocene palladium dichloride |
| PTSA | Para-tolucnesulfonic acid |
| STAB | Sodium triacetoxyborohydride |
| TBAF | Tetrabutylammonium fluoride |
| TBSOTf | Tert-Butyldimethylsilyl trifluoromethanesulfonate |
| TCFH | Chloro-N,N,N′,N′-tetramethylformamidinium hexafluorophosphate |
| THF | Tetrahydrofuran |
| TEA | Triethylamine |
| Tf₂O | Trifluoromethanesulfonic anhydride |
| TFA | Trifluoroacetic acid |
| TMSCl | Trimethylsilyl chloride |
| TMSCN | Trimethylsilyl cyanide |
| RB | Round bottom (flask) |
| RT | Room temperature |
| ca. | Circa (approximately) |
| E-VIPR | Electrical stimulation voltage ion probe reader |
| HEK | Human embryonic kidney |
| KIR2.1 | Inward-rectifier potassium ion channel 2.1 |
| DMEM | Dulbecco's Modified Eagle's Medium |
| FBS | Fetal bovine serum |
| NEAA | Non-essential amino acids |
| HEPES | 2-[4-(2-hydroxyethyl)piperazin-1-yl]ethanesulfonic acid |
| DiSBAC₆(3) | Bis-(1,3-dihexyl-thiobarbituric acid) trimethine oxonol |
| CC2-DMPE | Chlorocoumarin-2-dimyristoyl phosphatidylethanolamine |
| VABSC-1 | Voltage Assay Background Suppression Compound |
| HS | Human serum |
| BSA | Bovine Serum Albumin |

### Example 1—Preparation of Intermediates A-1 to A-20

### Intermediate A-1

### 4-benzyloxy-6-chloro-N,N-dimethyl-pyridin-2-amine

### Step 1: 4,6-dichloro-N,N-dimethyl-pyridin-2-amine

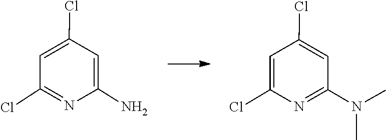

A solution of 4,6-dichloropyridin-2-amine (300 mg, 1.84 mmol) in THF (5 mL) was treated with sodium hydride (300 mg of 60% w/w, 7.50 mmol) at 0° C. Methyl iodide (732 mg, 5.16 mmol) was added and the mixture was stirred at room temperature overnight. The mixture was quenched with water and extracted with ethyl acetate. The organic layer was dried over sodium sulfate, evaporated and purified by silica gel column chromatography using 0 to 30% ethyl acetate in hexanes to give 4,6-dichloro-N,N-dimethyl-pyridin-2-amine (220 mg, 63%). ESI-MS m/z calc. 190.01, found 191.1 (M+1)⁺. ¹H NMR (400 MHz, CDCl₃) δ 6.55 (d, J=1.3 Hz, 1H), 6.34 (d, J=1.3 Hz, 1H), 3.07 (s, 6H) ppm.

### Step 2: 4-benzyloxy-6-chloro-N,N-dimethyl-pyridin-2-amine (Intermediate A-1)

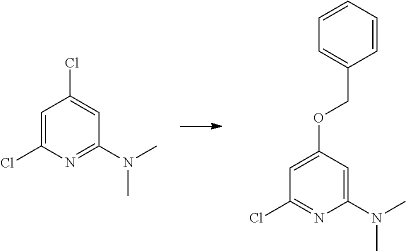 [SMILES: CN(C)c1cc(Cl)cc(Cl)n1 ; CN(C)c1cc(OCc2ccccc2)cc(Cl)n1]

To a suspension of sodium hydride (66 mg of 60% w/w, 1.65 mmol) in DMF (3 mL) was added a solution of 4,6-dichloro-N,N-dimethyl-pyridin-2-amine (220 mg, 1.15 mmol) and benzyl alcohol (137 mg, 1.27 mmol) dropwise. The mixture was stirred overnight and quenched with water. The mixture was extracted with ethyl acetate (3×15 mL), evaporated and purified by silica gel column chromatography using 0 to 10% ethyl acetate in hexanes to give 4-benzyloxy-6-chloro-N,N-dimethyl-pyridin-2-amine (Intermediate A-1, 170 mg, 53%). ESI-MS m/z calc. 262.09, found 263.3 (M+1)⁺. ¹H NMR (400 MHz, CDCl₃) δ 7.41-7.32 (m, 5H), 6.26 (d, J=1.7 Hz, 1H), 5.89 (d, J=1.8 Hz, 1H), 5.05 (s, 2H), 3.03 (s, 6H) ppm.

### Intermediate A-2

### Ethyl 4-benzyloxy-2-chloro-5,6-dimethyl-pyridine-3-carboxylate

### Step 1: ethyl 2,4-dichloro-5,6-dimethyl-pyridine-3-carboxylate

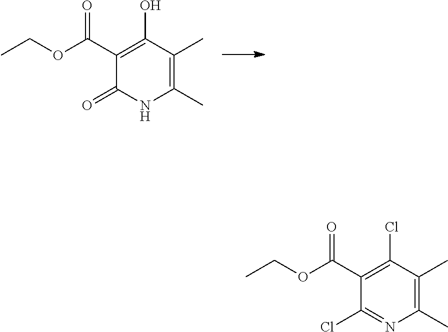

Ethyl 4-hydroxy-5,6-dimethyl-2-oxo-1H-pyridine-3-carboxylate (500 mg, 2.37 mmol) was dissolved in POCl₃ (1.5 mL, 16.1 mmol) and the solution was heated at 105° C. for 14 h. After cooling to ambient temperature, the mixture was poured onto ice and stirred for 30 min. The residue was dissolved in ethyl acetate and was carefully washed with saturated sodium bicarbonate solution (3×5 mL), filtered, and concentrated in vacuo. The crude material was purified by flash column chromatography with 0 to 30% ethyl acetate in hexanes to afford ethyl 2,4-dichloro-5,6-dimethyl-pyridine-3-carboxylate (494 mg, 83%) as a white solid. ESI-MS m/z calc. 247.02, found 248.1 (M+1)⁺.

### Step 2: ethyl 4-benzyloxy-2-chloro-5,6-dimethyl-pyridine-3-carboxylate (Intermediate A-2)

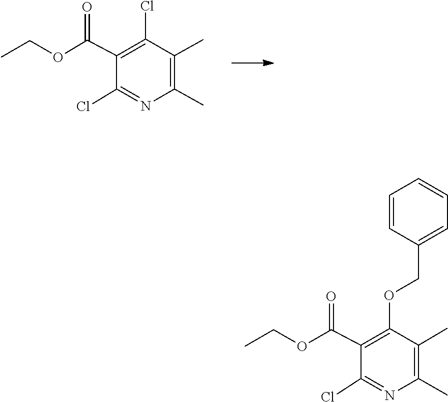 [SMILES: CCOC(=O)c1c(Cl)nc(C)c(C)c1Cl ; CCOC(=O)c1c(Cl)nc(C)c(C)c1OCc1ccccc1]

A round bottom flask equipped with a stir bar was charged with DMF (100 mL) and cooled to 0° C. Benzyl alcohol (1.160 mL, 11.21 mmol) was then added followed by sodium hydride (490 mg of 60% w/w, 12.25 mmol) and the reaction was warmed to room temperature and allowed to stir for 30 min. The reaction was then cooled to −40° C. and ethyl 2,4-dichloro-5,6-dimethyl-pyridine-3-carboxylate (3 g, 11.97 mmol) was added. The reaction was gradually warmed to room temperature and stirred overnight. The mixture was cooled to 0° C. and quenched with saturated ammonium chloride, extracted with diethyl ether (3×), washed with water, brine, dried with magnesium sulfate, filtered, and concentrated. The crude material was purified by flash column chromatography with 0 to 30% ethyl acetate in hexanes to afford ethyl 4-benzyloxy-2-chloro-5,6-dimethyl-pyridine-3-carboxylate (Intermediate A-2, 1.495 g, 41%) as an off-white solid. ESI-MS m/z calc. 319.1, found 320.0 (M+1)⁺.

### Intermediate A-3

### ethyl 4-benzyloxy-2-chloro-5-cyano-6-methyl-pyridine-3-carboxylate

### Step 1: ethyl 2-hydroxy-5-iodo-6-methyl-4-oxo-JH-pyridine-3-carboxylate

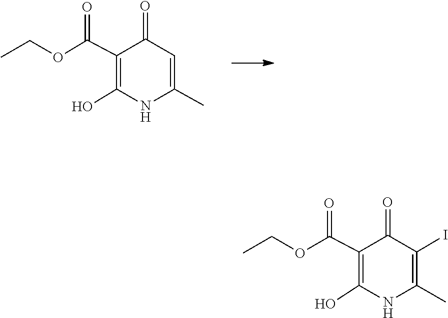

A suspension of ethyl 2-hydroxy-6-methyl-4-oxo-1H-pyridine-3-carboxylate (1 g, 5.07 mmol) and potassium carbonate (700 mg, 5.06 mmol) in water (10 mL) was heated to 100° C. Iodine (1.29 g, 5.08 mmol) was added portion wise over 10 min. After 30 min the reaction was cooled to room temperature and aqueous potassium bisulfate was added. The resulting solid was collected by suction filtration and washed with 1:1 ether/acetonitrile to give ethyl 2-hydroxy-5-iodo-6-methyl-4-oxo-TH-pyridine-3-carboxylate (1.434 g, 83%) as a white solid. ESI-MS m/z calc. 322.99, found 323.84 (M+1)⁺. ¹H NMR (400 MHz, CDCl₃) δ 4.44 (q, J=7.0 Hz, 2H), 2.57 (s, 3H), 1.42 (t, J=7.1 Hz, 3H) ppm.

### Step 2: ethyl 2,4-dichloro-5-iodo-6-methyl-pyridine-3-carboxylate

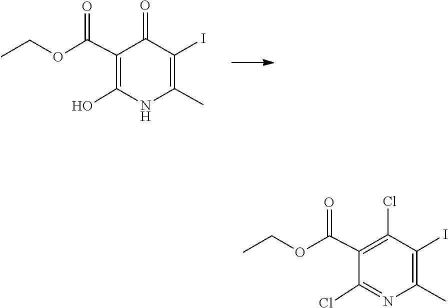

A suspension of ethyl 2-hydroxy-5-iodo-6-methyl-4-oxo-1H-pyridine-3-carboxylate (1.43 g, 4.22 mmol) in POCl₃ (16.45 g, 10 mL, 107.28 mmol) was heated at 120° C. for 2 h, concentrated and then azeotroped with toluene (2×). The residue was neutralized with a saturated aqueous solution of sodium bicarbonate and extracted with ethyl acetate (2×). The combined organics were washed with brine, dried over magnesium sulfate, filtered and concentrated under reduced pressure. The crude material was purified by silica gel flash chromatography using 0 to 10% ethyl acetate in heptane to give ethyl 2,4-dichloro-5-iodo-6-methyl-pyridine-3-carboxylate (1.15 g, 73%) as a white solid. ESI-MS m/z calc. 358.9, found 359.79 (M+1)⁺. ¹H NMR (400 MHz, CDCl₃) δ 4.46 (q, J=7.2 Hz, 2H), 2.83 (s, 3H), 1.40 (t, J=7.1 Hz, 3H) ppm.

### Step 3: ethyl 2,4-dichloro-5-cyano-6-methyl-pyridine-3-carboxylate

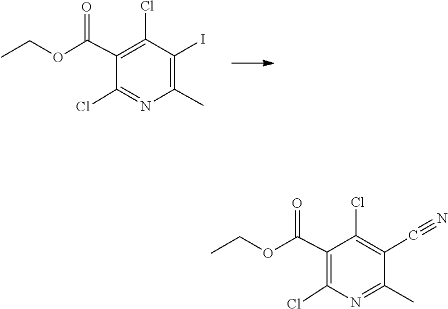 [SMILES: CCOC(=O)c1c(Cl)nc(C)c(I)c1Cl ; CCOC(=O)c1c(Cl)nc(C)c(C#N)c1Cl]

A microwave vial charged with ethyl 2,4-dichloro-5-iodo-6-methyl-pyridine-3-carboxylate (100 mg, 0.28 mmol), CuCN (28 mg, 0.3126 mmol) and NMP (1 mL) was degassed under an atmosphere of nitrogen. The vial was sealed and heated at 100° C. for 16 h. The mixture was filtered and washed with ethyl acetate. The filtrate was washed with brine (3×), dried over magnesium sulfate, filtered and concentrated. The crude material was purified via silica gel column chromatography using 0 to 10% ethyl acetate in hexanes to obtain ethyl 2,4-dichloro-5-cyano-6-methyl-pyridine-3-carboxylate (49 mg, 68%). ESI-MS m/z calc. 258.0, found 259.1 (M+1)⁺. ¹H NMR (400 MHz, DMSO-d₆) δ 4.46 (q, J=7.1 Hz, 2H), 2.71 (s, 3H), 1.34 (t, J=7.1 Hz, 3H) ppm. ¹³C NMR (101 MHz, DMSO-d₆) δ 165.17, 162.35, 149.23, 144.89, 127.34, 114.01, 110.57, 63.79, 24.26, 14.24 ppm.

### Step 4: ethyl 4-benzyloxy-2-chloro-5-cyano-6-methyl-pyridine-3-carboxylate (Intermediate A-3)

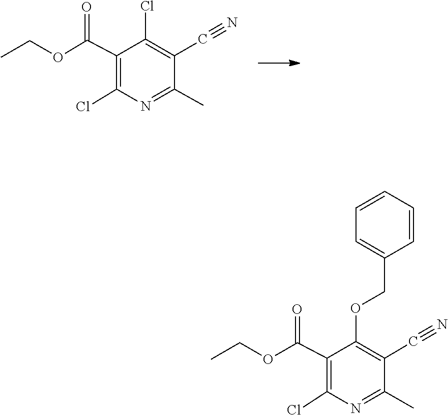 [SMILES: CCOC(=O)c1c(Cl)nc(C)c(C#N)c1Cl ; CCOC(=O)c1c(Cl)nc(C)c(C#N)c1OCc1ccccc1]

A round bottom flask equipped with a stir bar was charged with benzyl alcohol (170 μL, 1.64 mmol), THF (4 mL) and DMF (150 μL). The mixture was cooled to 0° C. and sodium hydride (70 mg of 60% w/w, 1.75 mmol) was added. The mixture was warmed to room temperature and stirred for 30 min. The mixture was again cooled to 0° C. and ethyl 2,4-dichloro-5-cyano-6-methyl-pyridine-3-carboxylate (395 mg, 1.52 mmol) was added dropwise as solution in THF (2 mL). The reaction was warmed to room temperature and stirred for 16 h. The mixture was quenched with water and the aqueous layer was extracted with ethyl acetate (3×). The combined organic layer was washed with brine, dried over magnesium sulfate, filtered and concentrated. Purification via silica gel column chromatography using 0 to 10% ethyl acetate in hexanes followed by a second purification via reverse phase column chromatography (C₁₈) using 1 to 99% acetonitrile in water containing 5 mM HCl provided ethyl 4-benzyloxy-2-chloro-5-cyano-6-methyl-pyridine-3-carboxylate (Intermediate A-3, 120 mg, 24%). ESI-MS m/z calc. 330.08, found 331.2 (M+1)⁺. ¹H NMR (400 MHz, DMSO-d₆) δ 7.51-7.35 (m, 5H), 5.47 (s, 2H), 4.33 (q, J=7.2 Hz, 3H), 2.66 (s, 3H), 1.22 (t, J=7.1 Hz, 3H) ppm.

### Intermediate A-4

### 4-(Benzyloxy)-2-bromo-3-methoxy-6-methylpyridine

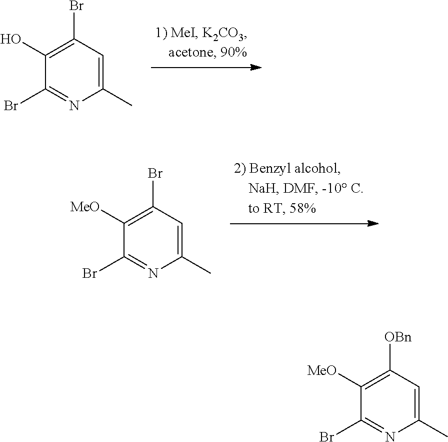

### Step 1: 2,4-dibromo-3-methoxy-6-methylpyridine

In a 2 L round bottomed flask immersed in a room temperature water bath, iodomethane (37.61 g, 16.50 mL, 265 mmol) was slowly added via syringe (no exotherm) to a stirred suspension of 2,4-dibromo-6-methylpyridin-3-ol (50 g, 178 mmol) and potassium carbonate (36.90 g, 267 mmol) in acetone (1.24 L). The suspension was stirred for 2 days at ambient temperature. The mixture was filtered, and the filter cake was washed with acetone. The brown filtrate was collected and concentrated in vacuo at 40° C. The residue was partitioned between MTBE (400 mL) and water (400 mL) and stirred at ambient temperature. The solid was filtered and rinsed with water (200 mL) to give 2,4-dibromo-3-methoxy-6-methylpyridine (49.72 g, 90%). ¹H NMR (400 MHz, DMSO-d₆) δ 7.67 (s, 1H), 3.82 (s, 3H), 2.41 (s, 3H) ppm. ESI-MS m/z calc. 280.89, found 281.8 (M+1)⁺.

### Step 2: 4-(benzyloxy)-2-bromo-3-methoxy-6-methylpyridine (Intermediate A-4)

Sodium hydride (525 mg, 60% dispersion in mineral oil, 13.08 mmol) was added to a stirred solution of benzyl alcohol (1.35 g, 12.5 mmol) in DMF (24 mL) at −10° C. and the mixture was stirred for 1 h. A solution of 2,4-dibromo-3-methoxy-6-methylpyridine (3.5 g, 12.4 mmol) in DMF (5 mL) was added. The mixture was stirred at −10° C. for 1 h then allowed to warm to ambient temperature over 30 min. The mixture was stirred at ambient temperature for an additional 1 h. The mixture was partitioned between water (75 mL) and ethyl acetate (100 mL) and the layers were separated. The organic phase was washed with water (3×75 mL) and brine, dried over magnesium sulfate, filtered and concentrated in vacuo. Purification by silica gel flash chromatography using 0 to 40% ethyl acetate in heptane gave 4-(benzyloxy)-2-bromo-3-methoxy-6-methylpyridine (Intermediate A-4, 2.25 g, 58%) as a white solid. ¹H NMR (400 MHz, CDCl₃) δ 7.42-7.33 (m, 5H), 6.69 (s, 1H), 5.14 (s, 2H), 3.85 (s, 3H), 2.43 (s, 3H) ppm. ESI-MS m/z calc. 307.02, found 308.0 (M+1)⁺.

Alternatively, 4-(benzyloxy)-2-bromo-3-methoxy-6-methylpyridine (Intermediate A-4) can be prepared via the following method:

### Step 1: 2,4-dibromo-3-methoxy-6-methyl-pyridine

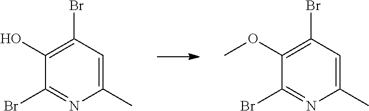

To 2,4-dibromo-6-methyl-pyridin-3-ol (1 g, 3.56 mmol) in acetone (26 mL) was added potassium carbonate (738 mg, 5.34 mmol) and iodomethane (752 mg, 0.33 mL, 5.3 mmol). The reaction was stirred at room temperature for 16 h, diluted with ethyl acetate (40 mL) and filtered. The filtrate was concentrated in vacuo. Purification by silica gel chromatography using 0-50% ethyl acetate in heptane yielded 2,4-dibromo-3-methoxy-6-methyl-pyridine (820 mg, 82%) as a yellow oil. ESI-MS m/z calc. 278.89, found 279.75 (M+1)⁺. ¹H NMR (400 MHz, CDCl₃) δ 7.30 (s, 1H), 3.89 (s, 3H), 2.47 (s, 3H) ppm.

### Step 2: 4-benzyloxy-2-bromo-3-methoxy-6-methyl-pyridine (Intermediate A-4)

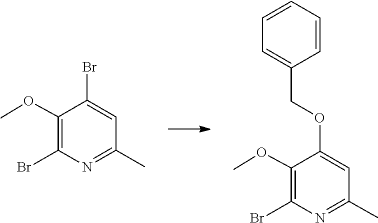

To an ice-cooled solution of benzyl alcohol (600 μL, 5.8 mmol) in 2-MeTHF (10 mL) was added sodium hydride (200 mg, 5 mmol), this mixture was allowed to stir at 5° C. for 10 min at ambient temperature after which a solution of 2,4-dibromo-3-methoxy-6-methyl-pyridine (2.569 g, 4.024 mmol) in 2-MeTHF (10 mL) was added dropwise and left to stir for a further 10 min at ambient temperature and then the mixture was heated at 80° C. for 20 h. The mixture was cooled to ambient temperature and then cooled in an ice bath. To a separate flask was added a solution of benzyl alcohol (600 μL, 5.798 mmol) in 2-MeTHF (10 mL), which was cooled to 5° C. and then sodium hydride (200 mg, 5.000 mmol) was added portionwise, after which this cooled mixture was added slowly to the main mixture at 5° C. The mixture was stirred at 5° C. for 20 min and warmed to ambient temperature. It was then heated to 80° C. for 20 h. The mixture was cooled to ambient temperature and diluted with water (10 mL). The aqueous layer was extracted with ethyl acetate (3×25 mL). The organics were washed with brine (25 mL), dried, filtered and concentrated in vacuo. Purification by silica gel chromatography using 0-100% ethyl acetate in heptane yielded 4-benzyloxy-2-bromo-3-methoxy-6-methyl-pyridine (Intermediate A-4, 1.76 g, 85%). ¹H NMR (400 MHz, CDCl₃) δ 7.40 (ddt, J=7.4, 1.3, 0.7 Hz, 2H), 7.34-7.21 (m, 3H), 6.86-6.80 (m, 1H), 5.36 (s, 2H), 3.76 (s, 3H), 2.30 (d, J=0.6 Hz, 3H) ppm. ESI-MS m/z calc. 307.02, found 308.1 (M+1)⁺.

### Intermediate A-5

### (4-benzyloxy-6-chloro-2-methyl-3-pyridyl)-imino-methyl-oxo-λ6-sulfane

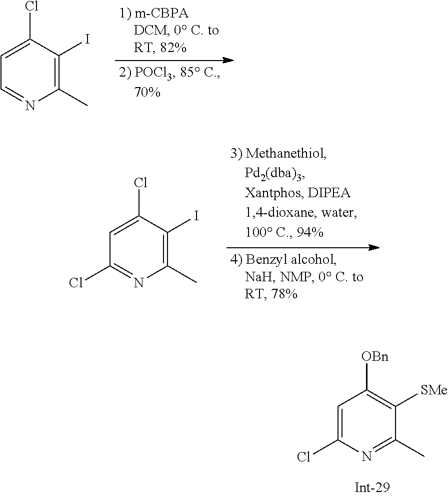

### Step 1: 4-chloro-3-iodo-2-methylpyridine 1-oxide

m-CPBA (23.4 g, 70-75% w/w in water, 102 mmol) was added portion wise to a solution of 4-chloro-3-iodo-2-methylpyridine (17.2 g, 67.86 mmol) in DCM (250 mL) at 0° C. The mixture was warmed to ambient temperature and stirred overnight. The mixture was diluted with DCM (250 mL) and washed with water (250 mL). The layers were separated and the aqueous layer was extracted with DCM (200 mL). The combined organic extracts were dried over sodium sulfate, filtered and concentrated in vacuo. The residue was triturated from methanol (2×30 mL), filtered and dried to give 4-chloro-3-iodo-2-methylpyridine 1-oxide (15 g, 82%) as a white solid. ¹H NMR (400 MHz, CDCl₃) δ 8.17 (d, J=7.3 Hz, 1H), 7.23 (d, J=6.9 Hz, 1H), 2.90 (s, 3H) ppm. ESI-MS m/z calc. 268.91, found 269.78 (M+1)⁺.

### Step 2: 4,6-dichloro-3-iodo-2-methylpyridine

A solution of 4-chloro-3-iodo-2-methylpyridine 1-oxide (1.75 g, 6.45 mmol) in POCl₃ (16.45 g, 10 mL, 107.28 mmol) was heated at 85° C. for 5 h. On cooling, the mixture was concentrated in vacuo. The residue was partitioned between ethyl acetate (50 mL) and water (20 mL). The aqueous layer was separated and extracted with ethyl acetate (2×20 mL). The combined organic extracts were dried over sodium sulfate, filtered and concentrated in vacuo to give 4,6-dichloro-3-iodo-2-methylpyridine (2.5 g, 70%) as a brown solid. ESI-MS m/z calc. 286.88, found 287.73 (M+1)⁺.

### Step 3: 4,6-dichloro-2-methyl-3-(methylthio)pyridine

A mixture of 4,6-dichloro-3-iodo-2-methylpyridine (212 mg, 0.71 mmol), sodium methanethiolate (50 mg, 0.70 mmol), Pd₂(dba)₃ (16 mg, 0.02 mmol), Xantphos (20 mg, 0.04 mmol) and DIPEA (185 mg, 250 μL, 1.42 mmol) in a mixture of 1,4-dioxane (2 mL) and water (0.2 mL) was stirred under microwave irradiation at 100° C. for 1 h. The mixture was partitioned between ethyl acetate and water. The aqueous phase was separated and extracted with ethyl acetate. The combined organic extracts were washed with brine, dried over magnesium sulfate, filtered and concentrated in vacuo. Purification by silica gel flash chromatography using 20% ethyl acetate in heptane gave 4,6-dichloro-2-methyl-3-(methylthio)pyridine (186 mg, 94%) as a pale yellow oil. ¹H NMR (400 MHz, CDCl₃) δ 7.30 (s, 1H), 2.78 (s, 3H), 2.34 (s, 3H) ppm. ESI-MS m/z calc. 206.97, found 207.94 (M+1)⁺.

### Step 4: 4-(benzyloxy)-6-chloro-2-methyl-3-(methylthio)pyridine

Benzyl alcohol (160 μL, 1.55 mmol) was added dropwise to a stirred suspension of sodium hydride (88 mg, 60% dispersion in mineral oil, 2.2 mmol) in NMP (3.0 mL) at 0° C. and the mixture stirred at ambient temperature for 15 min. A solution of 4,6-dichloro-2-methyl-3-(methylthio)pyridine (305 mg, 1.47 mmol) in NMP (3.0 mL) was added at 0° C. The mixture was warmed to ambient temperature and stirred for an additional 1 h. The mixture was quenched by addition of a saturated NH₄Cl solution (5 mL) and poured over water (50 mL). The mixture was diluted with ethyl acetate (50 mL). The aqueous layer was separated and extracted with ethyl acetate (20 mL). The combined organic extracts were washed with brine (30 mL), dried over magnesium sulfate, filtered and concentrated in vacuo. Purification by silica gel column chromatography using 0 to 30% ethyl acetate in heptane gave 4-(benzyloxy)-6-chloro-2-methyl-3-(methylthio)pyridine (319 mg, 78%) as a yellow oil. ¹H NMR (400 MHz, CDCl₃) δ 7.47-7.35 (m, 5H), 6.76 (s, 1H), 5.18 (s, 2H), 2.69 (s, 3H), 2.30 (s, 3H) ppm. ESI-MS m/z calc. 279.05, found 280.4 (M+1)⁺.

### Step 5: rac-(4-benzyloxy-6-chloro-2-methyl-3-pyridyl)-imino-methyl-oxo-λ6-sulfane (Intermediate A-5)

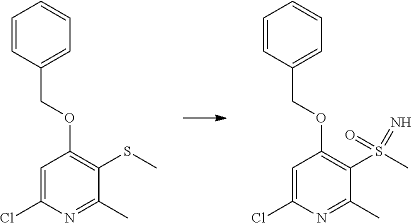 [SMILES: CSc1c(OCc2ccccc2)cc(Cl)nc1C ; Cc1nc(Cl)cc(OCc2ccccc2)c1S(C)(=N)=O]

4-benzyloxy-6-chloro-2-methyl-3-methylsulfanyl-pyridine (2.0 g, 7.15 mmol) was dissolved in DCM (20 mL) and methanol (20 mL) and cooled to 0° C. Ammonium carbamate (837 mg, 10.72 mmol) and (diacetoxyiodo)benzene (4.61 g, 14.31 mmol) were added and the mixture was allowed to warm to room temperature and stir for 2 h. Additional (diacetoxyiodo)benzene (1.15 g, 3.570 mmol) and ammonium carbamate (223 mg, 2.856 mmol) were added and the resulting mixture was left to stir at room temperature for 1.5 h. The mixture was concentrated in vacuo and purified by silica gel column chromatography using 0-100% ethyl acetate in heptane to give (4-benzyloxy-6-chloro-2-methyl-3-pyridyl)-imino-methyl-oxo-λ6-sulfane (Intermediate A-5, 1.314 g, 59%) as a white solid. ESI-MS m/z calc. 310.05, found 311.2 (M+1)⁺. ¹H NMR (500 MHz, DMSO-d₆) δ 7.56-7.54 (m, 2H), 7.45-7.40 (m, 2H), 7.39-7.35 (m, 2H), 5.39 (d, J=2.0 Hz, 2H), 4.59 (s, 1H), 3.17 (d, J=1.2 Hz, 3H), 2.74 (s, 3H) ppm.

### Step 6: (S)-(4-benzyloxy-6-chloro-2-methyl-3-pyridyl)-imino-methyl-oxo-λ6-sulfane and (R)-(4-benzyloxy-6-chloro-2-methyl-3-pyridyl)-imino-methyl-oxo-16-sulfane) (Intermediates A-5A and A-5B)

The enantiomers were separated by chiral SFC using a ChiralPak IC (250×20 mm), 5 um, (Daicel Corp.) column at 40° C. using a Nexera UC Prep (Shimadzu). Separation was achieved using an isocratic method where the mobile phase was 20% methanol (20 mM NH₃), 80% CO₂ at a flow rate of 100 mL/min. Concentration of the sample was 30 mg/mL in methanol. Injection volume was 400 uL. Outlet pressure was 100 bar. Detection wavelength was 215 nm. Retention time of Peak 1 was 4.38 minutes and Peak 2 was 5.47 minutes.
Peak 1: (4-benzyloxy-6-chloro-2-methyl-3-pyridyl)-imino-methyl-oxo-λ6-sulfane (Intermediate A-5A, 561 mg, 51%), ESI-MS m/z calc. 310.05, found 311.1 (M+1)⁺. 98.1% ee.
Peak 2: (4-benzyloxy-6-chloro-2-methyl-3-pyridyl)-imino-methyl-oxo-λ6-sulfane (Intermediate A-5B, 523 mg, 47%), ESI-MS m/z calc. 310.05, found 311.1 (M+1)⁺. 91.1% ee.

### Intermediate A-6

### 2-(4-benzyloxy-6-chloro-2-methyl-3-pyridyl)acetamide

### Step 1: 4-benzyloxy-6-chloro-3-[(E)-2-ethoxyvinyl]-2-methyl-pyridine

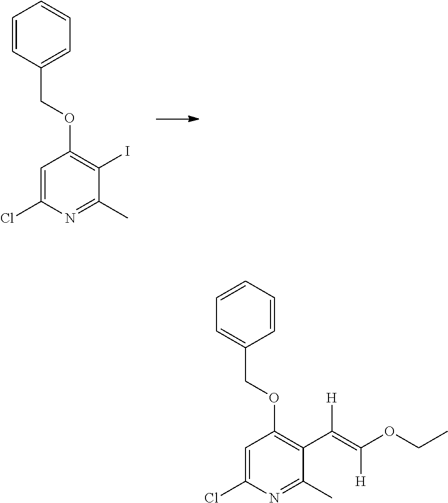 [SMILES: Cc1nc(Cl)cc(OCc2ccccc2)c1I ; CCO/C=C/c1c(OCc2ccccc2)cc(Cl)nc1C]

A microwave vial was charged with 4-benzyloxy-6-chloro-3-iodo-2-methyl-pyridine (314 mg, 0.87 mmol), 2-[(E)-2-ethoxyvinyl]-4,4,5,5-tetramethyl-1,3,2-dioxaborolane (173 mg, 0.87 mmol), PdCl₂(dtbpf) (66 mg, 0.1 mmol), potassium phosphate (550 mg, 2.6 mmol), 1,4-dioxane (3 mL) and water (1 mL). The mixture was degassed for 5 min and placed under a nitrogen atmosphere. The tube was sealed and the reaction was stirred at 110° C. for 20 h. The mixture was diluted with ethyl acetate, washed with a saturated aqueous solution of ammonium chloride and then brine. The organic layer was dried over anhydrous magnesium sulfate, filtered, and concentrated under reduced pressure. Purification by silica gel column chromatography using 0 to 50% of ethyl acetate in hexanes gave 4-benzyloxy-6-chloro-3-[(E)-2-ethoxyvinyl]-2-methyl-pyridine (188 mg, 71%). ESI-MS m/z calc. 303.10, found 304.3 (M+1)⁺. ¹H NMR (400 MHz, CDCl₃) δ 7.46-7.32 (m, 5H), 7.13 (d, J=12.7 Hz, 1H), 6.76 (s, 1H), 5.73 (d, J=12.7 Hz, 1H), 5.11 (s, 2H), 3.81 (q, J=7.0 Hz, 2H), 2.51 (s, 3H), 1.26 (t, J=7.0 Hz, 3H) ppm.

### Step 2: 2-(4-benzyloxy-6-chloro-2-methyl-3-pyridyl)acetaldehyde

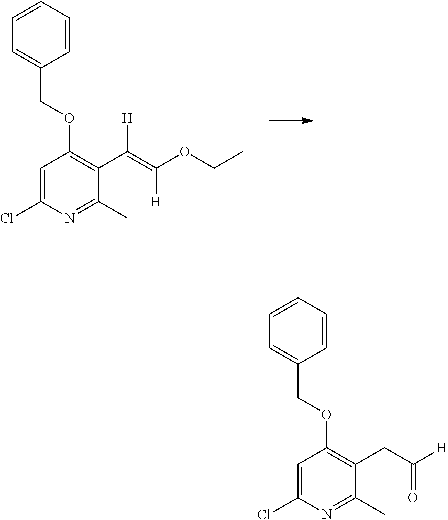 [SMILES: CCO/C=C/c1c(OCc2ccccc2)cc(Cl)nc1C ; Cc1nc(Cl)cc(OCc2ccccc2)c1CC=O]

To a solution of 4-benzyloxy-6-chloro-3-[(E)-2-ethoxyvinyl]-2-methyl-pyridine in acetone (2 mL) was added HCl (2 mL of 1 M, 2 mmol) and the mixture was stirred at 65° C. for 3 h. The mixture was diluted with ethyl acetate, washed with a saturated aqueous solution of ammonium chloride and then brine. The organic layer was dried over anhydrous magnesium sulfate, filtered, and then concentrated under reduced pressure to give 2-(4-benzyloxy-6-chloro-2-methyl-3-pyridyl)acetaldehyde.

### Step 3: 2-(4-benzyloxy-6-chloro-2-methyl-3-pyridyl)acetic acid

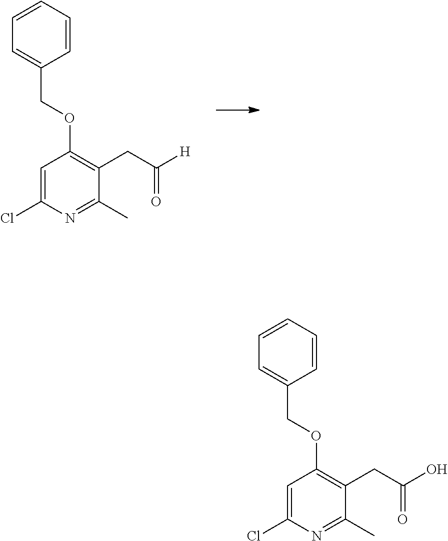 [SMILES: Cc1nc(Cl)cc(OCc2ccccc2)c1CC=O ; Cc1nc(Cl)cc(OCc2ccccc2)c1CC(=O)O]

Crude 2-(4-benzyloxy-6-chloro-2-methyl-3-pyridyl)acetaldehyde from step 2 was dissolved in DMF (2 mL), then OXONE® (545 mg, 0.89 mmol) was added and the mixture was stirred at room temperature for 1 h and the resulting mixture was diluted with ethyl acetate, washed with a saturated aqueous solution of ammonium chloride and then brine. The organic layer was dried over anhydrous magnesium sulfate, filtered, and concentrated under reduced pressure. Purification by silica gel column chromatography using 0 to 70% of ethyl acetate in heptanes gave 2-(4-benzyloxy-6-chloro-2-methyl-3-pyridyl)acetic acid (125 mg, 49%). ESI-MS m/z calc. 291.07, found 292.3 (M+1)⁺. ¹H NMR (400 MHz, DMSO-d₆) δ 12.45 (br s, 1H), 7.46-7.30 (m, 5H), 7.09 (s, 1H), 5.24 (s, 2H), 3.60 (s, 2H), 2.36 (s, 3H) ppm.

### Step 4: 2-(4-benzyloxy-6-chloro-2-methyl-3-pyridyl)acetamide (Intermediate A-6)

2-(4-benzyloxy-6-chloro-2-methyl-3-pyridyl)acetic acid (55 mg, 0.19 mmol) was dissolved in DMF (550 μL), then HATU (80 mg, 0.21 mmol), DIPEA (50 μL, 0.29 mmol), and NH₃ in methanol (150 μL of 7 M, 1.05 mmol) were added and the resulting mixture was stirred for 30 min. The resulting mixture was diluted with ethyl acetate, washed with a saturated aqueous solution of ammonium chloride and then brine. The organic layer was dried over anhydrous magnesium sulfate, filtered, and concentrated under reduced pressure. Purification by silica gel column chromatography using 0 to 100% of ethyl acetate in hexanes gave 2-(4-benzyloxy-6-chloro-2-methyl-3-pyridyl)acetamide (Intermediate A-6, 47 mg, 81%). ESI-MS m/z calc. 290.08, found 291.3 (M+1)⁺. ¹H NMR (400 MHz, DMSO-d₆) δ 7.50-7.28 (m, 6H), 7.05 (s, 1H), 6.94 (br s, 1H), 5.23 (s, 2H), 3.47 (s, 2H), 2.34 (s, 3H) ppm.

### Intermediate A-7

### 2-(4-benzyloxy-6-chloro-2-methyl-3-pyridyl)-N-methyl-acetamide

### Step 1: 2-(4-benzyloxy-6-chloro-2-methyl-3-pyridyl)-N-methyl-acetamide (Intermediate

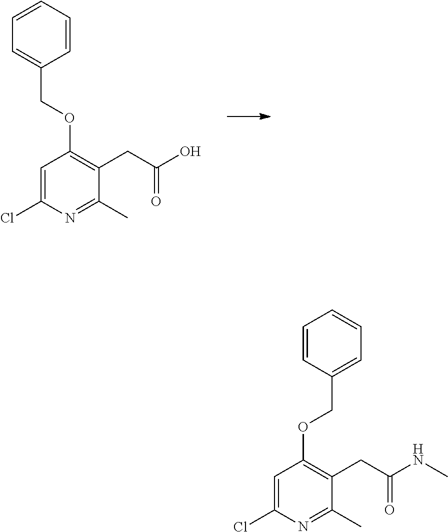 [SMILES: Cc1nc(Cl)cc(OCc2ccccc2)c1CC(=O)O ; CNC(=O)Cc1c(OCc2ccccc2)cc(Cl)nc1C]

2-(4-benzyloxy-6-chloro-2-methyl-3-pyridyl)acetic acid (73.2 mg, 0.25 mmol) (Intermediate A-6, Step 3) was dissolved in DMF (750 μL), then HATU (110 mg, 0.29 mmol), DIPEA (65 μL, 0.37 mmol), and methyl amine in EtOH (120 L of 33% w/v, 1.28 mmol) were added and the resulting mixture was stirred for 30 min. The resulting mixture was diluted with ethyl acetate, washed with a saturated aqueous solution of ammonium chloride and then brine. The organic layer was dried over anhydrous magnesium sulfate, filtered, and concentrated under reduced pressure. Purification by silica gel chromatography using 0 to 100% of ethyl acetate in hexanes gave 2-(4-benzyloxy-6-chloro-2-methyl-3-pyridyl)-N-methyl-acetamide (Intermediate A-7, 70.1 mg, 92%). ESI-MS m/z calc. 304.1, found 305.3 (M+1)⁺. ¹H NMR (400 MHz, DMSO-d₆) δ 7.79 (q, J=4.8 Hz, 1H), 7.45-7.29 (m, 5H), 7.06 (s, 1H), 5.22 (s, 2H), 3.46 (s, 2H), 2.56 (d, J=4.6 Hz, 3H), 2.34 (s, 3H) ppm.

### Intermediate A-8

### 4-benzyloxy-2-bromo-3-ethoxy-6-methyl-pyridine

### Step 1: 2,4-dibromo-3-ethoxy-6-methyl-pyridine

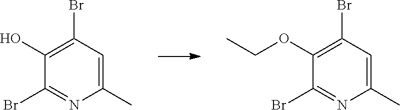

To 2,4-dibromo-6-methyl-pyridin-3-ol (7 g, 25.34 mmol) in DMF (70 mL) was added potassium carbonate (10.5 g, 75.97 mmol) and bromoethane (4.2 g, 38.54 mmol). The reaction was stirred at 70° C. for 90 min then cooled to room temperature over 30 min. The mixture was partitioned between water (200 mL) and ethyl acetate (200 mL) and the layers were separated. The organic layer was washed with water (3×200 mL), brine (200 mL), dried over magnesium sulfate, filtered and concentrated in vacuo. Purification by silica gel chromatography using 0-50% ethyl acetate in heptane yielded the product 2,4-dibromo-3-ethoxy-6-methyl-pyridine (6.65 g, 89%) as a white solid. ¹H NMR (400 MHz, CDCl₃) δ 7.29 (s, 1H), 4.08 (q, J=7.0 Hz, 2H), 2.47 (s, 3H), 1.47 (t, J=7.1 Hz, 3H) ppm. ESI-MS m/z calc. 292.91, found 293.88 (M+1)⁺.

### Step 2: 4-benzyloxy-2-bromo-3-ethoxy-6-methyl-pyridine (Intermediate A-8)

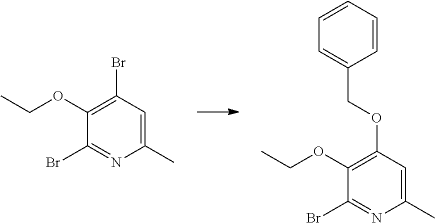 [SMILES: CCOc1c(Br)cc(C)nc1Br ; CCOc1c(OCc2ccccc2)cc(C)nc1Br]

To Benzyl alcohol (2.4 g, 22.19 mmol) in DMF (44 mL) under argon gas and at −10° C. was added sodium hydride (60% on mineral oil) (950 mg, 60% w/w, 23.68 mmol). The reaction was stirred at −10° C. for 1 h then 2,4-dibromo-3-ethoxy-6-methyl-pyridine (6.7 g, 22.71 mmol) in DMF (5 mL) was added. The reaction was stirred at −10° C. for an additional 1 h and gradually warmed to room temperature over 30 min and stirred overnight for 16 h. The mixture was partitioned between water (75 mL) and ethyl acetate (100 mL) The layers were separated and the organic layer was washed with water (3×75 mL), brine (75 mL), dried over magnesium sulfate, filtered and concentrated in vacuo. Purification by silica gel column chromatography using 0-40% ethyl acetate in heptane yielded the product 4-benzyloxy-2-bromo-3-ethoxy-6-methyl-pyridine (Intermediate A-8, 4.396 g, 60%) as a white solid. ¹H NMR (400 MHz, CDCl₃) δ 7.43-7.33 (m, 5H), 6.69 (s, 1H), 5.13 (s, 2H), 4.08 (q, J=7.0 Hz, 2H), 2.44 (s, 3H), 1.39 (t, J=7.1 Hz, 3H) ppm. ESI-MS m/z calc. 321.04, found 322.1 (M+1)⁺.

### Intermediate A-9

### 4-benzyloxy-3,6-dichloro-2-methyl-pyridine

### Step 1: 3-chloro-2-methyl-1-oxido-pyridin-1-ium

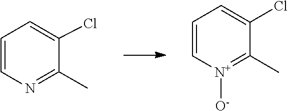

To a stirred solution of 3-chloro-2-methyl-pyridine (1 g, 7.84 mmol) in DCM (20 mL), m-CPBA (2 g, 11.6 mmol) was added at 0° C. under argon atmosphere and the mixture was stirred for 12 h. The mixture was quenched with sodium thiosulphate solution and extracted with DCM. The organic layer was washed with saturated sodium bicarbonate solution (2×), dried over anhydrous sodium sulfate and concentrated under reduced pressure to get 3-chloro-2-methyl-1-oxido-pyridin-1-ium (1 g, 89%). ¹H NMR (400 MHz, DMSO-d₆) δ 8.28 (d, J=6.8 Hz, 1H), 7.48 (d, J=8.4 Hz, 1H), 7.29 (d, J=7.2 Hz, 1H), 2.46 (s, 3H) ppm.

### Step 2: 3-chloro-2-methyl-4-nitro-1-oxido-pyridin-1-ium

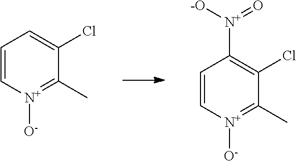

To a solution of 3-chloro-2-methyl-1-oxido-pyridin-1-ium (10 g, 69.65 mmol) in sulfuric acid (91.5 g, 50 mL, 933 mmol) at 0° C. was added drop-wise nitric acid (fuming) (45.3 g, 30 mL, 719 mmol). The solution was heated at 95° C. for 4 h. After cooling to ambient temperature, the mixture was cooled to 0° C. and treated with 10 N NaOH solution (pH˜10), diluted with excess DCM (400 mL). The layers were separated and the organic layer was evaporated under reduce pressure to afford the crude 3-chloro-2-methyl-4-nitro-1-oxido-pyridin-1-ium (8 g, 54%). ESI-MS m/z calc. 188, found 189.0 (M+1)⁺.

### Step 3: 3,4-dichloro-2-methyl-1-oxido-pyridin-1-ium

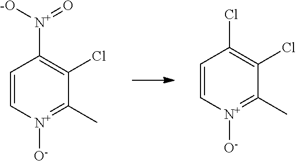

To a solution of 3-chloro-2-methyl-4-nitro-1-oxido-pyridin-1-ium (800 mg, 4.24 mmol) in EtOH (15 mL) was added acetyl chloride (883 mg, 0.8 mL, 11.25 mmol) dropwise. The solution was heated at 65° C. for 3 h. After cooling to an ambient temperature, the solvent was removed under reduced pressure, diluted with water and DCM (100 mL). Aqueous NaOH (50%) was added dropwise (pH˜10). The layers were separated and the organic layer was washed with brine solution (2×20 mL), dried over magnesium sulfate, filtered and concentrated to get 3,4-dichloro-2-methyl-1-oxido-pyridin-1-ium (700 mg, 92%). ESI-MS m/z calc. 176.97, found 178.0 (M+1)⁺.

### Step 4: 4-benzyloxy-3-chloro-2-methyl-1-oxido-pyridin-1-ium

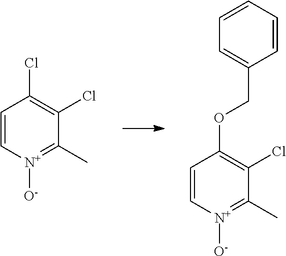

To a stirred solution of benzyl alcohol (520 mg, 0.5 mL, 4.81 mmol) in DMF (7 mL) was added sodium hydride (236 mg, 60% w/w, 5.9 mmol) at 0° C. After 10 min, 3,4-dichloro-2-methyl-1-oxido-pyridin-1-ium (700 mg, 3.93 mmol) was added and the reaction was stirred at 80° C. for 2 h. The mixture was diluted with water (20 mL) and extracted with ethyl acetate (2×30 mL). The organic layer was washed with brine (5 mL), dried over anhydrous sodium sulfate, filtered and concentrated. Purification by silica gel column chromatography using 0-10% methanol in DCM afforded 4-benzyloxy-3-chloro-2-methyl-1-oxido-pyridin-1-ium (700 mg, 66%). ESI-MS m % z calc. 249.06, found 250.0 (M+1)⁺. ¹H NMR (400 MHz, CDCl₃) δ 8.14 (d, J=7.2 Hz, 1H), 7.43-7.34 (m, 5H), 6.74 (d, J=7.3 Hz, 1H), 5.20 (s, 2H), 2.68 (s, 3H) ppm.

### Step 5: 4-benzyloxy-3,6-dichloro-2-methyl-pyridine (Intermediate A-9)

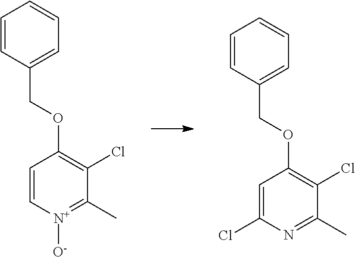 [SMILES: Cc1c(Cl)c(OCc2ccccc2)cc[n+]1[O-] ; Cc1nc(Cl)cc(OCc2ccccc2)c1Cl]

A mixture of 4-benzyloxy-3-chloro-2-methyl-1-oxido-pyridin-1-ium (1.2 g, 4.81 mmol), POCl₃ (14.8 g, 9 mL, 96.56 mmol) and pyridine (195.60 mg, 0.2 mL, 2.47 mmol) were stirred at 90° C. for 4 h. After cooling to an ambient temperature, solvent was removed under reduced pressure and the reaction was diluted with water and ethyl acetate. NaHCO₃ solution was added drop-wise (pH˜10). The layers were separated and the organic layer was washed with brine solution (2×20 mL), dried over magnesium sulfate, filtered and concentrated. Purification by silica gel column chromatography using 10-20% ethyl acetate in hexane afforded 4-benzyloxy-3,6-dichloro-2-methyl-pyridine (Intermediate A-9, 646 mg, 44%) as a white solid. ESI-MS m/z calc. 267.02, found 268.0 (M+1)⁺. ¹H NMR (400 MHz, DMSO-d₆) δ 7.50-7.33 (m, 5H), 7.32 (s, 1H), 5.33 (s, 2H), 2.48 (s, 3H) ppm.

### Intermediates A-10 to A-20

Intermediates A-10 to A-20 (see Table 1) were prepared using the appropriate dihalo-pyridine and procedure analogous to Intermediate A-1, step 2. Dichloro-pyridines were obtained from commercial sources. Benzyl alcohol or 2-methoxy benzyl alcohol can be used. DMF, THF or 2-Methyl THF can be used as the appropriate solvents.

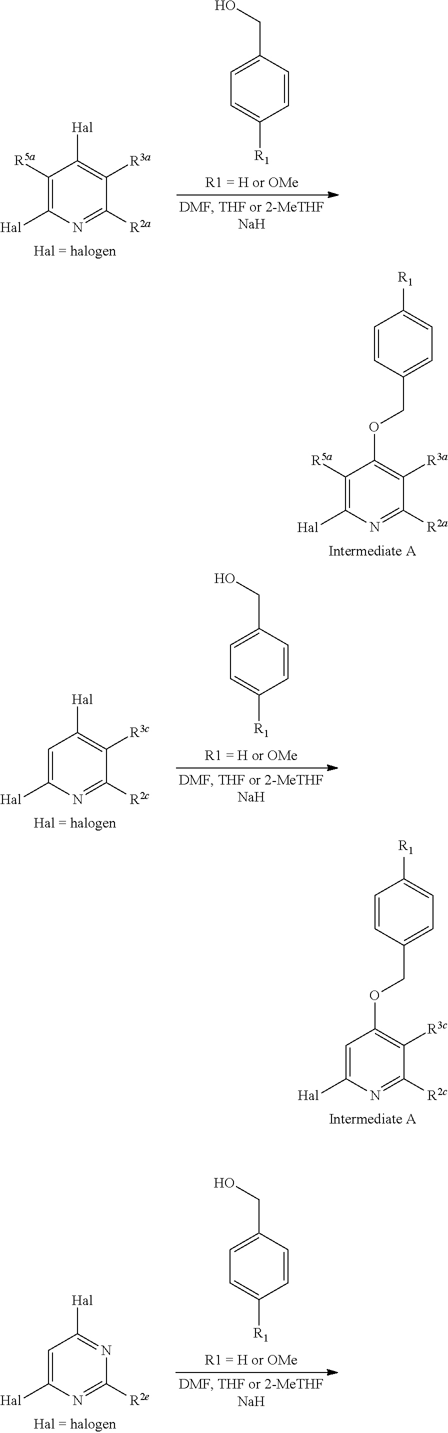

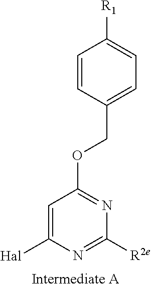

TABLE 1

| | | LC/MS | |
|---|---|---|---|
| | | (m/z calc.); | |
| | | Found | |
| Intermediate | Compound Name/Structure | M + H | NMR (shifts in ppm) |
| Intermediate A-10 | 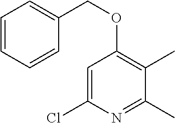 [SMILES: Cc1nc(Cl)cc(OCc2ccccc2)c1C] | 174.99 176 | ¹H NMR (400 MHz, CDCl₃) δ 7.21 (s, 1H), 2.53 (s, 3H), 2.31 (s, 3H). |
| | 4-benzyloxy-6-chloro-2,3- | | |
| | dimethyl-pyridine | | |
| Intermediate A-11 | 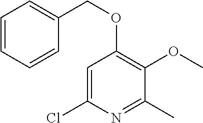 [SMILES: COc1c(OCc2ccccc2)cc(Cl)nc1C] | 263.07 264.2 | ¹H NMR (400 MHz, CDCl₃) δ 7.44-7.34 (m, 5H), 6.78 (s, 1H), 5.13 (s, 2H), 3.82 (s, 3H), 2.44 (s, 3H). |
| | 4-benzyloxy-6-chloro-3- | | |
| | methoxy-2-methyl-pyridine | | |
| Intermediate A-12 | 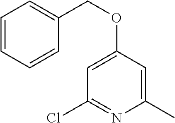 [SMILES: Cc1cc(OCc2ccccc2)cc(Cl)n1] | 233.06 234.1 | ¹H NMR (400 MHz, CDCl₃) δ 7.46-7.34 (m, 5H), 6.76 (d, J = 1.6 Hz, 1H), 6.68 (d, J = 1.6 Hz, 1H), 5.09 (s, 2H), 2.48 (s, 3H). |
| | 4-benzyloxy-2-chloro-6- | | |
| | methyl-pyridine | | |
| Intermediate A-13 | 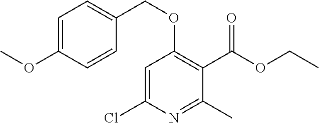 [SMILES: CCOC(=O)c1c(OCc2ccc(OC)cc2)cc(Cl)nc1C] | 335.09 336.2 | ¹H NMR (500 MHz, DMSO-d₆) δ 7.40-7.32 (m, 2H), 7.28 (s, 1H), 7.02-6.92 (m, 2H), 5.21 (s, 2H), 4.29 (q, J = 7.1 Hz, 2H), 3.76 (s, 3H), 2.35 (s, 3H), 1.21 (t, J = 7.1 Hz, 3H). |
| | ethyl 6-chloro-4-[(4- | | |
| | methoxyphenyl)methoxy]-2- | | |
| | methyl-pyridine-3-carboxylate | | |
| Intermediate A-14 | 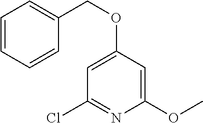 [SMILES: COc1cc(OCc2ccccc2)cc(Cl)n1] | 249.05 250.2 | ¹H NMR (400 MHz, CDCl₃) δ 7.40 (d, J = 5.8 Hz, 5H), 6.58 (d, J = 1.9 Hz, 1H), 6.19 (d, J = 1.9 Hz, 1H), 5.06 (s, 2H), 3.91 (s, 3H). |
| | 4-benzyloxy-2-chloro-6- | | |
| | methoxy-pyridine | | |
| Intermediate A-15 | 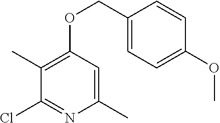 [SMILES: COc1ccc(COc2cc(C)nc(Cl)c2C)cc1] | 277.08 278.4 | ¹H NMR (500 MHz, CDCl₃) δ 7.36-7.29 (m, 2H), 6.97-6.90 (m, 2H), 6.64 (s, 1H), 5.03 (s, 2H), 3.83 (s, 3H), 2.46 (s, 3H), 22.3 (s, 3H). |
| | 2-chloro-4-[(4- | | |
| | methoxyphenyl)methoxy]-3,6- | | |
| | dimethyl-pyridine | | |
| Intermediate A-16 | 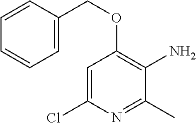 [SMILES: Cc1nc(Cl)cc(OCc2ccccc2)c1N] | 248.07 249.2 | ¹H NMR (400 MHz, DMSO-d₆) δ 7.53-7.45 (m, 2H), 7.45-7.38 (m, 2H), 7.37-7.30 (m, 1H), 6.90 (s, 1H), 5.22 (s, 2H), 4.71 (s, 2H), 2.24 (s, 3H). |
| | 4-benzyloxy-6-chloro-2- | | |
| | methyl-pyridin-3-amine | | |
| Intermediate A-17 | 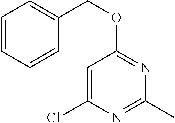 [SMILES: Cc1nc(Cl)cc(OCc2ccccc2)n1] | 234.06 235.11 | ¹H NMR (400 MHz, CDCl₃) δ 7.48-7.31 (m, 5H), 6.63 (s, 1H), 5.43 (s, 2H), 2.63 (s, 3H). |
| | 4-benzyloxy-6-chloro-2- | | |
| | methyl-pyrimidine | | |
| Intermediate A-18 | 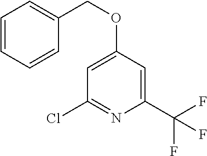 [SMILES: FC(F)(F)c1cc(OCc2ccccc2)cc(Cl)n1] | 287.03 288 | ¹H NMR (400 MHz, CDCl₃) δ 7.49-7.37 (m, 5H), 7.22 (d, J = 2.0 Hz, 1H), 7.06 (d, J = 1.7 Hz, 1H), 5.17 (s, 2H). ¹⁹F NMR (377 MHz, CDCl₃) δ −68.39 (s, 3F). |
| | 4-benzyloxy-2-chloro-6- | | |
| | (trifluoromethyl)pyridine | | |
| Intermediate A-19 | 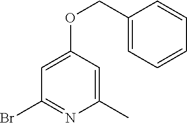 [SMILES: Cc1cc(OCc2ccccc2)cc(Br)n1] | | ¹H NMR (400 MHz, CDCl₃) δ 7.43-7.33 (m, 5H), 6.90 (s, 1H), 6.68 (s, 1H), 5.06 (s, 2H), 2.46 (s, 3H). |
| | 4-benzyloxy-2-bromo-6- | | |
| | methyl-pyridine | | |
| Intermediate A-20 | 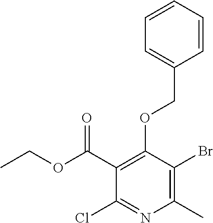 [SMILES: CCOC(=O)c1c(Cl)nc(C)c(Br)c1OCc1ccccc1] | 430.98 432 | ¹H NMR (400 MHz, DMSO-d₆) δ 7.53-7.32 (m, 5H), 5.10 (s, 2H), 4.35 (q, J = 7.1 Hz, 2H), 2.71 (s, 3H), 1.26 (t, J = 7.1 Hz, 3H). |
| | ethyl 4-(benzyloxy)-5- | | |
| | bromo-2-chloro-6- | | |
| | methylnicotinate | | |

### Intermediate A-21

### 4-benzyloxy-6-chloro-N,2-trimethyl-pyridin-3-amine

### Step 1: 4-benzyloxy-6-chloro-N,N,2-trimethyl-pyridin-3-amine (Intermediate A-20)

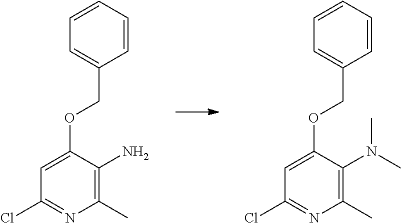 [SMILES: Cc1nc(Cl)cc(OCc2ccccc2)c1N ; Cc1nc(Cl)cc(OCc2ccccc2)c1N(C)C]

In a 1-dram vial, 4-benzyloxy-6-chloro-2-methyl-pyridin-3-amine (hydrochloride salt) (Intermediate A-16, 130.2 mg, 0.46 mmol) was mixed with AcOH (500 μL), to which aqueous formaldehyde (250 μL, 9.07 mmol) was added. Then, sodium cyanoborohydride (110.5 mg, 1.758 mmol) was added portion wise over 3 min. The resulting white slurry was stirred vigorously at room temperature under air for 2.5 h. It was then diluted with methanol (2 mL), filtered, and purified by reverse-phase HPLC (C18, 1-70% acetonitrile/5 mM HCl) to give 4-benzyloxy-6-chloro-N,N,2-trimethyl-pyridin-3-amine (hydrochloride salt) (Intermediate A-20, 86.0 mg, 45%). ESI-MS m/z calc. 276.10, found 277.2 (M+1)⁺.

### Intermediate A-22

### 4-benzyloxy-6-chloro-3-cyclopropyl-2-methyl-pyridine

### Step 1: 4-benzyloxy-3-bromo-6-chloro-2-methyl-pyridine

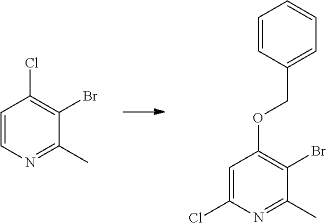

4-benzyloxy-3-bromo-6-chloro-2-methyl-pyridine was prepared from 3-bromo-4-chloro-2-methyl-pyridine using procedure analogous to that found in Intermediate A-9 (Step 1 to Step 5). ESI-MS m/z calc. 310.97, found 314.0 (M+3)⁺. ¹H NMR (400 MHz, DMSO-d₆) δ 7.52-7.46 (m, 2H), 7.46-7.41 (m, 2H), 7.40-7.35 (m, 1H), 7.23 (s, 1H), 5.35 (s, 2H), 2.54 (s, 3H).

### Step 2: 4-benzyloxy-6-chloro-3-cyclopropyl-2-methyl-pyridine (Intermediate A-22)

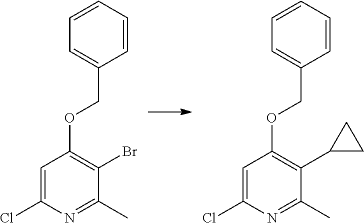 [SMILES: Cc1nc(Cl)cc(OCc2ccccc2)c1Br ; Cc1nc(Cl)cc(OCc2ccccc2)c1C1CC1]

A microwave vial charged with from 4-benzyloxy-3-bromo-6-chloro-2-methyl-pyridine (200 mg, 0.64 mmol), cyclopropylboronic acid (60 mg, 0.7 mmol), Pd(dppf)Cl₂·DCM (30 mg, 0.4 mmol), K₂CO₃ (800 μL of 2 M, 1.600 mmol), and 1,2-Dimethoxyethane (2 mL) was sealed and subjected to microwave irradiation at 100° C. for 16 hours. The reaction mixture was filtered and the solvent was evaporated. The crude material was purified by reverse phase preparative chromatography (C₁₈) using 40 to 70% acetonitrile in water containing 5 mM hydrochloric acid to give 4-benzyloxy-6-chloro-3-cyclopropyl-2-methyl-pyridine (Intermediate A-22, 75.1 mg, 41%). ¹H NMR (400 MHz, DMSO-d6) δ 7.48 (d, 2H), 7.43 (t, 2H), 7.39-7.32 (m, 1H), 7.04 (s, 1H), 5.20 (s, 2H), 2.48 (s, 3H), 1.65-1.53 (m, 1H), 0.98-0.85 (m, 2H), 0.68-0.56 (m, 2H). ESI-MS m/z calc. 273.09, found 274.0 (M+1)⁺.

### Intermediate A-23

### (4-(benzyloxy)-6-chloro-2-methylpyridin-3-yl)(imino)(isopropyl)-16-sulfanone

### Step 1: 4-benzyloxy-6-chloro-3-isopropylsulfanyl-2-methyl-pyridine

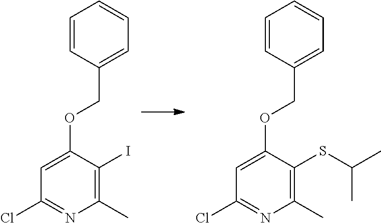 [SMILES: Cc1nc(Cl)cc(OCc2ccccc2)c1I ; Cc1nc(Cl)cc(OCc2ccccc2)c1SC(C)C]

To a degassed solution of 4-benzyloxy-6-chloro-3-iodo-2-methyl-pyridine (850 mg, 2.34 mmol) and propane-2-thiol (Sodium Ion (1)) (464 mg, 4.68 mmol) in dioxane (23 mL) was added XantPhos (135 mg, 0.23 mmol), Pd₂(dba)₃ (107 mg, 0.12 mmol) and DIPEA (890 mg, 1.2 mL, 6.9 mmol). The reaction mixture was stirred at 100° C. for 2 h. The reaction mixture was cooled to room temperature and quenched with saturated ammonium chloride solution (30 mL) and water (30 mL) and extracted with EtOAc (2×40 mL). The combined extracts were washed with water (40 mL) and brine (40 mL), dried over sodium sulfate, filtered and concentrated. The crude was purified by silica gel column chromatography using 5 to 10% EtOAc in hexanes to give 4-benzyloxy-6-chloro-3-isopropylsulfanyl-2-methyl-pyridine (680 mg, 90%). EST-MS m/z calc. 307.07, found 308.03 (M+1)⁺. ¹H-NMR (400 MHz, CDCl₃) δ 7.48-7.33 (5H, m), 6.76 (s, 1H), 5.16 (s, 2H), 3.42-3.35 (m, 1H), 2.68 (s, 3H), 1.18 (d, J=6.9 Hz, 6H).

### Step 2: (4-(benzyloxy)-6-chloro-2-methylpyridin-3-yl)(imino)(isopropyl)-16-sulfanone (Intermediate A-23)

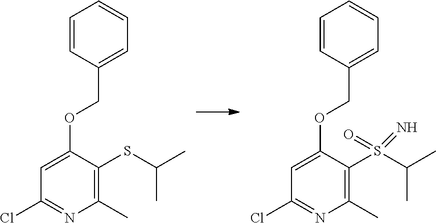 [SMILES: Cc1nc(Cl)cc(OCc2ccccc2)c1SC(C)C ; Cc1nc(Cl)cc(OCc2ccccc2)c1S(=N)(=O)C(C)C]

(4-(benzyloxy)-6-chloro-2-methylpyridin-3-yl)(imino)(isopropyl)-16-sulfanone (Intermediate A-23) was prepared from 4-benzyloxy-6-chloro-3-isopropylsulfanyl-2-methyl-pyridine, using a procedure analogous to that found in Intermediate A-5 (Step 5). ESI-MS m/z calc. 338.08, found 339.07 (M+1)⁺. ¹H-NMR (400 MHz, DMSO-d₆) δ 7.52 (d, J=7.6 Hz, 2H), 7.42-7.30 (m, 4H), 5.35 (J=12.2 Hz, 2H), 4.48 (br s, 1H), 3.62-3.55 (m, 1H), 2.70 (s, 3H), 1.10 (dd, J=45.4, 6.5 Hz, 6H).

### Intermediate A-24

### 5-(4-benzyloxy-6-chloro-2-methyl-3-pyridyl)oxazolidin-2-one

### Step 1: 4-benzyloxy-6-chloro-2-methyl-pyridine-3-carboxylic acid

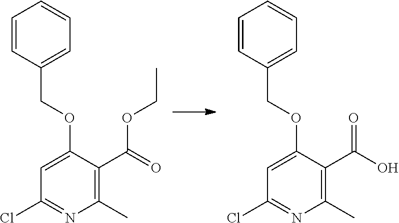 [SMILES: CCOC(=O)c1c(OCc2ccccc2)cc(Cl)nc1C ; Cc1nc(Cl)cc(OCc2ccccc2)c1C(=O)O]

To a solution of ethyl 4-benzyloxy-6-chloro-2-methyl-pyridine-3-carboxylate (1 g, 3.24 mmol) in THF (10 mL) and methanol (10 mL) was added a solution of lithium hydroxide (700 mg, 16.68 mmol) in water (10 mL) at room temperature and the mixture was stirred for 2 h at 50° C. Another portion of lithium hydroxide (700 mg, 16.681 mmol) in water (10 mL) was added at room temperature and the mixture was stirred at 50° C. for 4 days. The reaction mixture was cooled to room temperature, concentrated under reduced pressure, the residue was dissolved in water (20 mL) and acidified with a 1 M aqueous solution of hydrochloric acid until pH˜3/4. The precipitate was then filtered, rinsed with water (50 mL) and dried under high vacuum to obtain 4-benzyloxy-6-chloro-2-methyl-pyridine-3-carboxylic acid (1 g, 104%) as a white solid. ¹H NMR (400 MHz, DMSO-d₆) δ 13.49 (br. s, 1H), 7.44-7.32 (m, 5H), 7.23 (s, 1H), 5.29 (s, 2H), 2.37 (s, 3H). ESI-MS m/z calc. 277.05, found 278.2 (M+1)⁺.

### Step 2: 1-(4-benzyloxy-6-chloro-2-methyl-3-pyridyl)-2-chloro-ethanone

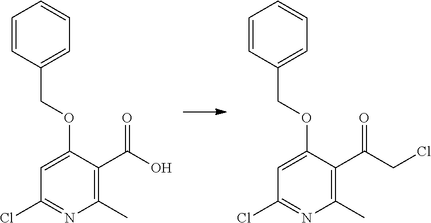 [SMILES: Cc1nc(Cl)cc(OCc2ccccc2)c1C(=O)O ; Cc1nc(Cl)cc(OCc2ccccc2)c1C(=O)CCl]

To a stirring solution of 4-benzyloxy-6-chloro-2-methyl-pyridine-3-carboxylic acid (860 mg, 2.9 mmol) and DMF (19 mg, 20 μL, 0.26 mmol) in dichloromethane (7 mL) at 0° C. was added oxalyl chloride (1.45 g, 1 mL, 11.46 mmol) dropwise. The reaction was allowed to warm to room temperature and stirred for 18 h. The mixture was concentrated and the residue then taken up into THF (7 mL) and cooled to 0° C., (trimethylsilyl)diazomethane (in hexanes) (4.3 mL of 2 M, 8.6 mmol) was added dropwise. The reaction mixture was gradually warmed to room temperature and stirred for 20 h. Additional (trimethylsilyl)diazomethane (in hexanes) (1 mL of 2 M, 2 mmol) was added at 0° C. and the reaction was warmed to room temperature and stirred for 18 h. It was concentrated and the residue was re-dissolved in THF (7 mL), cooled to 0° C. and hydrochloric acid in dioxane (1 mL of 4 M, 4 mmol) was added dropwise. The reaction mixture was warmed to room temperature and stirred for 1 h. The reaction mixture was concentrated, and the residue partitioned between ethyl acetate (50 mL) and saturated sodium hydrogen carbonate (50 mL). The organic phase was washed with brine (50 mL), dried over magnesium sulfate, filtered and concentrated. Purification by silica gel chromatography using 0 to 20% ethyl acetate in heptane gave 1-(4-benzyloxy-6-chloro-2-methyl-3-pyridyl)-2-chloro-ethanone (396 mg, 42%) as an off-white solid. ESI-MS m/z calc. 309.03, found 309.97 (M+1)⁺. ¹H-NMR (400 MHz, CDCl₃) δ 7.46-7.36 (m, 5H), 6.86 (s, 1H), 0.15 (s, 2H), 4.38 (s, 2H), 2.46 (s, 3H).

### Step 3: 2-[2-(4-benzyloxy-6-chloro-2-methyl-3-pyridyl)-2-oxo-ethyl]isoindoline-1,3-dione

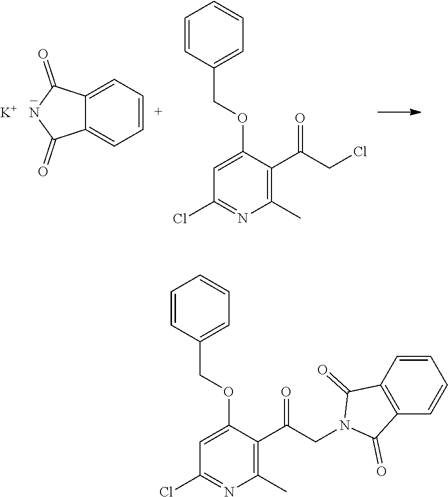 [SMILES: [K+] ; O=C1NC(=O)c2ccccc21 ; Cc1nc(Cl)cc(OCc2ccccc2)c1C(=O)CCl ; Cc1nc(Cl)cc(OCc2ccccc2)c1C(=O)CN1C(=O)c2ccccc2C1=O]

A mixture of 1-(4-benzyloxy-6-chloro-2-methyl-3-pyridyl)-2-chloro-ethanone (9.18 g, 28.15 mmol) and phthalimide potassium salt (5.75 g, 31 mmol) in DMF (200 mL) was stirred for 48 hours at room temperature. The reaction mixture was partitioned between ethyl acetate (800 mL) and water (800 mL). The organic phase was washed with water (2×600 mL), then brine (350 mL). The organic phase was dried over magnesium sulphate. Filtration and removal of the solvent under reduced pressure gave the crude product that was triturated using diethyl ether (2×100 mL). The resulting precipitate was isolated by filtration and dried under reduced pressure (50° C.) to give 2-[2-(4-benzyloxy-6-chloro-2-methyl-3-pyridyl)-2-oxo-ethyl]isoindoline-1,3-dione (9.77 g, 81%) as a white solid. ESI-MS m/z calc. 420.08, found 421. (M+1)⁺. ¹H-NMR (400 MHz, CDCl₃) δ 7.92-7.83 (m, 2H), 7.80-7.69 (m, 2H), 7.47-7.36 (m, 5H), 6.85 (s, 1H), 5.23 (s, 2H), 4.84 (s, 2H), 2.50 (s, 3H).

### Step 4: 2-[2-(4-benzyloxy-6-chloro-2-methyl-3-pyridyl)-2-hydroxy-ethyl]isoindoline-1,3-dione

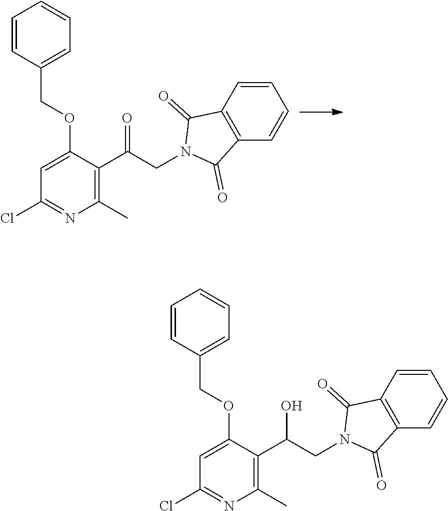 [SMILES: Cc1nc(Cl)cc(OCc2ccccc2)c1C(=O)CN1C(=O)c2ccccc2C1=O ; Cc1nc(Cl)cc(OCc2ccccc2)c1C(O)CN1C(=O)c2ccccc2C1=O]

To a solution of 2-[2-(4-benzyloxy-6-chloro-2-methyl-3-pyridyl)-2-oxo-ethyl]isoindoline-1,3-dione (50 mg, 0.11 mmol) in acetic acid (5 mL) was added sodium cyanoborohydride (14 mg, 0.22 mmol). The mixture was stirred at room temperature for 2 hours. Additional sodium cyanoborohydride (164 mg, 2.62 mmol) was added and the mixture stirred for 3 days. The reaction was quenched using saturated sodium bicarbonate solution (50 mL) and extracted with ethyl acetate (50 mL). The organic phase was washed with brine (50 mL), dried over magnesium sulfate, filtered and concentrated. Purification by reverse phase chromatography (C₁₈) using 20 to 80% acetonitrile in water containing 0.1% formic acid gave 2-[2-(4-benzyloxy-6-chloro-2-methyl-3-pyridyl)-2-hydroxy-ethyl]isoindoline-1,3-dione (30 mg, 61%) as a white solid. ESI-MS m/z calc. 422.10, found 423.13 (M+1)⁺. ¹H-NMR (400 MHz, CDCl₃) δ 7.83-7.80 (m, 2H), 7.74-7.69 (m, 2H), 7.54-7.40 (m, 5H), 6.86 (s, 1H), 5.35-5.14 (m, 3H), 4.28-4.15 (m, 1H), 3.91 (dd, J=13.7, 5.3 Hz, 1H), 3.35 (d, J=11.4 Hz, 1H), 2.54 (s, 3H).

### Step 5: 2-amino-1-(4-benzyloxy-6-chloro-2-methyl-3-pyridyl)ethanol

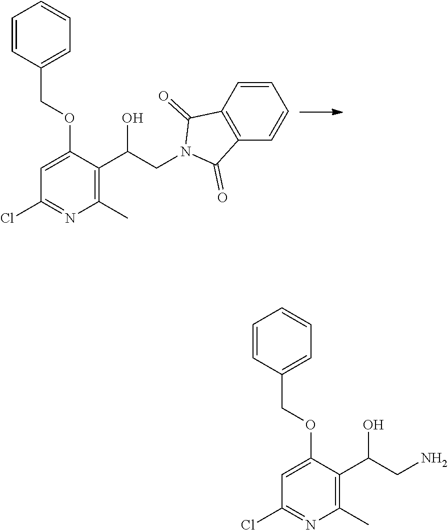 [SMILES: Cc1nc(Cl)cc(OCc2ccccc2)c1C(O)CN1C(=O)c2ccccc2C1=O ; Cc1nc(Cl)cc(OCc2ccccc2)c1C(O)CN]

To a solution of 2-[2-(4-benzyloxy-6-chloro-2-methyl-3-pyridyl)-2-hydroxy-ethyl]isoindoline-1,3-dione (2.47 g, 5.45 mmol) in ethanol (100 mL) was added hydrazine hydrate (2.73 g, 2.65 mL, 54.5 mmol). The mixture was stirred at room temperature over the weekend. The precipitate was removed by filtration and the residue concentrated under reduced pressure. The crude product was purified by reverse phase chromatography (C₁₈) using 20 to 70% acetonitrile in water (0.1% ammonia) to give 2-amino-1-(4-benzyloxy-6-chloro-2-methyl-3-pyridyl)ethanol (1.65 g, 87%). ESI-MS m/z calc. 292.09, found 293.05 (M+1)⁺. ¹H-NMR (400 MHz, CD₃OD) δ 7.51-7.31 (m, 5H), 7.01 (s, 1H), 5.22-5.17 (m, 2H), 5.16-5.11 (m, 1H), 3.13-3.00 (m, 1H), 2.80-2.70 (m, 1H), 2.56 (s, 3H). (OH and NH₂ exchangeable protons not observed.)

### Step 6: 5-(4-benzyloxy-6-chloro-2-methyl-3-pyridyl)oxazolidin-2-one (Intermediate A-24)

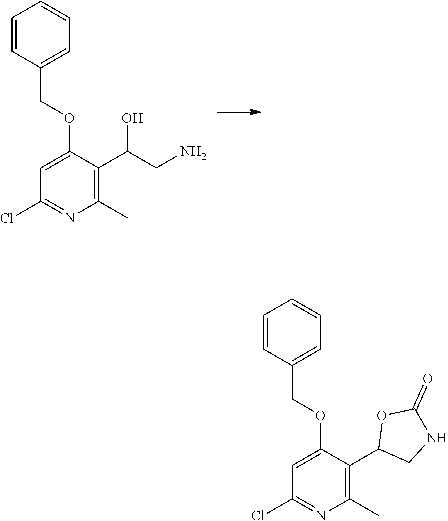 [SMILES: Cc1nc(Cl)cc(OCc2ccccc2)c1C(O)CN ; Cc1nc(Cl)cc(OCc2ccccc2)c1C1CNC(=O)O1]

To a solution of 2-amino-1-(4-benzyloxy-6-chloro-2-methyl-3-pyridyl)ethanol (1.55 g, 4.43 mmol) in DCM (25 mL) was added CDI (755 mg, 4.65 mmol) and the mixture stirred at room temperature overnight. The organic phase was partitioned between DCM (250 mL) and water (100 mL). The organic phase was washed with brine (50 mL), dried over magnesium sulfate, filtered and concentrated. The crude was purified by silica gel column chromatography using 0 to 3% MeOH in DCM to give 5-(4-benzyloxy-6-chloro-2-methyl-3-pyridyl)oxazolidin-2-one (Intermediate A-24, 762 mg, 53%) as a white solid. ESI-MS m/z calc. 318.07, found 319.03 (M+1)⁺. ¹H-NMR (400 MHz, DMSO-d₆) δ 7.61 (s, 1H), 7.44-7.42 (m, 2H), 7.38-7.28 (m, 3H), 7.18 (s, 1H), 5.89 (t, J=9.2 Hz, 1H), 5.31-5.21 (m, 2H), 3.72 (t, J=8.9 Hz, 1H), 3.48-3.43 (m, 1H), 2.41 (s, 3H).

### Intermediate A-25

### 6-chloro-4-[(4-methoxyphenyl)methoxy]-2-methyl-pyridine-3-carbonitrile

### Step 1: 6-chloro-4-[(4-methoxyphenyl)methoxy]-2-methyl-pyridine-3-carboxamide

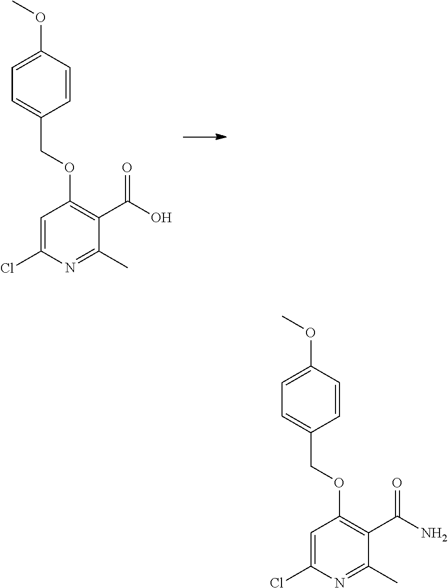 [SMILES: COc1ccc(COc2cc(Cl)nc(C)c2C(=O)O)cc1 ; COc1ccc(COc2cc(Cl)nc(C)c2C(N)=O)cc1]

To a solution of 6-chloro-4-[(4-methoxyphenyl)methoxy]-2-methyl-pyridine-3-carboxylic acid (1 g, 3.2496 mmol) in DCM (30 mL) and DMF (5 mL) was added triethylamine (1.1 g, 1.5 mL, 10.76 mmol) followed by HATU (1.9 g, 5 mmol) and ammonium chloride (550 mg, 10.28 mmol) and the reaction mixture was stirred at room temperature for 16 h. The reaction mixture was quenched with water (80 mL) and extracted with DCM (2×150 mL). The combined organic layer was washed with brine (80 mL), dried over anhydrous sodium sulfate, filtered and concentrated in vacuo to get crude 6-chloro-4-[(4-methoxyphenyl)methoxy]-2-methyl-pyridine-3-carboxamide (1.4 g, 89%). ESI-MS m/z calc. 306.07, found 307.16 (M+1)⁺.

### Step 2: 6-chloro-4-[(4-methoxyphenyl)methoxy]-2-methyl-pyridine-3-carbonitrile (Intermediate A-25)

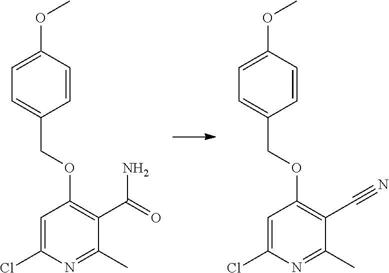 [SMILES: COc1ccc(COc2cc(Cl)nc(C)c2C(N)=O)cc1 ; COc1ccc(COc2cc(Cl)nc(C)c2C#N)cc1]

To a stirred solution of 6-chloro-4-[(4-methoxyphenyl)methoxy]-2-methyl-pyridine-3-carboxamide (4 g, 13.04 mmol) in DCM (30 mL), TEA (4 g, 5.5 mL, 39.46 mmol) was added and stirred at 0° C. for 30 min. Then trichloro acetylchloride (4.86 g, 3 mL, 26.73 mmol) was added at 0° C. and the reaction mixture was stirred at room temperature for 12 h. It was diluted with water (50 ml) and extracted with DCM (2×20 ml). The organic layer was washed with brine (20 ml), dried over sodium sulfate, filtered and concentrated under reduced pressure. Purification by silica gel column chromatography using 10 to 15% ethyl acetate in hexane gave 6-chloro-4-[(4-methoxyphenyl)methoxy]-2-methyl-pyridine-3-carbonitrile (Intermediate A-25, 2.1 g, 55%). ¹H NMR (400 MHz, CDCl₃) δ 7.38-7.29 (m, 2H), 6.97-6.89 (m, 2H), 6.83 (s, 1H), 5.16 (s, 2H), 3.82 (s, 3H), 2.67 (s, 3H).

### Intermediate A-26

### 2-(4-benzyloxy-6-chloro-2-methyl-3-pyridyl)-1,2-thiazolidine 1,1-dioxide

### Step 1: N-(4-benzyloxy-6-chloro-2-methyl-3-pyridyl)-3-chloro-propane-1-sulfonamide

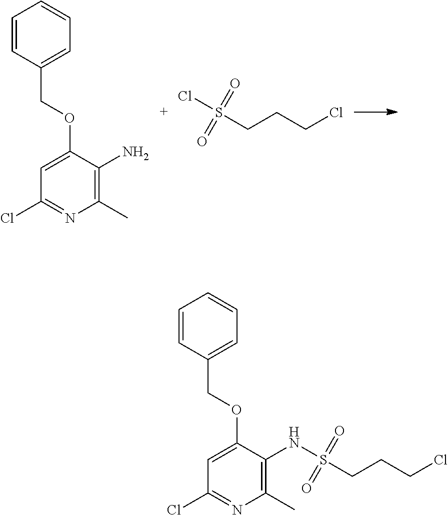 [SMILES: Cc1nc(Cl)cc(OCc2ccccc2)c1N ; O=S(=O)(Cl)CCCCl ; Cc1nc(Cl)cc(OCc2ccccc2)c1NS(=O)(=O)CCCCl]

To a solution of 4-benzyloxy-6-chloro-2-methyl-pyridin-3-amine (365 mg, 1.40 mmol) in DCM (14 mL) at 0° C. was added DIPEA (890 mg, 1.2 mL, 6.89 mmol), followed by 3-chloropropane-1-sulfonyl chloride (291 mg, 200 μL, 1.64 mmol). Stirring was continued for 1 h at 0° C., when a further portion of 3-chloropropane-1-sulfonyl chloride (291 mg, 200 μL, 1.64 mmol) was added, followed 1 h later with another portion 3-chloropropane-1-sulfonyl chloride (291 mg, 200 μL, 1.64 mmol). After another 1 h, the reaction was quenched by addition of saturated aqueous ammonium chloride (20 mL) and water (40 mL). The reaction mixture was extracted with DCM (2×40 mL), washed with water (40 mL) and brine (40 mL), dried over sodium sulfate and concentrated in vacuo. It was taken up in dioxane (14 mL) and to the stirring mixture was added aqueous NaOH (700 L of 2 M, 1.4 mmol). After 1 h, the reaction was quenched by addition of saturated aqueous ammonium chloride (20 mL) and water (40 mL). The reaction mixture was extracted with DCM (2×40 mL), washed with water (40 mL) and brine (40 mL), dried over sodium sulfate and concentrated in vacuo to give N-(4-benzyloxy-6-chloro-2-methyl-3-pyridyl)-3-chloro-propane-1-sulfonamide (848 mg, 100%) ESI-MS m/z calc. 388.04, found 389.07 (M+1)⁺.

### Step 2: 2-(4-benzyloxy-6-chloro-2-methyl-3-pyridyl)-1,2-thiazolidine 1,1-dioxide (Intermediate A-26)

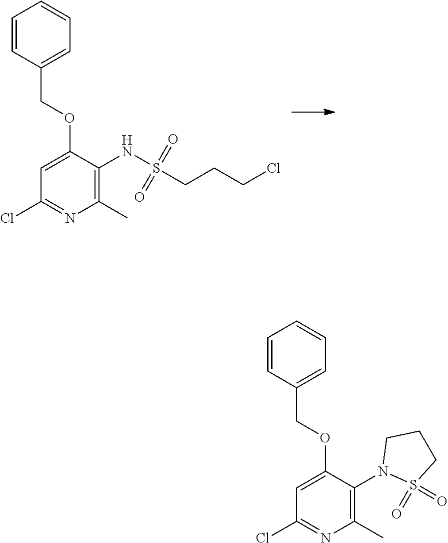 [SMILES: Cc1nc(Cl)cc(OCc2ccccc2)c1NS(=O)(=O)CCCCl ; Cc1nc(Cl)cc(OCc2ccccc2)c1N1CCCS1(=O)=O]

To crude N-(4-benzyloxy-6-chloro-2-methyl-3-pyridyl)-3-chloro-propane-1-sulfonamide (848 mg, 1.40 mmol) in DMF (14 mL) at 0° C. under argon was added NaH in oil (168 mg, 60% w/w, 4.2 mmol). The resulting mixture was warmed to RT over 1 h. The mixture was then heated at 50° C. for 1 h. On cooling, the reaction mixture was quenched by addition of saturated ammonium chloride solution (30 mL). It was then diluted with water (20 mL) and extracted with EtOAc (2×35 mL). The combined organic layer was washed with water (35 mL), brine (35 mL), dried over sodium sulfate, filtered and concentrated. The crude was purified by two successive purifications using silica gel column chromatography using 40 to 80% EtOAc in heptane to give 2-(4-benzyloxy-6-chloro-2-methyl-3-pyridyl)-1,2-thiazolidine 1,1-dioxide (Intermediate A-26, 129 mg, 24%). ESI-MS m/z calc. 352.06, found 353.09 (M+1)⁺. ¹H-NMR (400 MHz, CDCl₃) δ 7.47-7.39 (m, 5H), 6.85 (s, 1H), 5.09 (ABq, J=10.8 Hz, 2H), 3.58-3.55 (m, 2H), 3.11 (dq, J=11.8, 3.4 Hz, 1H), 2.80 (dt, J=11.6, 7.9 Hz, 1H), 2.58 (s, 3H), 2.53-2.41 (m, 1H), 2.32-2.26 (m, 1H).

### Intermediate A-27

### 2-(4-benzyloxy-6-chloro-2-methyl-3-pyridyl)-5-methyl-1,3,4-oxadiazole

### Step 1: tert-butyl N-[(4-benzyloxy-6-chloro-2-methyl-pyridine-3-carbonyl)amino]carbamate

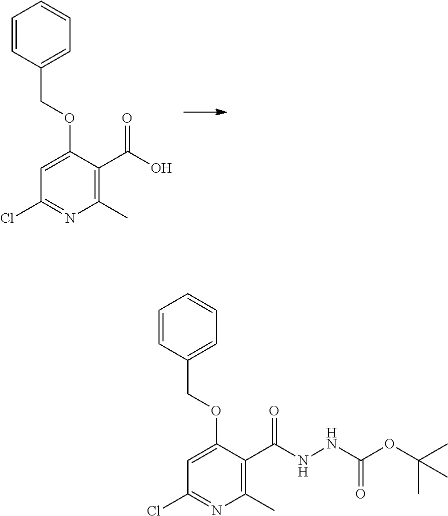 [SMILES: Cc1nc(Cl)cc(OCc2ccccc2)c1C(=O)O ; Cc1nc(Cl)cc(OCc2ccccc2)c1C(=O)NNC(=O)OC(C)(C)C]

To a stirring solution of 4-benzyloxy-6-chloro-2-methyl-pyridine-3-carboxylic acid (1 g, 2.85 mmol) and DMF (95 mg, 100 μL, 1.3 mmol) in dichloromethane (10 mL) at 0° C. was added oxalyl chloride (1.45 g, 1 mL, 11.46 mmol) dropwise. The mixture was allowed to warm to room temperature and stirred for 18 h. The reaction was concentrated, then re-dissolved in dichloromethane (10 mL) and added dropwise to a solution of Hunig's base (1.11 g, 1.5 mL, 8.61 mmol) and tert-butyl carbazate (420 mg, 3.18 mmol) in dichloromethane (10 mL) at 0° C. The reaction was warmed to room temperature and stirred for 5 h. Additional Hunig's base (1.11 g, 1.5 mL, 8.61 mmol) and tert-butyl carbazate (420 mg. 3.18 mmol) were added and the reaction mixture was stirred for 18 h. The reaction was diluted with water (100 mL) and extracted with ethyl acetate (100 mL). The organic layer was washed with 1M HCl (100 mL), saturated aqueous sodium hydrogen carbonate (100 mL), water (100 mL) and brine (100 mL), dried over magnesium sulfate, filtered and concentrated. Purification by silica gel chromatography using 0 to 50% ethyl acetate in heptane gave tert-butyl N-[(4-benzyloxy-6-chloro-2-methyl-pyridine-3-carbonyl)amino]carbamate (808 mg, 70%) as a light yellow solid. ESI-MS m/z calc. 391.13. found 392.15 (M+1)⁺. ¹H-NMR (400 MHz, CDCl₃) δ 7.66 (br s, 1H), 7.43-7.34 (m, 5H), 6.80 (s, 1H), 6.61 (br s, 1H), 5.15 (s, 2H), 2.61 (s, 3H), 1.50 (s, 9H).

### Step 2: 4-benzyloxy-6-chloro-2-methyl-pyridine-3-carbohydrazide

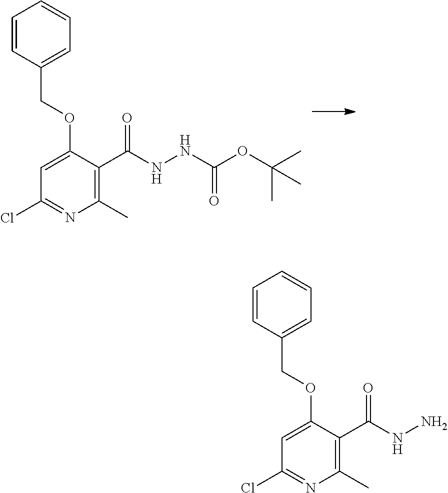 [SMILES: Cc1nc(Cl)cc(OCc2ccccc2)c1C(=O)NNC(=O)OC(C)(C)C ; Cc1nc(Cl)cc(OCc2ccccc2)c1C(=O)NN]

A solution of tert-butyl N-[(4-benzyloxy-6-chloro-2-methyl-pyridine-3-carbonyl)amino]carbamate (808 mg, 1.98 mmol) in HCl in dioxane (10 mL of 4 M, 40 mmol) was stirred at room temperature for 4 days. Diluted with diethyl ether, the solid was filtered off and dried to give 4-benzyloxy-6-chloro-2-methyl-pyridine-3-carbohydrazide (dihydrochloride salt) (722 mg, 95%) as a pale yellow solid. ESI-MS m/z calc. 291.07, found 292.04 (M+1)⁺. ¹H-NMR (400 MHz, DMSO-d₆) δ 11.72 (s, 1H), 7.44-7.33 (m, 5H), 7.27 (s, 1H), 5.29 (s, 2H), 2.37 (s, 3H).

### Step 3: 2-(4-benzyloxy-6-chloro-2-methyl-3-pyridyl)-5-methyl-1,3,4-oxadiazole (Intermediate A-27)

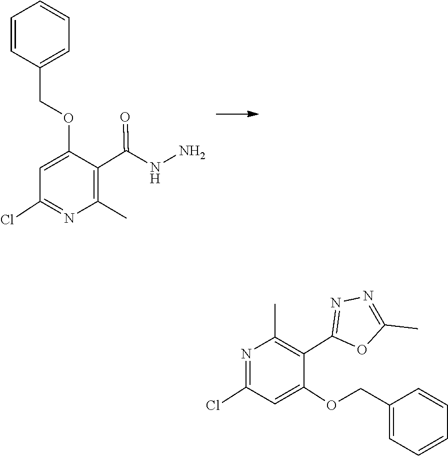 [SMILES: Cc1nc(Cl)cc(OCc2ccccc2)c1C(=O)NN ; Cc1nnc(-c2c(OCc3ccccc3)cc(Cl)nc2C)o1]

A solution of 4-benzyloxy-6-chloro-2-methyl-pyridine-3-carbohydrazide (100 mg, 0.27 mmol) in triethyl orthoacetate (885 mg, 1 mL, 5.45 mmol) was subjected to microwave irradiation at 120° C. for 1 h. The reaction mixture was cooled and partitioned between ethyl acetate (10 mL) and saturated aqueous sodium hydrogen carbonate (10 mL). The organic layer was separated and washed with brine (10 mL), dried over magnesium sulfate, filtered and concentrated. Purification by reverse phase column chromatography (C₁₈) using 5 to 95% acetonitrile in water containing 0.1% formic acid gave 2-(4-benzyloxy-6-chloro-2-methyl-3-pyridyl)-5-methyl-1,3,4-oxadiazole (Intermediate A-27, 45 mg, 44%). ESI-MS m/z calc. 315.07, found 316.04 (M+1)⁺. ¹H-NMR (400 MHz, CDCl₃) δ 7.39-7.30 (m, 5H), 6.86 (s, 1H), 5.15 (s, 2H), 2.58 (s, 3H), 2.52 (s, 3H).

### Intermediate A-28

### 4-(4-benzyloxy-6-chloro-2-methyl-3-pyridyl)oxazolidin-2-one

### Step 1: 4-benzyloxy-6-chloro-2-methyl-3-vinyl-pyridine

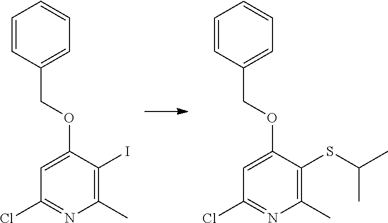 [SMILES: Cc1nc(Cl)cc(OCc2ccccc2)c1I ; Cc1nc(Cl)cc(OCc2ccccc2)c1SC(C)C]

A solution of 4-benzyloxy-6-chloro-3-iodo-2-methyl-pyridine (7.13 g, 19.44 mmol), vinyl boronic acid pinacol ester (3.63 g, 4 mL, 23.58 mmol), Pd(dppf)Cl₂ (1.43 g, 1.95 mmol) and sodium carbonate aqueous solution (30 mL of 2 M, 60 mmol) in dioxane (60 mL) was stirred at 70° C. under argon for 20 h. Cooled and partitioned between ethyl acetate (200 mL) and water (200 mL), the aqueous layer was further extracted with ethyl acetate (100 mL). The combined organic layer was washed with brine (200 mL), dried over magnesium sulfate, filtered and concentrated. Purification by silica gel column chromatography using 0 to 5% ethyl acetate in heptane gave 4-benzyloxy-6-chloro-2-methyl-3-vinyl-pyridine (4.79 g, 75%) as a yellow oil. ESI-MS m/z calc. 259.07, found 260.03 (M+1)⁺. ¹H-NMR (400 MHz, CDCl₃) δ 7.48-7.35 (m, 5H), 6.78 (s, 1H), 6.72-6.62 (m, 1H), 5.75 (dd, J=17.9, 1.8 Hz, 1H), 5.58 (dd, J=11.4, 1.8 Hz, 1H), 5.13 (s, 2H), 2.55 (s, 3H).

### Step 2: 4-(4-benzyloxy-6-chloro-2-methyl-3-pyridyl)oxazolidin-2-one (Intermediate A-28)

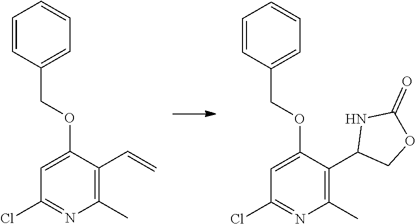 [SMILES: C=Cc1c(OCc2ccccc2)cc(Cl)nc1C ; Cc1nc(Cl)cc(OCc2ccccc2)c1C1COC(=O)N1]

To a solution of diphenyl diselenide (120 mg, 0.38 mmol), ammonium persulfate (105 mg, 0.46 mmol) and triflic acid (57.664 mg, 34 μL, 0.3842 mmol) in dioxane (1 mL) was added a solution of 4-benzyloxy-6-chloro-2-methyl-3-vinyl-pyridine (100 mg, 0.38 mmol) and ethyl carbamate (102 mg, 1.14 mmol) in dioxane (1 mL). The reaction was warmed to 100° C. and stirred for 18 h. Additional diphenyl diselenide (120 mg, 0.38 mmol), ammonium persulfate (105 mg, 0.46 mmol) and triflic acid (58 mg, 34 L, 0.38 mmol) were combined in dioxane (1 mL) and added to the reaction mixture, which was stirred for a further 18 h at 100° C. Additional ammonium persulfate (105 mg, 0.46 mmol) was added and the reaction stirred for a further 3 h. Cooled to room temperature and partitioned between ethyl acetate (20 mL) and water (20 mL), the organic layer was separated, washed with brine (20 mL), dried over magnesium sulfate, filtered and concentrated. Purification by reverse phase column chromatography (C₁₈) using 5 to 80% acetonitrile in water containing 0.1% formic acid gave 4-(4-benzyloxy-6-chloro-2-methyl-3-pyridyl)oxazolidin-2-one (Intermediate A-28, 20 mg, 14%). ESI-MS m/z calc. 318.07, found 319.07 (M+1)⁺. ¹H-NMR (400 MHz, CDCl₃) δ 7.44-7.36 (m, 5H), 6.84 (s, 1H), 5.37 (dd, J=10.1, 6.0 Hz, 1H), 5.18 (s, 2H).

### Intermediate A-29

### 5-(4-benzyloxy-6-chloro-2-methyl-3-pyridyl)-3-methyl-1,2,4-oxadiazole

### Step 1: 4-benzyloxy-6-chloro-2-methyl-pyridine-3-carbonyl chloride

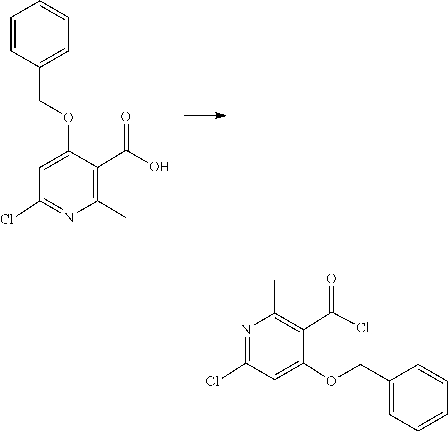 [SMILES: Cc1nc(Cl)cc(OCc2ccccc2)c1C(=O)O ; Cc1nc(Cl)cc(OCc2ccccc2)c1C(=O)Cl]

To a solution of 4-benzyloxy-6-choro-2-methyl-pyridine-3-carboxylic acid (200 mg, 0.67 mmol) in DCM (2 mL) at 0° C. was added oxalyl chloride (102 mg, 0.07 mL, 0.8 mmol) and DMF (11 mg, 12 μL, 0.15 mmol) and the mixture was warmed to room temperature for 4 h. The reaction mixture was concentrated under reduced pressure and co-evaporated with DCM (3×10 mL) to give crude 4-benzyloxy-6-chloro-2-methyl-pyridine-3-carbonyl chloride (200 mg, 100%) as a yellow oil.

### Step 2: 5-(4-benzyloxy-6-chloro-2-methyl-3-pyridyl)-3-methyl-1,2,4-oxadiazole (Intermediate A-29)

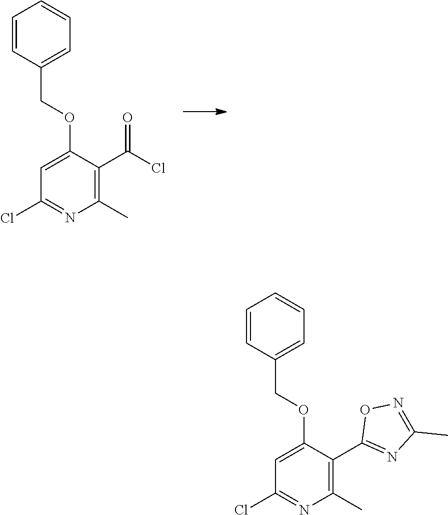 [SMILES: Cc1nc(Cl)cc(OCc2ccccc2)c1C(=O)Cl ; Cc1noc(-c2c(OCc3ccccc3)cc(Cl)nc2C)n1]

A mixture of 4-benzyloxy-6-chloro-2-methyl-pyridine-3-carbonyl chloride (2.22 g, 6.37 mmol), potassium carbonate (2.65 g, 19.17 mmol) and acetamide oxime (710 mg, 9.58 mmol) in DMF (20 mL) was stirred at room temperature for 3 days. Additional acetamide oxime (350 mg, 4.7245 mmol) and potassium carbonate (1.3 g, 9.4062 mmol) were added, the reaction was heated to 100° C. and stirring for 3 h. The reaction mixture was cooled to room temperature and partitioned between ethyl acetate (100 mL) and water (100 mL). The layers were separated and the organic layer was, washed with water (100 mL), brine (100 mL), dried over magnesium sulfate, filtered and concentrated. Purification by reverse phase column chromatography (C₁₈) using 5 to 95% acetonitrile in water, (0.1% formic acid) gave 5-(4-benzyloxy-6-chloro-2-methyl-3-pyridyl)-3-methyl-1,2,4-oxadiazole (Intermediate A-29, 383 mg, 18%) as an off-white solid. ESI-MS m/z calc. 315.07, found 316.06 (M+1)⁺. ¹H-NMR (400 MHz, CDCl₃) δ 7.41-7.32 (m, 5H), 6.87 (s, 1H), 5.19 (s, 2H), 2.52 (s, 3H), 2.51 (s, 3H).

### Intermediate A-30

### N-benzyl-N-(4-benzyloxy-6-chloro-2-methyl-3-pyridyl)methanesulfonamide

### Step 1: N-(4-benzyloxy-6-chloro-2-methyl-3-pyridyl)-N-methylsulfonyl-methanesulfonamide

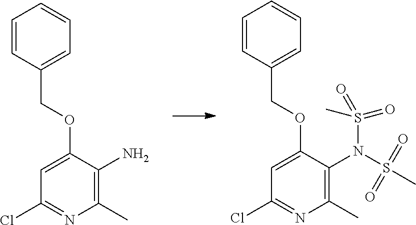 [SMILES: Cc1nc(Cl)cc(OCc2ccccc2)c1N ; Cc1nc(Cl)cc(OCc2ccccc2)c1N(S(C)(=O)=O)S(C)(=O)=O]

To 4-benzyloxy-6-chloro-2-methyl-pyridin-3-amine (514 mg, 2 mmol) in DCM (10 mL) at 0° C. was added DIPEA (890 mg, 1.2 mL, 6.9 mmol), followed by MsCl (266 mg, 180 μL, 2.33 mmol). The resulting mixture was stirred at this temperature for 2 h and additional MsCl (222 mg, 150 μL, 1.94 mmol) was added. After stirring for 1 h, the reaction mixture was quenched by addition of saturated aqueous ammonium chloride (20 mL) and water (40 mL). The reaction mixture was extracted with DCM (2×40 mL). The combined organic layer was washed with water (40 mL) and brine (40 mL), dried over sodium sulfate and concentrated in vacuo. The crude was purified by silica gel column chromatography using 20 to 80% EtOAc in heptanes to give N-(4-benzyloxy-6-chloro-2-methyl-3-pyridyl)-N-methylsulfonyl-methanesulfonamide (503 mg, 54%). ESI-MS m/z calc. 404.03, found 405.06 (M+1)⁺. ¹H-NMR (400 MHz, CDCl₃) δ 7.49-7.36 (m, 5H), 6.90 (s, 1H), 5.15 (s, 2H), 3.30 (s, 6H), 2.58 (s, 3H)

### Step 2: N-(4-benzyloxy-6-chloro-2-methyl-3-pyridyl)methanesulfonamide

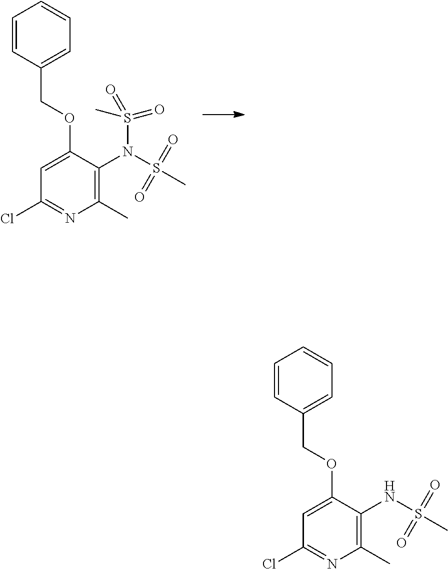 [SMILES: Cc1nc(Cl)cc(OCc2ccccc2)c1N(S(C)(=O)=O)S(C)(=O)=O ; Cc1nc(Cl)cc(OCc2ccccc2)c1NS(C)(=O)=O]

To N-(4-benzyloxy-6-chloro-2-methyl-3-pyridyl)-N-methylsulfonyl-methanesulfonamide (495 mg, 1.06 mmol) in dioxane (10 mL) was added NaOH (4.3 mL of 2 M, 8.6 mmol). The reaction mixture was stirred for 1 h at room temperature and acidified to pH 5 with 2N HCl. It was diluted with water (30 mL) then extracted with DCM (2×40 mL). The combined organic layer was washed with saturated brine, dried over sodium sulfate, filtered and concentrated to give N-(4-benzyloxy-6-chloro-2-methyl-3-pyridyl)methanesulfonamide (419 mg, 96%). ESI-MS m/z calc. 326.05, found 327.02 (M+1)⁺. ¹H-NMR (400 MHz, CDCl₃) δ 7.44-7.37 (m, 5H), 6.87 (s, 1H), 5.96 (s, 1H), 5.10 (s, 2H), 2.86 (s, 3H), 2.60 (s, 3H).

### Step 3: N-benzyl-N-(4-benzyloxy-6-chloro-2-methyl-3-pyridyl)methanesulfonamide (Intermediate A-30)

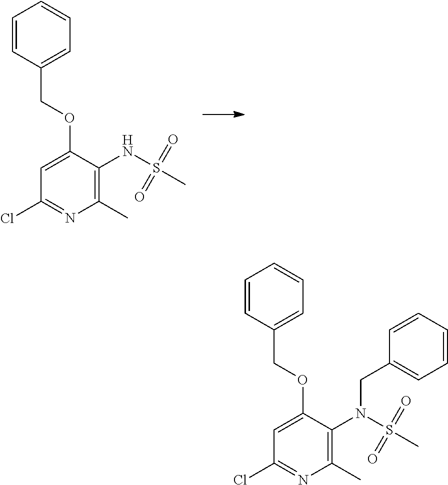 [SMILES: Cc1nc(Cl)cc(OCc2ccccc2)c1NS(C)(=O)=O ; Cc1nc(Cl)cc(OCc2ccccc2)c1N(Cc1ccccc1)S(C)(=O)=O]

To a solution of N-(4-benzyloxy-6-chloro-2-methyl-3-pyridyl)methanesulfonamide (419 mg, 1.03 mmol) in DMF (10 mL) at 0° C. was added NaH in oil (50 mg, 60% w/w, 1.25 mmol). The mixture stirred for 20 minutes at this temperature and benzyl bromide (259.20 mg, 180 μL, 1.5155 mmol) was added. Stirring was continued for another 1 h at 0° C. and then the reaction was quenched by the addition of saturated aqueous ammonium chloride (10 mL). The reaction mixture was diluted with water (30 mL) and extracted with EtOAc (2×35 mL). The combined organic layer was washed with water (30 mL) and brine (30 mL) then dried over sodium sulfate and concentrated in vacuo. The crude was purified by silica gel column chromatography using 20 to 80% EtOAc in heptane to obtain N-benzyl-N-(4-benzyloxy-6-chloro-2-methyl-3-pyridyl)methanesulfonamide (Intermediate A-30, 275 mg, 62%). ESI-MS m/z calc. 416.09, found 417.15 (M+1)⁺. ¹H-NMR (400 MHz, CDCl₃) δ 7.47-7.37 (m, 5H), 7.29-7.19 (m, 3H), 7.18-7.13 (m, 2H), 6.86 (s, 1H), 5.10 (s, 2H), 4.80 & 4.37 (ABq, J=13.7 Hz, 2H), 2.79 (s, 3H), 2.12 (s, 3H).

### Intermediate A-31

### (4-benzyloxy-2-chloro-6-methyl-3-pyridyl)methanol

### Step 1: (4-benzyloxy-2-chloro-6-methyl-3-pyridyl)methanol (Intermediate A-31)

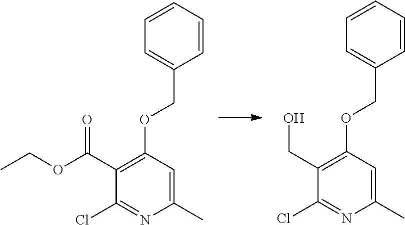 [SMILES: CCOC(=O)c1c(OCc2ccccc2)cc(C)nc1Cl ; Cc1cc(OCc2ccccc2)c(CO)c(Cl)n1]

ethyl 4-benzyloxy-2-chloro-6-methyl-pyridine-3-carboxylate (150 mg, 0.5 mmol) was dissolved in THF (1.5 mL) and was cooled to 0° C. LiAlH₄ (490 μL of 1 M, 0.49 mmol) in THF was added and the resulting solution was stirred for 4 hours at 0° C. and was gradually warmed up to room temperature. The resulting solution was diluted with EtOAc (20 mL), washed with a saturated solution of potassium sodium tartrate (Rochelle salt) and brine. The organic layer was dried over magnesium sulfate, filtered and concentrated under reduced pressure. The residue was purified by reverse phase column chromatography using 10 to 99% acetonitrile in water (5 mM HCl) yielding (4-benzyloxy-2-chloro-6-methyl-3-pyridyl)methanol (Intermediate A-31, 126.3 mg, 98%), as a white solid ESI-MS m/z calc. 263.07, found 164.122 (M+1)-. ¹H NMR (400 MHz, DMSO-d₆) δ 7.49 (d, J=7.0 Hz, 2H), 7.44-7.39 (m, 2H), 7.38-7.33 (m, 1H), 7.09 (s, 1H), 5.24 (s, 2H), 4.54 (s, 2H), 2.39 (s, 3H).

### Intermediate A-32

### 8-benzyloxy-6-chloro-3,4-dihydro-2H-thiopyrano[3,2-b]pyridine

### Step 1: ethyl 5-amino-3,4-dihydro-2H-thiopyran-6-carboxylate

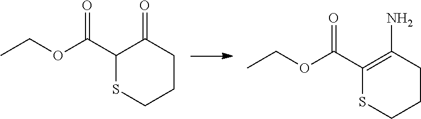

To ethyl 3-oxotetrahydrothiopyran-2-carboxylate (850 mg, 3.94 mmol) in ethanol (15 mL) was added ammonium acetate (1 g, 12.97 mmol) and the reaction mixture was refluxed for 2 h. The reaction was concentrated under reduced pressure. It was taken up in EtOAc (10 mL), washed with water (3×5 mL), dried over sodium sulfate, filtered and concentrated under reduced pressure to give ethyl 5-amino-3,4-dihydro-2H-thiopyran-6-carboxylate (702 mg, 82%). ESI-MS m/z calc. 187.06, found 188.06 (M+1)⁺. ¹H-NMR (400 MHz, CDCl₃) δ 6.32 (br s, 2H), 4.21 (q, J=7.1 Hz, 2H), 2.79 (t, J=6.3 Hz, 2H), 2.36 (t, J=6.5 Hz, 2H), 2.09-2.03 (m, 2H), 1.33 (t, J=7.2 Hz, 3H).

### Step 2: ethyl 5-[(3-ethoxy-3-oxo-propanoyl)amino]-3,4-dihydro-2H-thiopyran-6-carboxylate

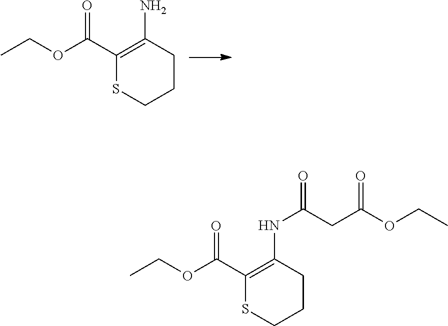 [SMILES: CCOC(=O)C1=C(N)CCCS1 ; CCOC(=O)CC(=O)NC1=C(C(=O)OCC)SCCC1]

To ethyl 5-amino-3,4-dihydro-2H-thiopyran-6-carboxylate (127 mg, 0.58 mmol) in dichloromethane (2 mL) at 0° C. was added potassium bicarbonate (300 mg, 3 mmol). The reaction mixture was stirred for 5 minutes and ethyl 3-chloro-3-oxo-propanoate (130 mg, 0.86 mmol) in dichloromethane (500 L) was added dropwise. The reaction mixture was stirred at room temperature for 2 h. The solids were filtered off and the filtrate was concentrated under reduced pressure to give ethyl 5-[(3-ethoxy-3-oxo-propanoyl)amino]-3,4-dihydro-2H-thiopyran-6-carboxylate (190 mg, 87%) as a yellow gum. ¹H-NMR (400 MHz, CDCl₃) δ 11.66 (br s, 1H), 4.30-4.22 (m, 4H), 3.40 (s, 2H), 3.09 (t, 2H, J=6.4 Hz), 2.83-2.80 (m, 2H), 2.10-2.04 (m, 2H), 1.37-1.30 (m, 6H).

### Step 3: ethyl 8-hydroxy-6-oxo-2,3,4,5-tetrahydrothiopyrano[3,2-b]pyridine-7-carboxylate

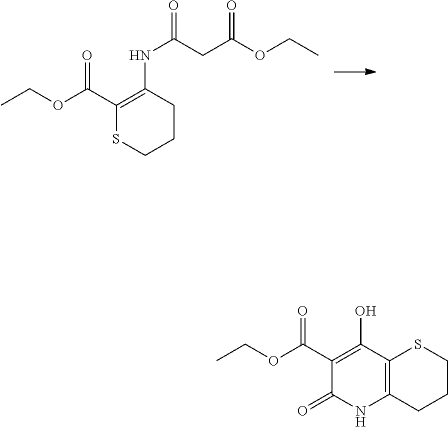

To ethyl 5-[(3-ethoxy-3-oxo-propanoyl)amino]-3,4-dihydro-2H-thiopyran-6-carboxylate (12.11 g, 37.95 mmol) in ethanol (120 mL) was added sodium ethoxide (13.6 g, 199.85 mmol) portionwise. The reaction was stirred at 70° C. for 1 h then cooled to 0° C. and brought to pH 4 using 2 molar aqueous HCl. The reaction mixture was filtered, the precipitate was washed with cold water and dried under reduced pressure to give ethyl 8-hydroxy-6-oxo-2,3,4,5-tetrahydrothiopyrano[3,2-b]pyridine-7-carboxylate (10.2 g, 100%). ESI-MS m/z calc. 255.05, found 256.01 (M+1)⁺. ¹H-NMR (400 MHz, DMSO-d₆) δ 13.67 (s, 1H), 11.46 (s, 1H), 4.31 (q, J=7.1 Hz, 2H), 2.92-2.89 (m, 2H), 2.60 (t, J=6.3 Hz, 2H), 2.04-1.98 (m, 2H), 1.29 (t, J=7.1 Hz, 3H).

### Step 4: 8-hydroxy-2,3,4,5-tetrahydrothiopyrano[3,2-b]pyridin-6-one

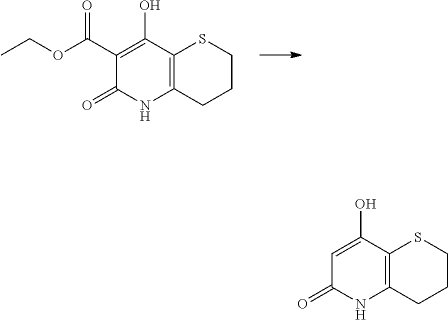 [SMILES: CCOC(=O)c1c(O)c2c([nH]c1=O)CCCS2 ; O=c1cc(O)c2c([nH]1)CCCS2]

ethyl 8-hydroxy-6-oxo-2,3,4,5-tetrahydrothiopyrano[3,2-b]pyridine-7-carboxylate (160 mg, 0.58 mmol) in aqueous sodium hydroxide (3.1 mL of 2 M, 6.2 mmol) was refluxed for 3 hours. The reaction was cooled to 5° C. and adjusted to pH 7 using aqueous citric acid. The reaction mixture was filtered and the precipitate was washed with cold water concentrated under reduced pressure to give crude 8-hydroxy-2,3,4,5-tetrahydrothiopyrano[3,2-b]pyridin-6-one (106.97 mg, 100%) as an off white solid. ESI-MS m/z calc. 183.03, found 184.0 (M+1)⁺.

### Step 5: 6,8-dichloro-3,4-dihydro-2H-thiopyrano[3,2-b]pyridine

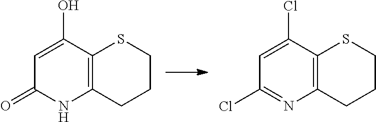 [SMILES: O=c1cc(O)c2c([nH]1)CCCS2 ; Clc1cc(Cl)c2c(n1)CCCS2]

To phosphorus oxychloride (197.40 g, 120 mL, 1.29 mol) was added 8-hydroxy-2,3,4,5-tetrahydrothiopyrano[3,2-b]pyridin-6-one (6.9445 g, 37.901 mmol) portionwise. The reaction was stirred at reflux for 16 h. The reaction was cooled to room temperature and concentrated under reduce pressure and azeotroped with toluene (2×15 mL). The crude mixture was added portionwise to iced water (100 mL) then extracted with EtOAc (3×100 mL). Purification by silica gel chromatography using 0 to 100% EtOAc in heptane yielded 6,8-dichloro-3,4-dihydro-2H-thiopyrano[3,2-b]pyridine (4.25 g, 42%). ¹H-NMR (400 MHz, CDCl₃) δ 7.20 (s, 1H), 3.10-3.07 (m, 2H), 3.03-2.99 (m, 2H), 2.28-2.19 (m, 2H).

### Step 6: 8-benzyloxy-6-chloro-3,4-dihydro-2H-thiopyrano[3,2-b]pyridine (Intermediate A-32)

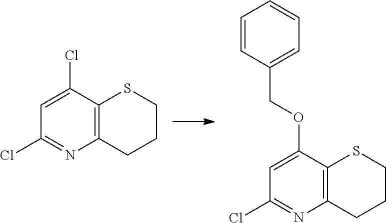 [SMILES: Clc1cc(Cl)c2c(n1)CCCS2 ; Clc1cc(OCc2ccccc2)c2c(n1)CCCS2]

To dry DMF (20 mL) under argon gas was added benzyl alcohol (490 mg, 4.53 mmol). The solution was cooled to 0° C. and sodium hydride (60% on mineral oil) (183 mg, 4.57 mmol) was added. The mixture was stirred for 1 h then 6,8-dichloro-3,4-dihydro-2H-thiopyrano[3,2-b]pyridine (950 mg, 4.25 mmol) was added. The reaction mixture was stirred at 0° C. for 4 h. The reaction was quenched with saturated aqueous ammonium chloride (20 mL), extracted with EtOAc (40 mL), washed with water (3×30 mL), brine (30 mL), dried over sodium sulfate, filtered and concentrated under reduced pressure. Purification by silica gel chromatography using 0 to 20% EtOAc in heptane yielded the product 8-benzyloxy-6-chloro-3,4-dihydro-2H-thiopyrano[3,2-b]pyridine (Intermediate A-32, 900 mg, 72%) as an off white solid. ESI-MS m/z calc. 291.05, found 292.03 (M+1)⁺. ¹H-NMR (400 MHz, CDCl₃) δ 7.46-7.43 (m, 4H), 7.41-7.35 (m, 1H), 6.69 (s, 1H), 5.19 (s, 2H), 3.03-3.00 (m, 2H), 2.97 (t, J=6.3 Hz, 2H), 2.26-2.20 (m, 2H). ESI-MS m/z calc. 291.05, found 292.03 (M+1)⁺.

### Intermediate A-33

### 8-benzyloxy-6-chloro-2-methyl-3,4-dihydropyrido[2,3-e]thiazine 1,1-dioxide

### Step 1: 2,4-dimethoxy-5-[(4-methoxyphenyl)methylsulfanyl]pyridine

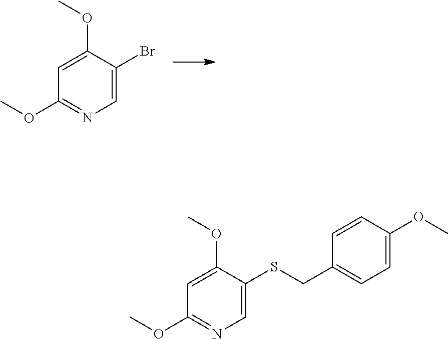

A mixture of 5-bromo-2,4-dimethoxy-pyridine (6.04 g, 27.67 mmol), Xantphos (640 mg, 1.11 mmol), DIPEA (11.13 g, 15 mL, 86.12 mmol) and (4-methoxyphenyl)methanethiol (5.56 g, 5 mL, 36.05 mmol) in dioxane (120 mL) in a flamed-dried flask was bubbled with nitrogen for 10 min. Pd₂(dba)₃ (508 mg, 0.55 mmol) was added and the reaction mixture was stirred at 100° C. for 17 h with a Vigreux column. The reaction mixture was cooled to room temperature and diluted with ethyl acetate (250 mL). The organic layer was washed with water (3×75 mL) and brine (75 mL), dried over anhydrous sodium sulfate, filtered and concentrated under reduced pressure. Purification by silica gel column chromatography using 0 to 40% EtOAc in heptanes gave 2,4-dimethoxy-5-[(4-methoxyphenyl)methylsulfanyl]pyridine (8.34 g, 98%) as an orange oil. ESI-MS m/z calc. 291.09, found 292.2 (M+1)⁺. ¹H NMR (400 MHz, CDCl₃) δ 7.89 (s, 1H), 7.09 (d, J=8.7 Hz, 2H), 6.78 (d, J=8.7 Hz, 2H), 6.18 (s, 1H), 3.90 (s, 3H), 3.89 (s, 2H), 3.87 (s, 3H), 3.78 (s, 3H).

### Step 2: 4,6-dimethoxypyridine-3-sulfonyl chloride

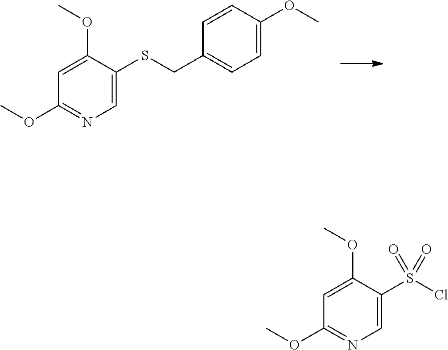

NCS (7 g, 52.42 mmol) was added to a solution of 2,4-dimethoxy-5-[(4-methoxyphenyl)methylsulfanyl]pyridine (3.95 g, 12.55 mmol) in acetic acid (70 mL) and water (17 mL). The reaction mixture was stirred at room temperature for 1 h and diluted with ethyl acetate (400 mL). The organic layer was washed with water (2×100 mL), water/brine 1/1 (200 mL), saturated aqueous KHCO₃ (3×100 mL), saturated aqueous KHCO₃/10% aqueous Na₂S₂O₃ 1/1 (100 mL) and brine (100 mL), dried over anhydrous sodium sulfate, filtered and concentrated. Purification by silica gel column chromatography with 0 to 50% ethyl acetate in heptanes, followed by trituration with heptane/MTBE 9/1 (10 mL) gave 4,6-dimethoxypyridine-3-sulfonyl chloride (2.51 g, 84%) as a white solid. ESI-MS m/z calc. 236.98, found 238.1 (M+1)⁺. ¹H NMR (400 MHz, CDCl₃) δ 8.63 (s, 1H), 6.32 (s, 1H), 4.04 (s, 3H), 4.02 (s, 3H).

### Step 3: 4,6-dimethoxy-N-methyl-pyridine-3-sulfonamide

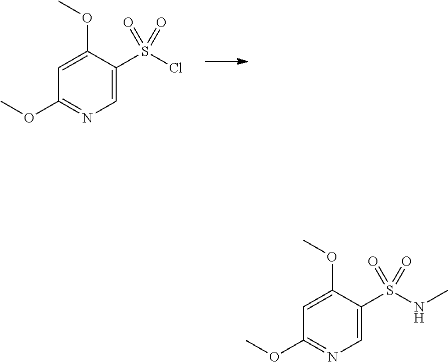

To a solution of 4,6-dimethoxypyridine-3-sulfonyl chloride (1 g, 4.19 mmol) in dichloromethane (25 mL) was added methylamine (in tetrahydrofuran) (6.5 mL of 2 M, 13 mmol) and the reaction mixture was stirred at room temperature for 1.5 h. It was diluted with dichloromethane (100 mL). The organic layer was washed with water (2×30 mL) and brine (30 mL), dried over anhydrous sodium sulfate, filtered and concentrated to obtain 4,6-dimethoxy-N-methyl-pyridine-3-sulfonamide (920 mg, 94%) as an off-white solid. ESI-MS m/z calc. 232.05, found 233.1 (M+1)⁺. ¹H NMR (400 MHz, CDCl₃) δ 8.55 (s, 1H), 6.28 (s, 1H), 4.63 (q, J=5.4 Hz, 1H), 3.99 (s, 3H), 3.97 (s, 3H), 2.62 (d, J=5.4 Hz, 3H).

### Step 4: N-(2-bromoethyl)-4,6-dimethoxy-N-methyl-pyridine-3-sulfonamide

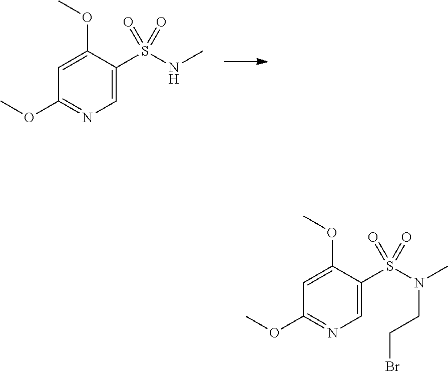

A mixture of 4,6-dimethoxy-N-methyl-pyridine-3-sulfonamide (1 g, 4.31 mmol), 1,2-dibromoethane (12.21 g, 5.6 mL, 64.98 mmol) and potassium carbonate (1.8 g, 13.024 mmol) in acetonitrile (40 mL) was stirred at 120° C. for 27 h. The mixture was cooled to room temperature and diluted with ethyl acetate (100 mL). The organic layer was washed with water (20 mL) and brine (20 mL), dried over anhydrous sodium sulfate, filtered and concentrated. Purification by silica gel column chromatography using 0 to 40% ethyl acetate in heptanes gave N-(2-bromoethyl)-4,6-dimethoxy-N-methyl-pyridine-3-sulfonamide (1.12 g, 72%). ESI-MS m/z calc. 337.99, found 339.0 (M+1)⁺. ¹H NMR (400 MHz, CDCl₃) δ 8.55 (s, 1H), 6.24 (s, 1H), 3.97 (s, 3H), 3.93 (s, 3H), 3.60-3.52 (m, 2H), 3.51-3.44 (m, 2H), 2.90 (s, 3H).

### Step 5: O-ethyl 2-[(4,6-dimethoxy-3-pyridyl)sulfonyl-methyl-amino]ethylsulfanylmethanethioate

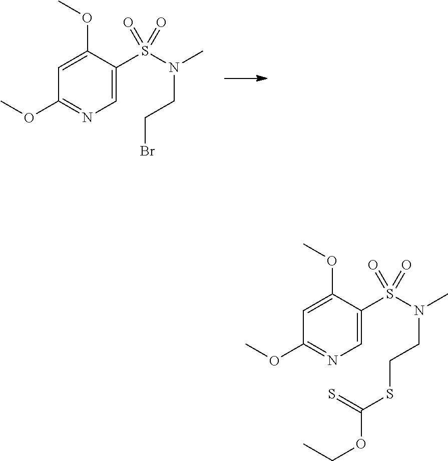

A mixture of N-(2-bromoethyl)-4,6-dimethoxy-N-methyl-pyridine-3-sulfonamide (4.67 g, 13.57 mmol) and ethoxycarbothioylsulfanylpotassium (3.26 g, 20.34 mmol) in acetone was stirred at room temperature for 21 h. The solvent was evaporated under reduced pressure and the resulting residue was diluted with ethyl acetate (200 mL). The organic layer was washed with water (100 mL) and brine (100 mL), dried over anhydrous sodium sulfate, filtered and concentrated. Purification by silica gel column chromatography using 0 to 40% ethyl acetate in heptanes gave O-ethyl 2-[(4,6-dimethoxy-3-pyridyl)sulfonyl-methyl-amino]ethylsulfanylmethanethioate (4.3 g, 82%). ESI-MS m/z calc. 380.05, found 381.1 (M+1)⁺. ¹H NMR (400 MHz, CDCl₃) δ 8.56 (s, 1H), 6.24 (s, 1H), 4.64 (q, J=7.1 Hz, 2H), 3.99 (s, 3H), 3.93 (s, 3H), 3.50-3.42 (m, 2H), 3.35-3.28 (m, 2H), 2.95 (s, 3H), 1.43 (t, J=7.1 Hz, 3H).

### Step 6: 6,8-dimethoxy-2-methyl-3,4-dihydropyrido[2,3-e]thiazine 1,1-dioxide

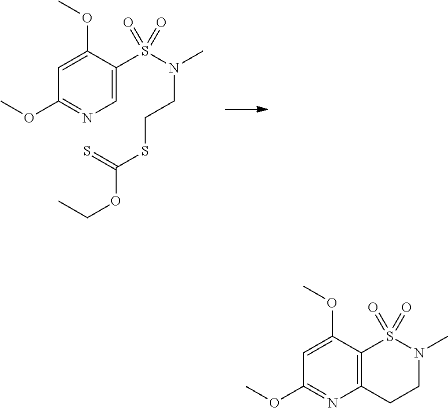

A solution of O-ethyl 2-[(4,6-dimethoxy-3-pyridyl)sulfonyl-methyl-amino]ethylsulfanylmethanethioate (1 g, 2.5 mmol) and TFA (740 mg, 0.5 mL, 6.5 mmol) in ethyl acetate (40 mL) was refluxed for 15 minutes. Lauroyl peroxide (1.96 g mg, 4.9 mmol) was added portionwise over 4 hours. One hour after the last addition, the reaction mixture was cooled to room temperature and concentrated. The residue was diluted with acetonitrile (75 mL) and washed with heptanes (3×50 mL). The solvent was evaporated and the crude was purified via silica gel column chromatography using 0 to 50% EtOAvc in heptanes to obtain 6,8-dimethoxy-2-methyl-3,4-dihydropyrido[2,3-e]thiazine 1,1-dioxide (305 mg, 46%). ESI-MS m/z calc. 258.06, found 259.1 (M+1)⁺. ¹H NMR (400 MHz, CDCl₃) δ 6.16 (s, 1H), 3.96 (s, 3H), 3.93 (s, 3H), 3.83 (t, J=6.2 Hz, 2H), 2.99-2.94 (m, 5H).

### Step 7: 2-methyl-1,1-dioxo-3,4-dihydropyrido[2,3-e]thiazine-6,8-diol

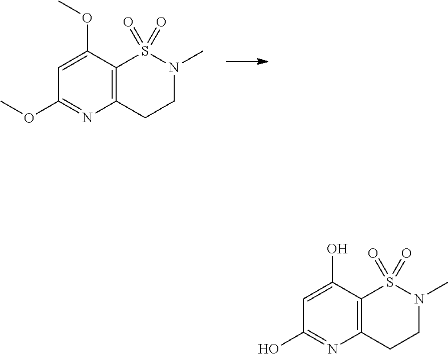

A mixture of 6,8-dimethoxy-2-methyl-3,4-dihydropyrido[2,3-e]thiazine 1,1-dioxide (20 mg, 0.07 mmol) and HBr (in water) (745 mg, 0.5 mL of 48% w/v, 2.96 mmol) was stirred at 100° C. for 23 h. It was cooled to room temperature and solid sodium hydroxide (170 mg) was added portionwise. A few drops of 2 N aqueous NaOH were added until pH 3-4 was reached. The solvent was evaporated under reduced pressure. The residue was coevaporated with toluene (2×3 mL) and dried under high vacuum to give 2-methyl-1,1-dioxo-3,4-dihydropyrido[2,3-e]thiazine-6,8-diol (615 mg, 100%). ESI-MS m/z calc. 230.03, found 231.1 (M+1)⁺.

### Step 8: 6,8-dichloro-2-methyl-3,4-dihydropyrido[2,3-e]thiazine-1,1-dioxide

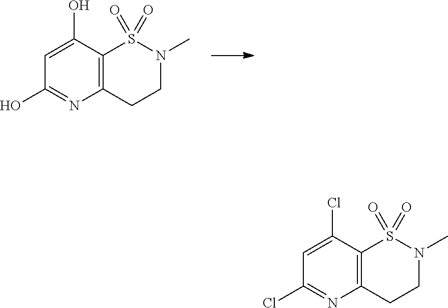

To stirred POCl₃ (82.25 g, 50 mL, 536.42 mmol) at 40° C. was added portionwise crude 2-methyl-1,1-dioxo-3,4-dihydropyrido[2,3-e]thiazine-6,8-diol (10.9 g, 1.85 mmol). The reaction mixture was stirred at 70° C. for 3.5 h and at 100° C. for 2.5 h. It was cooled to room temperature and excess POCl₃ was removed under reduced pressure. The residue was co-evaporated with toluene (25 mL) and was poured onto a stirring mixture of ethyl acetate (100 mL), water (200 mL) and sodium carbonate (20 g). The layers were separated. The aqueous layer was extracted with ethyl acetate (2×200 mL). The organic layers were combined and washed with brine (50 mL), dried over anhydrous sodium sulfate, filtered and evaporated under reduced pressure. The crude product was purified by silica gel chromatography using 0-50% ethyl acetate in heptanes to give 6,8-dichloro-2-methyl-3,4-dihydropyrido[2,3-e]thiazine-1,1-dioxide (288 mg, 49%) as a white solid. ESI-MS m/z calc. 265.97, found 267.0 (M+1)⁺.

### Step 9: 8-benzyloxy-6-chloro-2-methyl-3,4-dihydropyrido[2,3-e]thiazine-1,1-dioxide (Intermediate A-33)

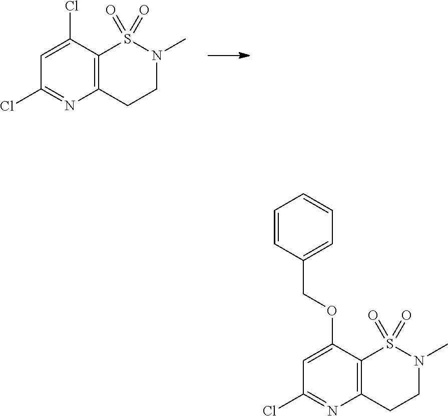 [SMILES: CN1CCc2nc(Cl)cc(Cl)c2S1(=O)=O ; CN1CCc2nc(Cl)cc(OCc3ccccc3)c2S1(=O)=O]

To a solution of 6,8-dichloro-2-methyl-3,4-dihydropyrido[2,3-e]thiazine-1,1-dioxide (288 mg, 0.90 mmol) and phenylmethanol (1.25 g, 1.2 mL, 11. 6 mmol) in THF (6 mL) and water (1.5 mL) was added lithium hydroxide monohydrate (47 mg, 1.12 mmol) and the reaction mixture was stirred at room temperature for 72 hours. The reaction mixture was diluted with sodium bicarbonate saturated aqueous solution (10 mL) and water (10 mL). This aqueous layer was extracted with MTBE (3×15 mL). The combined organic layers were dried over anhydrous sodium sulfate, filtered and concentrated under reduced pressure. The residue was purified by reverse phase chromatography using 5 to 100% of acetonitrile in water containing 0.1% formic acid to afford 8-benzyloxy-6-chloro-2-methyl-3,4-dihydropyrido[2,3-e]thiazine 1,1-dioxide (Intermediate A-33, 46 mg, 13%) as a white solid. ¹H NMR (400 MHz, CDCl₃) δ 7.54-7.49 (m, 2H), 7.46-7.40 (m, 2H), 7.40-7.34 (m, 1H), 6.87 (s, 1H), 5.34 (s, 2H), 3.89 (t, J=6.1 Hz, 2H), 3.09 (t, J=6.1 Hz, 2H), 2.99 (s, 3H). ¹³C NMR (101 MHz, CDCl₃) δ 164.3, 156.5, 154.4, 134.1, 128.9, 128.6, 126.8, 122.6, 107.7, 71.4, 46.0, 35.2, 26.3. ESI-MS m/z calc. 338.05, found 339.1 (M+1)⁺.

### Example 2—Preparation of Intermediates B-1 to B-17

### Intermediate B-1

### 4,4,5,5-tetramethyl-2-[2-methyl-4-[1-(trifluoromethyl)cyclopropyl]phenyl]-1,3,2-dioxaborolane

### Step 1: 1-bromo-2-methyl-4-[1-(trifluoromethyl)cyclopropyl]benzene

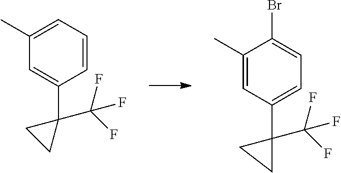

Under an inert atmosphere, acetic acid (10 mL) was placed in a 50 mL flask and 1-methyl-3-[1-(trifluoromethyl)cyclopropyl]benzene (1 g, 5 mmol) was added. Then, bromine (310 μL, 6 mmol) was added and the mixture was stirred for 18 h at 15˜20° C. The mixture was added to ice and stirred for 20 min. Ethyl acetate was added, and the layers were separated. The organic layer was washed with sodium bicarbonate (3×), followed by water (2×). The organic layer was dried over magnesium sulfate, filtered, and concentrated. The crude material was purified via silica gel column chromatography using 0 to 5% ethyl acetate in hexanes to obtain 1-bromo-2-methyl-4-[1-(trifluoromethyl)cyclopropyl]benzene (1.12 g, 80%). ¹H NMR (400 MHz, CDCl₃) δ 7.49 (d, J=8.2 Hz, 1H), 7.32 (d, J=2.2 Hz, 1H), 7.13 (dd, J=8.3, 2.3 Hz, 1H), 2.39 (s, 3H), 1.36-1.31 (m, 2H), 1.03-0.96 (m, 2H) ppm.

### Step 2: 4,4,5,5-tetramethyl-2-[2-methyl-4-[1-(trifluoromethyl)cyclopropyl]phenyl]-1,3,2-dioxaborolane (Intermediate B-1)

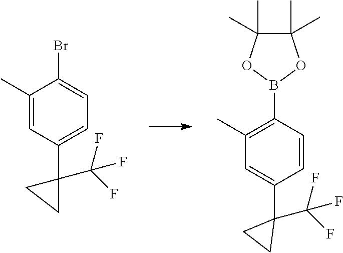 [SMILES: Cc1cc(C2(C(F)(F)F)CC2)ccc1Br ; Cc1cc(C2(C(F)(F)F)CC2)ccc1B1OC(C)(C)C(C)(C)O1]

A microwave vial charged with 1-bromo-2-methyl-4-[1-(trifluoromethyl)cyclopropyl]benzene (3.1 g, 11.11 mmol), bis(pinacolato)diboron (8.7 g, 34.26 mmol), Potassium acetate (2.3 g, 23.20 mmol), Pd(dppf)₂Cl₂-DCM (910 mg, 1.11 mmol) and 1,4-dioxane (30 mL) was degassed under nitrogen, scaled and heated at 90° C. for 16 h. The mixture was filtered through a plug of Celite®, the solvent was evaporated and the crude material was purified via silica gel column chromatography using 0 to 10% ethyl acetate in hexanes to obtain 4,4,5,5-tetramethyl-2-[2-methyl-4-[1-(trifluoromethyl)cyclopropyl]phenyl]-1,3,2-dioxaborolane (Intermediate B-1, 2.86 g, 79%) as a pale yellow solid. ¹H NMR (400 MHz, CDCl₃) δ 7.74 (d, J=8.1 Hz, 1H), 7.26-7.24 (m, 2H), 2.54 (s, 3H), 1.36-1.30 (m, 14H), 1.04-0.98 (m, 2H) ppm.

### Intermediate B-2

### 2-(4-tert-butyl-5-chloro-2-methyl-phenyl)-4,4,5,5-tetramethyl-1,3,2-dioxaborolane

### Step 1: 1-(4-bromo-2-chloro-5-methyl-phenyl)ethanone

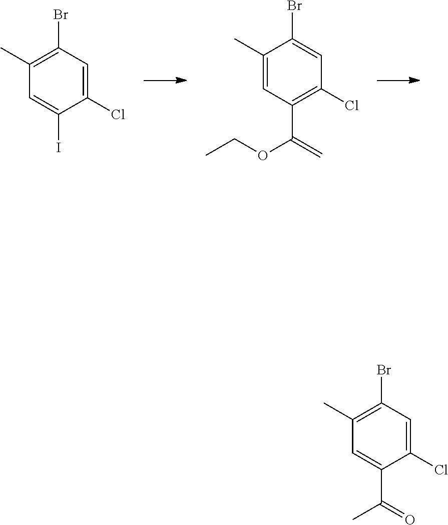

To a microwave vial containing a solution of 1-bromo-5-chloro-4-iodo-2-methyl-benzene (21 g, 63.37 mmol) and tributyl(1-ethoxyvinyl)stannane (21.4 mL, 63.34 mmol) in 1,4-dioxane (105 mL) was added PdCl₂(PPh₃)₂(2.25 g, 3.21 mmol). The mixture was degassed with nitrogen for 30-60 seconds, sealed and heated at 100° C. for 17 h. The mixture cooled to room temperature and quenched with water. The aqueous layer was extracted with DCM (3×). The combined organic layer was dried over magnesium sulfate, filtered and concentrated to obtain 1-bromo-5-chloro-4-(1-ethoxyvinyl)-2-methyl-benzene. ESI-MS m/z calc. 273.98, found 277.07 (M+2)+.

The above intermediate was taken up in THF (100 mL) and HCl (95 mL of 1 M, 95 mmol) was added, and the mixture was stirred at room temperature for 1 h. The aqueous layer was extracted with DCM (3×), dried over magnesium sulfate, filtered, and concentrated. The crude material was purified via silica gel column chromatography using 0 to 15% ethyl acetate in hexanes to obtain 1-(4-bromo-2-chloro-5-methyl-phenyl)ethanone (12.62 g, 80%) as a white solid. ESI-MS m/z calc. 247.94, found 248.95 (M+1)⁺. ¹H NMR (400 MHz, DMSO-d₆) δ 7.83 (s, 1H), 7.74 (s, 1H), 2.58 (s, 3H), 2.38 (s, 3H) ppm.

### Step 2: 1-bromo-4-tert-butyl-5-chloro-2-methyl-benzene

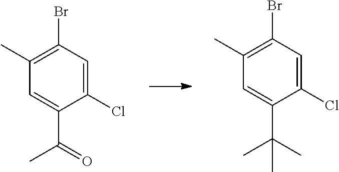

To a solution of tetrachlorotitanium in toluene (50 mL of 1 M, 50 mmol) in DCM (35 mL) at −40° C. was added dimethylzinc in toluene (33 mL of 2 M, 66 mmol) slowly maintaining the temperature below −40° C. (internal temperature). The mixture was stirred at −40° C. for 30 min and then a solution of 1-(4-bromo-2-chloro-5-methyl-phenyl)ethanone (6.3 g, 25.45 mmol) in DCM (10 mL) was added dropwise, maintaining the internal temperature below −40° C. The mixture was gradually warmed to room temperature and stirred for 2 h. The mixture was quenched by slowly pouring it into an ice and saturated sodium bicarbonate solution. The aqueous phase was acidified with concentrated HCl and then extracted with DCM. The organic phases were combined, dried over magnesium sulfate, filtered, and concentrated in vacuo. The crude material was purified via silica gel column chromatography using hexanes to obtain 1-bromo-4-tert-butyl-5-chloro-2-methyl-benzene (5.8 g, 87%). ¹H NMR (400 MHz, DMSO-d₆) δ 7.60 (s, 1H), 7.42 (s, 1H), 2.33 (s, 3H), 1.41 (s, 9H) ppm.

### Step 3: 2-(4-tert-butyl-5-chloro-2-methyl-phenyl)-4,4,5,5-tetramethyl-1,3,2-dioxaborolane (Intermediate B-2)

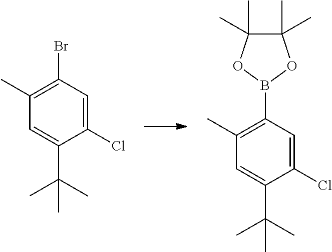 [SMILES: Cc1cc(C(C)(C)C)c(Cl)cc1Br ; Cc1cc(C(C)(C)C)c(Cl)cc1B1OC(C)(C)C(C)(C)O1]

2-(4-tert-butyl-5-choro-2-met y-phenyl)-4,4,5,5-tetramethyl-1,3,2-dioxaborolane (Intermediate B-2) was prepared from 1-bromo-4-tert-butyl-5-chloro-2-methyl-benzene using procedure analogous to that found in Intermediate B-1, Step 2 as a pale-yellow solid. ¹H NMR (400 MHz, CDCl₃) δ 7.71 (s, 1H), 7.20 (s, 1H), 2.49 (s, 3H), 1.46 (s, 9H), 1.33 (s, 12H) ppm.

### Intermediate B-3

### 2-[2,5-dimethyl-4-(2,2,2-trifluoro-1,1-dimethyl-ethyl)phenyl]-4,4,5,5-tetramethyl-1,3,2-dioxaborolane

### Step 1: 1-(4-bromo-2,5-dimethyl-phenyl)-2,2,2-trifluoro-ethanone

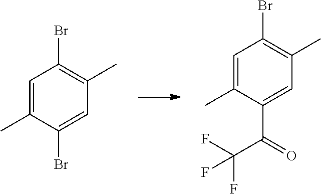

To a solution of 1,4-dibromo-2,5-dimethyl-benzene (9.5 g, 36 mmol) in tetrahydrofuran (180 mL) cooled to −78° C. under an atmosphere of nitrogen was added n-BuLi (2.5 M in hexanes) (16 mL of 2.5 M, 40 mmol) dropwise over 40 min. The mixture was stirred at −74° C. for 40 min. Ethyl 2,2,2-trifluoroacetate (5.73 g, 4.8 mL, 40.34 mmol) was added dropwise over 15 min and the mixture was stirred for an additional 30 min at −74° C. The mixture was carefully quenched by dropwise addition of a mixture of hydrochloric acid (8.9 mL of 37% w/v, 90 mmol) and ethanol (6 mL) precooled to −78° C. After stirring for 20 min, the mixture was warmed to room temperature. The mixture was diluted with water (100 mL). The organic layer was separated, dried over magnesium sulfate, filtered, and concentrated under reduced pressure. The crude product was purified using silica gel chromatography using 100% heptanes to afford 1-(4-bromo-2,5-dimethyl-phenyl)-2,2,2-trifluoro-ethanone (8.55 g, 79%) as a colorless oil. ¹H NMR (400 MHz, CDCl₃) δ 7.70 (s, 1H), 7.57 (s, 1H), 2.52 (s, 3H), 2.46 (s, 3H). ¹⁹F NMR (377 MHz, CDCl₃) δ −71.21 (s, 3F) ppm.

### Step 2: 2-(4-bromo-2,5-dimethyl-phenyl)-1,1,1-trifluoro-propan-2-ol

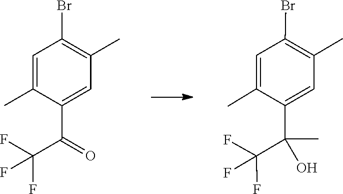

To a solution of 1-(4-bromo-2,5-dimethyl-phenyl)-2,2,2-trifluoro-ethanone (945 mg, 3.36 mmol) in tetrahydrofuran (15 mL) cooled to 0° C. was slowly added a solution of methylmagnesium bromide in diethyl ether (3.3 mL of 3 M, 9.9 mmol) and the mixture was stirred at 50° C. for 1 h. The mixture was cooled to 0° C. and quenched slowly with water, then with a saturated solution of ammonium chloride. The organic layer was separated, and the aqueous layer was extracted with ethyl acetate (3×25 mL). The combined organic layer was washed with brine, dried over anhydrous magnesium sulfate, filtered, and concentrated in vacuo to provide the crude product. Purification by silica gel chromatography using 0 to 10% ethyl acetate in heptanes provided 2-(4-bromo-2,5-dimethyl-phenyl)-1,1,1-trifluoro-propan-2-ol (812 mg, 78%). ESI-MS m/z calc. 296, found 279.0 (M-17)⁺. ¹H NMR (400 MHz, CDCl₃) δ 7.39 (s, 1H), 7.29 (s, 1H), 2.54 (s, 3H), 2.38 (s, 3H), 2.29 (s, 1H) 1.87-1.84 (m, 3H) ppm. ¹⁹F NMR (377 MHz, CDCl₃) δ −80.01 (s, 3F) ppm.

### Step 3: [1-(4-bromo-2,5-dimethyl-phenyl)-2,2,2-trifluoro-1-methyl-ethyl]methanesulfonate

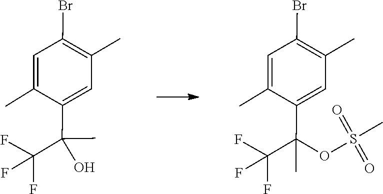 [SMILES: Cc1cc(C(C)(O)C(F)(F)F)c(C)cc1Br ; Cc1cc(C(C)(OS(C)(=O)=O)C(F)(F)F)c(C)cc1Br]

A solution of 2-(4-bromo-2,5-dimethyl-phenyl)-1,1,1-trifluoro-propan-2-ol (2 g, 6.32 mmol) in tetrahydrofuran (6 mL) was added dropwise at room temperature to a suspension of sodium hydride in mineral oil (770 mg, 60% w/w, 19.25 mmol) in tetrahydrofuran (12 mL). The mixture was stirred at 40° C. for 90 min. The mixture was cooled to room temperature and a solution of methanesulfonyl chloride (2.22 g, 1.5 mL, 19.38 mmol) in tetrahydrofuran (12 mL) was added dropwise. The mixture was heated at 40° C. and stirred for 90 min. After cooling to room temperature, the mixture was quenched with water (20 mL) and saturated aqueous sodium bicarbonate solution (30 mL). The aqueous layer was extracted with ethyl acetate (2×50 mL). The combined organic layer was washed with brine, dried over anhydrous magnesium sulfate, filtered, and concentrated in vacuo. The crude product was partitioned between acetonitrile (100 mL) and heptanes (100 mL) and the heptane layer was extracted with acetonitrile (50 mL). The combined acetonitrile layers were concentrated under reduced pressure to give crude [1-(4-bromo-2,5-dimethyl-phenyl)-2,2,2-trifluoro-1-methyl-ethyl]methanesulfonate (2.75 g, 96%). ESI-MS m/z calc. 373.98, found 279.2 (M-95)⁺. ¹H NMR (400 MHz, CDCl₃) δ 7.43 (s, 1H), 7.27 (s, 1H), 3.17 (s, 3H), 2.55 (s, 3H), 2.39 (s, 3H), 2.35 (d, J=0.7 Hz, 3H) ppm. ¹⁹F NMR (377 MHz, CDCl₃) δ −79.08 (s, 3F) ppm.

### Step 4: 1-bromo-2,5-dimethyl-4-(2,2,2-trifluoro-1,1-dimethyl-ethyl)benzene

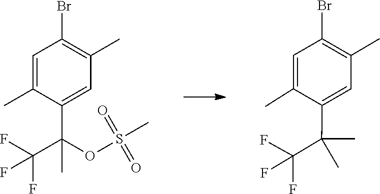

To a solution of [1-(4-bromo-2,5-dimethyl-phenyl)-2,2,2-trifluoro-1-methyl-ethyl]methanesulfonate (2.75 g, 6.08 mmol) in DCM (40 mL) cooled at 0° C. was added dropwise a solution of trimethylaluminum (2 M in hexanes) (8.2 mL of 2 M, 16.4 mmol) over a period of 5 min. The mixture was gradually warmed to room temperature and stirred for 2 h. The mixture was slowly quenched with saturated sodium bicarbonate solution (60 mL) and partitioned between brine (60 mL) and DCM (60 mL). The layers were separated, and aqueous layer was extracted with DCM (2×100 mL). The combined organic layers were washed with brine (50 mL), dried over anhydrous sodium sulfate, filtered, and concentrated under reduced pressure to give a yellow liquid. The crude product was purified by silica gel flash chromatography using heptanes to afford 1-bromo-2,5-dimethyl-4-(2,2,2-trifluoro-1,1-dimethyl-ethyl)benzene (1.48 g, 79%) as a yellow oil. ¹H NMR (400 MHz, CDCl₃) δ 7.35 (s, 1H), 7.31 (s, 1H), 2.50 (s, 3H), 2.38 (s. 3H), 1.67 (s, 6H) ppm. ¹⁹F NMR (377 MHz, CDCl₃) δ −75.18 (s, 3F) ppm.

### Step 5: 2-[2,5-dimethyl-4-(2,2,2-trifluoro-1,1-dimethyl-ethyl)phenyl]-4,4,5,5-tetramethyl-1,3,2-dioxaborolane (Intermediate B-3)

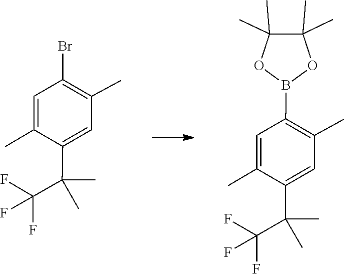 [SMILES: Cc1cc(C(C)(C)C(F)(F)F)c(C)cc1Br ; Cc1cc(C(C)(C)C(F)(F)F)c(C)cc1B1OC(C)(C)C(C)(C)O1]

2-[2,5-dimethyl-4-(2,2,2-trifluoro-1,1-dimethyl-ethyl)phenyl]-4,4,5,5-tetramethyl-1,3,2-dioxaborolane (Intermediate B-3) was prepared from 1-bromo-2,5-dimethyl-4-(2,2,2-trifluoro-1,1-dimethyl-ethyl)benzene using procedure analogous to that found in Intermediate B-1, Step 2. ESI-MS m/z calc. 342.19, found 343.2 (M+1)⁺. ¹H NMR (400 MHz, DMSO-d₆) δ 7.42 (s, 1H), 7.28 (s, 1H), 2.45 (s, 3H), 2.42 (s, 3H), 1.65 (s, 6H), 1.29 (s, 12H) ppm. ¹⁹F NMR (377 MHz, DMSO-d₆) δ −73.86 (s, 3F) ppm.

### Intermediate B-4

### 2-[5-chloro-2-methyl-4-(2,2,2-trifluoro-1,1-dimethyl-ethyl)phenyl]-4,4,5,5-tetramethyl-1,3,2-dioxaborolane

### Step 1: 1-bromo-5-chloro-2-methyl-4-(2,2,2-trifluoro-1,1-dimethyl-ethyl)benzene

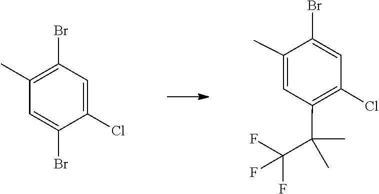

1-bromo-5-chloro-2-methyl-4-(2,2,2-trifluoro-1,1-dimethyl-ethyl)benzene was prepared from 1,4-dibromo-2-chloro-5-methyl-benzene using a procedure analogous to that found in Intermediate B-3 (Step 1 to Step 4). ¹H NMR (400 MHz, CDCl₃) δ 7.59 (s, 1H), 7.38 (s, 1H), 2.38 (s, 3H), 1.75 (s, 6H) ppm.

### Step 2: 2-[5-chloro-2-methyl-4-(2,2,2-trifluoro-1,1-dimethyl-ethyl)phenyl]-4,4,5,5-tetramethyl-1,3,2-dioxaborolane (Intermediate B-4)

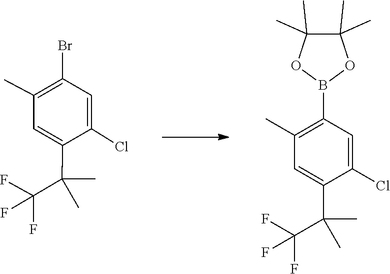 [SMILES: Cc1cc(C(C)(C)C(F)(F)F)c(Cl)cc1Br ; Cc1cc(C(C)(C)C(F)(F)F)c(Cl)cc1B1OC(C)(C)C(C)(C)O1]

2-[5-chloro-2-methyl-4-(2,2,2-trifluoro-1,1-dimethyl-ethyl)phenyl]-4,4,5,5-tetramethyl-1,3,2-dioxaborolane (Intermediate B-4) was prepared using procedure analogous to that found in Intermediate B-1, Step 2 as an off-white solid. ESI-MS m/z calc. 362.14, found 363.2 (M+1)⁺. ¹H NMR (400 MHz, CDCl₃) δ 7.77 (s, JH), 7.32 (s, 1H), 2.50 (s, 3H), 1.75 (s, 6H), 1.34 (s, 12H) ppm. 19F NMR (377 MHz, CDCl₃) δ −73.72 (s, 3F) ppm.

### Intermediate B-5

### 2-[4-(3,3-difluorocyclobutyl)-2-methyl-phenyl]-4,4,5,5-tetramethyl-1,3,2-dioxaborolane

### Step 1: 1-bromo-2-methyl-4-vinyl-benzene

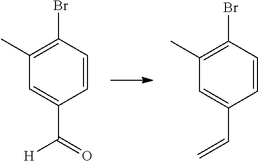

To a solution of methyl(triphenyl)phosphonium bromide (14.5 g, 40.59 mmol) in THF (100 mL) was added n-BuLi (16 mL of 2.5 M, 40 mmol) dropwise and stirred for 90 min under a stream of nitrogen at 0° C. 4-Bromo-3-methyl-benzaldehyde (5 g, 25.12 mmol) in THF (10 mL) was added dropwise at −20° C. and the mixture was stirred for 2 h. The mixture was warmed to room temperature and quenched with saturated ammonium chloride solution. The aqueous layer was extracted with ethyl acetate (3×). The combined organic layer was washed with brine, dried over magnesium sulfate, filtered and concentrated. Purification via silica gel column chromatography using 0 to 30% ethyl acetate in hexanes gave 1-bromo-2-methyl-4-vinyl-benzene (2.1 g, 42%). ¹H NMR (400 MHz, CDCl₃) δ 7.47 (d, J=8.2 Hz, JH), 7.25 (d, J=2.6 Hz, 1H), 7.09 (dd, J=8.2, 2.2 Hz, 1H), 6.63 (dd, J=17.6, 10.8 Hz, 1H), 5.73 (dd, J=17.6, 0.8 Hz, 1H), 5.25 (dd, J=10.9, 0.8 Hz, 1H), 2.39 (s, 3H) ppm.

### Step 2: 3-(4-bromo-3-methyl-phenyl)cyclobutanone

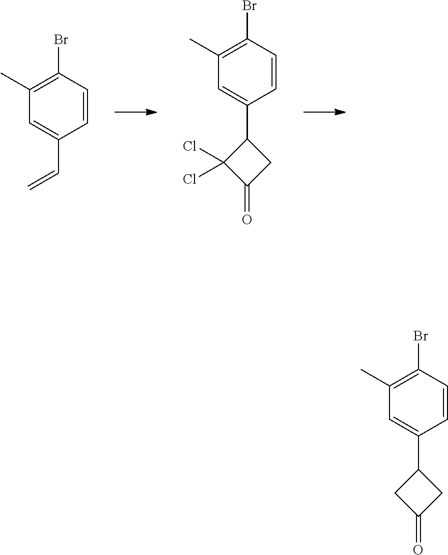

To a stirred suspension of activated copper-zinc (3.5 g, 27.14 mmol) and 1-bromo-2-methyl-4-vinyl-benzene (2.1 g, 10.66 mmol) in dry ether (30 mL) was added dropwise through an addition funnel, a solution of 2,2,2-trichloroacetyl chloride (2.4 mL, 21.50 mmol) and POCl₃ (2 mL, 21.46 mmol) in ether (15 mL). The suspension was stirred overnight at reflux. The mixture was cooled to room temperature and filtered through a pad of Celite®. The filtrate was quenched by slowly pouring into water. The layers were separated, and the organic layer was washed with sodium bicarbonate, dried over magnesium sulfate, filtered and concentrated in vacuo to obtain 3-(4-bromo-3-methyl-phenyl)-2,2-dichloro-cyclobutanone, which was dissolved in acetic acid (7 mL). Zinc (3.15 g, 48.16 mmol) was slowly added portion-wise, and the slurry was stirred at room temperature for 30 min followed by heating at 115° C. for 16 h. The mixture was cooled to room temperature, diluted with ethyl acetate, filtered through Celite®, and concentrated. The resulting oil was purified by silica gel chromatography by eluting with 0 to 25% ethyl acetate in heptanes followed by reverse phase purification (1-70% acetonitrile/water containing HCl modifier) to obtain 3-(4-bromo-3-methyl-phenyl)cyclobutanone (620 mg, 24%). ESI-MS m/z calc. 237.99, found 238.95 (M+1)⁺.

### Step 3: 1-bromo-4-(3,3-difluorocyclobutyl)-2-methyl-benzene

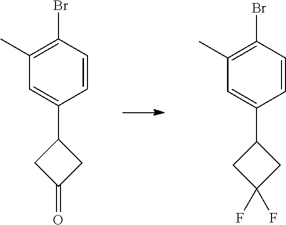

A solution of 3-(4-bromo-3-methyl-phenyl)cyclobutanone (620 mg, 2.59 mmol) in DCM (15 mL) was cooled to −70° C. (external temperature) and DAST (2 mL, 15.14 mmol) was added slowly. The mixture was gradually warmed to room temperature and stirred for 3 days. 1 N sodium hydroxide was added (20 mL) and the mixture was stirred vigorously for 30 min. DCM was added and the layers were separated. The organic layer was dried over magnesium sulfate, filtered, and concentrated. Purification via silica gel column chromatography using 0 to 10% ethyl acetate in hexanes afforded 1-bromo-4-(3,3-difluorocyclobutyl)-2-methyl-benzene (400 mg, 59%). ¹H NMR (400 MHz, CDCl₃) δ 7.48 (d, J=8.2 Hz, 1H), 7.09 (d, J=2.3 Hz, 1H), 6.92 (dd, J=8.2, 2.3 Hz, 1H), 3.38-3.24 (m, 1H), 3.05-2.92 (m, 2H), 2.71-2.55 (m, 2H), 2.39 (s, 3H) ppm.

### Step 4: 2-[4-(3,3-difluorocyclobutyl)-2-methyl-phenyl]-4,4,5,5-tetramethyl-1,3,2-dioxaborolane (Intermediate B-5)

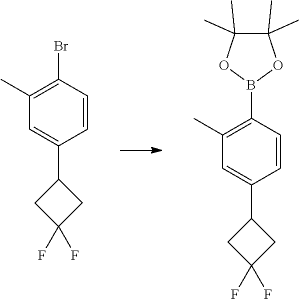

2-[4-(3,3-difluorocyclobutyl)-2-methyl-phenyl]-4,4,5,5-tetramethyl-1,3,2-dioxaborolane (Intermediate B-5) was prepared from 1-bromo-4-(3,3-difluorocyclobutyl)-2-methyl-benzene using a procedure analogous to that found in Intermediate B-1, Step 2. Pd(dppf)Cl₂·DCM was used as the catalyst. ESI-MS m/z calc. 308.18, found 309.17 (M+1)-. ¹H NMR (400 MHz, CDCl₃) δ 7.75-7.68 (m, 1H), 7.06-6.99 (m, 2H), 3.41-3.27 (m, 1H), 3.05-2.91 (m, 2H), 2.78-2.59 (m, 2H), 2.53 (s, 3H), 1.33 (s, 12H) ppm.

### Intermediate B-6

### 2-[4-(3,3-difluoro-1-methyl-cyclobutyl)-2-methyl-phenyl]-4,4,5,5-tetramethyl-1,3,2-dioxaborolane

### Step 1: 3-(4-bromo-3-methyl-phenyl)-2,2-dichloro-3-methyl-cyclobutanone and 3-(4-bromo-3-methyl-phenyl)-3-methyl-cyclobutanone

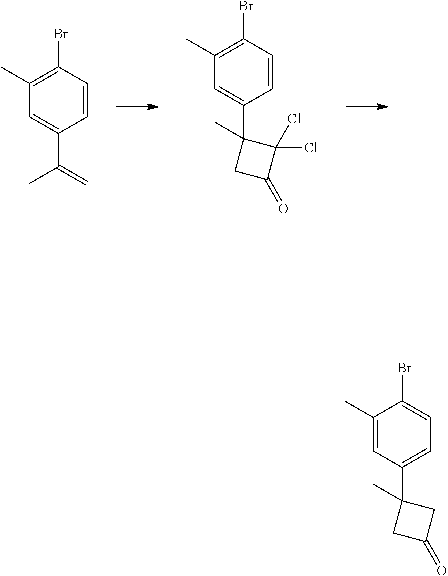 [SMILES: C=C(C)c1ccc(Br)c(C)c1 ; Cc1cc(C2(C)CC(=O)C2(Cl)Cl)ccc1Br ; Cc1cc(C2(C)CC(=O)C2)ccc1Br]

To a stirred suspension of activated copper-zinc (5.75 g, 44.8 mmol) and 1-bromo-4-isopropenyl-2-methyl-benzene (prepared from 1-(4-bromo-3-methyl-phenyl)ethanone via a process analogous to Intermediate B-5, Step 1, 3.68 g, 17.43 mmol) in dry Et₂O (50 mL) was added dropwise through an addition funnel, a solution of 2,2,2-trichloroacetyl chloride (4 mL, 35.84 mmol) and POCl₃ (3.3 mL, 35.40 mmol) in ether (25 mL). The suspension was stirred overnight at reflux. The mixture was cooled to room temperature and then filtered through a pad of Celite® and quenched by slowly pouring into water. The water layer was removed, and the remaining organic layer was washed with saturated sodium bicarbonate solution, dried over magnesium sulfate, filtered and concentrated in vacuo to obtain 3-(4-bromo-3-methyl-phenyl)-2,2-dichloro-3-methyl-cyclobutanone. It was dissolved in acetic acid (13 mL), then zinc (5.2 g, 79.50 mmol) was slowly added in small portions. The slurry was stirred at room temperature for 30 min then heated to 115° C. for 16 h. After that time, the reaction was cooled to room temperature, diluted with ethyl acetate, filtered through Celite®, and concentrated. The resulting oil was purified by silica gel chromatography by eluting with 0 to 20% heptane in ethyl acetate to obtain 3-(4-bromo-3-methyl-phenyl)-3-methyl-cyclobutanone (2.03 g, 46%). ESI-MS m/z calc. 252.015, found 254.98 (M+1)⁺. ¹H NMR (400 MHz, CDCl₃) δ 7.51 (d, J=8.2 Hz, 1H), 7.16 (d, J=2.5 Hz, 1H), 6.99 (dd, J=8.2, 2.5 Hz, 1H), 3.50-3.33 (m, 2H), 3.15-3.05 (m, 2H), 2.42 (s, 3H), 1.59 (s, 3H) ppm.

### Step 2: 1-bromo-4-(3,3-difluoro-1-methyl-cyclobutyl)-2-methyl-benzene

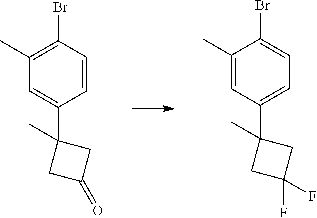

A solution of 3-(4-bromo-3-methyl-phenyl)-3-methyl-cyclobutanone (2 g, 7.901 mmol) in DCM (50 mL) was cooled to −70° C. (external temperature) and DAST (6 mL, 45.41 mmol) was added slowly. The mixture was gradually warmed to room temperature and stirred for 3 days. 1N NaOH was added (20 mL) and the mixture was stirred vigorously for 30 min. DCM was added and the layers were separated. The organic layer was dried over magnesium sulfate, filtered and concentrated. Purification via silica gel column chromatography using 0 to 10% ethyl acetate in hexanes gave 1-bromo-4-(3,3-difluoro-1-methyl-cyclobutyl)-2-methyl-benzene (515 mg, 24%). ¹H NMR (400 MHz, CDCl₃) δ 7.48 (d, J=8.2 Hz, 1H), 7.04 (d, J=2.5 Hz, 1H), 6.87 (dd, J=8.2, 2.5 Hz, 1H), 3.00-2.81 (m, 2H), 2.76-2.62 (m, 2H), 2.40 (s, 3H), 1.50 (d, J=1.3 Hz, 3H) ppm. ¹⁹F NMR (376 MHz, CDCl₃) δ −84.43 (dtt, J=195.9, 12.9, 6.2 Hz), −91.91 (dp, J=195.9, 15.2 Hz) ppm.

### Step 3: 2-[4-(3,3-difluoro-1-methyl-cyclobutyl)-2-methyl-phenyl]-4,4,5,5-tetramethyl-1,3,2-dioxaborolane (Intermediate B-6)

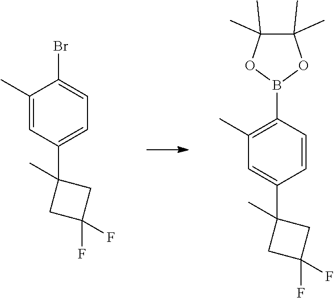 [SMILES: Cc1cc(C2(C)CC(F)(F)C2)ccc1Br ; Cc1cc(C2(C)CC(F)(F)C2)ccc1B1OC(C)(C)C(C)(C)O1]

2-[4-(3,3-difluoro-1-methyl-cyclobutyl)-2-methyl-phenyl]-4,4,5,5-tetramethyl-1,3,2-dioxaborolane (Intermediate B-6, 506 mg, 85%) was prepared using procedure analogues to that in Intermediate B-1, Step 2. ESI-MS m/z calc. 322.19, found 323.2 (M+1)⁺. ¹H NMR (400 MHz, CDCl₃) δ 7.78-7.72 (m, 1H), 7.02-6.96 (m, 2H), 3.00-2.84 (m, 2H), 2.76-2.61 (m, 2H), 2.54 (s, 3H), 1.53-1.48 (m, 3H), 1.33 (s, 12H) ppm. ¹⁹F NMR (376 MHz, CDCl₃) δ −83.95 (td, J=12.9, 6.5 Hz), −84.48 (tt, J=12.9, 5.8 Hz), −91.71 (p, J=15.6 Hz), −92.23 (p, J=15.6 Hz) ppm.

### Intermediate B-7

### 2-tert-butyl-3,6-dimethyl-5-(4,4,5,5-tetramethyl-1,3,2-dioxaborolan-2-yl)pyridine

### Step 1: sodium 4,4-dimethyl-1-oxo-pent-2-en-3-olate

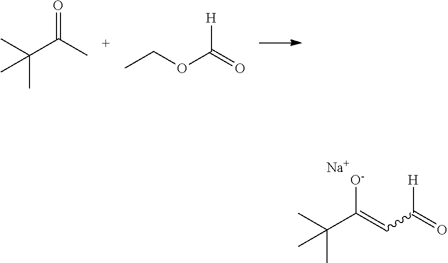

To a suspension of sodium hydride in mineral oil (8.4 g, 60% w/w, 210.02 mmol) in diethyl ether (120 mL) at 0° C. was added a solution of 3,3-dimethylbutan-2-one (20.83 g, 26 mL, 207.93 mmol) and ethyl formate (15.47 g, 16.8 mL, 208.87 mmol) in diethyl ether (30 mL) dropwise over 35 min. The mixture was gradually warmed to room temperature and stirred overnight. It was diluted with diethyl ether (250 mL) and stirred vigorously for 30 min, filtered, rinsed with diethyl ether (2×500 mL) and air dried to give a 3.7:1 crude mixture of Z/E isomer of sodium 4,4-dimethyl-1-oxo-pent-2-en-3-olate (16.05 g, 49%) as an off-white solid. Major isomer: ¹H NMR (400 MHz, DMSO-d₆) δ 9.05 (d, J=9.5 Hz, 1H), 4.77 (d, J=9.5 Hz, 1H), 0.94 (s, 9H) ppm. Minor isomer: ¹H NMR (400 MHz, DMSO-d₆) δ 8.21 (d, J=3.9 Hz, 1H), 4.82 (d, J=3.9 Hz, 1H), 1.01 (s, 9H) ppm.

### Step 2: 6-tert-butyl-2-hydroxy-pyridine-3-carbonitrile

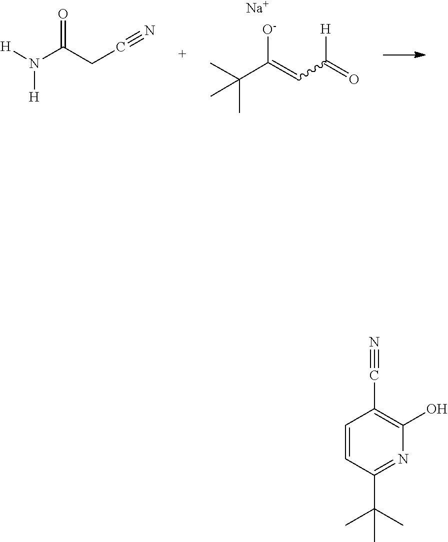

A solution of piperidine acetate (8.85 g, 60.95 mmol) in water (6.1 mL) was added to a solution of sodium 4,4-dimethyl-1-oxo-pent-2-en-3-olate (16.05 g, 101.55 mmol) and 2-cyanoacetamide (8.54 g, 101.57 mmol) in water (85 mL) at room temperature. The solution was stirred at reflux for 5 h. The mixture was cooled to room temperature and acidified (pH 4) with glacial acetic acid. The resulting precipitate was filtered, rinsed with water (3×500 mL) and dried under high vacuum to give 6-tert-butyl-2-hydroxy-pyridine-3-carbonitrile (10.9 g, 61%) as a light-yellow solid. ESI-MS m/z calc. 176.09. found 177.2 (M+1)⁺. ¹H NMR (400 MHz, DMSO-d₆) δ 12.18 (br s, 1H), 8.06 (d, J=7.6 Hz, 1H), 6.24 (br d, J=7.3 Hz, 1H), 1.27 (s, 9H) ppm. ESI-MS m/z calc. 176.09, found 177.2 (M+1)⁺.

### Step 3: 5-bromo-6-tert-butyl-2-hydroxy-pyridine-3-carbonitrile

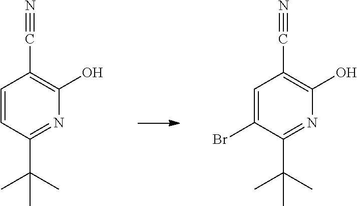

A solution of 6-tert-butyl-2-hydroxy-pyridine-3-carbonitrile (430 mg, 2.44 mmol) and NBS (651 mg, 3.66 mmol) in anhydrous 1,2-dichloroethane (5 mL) was stirred at reflux for 2.5 h. After it was cooled to room temperature, water (10 mL) was added, and the aqueous layer was extracted with DCM (2×10 mL). The combined organic layer was washed with water (15 mL), dried over sodium sulfate, filtered, and concentrated under reduced pressure. The crude product was purified by silica gel flash chromatography using 0 to 10% methanol in DCM to afford 5-bromo-6-tert-butyl-2-hydroxy-pyridine-3-carbonitrile (480 mg, 77%) as a yellow oil. ESI-MS m/z calc. 254.0, found 255.0 (M+1)⁺. ¹H NMR (400 MHz, DMSO-d₆) δ 8.37 (s, 1H), 1.45 (s, 9H) ppm.

### Step 4: 2,5-dibromo-6-tert-butyl-pyridine-3-carbonitrile

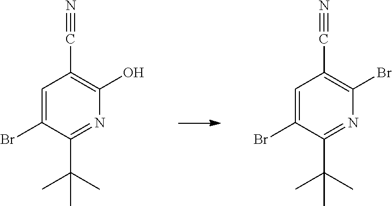

To a stirred suspension of 5-bromo-6-tert-butyl-2-hydroxy-pyridine-3-carbonitrile (16.11 g, 61.76 mmol) in toluene (200 mL) was added phosphorus oxybromide (26.6 g, 92.79 mmol). The mixture was stirred at 95° C. overnight. The mixture was cooled to room temperature and quenched with water (800 mL) and diluted with ethyl acetate (500 mL) and brine (200 mL). The layers were separated, and the aqueous layer was extracted with ethyl acetate (2×400 mL). The combined organic layer was washed with brine (400 mL), dried over sodium sulfate, filtered, and concentrated under reduced pressure. The crude product was purified by silica gel flash chromatography using 0 to 10% ethyl acetate in heptanes to afford 2,5-dibromo-6-tert-butyl-pyridine-3-carbonitrile (15.44 g, 79%) as an orange solid. ESI-MS m/z calc. 254.01, found 255.0 (M+1)⁺.

### Step 5: 6-tert-butyl-2,5-dimethyl-pyridine-3-carbonitrile

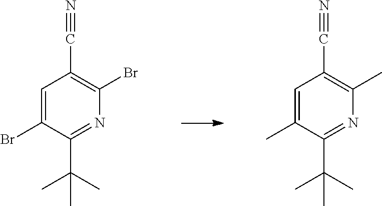

A suspension of 2,5-dibromo-6-tert-butyl-pyridine-3-carbonitrile (2 g, 6.28 mmol), trimethylboroxine (2.42 g, 2.7 mL, 19.31 mmol) and potassium carbonate (5.21 g, 37.7 mmol) in anhydrous 1,4-dioxane (20 mL) was purged with nitrogen for 10 min, Pd(dppf)₂Cl₂·DCM (515 mg, 0.63 mmol) was added to the mixture and it was purged with nitrogen for an additional 10 min. The mixture was heated at 100° C. and stirred overnight. It was cooled to room temperature, filtered through Celite®, rinsed with methanol (150 mL) and the filtrate was concentrated under reduced pressure. The crude product was purified by reverse phase chromatography (C₁₈, 0-100% acetonitrile/water, containing 0.1% formic acid) to afford 6-tert-butyl-2,5-dimethyl-pyridine-3-carbonitrile (841 mg, 71%). ESI-MS m/z calc. 188.13, found 189.2 (M+1)⁺. ¹H NMR (400 MHz, DMSO-d₆) δ 7.93 (s, 1H), 2.58 (s, 3H), 2.47 (s, 3H), 1.37 (s, 9H) ppm.

### Step 6: 6-tert-butyl-2,5-dimethyl-pyridine-3-carboxylic acid

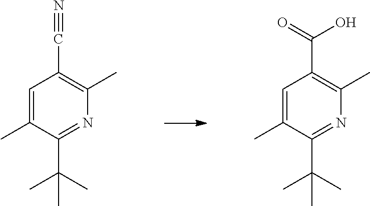

To a solution of 6-tert-butyl-2,5-dimethyl-pyridine-3-carbonitrile (840 mg, 4.46 mmol) in ethanol (10 mL) was added an aqueous solution of NaOH (5 mL of 10 M, 50 mmol). The mixture was heated at 100° C. and stirred overnight. The mixture was cooled to room temperature and ethanol was removed under reduced pressure. An aqueous solution of 6 M HCl was added (pH 7). The aqueous mixture was diluted with water (150 mL) and it was extracted with 2-MeTHF (10×150 mL). The combined organic layers were dried over sodium sulfate, filtered, and concentrated under reduced pressure. The crude product was purified by reverse phase chromatography (C18, 5-45% acetonitrile/water, containing 0.1% formic acid) to afford 6-tert-butyl-2,5-dimethyl-pyridine-3-carboxylic acid (851 mg, 92%) as a white solid. ESI-MS m/z calc. 207.13, found 208.2 (M+1)⁺. ¹H NMR (400 MHz, DMSO-d₆) δ 12.97 (br s, 1H), 7.87 (s, 1H), 2.64 (s, 3H), 2.47 (s, 3H), 1.37 (s, 9H) ppm.

### Step 7: 5-bromo-2-tert-butyl-3,6-dimethyl-pyridine

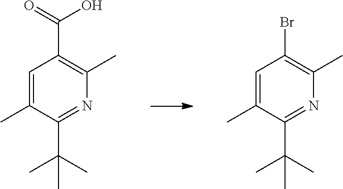

A suspension of 6-tert-butyl-2,5-dimethyl-pyridine-3-carboxylic acid (5.62 g, 27.09 mmol), tetrabutylammonium tribromide (39.9 g, 82.75 mmol) and potassium phosphate (5.86 g, 27.61 mmol) in acetonitrile (120 mL) was purged with nitrogen for 20 min. The mixture was heated at 100° C. and stirred for 48 h followed by stirring for 72 h at room temperature. The mixture was filtered, rinsed with acetonitrile (200 mL) and concentrated under reduced pressure. The crude product was purified by silica gel flash chromatography using 0 to 5% ethyl acetate in heptanes to afford 5-bromo-2-tert-butyl-3,6-dimethyl-pyridine (3.91 g, 59%) as a light-yellow oil. ESI-MS m/z calc. 241.05, found 242.1 (M+1)⁺. ¹H NMR (400 MHz, CDCl₃) δ 7.47 (s, 1H), 2.57 (s, 3H), 2.44 (s, 3H), 1.40 (s, 9H) ppm.

### Step 8: 2-tert-butyl-3,6-dimethyl-5-(4,4,5,5-tetramethyl-1,3,2-dioxaborolan-2-yl)pyridine (Intermediate B-7)

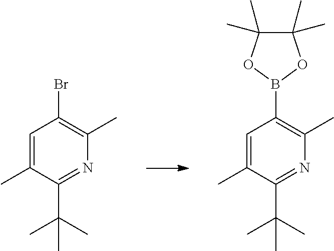

2-tert-butyl-3,6-dimethyl-5-(4,4,5,5-tetramethyl-1,3,2-dioxaborolan-2-yl)pyridine (Intermediate B-7) was prepared from 5-bromo-2-tert-butyl-3,6-dimethyl-pyridine using a procedure analogous to that found in Intermediate B-1, Step 2. ¹H NMR (400 MHz, CDCl₃) δ 7.68 (s, 1H), 2.66 (s, 3H), 2.45 (s, 3H), 1.41 (s, 9H), 1.34 (s, 12H) ppm.

### Intermediate B-8

### 2-tert-butyl-3-chloro-6-methyl-5-(4,4,5,5-tetramethyl-1,3,2-dioxaborolan-2-yl)pyridine

### Step 1: 6-tert-butyl-5-chloro-2-hydroxy-pyridine-3-carbonitrile

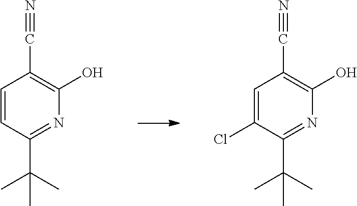

A solution of 6-tert-butyl-2-hydroxy-pyridine-3-carbonitrile (5 g, 28.35 mmol) and NCS (4.7 g, 35.2 mmol) in anhydrous 1,2-dichloroethane (25 mL) was stirred at 80° C. for 4 h Once cooled to room temperature, the mixture was treated with saturated aqueous sodium bicarbonate solution (100 mL) It was diluted with water (100 mL) and the aqueous layer was extracted with DCM (2×300 mL). The combined organic layer was washed with water (200 mL), brine (200 mL), dried over sodium sulfate, filtered and concentrated under reduced pressure to give 6-tert-butyl-5-chloro-2-hydroxy-pyridine-3-carbonitrile (5.99 g, 100%) as a beige solid. ESI-MS m/z calc. 210.06, found 211.2 (M+1)⁺. ¹H NMR (400 MHz, CDCl₃) δ 9.94 (br s, 1H), 7.78 (s, 1H), 1.53 (s, 9H) ppm.

### Step 2: 2-bromo-6-tert-butyl-5-chloro-pyridine-3-carbonitrile

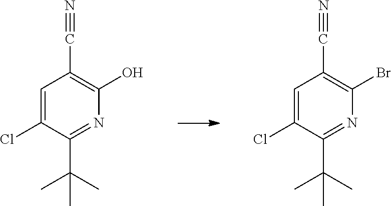

To a stirred suspension of 6-tert-butyl-5-chloro-2-hydroxy-pyridine-3-carbonitrile (5.9 g, 27.98 mmol) in toluene (90 mL) was added phosphorus oxybromide (11 g, 38.37 mmol). The mixture was stirred at 95° C. for 16 h After the mixture was cooled to room temperature, it was quenched by slow addition of saturated aqueous sodium bicarbonate solution (150 mL). The mixture was poured in a separatory funnel and diluted with water (200 mL). The aqueous layer was extracted with ethyl acetate (2×200 mL). The combined organic layer was washed with brine (100 mL), dried over sodium sulfate, filtered and concentrated under reduced pressure. The crude product was purified via silica gel column chromatography using 0 to 10% ethyl acetate in heptane to give 2-bromo-6-tert-butyl-5-chloro-pyridine-3-carbonitrile (6.29 g, 82%) as a dark orange oil. ESI-MS m/z calc. 271.97, found 273.0 (M+1)⁺. ¹H NMR (400 MHz, CDCl₃) δ 7.81 (s, 1H), 1.48 (s, 9H) ppm.

### Step 3: 6-tert-butyl-5-chloro-2-methyl-pyridine-3-carbonitrile

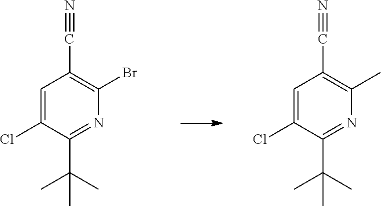

A solution of 2-bromo-6-tert-butyl-5-chloro-pyridine-3-carbonitrile (3.85 g, 14.1 mmol) in 1,4-dioxane (50 mL) was sparged with nitrogen for 10 min then 2,4,6-trimethyl-1,3,5,2,4,6-trioxatriborinane (1.8 g, 2 mL, 14.3 mmol), potassium carbonate (5.8 g, 42 mmol) and Pd(dppf)Cl₂·DCM (580 mg, 0.710 mmol) were added. The mixture was stirred for 4 h at 100° C. The solvent was removed under reduced pressure. The residue was adsorbed on silica gel under vacuum and purified by silica gel chromatography (80 g silica, 5-70% ethyl acetate/heptane) to provide a mixture of 6-tert-butyl-5-chloro-2-methyl-pyridine-3-carbonitrile and the 2-bromo-6-tert-butyl-5-chloro-pyridine-3-carbonitrile starting material. The mixture was resubjected to the above reaction conditions and purification to provide 6-tert-butyl-5-chloro-2-methyl-pyridine-3-carbonitrile. ESI-MS m/z calc. 208.07, found 209.2 (M+1)⁺.

### Step 4: 6-tert-butyl-5-chloro-2-methyl-pyridine-3-carboxylic acid

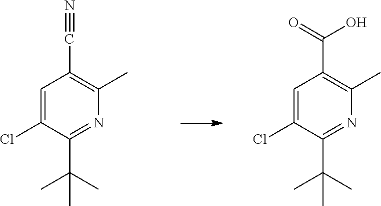

To a solution of 6-tert-butyl-5-chloro-2-methyl-pyridine-3-carbonitrile (2.09 g, 10 mmol) in ethanol (32 mL) was added aqueous NaOH (15 mL of 10 M, 150 nmol). The pale-yellow solution was stirred at 100° C. in a sealed tube for 24 h. The solvent was removed under reduced pressure and the aqueous residue was diluted with water (100 mL). An insoluble white solid precipitated formed which was removed by filtration and rinsed with water (50 mL). The filtrate was washed with MTBE (2×50 mL). The pH was adjusted to ˜4 by addition of aqueous 3M HCl (˜15 mL) and aqueous layer was extracted with DCM (3×100 mL). The combined organic layer was dried over anhydrous sodium sulfate, filtered and concentrated under reduced pressure to give crude 6-tert-butyl-5-chloro-2-methyl-pyridine-3-carboxylic acid (2.13 g, 93%) as a white solid). ESI-MS m/z calc. 227.07, found 228.2 (M+1)-. ¹H NMR (400 MHz, CDCl₃) δ 8.23 (s, 1H), 2.82 (s, 3H), 1.50 (s, 9H) ppm.

### Step 5: 5-bromo-2-tert-butyl-3-chloro-6-methyl-pyridine

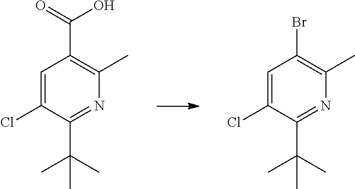

A flame-dried round-bottom flask was charged with 6-tert-butyl-5-chloro-2-methyl-pyridine-3-carboxylic acid (2.02 g, 8.86 mmol), potassium phosphate tribasic (3.7 g, 17.43 mmol), tetrabutylammonium tribromide (19 g, 39.41 mmol) and anhydrous acetonitrile (40 mL). The resulting mixture was refluxed at 100° C. for 95 h. The mixture was cooled to room temperature and was concentrated under reduced pressure. The residue was purified by silica gel column chromatography using 0 to 15% ethyl acetate in heptanes to give 5-bromo-2-tert-butyl-3-chloro-6-methyl-pyridine (1.24 g, 53%) as a colorless oil. ¹H NMR (400 MHz, CDCl₃) δ 7.72 (s, 1H), 2.58 (s, 3H), 1.45 (s, 9H) ppm.

### Step 6: 2-tert-butyl-3-chloro-6-methyl-5-(4,4,5,5-tetramethyl-1,3,2-dioxaborolan-2-yl)pyridine (Intermediate B-8)

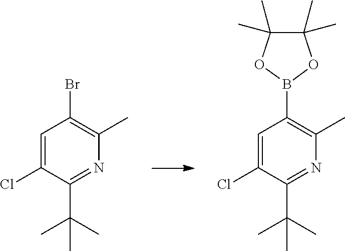 [SMILES: Cc1nc(C(C)(C)C)c(Cl)cc1Br ; Cc1nc(C(C)(C)C)c(Cl)cc1B1OC(C)(C)C(C)(C)O1]

2-tert-butyl-3-chloro-6-methyl-5-(4,4,5,5-tetramethyl-1,3,2-dioxaborolan-2-yl)pyridine (Intermediate B-8) was prepared from 5-bromo-2-tert-butyl-3-chloro-6-methyl-pyridine using a procedure analogous to that found in Intermediate B-1, Step 2 as a white solid. ESI-MS m/z calc. 309.17, found 310.1 (M+1)⁺. ¹H NMR (400 MHz, CDCl₃) δ 7.89 (s, 1H), 2.65 (s, 3H), 1.47 (s, 9H), 1.33 (s, 12H) ppm.

### Intermediate B-9

### tert-butyl-[2-[5-tert-butyl-2-(4,4,5,5-tetramethyl-1,3,2-dioxaborolan-2-yl)phenoxy]ethoxy]-dimethyl-silane

### Step 1: 2-(2-bromo-5-tert-butyl-phenoxy)ethoxy-tert-butyl-dimethyl-silane

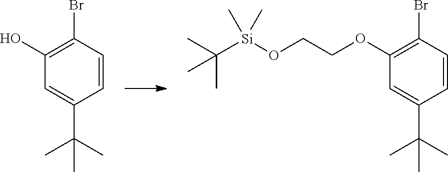 [SMILES: CC(C)(C)c1ccc(Br)c(O)c1 ; CC(C)(C)c1ccc(Br)c(OCCO[Si](C)(C)C(C)(C)C)c1]

To a 20 mL vial charged with 2-bromoethoxy-tert-butyl-dimethyl-silane (95 mg, 0.4 mmol) was added 2-bromo-5-tert-butyl-phenol (74 mg, 0.32 mmol), potassium carbonate (106 mg, 0.77 mmol) and DMF (2 mL). The vial was capped, and the mixture stirred at 70° C. for 16 h. After cooling to room temperature the crude mixture was diluted with DCM (˜40 mL), washed with water (˜10 mL), brine (˜10 mL), the organic layer dried over sodium sulfate, filtered and concentrated in vacuo. Purification by silica gel column chromatography using 0 to 30% ethyl acetate in hexanes afforded 2-(2-bromo-5-tert-butyl-phenoxy)ethoxy-tert-butyl-dimethyl-silane (104 mg, 83%). ESI-MS m/z calc. 388.13, found 389.4 (M+1)⁺.

### Step 2: tert-butyl-[2-[5-tert-butyl-2-(4,4,5,5-tetramethyl-1,3,2-dioxaborolan-2-yl)phenoxy]ethoxy]-dimethyl-silane (Intermediate B-9)

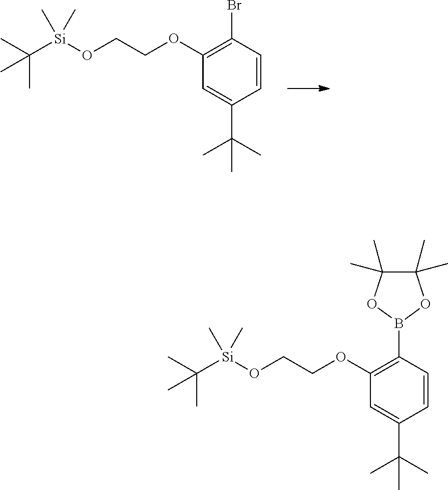 [SMILES: CC(C)(C)c1ccc(Br)c(OCCO[Si](C)(C)C(C)(C)C)c1 ; CC(C)(C)c1ccc(B2OC(C)(C)C(C)(C)O2)c(OCCO[Si](C)(C)C(C)(C)C)c1]

tert-butyl-[2-[5-tert-butyl-2-(4,4,5,5-tetramethyl-1,3,2-dioxaborolan-2-yl)phenoxy]ethoxy]-dimethyl-silane (Intermediate B-9) was synthesized using procedure analogous to that in Intermediate B-1, Step 2. It was subsequently used without purification.

### Intermediate B-10

### 3-methyl-5-[3-methyl-4-(4,4,5,5-tetramethyl-1,3,2-dioxaborolan-2-yl)phenyl]isoxazole

### Step 1: 5-(4-bromo-3-methyl-phenyl)-3-methyl-isoxazole

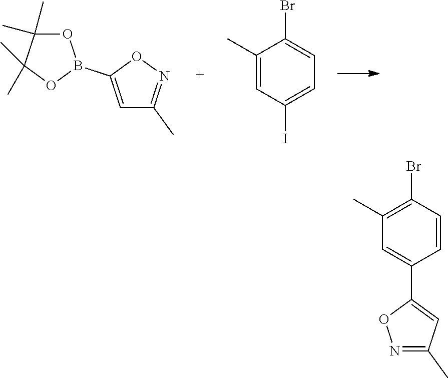

To a 20 mL vial charged with 1-bromo-4-iodo-2-methyl-benzene (590 mg, 2 mmol) was added 3-methyl-5-(4,4,5,5-tetramethyl-1,3,2-dioxaborolan-2-yl)isoxazole (201 mg, 0.96 mmol), Pd(dppf)₂Cl₂·DCM (65 mg, 0.08 mmol), potassium carbonate (402 mg, 2.9 mmol), 1,4-dioxane (5 mL), and water (1 mL). The reaction was purged with nitrogen for 30 seconds, capped and the mixture stirred at 50° C. for 18 h. After cooling to room temperature, water (˜10 mL) was added. The aqueous layer was extracted with DCM (3×25 mL). The combined organic layer was dried over sodium sulfate, filtered and concentrated in vacuo. Purification by silica gel column chromatography using 0 to 10% ethyl acetate in hexanes afforded 5-(4-bromo-3-methyl-phenyl)-3-methyl-isoxazole (34 mg, 14%). ESI-MS m/z calc. 250.99, found 252.2 (M+1)⁺.

### Step 2: 3-methyl-5-[3-methyl-4-(4,4,5,5-tetramethyl-1,3,2-dioxaborolan-2-yl)phenyl]isoxazole (Intermediate B-10)

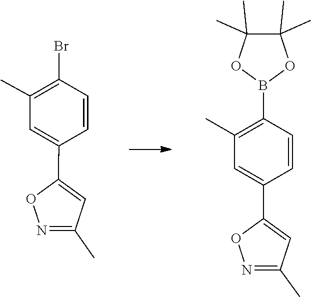

3-methyl-5-[3-methyl-4-(4,4,5,5-tetramethyl-1,3,2-dioxaborolan-2-yl)phenyl]isoxazole (Intermediate B-10) was prepared using procedure analogous to Intermediate B-1, Step 2. Intermediate B-10 was used without further purification. ESI-MS m/z calc. 299.17, found 300.5 (M+1)⁺.

### Intermediate B-11

### 2-(4-tert-butyl-2,6-dimethyl-phenyl)-4,4,5,5-tetramethyl-1,3,2-dioxaborolane

### Step 1: 2-bromo-5-tert-butyl-1,3-dimethyl-benzene

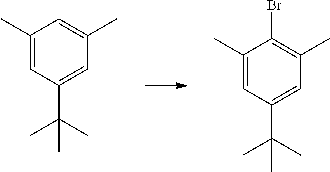

Under an inert atmosphere, acetic acid m was placed in a as and 1-tert-butyl-3,5-dimethyl-benzene (3 g, 18.49 mmol) was added, followed by the addition of bromine (1.25 mL, 24.26 mmol). The mixture was stirred at ambient temperature for 3 h and water (500 mL) was added. The product crashed out, which was filtered and washed with water (2×250 mL). Purification via silica gel column chromatography using 0 to 30% ethyl acetate in hexanes gave 2-bromo-5-tert-butyl-1,3-dimethyl-benzene (3.97 g, 89%). ¹H NMR (500 MHz, DMSO-d₆) δ 7.19 (s, 2H), 2.35 (s, 6H), 1.26 (s, 9H) ppm.

### Step 2: 2-(4-tert-butyl-2,6-dimethyl-phenyl)-4,4,5,5-tetramethyl-1,3,2-dioxaborolane (Intermediate B-11)

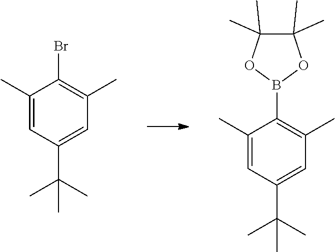

2-(4-tert-butyl-2,6-dimethyl-phenyl)-4,4,5,5-tetramethyl-1,3,2-dioxaborolane (Intermediate B-11) was prepared using a procedure analogous to Intermediate B-1, Step 2. ¹H NMR (500 MHz, DMSO-d₆) δ 6.96 (s, 2H), 2.31 (s, 6H), 1.32 (s, 12H), 1.24 (s, 9H) ppm.

### Intermediate B-12

### 2-(4-tert-butyl-2,5-dimethyl-phenyl)-4,4,5,5-tetramethyl-1,3,2-dioxaborolane

### Step 1: (4-tert-butyl-2,5-dimethyl-phenyl) trifluoromethanesulfonate and 2-(4-tert-butyl-2,5-dimethyl-phenyl)-4,4,5,5-tetramethyl-1,3,2-dioxaborolane (Intermediate B-12)

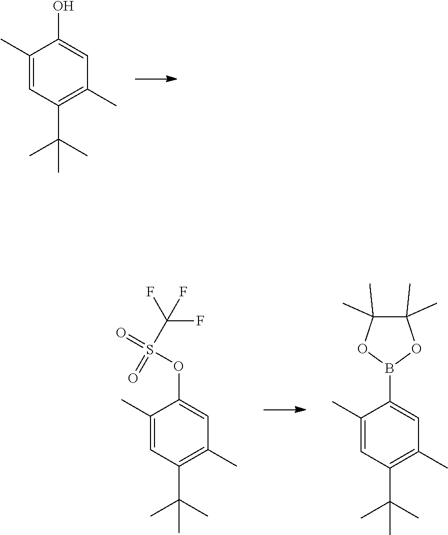

To a solution of 4-tert-butyl-2,5-dimethyl-phenol (2.568 g, 14.41 mmol) in DCM (29 mL) was added pyridine (2.33 mL, 28.81 mmol) and the mixture was cooled to 0° C. Trifluoromethanesulfonate (2.9 mL, 17.24 mmol) was added dropwise, and the reaction was gradually warmed to room temperature. After stirring at room temperature for 1.5 h, the mixture was diluted with ether and washed with 1N HCl. The organic layer was further washed with saturated sodium bicarbonate solution (3×) and brine. The organic layer was isolated, dried over magnesium sulfate, filtered, and evaporated to dryness. The crude material was purified by silica gel column chromatography using 0 to 10% ethyl acetate in hexanes. The intermediate triflate was recovered as a clear oil and used for the next step without further purification.

In a reaction vial, the intermediate from Step 1, (4-tert-butyl-2,5-dimethyl-phenyl)trifluoromethanesulfonate (4.223 g), was mixed with triethylamine (5.7 mL, 40.82 mmol) and pinacolborane (5.9 mL, 40.83 mmol) in 1,4-dioxane (68 mL). The mixture was purged with nitrogen and Pd(dppf)Cl₂ (300 mg, 0.41 mmol) was added. The mixture was refluxed overnight. The reaction was quenched with water and extracted with ethyl acetate. The layers were separated, and the organic layer was washed with brine, dried over sodium sulfate, filtered, and concentrated. Purification by silica gel column chromatography using 5 to 20% ethyl acetate in hexanes gave 2-(4-tert-butyl-2,5-dimethyl-phenyl)-4,4,5,5-tetramethyl-1,3,2-dioxaborolane (Intermediate B-12, 2.31 g, 56%) as a white solid. ¹H NMR (400 MHz, CDCl₃) δ 7.52 (s, 1H), 7.17 (s, 1H), 2.51 (s, 3H), 2.50 (s, 3H), 1.39 (s, 9H), 1.32 (s, 12H) ppm.

### Intermediate B-13

### 4,4,5,5-tetramethyl-2-[2-methyl-4-(2,2,2-trifluoro-1,1-dimethyl-ethyl)phenyl]-1,3,2-dioxaborolane

### Step 1: 1,1,1-trifluoro-2-(4-methoxy-3-methyl-phenyl)propan-2-ol

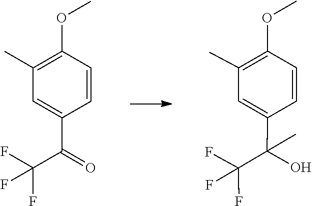 [SMILES: COc1ccc(C(=O)C(F)(F)F)cc1C ; COc1ccc(C(C)(O)C(F)(F)F)cc1C]

To a solution of 2,2,2-trifluoro-1-(4-methoxy-3-methyl-phenyl)ethanone (1.7 g, 7.79 mmol) in tetrahydrofuran (28 mL) cooled to 0° C. was slowly added methylmagnesium bromide (7.8 mL of 3 M, 23.4 mmol) as a solution in diethyl ether and the mixture was stirred at 50° C. for 2 h. The mixture was cooled to 0° C. and quenched slowly with water and a saturated solution of ammonium chloride. The layers were separated, and the aqueous layer was extracted with ethyl acetate (3×25 mL). The combined organic layer was washed with brine, dried over anhydrous magnesium sulfate, filtered, concentrated in vacuo and dried under high vacuum to provide 1,1,1-trifluoro-2-(4-methoxy-3-methyl-phenyl)propan-2-ol (1.81 g, 98%) as a light orange oil ESI-MS m/z calc. 234.09, found 216.4 (M-189). ¹H NMR (400 MHz, DMSO-d₆) δ 7.37 (s, 1H), 7.35 (s, 1H), 6.92 (d, J=8.3 Hz, 1H), 6.40 (s, 1H), 3.78 (s, 3H), 2.16 (s, 3H), 1.64 (s, 3H) ppm. ¹⁹F NMR (377 MHz, DMSO-d₆) δ −79.85 (s, 3F) ppm.

### Step 2: 1-methoxy-2-methyl-4-(2,2,2-trifluoro-1,1-dimethyl-ethyl)benzene

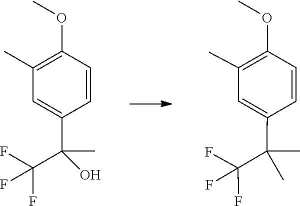 [SMILES: COc1ccc(C(C)(O)C(F)(F)F)cc1C ; COc1ccc(C(C)(C)C(F)(F)F)cc1C]

To a solution of 1,1,1-trifluoro-2-(4-methoxy-3-methyl-phenyl)propan-2-ol (2.7 g, 11.53 mmol) in DCM (108 mL) cooled to 0° C. was added a solution of titanium(IV) chloride (11.9 mL of 1 M, 11.9 mmol) in toluene and the mixture was stirred at the same temperature for 2 h. Ice-cold water and DCM were added, and the layers were separated. The aqueous layer was extracted with DCM (2×100 mL). The combined organic layer was washed with a saturated solution of sodium bicarbonate, dried over anhydrous magnesium sulfate, filtered and concentrated in vacuo. The residue was dissolved in DCM (108 mL) and cooled to −70° C. and a solution of titanium(IV) chloride (11.9 mL of 1 M, 11.9 mmol) in toluene was added dropwise, followed by dropwise addition of a solution of dimethylzinc (9.8 mL of 2 M, 19.6 mmol) in toluene. The mixture was gradually wanned to room temperature and stirred for 68 h. Ice-cold water and DCM were added, and the mixture was filtered over Celite®. The layers were separated, and the aqueous layer was extracted with DCM (2×50 mL). The combined organic layer was washed with a saturated solution of sodium bicarbonate, dried over anhydrous magnesium sulfate, filtered, concentrated in vacuo and dried under high vacuum to provide crude 1-methoxy-2-methyl-4-(2,2,2-trifluoro-1,1-dimethyl-ethyl)benzene (2.55 g, 44%) as an orange oil. ¹H NMR (400 MHz, CDCl₃) δ 7.32-7.24 (m, 2H), 6.84-6.78 (d, J=8.8 Hz, 1H), 3.84 (s, 3H), 2.24 (s, 3H), 1.56 (s, 6H) ppm. ¹⁹F NMR (377 MHz, CDCl₃) δ −76.41 (s, 3F) ppm.

### Step 3: 2-methyl-4-(2,2,2-trifluoro-1,1-dimethyl-ethyl)phenol

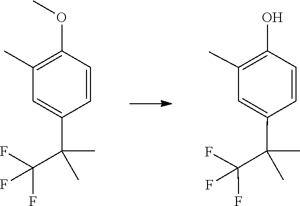 [SMILES: COc1ccc(C(C)(C)C(F)(F)F)cc1C ; Cc1cc(C(C)(C)C(F)(F)F)ccc1O]

To a solution of 1-methoxy-2-methyl-4-(2,2,2-trifluoro-1,1-dimethyl-ethyl)benzene (2.18 g, 5.39 mmol) in DCM (34 mL) cooled to 0° C. was added a solution of boron tribromide (5.4 mL of 1 M, 5.4 mmol) in DCM dropwise. The mixture was warmed to room temperature and stirred for 1 h. The solution was cooled again to 0° C. and an additional solution of boron tribromide (5.4 mL of 1 M, 5.4 mmol) in DCM was added. After stirring for 16 h at room temperature, the mixture was again cooled to 0° C., and a solution of boron tribromide (2.7 mL of 1 M, 2.7 mmol) in DCM was added. The mixture was warmed to room temperature and stirred for 0.5 h. The mixture was cooled down to 0° C. and quenched by slow addition of water (50 mL). The layers were separated, and the aqueous layer was extracted with DCM (3×50 mL). The combined organic layer was washed with brine, dried over anhydrous magnesium sulfate and concentrated in vacuo. Purification by silica gel chromatography using 0 to 5% ethyl acetate in heptanes followed by a second purification using reverse phase chromatography (C18, 5-100% methanol/water, containing 0.1% formic acid) provided 2-methyl-4-(2,2,2-trifluoro-1,1-dimethyl-ethyl)phenol (1.21 g, 99%) as a brown oil. ESI-MS m/z calc. 218.09, found 219.1 (M+1)⁺. ¹H NMR (400 MHz, CDCl₃) δ 7.27-7.17 (m, 2H), 6.76 (d, J=8.3 Hz, 1H), 4.84 (br. s, 1H), 2.28 (s, 3H), 1.55 (s, 6H) ppm. ¹⁹F NMR (377 MHz, CDCl₃) δ −76.43 (s, 3F) ppm.

### Step 4: [2-methyl-4-(2,2,2-trifluoro-1,1-dimethyl-ethyl)phenyl]trifluoromethanesulfonate

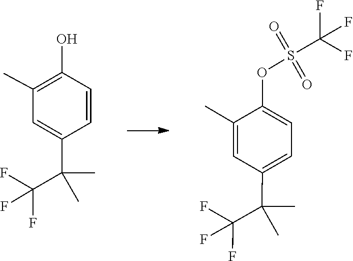 [SMILES: Cc1cc(C(C)(C)C(F)(F)F)ccc1O ; Cc1cc(C(C)(C)C(F)(F)F)ccc1OS(=O)(=O)C(F)(F)F]

To a solution of 2-methyl-4-(2,2,2-trifluoro-1,1-dimethyl-ethyl)phenol (1.21 g, 5.35 mmol) and pyridine (850.86 mg, 0.87 mL, 10.76 mmol) in DCM (25 mL) cooled at −50° C. under an atmosphere of nitrogen was added trifluoromethanesulfonic anhydride (2 g, 1.2 mL, 7.1 mmol) dropwise. The mixture was warmed to room temperature over 1 h. The mixture was washed with water (25 mL) and 1N aqueous HCl (25 mL), dried over anhydrous magnesium sulfate, filtered and concentrated in vacuo. Purification by silica gel chromatography using 0 to 20% ethyl acetate in heptanes provided [2-methyl-4-(2,2,2-trifluoro-1,1-dimethyl-ethyl)phenyl]trifluoromethanesulfonate (1.68 g, 89%) as a yellow oil. ¹H NMR (400 MHz, CDCl₃) δ 7.44-7.36 (m, 2H), 7.23 (d, J=8.6 Hz, 1H), 2.41 (s, 3H), 1.58 (s, 6H) ppm. 19F NMR (377 MHz, CDCl₃) δ −73.85 (s, 3F), −76.15 (s, 3F) ppm.

### Step 5: 4,4,5,5-tetramethyl-2-[2-methyl-4-(2,2,2-trifluoro-1,1-dimethyl-ethyl)phenyl]-1,3,2-dioxaborolane (Intermediate B-13)

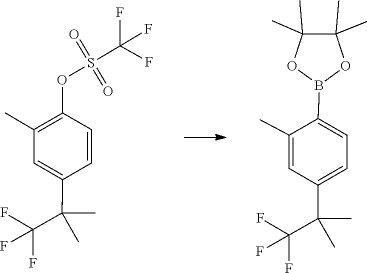

To a solution of [2-methyl-4-(2,2,2-trifluoro-1,1-dimethyl-ethyl)phenyl]trifluoromethanesulfonate (5.94 g, 16.941 mmol) in 1,4-dioxane (35 mL) were added bis(pinacolato)diboron (5.2 g, 20.47 mmol) and potassium acetate (5 g, 50.95 mmol). The mixture was degassed with nitrogen for 10 min and PdCl₂(dppf)·DCM (1.4 g, 1.71 mmol) was added and the resulting mixture was degassed under nitrogen for an additional 10 min. The tube was sealed and heated at 120° C. for 2 h. The mixture was cooled to room temperature, diluted with ethyl acetate (50 mL) and filtered through Celite®. A saturated solution of ammonium chloride (25 mL) was added, and the layers were separated. The aqueous layer was extracted with ethyl acetate (3×50 mL). The combined organic layer was washed with brine, dried over anhydrous magnesium sulfate, filtered and concentrated in vacuo. Purification by silica gel chromatography using heptanes provided 4,4,5,5-tetramethyl-2-[2-methyl-4-(2,2,2-trifluoro-1,1-dimethyl-ethyl)phenyl]-1,3,2-dioxaborolane (Intermediate B-13, 4.42 g, 79%) as a white powder. ESI-MS m/z calc. 328.18, found 329.2 (M+1)⁺. ¹H NMR (400 MHz, DMSO-d₆) δ 7.63 (d, J=7.8 Hz, 1H), 7.35-7.29 (m, 2H), 2.48 (s, 3H), 1.53 (s, 6H), 1.29 (s, 12H) ppm. ¹⁹F NMR (377 MHz, DMSO-d₆) δ −74.75 (s, 3F) ppm.

### Intermediate B-14

### 2-[4-(1,1-dimethylpropyl)-2-methyl-phenyl]-4,4,5,5-tetramethyl-1,3,2-dioxaborolane

### Step 1: 2-[4-(1,1-dimethylpropyl)-2-methyl-phenyl]-4,4,5,5-tetramethyl-1,3,2-dioxaborolane (Intermediate B-14)

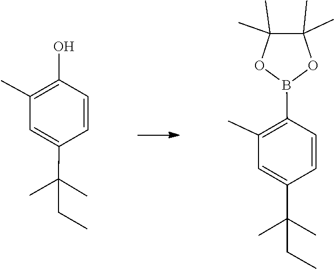

2-[4-(1,1-dimethylpropyl)-2-methyl-phenyl]-4,4,5,5-tetramethyl-1,3,2-dioxaborolane (Intermediate B-14) was prepared from 2-methyl-4-(tert-pentyl)phenol using a procedure analogous to that found in Intermediate B-13 (Step 4 and Step 5) by using microwave irradiation at 120° C. for 1 h. NMR (400 MHz, DMSO-d₆) δ 7.64-7.45 (m, 1H), 7.11 (d, J=7.5 Hz, 2H), 2.45 (s, 3H), 1.60 (q, J=7.4 Hz, 2H), 1.28 (s, 12H), 1.21 (s, 6H), 0.60 (t, J=7.4 Hz, 3H) ppm.

### Intermediate B-15

### 2-[4-tert-butyl-2-methyl-5-(trifluoromethyl)phenyl]-4,4,5,5-tetramethyl-1,3,2-dioxaborolane

### Step 1: 5-bromo-4-tert-butyl-2-methyl-phenol

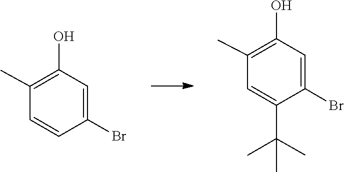

To a solution of 5-bromo-2-methyl-phenol (1 g, 5.35 mmol) and 2-methylpropan-2-ol (1.6 mL, 16.73 mmol) in heptane (5 mL) cooled to 0° C. was added sulfuric acid (570 μL, 10.69 mmol) and the mixture was stirred at room temperature for 16 h. The mixture was quenched with water and ethyl acetate was added. The layers were separated, and the aqueous layer was extracted with ethyl acetate (2×). The combined organic layer was dried over magnesium sulfate, filtered and concentrated. Purification via silica gel column chromatography using 0 to 5% ethyl acetate in hexanes gave 5-bromo-4-tert-butyl-2-methyl-phenol (760 mg, 58%). ESI-MS m/z calc. 242.03, found 243.0 (M+1)⁺. ¹H NMR (400 MHz, DMSO-d₆) δ 9.51 (s, 1H), 7.14 (s, 1H), 7.01 (s, 1H), 2.07 (s, 3H), 1.41 (s, 9H) ppm.

### Step 2: 1-benzyloxy-5-bromo-4-tert-butyl-2-methyl-benzene

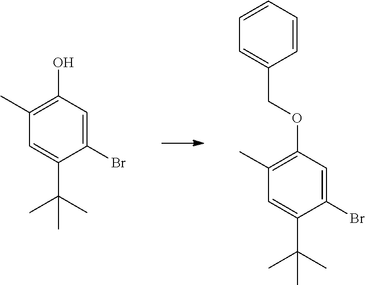 [SMILES: Cc1cc(C(C)(C)C)c(Br)cc1O ; Cc1cc(C(C)(C)C)c(Br)cc1OCc1ccccc1]

A solution of 5-bromo-4-tert-butyl-2-methyl-phenol (658 mg, 2.706 mmol) benzyl bromide (355 μL, 2.99 mmol) and potassium carbonate (450 mg, 3.26 mmol) in acetonitrile (7 mL) was heated at reflux for 16 h. The mixture was quenched with water and the aqueous layer was extracted with DCM (3×). The combined organic layer was dried over magnesium sulfate, filtered and concentrated. Purification via silica gel column chromatography using hexanes gave 1-benzyloxy-5-bromo-4-tert-butyl-2-methyl-benzene (750 mg, 83%). ¹H NMR (400 MHz, DMSO-d₆) δ 7.48-7.37 (m, 4H), 7.36-7.30 (m, 1H), 7.25 (d, J=0.9 Hz, 1H), 7.20 (s, 1H), 5.12 (s, 2H), 2.15 (s, 3H), 1.43 (s, 9H) ppm.

### Step 3: (5-benzyloxy-2-tert-butyl-4-methyl-phenyl)boronic acid

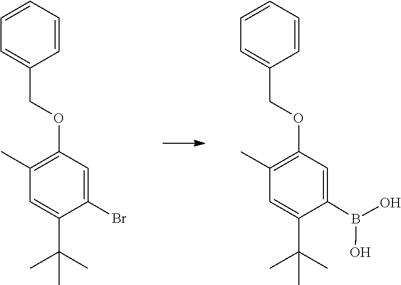 [SMILES: Cc1cc(C(C)(C)C)c(Br)cc1OCc1ccccc1 ; Cc1cc(C(C)(C)C)c(B(O)O)cc1OCc1ccccc1]

A tube was charged with 1-benzyloxy-5-bromo-4-tert-butyl-2-methyl-benzene (100 mg, 0.27 mmol), XPhos Pd G4 (20 mg, 0.02 mmol), XPhos (22 mg, 0.05 mmol), hypoboric acid (102 mg, 1.14 mmol), potassium acetate (115 mg, 1.17 mmol) and ethanol (2 mL). The solution was bubbled with nitrogen for 5 min, sealed and stirred at 85° C. for 18 h. The crude was partitioned between water (20 mL) and ethyl acetate (20 mL). The aqueous phase was extracted with ethyl acetate (2×20 mL). The combined organic layer was dried over anhydrous sodium sulfate, filtered and concentrated under reduced pressure. The crude product was purified by reversed-phase chromatography (C₁₈, 2-60% acetonitrile/water, containing 0.1% formic acid) to afford (5-benzyloxy-2-tert-butyl-4-methyl-phenyl)boronic acid (62 mg, 74%) as a white solid. ¹H NMR (400 MHz, CD₃OD) δ 7.51-7.43 (m, 2H), 7.42-7.35 (m, 2H), 7.34-7.27 (m, 1H), 7.21 (s, 1H), 6.84 (s, 1H), 5.09 (s, 2H), 2.23 (s, 3H), 1.39 (s, 9H) ppm.

### Step 4: 1-benzyloxy-4-tert-butyl-2-methyl-5-(trifluoromethyl)benzene

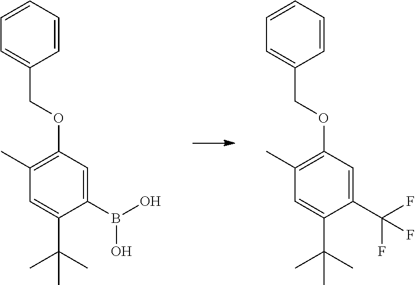 [SMILES: Cc1cc(C(C)(C)C)c(B(O)O)cc1OCc1ccccc1 ; Cc1cc(C(C)(C)C)c(C(F)(F)F)cc1OCc1ccccc1]

To a solution of (5-benzyloxy-2-tert-butyl-4-methyl-phenyl)boronic acid (1.58 g, 5.29 mmol), copper(I) chloride (580 mg, 5.86 mmol) and sodium trifluoromethanesulfonate (2.7 g, 17.3 mmol) in a mixture of methanol (21 mL), DCM (21 mL) and water (17 mL) at room temperature was slowly added tert-butyl hydroperoxide in water (4.3 mL of 70% w/v, 33.4 mmol) and the reaction was stirred at room temperature for 18 h. Additional tert-butyl hydroperoxide in water (2 mL of 70% w/v, 15.54 mmol), copper(I) chloride (270 mg, 2.73 mmol) and sodium trifluoromethanesulfonate (1.7 g, 10.89 mmol) were added and the reaction was stirred for an additional 18 h. The mixture was diluted with water (50 mL) and extracted with DCM (2×50 mL). The combined organic layer was dried over sodium sulfate, filtered and concentrated under reduced pressure. The crude product was purified by silica gel chromatography using heptanes to afford 1-benzyloxy-4-tert-butyl-2-methyl-5-(trifluoromethyl)benzene (1.01 g, 58%) as a colorless oil. ¹H NMR (400 MHz, CDCl₃) δ 7.51-7.31 (m, 6H), 7.23 (s, 1H), 5.09 (s, 2H), 2.30 (s, 3H), 1.45 (s, 9H) ppm. ¹⁹F NMR (377 MHz, CDCl₃) δ −52.61 (s, 3F) ppm.

### Step 5: 4-tert-butyl-2-methyl-5-(trifluoromethyl)phenol

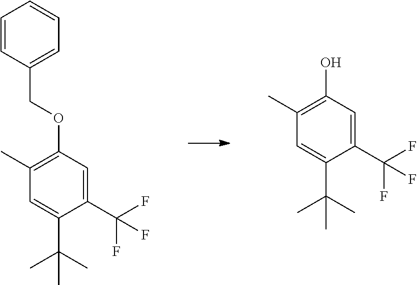 [SMILES: Cc1cc(C(C)(C)C)c(C(F)(F)F)cc1OCc1ccccc1 ; Cc1cc(C(C)(C)C)c(C(F)(F)F)cc1O]

A solution of 1-benzyloxy-4-tert-butyl-2-methyl-5-(trifluoromethyl)benzene (210 mg, 0.65 mmol) in methanol (5 mL) was treated with 10% Pd/C (69 mg, 0.065 mmol) and sparged with hydrogen for 1 h. The mixture was filtered and concentrated to give 4-tert-butyl-2-methyl-5-(trifluoromethyl)phenol (150 mg, 99%). ¹H NMR (400 MHz, CDCl₃) δ 7.36 (s, 1H), 7.12 (s, 1H), 4.87 (s, 1H), 2.27 (s, 3H), 1.41 (s, 9H) ppm. ¹⁹F NMR (376 MHz, CDCl₃) δ −52.94 ppm.

### Step 6: [4-tert-butyl-2-methyl-5-(trifluoromethyl)phenyl]trifluoromethanesulfonate

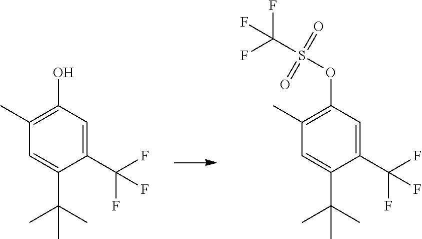 [SMILES: Cc1cc(C(C)(C)C)c(C(F)(F)F)cc1O ; Cc1cc(C(C)(C)C)c(C(F)(F)F)cc1OS(=O)(=O)C(F)(F)F]

A mixture of 4-tert-butyl-2-methyl-5-(trifluoromethyl)phenol (500 mg, 2.15 mmol) and pyridine (530 μL, 6.55 mmol) in DCM (15 mL) was carefully treated with trifluoromethylsulfonyl trifluoromethanesulfonate (550 μL, 3.27 mmol) at 0° C. The mixture was stirred overnight at room temperature and quenched with water and DCM. The organic layer was separated, dried over sodium sulfate, filtered and evaporated in vacuo. The obtained material was purified by silica gel chromatography using 0 to 5% ethyl acetate in hexanes to give [4-tert-butyl-2-methyl-5-(trifluoromethyl)phenyl]trifluoromethanesulfonate (732 mg, 93%). ¹H NMR (400 MHz, CDCl₃) δ 7.57 (s, 1H), 7.55 (s, 1H), 2.42 (s, 3H), 1.45 (s, 9H) ppm. ¹⁹F NMR (376 MHz, CDCl₃) δ −53.38, −73.62 ppm.

### Step 7: 2-[4-tert-butyl-2-methyl-5-(trifluoromethyl)phenyl]-4,4,5,5-tetramethyl-1,3,2-dioxaborolane (Intermediate B-15)

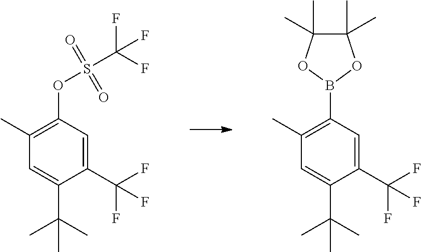 [SMILES: Cc1cc(C(C)(C)C)c(C(F)(F)F)cc1OS(=O)(=O)C(F)(F)F ; Cc1cc(C(C)(C)C)c(C(F)(F)F)cc1B1OC(C)(C)C(C)(C)O1]

A microwave reaction vial charged with [4-tert-butyl-2-methyl-5-(trifluoromethyl)phenyl]trifluoromethanesulfonate (730 mg, 2.004 mmol), Pd(dppf)Cl₂·DCM (82 mg, 0.1 mmol), Et₃N (850 μL, 6.1 mmol) and 1,4-dioxane (10 mL) was purged with nitrogen and pinacolborane (870 μL, 6 mmol) was added under nitrogen atmosphere and the vial was sealed. The mixture was subjected to microwave irradiation at 140° C. for 1 h. The mixture was quenched with water and extracted with ethyl acetate. The organic layer was washed with brine, dried over sodium sulfate, filtered and concentrate. The residue was purified by silica gel column chromatography using hexanes to give 2-[4-tert-butyl-2-methyl-5-(trifluoromethyl)phenyl]-4,4,5,5-tetramethyl-1,3,2-dioxaborolane (Intermediate B-15, 500 mg, 69%) as a yellow oil. ESI-MS m/z calc. 342.19, found 343.3 (M+1)⁺. ¹H NMR (400 MHz, CDCl₃) δ 8.09 (s, 1H), 7.41 (s, 1H), 2.56 (s, 3H), 1.43 (s, 9H), 1.33 (s, 12H) ppm. ¹⁹F NMR (376 MHz, CDCl₃) δ −52.36 ppm.

### Intermediate B-16

### 2-(4-tert-butyl-2-fluoro-3,6-dimethyl-phenyl)-4,4,5,5-tetramethyl-1,3,2-dioxaborolane

### Step 1: 2-bromo-4-tert-butyl-3,6-dimethyl-phenol

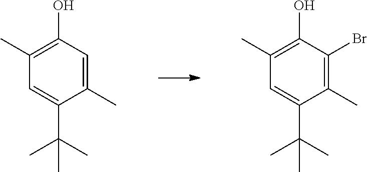

To a solution of 4-tert-butyl-2,5-dimethyl-phenol (10.1 g, 48.16 mmol) in acetonitrile (100 mL) was added NBS (11 g, 61.80 mmol). The mixture was stirred at room temperature for 17 h. Additional NBS (8 g, 44.95 mmol) was added. The mixture was stirred at rt for 6 h. The mixture was poured onto a stirring mixture of saturated sodium bicarbonate (100 mL), sodium thiosulfate (100 mL) and MTBE (200 mL). The layers were separated and the organic layer was washed with brine (50 mL), dried over sodium sulfate, filtered and evaporated under reduced pressure. The residue was triturated with heptanes (30 mL). The solid was removed by filtration and washed with heptanes. The filtrate was concentrated under reduced pressure. The residue was purified by silica gel column chromatography using 0 to 10% ethyl acetate in heptanes to give 2-bromo-4-tert-butyl-3,6-dimethyl-phenol (12.3 g, 99%) as a light-yellow solid. GCMS m/z calc. 256.04, found 255.80 (M). ¹H NMR (400 MHz, CDCl₃) δ 7.07-7.04 (m, 1H), 2.42 (s, 3H), 1.99 (d, J=1.3 Hz, 3H), 1.15 (s, 9H) ppm.

### Step 2: 3-bromo-1-tert-butyl-4-(methoxymethoxy)-2,5-dimethyl-benzene

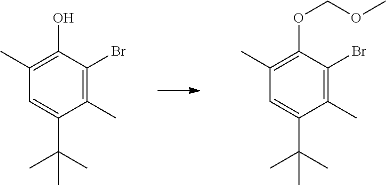

To a solution of 2-bromo-4-tert-butyl-3,6-dimethyl-phenol (66 mg, 0.26 mmol) and DIPEA (103.88 mg, 0.14 mL, 0.80 mmol) in DCM (2 mL) was added chloromethyl methyl ether (53 mg, 0.05 mL, 0.66 mmol). The mixture was stirred at rt for 21 h and diluted with DCM (80 mL). The organic layer was washed with saturated aqueous sodium bicarbonate (20 mL), dried over anhydrous sodium sulfate, filtered and evaporated under reduced pressure to give 3-bromo-1-tert-butyl-4-(methoxymethoxy)-2,5-dimethyl-benzene (70 mg, 80%) as a brown oil. ¹H NMR (400 MHz, CDCl₃) δ 7.13 (s, 1H), 5.04 (s, 2H), 3.66 (s, 3H), 2.56 (s, 3H), 2.33 (s, 3H), 1.40 (s, 9H) ppm.

### Step 3: 1-tert-butyl-3-fluoro-4-(methoxymethoxy)-2,5-dimethyl-benzene

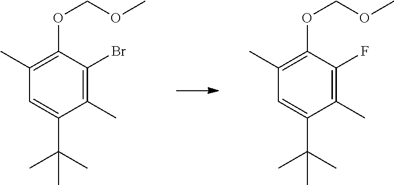

To a solution of 3-bromo-1-tert-butyl-4-(methoxymethoxy)-2,5-dimethyl-benzene (1.09 g, 3.61 mmol) in THF (20 mL) at −78° C. was slowly added a solution of n-BuLi in hexanes (1.7 mL of 2.5 M, 4.25 mmol) under nitrogen atmosphere. The mixture was then stirred at this temperature for 30 min after which, a solution of N-fluorobenzenesulfonimide (1.3 g, 4.12 mmol) in THF (20 mL) was added dropwise over 20 min. The resulting mixture was stirred for 1 h at −78° C. The mixture was warmed to room temperature, diluted with water (20 mL) and extracted with ethyl acetate (2×40 mL). The combined organic layers were washed with water (40 mL), dried over anhydrous sodium sulfate, filtered and concentrated under reduced pressure. The crude product was purified by silica gel column chromatography using 0 to 20% ethyl acetate in heptanes to afford 1-tert-butyl-3-fluoro-4-(methoxymethoxy)-2,5-dimethyl-benzene (827 mg, 72%) as a clear oil. ¹H NMR (400 MHz, CDCl₃) δ 6.93 (s, 1H), 5.10 (s, 2H), 3.62 (s, 3H), 2.39 (d, J=3.6 Hz, 3H), 2.29 (s, 3H), 1.40 (s, 9H) ppm. ¹⁹F NMR (376 MHz, CDCl₃) δ −131.65 (s, 1F) ppm.

### Step 4: 4-tert-butyl-2-fluoro-3,6-dimethyl-phenol

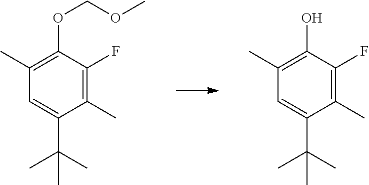

Hydrochloric acid (3 mL of 6 M, 18 mmol) was added to a solution of 1-tert-butyl-3-fluoro-4-(methoxymethoxy)-2,5-dimethyl-benzene (827 mg, 2.62 mmol) in tetrahydrofuran (10 mL) and mixture was stirred at room temperature for 18 h. The mixture was diluted with water (50 mL) and extracted using MTBE (3×50 mL). The organic layers were combined, washed with brine (50 mL), dried over sodium sulfate and concentrated under reduced pressure. The crude product was purified by silica gel chromatography using 0 to 20% ethyl acetate in heptanes to afford 4-tert-butyl-2-fluoro-3,6-dimethyl-phenol (508 mg, 99%) as a clear oil. ¹H NMR (400 MHz, CDCl₃) δ 6.89 (s, 1H), 4.95 (d, J=5.7 Hz, 1H), 2.40 (d, J=3.4 Hz, 3H), 2.25 (s, 3H), 1.39 (s, 9H) ppm. ¹⁹F NMR (376 MHz, CDCl₃) δ −142.65 (s, 1F) ppm.

### Step 5: 2-(4-tert-butyl-2-fluoro-3,6-dimethyl-phenyl)-4,4,5,5-tetramethyl-1,3,2-dioxaborolane (Intermediate B-16)

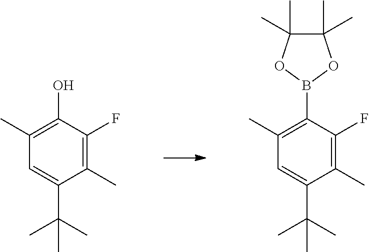 [SMILES: Cc1cc(C(C)(C)C)c(C)c(F)c1O ; Cc1cc(C(C)(C)C)c(C)c(F)c1B1OC(C)(C)C(C)(C)O1]

2-(4-tert-butyl-2-fluoro-3,6-dimethyl-phenyl)-4,4,5,5-tetramethyl-1,3,2-dioxaborolane (Intermediate B-16) was prepared from 4-tert-butyl-2-fluoro-3,6-dimethyl-phenol using procedure analogous to that found in Intermediate B-12. ESI-MS m/z calc. 306.22, found 307.3 (M+1)⁺. ¹H NMR (400 MHz, CDCl₃) δ 6.96 (s, 1H), 2.42 (s, 3H), 2.36 (d, J=3.4 Hz, 3H), 1.43-1.38 (m, 21H) ppm.

### Intermediate B-17

### 2-[4-tert-butyl-2-(2-methoxyethoxy)phenyl]-4,4,5,5-tetramethyl-1,3,2-dioxaborolane

### Step 1: 1-bromo-4-tert-butyl-2-(2-methoxyethoxy)benzene

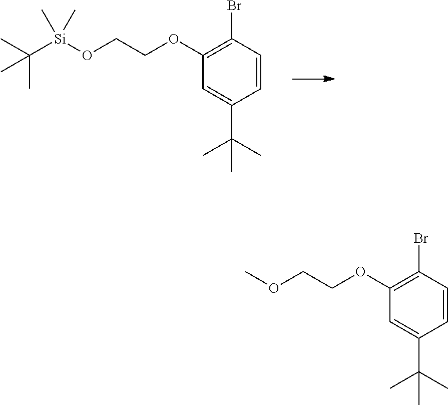 [SMILES: CC(C)(C)c1ccc(Br)c(OCCO[Si](C)(C)C(C)(C)C)c1 ; COCCOc1cc(C(C)(C)C)ccc1Br]

A mixture of 2-(2-bromo-5-tert-butyl-phenoxy)ethoxy-tert-butyl-dimethyl-silane (93 mg, 0.24 mmol) in DCM (4 mL) was treated with a solution of tetrabutylammonium fluoride (1 mL of 1 M in THF, 1 mmol) and stirred in a sealed vial at room temperature for 2 h. The mixture was diluted with water (˜10 mL) and extracted with DCM (2×30 mL). The combined organic extracts were dried over sodium sulfate, filtered and concentrated in vacuo to provide 2-(2-bromo-5-tert-butyl-phenoxy)ethanol. The 2-(2-bromo-5-tert-butyl-phenoxy)ethanol was dissolved in THF (4 mL) and the solution cooled to 0° C. NaH (50 mg, 60% w/w, 1.3 mmol) was added and the mixture stirred at 0° C. for 15 min. Iodomethane (50 L, 0.80 mmol) was added dropwise and the mixture stirred at 0° C. for 30 min, then warmed to room temperature and stirred 1.5 h. The mixture was quenched with saturated aqueous ammonium chloride (˜10 mL) and extracted with DCM (3×20 mL). The combined organic extracts were dried over sodium sulfate, filtered and concentrated in vacuo. Purification by silica gel chromatography (0-30% ethyl acetate/hexanes) afforded 1-bromo-4-tert-butyl-2-(2-methoxyethoxy)benzene (45 mg, 65%). ¹H NMR (400 MHz, DMSO-d₆) δ 7.45 (d, J=8.3 Hz, 1H), 7.08 (d, J=2.2 Hz, 1H), 6.90 (dd, J=8.3, 2.2 Hz, 1H), 4.23-4.15 (m, 2H), 3.72-3.64 (m, 2H), 3.35 (s, 3H), 1.27 (s, 9H) ppm.

### Step 2: 2-[4-tert-butyl-2-(2-methoxyethoxy)phenyl]-4,4,5,5-tetramethyl-1,3,2-dioxaborolane (Intermediate B-17)

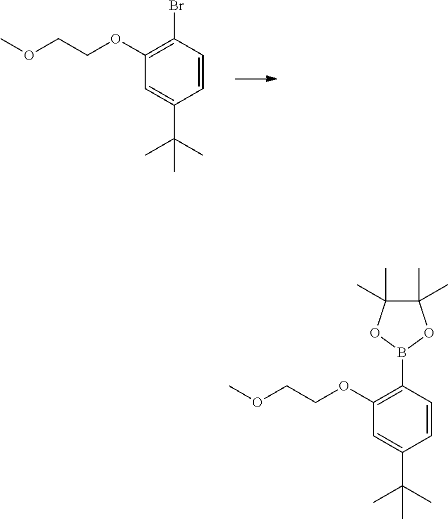

A vial containing a mixture of 1-bromo-4-tert-butyl-2-(2-methoxyethoxy)benzene (45 mg, 0.16 mmol), 4,4,5,5-tetramethyl-2-(4,4,5,5-tetramethyl-1,3,2-dioxaborolan-2-yl)-1,3,2-dioxaborolane (85 mg, 0.34 mmol), potassium acetate (90 mg, 0.92 mmol) and Pd(dppf)Cl₂·DCM (34 mg, 0.042 mmol) in 1,4-dioxane (5 mL) was flushed with nitrogen for 30 seconds, capped and stirred at 85° C. for 2.5 h. The mixture was cooled, diluted with ethyl acetate, and washed with saturated aqueous saturated sodium bicarbonate and brine. The organic layer dried over sodium sulfate, filtered and concentrated in vacuo to provide 2-[4-tert-butyl-2-(2-methoxyethoxy)phenyl]-4,4,5,5-tetramethyl-1,3,2-dioxaborolane (Intermediate B-17). ESI-MS m/z calc. 334.2, found 335.4 (M+1)⁺.

### Example 3

### General Procedure for Suzuki Coupling of 2-chloro-6-methylpyridin-4-ol with Intermediate B

### Method—A

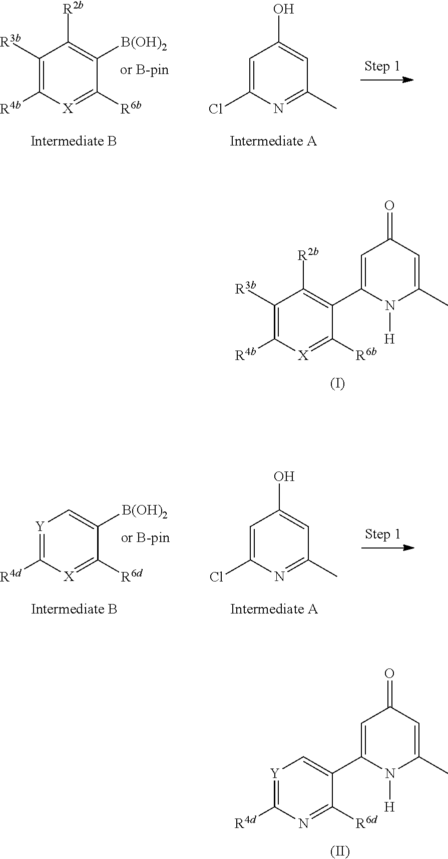

A microwave vial charged with Intermediate A (1 eq), Intermediate B (1-2 eq, custom or commercial boronic acid or boronic ester), XPhos Pd G3 (1-5 mol %), X-Phos (1-10 mol %,), potassium carbonate (2-3 eq), ethanol and water was degassed under an atmosphere of nitrogen for 1-2 minutes. The vial was sealed and subjected to microwave irradiation at 100 to 120° C. for 30 minutes or heated thermally at 60 to 100° C. for 16 hours. The mixture was filtered and purified via reverse phase HPLC to obtain the desired products.

The following compounds (Table 2) were synthesized via Method A using commercially available boronic acid or boronic esters.

TABLE 2

| | | LC/MS | |
|---|---|---|---|
| | | (m/z calc.); | |
| Cmpd | | Found | |
| No. | Chemical Name | (m + H) | NMR (shifts in ppm) |
| 1 | 2-indan-5-yl-6-methyl-1H-pyridin- | 225.29 | |
| | 4-one | 226.17 | |
| 2 | 2-(4-tert-butyl-2-methyl-phenyl)-6- | 255.35 | |
| | methyl-1H-pyridin-4-one | 256.3 | |
| 3 | 2-(4-tert-butyl-2,6-dimethyl- | 269.38 | ¹H NMR (500 MHz, CD₃OD) δ 7.30 (s, |
| | phenyl)-6-methyl-1H-pyridin-4-one | 270.28 | 2H), 7.11 (d, J = 2.4 Hz, 1H), 6.98 (d, J = |
| | | | 2.4 Hz, 1H), 2.65 (s, 3H), 2.16 (s, 6H), |
| | | | 1.36 (s, 9H). |
| 4 | 2-[2-hydroxy-4- | 269.22 | |
| | (trifluoromethyl)phenyl]-6-methyl- | 269.98 | |
| | 1H-pyridin-4-one | | |
| 5 | 2-[2-methoxy-4- | 283.25 | |
| | (trifluoromethyl)phenyl]-6-methyl- | 284.33 | |
| | 1H-pyridin-4-one | | |
| 6 | 2-methyl-6-[3-methyl-4- | 283.25 | |
| | (trifluoromethoxy)phenyl]-1H- | 284.38 | |
| | pyridin-4-one | | |
| 7 | 2-methyl-6-[3-methyl-5- | 267.25 | ¹H NMR (400 MHz, DMSO-d₆) δ 13.98 |
| | (trifluoromethyl)phenyl]-1H- | 268.33 | (s, 1H), 8.04 (s, 1H), 8.02 (s, 1H), 7.77 (s, |
| | pyridin-4-one | | 1H), 7.39 (s, 1H), 7.10 (s, 1H), 2.63 (s, |
| | | | 3H), 2.51 (s, 3H). |
| 8 | 2-(4-methoxy-2,5-dimethyl- | 243.3 | ¹H NMR (400 MHz, DMSO-d₆) δ 13.98 |
| | phenyl)-6-methyl-1H-pyridin-4-one | 244.42 | (s, 1H), 7.23 (s, 1H), 7.10 (s, 1H), 7.07 (s, |
| | | | 1H), 6.99 (s, 1H), 3.86 (s, 3H), 2.59 (s, |
| | | | 3H), 2.27 (s, 3H), 2.17 (s, 3H). |
| 9 | 2-(2-tert-butylphenyl)-6-methyl- | 241.33 | ¹H NMR (400 MHz, DMSO-d₆) δ 14.32 |
| | 1H-pyridin-4-one | 242.42 | (s, 1H), 7.71-7.64 (m, 1H), 7.55 (t, 1H), |
| | | | 7.36 (t, 1H), 7.22 (d, 1H), 7.18 (s, 2H), |
| | | | 2.58 (s, 3H), 1.18 (s, 9H). |
| 10 | 2-(5-methoxy-2-methyl-phenyl)-6- | 229.27 | |
| | methyl-1H-pyridin-4-one | 230.1 | |
| 11 | 2-(3-fluoro-2-methyl-phenyl)-6- | 217.24 | |
| | methyl-1H-pyridin-4-one | 218.02 | |
| 12 | 2-(3-methoxy-5-methyl-phenyl)-6- | 229.27 | ¹H NMR (400 MHz, DMSO-d₆) δ 13.35 |
| | methyl-1H-pyridin-4-one | 230.1 | (s, 1H), 7.35 (d, J = 2.4 Hz, 1H), 7.25 (d, |
| | | | J = 2.9 Hz, 2H), 7.12 (d, J = 2.3 Hz, 1H), |
| | | | 7.05-6.99 (m, 1H), 3.85 (s, 3H), 2.65 (s, |
| | | | 3H), 2.39 (s, 3H). |
| 13 | 2-(3-tert-butyl-5-methyl-phenyl)-6- | 255.35 | ¹H NMR (400 MHz, DMSO-d₆) δ 13.86 |
| | methyl-1H-pyridin-4-one | 256.47 | (s, 1H), 7.61 (s, 1H), 7.48 (s, 2H), 7.35 (d, |
| | | | 1H), 7.13 (d, 1H), 2.67 (s, 3H), 2.42 (s, |
| | | | 3H), 1.35 (s, 9H). |
| 14 | 2-(2-methoxyphenyl)-6-methyl-1H- | 215.25 | |
| | pyridin-4-one | 216.02 | |
| 15 | 2-(4-tert-butoxyphenyl)-6-methyl- | 257.33 | |
| | 1H-pyridin-4-one | 258.07 | |
| 16 | 2-└2-(hydroxymethyl)-5- | 283.25 | |
| | (trifluoromethyl)phenyl]-6-methyl- | 283.98 | |
| | 1H-pyridin-4-one | | |
| 17 | 2-(2,2-difluoro-1,3-benzodioxol-4- | 265.21 | |
| | yl)-6-methyl-1H-pyridin-4-one | 265.93 | |
| 18 | 2-(3-ethoxy-2-fluoro-5-methyl- | 261.29 | |
| | phenyl)-6-methyl-1H-pyridin-4-one | 262.43 | |
| 19 | 2-[2-ethoxy-4- | 297.27 | |
| | (trifluoromethyl)phenyl]-6-methyl- | 298.29 | |
| | 1H-pyridin-4-one | | |
| 20 | 2-methyl-6-(1-methylindol-5-yl)- | 238.28 | |
| | 1H-pyridin-4-one | 239 | |
| 21 | 2-methyl-6-(o-tolyl)-1H-pyridin-4- | 199.25 | |
| | one | 200.43 | |
| 22 | 2-(2-methoxy-6-methyl-phenyl)-6- | 229.27 | |
| | methyl-1H-pyridin-4-one | 230.18 | |
| 23 | 2-(2,4-dimethylphenyl)-6-methyl- | 213.28 | |
| | 1H-pyridin-4-one | 214 | |
| 24 | 2-methyl-6-[3-methyl-4- | 267.25 | ¹H NMR (400 MHz, DMSO-d₆) δ 13.42 |
| | (trifluoromethyl)phenyl]-1H- | 268.33 | (s, 1H), 7.99 (s, 1H), 7.87 (q, 2H), 7.34 (s, |
| | pyridin-4-one | | 1H), 7.05 (s, 1H), 2.62 (s, 3H), 2.55 (s, |
| | | | 3H). |
| 25 | 2-[2-methoxy-5- | 283.25 | |
| | (trifluoromethyl)phenyl]-6-methyl- | 284.28 | |
| | 1H-pyridin-4-one | | |
| 26 | 2-(2,3-difluorophenyl)-6-methyl- | 221.2 | |
| | 1H-pyridin-4-one | 221.97 | |
| 27 | 2-(4-isopropoxy-3,5-dimethyl- | 271.35 | |
| | phenyl)-6-methyl-1H-pyridin-4-one | 272.43 | |
| 28 | 2-(4-methoxy-2-methyl-phenyl)-6- | 229.27 | |
| | methyl-1H-pyridin-4-one | 230.37 | |
| 29 | 2-(4-methoxy-3,5-dimethyl- | 243.3 | |
| | phenyl)-6-methyl-1H-pyridin-4-one | 244.07 | |
| 30 | 2-methyl-6-[2-methyl-4- | 283.25 | |
| | (trifluoromethoxy)phenyl]-1H- | 284.33 | |
| | pyridin-4-one | | |
| 31 | 2-(2,6-dimethylphenyl)-6-methyl- | 213.27 | |
| | 1H-pyridin-4-one | 214.07 | |
| 32 | 2-[4-methoxy-3- | 283.25 | |
| | (trifluoromethyl)phenyl]-6-methyl- | 284.28 | |
| | 1H-pyridin-4-one | | |
| 33 | 2-methyl-6-[4- | 269.22 | |
| | (trifluoromethoxy)phenyl]-1H- | 270.03 | |
| | pyridin-4-one | | |
| 34 | 2-(4-cyclopropyl-2-methyl-phenyl)- | 239.31 | ¹H NMR (400 MHz, DMSO-d₆) δ 14.02 |
| | 6-methyl-1H-pyridin-4-one | 240.42 | (s, 1H), 7.31 (d, 1H), 7.20 (s, 2H), 7.13 (s, |
| | | | 1H), 7.08 (d, 1H), 2.62 (s, 3H), 2.25 (s, |
| | | | 3H), 2.03-1.92 (m, 1H), 1.05-0.95 (m, |
| | | | 2H), 0.78-0.67 (m, 2H). |
| 35 | 2-(3,4-dimethylphenyl)-6-methyl- | 213.28 | |
| | 1H-pyridin-4-one | 214.07 | |
| 36 | 2-[4-fluoro-3- | 271.21 | ¹H NMR (400 MHz, DMSO-d₆) δ 13.57 |
| | (trifluoromethyl)phenyl]-6-methyl- | 272.28 | (s, 1H), 8.33 (d, 1H), 8.29-8.20 (m, 1H), |
| | 1H-pyridin-4-one | | 7.75 (t, 1H), 7.42 (d, J = 2.4 Hz, 1H), 7.15 |
| | | | (d, J = 2.3 Hz, 1H), 2.65 (s, 3H). |
| 37 | 2-(2,3-dimethylphenyl)-6-methyl- | 213.28 | |
| | 1H-pyridin-4-one | 214.07 | |
| 38 | 2-methyl-6-[3- | 269.22 | |
| | (trifluoromethoxy)phenyl]-1H- | 270.33 | |
| | pyridin-4-one | | |
| 39 | 2-methyl-6-[2- | 269.22 | |
| | (trifluoromethoxy)phenyl┘-1H- | 269.98 | |
| | pyridin-4-one | | |
| 40 | 2-(2,3-dimethoxy-5-methyl- | 259.3 | ¹H NMR (400 MHz, DMSO-d₆) δ 13.88 |
| | phenyl)-6-methyl-1H-pyridin-4-one | 260.13 | (s, 1H), 7.27 (d, J = 2.4 Hz, 1H), 7.18 (d, |
| | | | J = 2.4 Hz, 1H), 7.15 (s, 1H), 6.92 (s, 1H), |
| | | | 3.88 (s, 3H), 3.66 (s, 3H), 2.62 (s, 3H), |
| | | | 2.35 (s, 3H). |
| 41 | 2-methyl-6-tetralin-6-yl-1H- | 239.31 | ¹H NMR (400 MHz, DMSO-d₆) δ 13.15 |
| | pyridin-4-one | 240.42 | (s, 1H), 7.58 (s, 1H), 7.56 (d, 1H), 7.24 (d, |
| | | | J = 7.9 Hz, 1H), 7.15 (d, J = 2.3 Hz, 1H), |
| | | | 6.89 (s, 1H), 2.85-2.75 (m, 4H), 2.56 (s, |
| | | | 3H), 1.83-1.72 (m, J = 3.8, 3.1 Hz, 4H). |
| 42 | 2-indan-4-yl-6-methyl-1H-pyridin- | 225.29 | ¹H NMR (400 MHz, DMSO-d₆) δ 13.75 |
| | 4-one | 226.07 | (s, 1H), 7.47 (d, 1H), 7.42-7.31 (m, 2H), |
| | | | 7.17 (d, J = 2.4 Hz, 1H), 7.08 (d, J = 2.4 |
| | | | Hz, 1H), 2.96 (q, J = 7.7 Hz, 4H), 2.60 (s, |
| | | | 3H), 2.04 (p, J = 7.4 Hz, 2H). |
| 43 | 2-(2,4-diisopropylphenyl)-6- | 269.38 | ¹H NMR (400 MHz, DMSO-d₆) δ 13.86 |
| | methyl-1H-pyridin-4-one | 270.5 | (s, 1H), 7.39 (d, J = 1.6 Hz, 1H), 7.32- |
| | | | 7.20 (m, 2H), 7.09-6.97 (m, 2H), 2.99 |
| | | | (hept, J = 6.9 Hz, 1H), 2.84-2.73 (m, |
| | | | 1H), 2.55 (s, 3H), 1.25 (d, J = 6.9 Hz, 6H), |
| | | | 1.17 (d, J = 6.8 Hz, 6H). |
| 44 | 2-(2-methoxy-3-methyl-phenyl)-6- | 229.27 | |
| | methyl-1H-pyridin-4-one | 230.07 | |
| 45 | 2-(5-acetyl-2-methoxy-phenyl)-6- | 257.28 | ¹H NMR (400 MHz, DMSO-d₆) δ 14.05 |
| | methyl-1H-pyridin-4-one | 257.97 | (s, 1H), 8.23-8.17 (m, 1H), 8.12 (d, J = |
| | | | 2.2 Hz, 1H), 7.37 (d, 1H), 7.24 (d, J = 2.4 |
| | | | Hz, 1H), 7.11 (d, J = 2.4 Hz, 1H), 3.93 (s, |
| | | | 3H), 2.62 (s, 3H), 2.59 (s, 3H). |
| 46 | 2-methyl-6-[2-methyl-5- | 267.25 | |
| | (trifluoromethyl)phenyl]-1H- | 268.38 | |
| | pyridin-4-one | | |
| 47 | 2-(4-cyclopropyl-2-fluoro-phenyl)- | 243.28 | ¹H NMR (400 MHz, DMSO-d₆) δ 13.74 |
| | 6-methyl-1H-pyridin-4-one | 244.02 | (s, 1H), 7.62 (t, 1H), 7.24 (s, 1H), 7.18 (s, |
| | | | 1H), 7.17-7.09 (m, 2H), 2.61 (s, 3H), |
| | | | 2.12-2.02 (m, 1H), 1.13-1.02 (m, 2H), |
| | | | 0.87-0.78 (m, 2H). |
| 48 | 2-(5-tert-butyl-2-methyl-phenyl)-6- | 255.35 | |
| | methyl-1H-pyridin-4-one | 256.47 | |
| 49 | 2-[2-isopropoxy-5- | 312.29 | ¹H NMR (400 MHz, DMSO-d₆) δ 13.76 |
| | (trifluoromethyl)-3-pyridyl]-6- | 313.56 | (s, 1H), 8.76 (s, 1H), 8.45 (d, J = 2.4 Hz, |
| | methyl-1H-pyridin-4-one | | 1H), 7.39 (d, J = 2.4 Hz, 1H), 7.07 (s, 1H), |
| | | | 5.44 (hept, J = 6.1 Hz, 1H), 2.60 (s, 3H), |
| | | | 1.35 (d, J = 5.9 Hz, 6H). |
| 50 | 2-methyl-6-[6-methyl-5- | 268.23 | |
| | (trifluoromethyl)-3-pyridyl]-1H- | 269.51 | |
| | pyridin-4-one | | |
| 51 | 2-methyl-6-[5-methyl-6- | 268.23 | |
| | (trifluoromethyl)-3-pyridyl]-1H- | 269.51 | |
| | pyridin-4-one | | |
| 52 | 2-[2-ethoxy-5-(trifluoromethyl)-3- | 298.26 | ¹H NMR (400 MHz, DMSO-d₆) δ 13.57 |
| | pyridyl]-6-methyl-1H-pyridin-4- | 299.53 | (s, 1H), 8.77 (s, 1H), 8.45 (d, J = 2.5 Hz, |
| | one | | 1H), 7.43 (d, J = 2.4 Hz, 1H), 7.16 (d, J = |
| | | | 2.4 Hz, 1H), 4.49 (q, 2H), 2.63 (s, 3H), |
| | | | 1.34 (t, J = 7.0 Hz, 3H). |
| 53 | 2-(2,5-dimethoxy-4-methyl- | 259.3 | ¹H NMR (400 MHz, DMSO-d₆) δ 13.82 |
| | phenyl)-6-methyl-1H-pyridin-4-one | 260.54 | (s, 1H), 7.26 (d, J = 2.5 Hz, 1H), 7.13 (s, |
| | | | 1H), 7.12 (d, J = 2.4 Hz, 1H), 7.11 (s, 1H), |
| | | | 3.82 (s, 3H), 3.79 (s, 3H), 2.63 (s, 3H), |
| | | | 2.25 (s, 3H). |
| 54 | 2-(2-methoxy-4,6-dimethyl- | 243.3 | |
| | phenyl)-6-methyl-1H-pyridin-4-one | 244.59 | |
| 55 | 2-(4-methoxy-2,3-dimethyl- | 243.3 | |
| | phenyl)-6-methyl-1H-pyridin-4-one | 244.59 | |
| 56 | 2-(2-fluoro-4,5-dimethyl-phenyl)-6- | 231.27 | |
| | methyl-1H-pyridin-4-one | 232 | |
| 57 | 2-(2-methoxy-4,5-dimethyl- | 243.3 | ¹H NMR (400 MHz, DMSO-d₆) δ 13.70 |
| | phenyl)-6-methyl-1H-pyridin-4-one | 244.59 | (s, 1H), 7.30 (s, 1H), 7.20 (d, J = 2.4 Hz, |
| | | | 1H), 7.08 (dd, J = 2.4, 0.7 Hz, 1H), 7.07 |
| | | | (s, 1H), 3.81 (s, 3H), 2.60 (s, 3H), 2.32 (s, |
| | | | 3H), 2.23 (s, 3H). |
| 58 | 2-methyl-6-[5-(trifluoromethyl)-3- | 254.21 | |
| | pyridyl]-1H-pyridin-4-one | 255.48 | |
| 59 | 2-[3-fluoro-4- | 271.21 | |
| | (trifluoromethyl)phenyl]-6-methyl- | 272.2 | |
| | 1H-pyridin-4-one | | |
| 60 | 2-(2,5-dimethylphenyl)-6-methyl- | 213.27 | |
| | 1H-pyridin-4-one | 214 | |
| 61 | 2-[2-fluoro-4- | 271.21 | |
| | (trifluoromethyl)phenyl]-6-methyl- | 272.2 | |
| | 1H-pyridin-4-one | | |
| 62 | 2-[2-methoxy-3- | 283.25 | ¹H NMR (400 MHz, DMSO-d₆) δ 14.30 |
| | (trifluoromethyl)phenyl]-6-methyl- | 284.3 | (s, 1H), 7.92 (d, J = 1.6 Hz, 1H), 7.90 (d, |
| | 1H-pyridin-4-one | | J = 1.6 Hz, 1H), 7.53 (t, 1H), 7.36 (d, J = |
| | | | 2.4 Hz, 1H), 7.24 (d, J = 2.4 Hz, 1H), 3.52 |
| | | | (s, 3H), 2.65 (s, 3H). |
| 63 | 2-[2-methoxy-5-(trifluoromethyl)- | 284.23 | |
| | 3-pyridyl]-6-methyl-1H-pyridin-4- | 284 | |
| | one | | |
| 64 | 2-(2-chloro-5-fluoro-4-methyl- | 251.68 | |
| | phenyl)-6-methyl-1H-pyridin-4-one | 252.1 | |
| 65 | 2-(3-tert-butoxyphenyl)-6-methyl- | 257.33 | |
| | 1H-pyridin-4-one | 258.2 | |
| 66 | 2-(2-hydroxyphenyl)-6-methyl-1H- | 201.22 | ¹H NMR (400 MHz, DMSO-d₆) δ 13.84 |
| | pyridin-4-one | 202.2 | (s, 1H), 7.51 (dd, J = 7.7, 1.7 Hz, 1H), |
| | | | 7.44-7.37 (m, 1H), 7.28 (d, J = 2.4 Hz, |
| | | | 1H), 7.12-7.04 (m, 2H), 6.99 (td, J = 7.5, |
| | | | 1.1 Hz, 1H), 2.59 (s, 3H). |
| 67 | 2-(2-fluoro-3-methyl-phenyl)-6- | 217.24 | ¹H NMR (400 MHz, DMSO-d₆) δ 13.70 |
| | methyl-1H-pyridin-4-one | 218.2 | (s, 1H), 7.58-7.48 (m, 2H), 7.31 (t, J = |
| | | | 7.7 Hz, 1H), 7.12 (s, 1H), 7.00 (s, 1H), |
| | | | 2.57 (s, 3H), 2.33 (s, 3H). |
| 68 | 2-[3-fluoro-5- | 271.21 | |
| | (trifluoromethyl)phenyl]-6-methyl- | 272.1 | |
| | 1H-pyridin-4-one | | |
| 69 | 2-(3,5-dimethylphenyl)-6-methyl- | 213.27 | ¹H NMR (400 MHz, DMSO-d₆) δ 13.68 |
| | 1H-pyridin-4-one | 214.2 | (s, 1H), 7.47 (s, 2H), 7.27 (s, 2H), 7.06 (s, |
| | | | 1H), 2.62 (d, J = 3.0 Hz, 3H), 2.38 (d, J = |
| | | | 3.0 Hz, 6H). |
| 70 | 2-(2-methoxy-4-methyl-phenyl)-6- | 229.27 | ¹H NMR (400 MHz, DMSO-d₆) δ 13.78 |
| | methyl-1H-pyridin-4-one | 230.2 | (s, 1H), 7.41 (d, J = 7.7 Hz, 1H), 7.20 (d, |
| | | | J = 2.4 Hz, 1H), 7.10 (t, J = 2.3 Hz, 2H), |
| | | | 6.97 (dd, J = 7.8, 1.1 Hz, 1H), 3.83 (s, |
| | | | 3H), 2.60 (s, 3H), 2.41 (s, 3H). |
| 71 | 2-(3-fluoro-4-methyl-phenyl)-6- | 217.24 | ¹H NMR (400 MHz, DMSO-d₆) δ 13.80 |
| | methyl-1H-pyridin-4-one | 218.2 | (s, 1H), 7.79-7.70 (m, 1H), 7.67-7.58 |
| | | | (m, 1H), 7.52 (t, J = 7.9 Hz, 1H), 7.35 (d, |
| | | | J = 2.2 Hz, 1H), 7.10 (s, 1H), 2.64 (s, 3H), |
| | | | 2.33 (s, 3H). |
| 72 | 2-(6-chloroindan-5-yl)-6-methyl- | 259.73 | |
| | 1H-pyridin-4-one | 260.2 | |
| 73 | 2-(3-chloro-2,4-dimethyl-phenyl)- | 247.72 | ¹H NMR (400 MHz, DMSO-d₆) δ 14.16 |
| | 6-methyl-1H-pyridin-4-one | 248.2 | (s, 1H), 7.42 (d, J = 7.9 Hz, 1H), 7.34 (d, |
| | | | J = 7.8 Hz, 1H), 7.14 (s, 1H), 7.09 (s, 1H), |
| | | | 2.59 (s, 3H), 2.44 (s, 3H), 2.27 (s, 3H). |
| 74 | 2-(4-chloro-2-methyl-phenyl)-6- | 233.69 | |
| | methyl-1H-pyridin-4-one | 234.1 | |

### Example 4

### General Procedure for Suzuki Coupling of Benzyl-Protected Intermediate A with Intermediate B

### Method C

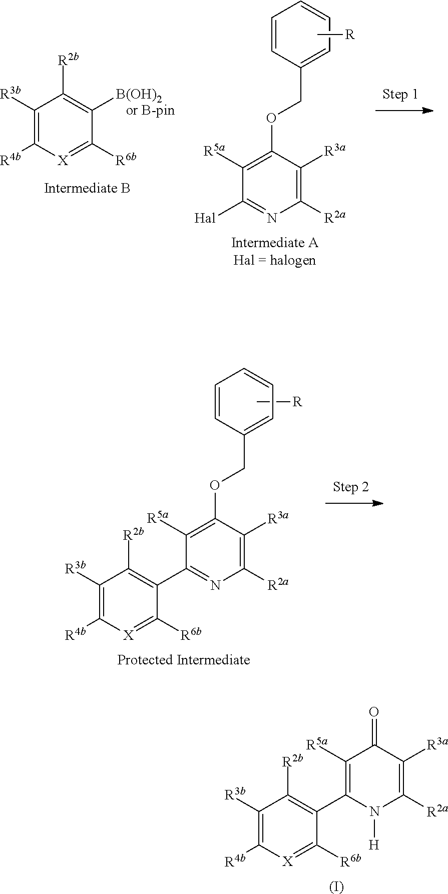

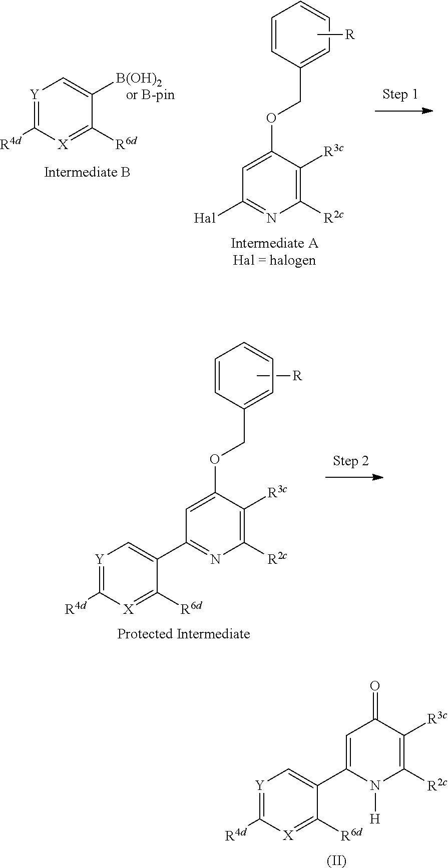

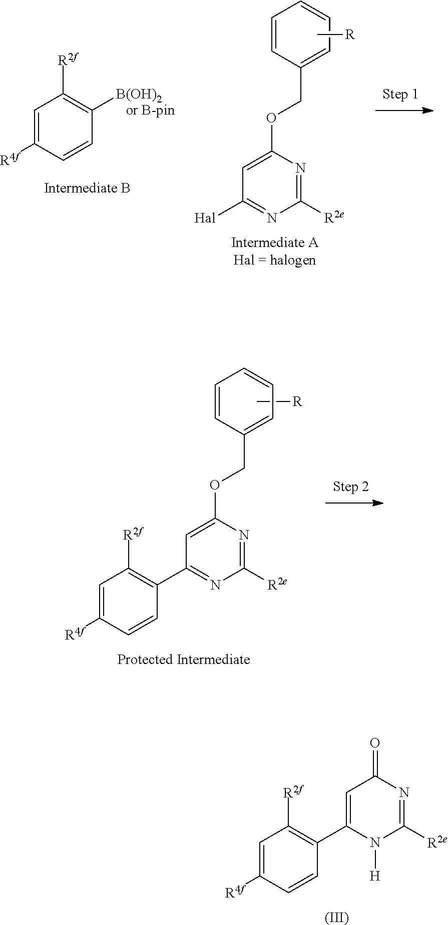

Step 1: A mixture of Intermediate A (1 eq), Intermediate B (1-2 eq, custom or commercial boronic acid or boronic ester), Palladium source (1-5 mol %, e.g. PdCl₂(dppf) or PdCl₂(dtbpf), base (2-3 eq, eg. potassium phosphate) in organic solvent (e.g. 1,4-dioxane, DMSO, toluene) and water is degassed with nitrogen bubbling nitrogen and stirred under nitrogen atmosphere at a temperature ranging from room temperature to 120° C. The mixture is filtered and purified via silica gel column chromatography or reverse phase HPLC to obtain the indicated protected intermediate.
Step 2: A mixture of the protected intermediate and Pd/C is stirred in the appropriate solvent (e.g. methanol, ethanol, or ethyl acetate) under an atmosphere of hydrogen. The mixture is filtered, concentrated, and purified via silica gel column chromatography or reverse phase column chromatography to provide the desired product of formula (I), (II), or (III) as outlined in Table 3.

Alternatively, a solution of the protected intermediate in the appropriate solvent (DCM, 1,4-dioxane or toluene) is treated with acid (e.g. HCl or TFA) and stirred at room temperature or 60-70° C. The mixture is subsequently neutralized and purified via silica gel column chromatography or reverse phase column chromatography to provide the desired product of formula (I), (II), or (III) as outlined in Table 3.

TABLE 3

| | | LC/MS | |
|---|---|---|---|
| | | (m/z calc.); | |
| Cmpd | | Found | |
| No. | Chemical Name | (m + H) | NMR (shifts in ppm) |
| 75 | 2-(4-tert-butyl-2-methyl- | 269.38 | ¹H NMR (500 MHz, DMSO-d₆) δ 11.05 |
| | phenyl)-3,6-dimethyl-1H- | 270.3 | (s, 1H), 7.39 (d, J = 2.1 Hz, 1H), 7.34 (dd, |
| | pyridin-4-one | | J = 8.0, 2.1 Hz, 1H), 7.16 (d, J = 8.0 Hz, |
| | | | 1H), 5.91 (s, 1H), 2.16 (s, 3H), 2.13 (s, |
| | | | 3H), 1.55 (s, 3H), 1.32 (s, 9H). |
| 76 | 6-(4-tert-butyl-2-methyl- | 269.38 | ¹H NMR (500 MHz, DMSO-d₆) δ 7.45 (d, |
| | phenyl)-2,3-dimethyl-1H- | 270.4 | J = 2.1 Hz, 1H), 7.41 (dd, J = 8.0, 2.0 Hz, |
| | pyridin-4-one | | 1H), 7.34 (d, J = 8.0 Hz, 1H), 7.20 (s, 1H), |
| | | | 2.57 (s, 3H), 2.27 (s, 3H), 2.13 (s, 3H), |
| | | | 1.33 (s, 9H). |
| 77 | 2-(4-tert-butylphenyl)-3,6- | 255.35 | |
| | dimethyl-1H-pyridin-4-one | 256.3 | |
| 78 | 3,6-dimethyl-2-[4-[1-(trifluoro- | 307.31 | |
| | methyl)cyclopropyl]phenyl]- | 308.3 | |
| | 1H-pyridin-4-one | | |
| 79 | 6-(4-tert-butylphenyl)-2,3- | 255.35 | ¹H NMR (500 MHz, DMSO-d₆) δ 13.85 |
| | dimethyl-1H-pyridin-4-one | 256.32 | (s, 1H), 7.80-7.72 (m, 2H), 7.69-7.61 |
| | | | (m, 2H), 7.48 (s, 1H), 2.67 (s, 3H), 2.16 |
| | | | (s, 3H), 1.35 (s, 9H). |
| 80 | 2,3-dimethyl-6-(o-tolyl)-1H- | 213.27 | |
| | pyridin-4-one | 214.3 | |
| 81 | 6-(4-tert-butyl-2-methoxy- | 285.38 | |
| | phenyl)-2,3-dimethyl-1H- | 286.3 | |
| | pyridin-4-one | | |
| 82 | 2,3-dimethyl-6-[4-[1-(trifluoro- | 307.31 | ¹H NMR (500 MHz, DMSO-d₆) δ 14.02 |
| | methyl)cyclopropyl]phenyl]- | 308.46 | (s, 1H), 7.88-7.81 (m, 2H), 7.72 (d, J = |
| | 1H-pyridin-4-one | | 8.0 Hz, 2H), 7.58 (s, 1H), 2.69 (s, 3H), |
| | | | 2.17 (s, 3H), 1.42 (t, J = 3.5 Hz, 2H), |
| | | | 1.27-1.19 (m, 2H). |
| 83 | 4-(4-tert-butyl-2-methyl- | 256.34 | |
| | phenyl)-2-methyl-1H- | 257.5 | |
| | pyrimidin-6-one | | |
| 84 | 6-(2-methoxyphenyl)-2,3- | 229.27 | ¹H NMR (500 MHz, DMSO-d₆) δ 14.20 |
| | dimethyl-1H-pyridin-4-one | 230.23 | (s, 1H), 7.52 (td, J = 7.2, 2.0 Hz, 1H), |
| | | | 7.47-7.39 (m, 3H), 7.37 (s, 1H), 2.63 (s, 3H), |
| | | | 2.27 (s, 3H), 2.18 (s, 3H). |
| 85 | 2,3-dimethyl-6-[4-(2,2,2- | 309.33 | |
| | trifluoro-1,1-dimethyl- | 310.33 | |
| | ethyl)phenyl]-1H-pyridin-4- | | |
| | one | | |
| 86 | ethyl 6-(4-tert-butyl-2-methyl- | 327.42 | |
| | phenyl)-2-methyl-4-oxo-1H- | 328.36 | |
| | pyridine-3-carboxylate | | |
| 87 | 2,3-dimethyl-6-[2-methyl-4- | 321.34 | ¹H NMR (400 MHz, DMSO-d₆) δ 11.14 |
| | [1-(trifluoromethyl)cyclo- | 322.55 | (s, 1H), 7.45 (s, 1H), 7.40 (d, J = 7.8 Hz, |
| | propyl]phenyl]-1H-pyridin- | | 1H), 7.31 (d, J = 7.7 Hz, 1H), 5.87 (s, 1H), |
| | 4-one | | 2.26 (s, 3H), 2.23 (s, 3H), 1.86 (s, 3H), |
| | | | 1.40-1.32 (m, 2H), 1.15 (s, 2H). |
| 88 | 6-[4-(3,3-difluorocyclobutyl)- | 303.35 | ¹H NMR (400 MHz, DMSO-d₆) δ 11.09 |
| | 2-methyl-phenyl]-2,3- | 304.28 | (s, 1H), 7.28 (d, J = 7.9 Hz, 1H), 7.25 (d, |
| | dimethyl-1H-pyridin-4-one | | J = 2.4 Hz, 2H), 5.88-5.83 (m, 1H), 3.44 |
| | | | (p, J = 8.6 Hz, 1H), 3.10-2.94 (m, 2H), |
| | | | 2.76-2.63 (m, 2H), 2.25 (s, 3H), 2.23 (s, |
| | | | 3H), 1.86 (s, 3H). |
| 89 | 2,3-dimethyl-6-[2-methyl-4- | 294.35 | |
| | (3-methylisoxazol-5- | 295.4 | |
| | yl)phenyl]-1H-pyridin-4-one | | |
| 90 | 6-[2-methoxy-4-(3- | 310.35 | |
| | methylisoxazol-5-yl)phenyl]- | 311.4 | |
| | 2,3-dimethyl-1H-pyridin-4- | | |
| | one | | |
| 91 | 6-[4-(1,1-dimethylpropyl)-2- | 283.41 | ¹H NMR (400 MHz, CD₃OD) δ 7.35- |
| | methyl-phenyl]-2,3-dimethyl- | 284.2 | 7.27 (m, 2H), 7.24 (d, J = 8.0 Hz, 1H), |
| | 1H-pyridin-4-one | | 6.29 (s, 1H), 2.38 (s, 3H), 2.29 (s, 3H), |
| | | | 2.07 (s, 3H), 1.70 (q, J = 7.4 Hz, 2H), 1.31 |
| | | | (s, 6H), 0.69 (t, J = 7.4 Hz, 3H). |
| 92 | 6-[4-(3,3-difluoro-1-methyl- | 317.37 | ¹H NMR (400 MHz, DMSO-d₆) δ δ 11.09 |
| | cyclobutyl)-2-methyl-phenyl]- | 318.5 | (s, 1H), 7.28-7.10 (m, 3H), 5.87 (d, J = |
| | 2,3-dimethyl-1H-pyridin-4- | | 1.6 Hz, 1H), 3.02-2.88 (m, 2H), 2.85- |
| | one | | 2.71 (m, 2H), 2.26 (s, 3H), 2.23 (s, 2H), |
| | | | 1.86 (s, 3H), 1.48 (s, 3H). |
| 93 | 2,3-dimethyl-6-[2-methyl-4- | 323.35 | ¹H NMR (400 MHz, CD₃OD) δ 7.54- |
| | (2,2,2-trifluoro-1,1-dimethyl- | 324.02 | 7.46 (m, 2H), 7.33 (d, J = 8.1 Hz, 1H), |
| | ethyl)phenyl]-1H-pyridin-4- | | 6.29 (s, 1H), 2.38 (s, 3H), 2.31 (s, 3H), |
| | one | | 2.07 (s, 3H), 1.65-1.53 (m, 6H). |
| 94 | ethyl 2-(4-tert-butyl-2-methyl- | 341.44 | ¹H NMR (400 MHz, CD₃OD) δ 7.37 (d, J = |
| | phenyl)-5,6-dimethyl-4-oxo- | 342.3 | 2.0 Hz, 1H), 7.34-7.25 (m, 1H), 7.16 |
| | 1H-pyridine-3-carboxylate | | (d, J = 8.1 Hz, 1H), 4.0-3.90 (m, 2H), |
| | | | 2.39-2.27 (m, 3H), 2.23 (s, 3H), 2.12- |
| | | | 2.03 (m, 3H), 1.33 (s, 9H), 0.81 (t, J = 7.1 |
| | | | Hz, 3H). |
| 95 | 6-(2-methoxy-3-quinolyl)-2,3- | 280.32 | ¹H NMR (400 MHz, DMSO-d₆) δ 8.60 (s, |
| | dimethyl-1H-pyridin-4-one | 281.4 | 1H), 8.02 (dd, J = 8.1, 1.4 Hz, 1H), 7.89 |
| | | | (d, J = 8.9 Hz, 1H), 7.82 (ddd, J = 8.4, |
| | | | 6.8, 1.5 Hz, 1H), 7.56 (ddd, J = 8.1, 6.8, |
| | | | 1.3 Hz, 1H), 7.26 (s, 1H), 4.05 (s, 3H), |
| | | | 2.61 (s, 3H), 2.18 (s, 3H). N—H proton is |
| | | | missing/overlapped with a solvent. |
| 96 | 2-methyl-6-[2-methyl-4-[1- | 307.31 | |
| | (trifluoromethyl)cyclopro- | 308.4 | |
| | pyl]phenyl]-1H-pyridin-4-one | | |
| 97 | ethyl 5,6-dimethyl-2-[2- | 393.4 | |
| | methyl-4-[1-(trifluoro- | 394.6 | |
| | methyl)cyclopropyl]phenyl]- | | |
| | 4-oxo-1H-pyridine-3-carboxylate | | |
| 98 | ethyl 2-(4-tert-butyl-2,6- | 355.47 | ¹H NMR (400 MHz, DMSO-d₆) δ 11.35 |
| | dimethyl-phenyl)-5,6- | 356.98 | (s, 1H), 7.14 (s, 2H), 3.81 (q, J = 7.1 Hz, |
| | dimethyl-4-oxo-1H-pyridine- | | 2H), 2.24 (s, 3H), 2.10 (s, 6H), 1.88 (s, |
| | 3-carboxylate | | 3H), 1.27 (s, 9H), 0.65 (t, J = 7.1 Hz, 3H). |
| 99 | ethyl 2-(4-tert-butyl-2- | 357.44 | |
| | methoxy-phenyl)-5,6- | 358.7 | |
| | dimethyl-4-oxo-1H-pyridine- | | |
| | 3-carboxylate | | |
| 100 | 2-(4-tert-butyl-2-methyl- | 284.4 | ¹H NMR (400 MHz, DMSO-d₆) δ 12.46 |
| | phenyl)-6-(dimethylamino)- | 285.6 | (s, 1H), 12.00 (s, 1H), 7.43-7.31 (m, 3H), |
| | 1H-pyridin-4-one | | 6.43 (s, 1H), 6.31 (s, 1H), 3.14 (s, 6H), |
| | (Hydrochloride salt) | | 2.29 (s, 3H), 1.32 (s, 9H). |
| 101 | 2-methyl-6-[2- | 255.2 | ¹H NMR (400 MHz, CD₃OD) δ 9.39 (s, |
| | (trifluoromethyl)pyrimidin-5- | 256.1 | 2H), 7.46-7.37 (m, 1H), 7.18-7.09 (m, |
| | yl]-1H-pyridin-4-one | | 1H), 2.71 (d, J = 7.1 Hz, 3H). |
| 102 | 6-(4-tert-butyl-2-methyl- | 285.38 | ¹H NMR (400 MHz, DMSO-d₆) δ 14.03 |
| | phenyl)-3-methoxy-2-methyl- | 286.6 | (s, 1H), 7.48-7.39 (m, 2H), 7.34 (d, J = |
| | 1H-pyridin-4-one | | 8.0 Hz, 1H), 7.17 (d, J = 4.1 Hz, 1H), 3.88 |
| | | | (s, 3H), 2.52 (s, 3H), 2.27 (s, 3H), 1.32 (s, |
| | | | 9H). |
| 103 | 2-(2-tert-butylpyrimidin-5-yl)- | 243.3 | ¹H NMR (400 MHz, CD₃OD) δ 9.12 (s, |
| | 6-methyl-1H-pyridin-4-one | 244.2 | 2H), 7.38 (d, J = 2.4 Hz, 1H), 7.14 (d, J = |
| | | | 2.4 Hz, 1H), 2.72 (s, 3H), 1.46 (s, 9H). ¹H |
| | | | NMR (400 MHz, DMSO-d₆) δ 14.40 (s, |
| | | | 1H), 9.21 (s, 2H), 7.46 (d, J = 2.4 Hz, 1H), |
| | | | 7.19 (d, J = 2.3 Hz, 1H), 2.66 (s, 3H), 1.42 |
| | | | (s, 9H). |
| 104 | 2-(4-tert-butyl-2-methyl- | 271.35 | ¹H NMR (400 MHz, DMSO-d₆) δ 7.36- |
| | phenyl)-6-methoxy-1H- | 272.5 | 7.29 (m, 3H), 6.71 (s, 1H), 6.45 (s, 1H), |
| | pyridin-4-one | | 3.93 (s, 3H), 2.31 (s, 3H), 1.31 (s, 9H). |
| 105 | 2-(4-tert-butyl-2-methyl- | 309.33 | |
| | phenyl)-6-(trifluoromethyl)- | 310.3 | |
| | 1H-pyridin-4-one | | |
| 106 | 2-(4-tert-butyl-2-methyl- | 299.41 | ¹H NMR (400 MHz, DMSO-d₆) δ 13.97 |
| | phenyl)-3-ethoxy-6-methyl- | 300.5 | (s, 1H), 7.45 (d, J = 1.9 Hz, 1H), 7.41 (dd, |
| | 1H-pyridin-4-one | | J = 8.1, 2.0 Hz, 1H), 7.34 (d, J = 8.1 Hz, |
| | | | 1H), 7.23 (s, 1H), 2.52 (s, 3H), 2.51-2.50 |
| | | | (m, 2H), 2.18 (s, 3H), 1.33 (s, 9H), 0.95 (t, |
| | | | J = 7.0 Hz, 3H). |
| 107 | 2-(4-tert-butyl-2-methyl- | 285.38 | |
| | phenyl)-3-methoxy-6-methyl- | 286.1 | |
| | 1H-pyridin-4-one | | |
| 108 | 6-(4-tert-butyl-2-methyl- | 289.8 | |
| | phenyl)-3-chloro-2-methyl- | 290.3 | |
| | 1H-pyridin-4-one | | |
| 109 | ethyl 6-(4-tert-butyl-2,5- | 341.44 | |
| | dimethyl-phenyl)-2-methyl-4- | 342.6 | |
| | oxo-1H-pyridine-3- | | |
| | carboxylate | | |
| 110 | 6-(4-tert-butyl-2-methyl- | 298.42 | ¹H NMR (400 MHz, DMSO-d₆) δ 14.06- |
| | phenyl)-3-(dimethylamino)-2- | 299.4 | 13.61 (br s, 1H), 7.45 (d, J = 1.9 Hz, 1H), |
| | methyl-1H-pyridin-4-one | | 7.41 (dd, J = 8.0, 2.0 Hz, 1H), 7.34 (d, J = |
| | | | 8.0 Hz, 1H), 7.08 (s, 1H), 2.85 (s, 6H), |
| | | | 2.58 (s, 3H), 2.27 (s, 3H), 1.32 (s, 9H) |
| 111 | ethyl 2-(4-tert-butyl-2,5- | 366.45 | |
| | dimethyl-phenyl)-5-cyano-6- | 367.5 | |
| | methyl-4-oxo-1H-pyridinc-3- | | |
| | carboxylate | | |
| 112 | 3-chloro-2-methyl-6-[2- | 343.77 | ¹H NMR (400 MHz, DMSO-d₆) δ 12.28 |
| | methyl-4-(2,2,2-trifluoro-1,1- | 344.4 | (s, 1H), 7.55 (s, 1H), 7.51 (d, J = 8.1 Hz, |
| | dimethyl-ethyl)phenyl]-1H- | | 1H), 7.38 (d, J = 8.1 Hz, 1H), 6.36 (s, 1H), |
| | pyridin-4-one | | 2.44 (s, 3H), 2.30 (s, 3H), 1.59 (s, 6H). |
| 113 | 6-(4-tert-butyl-2,5-dimethyl- | 283.41 | ¹H NMR (400 MHz, DMSO-d₆) δ 13.81 |
| | phenyl)-2,3-dimethyl-1H- | 284.3 | (s, 1H), 7.36 (s, 1H), 7.18 (s, 1H), 7.13 (s, |
| | pyridin-4-one | | 1H), 2.58 (s, 3H), 2.54 (s, 3H), 2.23 (s, |
| | | | 3H), 2.16 (s, 3H), 1.42 (s, 9H). |
| 114 | 3-amino-6-(4-tert-butyl-2- | 270.37 | ¹H NMR (400 MHz, DMSO-d₆) δ 13.80 |
| | methyl-phenyl)-2-methyl-1H- | 271.2 | (s, 1H), 7.43 (d, J = 2.0 Hz, 1H), 7.39 (dd, |
| | pyridin-4-onc | | J = 8.1, 2.0 Hz, 1H), 7.30 (d, J = 8.0 Hz, |
| | | | 1H), 7.08 (d, J = 1.6 Hz, 1H), 2.47 (s, 3H), |
| | | | 2.24 (s, 3H), 1.32 (s, 9H). |
| 115 | 3-chloro-6-[2,5-dimethyl-4- | 357.8 | ¹H NMR (400 MHz, DMSO-d₆) δ 7.50 (s, |
| | (2,2,2-trifluoro-1,1-dimethyl- | 358.3 | 1H), 7.21 (s, 1H), 6.39 (s, 1H), 2.52 (s, |
| | ethyl)phenyl]-2-methyl-1H- | | 3H), 2.45 (s, 3H), 2.25 (s, 3H), 1.70 (s, |
| | pyridin-4-one | | 6H). N—H proton is missing. |
| 116 | 2-(4-tert-butyl-2,5-dimethyl- | 269.38 | ¹H NMR (400 MHz, DMSO-d₆) δ 7.32 (s, |
| | phenyl)-6-methyl-1H-pyridin- | 270.5 | 1H), 7.16 (s, 1H), 6.76 (s, 1H), 6.74 (s, |
| | 4-one | | 1H), 2.51 (s, 3H), 2.46 (s, 3H), 2.23 (s, |
| | | | 3H), 1.39 (s, 9H). |
| 117 | 6-[2,5-dimethyl-4-(2,2,2- | 337.38 | ¹H NMR (400 MHz, DMSO-d₆) δ 7.47 (s, |
| | trifluoro-1,1-dimethyl- | 338.2 | 1H), 7.19 (s, 1H), 6.38 (s, 1H), 2.53 (s, |
| | ethyl)phenyl]-2,3-dimethyl- | | 3H), 2.37 (s, 3H), 2.25 (s, 3H), 1.99 (s, |
| | 1H-pyridin-4-one | | 3H), 1.71 (s, 6H). |
| 118 | 6-[5-fluoro-2-methyl-4-(2,2,2- | 341.34 | ¹H NMR (400 MHz, DMSO-d₆) δ 14.00 |
| | trifluoro-1,1-dimethyl- | 342.2 | (s, 1H), 7.60 (d, J = 8.1 Hz, 1H), 7.44- |
| | ethyl)phenyl]-2,3-dimethyl- | | 7.36 (m, 1H), 7.10 (s, 1H), 2.58 (s, 3H), |
| | 1H-pyridin-4-one | | 2.27 (s, 3H), 2.17 (s, 3H), 1.69 (s, 6H). |
| 119 | 6-(4-tert-butyl-5-chloro-2- | 366.91 | ¹H NMR (400 MHz, DMSO-d₆) δ 11.68 |
| | methyl-phenyl)-2-methyl-3- | 367.4 | (s, 1H), 7.42 (d, J = 7.9 Hz, 2H), 6.33 (s, |
| | (methylsulfonimidoyl)-1H- | | 1H), 4.20 (s, 1H), 2.68 (s, 3H), 2.29 (s, |
| | pyridin-4-one | | 3H), 1.47 (d, J = 2.6 Hz, 9H). |
| 120 | 6-(4-tert-butyl-5-chloro-2- | 366.91 | ¹H NMR (400 MHz, DMSO-d₆) δ 11.66 |
| | methyl-phenyl)-2-methyl-3- | 367.35 | (s, 1H), 7.54-7.28 (m, 2H), 6.33 (s, 1H), |
| | (methylsulfonimidoyl)-1H- | | 2.66 (s, 3H), 2.29 (s, 3H), 1.47 (s, 9H). |
| | pyridin-4-one | | |
| 121 | 6-(6-tert-butyl-5-chloro-2- | 304.81 | ¹H NMR (400 MHz, DMSO-d₆) δ 14.24 |
| | methyl-3-pyridyl)-2,3- | 305.3 | (s, 1H), 8.02 (s, 1H), 7.31 (s, 1H), 2.62 (s, |
| | dimethyl-1H-pyridin-4-one | | 3H), 2.43 (s, 3H), 2.19 (s, 3H), 1.50 (s, |
| | | | 9H). |
| 122 | 6-(6-tert-butyl-5-chloro-2- | 325.23 | ¹H NMR (400 MHz, DMSO-d₆) δ 7.88 (s, |
| | methyl-3-pyridyl)-3-chloro-2- | 325.2 | 1H), 6.63 (s, 1H), 2.48 (s, 3H), 2.44 (s, |
| | methyl-1H-pyridin-4-one | | 3H), 1.48 (s, 9H). |
| 123 | 6-(6-tert-butyl-2,5-dimethyl-3- | 284.4 | ¹H NMR (400 MHz, DMSO-d₆) δ 14.25 |
| | pyridyl)-2,3-dimethyl-1H- | 285.4 | (s, 1H), 7.62 (s, 1H), 7.34 (s, 1H), 2.62 (s, |
| | pyridin-4-one | | 3H), 2.53 (s, 3H), 2.40 (s, 3H), 2.18 (s, |
| | | | 3H), 1.43 (s, 9H). |
| 124 | 6-(6-tert-butyl-2,5-dimethyl-3- | 304.81 | ¹H NMR (400 MHz, DMSO-d₆) δ 7.61 (s, |
| | pyridyl)-3-chloro-2-methyl- | 305.2 | 1H), 6.80 (s, 1H), 2.54 (s, 3H), 2.52 (s, |
| | 1H-pyridin-4-one | | 3H), 2.43 (s, 3H), 1.43 (s, 9H). |
| 125 | 2-[6-(4-tert-butyl-5-chloro-2- | 346.85 | ¹H NMR (400 MHz, CD₃OD) δ 7.45 (s, |
| | methyl-phenyl)-2-methyl-4- | 347.4 | 1H), 7.35 (s, 1H), 6.30 (s, 1H), 3.54 (s, |
| | oxo-1H-pyridin-3- | | 2H), 2.41 (s, 3H), 2.29 (s, 3H), 1.50 (s, |
| | yl]acetamide | | 9H). |
| 126 | 2-[6-(4-tert-butyl-5-chloro-2- | 360.88 | ¹H NMR (400 MHz, DMSO-d₆) δ 11.31 |
| | methyl-phenyl)-2-methyl-4- | 361.4 | (br s, 1H), 7.69 (br s, 1H), 7.43 (s, 1H), |
| | oxo-1H-pyridin-3-yl]-N- | | 7.35 (s, 1H), 6.01 (s, 1H), 3.26 (s, 2H), |
| | methyl-acetamide | | 2.55 (d, J = 4.5 Hz, 3H), 2.29-2.24 (m, |
| | | | 6H), 1.47 (s, 9H). |
| 127 | 6-(6-tert-butyl-5-chloro-2- | 367.89 | ¹H NMR (400 MHz, CD₃OD) δ 7.75 (s, |
| | methyl-3-pyridyl)-2-methyl-3- | 368.3 | 1H), 6.47 (s, 1H), 3.38 (s, 3H), 2.76 (s, |
| | (methylsulfonimidoyl)-1H- | | 3H), 2.47 (s, 3H), 1.51 (s, 9H). |
| | pyridin-4-one | | |
| 128 | 6-(6-tert-butyl-5-chloro-2- | 367.89 | ¹H NMR (400 MHz, CD₃OD) δ 7.75 (s, |
| | methyl-3-pyridyl)-2-methyl-3- | 368.5 | 1H), 6.47 (s, 1H), 3.38 (s, 3H), 2.76 (s, |
| | (methylsulfonimidoyl)-1H- | | 3H), 2.47 (s, 3H), 1.51 (s, 9H). |
| | pyridin-4-one | | |
| 129 | 6-[5-chloro-2-methyl-4-(2,2,2- | 420.88 | ¹H NMR (400 MHz, DMSO-d₆) δ 12.72 |
| | trifluoro-1,1-dimethyl- | 421.2 | (s, 1H), 9.72 (s, 1H), 7.68 (s, 1H), 7.55 (s, |
| | ethyl)phenyl]-2-methyl-3- | | 1H), 6.53 (s, 1H), 3.93 (s, 3H), 2.70 (s, |
| | (methylsulfonimidoyl)-1H- | | 3H), 2.32 (s, 3H), 1.79 (s, 6H). |
| | pyridin-4-one | | |
| 130 | 6-(4-tert-butyl-2-fluoro-3,6- | 364.48 | ¹H NMR (400 MHz, CD₃OD) δ 7.19 (s, |
| | dimethyl-phenyl)-2-methyl-3- | 365.9 | 1H), 6.38 (s, 1H), 3.38 (s, 3H), 2.74 (s, |
| | (methylsulfonimidoyl)-1H- | | 3H), 2.42 (d, J = 3.5 Hz, 3H), 2.23 (s, 3H), |
| | pyridin-4-one | | 1.44 (s, 9H). |
| 131 | 6-[4-tert-butyl-2-methyl-5- | 400.46 | ¹H NMR (400 MHz, DMSO-d₆) δ 12.72 |
| | (trifluoromethyl)phenyl]-2- | 401.3 | (s, 1H), 9.66 (s, 1H), 7.76 (s, 1H), 7.70 (s, |
| | methyl-3- | | 1H), 6.54 (s, 1H), 3.93 (s, 3H), 2.69 (s, |
| | (methylsulfonimidoyl)-1H- | | 3H), 2.38 (s, 3H), 1.45 (s, 9H). |
| | pyridin-4-one | | |
| 132 | 6-[4-tert-butyl-2-methyl-5- | 400.46 | ¹H NMR (400 MHz, DMSO-d₆) δ 12.30 |
| | (trifluoromethyl)phenyl]-2- | 401.3 | (s, 1H), 9.52 (s, 1H), 7.76 (s, 1H), 7.71 (s, |
| | methyl-3-(methylsulfonimidoyl)- | | 1H), 6.55 (s, 1H), 3.80 (s, 3H), 2.68 (s, |
| | 1H-pyridin-4-one | | 3H), 2.38 (s, 3H), 1.45 (s, 9H). |
| 133 | 6-(4-tert-butyl-5-chloro-2- | 408 13 | ¹H NMR (400 MHz, DMSO-d₆) δ 11.73 |
| | methyl-phenyl)-3-(1,1-dioxo- | 409 | (s, 1H), 7.43 (s, 1H), 7.39 (s, 1H), 6.23 (s, |
| | 1,2-thiazolidin-2-yl)-2-methyl- | | 1H), 3.74 (s, 1H), 3.47 (s, 1H), 3.30 (d, |
| | 1H-pyridin-4-one | | J = 7.3 Hz, 4H), 2.36 (s, 3H), 2.26 (s, 3H), |
| | | | 1.47 (s, 9H). |
| 134 | 6-(4-tert-butyl-5-chloro-2- | 371.4 | ¹H NMR (400 MHz, CD₃OD) δ 7.48 (s, |
| | methyl-phenyl)-2-methyl-3-(5- | 372.4 | 1H), 7.42 (s, 1H), 6.39 (s, 1H), 2.62 (s, |
| | methyl-1,3,4-oxadiazol-2-yl)- | | 3H), 2.42 (s, 3H), 2.33 (s, 3H), 1.51 (s, |
| | 1H-pyridin-4-one | | 9H). |
| 135 | 6-(4-tert-butyl-5-chloro-2- | 371.4 | ¹H NMR (400 MHz, CD₃OD) δ 7.53 (s, |
| | methyl-phenyl)-2-methyl-3-(3- | 372.5 | 1H), 7.51 (s, 1H), 6.89 (br s, 1H), 2.66 (s, |
| | methyl-1,2,4-oxadiazol-5-yl)- | | 3H), 2.51 (s, 3H), 2.35 (s, 3H), 1.52 (s, |
| | 1H-pyridin-4-one | | 9H). |
| 136 | N-[6-(4-tert-butyl-2-methyl- | 348.15 | ¹H NMR (400 MHz, DMSO-d₆) δ 12.17 |
| | phenyl)-2-methyl-4-oxo-1H- | 349 | (s, 1H), 8.61 (s, 1H), 7.39 (d, J = 2.0 Hz, |
| | pyridin-3- | | 1H), 7.36 (dd, J = 8.0, 2.0 Hz, 1H), 7.30 |
| | yl]methanesulfonamide | | (d, J = 7.8 Hz, 1H), 6.40 (s, 1H), 3.05 (s, |
| | | | 3H), 2.40 (s, 3H), 2.28 (s, 3H), 1.32 (s, |
| | | | 9H). |
| 137 | 6-(4-tert-butyl-2,5-dimethyl- | 309.21 | ¹H NMR (400 MHz, DMSO-d₆) δ 13.49 |
| | phenyl)-3-cyclopropyl-2- | 310.3 | (s, 1H), 7.35 (s, 1H), 7.15 (s, 1H), 6.86 (s, |
| | methyl-1H-pyridin-4-one | | 1H), 2.64 (s, 3H), 2.53 (s, 3H), 2.24 (s, |
| | | | 3H), 1.68-1.59 (m, 1H), 1.41 (s, 9H), |
| | | | 1.02-0.96 (m, 2H), 0.83-0.78 (m, 2H). |
| 138 | 6-(4-tert-butyl-5-chloro-2- | 335.11 | ¹H NMR (400 MHz, DMSO-d₆) δ 12.45 |
| | methyl-phenyl)-2-methyl-3- | 336.3 | (br s, 1H), 7.45 (s, 1H), 7.43 (s, 1H), 6.43 |
| | methylsulfanyl-1H-pyridin-4- | | (s, 1H), 2.57 (s, 3H), 2.31 (s, 3H), 2.26 (s, |
| | one | | 3H), 1.47 (s, 9H). |
| 139 | 6-(4-tert-butyl-5-chloro-2- | 394.11 | ¹H NMR (400 MHz, DMSO-d₆) δ 8.54 (s, |
| | methyl-phenyl)-2-methyl-1,1- | 395.1 | 1H), 7.26 (s, 1H), 7.24 (s, 1H), 5.95 (s, |
| | dioxo-4,5-dihydro-3H- | | 1H), 3.54 (t, J = 6.1 Hz, 2H), 2.73 (s, 3H), |
| | pyrido[2,3-e]thiazin-8-one | | 2.70 (t, J = 6.1 Hz, 2H), 2.29 (s, 3H), 1.45 |
| | | | (s, 9H). |
| 140 | 2-(4-tert-butyl-5-chloro-2- | 289.12 | ¹H NMR (400 MHz, CD₃OD) δ 7.53 (s, |
| | methyl-phenyl)-6-methyl-1H- | 290.3 | 1H), 7.49 (s, 1H), 7.06 (d, J = 2.4 Hz, 1H), |
| | pyridin-4-one | | 7.02 (d, J = 2.4 Hz, 1H), 2.64 (s, 3H), 2.30 |
| | | | (s, 3H), 1.52 (s, 9H). |
| 141 | 6-(4-tert-butyl-5-chloro-2- | 303.14 | |
| | methyl-phenyl)-2,3-dimethyl- | 304.3 | |
| | 1H-pyridin-4-one | | |
| 142 | 6-(4-tert-butyl-5-chloro-2- | 314.12 | ¹H NMR (400 MHz, CD₃OD) δ 7.49 (s, |
| | methyl-phenyl)-2-methyl-4- | 315.3 | 1H), 7.42 (s, 1H), 6.52 (s, 1H), 2.64 (s, |
| | oxo-1H-pyridinc-3-carbonitrilc | | 3H), 2.30 (s, 3H), 1.51 (s, 9H). |
| 143 | 3-bromo-6-(4-tert-butyl-5- | 367.03 | ¹H NMR (400 MHz, CD₃OD) δ 7.45 (s, |
| | chloro-2-methyl-phenyl)-2- | 368.19 | 1H), 7.37 (s, 1H), 6.32 (s, 1H), 2.56 (s, |
| | methyl-1H-pyridin-4-one | | 3H), 2.28 (s, 3H), 1.50 (s, 9H). |
| 144 | 6-(4-tert-butyl-5-chloro-2- | 394.15 | ¹H NMR (400 MHz, DMSO-d₆) δ 10.92 |
| | methyl-phenyl)-3- | 395 | (s, 1H), 7.40 (d, J = 3.6 Hz, 2H), 6.46 (s, |
| | (isopropylsulfonimidoyl)-2- | | 1H), 3.70 (p, J = 6.8 Hz, 1H), 2.67 (s, 3H), |
| | methyl-1H-pyridin-4-one | | 2.29 (s, 3H), 1.47 (s, 9H), 1.23 (d, J = 7.0 |
| | | | Hz, 3H), 1.17 (d, J = 6.8 Hz, 3H). |
| 145 | 6-(4-tert-butyl-5-chloro-2- | 394.15 | ¹H NMR (400 MHz, DMSO-d₆) δ 11.10 |
| | methyl-phenyl)-3- | 395 | (s, 1H), 7.40 (d, J = 3.6 Hz, 2H), 6.45 (s, |
| | (isopropylsulfonimidoyl)-2- | | 1H), 3.77-3.62 (m, 1H), 2.68 (s, 3H), |
| | methyl-1H-pyridin-4-one | | 2.28 (s, 3H), 1.47 (s, 9H), 1.24 (s, 3H), |
| | | | 1.17 (d, J = 6.8 Hz, 3H). |
| 146 | N-└6-(4-tert-butyl-5-chloro-2- | 382.11 | ¹H NMR (400 MHz, DMSO-d₆) δ 12.70 |
| | methyl-phenyl)-2-methyl-4- | 383 | (s, 1H), 8.80 (s, 1H), 7.46 (s, 1H), 7.46 (s, |
| | oxo-1H-pyridin-3- | | 1H), 6.65 (s, 1H), 3.07 (s, 3H), 2.46 (s, |
| | yl]methanesulfonamide | | 3H), 2.27 (s, 3H), 1.48 (s, 9H). |

For Compound 119 and Compound 120, the racemic OBn-precursor [4-benzyloxy-6-(4-tert-butyl-5-chloro-2-methyl-phenyl)-2-methyl-3-pyridyl]-imino-methyl-oxo-6-sulfane was purified by chiral SFC (ChiralPak IC column, 250×21.2 mm, 5 μm particle size, isocratic 70% CO₂/30% MeOH (20 mM NH₃) over 9 minutes, flow rate=70 mL/min, column temperature=40° C.) to provide the separated OBn-protected enantiomers:

Peak 1 (retention time 5.5 min) was isolated and debenzylated using standard hydrogenation conditions to provide Compound 119.

Peak 2 (retention time 7.0 min) was isolated and debenzylated using standard hydrogenation conditions to provide Compound 120.

Compound 127 was prepared from Intermediate A-5A and Compound 128 was prepared from Intermediate A-5B.

Compound 129 was prepared from Intermediate A-5A.

Compound 130 was prepared from Intermediate A-5A.

For Compound 131 and Compound 132, the racemic OBn-precursor [4-benzyloxy-6-[4-tert-butyl-2-methyl-5-(trifluoromethyl)phenyl]-2-methyl-3-pyridyl]-imino-methyl-oxo-6-sulfane was purified by chiral SFC (ChiralPak IC column, 250×21.2 mm, 5 μm particle size, isocratic 82% CO₂/18% MeOH (20 mM NH₃) over 10 minutes, flow rate=70 mL/min, column temperature=40° C.) to provide the separated OBn-protected enantiomers:

Peak 1 (retention time 6.0 min) was isolated and debenzylated using standard hydrogenation conditions to provide Compound 131.

Peak 2 (retention time 7.9 min) was isolated and debenzylated using standard hydrogenation conditions to provide Compound 132.

### Example 5

### 6-(4-tert-butyl-2-methyl-phenyl)-3-(hydroxymethyl)-2-methyl-1H-pyridin-4-one (147)

### Step 1: 6-(4-tert-butyl-2-methyl-phenyl)-3-(hydroxymethyl)-2-methyl-1H-pyridin-4-one (147)

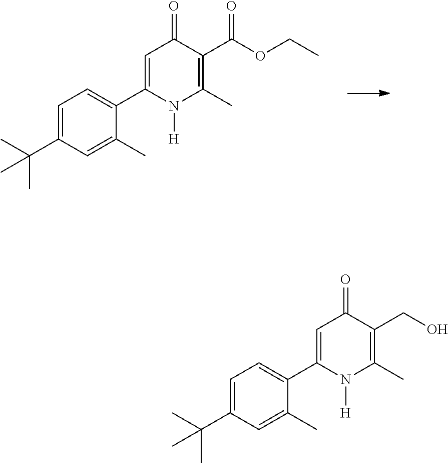

To a solution of ethyl 6-(4-tert-butyl-2-methyl-phenyl)-2-methyl-4-oxo-1H-pyridine-3-carboxylate (15 mg, 0.05 mmol) in THF (2 mL) was added solid LiBH₄ (10 mg, 0.46 mmol) at 0° C. The mixture was gradually warmed to room temperature and stirred for 16 h. The mixture was quenched with water and the aqueous layer was extracted with DCM, dried over magnesium sulfate, filtered and concentrated. The crude product was purified via reverse phase HPLC (CIS) using 1 to 60% acetonitrile in water to obtain 6-(4-tert-butyl-2-methyl-phenyl)-3-(hydroxymethyl)-2-methyl-1H-pyridin-4-one (147, 6 mg, 46%). ESI-MS m/z calc. 285.17, found 286.15 (M+1)⁺. ¹H NMR (500 MHz, DMSO-d₆) δ 11.18 (s, 1H), 7.37 (d, J=2.1 Hz, 1H), 7.33 (dd, J=7.9, 2.1 Hz, 1H), 7.23 (d, J=8.0 Hz, 1H), 5.98 (s, 1H), 4.85 (s, 1H), 4.41 (s, 2H), 2.31 (s, 3H), 2.27 (s, 3H), 1.31 (s, 9H) ppm.

### Example 6

### 6-(4-tert-butyl-2-methyl-phenyl)-3-(1-hydroxy-1-methyl-ethyl)-2-methyl-1H-pyridin-4-one (148)

### Step 1: 2-[6-(4-tert-butyl-2-methyl-phenyl)-4-[(4-methoxyphenyl)methoxy]-2-methyl-3-pyridyl]propan-2-ol

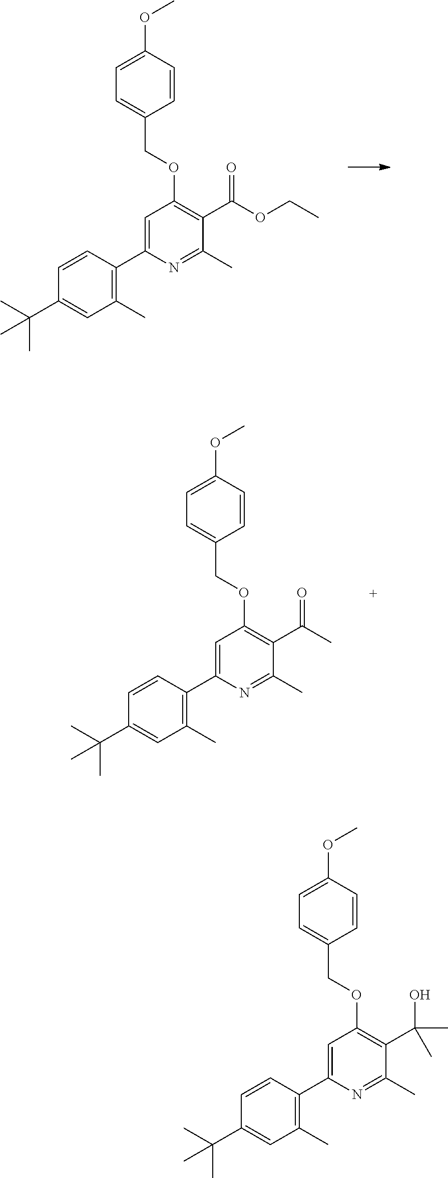

A solution of ethyl 6-(4-tert-butyl-2-methyl-phenyl)-4-[(4-methoxyphenyl)methoxy]-2-methyl-pyridine-3-carboxylate (250 mg, 0.56 mmol) in THF (5 mL) was treated with methyl magnesium bromide (600 L of 3 M, 1.8 mmol) at 0° C. under nitrogen atmosphere. The mixture was stirred at room temperature overnight and quenched with aqueous ammonium chloride solution. The mixture was extracted with ethyl acetate. The organic layer was dried over sodium sulfate, filtered, evaporated and purified by silica gel chromatography using 0 to 20% ethyl acetate in to give two products 1-[6-(4-tert-butyl-2-methyl-phenyl)-4-[(4-methoxyphenyl)methoxy]-2-methyl-3-pyridyl]ethanone (89.7 mg, 38%). ¹H NMR (400 MHz, CDCl₃) δ 7.32-7.26 (m, 5H), 6.91 (d, J=8.7 Hz, 2H), 6.86 (s, 1H), 5.07 (s, 2H), 3.82 (s, 3H), 2.54 (s, 3H), 2.49 (s, 3H), 2.31 (s, 3H), 1.33 (s, 9H) ppm. ESI-MS m/z calc. 417.23, found 418.7 (M+1)⁺ and 2-[6-(4-tert-butyl-2-methyl-phenyl)-4-[(4-methoxyphenyl)methoxy]-2-methyl-3-pyridyl]propan-2-ol (86.0 mg, 36%). ¹H NMR (400 MHz, CDCl₃) δ 7.34 (d, J=8.6 Hz, 2H), 7.30-7.26 (m, 3H), 6.93 (d, J=8.7 Hz, 2H), 6.89 (s, 1H), 5.07 (s, 2H), 4.36 (s, 1H), 3.83 (s, 3H), 2.74 (s, 3H), 2.34 (s, 3H), 1.71 (s, 6H), 1.33 (s, 9H) ppm. ESI-MS m/z calc. 433.26, found 434.7 (M+1)⁺.

### Step 2: 6-(4-tert-butyl-2-methyl-phenyl)-3-(1-hydroxy-1-methyl-ethyl)-2-methyl-1H-pyridin-4-one (148)

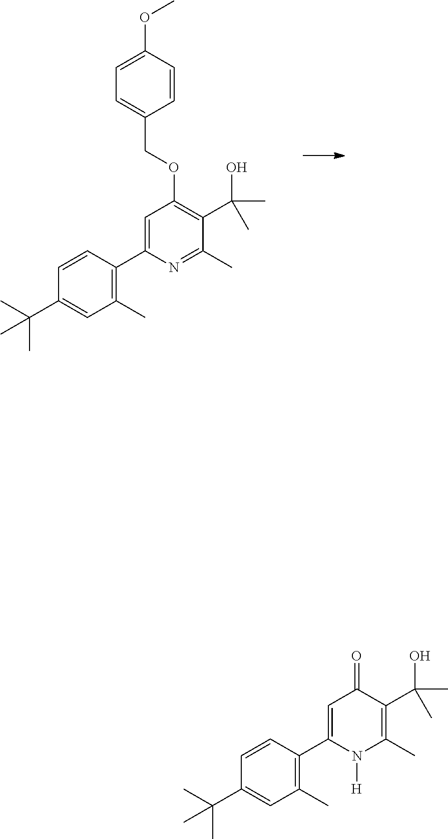 [SMILES: COc1ccc(COc2cc(-c3ccc(C(C)(C)C)cc3C)nc(C)c2C(C)(C)O)cc1 ; Cc1cc(C(C)(C)C)ccc1-c1cc(=O)c(C(C)(C)O)c(C)[nH]1]

A solution of 2-[6-(4-tert-butyl-2-methyl-phenyl)-4-[(4-methoxyphenyl)methoxy]-2-methyl-3-pyridyl]propan-2-ol (20 mg, 0.05 mmol) and 10% Pd/C (5 mg, 0.005 mmol) in methanol (1.5 mL) was sparged with hydrogen at 50° C. for 30 min. The mixture was then filtered and purified by reverse phase HPLC (C₁₈) using 1 to 99% acetonitrile in water containing 5 mM HCl to give 6-(4-tert-butyl-2-methyl-phenyl)-3-(1-hydroxy-1-methyl-ethyl)-2-methyl-1H-pyridin-4-one (148, 8.2 mg, 56%). ESI-MS m/z calc. 313.20, found 314.5 (M+1)⁺. ¹H NMR (400 MHz, DMSO-d₆) δ 13.59 (s, 1H), 7.46 (d, J=1.9 Hz, 1H), 7.42 (dd, J=8.0, 1.9 Hz, 1H), 7.36 (d, J=8.1 Hz, 1H), 7.16 (s, 1H), 2.81 (s, 3H), 2.27 (s, 3H), 1.64 (s, 6H), 1.32 (s, 9H) ppm.

### Example 7

### 3-acetyl-6-(4-tert-butyl-2-methyl-phenyl)-2-methyl-1H-pyridin-4-one (149)

### Step 1: 3-acetyl-6-(4-tert-butyl-2-methyl-phenyl)-2-methyl-1H-pyridin-4-one (149)

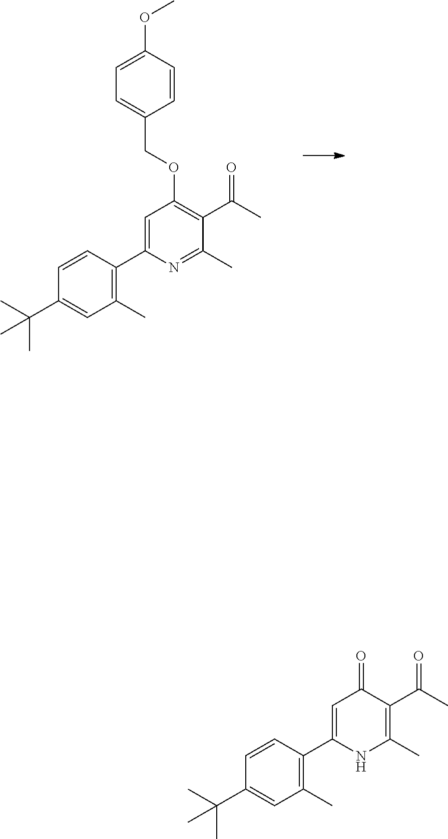 [SMILES: COc1ccc(COc2cc(-c3ccc(C(C)(C)C)cc3C)nc(C)c2C(C)=O)cc1 ; CC(=O)c1c(C)[nH]c(-c2ccc(C(C)(C)C)cc2C)cc1=O]

A solution of 1-[6-(4-tert-butyl-2-methyl-phenyl)-4-[(4-methoxyphenyl)methoxy]-2-methyl-3-pyridyl]ethanone (17 mg, 0.04 mmol) and Pd/C (5 mg of 10% w/w, 0.005 mmol) in methanol (1.5 mL) was sparged with hydrogen gas (10 mg, 4.96 mmol) at 50° C. for 30 min. The mixture was then filtered and purified by reverse phase HPLC (C₁₈) using 1 to 99% acetonitrile in water containing 5 mM HCl to give 3-acetyl-6-(4-tert-butyl-2-methyl-phenyl)-2-methyl-1H-pyridin-4-one (149, 6.8 mg, 56%). ESI-MS m/z calc. 297.17, found 298.5 (M+1)⁺. ¹H NMR (400 MHz, DMSO-d₆) δ 13.16 (s, 1H), 7.47-7.37 (m, 2H), 7.34 (d, J=8.0 Hz, 1H), 6.77 (s, 1H), 2.53 (s, 3H), 2.41 (s, 3H), 2.29 (s, 3H), 1.32 (s, 9H) ppm.

### Example 8

### rac-6-(4-tert-butyl-2-methyl-phenyl)-3-(1-hydroxyethyl)-2-methyl-1H-pyridin-4-one (150)

### Step 1: rac-6-(4-tert-butyl-2-methyl-phenyl)-3-(1-hydroxyethyl)-2-methyl-1H-pyridin-4-one (150)

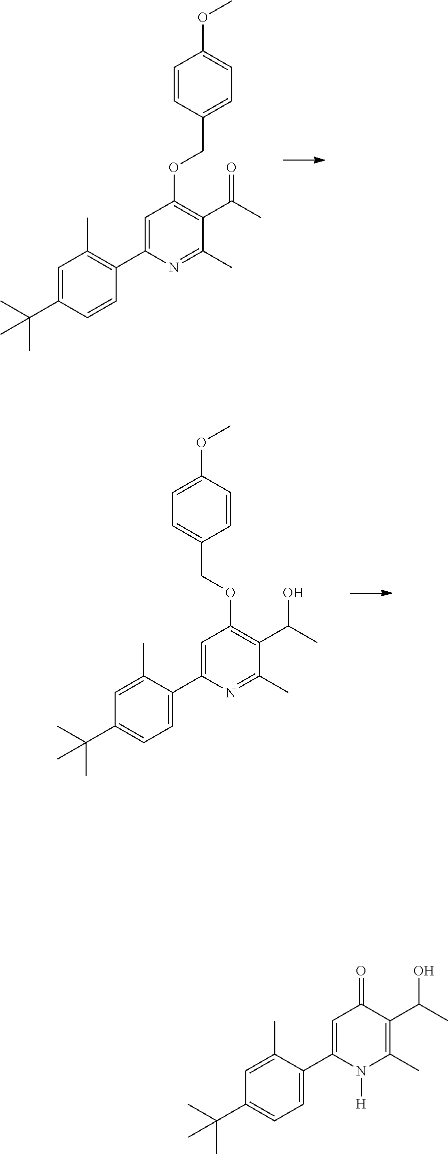 [SMILES: COc1ccc(COc2cc(-c3ccc(C(C)(C)C)cc3C)nc(C)c2C(C)=O)cc1 ; COc1ccc(COc2cc(-c3ccc(C(C)(C)C)cc3C)nc(C)c2C(C)O)cc1 ; Cc1cc(C(C)(C)C)ccc1-c1cc(=O)c(C(C)O)c(C)[nH]1]

A solution of 1-[6-(4-tert-butyl-2-methyl-phenyl)-4-[(4-methoxyphenyl)methoxy]-2-methyl-3-pyridyl]ethanone (66 mg, 0.16 mmol) in methanol (5 mL) was treated with sodium borohydride (6 mg, 0.16 mmol) and stirred at room temperature overnight. The mixture was then evaporated and purified by reverse phase HPLC (C₁₈) using 1 to 99% acetonitrile in water containing 5 mM HCl to give 1-[6-(4-tert-butyl-2-methyl-phenyl)-4-[(4-methoxyphenyl)methoxy]-2-methyl-3-pyridyl]ethanol (52.3 mg, 79%). ESI-MS m/z calc. 419.24, found 420.8 (M+1)⁺. The obtained intermediate (13 mg, 0.03 mmol) was treated with Pd/C (8.5 mg of 10% w/w, 0.008 mmol) in methanol (2 mL) and stirred under an atmosphere of hydrogen at 50° C. for 20 min. The mixture was filtered and purified by reverse phase HPLC (C₁₈) using 1 to 99% acetonitrile in water containing 5 mM HCl to give rac-6-(4-tert-butyl-2-methyl-phenyl)-3-(1-hydroxyethyl)-2-methyl-1H-pyridin-4-one (150, 5.3 mg, 11%). ESI-MS m/z calc. 299.18, found 300.5 (M+1)⁺. ¹H NMR (400 MHz, DMSO-d₆) δ 13.81 (s, 1H), 7.48-7.40 (m, 2H), 7.37 (d, J=8.0 Hz, 1H), 7.08 (d, J=10.8 Hz, 1H), 5.29-5.25 (m, 1H), 2.72 (s, 3H), 2.27 (s, 3H), 1.42 (d, J=6.7 Hz, 3H), 1.32 (s, 9H) ppm.

### Example 9

### 2-(4-tert-butyl-2-methyl-phenyl)-3-(hydroxymethyl)-5,6-dimethyl-1H-pyridin-4-one (151)

### Step 1: 2-(4-tert-butyl-2-methyl-phenyl)-3-(hydroxymethyl)-5,6-dimethyl-1H-pyridin-4-one

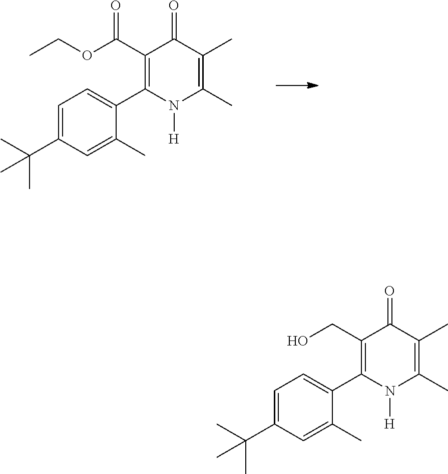 [SMILES: CCOC(=O)c1c(-c2ccc(C(C)(C)C)cc2C)[nH]c(C)c(C)c1=O ; Cc1cc(C(C)(C)C)ccc1-c1[nH]c(C)c(C)c(=O)c1CO]

A solution of ethyl 2-(4-tert-butyl-2-methyl-phenyl)-5,6-dimethyl-4-oxo-1H-pyridine-3-carboxylate (40 mg, 0.12 mmol) in THF (1.1 mL) was cooled to 0° C. followed by the addition of LiBH₄ (18 mg, 0.83 mmol). The reaction was warmed to room temperature overnight. The reaction was cooled back down to 0° C. and quenched with water, then saturated ammonium chloride, extracted with ethyl acetate (3×), washed with brine, dried with sodium sulfate, filtered, and concentrated. The product was purified by reverse phase HPLC (C₁₈) using 1 to 60% acetonitrile in water containing 5 mM HCl, followed by a second purification using silica gel chromatography using 0 to 20% methanol in DCM to afford 2-(4-tert-butyl-2-methyl-phenyl)-3-(hydroxymethyl)-5,6-dimethyl-1H-pyridin-4-one (151, 4.6 mg, 13%) as a white solid. ESI-MS m/z calc. 299.19, found 300.5 (M+1)⁺.

### Example 10

### 2-(4-tert-butyl-2-methyl-phenyl)-3-(methoxymethyl)-5,6-dimethyl-1H-pyridin-4-one (152)

### Step 1: [4-benzyloxy-2-(4-tert-butyl-2-methyl-phenyl)-5,6-dimethyl-3-pyridyl]methanol

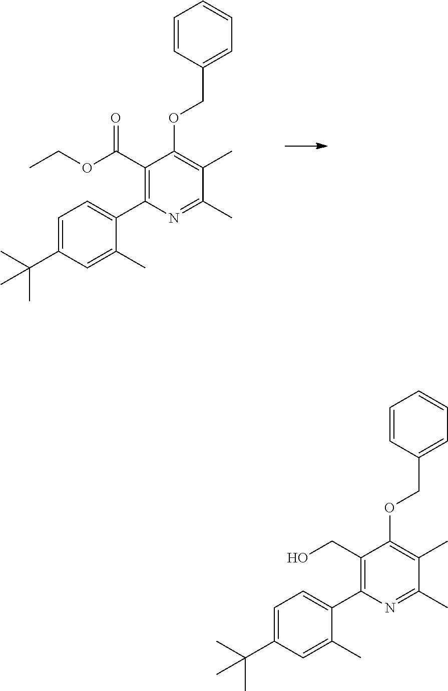 [SMILES: CCOC(=O)c1c(-c2ccc(C(C)(C)C)cc2C)nc(C)c(C)c1OCc1ccccc1 ; Cc1cc(C(C)(C)C)ccc1-c1nc(C)c(C)c(OCc2ccccc2)c1CO]

A vial equipped with a stir bar was charged with ethyl 4-benzyloxy-2-(4-tert-butyl-2-methyl-phenyl)-5,6-dimethyl-pyridine-3-carboxylate (85 mg, 0.2 mmol) and THF (2.0 mL) then cooled to −40° C. LiAlH₄ (400 μL of 2.0 M, 0.8 mmol) was added dropwise and the reaction was gradually warmed to room temperature and stirred for 5 h. The reaction was cooled to 0° C. and quenched by the addition of water (30 μL), then 4M NaOH (30 μL), then water (90 μL) and allowed to stir at room temperature for 15 min. The reaction was then filtered over Celite® with ethyl acetate and concentrated in vacuo. Purification by silica gel column chromatography with 0 to 40% ethyl acetate in hexanes gave [4-benzyloxy-2-(4-tert-butyl-2-methyl-phenyl)-5,6-dimethyl-3-pyridyl]methanol (47 mg, 58%) as a clear oil. ESI-MS m/z calc. 389.23, found 390.5 (M+1)⁺.

### Step 2: 4-benzyloxy-2-(4-tert-butyl-2-methyl-phenyl)-3-(methoxymethyl)-5,6-dimethyl-pyridine

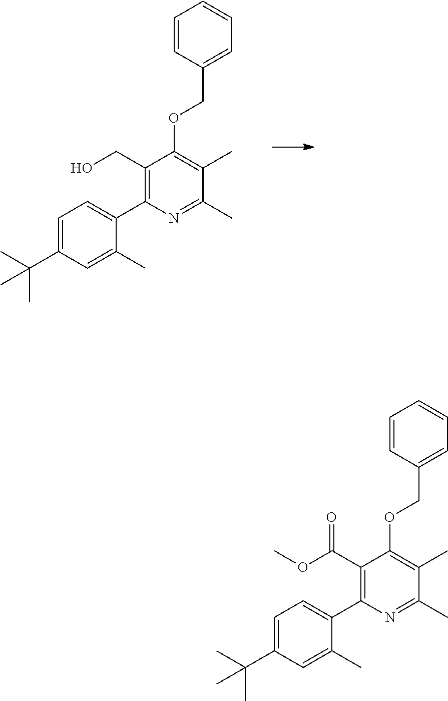 [SMILES: Cc1cc(C(C)(C)C)ccc1-c1nc(C)c(C)c(OCc2ccccc2)c1CO ; COC(=O)c1c(-c2ccc(C(C)(C)C)cc2C)nc(C)c(C)c1OCc1ccccc1]

To a solution of [4-benzyloxy-2-(4-tert-butyl-2-methyl-phenyl)-5,6-dimethyl-3-pyridyl]methanol (47 mg, 0.11 mmol) in DMF (1.2 mL) at 0° C. was added sodium hydride (6.9 mg of 60% w/w, 0.17 mmol) and the mixture was warmed to room temperature for 30 min. The reaction was then cooled to 0° C. and iodomethane (22 μL, 0.35 mmol) was added. The reaction was gradually warmed to room temperature and stirred overnight. The reaction was quenched with saturated ammonium chloride, extracted s with ethyl acetate (3×), washed with brine, dried with sodium sulfate, filtered, and concentrated. The crude material was purified by silica gel column chromatography with 0 to 30% ethyl acetate in hexanes to afford 4-benzyloxy-2-(4-tert-butyl-2-methyl-phenyl)-3-(methoxymethyl)-5,6-dimethyl-pyridine (39.4 mg, 85%) as a clear oil. ESI-MS m/z calc. 403.25, found 404.0 (M+1)⁺.

### Step 3: 2-(4-tert-butyl-2-methyl-phenyl)-3-(methoxymethyl)-5,6-dimethyl-1H-pyridin-4-one (152)

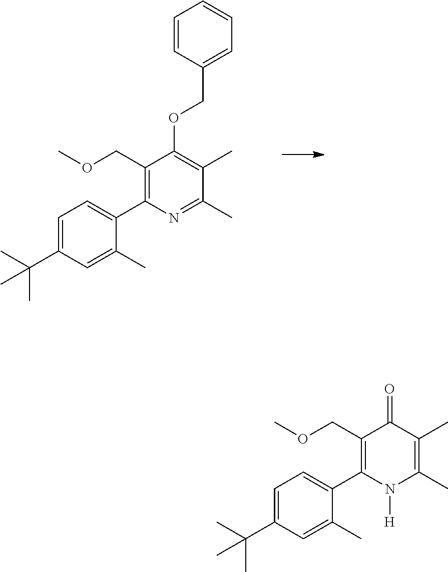 [SMILES: COCc1c(-c2ccc(C(C)(C)C)cc2C)nc(C)c(C)c1OCc1ccccc1 ; COCc1c(-c2ccc(C(C)(C)C)cc2C)[nH]c(C)c(C)c1=O]

Palladium on carbon (6.5 mg of 10% w/w, 0.006 mmol) was added to a solution of 4-benzyloxy-2-(4-tert-butyl-2-methyl-phenyl)-3-(methoxymethyl)-5,6-dimethyl-pyridine (40 mg, 0.1 mmol) in methanol (1.0 mL) under nitrogen then the reaction was sparged with hydrogen for 5 min and left to stir under a hydrogen atmosphere for 20 min. The reaction was filtered through Celite®, rinsed with methanol and concentrated in vacuo. The crude material was purified by silica gel flash column chromatography with 0 to 15% methanol in DCM to afford 2-(4-tert-butyl-2-methyl-phenyl)-3-(methoxymethyl)-5,6-dimethyl-1H-pyridin-4-one (152, 25 mg, 81%) as a white solid. ESI-MS m/z calc. 313.20, found 314.6 (M+1)⁺. ¹H NMR (400 MHz, DMSO-d₆) δ 11.02 (s, 1H), 7.36 (s, 1H), 7.31 (d, J=8.1 Hz, 1H), 7.17 (d, J=7.9 Hz, 1H), 4.00 (d, J=9.8 Hz, 1H), 3.74 (d, J=9.8 Hz, 1H), 3.01 (s, 3H), 2.20 (s, 3H), 2.12 (s, 3H), 1.88 (s, 3H), 1.31 (s, 9H) ppm.

### Example 11

### 6-(4-tert-butyl-2-methyl-phenyl)-3-isopropyl-2-methyl-1H-pyridin-4-one (153)

### Step 1: 6-(4-tert-butyl-2-methyl-phenyl)-3-isopropyl-2-methyl-1H-pyridin-4-one (153)

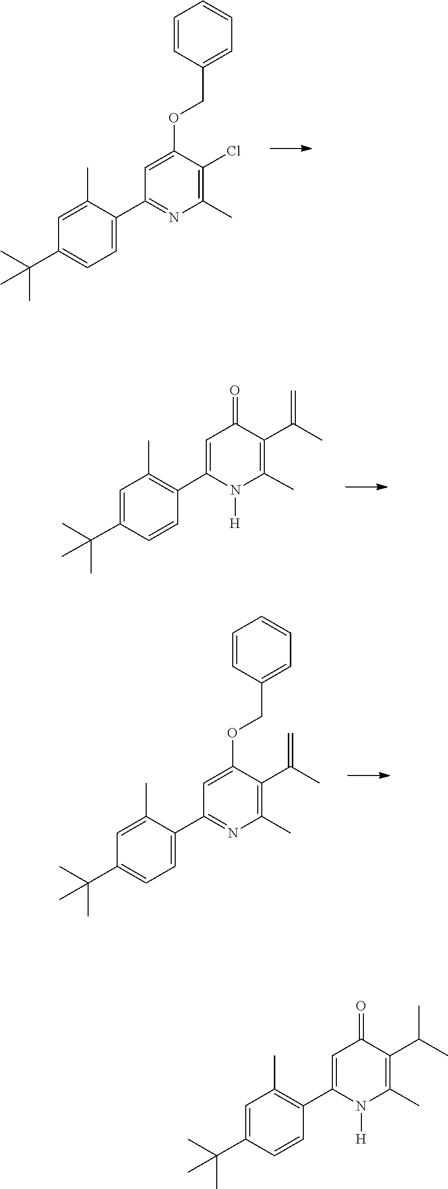 [SMILES: Cc1cc(C(C)(C)C)ccc1-c1cc(OCc2ccccc2)c(Cl)c(C)n1 ; C=C(C)c1c(C)[nH]c(-c2ccc(C(C)(C)C)cc2C)cc1=O ; C=C(C)c1c(OCc2ccccc2)cc(-c2ccc(C(C)(C)C)cc2C)nc1C ; Cc1cc(C(C)(C)C)ccc1-c1cc(=O)c(C(C)C)c(C)[nH]1]

In a 2 mL microwave vial a solution of 4-benzyloxy-6-(4-tert-butyl-2-methyl-phenyl)-3-chloro-2-methyl-pyridine (40 mg, 0.11 mmol), potassium carbonate (44 mg. 0.32 mmol), 2-isopropenyl-4,4,5,5-tetramethyl-1,3,2-dioxaborolane (27 mg, 0.16 mmol) and Sphos palladacycle Gen 3 (9 mg, 0.01 mmol) in EtOH (1 mL) and water (0.3 μL) was sparged with nitrogen, sealed and irradiated at 100° C. for 10 min. The mixture was filtered and purified by reverse phase HPLC (C₁₈) using 2 to 80% acetonitrile in water containing 5 mM HCl to give 6-(4-tert-butyl-2-methyl-phenyl)-3-isopropenyl-2-methyl-1H-pyridin-4-one (10.6 mg, 34%). ESI-MS m/z calc. 295.19, found 296.6 (M+1)⁺ and 4-benzyloxy-6-(4-tert-butyl-2-methyl-phenyl)-3-isopropenyl-2-methyl-pyridine (15.0 mg, 37%). ESI-MS m/z calc. 385.24, found 386.7 (M+1)⁺. The obtained intermediates were combined in methanol (3 mL), treated with Pd/C (12 mg of 10% w/w, 0.01 mmol) and sparged with hydrogen gas. The mixture was stirred at 50° C. under hydrogen atmosphere for 1 h. The mixture was filtered and purified by reverse phase HPLC (C₁₈) using 1 to 99% acetonitrile in water containing 5 mM HCl to give 6-(4-tert-butyl-2-methyl-phenyl)-3-isopropyl-2-methyl-1H-pyridin-4-one (153, 14.4 mg, 46%). ESI-MS m/z calc. 297.21, found 298.5 (M+1)⁺. ¹H NMR (400 MHz, DMSO-d₆) δ 13.86 (s, 1H), 7.46 (d, J=1.9 Hz, 1H), 7.42 (dd, J=8.1, 2.0 Hz, 1H), 7.36 (d, J=8.0 Hz, 1H), 7.21 (s, 1H), 3.26 (q, J=7.0 Hz, 1H), 2.64 (s, 3H), 2.27 (s, 3H), 1.35 (s, 3H), 1.33 (d, J=2.6 Hz, 12H) ppm.

### Example 12

### 3-(aminomethyl)-6-(4-tert-butyl-2-methyl-phenyl)-2-methyl-1H-pyridin-4-one (154)

### Step 1: N-benzyl-N-[(4,6-dichloro-2-methyl-3-pyridyl)methyl]-1-phenyl-methanamine

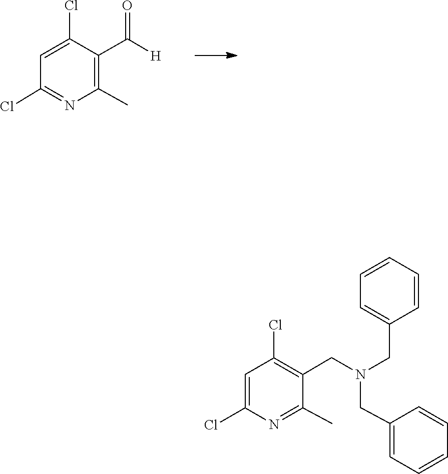 [SMILES: Cc1nc(Cl)cc(Cl)c1C=O ; Cc1nc(Cl)cc(Cl)c1CN(Cc1ccccc1)Cc1ccccc1]

A solution of 4,6-dichloro-2-methyl-pyridine-3-carbaldehyde (680 mg, 3.58 mmol) and N-benzyl-1-phenyl-methanamine (1.5 g, 7.60 mmol) in DCE (5 mL) was stirred at room temperature for 30 min. Methanol (10 mL) was added, and the mixture was cooled using an ice bath. Sodium triacetoxyborohydride (2.3 g, 10.85 mmol) was added portion wise and the mixture was stirred at room temperature overnight. The mixture was quenched with water and extracted with ethyl acetate (3×20 mL). The organic layer was dried over sodium sulfate, filtered and evaporated. It was purified by silica gel chromatography using 0 to 10% ethyl acetate in hexanes to give N-benzyl-N-[(4,6-dichloro-2-methyl-3-pyridyl)methyl]-1-phenyl-methanamine (208 mg, 16%). ESI-MS m/z calc. 370.10, found 373.2 (M+1)⁺. ¹H NMR (400 MHz, CDCl₃) δ 7.45-7.31 (m, 1H), 7.26 (s, 1H), 3.81 (s, 2H), 3.61 (s, 4H), 2.55 (s, 3H) ppm.

### Step 2: N-benzyl-N-[(4-benzyloxy-6-chloro-2-methyl-3-pyridyl)methyl]-1-phenyl-methanamine

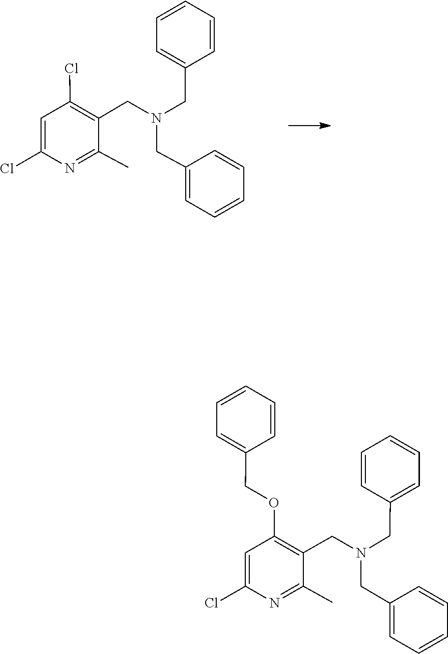 [SMILES: Cc1nc(Cl)cc(Cl)c1CN(Cc1ccccc1)Cc1ccccc1 ; Cc1nc(Cl)cc(OCc2ccccc2)c1CN(Cc1ccccc1)Cc1ccccc1]

A solution of sodium hydride (33 mg of 60% w/w, 0.83 mmol) in DMF (3 mL) was treated with a solution of N-benzyl-N-[(4,6-dichloro-2-methyl-3-pyridyl)methyl]-1-phenyl-methanamine (205 mg, 0.55 mmol) and benzyl alcohol (78 mg, 0.72 mmol) in small amount of DMF. The mixture was stirred at room temperature for 2 h, quenched with small amount of methanol, filtered and purified by reverse phase HPLC (C₁₈) using 1 to 99% acetonitrile in water containing 5 mM HCl to give N-benzyl-N-[(4-benzyloxy-6-chloro-2-methyl-3-pyridyl)methyl]-1-phenyl-methanamine (140 mg, 57%). ESI-MS m/z calc. 442.18, found 444.4 (M+2)⁺. ¹H NMR (400 MHz, CDCl₃) δ 7.48-7.37 (m, 5H), 7.27-7.19 (m, 10H), 6.66 (s, 1H), 5.06 (s, 2H), 3.57 (s, 2H), 3.46 (s, 4H), 2.36 (s, 3H) ppm.

### Step 3: 3-(aminomethyl)-6-(4-tert-butyl-2-methyl-phenyl)-2-methyl-1H-pyridin-4-one (154)

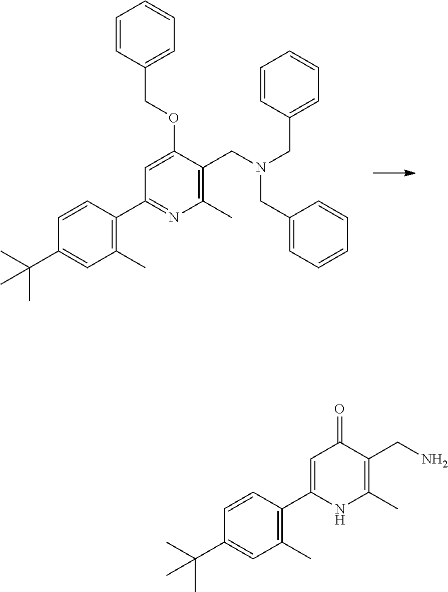 [SMILES: Cc1cc(C(C)(C)C)ccc1-c1cc(OCc2ccccc2)c(CN(Cc2ccccc2)Cc2ccccc2)c(C)n1 ; Cc1cc(C(C)(C)C)ccc1-c1cc(=O)c(CN)c(C)[nH]1]

N-benzyl-N-[(4-benzyloxy-6-chloro-2-methyl-3-pyridyl)methyl]-1-phenyl-methanamine (15 mg, 0.03 mmol), ammonium formate (15 mg, 0.24 mmol) and Pd/C (5 mg of 10% w/w, 0.005 mmol) in EtOH (1.5 mL) was stirred at 80° C. for 1 h. The mixture was filtered and purified by reverse phase HPLC (C₁₈) using 1 to 99% acetonitrile in water containing 5 mM HCl to give 3-[(benzylamino)methyl]-6-(4-tert-butyl-2-methyl-phenyl)-2-methyl-1H-pyridin-4-one (Hydrochloride salt) (15.4 mg, 12%,). ESI-MS m/z calc. 374.23, found 375.6 (M+1)⁺. ¹H NMR (400 MHz, DMSO-d₆) δ 12.44 (s, 1H), 9.31 (s, 2H), 7.60-7.53 (m, 2H), 7.49-7.35 (m, 5H), 7.27 (d, J=8.0 Hz, 1H), 6.47 (s, 1H), 4.24 (s, 2H), 3.99 (s, 2H), 2.42 (s, 3H), 2.26 (s, 3H), 1.31 (s, 9H). and 3-(aminomethyl)-6-(4-tert-butyl-2-methyl-phenyl)-2-methyl-1H-pyridin-4-one (hydrochloride salt) (154, 8 mg, 77%). ESI-MS m/z calc. 284.19, found 285.3 (M+1)⁺. ¹H NMR (400 MHz, DMSO-d₆) δ 13.27 (s, 1H), 8.24 (br s, 3H), 7.45 (s, 1H), 7.43-7.39 (m, 1H), 7.32 (d, J=8.0 Hz, 1H), 6.90 (s, 1H), 3.98-3.94 (m, 2H), 2.63 (s, 3H), 2.27 (s, 3H), 1.32 (s, 9H).

### Example 13

### (2R)-2-amino-N-[6-(4-tert-butyl-2-methyl-phenyl)-2-methyl-4-oxo-1H-pyridin-3-yl]propanamide (155)

### Step 1: (2R)-2-amino-N-[6-(4-tert-butyl-2-methyl-phenyl)-2-methyl-4-oxo-1H-pyridin-3-yl]propanamide (155)

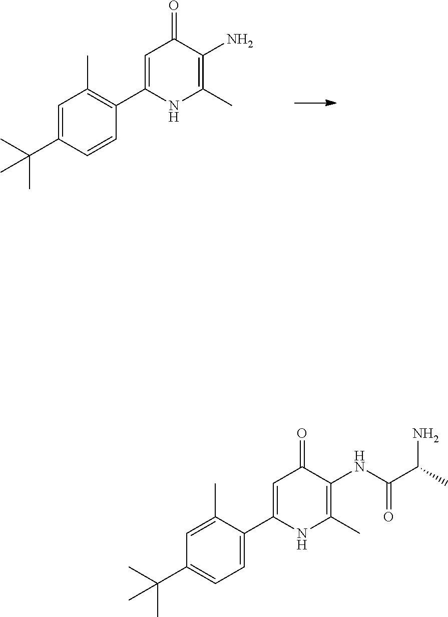 [SMILES: Cc1cc(C(C)(C)C)ccc1-c1cc(=O)c(N)c(C)[nH]1 ; Cc1cc(C(C)(C)C)ccc1-c1cc(=O)c(NC(=O)[C@@H](C)N)c(C)[nH]1]

A vial charged with 3-amino-6-(4-tert-butyl-2-methyl-phenyl)-2-methyl-1H-pyridin-4-one (50 mg, 0.19 mmol), (2R)-2-(tert-butoxycarbonylamino)propanoic acid (35 mg, 0.19 mmol), DIEA (48 mg, 65 μL, 0.37 mmol) and HATU (92 mg, 0.24 mmol) in DMF (1 mL) The aqueous layer was extracted with DCM (2×). The combined organic layer was dried over magnesium sulfate, filtered and concentrated. The crude material was taken up in DCM and HCl in 1,4-dioxane (230 μL of 4 M, 0.92 mmol) and was stirred for 1 h. The solvent was evaporated and the crude material was taken up in DMSO and purified via reverse phase HPLC (C₁₈) using 1 to 99% acetonitrile in water containing 5 mM HCl to obtain (2R)-2-amino-N-[6-(4-tert-butyl-2-methyl-phenyl)-2-methyl-4-oxo-1H-pyridin-3-yl]propanamide (hydrochloride salt) (155, 35 mg, 55%). ESI-MS m/z calc. 341.21, found 342.2 (M+1)⁺. ¹H NMR (400 MHz, CD₃OD) δ 7.51-7.43 (m, 2H), 7.37 (d, J=8.0 Hz, 1H), 7.08 (s, 1H), 4.29 (q, J=7.1 Hz, 1H), 3.66 (s, 3H), 2.56 (s, 3H), 2.35 (s, 3H), 1.71 (d, J=7.1 Hz, 3H), 1.36 (s, 9H) ppm.

### Example 14

### (2S)-2-amino-N-[6-(4-tert-butyl-2-methyl-phenyl)-2-methyl-4-oxo-1H-pyridin-3-yl]propanamide (156)

### Step 1: (2S)-2-amino-N-[6-(4-tert-butyl-2-methyl-phenyl)-2-methyl-4-oxo-1H-pyridin-3-yl]propanamide (156)

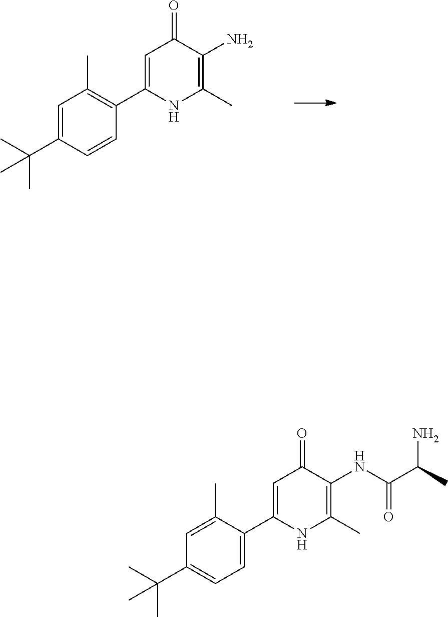 [SMILES: Cc1cc(C(C)(C)C)ccc1-c1cc(=O)c(N)c(C)[nH]1 ; Cc1cc(C(C)(C)C)ccc1-c1cc(=O)c(NC(=O)[C@H](C)N)c(C)[nH]1]

(2S)-2-amino-N-[6-(4-tert-butyl-2-methyl-phenyl)-2-methyl-4-oxo-1H-pyridin-3-yl]propanamide (hydrochloride salt) (156) was prepared from (2S)-2-(tert-butoxycarbonylamino)propanoic acid using procedure analogous to that found in Example 13. ESI-MS m/z calc. 341.21, found 342.2 (M+1)⁺.

### Example 15

### N-[6-(4-tert-butyl-2-methyl-phenyl)-2-methyl-4-oxo-1H-pyridin-3-yl]acetamide (157)

### Step 1: N-[6-(4-tert-butyl-2-methyl-phenyl)-2-methyl-4-oxo-1H-pyridin-3-yl]acetamide (157)

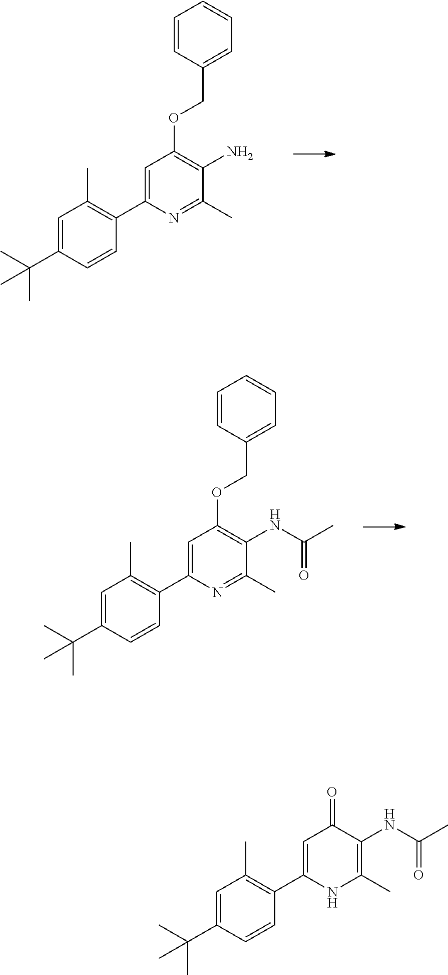

Acetyl chloride in DCM (100 μL of 1 M, 0.1 mmol) was added dropwise to a solution of 4-benzyloxy-6-(4-tert-butyl-2-methyl-phenyl)-2-methyl-pyridin-3-amine (OBn precursor of Compound 114, 30 mg, 0.08 mmol) in DCM (1 mL) cooled in a dry ice bath. The cold bath was removed, and the solution stirred at room temperature for 1 h. Then the mixture was concentrated in vacuo, and the residue was poured onto water (5 mL) and extracted with ethyl acetate (2×5 mL). The combined organic layers were washed with saturated sodium bicarbonate solution, brine, dried over magnesium sulfate, filtered, and concentrated under vacuum to obtain an oil that was purified via silica gel column chromatography using 0 to 30% ethyl acetate in hexanes to obtain N-[4-benzyloxy-6-(4-tert-butyl-2-methyl-phenyl)-2-methyl-3-pyridyl]acetamide (20 mg, 60%).

The N-[4-benzyloxy-6-(4-tert-butyl-2-methyl-phenyl)-2-methyl-3-pyridyl]acetamide was then taken up in ethanol (1 mL) and stirred with Pd/C (6 mg, 0.06 mmol) under an atmosphere of hydrogen for 30 min. The mixture was filtered through a plug of Celite® and washed with methanol. Purification by reverse phase chromatography (C₁₈) using 1 to 99% acetonitrile in water containing 5 mM HCl provided N-[6-(4-tert-butyl-2-methyl-phenyl)-2-methyl-4-oxo-1H-pyridin-3-yl]acetamide (157, 11 mg, 41%). ESI-MS m/z calc. 312.18, found 313.13 (M+1)⁺. ¹H NMR (400 MHz, DMSO-d₆) δ 14.17 (s, 1H), 9.74 (s, 1H), 7.50-7.36 (m, 3H), 7.21 (s, 1H), 2.44 (s, 3H), 2.29 (s, 3H), 2.11 (s, 3H), 1.33 (s, 9H) ppm.

### Example 16

### (2S)-2-amino-N-[[6-(4-tert-butyl-2-methyl-phenyl)-2-methyl-4-oxo-1H-pyridin-3-yl]methyl]propanamide (158)

### Step 1: (2S)-2-amino-N-[[6-(4-tert-butyl-2-methyl-phenyl)-2-methyl-4-oxo-1H-pyridin-3-yl]methyl]propanamide (158)

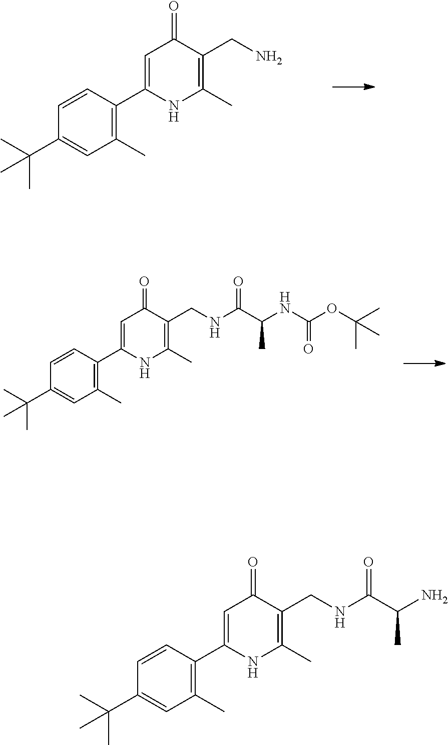

A solution of (2S)-2-(tert-butoxycarbonylamino)propanoic acid (17 mg, 0.09 mmol), DIPEA (25 mg, 0.19 mmol) and HATU (40 mg, 0.1 mmol) in DMF (1 mL) was stirred at room temperature for 10 min and it was added to 3-(aminomethyl)-6-(4-tert-butyl-2-methyl-phenyl)-2-methyl-1H-pyridin-4-one (hydrochloride salt) (29 mg, 0.09 mmol). The mixture was stirred at room temperature for 2 h, filtered and purified by reverse phase HPLC (C₁₈) using 1 to 99% acetonitrile in water containing 5 mM HCl to give tert-butyl N-[(1S)-2-[[6-(4-tert-butyl-2-methyl-phenyl)-2-methyl-4-oxo-1H-pyridin-3-yl]methylamino]-1-methyl-2-oxo-ethyl]carbamate (4.7 mg, 12%). ESI-MS m/z calc. 455.28, found 456.5 (M+1)⁺.

The obtained material was treated with 1,4-dioxane solution of HCl (500 L of 6 M, 3 mmol) and methanol (1 mL). The mixture was stirred at room temperature for 2 h and purified by reverse phase HPLC (C₁₈) using 1 to 99% acetonitrile in water containing 5 mM HCl to give (2S)-2-amino-N-[[6-(4-tert-butyl-2-methyl-phenyl)-2-methyl-4-oxo-1H-pyridin-3-yl]methyl]propanamide (hydrochloride salt) (158, 20.0 mg, 56%). ESI-MS m/z calc. 355.23, found 356.4 (M+1)⁺. ¹H NMR (400 MHz, DMSO-d₆) δ 13.76 (s, 1H), 8.80 (d, J=5.2 Hz, 1H), 8.17 (d, J=5.4 Hz, 3H), 7.47 (d, J=1.9 Hz, 1H), 7.42 (dd, J=8.0, 2.0 Hz, 1H), 7.34 (d, J=8.0 Hz, 1H), 7.16 (s, 1H), 4.39-4.27 (m, 2H), 3.83 (t, J=6.3 Hz, 1H), 2.67 (s, 3H), 2.27 (s, 3H), 1.32 (s, 9H) ppm.

### Example 17

### (2S)—N-[[6-(4-tert-butyl-2,5-dimethyl-phenyl)-2-methyl-4-oxo-1H-pyridin-3-yl]methyl]-2-(dimethylamino)-N-methyl-propanamide (159)

### Step 1: N-[(4,6-dichloro-2-methyl-3-pyridyl)methyl]-1-phenyl-methanamine

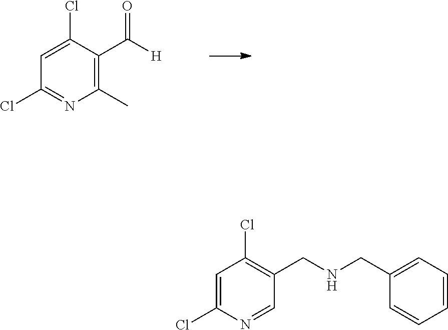 [SMILES: Cc1nc(Cl)cc(Cl)c1C=O ; Clc1cc(Cl)c(CNCc2ccccc2)cn1]

A solution of 4,6-dichloro-2-methyl-pyridine-3-carbaldehyde (300 mg, 1.58 mmol) and benzyl amine (220 mg, 2.05 mmol) in DCE (3 mL) was stirred at room temperature for 30 min. Methanol (5 mL) was added and the mixture was cooled using an ice bath and sodium triacetoxyborohydride was added portion wise and the mixture was stirred at room temperature overnight. The reaction was quenched with water and extracted with ethyl acetate (3×20 mL). The organic layer was dried over sodium sulfate, filtered, evaporated and purified by reverse phase HPLC (C₁₈) using 1 to 99% acetonitrile in water containing 5 mM HCl to give N-[(4,6-dichloro-2-methyl-3-pyridyl)methyl]-1-phenyl-methanamine (hydrochloride salt) (133.1 mg, 27%). ESI-MS m/z calc. 280.05, found 281.0 (M+1)⁺. ¹H NMR (400 MHz, DMSO-d₆) δ 9.63 (s, 2H), 7.75 (s, 1H), 7.62 (dd, J=7.4, 2.2 Hz, 2H), 7.47 (dd, J=7.2, 4.8 Hz, 3H), 4.33 (t, J=5.2 Hz, 2H), 4.20 (t, J=5.9 Hz, 2H), 2.59 (s, 3H) ppm.

### Step 2: N-[(4,6-dichloro-2-methyl-3-pyridyl)methyl]-N-methyl-1-phenyl-methanamine

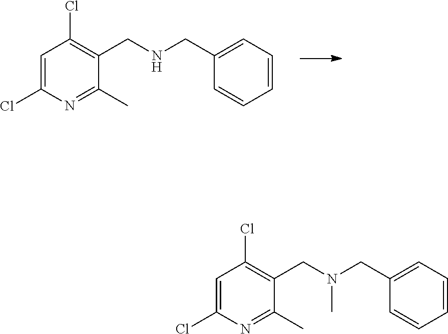 [SMILES: Cc1nc(Cl)cc(Cl)c1CNCc1ccccc1 ; Cc1nc(Cl)cc(Cl)c1CN(C)Cc1ccccc1]

A solution of N-[(4,6-dichloro-2-methyl-3-pyridyl)methyl]-1-phenyl-methanamine (133 mg, 0.47 mmol) in DMF (3 mL) was treated with sodium hydride (38 mg of 60% w/w, 0.94 mmol) followed by MeI (90 μL, 1.42 mmol) at 0° C. The mixture was stirred at 60° C. for 1 h and quenched with small amount of methanol, filtered and purified by reverse phase HPLC (C₁₈) using 1 to 99% acetonitrile in water containing 5 mM HCl to give N-[(4,6-dichloro-2-methyl-3-pyridyl)methyl]-N-methyl-1-phenyl-methanamine (103.3 mg, 74%). ESI-MS m/z calc. 294.07, found 295.1 (M+1)⁺.

### Step 3: N-[(4-benzyloxy-6-chloro-2-methyl-3-pyridyl)methyl]-N-methyl-1-phenyl-methanamine

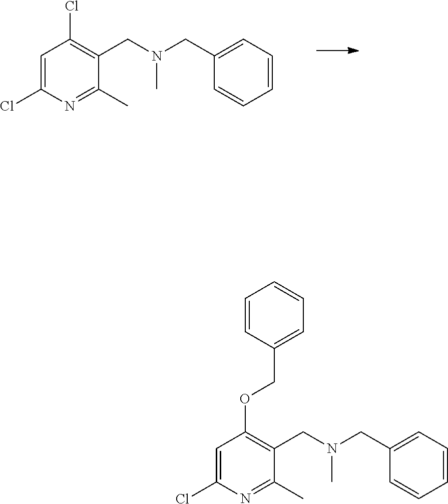 [SMILES: Cc1nc(Cl)cc(Cl)c1CN(C)Cc1ccccc1 ; Cc1nc(Cl)cc(OCc2ccccc2)c1CN(C)Cc1ccccc1]

To a suspension of sodium hydride (22 mg of 60% w/w, 0.55 mmol) in DMF (1.5 mL) was added a solution of N-[(4,6-dichloro-2-methyl-3-pyridyl)methyl]-N-methyl-1-phenyl-methanamine (103 mg, 0.35 mmol) and benzyl alcohol (51 mg, 0.47 mmol) dropwise at 0° C. The mixture was then stirred at 70° C. for 2 h, quenched with small amount of methanol, filtered and purified by reverse phase HPLC (C₁₈) using 1 to 99% acetonitrile in water containing 5 mM HCl to give N-[(4-benzyloxy-6-chloro-2-methyl-3-pyridyl)methyl]-N-methyl-1-phenyl-methanamine (52.3 mg, 41%). ESI-MS m/z calc. 366.15, found 367.3 (M+1)⁺.

### Step 4: 6-(4-tert-butyl-2,5-dimethyl-phenyl)-2-methyl-3-(methylaminomethyl)-1H-pyridin-4-one

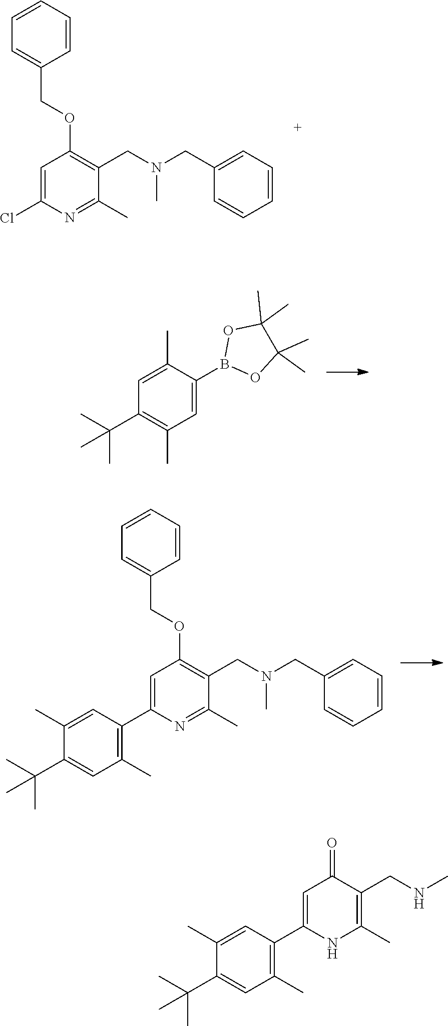 [SMILES: Cc1nc(Cl)cc(OCc2ccccc2)c1CN(C)Cc1ccccc1 ; Cc1cc(C(C)(C)C)c(C)cc1B1OC(C)(C)C(C)(C)O1 ; Cc1cc(C(C)(C)C)c(C)cc1-c1cc(OCc2ccccc2)c(CN(C)Cc2ccccc2)c(C)n1 ; CNCc1c(C)[nH]c(-c2cc(C)c(C(C)(C)C)cc2C)cc1=O]

In a 2 mL microwave vial a solution of N-[(4-benzyloxy-6-chloro-2-methyl-3-pyridyl)methyl]-N-methyl-1-phenyl-methanamine (52 mg, 0.14 mmol), potassium carbonate (41 mg, 0.3 mmol), 2-(4-tert-butyl-2,5-dimethyl-phenyl)-4,4,5,5-tetramethyl-1,3,2-dioxaborolane (63 mg, 0.22 mmol) and Pd(dppf)Cl₂·DCM (6 mg, 0.007 mmol) in EtOH (1 mL) and water (0.3 mL) was sparged with nitrogen, sealed and irradiated at 100° C. for 10 min. The mixture was filtered and purified by reverse phase HPLC (C₁₈) using 1 to 99% acetonitrile in water containing 5 mM HCl to give N-[[4-benzyloxy-6-(4-tert-butyl-2,5-dimethyl-phenyl)-2-methyl-3-pyridyl]methyl]-N-methyl-1-phenyl-methanamine (34.2 mg, 49%). ESI-MS m/z calc. 492.31, found 493.6 (M+1)⁺.

The obtained material was dissolved in EtOH (3 mL) and Pd/C (16 mg of 10% w/w, 0.015 mmol) and ammonium formate (35 mg, 0.55 mmol) were added. The mixture was stirred at 80° C. for 2 h. The mixture was filtered and concentrated to give 6-(4-tert-butyl-2,5-dimethyl-phenyl)-2-methyl-3-(methylaminomethyl)-1H-pyridin-4-one (20 mg, 45%). ESI-MS m/z calc. 312.22, found 313.6 (M+1)⁺.

### Step 5: (2S)—N-[[6-(4-tert-butyl-2,5-dimethyl-phenyl)-2-methyl-4-oxo-1H-pyridin-3-yl]methyl]-2-(dimethylamino)-N-methyl-propanamide (159)

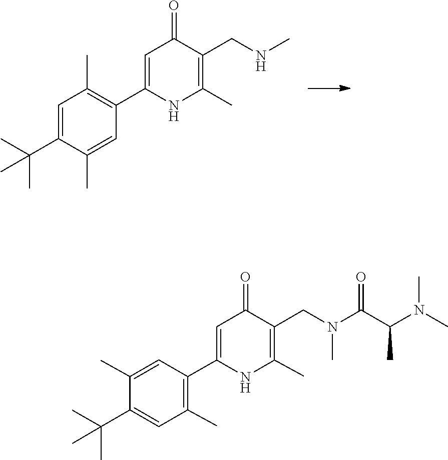

A solution of (2S)-2-(dimethylamino)propanoic acid (5 mg, 0.043 mmol), DIPEA (12 mg, 0.09 mmol) and HATU (14 mg, 0.04 mmol) in DMF (1 mL) was stirred at room temperature for 10 min and added to 6-(4-tert-butyl-2,5-dimethyl-phenyl)-2-methyl-3-(methylaminomethyl)-1H-pyridin-4-one (10 mg, 0.032 mmol). The mixture was stirred at room temperature for 2 h, filtered and purified by reverse phase HPLC (C₁₈) using 1 to 99% acetonitrile in water containing 5 mM HCl to give (2S)—N-[[6-(4-tert-butyl-2,5-dimethyl-phenyl)-2-methyl-4-oxo-1H-pyridin-3-yl]methyl]-2-(dimethylamino)-N-methyl-propanamide (hydrochloride salt) (159, 6.2 mg, 42%). ESI-MS m/z calc. 411.29, found 412.6 (M+1)⁺.

### Example 18

### (2S)-2-amino-N-[2-[6-(4-tert-butyl-2-methyl-phenyl)-2-methyl-4-oxo-1H-pyridin-3-yl]ethyl]propanamide (160)

### Step 1: 4-benzyloxy-6-(4-tert-butyl-2-methyl-phenyl)-2-methyl-pyridine-3-carbaldehyde

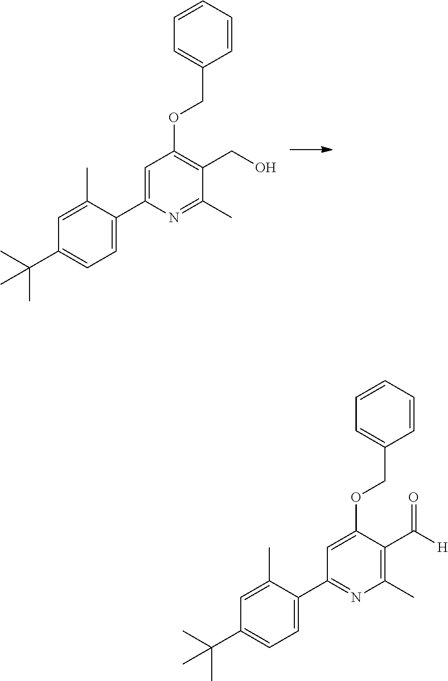 [SMILES: Cc1cc(C(C)(C)C)ccc1-c1cc(OCc2ccccc2)c(CO)c(C)n1 ; Cc1cc(C(C)(C)C)ccc1-c1cc(OCc2ccccc2)c(C=O)c(C)n1]

To a solution of [4-benzyloxy-6-(4-tert-butyl-2-methyl-phenyl)-2-methyl-3-pyridyl]methanol (OBn precursor of Compound 160, 580 mg, 0.21 mmol) in ethyl acetate (3 mL) was added 2-iodoxybenzoic acid (304 mg, 1.08 mmol) and the reaction was stirred at 80° C. for 2 h. The mixture was cooled to room temperature, diluted with ethyl acetate (10 mL), filtered through Celite® and washed with ethyl acetate (50 mL). The solution was washed with an aqueous saturated sodium bicarbonate solution (2×50 mL), dried over anhydrous sodium sulfate, filtered and evaporated under reduced pressure, affording 4-benzyloxy-6-(4-tert-butyl-2-methyl-phenyl)-2-methyl-pyridine-3-carbaldehyde (68 mg, 84%) as a light-yellow solid. ESI-MS m/z calc. 373.20, found 374.4 (M+1)⁺. ¹H NMR (400 MHz, DMSO-d₆) δ 10.60 (s, 1H), 7.54-7.49 (m, 2H), 7.46-7.39 (m, 2H), 7.39-7.34 (m, 2H), 7.33-7.28 (m, 3H), 5.41 (s, 2H), 2.67 (s, 3H), 2.29 (s, 3H), 1.31 (s, 9H) ppm.

### Step 2: 4-benzyloxy-6-(4-tert-butyl-2-methyl-phenyl)-2-methyl-3-[(E)-2-nitrovinyl]pyridine

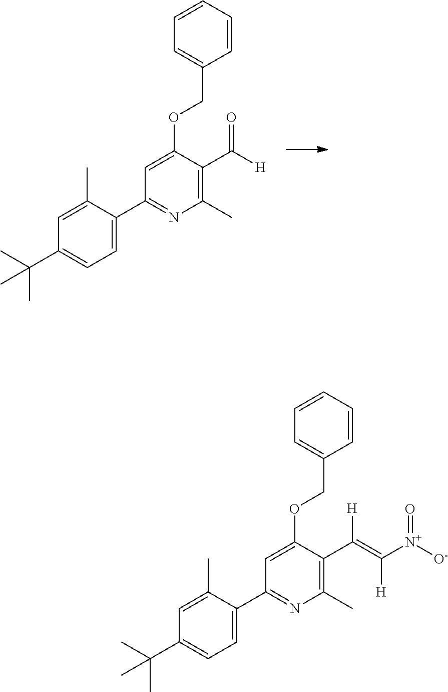 [SMILES: Cc1cc(C(C)(C)C)ccc1-c1cc(OCc2ccccc2)c(C=O)c(C)n1 ; Cc1cc(C(C)(C)C)ccc1-c1cc(OCc2ccccc2)c(/C=C/[N+](=O)[O-])c(C)n1]

A flask was charged with 4-benzyloxy-6-(4-tert-butyl-2-methyl-phenyl)-2-methyl-pyridine-3-carbaldehyde (780 mg, 1.97 mmol), nitromethane (18.03 g, 16 mL, 295.41 mol) and ammonium acetate (156 mg, 2.02 mmol) under nitrogen atmosphere at room temperature and was stirred for 90 min, It was then heated at 50° C. for 1 h followed by heating at 70° C. for 90 min. The mixture was partitioned between water (30 mL) and ethyl acetate (30 mL). The organic layer was washed with water (20 mL), brine (20 mL), dried over anhydrous sodium sulfate, filtered and evaporated under reduced pressure. Purification by silica gel chromatography using 0-10% ethyl acetate in heptane provided 4-benzyloxy-6-(4-tert-butyl-2-methyl-phenyl)-2-methyl-3-[(E)-2-nitrovinyl]pyridine (780 mg, 90%) as a yellow solid. ESI-MS m/z calc. 416.21, found 417.4 (M+1)⁺. ¹H NMR (400 MHz, CDCl₃) δ 8.33 (d, J=13.4 Hz, 1H), 8.01 (d, J=13.7 Hz, 1H), 7.47-7.38 (m, 5H), 7.33-7.28 (m, 3H), 6.94 (s, 1H), 5.29 (s, 2H), 2.78 (s, 3H), 2.31 (s, 3H), 1.34 (s, 9H) ppm.

### Step 3: 2-[4-benzyloxy-6-(4-tert-butyl-2-methyl-phenyl)-2-methyl-3-pyridyl]ethanamine

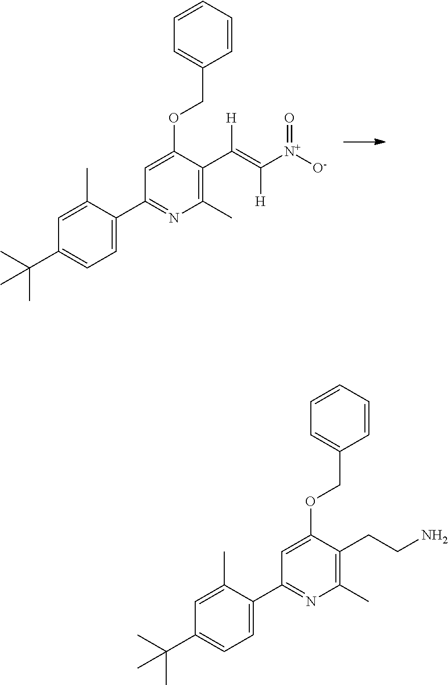 [SMILES: Cc1cc(C(C)(C)C)ccc1-c1cc(OCc2ccccc2)c(/C=C/[N+](=O)[O-])c(C)n1 ; Cc1cc(C(C)(C)C)ccc1-c1cc(OCc2ccccc2)c(CCN)c(C)n1]

To a solution at 0° C. of 4-benzyloxy-6-(4-tert-butyl-2-methyl-phenyl)-2-methyl-3-[(E)-2-nitrovinyl]pyridine (50 mg, 0.1154 mmol) in THF (1 mL) was added LiAlH₄ in THF (0.12 mL of 2 M, 0.24 mmol). The mixture was stirred at 0° C. for 5 min, then at room temperature for 1 h. The mixture was diluted with DCM (30 mL) and water (30 mL). The layers were separated and the organic layer was washed with a 1:1 solution of 5% aqueous citric acid/1 M HCl (30 mL), water (30 mL) and brine (30 mL), dried over anhydrous sodium sulfate, filtered and evaporated under reduced pressure. Purification by reverse-phase column chromatography (C₁₈) using 1 to 99% acetonitrile in water containing 5 mM HCl gave 2-[4-benzyloxy-6-(4-tert-butyl-2-methyl-phenyl)-2-methyl-3-pyridyl]ethanamine (20 mg, 44%) as a white solid. ESI-MS m/z calc. 388.25, found 389.2 (M+1)⁺. ¹H NMR (400 MHz, CD₃OD-d₄) δ 7.60-7.52 (m, 3H), 7.50-7.37 (m, 6H), 5.52 (s, 2H), 3.18 (s, 4H), 2.77 (s, 3H), 2.28 (s, 3H), 1.37 (s, 9H) ppm.

### Step 4: (2S)-2-amino-N-[2-[6-(4-tert-butyl-2-methyl-phenyl)-2-methyl-4-oxo-1H-pyridin-3-yl]ethyl]propanamide (160)

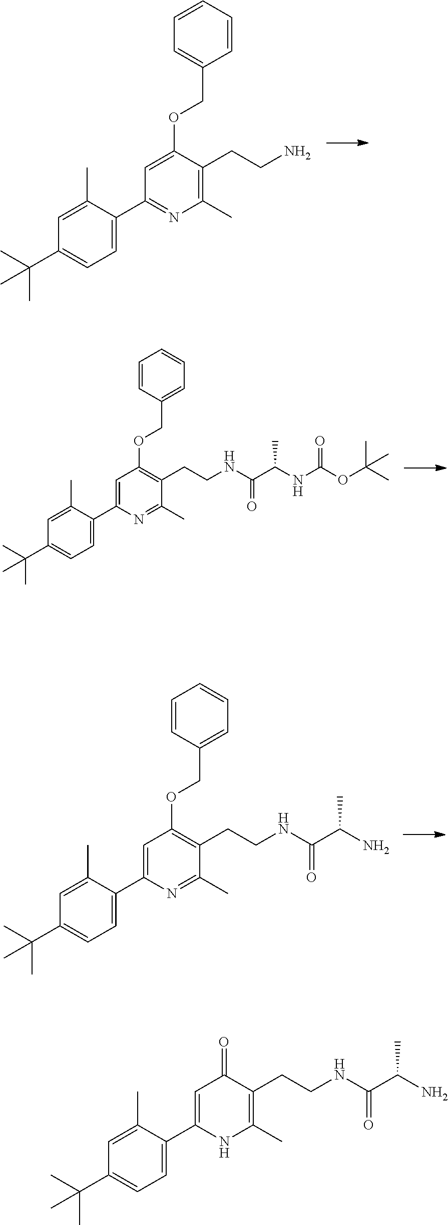

A solution of (2S)-2-(tert-butoxycarbonylamino)propanoic acid (15 mg, 0.079 mmol), DIPEA (20 mg, 0.15 mmol) and HATU (30 mg, 0.08 mmol) in DMF (1 mL) was stirred at room temperature for 10 min and it was added to 2-[4-benzyloxy-6-(4-tert-butyl-2-methyl-phenyl)-2-methyl-3-pyridyl]ethanamine (hydrochloride salt) (30 mg, 0.07 mmol). The mixture was stirred at room temperature for 2 h, filtered and purified by reverse phase HPLC (C₁₈) using 1 to 99% acetonitrile in water containing 5 mM HCl to give tert-butyl N-[(1S)-2-[2-[4-benzyloxy-6-(4-tert-butyl-2-methyl-phenyl)-2-methyl-3-pyridyl]ethylamino]-1-methyl-2-oxo-ethyl]carbamate (26.7 mg, 68%). ESI-MS m/z calc. 559.34, found 560.9 (M+1)⁺. It was treated with 1,4-dioxane solution of HCl (1 mL of 6 M, 6 mmol) and stirred at room temperature for 2 h. It was evaporated to give (2S)-2-amino-N-[2-[4-benzyloxy-6-(4-tert-butyl-2-methyl-phenyl)-2-methyl-3-pyridyl]ethyl]propanamide (hydrochloride salt) (23 mg, 66%). ESI-MS m/z calc. 459.29, found 460.7 (M+1)⁺. It was dissolved in methanol (2 mL) and Pd/C (3 mg of 60% w/w, 0.017 mmol) was added. The mixture was stirred and sparged with hydrogen gas for 30 min. The mixture was filtered and purified by reverse phase HPLC (C₁₈) using 1 to 99% acetonitrile in water containing 5 mM HCl to give (2S)-2-amino-N-[2-[6-(4-tert-butyl-2-methyl-phenyl)-2-methyl-4-oxo-1H-pyridin-3-yl]ethyl]propanamide (hydrochloride salt) (160, 12.3 mg, 42%). ESI-MS m/z calc. 369.24, found 370.5 (M+1)⁺. ¹H NMR (400 MHz, DMSO-d₆) δ 13.98 (s, 1H), 8.73 (t, J=6.0 Hz, 1H), 8.1-8.3 (m, 3H), 7.46 (s, 1H), 7.43 (dd, J=8.0, 2.0 Hz, 1H), 7.34 (d, J=8.0 Hz, 1H), 7.25 (s, 1H), 3.79 (t, J 6.2 Hz, 1H), 3.2-3.33 (m, 2H), 2.81 (t, J=7.3 Hz, 2H), 2.63 (s, 3H), 2.28 (s, 3H), 1.33 (s, 9H), 1.31 (s, 3H) ppm.

### Example 19

### 6-(4-(tert-butyl)-5-chloro-2-methylphenyl)-4-oxo-1,4-dihydropyridine-3-carboxamide (161)

### Step 1: methyl 6-(4-tert-butyl-5-chloro-2-methyl-phenyl)-4-methoxy-pyridine-3-carboxylate (161)

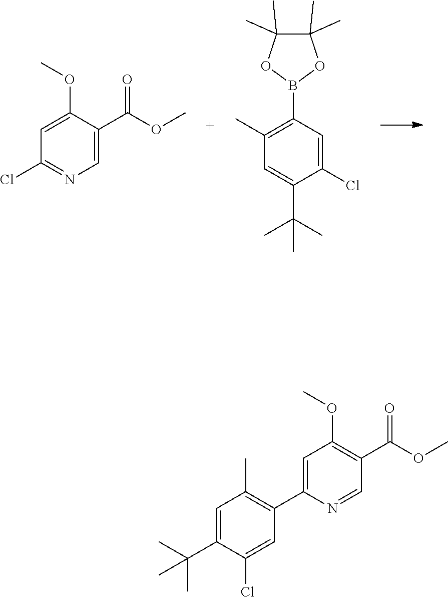 [SMILES: COC(=O)c1cnc(Cl)cc1OC ; Cc1cc(C(C)(C)C)c(Cl)cc1B1OC(C)(C)C(C)(C)O1 ; COC(=O)c1cnc(-c2cc(Cl)c(C(C)(C)C)cc2C)cc1OC]

A 1,4-dioxane (9 mL) solution of methyl 6-chloro-4-methoxy-pyridine-3-carboxylate (80 mg, 0.39 mmol), 2-(4-tert-butyl-5-chloro-2-methyl-phenyl)-4,4,5,5-tetramethyl-1,3,2-dioxaborolane (Intermediate B-2, 100 mg, 0.32 mmol), Pd(dppf)Cl₂·DCM (10.6 mg, 0.016 mmol) and potassium phosphate (1 mL of 1 M, 1 mmol) was sparged with nitrogen for 5 min and then stirred at room temperature for 1 h. The mixture was diluted with ethyl acetate and washed with water and brine. The organic phase was dried over sodium sulfate, filtered and concentrated in vacuo. Purification by silica gel chromatography using 0 to 30% ethyl acetate in hexanes yielded methyl 6-(4-tert-butyl-5-chloro-2-methyl-phenyl)-4-methoxy-pyridine-3-carboxylate (60 mg, 53%). ESI-MS m/z calc. 347.13, found 348.2 (M+1)⁺.

### Step 2: 6-(4-tert-butyl-5-chloro-2-methyl-phenyl)-4-oxo-1H-pyridine-3-carboxamide (161)

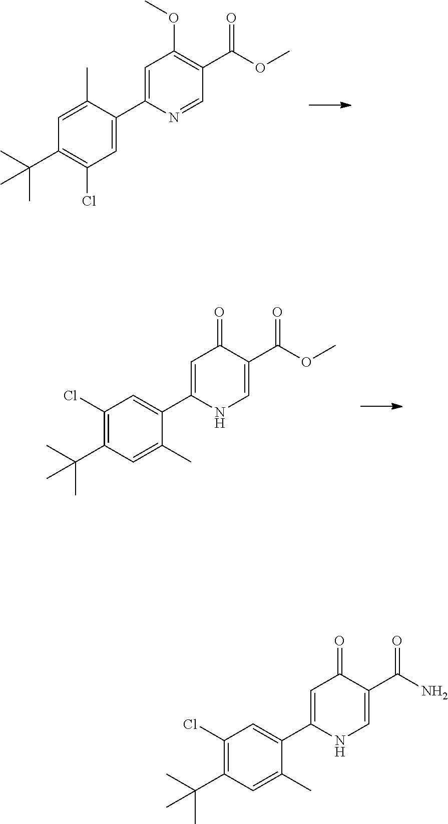

A solution of methyl 6-(4-tert-butyl-5-chloro-2-methyl-phenyl)-4-methoxy-pyridine-3-carboxylate (12 mg, 0.03 mmol) in DCM (250 μL) was cooled in a dry ice-acetone bath and boron tribromide in DCM (100 μL of 1 M, 0.1 mmol) was added under an atmosphere of nitrogen. After 5 min the bath was removed, and the mixture was stirred for 15 min. The mixture was slowly quenched with methanol. The solvent was evaporated, and the crude material was purified via silica gel column chromatography using 0 to 10% methanol in DCM to obtain the pyridone ester intermediate, which was treated with NH₃ (1 mL of 7 M, 7 mmol) (methanol solution) and stirred at 60° C. for 16 h. The reaction was concentrated in vacuo and purified by reverse phase HPLC (C₁₈) using 1 to 100% acetonitrile in water containing 5 mM HCl to give 6-(4-tert-butyl-5-chloro-2-methyl-phenyl)-4-oxo-1H-pyridine-3-carboxamide (161, 1.1 mg, 10%). ESI-MS m/z calc. 318.11, found 319.2 (M+1)⁺. ¹H NMR (400 MHz, CD₃OD) δ 8.57 (s, 1H), 7.47 (s, 1H), 7.39 (s, 1H), 6.53 (s, 1H), 2.31 (s, 3H), 1.51 (s, 9H) ppm.

### Example 20

### 6-[4-tert-butyl-2-(2-hydroxyethoxy)phenyl]-2,3-dimethyl-1H-pyridin-4-one (162)

### Step 1: 6-[4-tert-butyl-2-(2-hydroxyethoxy)phenyl]-2,3-dimethyl-1H-pyridin-4-one (162)

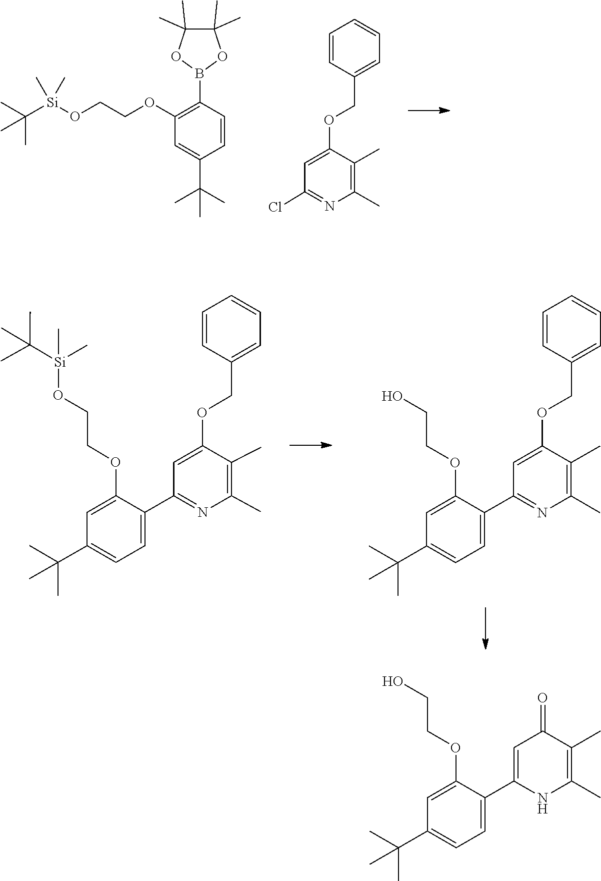 [SMILES: CC(C)(C)c1ccc(B2OC(C)(C)C(C)(C)O2)c(OCCO[Si](C)(C)C(C)(C)C)c1 ; Cc1nc(Cl)cc(OCc2ccccc2)c1C ; Cc1nc(-c2ccc(C(C)(C)C)cc2OCCO[Si](C)(C)C(C)(C)C)cc(OCc2ccccc2)c1C ; Cc1nc(-c2ccc(C(C)(C)C)cc2OCCO)cc(OCc2ccccc2)c1C ; Cc1[nH]c(-c2ccc(C(C)(C)C)cc2OCCO)cc(=O)c1C]

To crude tert-butyl-[2-[5-tert-butyl-2-(4,4,5,5-tetramethyl-1,3,2-dioxaborolan-2-yl)phenoxy]ethoxy]-dimethyl-silane was added 4-benzyloxy-6-chloro-2,3-dimethyl-pyridine (106 mg, 0.43 mmol), Pd(PPh₃)₄(39 mg, 0.03 mmol), potassium carbonate (134 mg, 0.97 mmol), 1,4-dioxane (5 mL), and water (1 mL). The reaction was purged with nitrogen for 30 seconds, capped and stirred at 95° C. for 16 h. After cooling to room temperature, the crude mixture was extracted with DCM (3×20 mL). The combined organic layer was washed with brine (˜10 mL), dried over sodium sulfate, filtered and concentrated in vacuo to afford 4-(benzyloxy)-6-(4-(tert-butyl)-2-(2-((tert-butyldimethylsilyl)oxy)ethoxy)phenyl)-2,3-dimethylpyridine.

The crude 4-(benzyloxy)-6-(4-(tert-butyl)-2-(2-((tert-butyldimethylsilyl)oxy)ethoxy)phenyl)-2,3-dimethylpyridine was taken up in DCM (5 mL) and TFA (250 μL, 3.24 mmol), and the mixture stirred at room temperature for 4 h. The volatiles were removed in vacuo and to it was added palladium on carbon (38 mg of 10% w/w, 0.036 mmol) and ethyl acetate (10 mL). The mixture was sparged with hydrogen for 5 min and the mixture stirred at room temperature under hydrogen atmosphere for 12 h. The solids were filtered over Celite® and the resulting solution concentrated in vacuo. HPLC purification (C₁₈) using 1 to 99% acetonitrile in water containing 5 mM HCl gave 6-[4-tert-butyl-2-(2-hydroxyethoxy)phenyl]-2,3-dimethyl-1H-pyridin-4-one (162, 2 mg, 2%). ESI-MS m/z calc. 315.18, found 316.4 (M+1)⁺.

### Example 21

### 6-[4-tert-butyl-2-(2-methoxyethoxy)phenyl]-2,3-dimethyl-1H-pyridin-4-one (163)

### Step 1: 6-[4-tert-butyl-2-(2-methoxyethoxy)phenyl]-2,3-dimethyl-1H-pyridin-4-one (163)

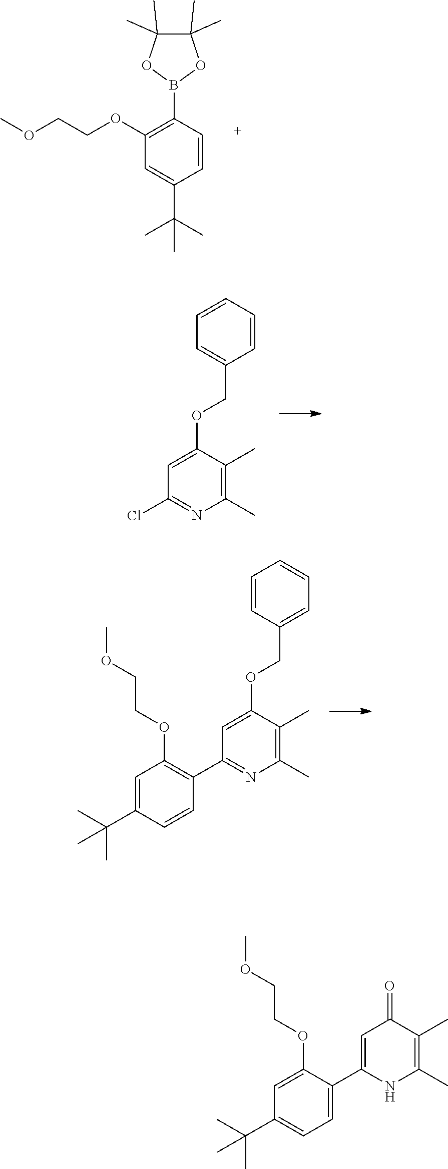

A via was charged with 4-benzyloxy-6-chloro-2,3-dimethyl-pyridine (Intermediate A-10, 38 mg, 1 mmol), crude 2-[4-tert-butyl-2-(2-methoxyethoxy)phenyl]-4,4,5,5-tetramethyl-1,3,2-dioxaborolane (Intermediate B-17), XPhos Pd G2 (12 mg, 0.02 mmol), XPhos (12 mg, 0.03 mmol), potassium carbonate (75 mg, 0.54 mmol), EtOH (4 mL) and water (1 mL). The reaction was purged with nitrogen for 30 seconds, capped and the mixture stirred at 95° C. for 18 h. After cooling to room temperature, the volatiles were removed in vacuo, and ethyl acetate was added. The organic layer was washed with water (10 mL) and brine (10 mL), dried over sodium sulfate, filtered and concentrated in vacuo to give 4-benzyloxy-6-[4-tert-butyl-2-(2-methoxyethoxy)phenyl]-2,3-dimethyl-pyridine. To it was added Pd/C (30 mg of 10% w/w, 0.03 mmol) and ethyl acetate (10 mL). The mixture was sparged with hydrogen gas for 1 min, and the mixture stirred under hydrogen atmosphere for 16 h. The reaction was filtered over Celite® and concentrated in vacuo. Purification by HPLC (C₁₈) using 1 to 99% acetonitrile in water containing 5 mM HCl provided 6-[4-tert-butyl-2-(2-methoxyethoxy)phenyl]-2,3-dimethyl-1H-pyridin-4-one (163, 6 mg, 12%). ESI-MS m/z calc. 329.2, found 330.4 (M+1)⁺.

### Example 22

### 2-[4-tert-butyl-2-(2-hydroxyethoxy)phenyl]-6-methyl-1H-pyridin-4-one (164)

### Step 1: 2-[4-tert-butyl-2-(2-hydroxyethoxy)phenyl]-6-methyl-1H-pyridin-4-one (164)

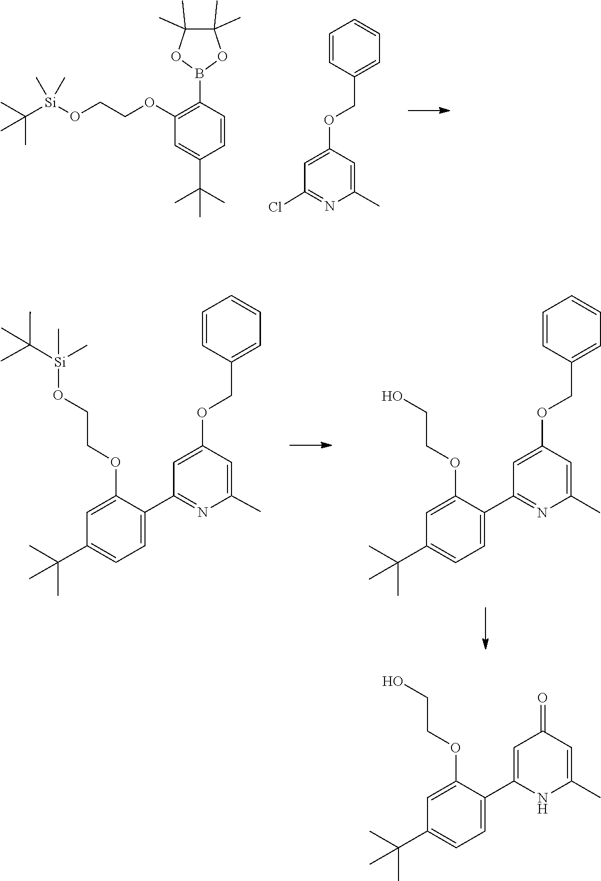 [SMILES: CC(C)(C)c1ccc(B2OC(C)(C)C(C)(C)O2)c(OCCO[Si](C)(C)C(C)(C)C)c1 ; Cc1cc(OCc2ccccc2)cc(Cl)n1 ; Cc1cc(OCc2ccccc2)cc(-c2ccc(C(C)(C)C)cc2OCCO[Si](C)(C)C(C)(C)C)n1 ; Cc1cc(OCc2ccccc2)cc(-c2ccc(C(C)(C)C)cc2OCCO)n1 ; Cc1cc(=O)cc(-c2ccc(C(C)(C)C)cc2OCCO)[nH]1]

To the crude tert-butyl-[2-[5-tert-butyl-2-(4,4,5,5-tetramethyl-1,3,2-dioxaborolan-2-yl)phenoxy]ethoxy]-dimethyl-silane (Intermediate B-9) in a 20 mL vial was added 4-benzyloxy-2-chloro-6-methyl-pyridine (Intermediate A-12, 95 mg, 0.41 mmol), Pd(PPh₃)₄ (35 mg, 0.03 mmol), potassium carbonate (185 mg, 1.339 mmol), 1,4-dioxane (4 mL) and water (1 mL). The mixture was purged with nitrogen for 30 seconds, capped and stirred at 95° C. for 16 h. After cooling to room temperature, the crude mixture was extracted with DCM (3×20 mL). The combined organic layer was washed with brine (˜10 mL), dried over sodium sulfate, filtered and concentrated in vacuo to afford crude 4-(benzyloxy)-2-(4-(tert-butyl)-2-(2-((tert-butyldimethylsilyl)oxy)ethoxy)phenyl)-6-methylpyridine.

The crude 4-(benzyloxy)-2-(4-(tert-butyl)-2-(2-((tert-butyldimethylsilyl)oxy)ethoxy)phenyl)-6-methylpyridine was taken up in DCM (5 mL) and TFA and stirred at room temperature for 4 h. The volatiles were removed in vacuo affording 2-[2-(4-benzyloxy-6-methyl-2-pyridyl)-5-tert-butyl-phenoxy]ethanol. It was added to a flask charged with palladium on carbon (55 mg of 10% w/w, 0.05 mmol) as solution in ethyl acetate (10 mL). The mixture was sparged with hydrogen for 5 min and the mixture stirred at room temperature under hydrogen atmosphere for 12 h. It was filtered over Celite® and concentrated in vacuo. Purification by reverse phase HPLC (C₁₈) using 1 to 99% acetonitrile in water containing 5 mM HCl provided 2-[4-tert-butyl-2-(2-hydroxyethoxy)phenyl]-6-methyl-1H-pyridin-4-one (164, 2 mg, 2%). ESI-MS m/z calc. 301.17, found 302.4 (M+1)⁺.

### Example 23

### 6-[5-chloro-2-methyl-4-(2,2,2-trifluoro-1,1-dimethyl-ethyl)phenyl]-2-methyl-3-methylsulfonyl-1H-pyridin-4-one (165) and 2-methyl-3-methylsulfonyl-6-[2-methyl-4-(2,2,2-trifluoro-1,1-dimethyl-ethyl)phenyl]-1H-pyridin-4-one (166)

### Step 1: 4-benzyloxy-6-[5-chloro-2-methyl-4-(2,2,2-trifluoro-1,1-dimethyl-ethyl)phenyl]-2-methyl-3-methylsulfanyl-pyridine

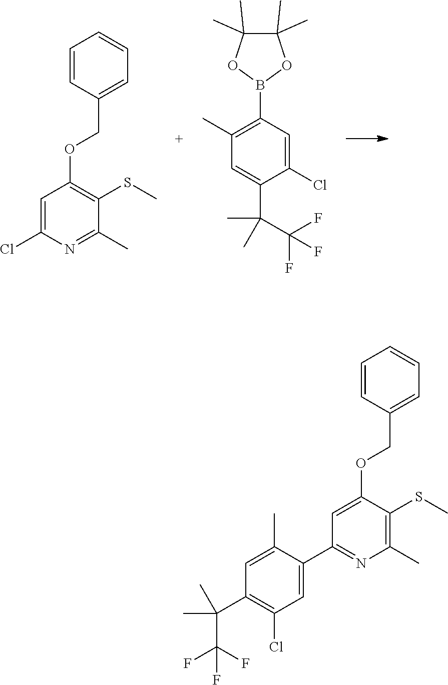 [SMILES: CSc1c(OCc2ccccc2)cc(Cl)nc1C ; Cc1cc(C(C)(C)C(F)(F)F)c(Cl)cc1B1OC(C)(C)C(C)(C)O1 ; CSc1c(OCc2ccccc2)cc(-c2cc(Cl)c(C(C)(C)C(F)(F)F)cc2C)nc1C]

4-Benzyloxy-6-chloro-2-methyl-3-methylsulfanyl-pyridine (Intermediate A-5, Step 4, 135 mg, 0.46 mmol), 2-[5-chloro-2-methyl-4-(2,2,2-trifluoro-1,1-dimethyl-ethyl)phenyl]-4,4,5,5-tetramethyl-1,3,2-dioxaborolane (Intermediate B-4, 180 mg, 0.5 mmol) and aqueous potassium phosphate (1.5 mL of 1 M, 1.5 mmol) were combined in 1,4-dioxane (3 mL) and purged with nitrogen for 5 min. PdCl₂(dtbpf) (32 mg, 0.05 mmol) was added and the reaction was purged with nitrogen for an additional 5 min. The mixture was stirred at room temperature for 16 h. The reaction was partitioned between ethyl acetate and water. The organic layer was separated, washed with brine, dried over sodium sulfate, filtered and evaporated. The crude material was purified via silica gel column chromatography using 0 to 8% ethyl acetate in hexanes to give 4-benzyloxy-6-[5-chloro-2-methyl-4-(2,2,2-trifluoro-1,1-dimethyl-ethyl)phenyl]-2-methyl-3-methylsulfanyl-pyridine (107.7 mg, 48%). ESI-MS m/z calc. 479.13, found 480.3 (M+1)⁺. ¹H NMR (400 MHz, CDCl₃) δ 7.48-7.40 (m, 4H), 7.40-7.33 (m, 3H), 6.77 (s, 1H), 5.24 (s, 2H), 2.77 (s, 3H), 2.40 (s, 3H), 2.22 (s, 3H), 1.76 (s, 6H) ppm. ¹⁹F NMR (376 MHz, CDCl₃) δ −73.84 ppm.

### Step 2: 6-[5-chloro-2-methyl-4-(2,2,2-trifluoro-1,1-dimethyl-ethyl)phenyl]-2-methyl-3-methylsulfonyl-1H-pyridin-4-one (165) and 2-methyl-3-methylsulfonyl-6-[2-methyl-4-(2,2,2-trifluoro-1,1-dimethyl-ethyl)phenyl]-1H-pyridin-4-one (166)

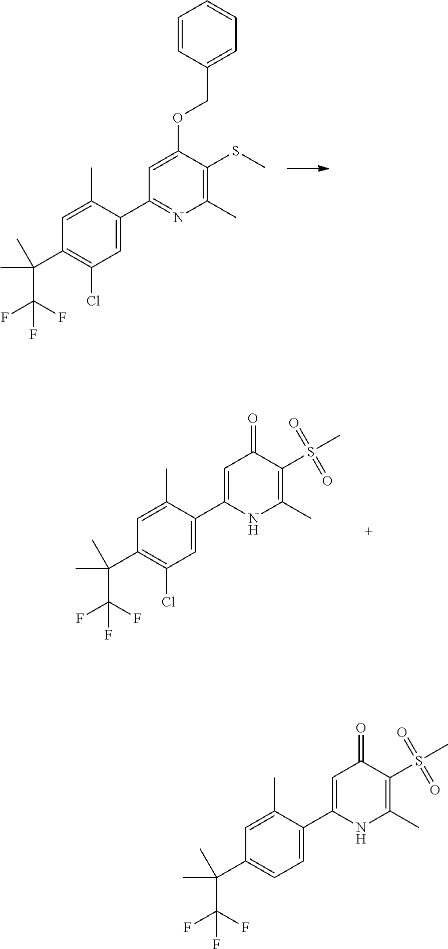 [SMILES: CSc1c(OCc2ccccc2)cc(-c2cc(Cl)c(C(C)(C)C(F)(F)F)cc2C)nc1C ; Cc1cc(C(C)(C)C(F)(F)F)c(Cl)cc1-c1cc(=O)c(S(C)(=O)=O)c(C)[nH]1 ; Cc1cc(C(C)(C)C(F)(F)F)ccc1-c1cc(=O)c(S(C)(=O)=O)c(C)[nH]1]

To a solution of 4-benzyloxy-6-[5-chloro-2-methyl-4-(2,2,2-trifluoro-1,1-dimethyl-ethyl)phenyl]-2-methyl-3-methylsulfanyl-pyridine (100 mg, 0.21 mmol) in DCM (4 mL) was added m-CPBA (100 mg, 0.45 mmol) in one portion at 0° C. and the reaction stirred for 2 h at this temperature. The mixture was quenched with saturated sodium bicarbonate solution (2 mL). The mixture was extracted with DCM (3×5 mL). The combined organic phases were dried over sodium sulfate, filtered and concentrated in vacuo. The residue was dissolved in methanol (5 mL) and 10% Pd/C (20 mg, 0.19 mmol) was added. The mixture was stirred at room temperature under an atmosphere of hydrogen. The mixture was concentrated under reduced pressure, filtered, and purified by reverse phase chromatography (C₁₈) using 20 to 60% acetonitrile in water containing 5 mM HCl to give 6-[5-chloro-2-methyl-4-(2,2,2-trifluoro-1,1-dimethyl-ethyl)phenyl]-2-methyl-3-methylsulfonyl-1H-pyridin-4-one (165, 45.5 mg, 52%). ESI-MS m/z calc. 421.07, found 422.2 (M+1)⁺. ¹H NMR (400 MHz, DMSO-d₆) δ 11.86 (s, 1H), 7.65 (s, 1H), 7.53 (s, 1H), 6.29 (s, 1H), 3.30 (s, 3H), 2.62 (s, 3H), 2.29 (s, 3H), 1.78 (s, 6H) ppm. ¹⁹F NMR (376 MHz, DMSO-d₆) δ −70.72 ppm; and 2-methyl-3-methylsulfonyl-6-[2-methyl-4-(2,2,2-trifluoro-1,1-dimethyl-ethyl)phenyl]-1H-pyridin-4-one (166, 3.8 mg, 5%). ESI-MS m/z calc. 387.11, found 388.2 (M+1)⁺. ¹H NMR (400 MHz, DMSO-d₆) δ 11.88 (s, 1H), 7.56 (d, J=2.0 Hz, 1H), 7.53-7.48 (m, 1H), 7.39 (d, J=8.1 Hz, 1H), 6.23 (s, 1H), 3.30 (s, 3H), 2.63 (s, 3H), 2.32 (s, 3H), 1.59 (s, 6H) ppm. ¹⁹F NMR (376 MHz, DMSO-d₆) δ −74.67 ppm.

### Example 24

### 6-[4-tert-butyl-2-methyl-5-(trifluoromethyl)phenyl]-2-methyl-3-methylsulfonyl-1H-pyridin-4-one (167)

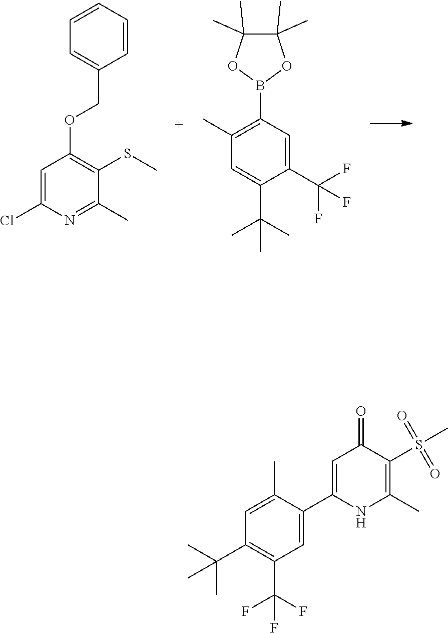 [SMILES: CSc1c(OCc2ccccc2)cc(Cl)nc1C ; Cc1cc(C(C)(C)C)c(C(F)(F)F)cc1B1OC(C)(C)C(C)(C)O1 ; Cc1cc(C(C)(C)C)c(C(F)(F)F)cc1-c1cc(=O)c(S(C)(=O)=O)c(C)[nH]1]

6-[4-tert-Butyl-2-methyl-5-(trifluoromethyl)phenyl]-2-methyl-3-methylsulfonyl-1H-pyridin-4-one (167, 13.8 mg, 35%) was prepared using procedure analogous to Example 23 (Step 1 and Step 2), using 2-[4-tert-butyl-2-methyl-5-(trifluoromethyl)phenyl]-4,4,5,5-tetramethyl-1,3,2-dioxaborolane (Intermediate B-15). ESI-MS m/z calc. 401.13, found 402.3 (M+1)⁺. ¹H NMR (400 MHz, DMSO-d₆) δ 11.85 (s, 1H), 7.73 (s, 1H), 7.71 (s, 1H), 6.29 (d, J=1.8 Hz, 1H), 3.30 (s, 3H), 2.62 (s, 3H), 2.36 (s, 3H), 1.45 (s, 9H) ppm. ¹⁹F NMR (376 MHz, DMSO-d₆) δ −51.53 ppm.

### Example 25

### 2-(4-tert-butyl-5-chloro-2-methyl-phenyl)-5,6-dimethyl-3-(4-methyloxazol-2-yl)-1H-pyridin-4-one (168)

### Step 1: 3-bromo-2-(4-tert-butyl-5-chloro-2-methyl-phenyl)-5,6-dimethyl-1H-pyridin-4-one

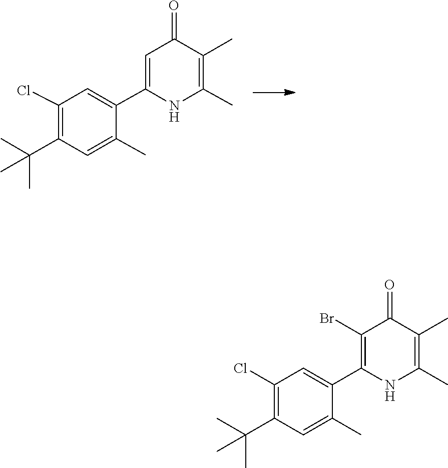 [SMILES: Cc1cc(C(C)(C)C)c(Cl)cc1-c1cc(=O)c(C)c(C)[nH]1 ; Cc1cc(C(C)(C)C)c(Cl)cc1-c1[nH]c(C)c(C)c(=O)c1Br]

6-(4-tert-butyl-5-chloro-2-methyl-phenyl)-2,3-dimethyl-1H-pyridin-4-one (465 mg, 1.53 mmol) was dissolved in DCM (9.3 mL). To it was added NBS (300 mg, 1.69 mmol) and stirred overnight at room temperature. Volatiles were removed at reduced pressure and purification via silica gel column chromatography using 0 to 100% EtOAc in hexanes gave 3-bromo-2-(4-tert-butyl-5-chloro-2-methyl-phenyl)-5,6-dimethyl-1H-pyridin-4-one (480 mg, 82%) as a white solid. ESI-MS m/z calc. 381.05, found 382.2 (M+1)⁺.

### Step 2: 4-benzyloxy-3-bromo-2-(4-tert-butyl-5-chloro-2-methyl-phenyl)-5,6-dimethyl-pyridine

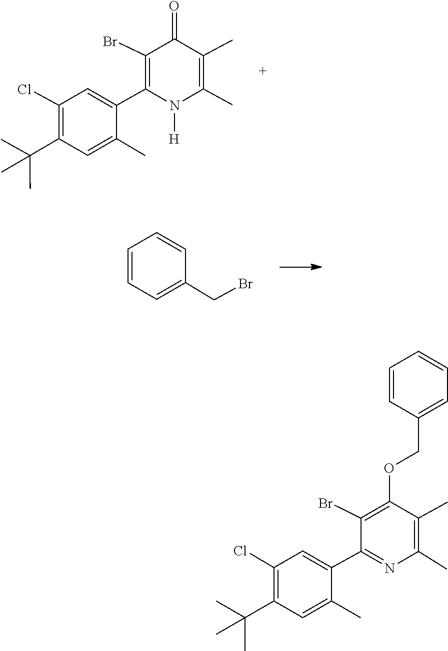 [SMILES: Cc1cc(C(C)(C)C)c(Cl)cc1-c1[nH]c(C)c(C)c(=O)c1Br ; BrCc1ccccc1 ; Cc1cc(C(C)(C)C)c(Cl)cc1-c1nc(C)c(C)c(OCc2ccccc2)c1Br]

3-bromo-2-(4-tert-butyl-5-chloro-2-methyl-phenyl)-5,6-dimethyl-1H-pyridin-4-one (130 mg, 0.34 mmol) was dissolved in THF (10 mL). To the stirred solution was added bromomethylbenzene (581 mg, 404.0 μL, 3.4 mmol) and potassium carbonate (470 mg, 3.4 mmol) and heated to 45° C. for 2 hours. Volatiles were removed at reduced pressure and the crude residue was purified vis silica gel column chromatography using 0 to 100% DCM in hexanes to obtain 4-benzyloxy-3-bromo-2-(4-tert-butyl-5-chloro-2-methyl-phenyl)-5,6-dimethyl-pyridine (155 mg, 97%) as a clear colorless solid ESI-MS m/z calc. 471.09, found 472.4 (M+1)⁺.

### Step 3: 2-(4-tert-butyl-5-chloro-2-methyl-phenyl)-5,6-dimethyl-3-(4-methyloxazol-2-yl)-1H-pyridin-4-one (168)

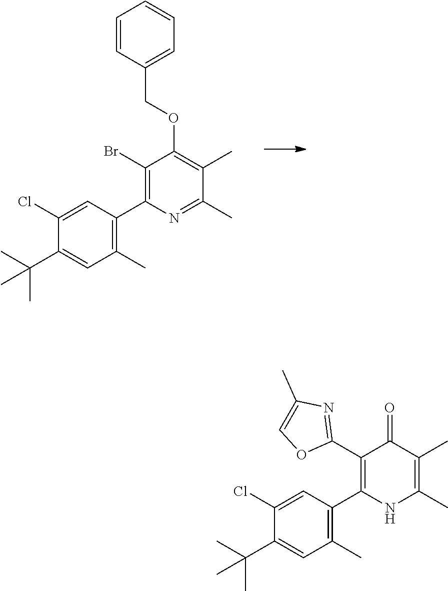 [SMILES: Cc1cc(C(C)(C)C)c(Cl)cc1-c1nc(C)c(C)c(OCc2ccccc2)c1Br ; Cc1coc(-c2c(-c3cc(Cl)c(C(C)(C)C)cc3C)[nH]c(C)c(C)c2=O)n1]

A microwave vial was charged with 4-benzyloxy-3-bromo-2-(4-tert-butyl-5-chloro-2-methyl-phenyl)-5,6-dimethyl-pyridine (32 mg, 0.07 mmol), tributyl-(4-methyloxazol-2-yl)stannane (76 mg, 0.20 mmol), PdCl₂(PPh₃)₂(10 mg, 0.013 mmol) and copper (I) iodide (13 mg, 0.07 mmol). Toluene (640 μL) was added and the reaction mixture was degassed under nitrogen. The tube was sealed and stirred at 110° C. for 20 hours. Purification using high pressure reverse phase chromatography using 1 to 100% ACN in water containing 5 mM hydrochloric acid gave 2-[4-benzyloxy-2-(4-tert-butyl-5-chloro-2-methyl-phenyl)-5,6-dimethyl-3-pyridyl]-4-methyl-oxazole as a yellow oil. ESI-MS m/z calc. 474.20, found 475.77 (M+1)⁺. It was dissolved in ethanol (4 mL) and to it was added Pd/C (10 mg of 10% w/w, 0.009 mmol). The reaction mixture was stirred under an atmosphere of hydrogen for 10 minutes. The reaction mixture was filtered and purified via high pressure reverse phase chromatography using 1 to 100% ACN in water containing 5 mM hydrochloric acid to obtain 2-(4-tert-butyl-5-chloro-2-methyl-phenyl)-5,6-dimethyl-3-(4-methyloxazol-2-yl)-1H-pyridin-4-one (168, 1.7 mg, 6%) as a white solid ESI-MS m/z calc. 384.16, found 385.41 (M+1)⁺. ¹H NMR (400 MHz, CD₃OD) δ 7.54 (d, J=1.5 Hz, 1H), 7.43 (s, 1H), 7.33 (s, 1H), 2.53 (s, 3H), 2.25 (s, 3H), 2.15 (d, J=1.2 Hz, 3H), 2.10 (s, 3H), 1.50 (s, 9H).

The following compounds were synthesized using the route shown in Example-25 (Step 1 and Step 2), using appropriate heterocyclic stannanes. Stille coupling followed by deprotection provided the following compounds.

TABLE 4

| | | LC/MS | |
|---|---|---|---|
| | | (m/z calc.) | |
| Cmpd. | | Found | NMR (shifts |
| No. | Compound Name | [M + H]⁺ | in ppm) |
| 169 | 2-(4-tert-butyl-5-chloro-2- | 384.17 | |
| | methyl-phenyl)-5,6-dimethyl- | 385.6 | |
| | 3-(2-methyltriazol-4-yl)-1H- | | |
| | pyridin-4-one | | |

### Example 26

### 2-(4-tert-butyl-5-chloro-2-methyl-phenyl)-3-[1-(methoxymethyl)pyrazol-4-yl]-5,6-dimethyl-1H-pyridin-4-one (170)

### Step 1: 2-(4-tert-butyl-5-chloro-2-methyl-phenyl)-3-[1-(methoxymethyl)pyrazol-4-yl]-5,6-dimethyl-1H-pyridin-4-one (170)

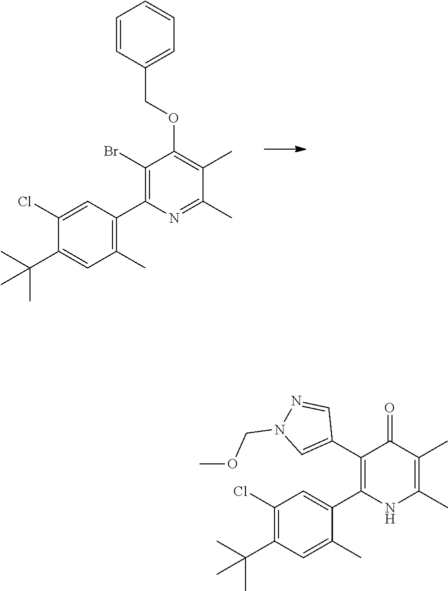

A microwave vial was charged with 4-benzyloxy-3-bromo-2-(4-tert-butyl-5-chloro-2-methyl-phenyl)-5,6-dimethyl-pyridine (25 mg, 0.05 mmol), [1-(methoxymethyl)pyrazol-4-yl]boronic acid (18 mg, 0.12 mmol), PdCl2(dtbpf) (10 mg, 0.015 mmol), and potassium phosphate (46 mg, 0.22 mmol). Dioxane (1 mL) and water (290 μL) were added and the reaction mixture was degassed for 5 minutes under nitrogen. The tube was sealed and stirred at 45° C. for 30 minutes. The resulting mixture was diluted with ethyl acetate, washed with a saturated aqueous solution of ammonium chloride and brine. The organic layer was dried over anhydrous magnesium sulfate, filtered, and concentrated under reduced pressure. Purification by silica gel chromatography using 0 to 20% of methanol in DCM gave 4-benzyloxy-2-(4-tert-butyl-5-chloro-2-methyl-phenyl)-3-[1-(methoxymethyl)pyrazol-4-yl]-5,6-dimethyl-pyridine. ESI-MS m/z calc. 503.23, found 504.5 (M+1)⁺. It was dissolved in ethanol (4 mL) and Pd/C (10 mg of 10% w/w, 0.009 mmol) was added. The reaction mixture was stirred under an atmosphere of hydrogen for 10 minutes. The reaction mixture was filtered and purified via high pressure reverse phase chromatography using 1 to 100% ACN in water (5 mM hydrochloric acid) to obtain 2-(4-tert-butyl-5-chloro-2-methyl-phenyl)-3-[1-(methoxymethyl)pyrazol-4-yl]-5,6-dimethyl-1H-pyridin-4-one (170, 3 mg, 13%) clear colorless solid ESI-MS m/z calc. 413.187, found 414.5 (M+1)⁺. ¹H NMR (400 MHz, CD₃OD) δ 7.71 (s, 1H), 7.38 (s, 1H), 7.32 (s, 1H), 7.15 (s, 1H), 5.26 (s, 2H), 3.10 (s, 3H), 2.38 (s, 3H), 2.12 (s, 3H), 2.02 (s, 3H), 1.48 (s, 9H).

The following compounds were synthesized using the route shown in Example 26 (Step 1 and Step 2), using appropriate heterocyclic boronates. Suzuki coupling followed by deprotection provided the following compounds.

TABLE 5

| | | LC/MS | |
|---|---|---|---|
| | | (m/z calc.); | |
| Cmpd. | | Found | NMR (shifts |
| No. | Compound Name | [M + H]⁺ | in ppm) |
| 171 | 2-(4-tert-butyl-5-chloro-2- | 419.16 | |
| | methyl-phenyl)-3-[1- | 420.36 | |
| | (difluoromethyl)pyrazol-3- | | |
| | yl]-5,6-dimethyl-1H- | | |
| | pyridin-4-one | | |

### Example 27

### 3-[6-(4-tert-butyl-2-methyl-phenyl)-2-methyl-4-oxo-1H-pyridin-3-yl]pyridine-2-carbonitrile (172)

### Step 1: ethyl 4-benzyloxy-6-(4-tert-butyl-2-methyl-phenyl)-2-methyl-pyridine-3-carboxylate

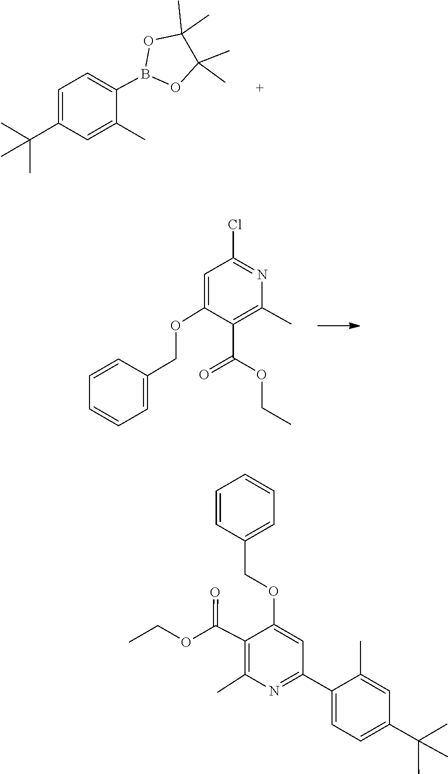 [SMILES: Cc1cc(C(C)(C)C)ccc1B1OC(C)(C)C(C)(C)O1 ; CCOC(=O)c1c(OCc2ccccc2)cc(Cl)nc1C ; CCOC(=O)c1c(OCc2ccccc2)cc(-c2ccc(C(C)(C)C)cc2C)nc1C]

In a sealed tube, a mixture of 2-(4-tert-butyl-2-methyl-phenyl)-4,4,5,5-tetramethyl-1,3,2-dioxaborolane (524 mg, 1.91 mmol), ethyl 4-benzyloxy-6-chloro-2-methyl-pyridine-3-carboxylate (630 mg, 1.88 mmol), aqueous sodium bicarbonate (7.9 mL of 1.1 M, 8.69 mmol) and dioxane (10 mL) was bubbled with nitrogen for 5 minutes. Pd(dppf)Cl₂DCM (160 mg, 0.2 mmol) was added and the reaction mixture was purged with nitrogen for 10 minutes. The tube was sealed and the reaction mixture was stirred at 110° C. for 16 h. The reaction mixture was diluted with ethyl acetate (50 mL). The organic layer was washed with water (2×30 mL) and brine (30 mL), dried over sodium sulfate, filtered and concentrated. Purification by silica gel column chromatography using 0 to 20% ethyl acetate in heptane provided ethyl 4-benzyloxy-6-(4-tert-butyl-2-methyl-phenyl)-2-methyl-pyridine-3-carboxylate (702 mg, 89%) as a colorless oil. ESI-MS m/z calc. 417.23, found 418.4 (M+1)⁺. ¹H NMR (400 MHz, CDCl₃) δ 7.41-7.31 (m, 5H), 7.30-7.24 (m, 3H), 6.82 (s, 1H), 5.18 (s, 2H), 4.44 (q, J=7.1 Hz, 2H), 2.58 (s, 3H), 2.26 (s, 3H), 1.37 (t, J=7.2 Hz, 3H), 1.33 (s, 9H).

### Step 2: 4-benzyloxy-6-(4-tert-butyl-2-methyl-phenyl)-2-methyl-pyridine-3-carboxylic acid

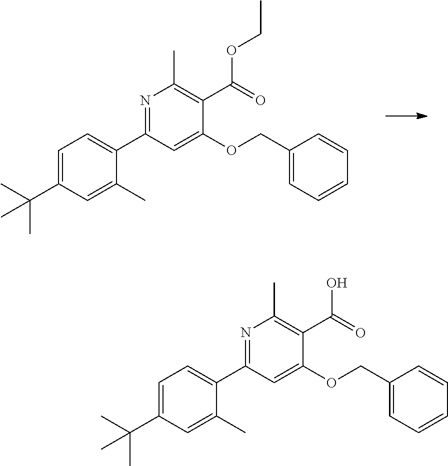 [SMILES: CCOC(=O)c1c(OCc2ccccc2)cc(-c2ccc(C(C)(C)C)cc2C)nc1C ; Cc1cc(C(C)(C)C)ccc1-c1cc(OCc2ccccc2)c(C(=O)O)c(C)n1]

To a suspension of ethyl 4-benzyloxy-6-(4-tert-butyl-2-methyl-phenyl)-2-methyl-pyridine-3-carboxylate (2.64 g, 6.32 mmol) in THF (18 mL) and methanol (6 mL) at room temperature was added water (6 mL) followed by NaOH (1.27 g, 31.75 mmol) and the resulting mixture was stirred at room temperature for 3 days. The reaction mixture was quenched with an aqueous solution of 5% citric acid until pH=4 and the aqueous layer was extracted with ethyl acetate (3×50 mL). The combined organic layers were washed with brine (50 mL), dried over anhydrous sodium sulfate, filtered and concentrated under reduced pressure to afford 4-benzyloxy-6-(4-tert-butyl-2-methyl-phenyl)-2-methyl-pyridine-3-carboxylic acid (2.39 g, 93%) as a white solid. ESI-MS m/z calc. 389.19, found 390.2 (M+1)⁺. ¹H NMR (400 MHz, DMSO-d₆) δ 13.28 (br. s, 1H), 7.45-7.37 (m, 4H), 7.36-7.30 (m, 1H), 7.28 (s, 3H), 7.10 (s, 1H), 5.30 (s, 2H), 2.43 (s, 3H), 2.24 (s, 3H), 1.30 (s, 9H).

### Step 3: 4-benzyloxy-3-bromo-6-(4-tert-butyl-2-methyl-phenyl)-2-methyl-pyridine

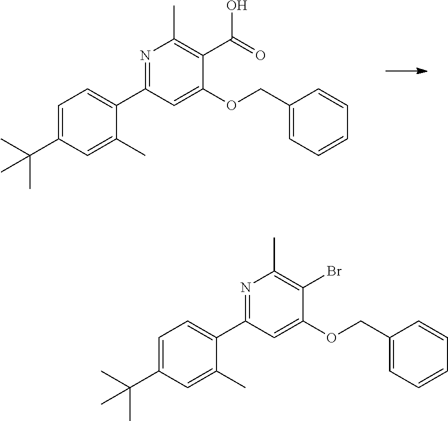 [SMILES: Cc1cc(C(C)(C)C)ccc1-c1cc(OCc2ccccc2)c(C(=O)O)c(C)n1 ; Cc1cc(C(C)(C)C)ccc1-c1cc(OCc2ccccc2)c(Br)c(C)n1]

A vial charged with 4-benzyloxy-6-(4-tert-butyl-2-methyl-phenyl)-2-methyl-pyridine-3-carboxylic acid (50 mg, 0.13 mmol), tripotassium phosphate (29 mg, 0.14 mmol) and tetrabutylammonium tribromide (98 mg, 0.20 mmol) was capped and purged with nitrogen. To the reaction vial was added acetonitrile (1 mL) via syringe and the reaction was stirred at 100° C. for 3 hours. The reaction mixture was diluted with MeOH (3 mL), filtered, and concentrated under reduced pressure to afford 4-benzyloxy-3-bromo-6-(4-tert-butyl-2-methyl-phenyl)-2-methyl-pyridine (80 mg, 100%) as an orange solid. ESI-MS m/z calc. 423.12, found 424.2 (M+1)⁺.

### Step 4: 3-[4-benzyloxy-6-(4-tert-butyl-2-methyl-phenyl)-2-methyl-3-pyridyl]pyridine-2-carbonitrile

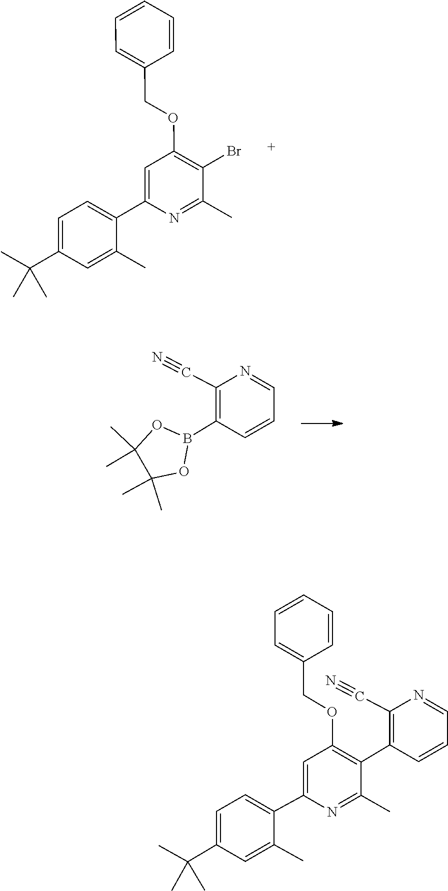 [SMILES: Cc1cc(C(C)(C)C)ccc1-c1cc(OCc2ccccc2)c(Br)c(C)n1 ; CC1(C)OB(c2cccnc2C#N)OC1(C)C ; Cc1cc(C(C)(C)C)ccc1-c1cc(OCc2ccccc2)c(-c2cccnc2C#N)c(C)n1]

In a vial, 4-benzyloxy-3-bromo-6-(4-tert-butyl-2-methyl-phenyl)-2-methyl-pyridine (20 mg, 0.047 mmol), 3-(4,4,5,5-tetramethyl-1,3,2-dioxaborolan-2-yl)pyridine-2-carbonitrile (21 mg, 0.09 mmol), SPhos Pd G3 (8 mg, 0.01 mmol), tripotassium phosphate (33 mg, 0.15 mmol), dioxane (600 μL), and water (60 L) were added. The resulting mixture was degassed under nitrogen and the vial was sealed and stirred at 80° C. for 16 h. It was cooled to room temperature, upon which a second portion of 3-(4,4,5,5-tetramethyl-1,3,2-dioxaborolan-2-yl)pyridine-2-carbonitrile (21 mg, 0.09 mmol) and SPhos Pd G3 (8 mg, 0.01 mmol) were added. This mixture was stirred at 80° C. for 3 days. It was then cooled to room temperature and diluted with EtOAc (2 mL). This mixture was washed with water (1 mL) and brine (1 mL), then dried over sodium sulfate, filtered, and evaporated in vacuo to give 3-[4-benzyloxy-6-(4-tert-butyl-2-methyl-phenyl)-2-methyl-3-pyridyl]pyridine-2-carbonitrile (3.2 mg, 15%). ESI-MS m/z calc. 447.23, found 448.5 (M+1)⁺.

### Step 5: 3-[6-(4-tert-butyl-2-methyl-phenyl)-2-methyl-4-oxo-1H-pyridin-3-yl]pyridine-2-carbonitrile (172)

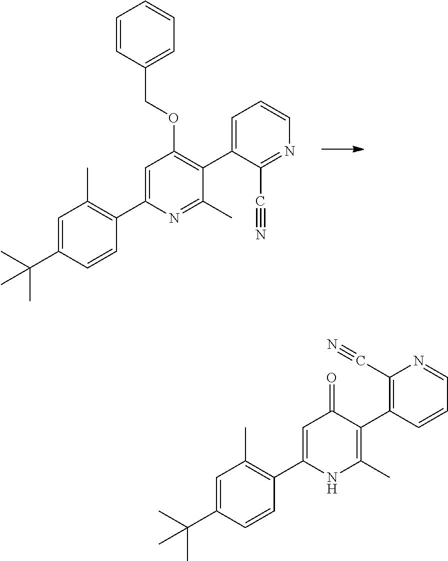 [SMILES: Cc1cc(C(C)(C)C)ccc1-c1cc(OCc2ccccc2)c(-c2cccnc2C#N)c(C)n1 ; Cc1cc(C(C)(C)C)ccc1-c1cc(=O)c(-c2cccnc2C#N)c(C)[nH]1]

3-[4-benzyloxy-6-(4-tert-butyl-2-methyl-phenyl)-2-methyl-3-pyridyl]pyridine-2-carbonitrile (14 mg, 0.03 mmol) and Pd on C, wet, Degussa (11 mg of 10% w/w, 0.01 mmol) was taken up in EtOH (1 mL). The reaction mixture was degassed under vacuum and a balloon filled with hydrogen was placed on the flask and the reaction mixture was stirred under an atmosphere of hydrogen for 15 minutes. The reaction mixture was filtered through a plug of celite and washed with methanol. The solvent was evaporated and the crude material was purified via silica gel column chromatography using 0 to 50 MeOH in DCM to obtain 3-[6-(4-tert-butyl-2-methyl-phenyl)-2-methyl-4-oxo-1H-pyridin-3-yl]pyridine-2-carbonitrile (172, 7 mg, 61%). ESI-MS m/z calc. 357.18, found 358.2 (M+1)⁺. ¹H NMR (400 MHz, DMSO-d₆) δ 11.64 (s, 1H), 8.71 (dd, J=4.7, 1.6 Hz, 1H), 7.95 (dd, J=8.0, 1.6 Hz, 1H), 7.78 (dd, J=8.0, 4.7 Hz, 1H), 7.41 (s, 1H), 7.38-7.31 (m, 2H), 6.12 (s, 1H), 2.33 (s, 3H), 2.11 (s, 3H), 1.32 (s, 9H).

The following compounds were synthesized using the route shown in Example 27 (Step 1 to Step 5), using the appropriate boronates with protecting groups if required. Suzuki coupling followed by deprotection provided the following compounds.

TABLE 6

| | | LC/MS | |
|---|---|---|---|
| | | (m/z calc.); | |
| Cmpd. | | Found | |
| No. | Compound Name | [M + H]⁺ | NMR (shifts in ppm) |
| 173 | 6-(4-tert-butyl-2-methyl- | 332.19 | ¹H NMR (400 MHz, DMSO-d₆) δ |
| | phenyl)-2-methyl-3-(3- | 333.3 | 13.73 (s, 1H), 8.73 (dd, J= 5.0, 1.6 |
| | pyridyl)-1H-pyridin-4-one | | Hz, 1H), 8.69 (d, J = 2.1 Hz, 1H), |
| | | | 8.04 (d, J = 8.0 Hz, 1H), 7.71 (dd, J = |
| | | | 7.9, 5.1 Hz, 1H), 7.49 (d, J = 1.9 Hz, |
| | | | 1H), 7.46 (dd, J = 8.0, 2.0 Hz, 1H), |
| | | | 7.41 (d, J = 8.0 Hz, 1H), 7.11 (s, 1H), |
| | | | 2.40 (s, 3H), 2.37 (s, 3H), 1.36 (s, 9H). |
| 174 | 6-(4-tert-butyl-2-methyl- | 350.24 | ¹H NMR (400 MHz, DMSO-d₆) δ |
| | phenyl)-2-methyl-3-(1- | 351.3 | 13.56 (s, 1H), 7.45 (s, 1H), 7.44- |
| | methyl-3,6-dihydro-2H- | | 7.39 (m, 1H), 7.35 (d, J = 8.0 Hz, |
| | pyridin-4-yl)-1H-pyridin-4- | | 1H), 6.98 (s, 1H), 5.76 (s, 1H), 4.05- |
| | one | | 3.84 (m, 2H), 3.82-3.68 (m, 2H), |
| | | | 3.56 (s, 3H), 2.88 (s, 3H), 2.82-2.68 |
| | | | (m, 2H), 2.31 (s, 3H), 1.34 (s, 9H). |
| 175 | 6-(4-tert-butyl-2-methyl- | 337.2 | ¹H NMR (400 MHz, DMSO-d₆) δ |
| | phenyl)-3-(3,6-dihydro-2H- | 338.5 | 14.03 (s, 1H), 7.47 (s, 1H), 7.43 (dd, |
| | pyran-4-yl)-2-methyl-1H- | | J = 8.1, 2.0 Hz, 1H), 7.38 (d, J = 8.0 |
| | pyridin-4-one | | Hz, 1H), 7.14 (s, 1H), 5.84 (s, 1H), |
| | | | 4.21 (d, J = 2.8 Hz, 2H), 3.84 (t, J = |
| | | | 5.4 Hz, 2H), 2.53 (s, 3H), 2.29 (s, |
| | | | 3H), 2.27-2.24 (m, 2H), 1.32 (s, 9H). |
| 176 | 6-(4-tert-butyl-5-chloro-2- | 366.15 | ¹H NMR (400 MHz, DMSO-d₆) δ |
| | methyl-phenyl)-2-methyl- | 367.2 | 11.57 (s, 1H), 8.60 (d, J = 5.0 Hz, |
| | 3-(4-pyridyl)-1H-pyridin-4- | | 2H), 7.45 (s, 1H), 7.42 (s, 1H), 7.32 |
| | one | | (d, J = 5.6 Hz, 2H), 6.20 (s, 1H), 2.32 |
| | | | (s, 3H), 2.16 (s, 3H), 1.48 (s, 9H). |
| 177 | 6-(4-tert-butyl-5-chloro-2- | 365.15 | ¹H NMR (400 MHz, DMSO-d₆) δ |
| | methyl-phenyl)-2-methyl- | 366.1 | 11.42 (s, 1H), 7.50-7.36 (m, 4H), 7.31 |
| | 3-phenyl-1H-pyridin-4-one | | (t, J = 7.3 Hz, 1H), 7.25 (s, 1H), 7.23 |
| | | | (s, 1H), 6.11 (s, 1H), 2.33 (s, 3H), |
| | | | 2.12 (s, 3H), 1.48 (s, 9H). [1] |
| 178 | 5-[6-(4-tert-butyl-5-chloro- | 413.15 | |
| | 2-methyl-phenyl)-2- | 414.4 | |
| | methyl-4-oxo-1H-pyridin- | | |
| | 3-yl]-2-methyl-pyrazole-3- | | |
| | carboxylic acid | | |
| 179 | 6-(4-tert-butyl-5-chloro-2- | 40514 | ¹H NMR (400 MHz, CD₃OD) δ 8.29 |
| | methyl-phenyl)-3-[1- | 406.3 | (s, 1H), 7.91 (s, 1H), 7.53 (t, J = 59.7 |
| | (difluoromethyl)pyrazol-4- | | Hz, 1H) 7.48 (s, 1H) and 7.41 (s, 1H), |
| | yl]-2-methyl-1H-pyridin-4- | | 6.49 (s, 1H), 2.46 (s, 3H), 2.32 (s, |
| | one | | 3H), 1.52 (s, 9H). |
| 180 | 6-(4-tert-butyl-5-chloro-2- | 399.17 | ¹H NMR (400 MHz, CD₃OD) δ 7.63 |
| | methyl-phenyl)-3-(3- | 400.4 | (s, 1H), 7.54 (s, 1H), 7.51 (s, 1H), |
| | methoxy-1-methyl-pyrazol- | | 7.02 (s, 1H), 3.93 (s, 3H), 3.84 (s, |
| | 4-yl)-2-methyl-1H-pyridin- | | 3H), 2.54 (s, 3H), 2.33 (s, 3H), 1.53 |
| | 4-one | | (s, 9H). |
| 181 | 6-(4-tert-butyl-5-chloro-2- | 406.16 | ¹H NMR (400 MHz, CD₃OD) δ 9.05 |
| | methyl-phenyl)-2-methyl- | 407.4 | (d, J = 1.4 Hz, 1H), 8.76 (dd, J = 4.8, |
| | 3-pyrazolo┌1,5-a┐pyrazin- | | 1.4 Hz, 1H), 8.31 (s, 1H), 8.02 (d, J = |
| | 3-yl-1H-pyridin-4-one | | 4.9 Hz, 1H), 7.58 (s, 2H), 7.18 (s, |
| | | | 1H), 2.63 (s, 3H), 2.41 (s, 3H), 1.54 |
| | | | (s, 9H). |
| 182 | 6-(4-tert-butyl-5-chloro-2- | 433.12 | ¹H NMR (400 MHz, CD₃OD) δ 8.39 |
| | methyl-phenyl)-2-methyl- | 434.3 | (s, 1H), 8.06 (s, 1H), 7.49 (s, 1H), |
| | 3-(1- | | 7.42 (s, 1H), 6.56 (s, 1H), 3.47 (s, |
| | methylsulfonylpyrazol-4- | | 3H), 2.48 (s, 3H), 2.33 (s, 3H), 1.52 |
| | yl)-1H-pyridin-4-one | | (s, 9H). |
| 183 | 6-(4-tert-butyl-5-chloro-2- | 395.18 | ¹H NMR (400 MHz, CD₃OD) δ 7.95 |
| | methyl-phenyl)-3-(1- | 396.5 | (s, 1H), 7.67 (s, 1H), 7.50 (s, 1H), |
| | cyclopropylpyrazol-4-yl)- | | 7.44 (s, 1H), 6.70 (s, 1H), 3.78-3.68 |
| | 2-methyl-1H-pyridin-4-one | | (m, 1H), 2.52 (s, 3H), 2.32 (s, 3H), |
| | | | 1.52 (s, 9H), 1.21-1.03 (m, 4H). |
| 184 | 6-(4-tert-butyl-5-chloro-2- | 404.12 | ¹H NMR (400 MHz, CD₃OD) δ 7.64 |
| | methyl-phenyl)-3-(5- | 404.4 | (s, 1H), 7.54 (s, 1H), 7.53 (s, 1H), |
| | chloro-1-methyl-pyrazol-4- | | 7.01 (s, 1H), 3.95 (s, 3H), 2.50 (s, |
| | yl)-2-methyl-1H-pyridin-4- | | 3H), 2.35 (s, 3H), 1.53 (s, 9H). |
| | one | | |
| 185 | 6-(4-tert-butyl-5-chloro-2- | 397.19 | ¹H NMR (400 MHz, CD₃OD) δ 8.01 |
| | methyl-phenyl)-3-(1- | 398.5 | (s, 1H), 7.76 (s, 1H), 7.55 (s, 1H), |
| | isopropylpyrazol-4-yl)-2- | | 7.52 (s, 1H), 7.05 (s, 1H), 4.63 (hept, |
| | methyl-1H-pyridin-4-one | | J = 6.6 Hz, 1H), 2.66 (s, 3H), 2.34 (s, |
| | | | 3H), 1.56 (d, J = 6.7 Hz, 6H), 1.53 (s, |
| | | | 9H). |
| 186 | 6-(4-tert-butyl-5-chloro-2- | 387.15 | ¹H NMR (400 MHz, CD₃OD) δ 7.57 |
| | methyl-phenyl)-3-(5- | 388.4 | (d, J = 3.1 Hz, 1H), 7.53 (s, 1H), 7.50 |
| | fluoro-1-methyl-pyrazol-4- | | (s, 1H), 6.91 (s, 1H), 3.83 (d, J = 1.2 |
| | yl)-2-methyl-1H-pyridin-4- | | Hz, 3H), 2.55 (s, 3H), 2.34 (s, 3H), |
| | one | | 1.52 (s, 9H). |
| 187 | 6-(4-tert-butyl-5-chloro-2- | 383.18 | ¹H NMR (400 MHz, CD₃OD) δ 8.01 |
| | methyl-phenyl)-3-(1- | 384.4 | (s, 1H), 7.76 (s, 1H), 7.55 (s, 1H), |
| | ethylpyrazol-4-yl)-2- | | 7.52 (s, 1H), 7.07 (s, 1H), 4.29 (q, J = |
| | methyl-1H-pyridin-4-one | | 7.3 Hz, 2H), 2.67 (s, 3H), 2.34 (s, |
| | | | 3H), 1.57-1.48 (m, 12H). |
| 188 | 5-[6-(4-tert-butyl-5-chloro- | 399.13 | ¹H NMR (400 MHz, CD₃OD) δ 7.80 |
| | 2-methyl-phenyl)-2- | 400.55 | (s, 1H), 7.47 (s, 1H), 7.40 (s, 1H), |
| | methyl-4-oxo-1H-pyridin- | | 6.38 (s, 1H), 2.61 (s, 3H), 2.32 (s, |
| | 3-yl]oxazole-2- | | 3H), 1.51 (s, 9H). |
| | carboxamide | | |
| 189 | 3-[1-(azetidin-3-yl)pyrazol- | 410.19 | ¹H NMR (400 MHz, CD₃OD) δ 8.10 |
| | 4-yl]-6-(4-tert-butyl-5- | 411.4 | (s, 1H), 7.98 (s, 1H), 7.55 (s, 1H), |
| | chloro-2-methyl-phenyl)-2- | | 7.53 (s, 1H), 7.13 (s, 1H), 5.56 (p, J = |
| | methyl-1H-pyridin-4-one | | 7.3 Hz, 1H), 4.68-4.55 (m, 4H), 2.69 |
| | | | (s, 3H), 2.34 (s, 3H), 1.53 (s, 9H). |
| 190 | 6-(4-tert-butyl-5-chloro-2- | 410.19 | ¹H NMR (400 MHz, CD₃OD) δ 7.77 |
| | methyl-phenyl)-2-methyl- | 411.4 | (s, 1H), 7.55 (s, 1H), 7.51 (s, 1H), |
| | 3-(4,5,6,7- | | 7.02 (s, 1H), 4.55 (t, J = 5.9 Hz, 2H), |
| | tetrahydropyrazolo[1,5- | | 4.47 (s, 2H), 3.89 (t, J = 5.9 Hz, 2H), |
| | a]pyrazin-3-yl)-1H-pyridin- | | 2.59 (s, 3H), 2.36 (s, 3H), 1.53 (s, |
| | 4-one | | 9H). |
| 191 | 6-(4-tert-butyl-5-chloro-2- | 423.13 | ¹H NMR (400 MHz, CD₃OD) δ 7.50 |
| | methyl-phenyl)-2-methyl- | 424.4 | (s, 1H), 7.43 (s, 1H), 6.79 (s, 1H), |
| | 3-[3-(trifluoromethyl)-1H- | | 6.57 (s, 1H), 2.48 (s, 3H), 2.33 (s, |
| | pyrazol-5-yl]-1H-pyridin- | | 3H), 1.52 (s, 9H). |
| | 4-one | | |
| 192 | 6-(5-chloro-2-methyl-4- | 423.13 | ¹H NMR (400 MHz, CD₃OD) δ 7.98 |
| | (1,1,1-trifluoro-2- | 424.4 | (s, 1H), 7.76 (s, 1H), 7.70 (s, 1H), |
| | methylpropan-2- | | 7.64 (s, 1H), 7.15 (s, 1H), 4.00 (s, |
| | yl)phenyl)-2-methyl-3-(1- | | 3H), 2.69 (s, 3H), 2.36 (s, 3H), 1.84 |
| | methyl-1H-pyrazol-4- | | (s, 6H). |
| | yl)pyridin-4(1H)-one | | |

### Example 28

### 6-(4-tert-butyl-5-chloro-2-methyl-phenyl)-2-methyl-3-pyrazin-2-yl-1H-pyridin-4-one (193)

### Step 1: 4-benzyloxy-6-chloro-3-iodo-2-methyl-pyridine

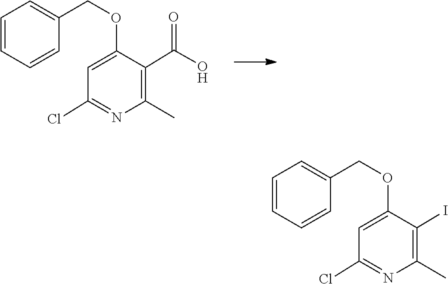 [SMILES: Cc1nc(Cl)cc(OCc2ccccc2)c1C(=O)O ; Cc1nc(Cl)cc(OCc2ccccc2)c1I]

A mixture of 4-benzyloxy-6-chloro-2-methyl-pyridine-3-carboxylic acid (25 mg, 0.09 mmol), iodine (91 mg, 0.36 mmol) and potassium phosphate (19 mg, 0.09 mmol) in anhydrous acetonitrile (0.6 mL) was heated at 100° C. for 2 days. After cooling to room temperature, the reaction mixture was diluted with a 10% aqueous solution of sodium thiosulfate and extracted with DCM (2×). The combined organics were washed with brine, dried over magnesium sulfate, filtered and concentrated under reduced pressure to give 4-benzyloxy-6-chloro-3-iodo-2-methyl-pyridine. ESI-MS m/z calc. 358.95, found 359.9 (M+1)⁺. ¹H-NMR (400 MHz, CDCl₃) δ 7.47-7.28 (m, 5H), 6.61 (s, 1H), 5.19 (s, 2H), 2.74 (s, 3H).

### Step 2: 2-(4-benzyloxy-6-chloro-2-methyl-3-pyridyl)pyrazine

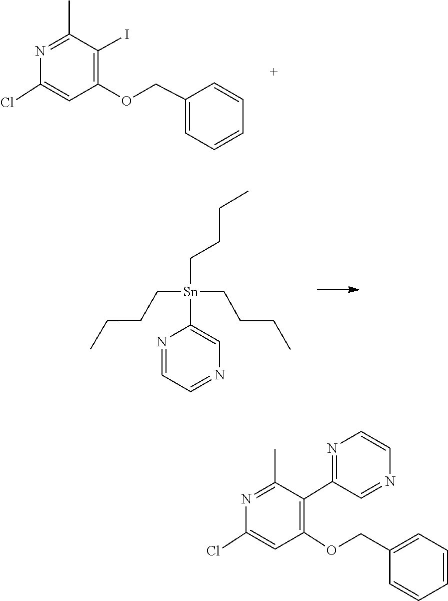 [SMILES: Cc1nc(Cl)cc(OCc2ccccc2)c1I ; CCC[CH2][Sn]([CH2]CCC)([CH2]CCC)[c]1cnccn1 ; Cc1nc(Cl)cc(OCc2ccccc2)c1-c1cnccn1]

A microwave vial charged with 4-benzyloxy-6-chloro-3-iodo-2-methyl-pyridine (50 mg, 0.14 mmol), tributyl(pyrazin-2-yl)stannane (66 mg. 0.18 mmol), Pd(PPh₃)₄(30 mg, 0.026 mmol) and CuI (15 mg, 0.08 mmol) and toluene (1.5 mL) was degassed for 30-60 seconds under a nitrogen atmosphere. The vial was sealed and stirred at 110° C. for 20 hours. The resulting mixture was diluted with ethyl acetate, washed with a saturated aqueous solution of ammonium chloride and then brine. The organic layer was dried over anhydrous magnesium sulfate, filtered, and concentrated under reduced pressure. Purification by silica gel column chromatography using 0 to 50% of EtOAc in hexanes gave 2-(4-benzyloxy-6-chloro-2-methyl-3-pyridyl)pyrazine (38 mg, 88%). ESI-MS m/z calc. 311.08, found 312.3 (M+1)⁺.

### Step 3: 2-[4-benzyloxy-6-(4-tert-butyl-5-chloro-2-methyl-phenyl)-2-methyl-3-pyridyl]pyrazine

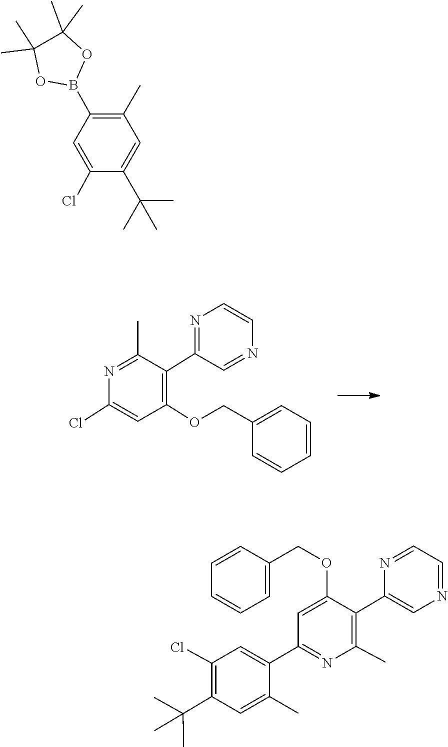 [SMILES: Cc1cc(C(C)(C)C)c(Cl)cc1B1OC(C)(C)C(C)(C)O1 ; Cc1nc(Cl)cc(OCc2ccccc2)c1-c1cnccn1 ; Cc1cc(C(C)(C)C)c(Cl)cc1-c1cc(OCc2ccccc2)c(-c2cnccn2)c(C)n1]

2-(4-benzyloxy-6-chloro-2-methyl-3-pyridyl)pyrazine (37 mg, 0.12 mmol), 2-(4-tert-butyl-5-chloro-2-methyl-phenyl)-4,4,5,5-tetramethyl-1,3,2-dioxaborolane (40 mg, 0.13 mmol), and tripotassium phosphate (350 μL of 1 M, 0.35 mmol) were combined in dioxane (1 mL) and purged with nitrogen for 1 minute. PdCl₂(dtbpf) (16 mg, 0.02 mmol) was added and the reaction was purged with nitrogen for an additional 1 minute, then sealed and stirred at room temperature for 30 minutes. The reaction was partitioned between ethyl acetate and water. The organics were separated, washed with brine, dried over magnesium sulfate, filtered and concentrated. The crude material was purified via silica gel column chromatography using 0 to 30% EtOAc in hexanes to give 2-[4-benzyloxy-6-(4-tert-butyl-5-chloro-2-methyl-phenyl)-2-methyl-3-pyridyl]pyrazine (27 mg, 50%). ESI-MS m/z calc. 457.19, found 458.5 (M+1)⁺.

### Step 4: 6-(4-tert-butyl-5-chloro-2-methyl-phenyl)-2-methyl-3-pyrazin-2-yl-1H-pyridin-4-one (193)

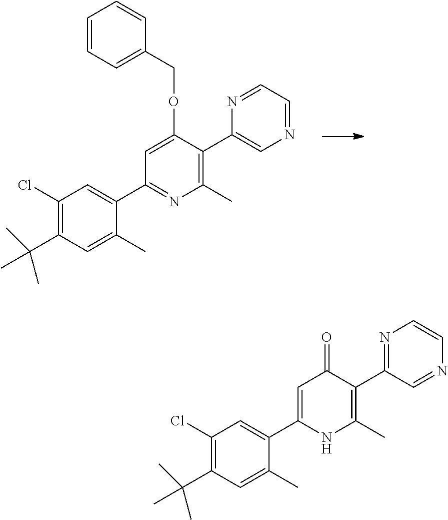 [SMILES: Cc1cc(C(C)(C)C)c(Cl)cc1-c1cc(OCc2ccccc2)c(-c2cnccn2)c(C)n1 ; Cc1cc(C(C)(C)C)c(Cl)cc1-c1cc(=O)c(-c2cnccn2)c(C)[nH]1]

To a solution of 2-[4-benzyloxy-6-(4-tert-butyl-5-chloro-2-methyl-phenyl)-2-methyl-3-pyridyl]pyrazine (37 mg, 0.08 mmol) in EtOH (1 mL) was added Pd/C, wet, Degussa (18 mg of 10% w/w, 0.017 mmol) and the reaction mixture was degassed under vacuum and backfilled with nitrogen. The reaction mixture was stirred under an atmosphere of hydrogen for 10 minutes using a balloon. The reaction mixture was filtered through a plug of celite and washed with methanol. The solvent was evaporated and the crude material was purified via silica gel column chromatography using 0 to 5% MeOH in DCM to obtain 6-(4-tert-butyl-5-chloro-2-methyl-phenyl)-2-methyl-3-pyrazin-2-yl-1H-pyridin-4-one (193, 29 mg, 94%). ESI-MS m/z calc. 367.14, found 368.1 (M+1)⁺. ¹H NMR (400 MHz, DMSO-d₆) δ 11.59 (s, 1H), 8.73 (d, J=1.5 Hz, 1H), 8.70 (d, J=2.4 Hz, 1H), 8.51 (d, J=2.6 Hz, 1H), 7.46 (s, 1H), 7.44 (s, 1H), 6.17 (s, 1H), 2.32 (s, 3H), 2.21 (s, 3H), 1.48 (s, 9H).

The following compounds were synthesized using the route analogous to that shown in Example 28 (Step 2 to Step 4), using the appropriate stannanes. Stillie coupling followed by Suzuki coupling and deprotection provided the following compounds.

TABLE 7

| | | LC/MS | |
|---|---|---|---|
| | | (m/z calc.); | |
| Cmpd. | | Found | |
| No. | Compound Name | [M + H]⁺ | NMR (shifts in ppm) |
| 194 | 6-(4-tert-butyl-5-chloro-2- | 396.16 | ¹H NMR (400 MHz, CD₃OD) δ 7.76 |
| | methyl-phenyl)-3-(6- | 397.4 | (dd, J = 8.4, 7.3 Hz, 1H), 7.49 (s, |
| | methoxy-2-pyridyl)-2- | | 1H), 7.42 (s, 1H), 7.08 (dd, J = 7.3, |
| | methyl-1H-pyridin-4-one | | 0.8 Hz, 1H), 6.79 (dd, J = 8.4, 0.8 Hz, |
| | | | 1H), 6.49 (s, 1H), 3.92 (s, 3H), 2.35 |
| | | | (s, 3H), 2.30 (s, 3H), 1.52 (s, 9H) |
| 195 | 6-(4-tert-butyl-5-chloro-2- | 382.14 | ¹H NMR (400 MHz, CD₃OD) δ 7.85 |
| | methyl-phenyl)-2-methyl- | 383.3 | (dd, J = 9.2, 6.9 Hz, 1H), 7.57 (s, |
| | 3-(6-oxo-1H-pyridin-2-yl)- | | 1H), 7.54 (s, 1H), 7.15 (s, 1H), 6.78 |
| | 1H-pyridin-4-one | | (dd, J = 9.2, 1.0 Hz, 1H), 6.69 (dd, |
| | | | J = 6.9, 1.0 Hz, 1H), 2.58 (s, 3H), 2.36 |
| | | | (s, 3H), 1.53 (s, 9H) |
| 196 | 6-(4-tert-butyl-5-chloro-2- | 397.16 | ¹H NMR (400 MHz, CD₃OD) δ 8.34- |
| | methyl-phenyl)-3-(3- | 398.5 | 8.27 (m, 2H), 7.53 (s, 1H), 7.52 (s, |
| | methoxypyrazin-2-yl)-2- | | 1H), 6.81 (s, 1H), 3.99 (s, 3H), 2.36 |
| | methyl-1H-pyridin-4-one | | (s, 3H), 2.31 (s, 3H), 1.53 (s, 9H). |
| 197 | 6-(4-tert-butyl-5-chloro-2- | 383.18 | ¹H NMR (400 MHz, CD₃OD) δ 7.80 |
| | methyl-phenyl)-3-(1,2- | 384.52 | (s, 1H), 7.57 (s, 1H), 7.56 (s, 1H), |
| | dimethylimidazol-4-yl)-2- | | 7.19 (s, 1H), 3.91 (s, 3H), 2.73 (s, |
| | methyl-1H-pyridin-4-one | | 3H), 2.71 (s, 3H), 2.36 (s, 3H), 1.53 |
| | | | (s, 9H). |
| 198 | 6-(4-tert-butyl-5-chloro-2- | 369.16 | ¹H NMR (400 MHz, CD₃OD) δ 7.85 |
| | methyl-phenyl)-2-methyl- | 370.5 | (d, J = 2.4 Hz, 1H), 7.58-7.53 (m, |
| | 3-(1-methylpyrazol-3-yl)- | | 2H), 7.23 (s, 1H), 6.72 (d, J = 2.4 Hz, |
| | 1H-pyridin-4-one | | 1H), 4.04 (s, 3H), 2.76 (s, 3H), 2.35 |
| | | | (s, 3H), 1.53 (s, 9H). |
| 199 | 6-(4-tert-butyl-5-chloro-2- | 370.16 | ¹H NMR (400 MHz, CD₃OD) δ 8.02 |
| | methyl-phenyl)-2-methyl- | 371.4 | (s, 1H), 7.51 (s, 1H), 7.47 (s, 1H), |
| | 3-(2-methyltriazol-4-yl)- | | 6.75 (s, 1H), 4.26 (s, 3H), 2.61 (s, |
| | 1H-pyridin-4-one | | 3H), 2.33 (s, 3H), 1.52 (s, 9H). |
| 200 | 6-(4-tert-butyl-5-chloro-2- | 371.15 | ¹H NMR (400 MHz, CD₃OD) δ 7.51 |
| | methyl-phenyl)-2-methyl- | 372.5 | (s, 1H), 7.48 (s, 1H), 6.65 (s, 1H), |
| | 3-(2-methyltetrazol-5-yl)- | | 4.47 (s, 3H), 2.40 (s, 3H), 2.35 (s, |
| | 1H-pyridin-4-one | | 3H), 1.52 (s, 9H) |
| 201 | 6-(4-tert-butyl-5-chloro-2- | 399.17 | ¹H NMR (400 MHz, CD₃OD) δ 7.95 |
| | methyl-phenyl)-3-[1- | 400.66 | (d, J = 2.1 Hz, 1H), 7.82 (d, J = 2.1 |
| | (methoxymethyl)imidazol- | | Hz, 1H), 7.51 (s, 1H), 7.47 (s, 1H), |
| | 2-yl]-2-methyl-1H-pyridin- | | 6.53 (s, 1H), 5.44 (s, 2H), 3.40 (s, |
| | 4-one | | 3H), 2.35 (s, 3H), 2.33 (s, 3H), 1.52 |
| | | | (s, 9H). |
| 202 | 6-(4-tert-butyl-5-chloro-2- | 357.14 | ¹H NMR (400 MHz, CD₃OD) δ 7.49 |
| | methyl-phenyl)-2-methyl- | 358.4 | (s, 1H), 7.44 (s, 1H), 6.49 (s, 1H), |
| | 3-(2H-tetrazol-5-yl)-1H- | | 2.79 (s, 3H), 2.33 (s, 3H), 1.52 (s, |
| | pyridin-4-one | | 9H). |
| 203 | 6-(4-(tert-butyl)-5-chloro- | 369.16 | ¹H NMR (400 MHz, CD₃OD) δ 9.13 |
| | 2-methylphenyl)-2-methyl- | 370.4 | (d, J = 2.2 Hz, 1H), 7.74 (d, J = 1.5 |
| | 3-(1-methyl-1H-imidazol- | | Hz, 1H), 7.55 (s, 1H), 7.51 (s, 1H), |
| | 5-yl)pyridin-4(1H)-one | | 6.86 (s, 1H), 3.80 (s, 3H), 2.48 (s, |
| | | | 3H), 2.37 (s, 3H), 1.53 (s, 9H). |

### Example 29

### 6-(4-tert-butyl-2-methyl-phenyl)-2-methyl-3-(1-methyl-4-piperidyl)-1H-pyridin-4-one (204)

### Step 1: 6-(4-tert-butyl-2-methyl-phenyl)-2-methyl-3-(1-methyl-4-piperidyl)-1H-pyridin-4-one (204)

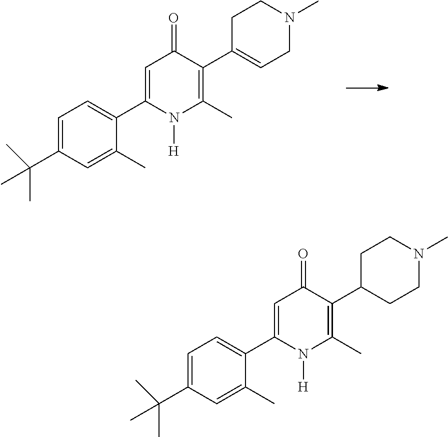

To a stirring solution of 6-(4-tert-butyl-2-methyl-phenyl)-2-methyl-3-(1-methyl-3,6-dihydro-2H-pyridin-4-yl)-1H-pyridin-4-one (15 mg, 0.04 mmol) in Methanol (5 mL) Pd/C (5 mg of 10% w/v) was added. The reaction mixture was stirred under an atmosphere of hydrogen for 3 days at room temperature using a balloon. The reaction mixture was filtered and purified by reverse phase preparative chromatography (C₁₈) using 5 to 50% acetonitrile in water containing 5 mM hydrochloric acid to give 6-(4-tert-butyl-2-methyl-phenyl)-2-methyl-3-(1-methyl-4-piperidyl)-1H-pyridin-4-one (204, approximately 11.3 mg, 75%). ESI-MS m/z calc. 352.25, found 353.4 (M+1)⁺. ¹H NMR (400 MHz, DMSO-d₆) δ 10.03 (s, 1H), 7.45 (s, 1H), 7.42 (dd, J=8.0, 2.0 Hz, 1H), 7.34 (d, J=8.0 Hz, 1H), 7.08 (s, 1H), 3.50 (d, J=12.0 Hz, 2H), 3.12-3.02 (m, 3H), 2.77 (d, J=4.3 Hz, 3H), 2.70 (d, J=3.6 Hz, 2H), 2.65 (s, 3H), 2.28 (s, 3H), 1.78 (d, J=13.9 Hz, 2H), 1.34 (s, 9H).

### Example 30

### 6-(4-tert-butyl-2-methyl-phenyl)-2-methyl-3-(1,2,3,6-tetrahydropyridin-4-yl)-1H-pyridin-4-one (205)

### Step 1: tert-butyl 4-[6-(4-tert-butyl-2-methyl-phenyl)-2-methyl-4-oxo-1H-pyridin-3-yl]-3,6-dihydro-2H-pyridine-1-carboxylate

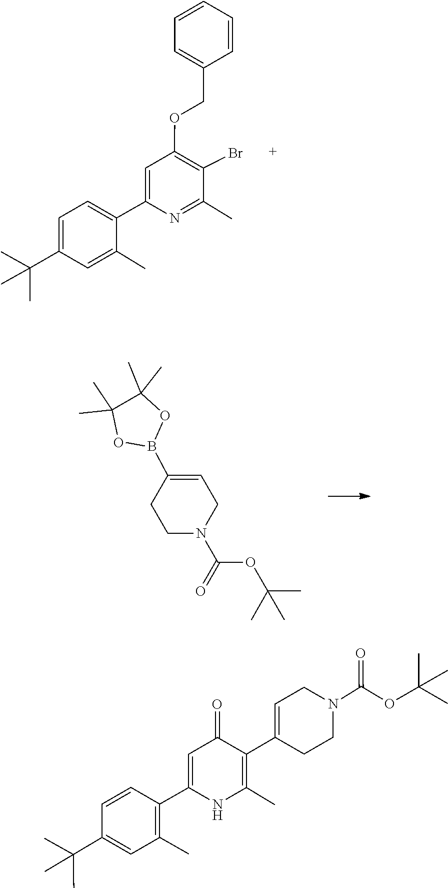 [SMILES: Cc1cc(C(C)(C)C)ccc1-c1cc(OCc2ccccc2)c(Br)c(C)n1 ; CC(C)(C)OC(=O)N1CC=C(B2OC(C)(C)C(C)(C)O2)CC1 ; Cc1cc(C(C)(C)C)ccc1-c1cc(=O)c(C2=CCN(C(=O)OC(C)(C)C)CC2)c(C)[nH]1]

tert-butyl 4-[4-benzyloxy-6-(4-tert-butyl-2-methyl-phenyl)-2-methyl-3-pyridyl]-3,6-dihydro-2H-pyridine-1-carboxylate was prepared from tert-butyl 4-(4,4,5,5-tetramethyl-1,3,2-dioxaborolan-2-yl)-3,6-dihydro-2H-pyridine-1-carboxylate, using procedure analogous to that found in Example 27 (Step 4 and Step 5). ESI-MS m/z calc. 526.32, found 527.0 (M+1)⁺.

### Step 2: 6-(4-tert-butyl-2-methyl-phenyl)-2-methyl-3-(1,2,3,6-tetrahydropyridin-4-yl)-1H-pyridin-4-one (205)

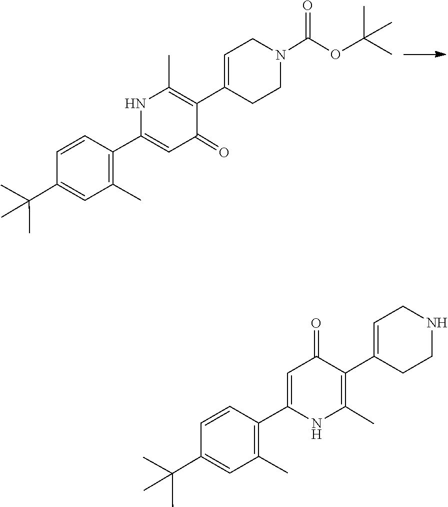

tert-butyl 4-[6-(4-tert-butyl-2-methyl-phenyl)-2-methyl-4-oxo-1H-pyridin-3-yl]-3,6-dihydro-2H-pyridine-1-carboxylate (20 mg, 0.045 mmol) was added TFA in DCM (1:1) (5 mL). The mixture was stirred at room temperature for 30 min. The reaction was concentrated and purified by reverse phase preparative chromatography (C₁₈) using 5 to 40% acetonitrile in water containing 5 mM hydrochloric acid to give 6-(4-tert-butyl-2-methyl-phenyl)-2-methyl-3-(1,2,3,6-tetrahydropyridin-4-yl)-1H-pyridin-4-one (205, 2.3 mg, 7%). ¹H NMR (400 MHz, Methanol-d₄) δ 7.52-7.41 (m, 2H), 7.40-7.31 (m, 1H), 6.94 (s, 1H), 5.90 (s, 1H), 3.90 (s, 2H), 3.52 (t, J=6.2 Hz, 3H), 2.66 (s, 2H), 2.61 (s, 3H), 2.34 (s, 3H), 1.36 (d, J=1.8 Hz, 9H). ESI-MS m/z calc. 336.22, found 337.3 (M+1)⁺.

### Example 31

### 5-[6-(4-tert-butyl-5-chloro-2-methyl-phenyl)-2-methyl-4-oxo-1H-pyridin-3-yl]-1H-pyrimidine-2,4-dione (206)

### Step 1: 2,4-dibenzyloxy-5-(4-benzyloxy-6-chloro-2-methyl-3-pyridyl)pyrimidine

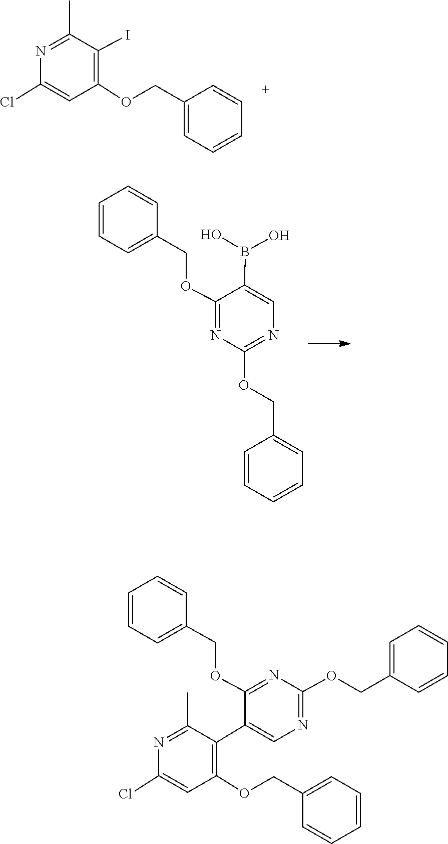 [SMILES: Cc1nc(Cl)cc(OCc2ccccc2)c1I ; OB(O)c1cnc(OCc2ccccc2)nc1OCc1ccccc1 ; Cc1nc(Cl)cc(OCc2ccccc2)c1-c1cnc(OCc2ccccc2)nc1OCc1ccccc1]

A microwave vial was charged with 4-benzyloxy-6-chloro-3-iodo-2-methyl-pyridine (115 mg, 0.32 mmol), (2,4-dibenzyloxypyrimidin-5-yl)boronic acid (125 mg, 0.35 mmol), PdCl₂(dtbpf) (38 mg, 0.06 mmol), and potassium phosphate (325 mg, 1.53 mmol). Dioxane (3 mL) and water (1 mL) were added and the reaction mixture was degassed under nitrogen. The tube was sealed and stirred at 45° C. for 7 hours. The resulting mixture was diluted with ethyl acetate, washed with a saturated aqueous solution of ammonium chloride and then brine. The organic layer was dried over anhydrous magnesium sulfate, filtered, and concentrated under reduced pressure. Purification by silica gel chromatography using 0 to 50% of EtOAc in hexanes gave 2,4-dibenzyloxy-5-(4-benzyloxy-6-chloro-2-methyl-3-pyridyl)pyrimidine (120 mg, 65%). ¹H NMR (400 MHz, CD₃OD) δ 8.15 (s, 1H), 7.48-7.43 (m, 2H), 7.39-7.32 (m, 3H), 7.29-7.20 (m, 8H), 7.20-7.13 (m, 2H), 7.08 (s, 1H), 5.48-5.38 (m, 4H), 5.15 (d, J=12.2 Hz, 1H), 5.06 (d, J=12.2 Hz, 1H), 2.20 (s, 3H). ESI-MS m/z calc. 523.16, found 524.6 (M+1)⁺.

### Step 2: 2,4-dibenzyloxy-5-[4-benzyloxy-6-(4-tert-butyl-5-chloro-2-methyl-phenyl)-2-methyl-3-pyridyl]pyrimidine

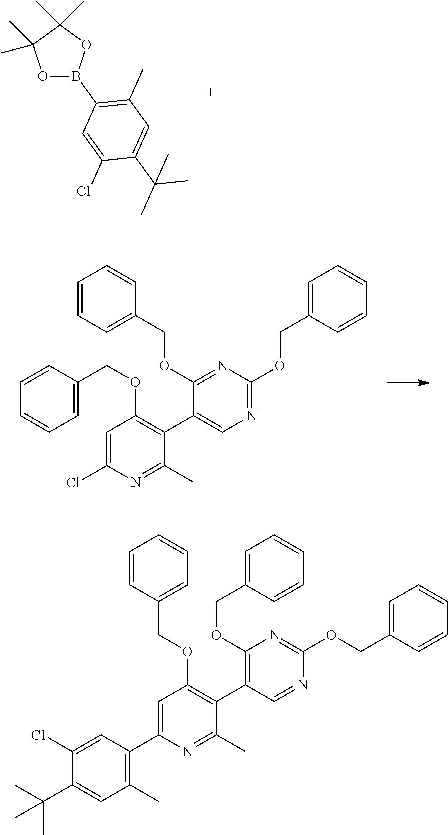 [SMILES: Cc1cc(C(C)(C)C)c(Cl)cc1B1OC(C)(C)C(C)(C)O1 ; Cc1nc(Cl)cc(OCc2ccccc2)c1-c1cnc(OCc2ccccc2)nc1OCc1ccccc1 ; Cc1cc(C(C)(C)C)c(Cl)cc1-c1cc(OCc2ccccc2)c(-c2cnc(OCc3ccccc3)nc2OCc2ccccc2)c(C)n1]

A microwave vial was charged with 2-(4-tert-butyl-5-chloro-2-methyl-phenyl)-4,4,5,5-tetramethyl-1,3,2-dioxaborolane (20 mg, 0.06 mmol), 2,4-dibenzyloxy-5-(4-benzyloxy-6-chloro-2-methyl-3-pyridyl)pyrimidine (30 mg, 0.05 mmol), PdCl₂(dtbpf) (7 mg, 0.01 mmol), and potassium phosphate (55 mg, 0.26 mmol). Dioxane (1 μL) and water (300 μL) were then added and the reaction mixture was degassed under nitrogen. The tube was sealed and stirred at 45° C. for 2 hours. The reaction mixture was diluted with ethyl acetate, washed with a saturated aqueous solution of ammonium chloride and then brine. The organic layer was dried over anhydrous magnesium sulfate, filtered, and concentrated under reduced pressure. Purification by silica gel chromatography using 0 to 70% of EtOAc in hexanes gave 2,4-dibenzyloxy-5-[4-benzyloxy-6-(4-tert-butyl-5-chloro-2-methyl-phenyl)-2-methyl-3-pyridyl]pyrimidine. ESI-MS m/z calc. 669.27, found 670.8 (M+1)⁺.

### Step 3: 5-[6-(4-tert-butyl-5-chloro-2-methyl-phenyl)-2-methyl-4-oxo-1H-pyridin-3-yl]-1H-pyrimidine-2,4-dione (206)

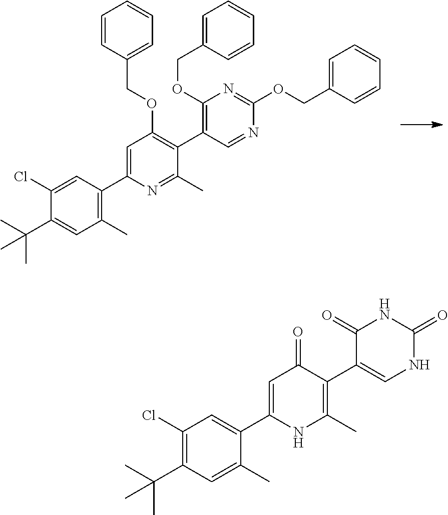 [SMILES: Cc1cc(C(C)(C)C)c(Cl)cc1-c1cc(OCc2ccccc2)c(-c2cnc(OCc3ccccc3)nc2OCc2ccccc2)c(C)n1 ; Cc1cc(C(C)(C)C)c(Cl)cc1-c1cc(=O)c(-c2c[nH]c(=O)[nH]c2=O)c(C)[nH]1]

2,4-Dibenzyloxy-5-[4-benzyloxy-6-(4-tert-butyl-5-chloro-2-methyl-phenyl)-2-methyl-3-pyridyl]pyrimidine was dissolved in MeOH (4 mL). Then Palladium 10% on C (wet) (8 mg, 0.004 mmol) was added and the resulting mixture was placed under hydrogen atmosphere and stirred at 25° C. for 15 minutes. The solvent was removed and the reaction mixture was purified by reverse phase HPLC (C₁₈) using 10-99% acetonitrile in water containing 5 mM HCl to yield 5-[6-(4-tert-butyl-5-chloro-2-methyl-phenyl)-2-methyl-4-oxo-1H-pyridin-3-yl]-1H-pyrimidine-2,4-dione (206, 12 mg, 58%). EST-MS m/z calc. 399.13, found 400.4 (M+1)⁺. ¹H NMR (400 MHz, CD₃OD) δ 7.53 (s, 1H), 7.51 (s, 1H), 7.46 (s, 1H), 6.78 (s, 1H), 2.43 (s, 3H), 2.33 (s, 3H), 1.52 (s, 9H).

The following compounds were synthesized using the route analogous to that shown in Example 31 (Step 1 to Step 3), using the appropriate boronates with protecting groups if required. Suzuki coupling followed by a second Suzuki coupling and deprotection provided the following compounds.

TABLE 8

| | | LC/MS | |
|---|---|---|---|
| | | (m/z calc.); | |
| Cmpd. | | Found | |
| No. | Compound Name | [M + H]⁺ | NMR (shifts in ppm) |
| 207 | 6-(4-tert-butyl-5-chloro-2- | 370.14 | ¹H NMR (400 MHz, CD₃OD) δ 7.47 |
| | methyl-phenyl)-2-methyl- | 371.5 | (s, 1H), 7.40 (s, 1H), 6.75 (s, 1H), |
| | 3-(3-methylisoxazol-5-yl)- | | 6.37 (s, 1H), 2.50 (s, 3H), 2.36 (s, |
| | 1H-pyridin-4-one | | 3H), 2.32 (s, 3H), 1.51 (s, 9H) |
| 208 | 4-[6-(4-tert-butyl-5-chloro- | 394.16 | ¹H NMR (400 MHz, CD₃OD) δ 7.64 |
| | 2-methyl-phenyl)-2- | 395.4 | (s, 1H), 7.47 (s, 1H), 7.42 (s, 1H), |
| | methyl-4-oxo-1H-pyridin- | | 6.37 (s, 1H), 4.10 (s, 3H), 2.35 (s, |
| | 3-yl]-2-methyl-pyrazole-3- | | 3H), 2.33 (s, 3H), 1.51 (s, 9H) |
| | carbonitrile | | |
| 209 | 6-(6-tert-butyl-5-chloro-2- | 370.16 | ¹H NMR (400 MHz, CD₃OD) δ 7.99 |
| | methyl-3-pyridyl)-2- | 371.4 | (s, 1H), 7.92 (s, 1H), 7.77 (s, 1H), |
| | methyl-3-(1- | | 7.19 (s, 1H), 4.00 (s, 3H), 2.70 (s, |
| | methylpyrazol-4-yl)-1H- | | 3H), 2.52 (s, 3H), 1.54 (s, 9H). |
| | pyridin-4-one | | |
| 210 | 6-(4-tert-butyl-5-chloro-2- | 355.15 | ¹H NMR (400 MHz, CD₃OD) δ 7.88 |
| | methyl-phenyl)-2-methyl- | 356.4 | (s, 2H), 7.52 (s, 1H), 7.47 (s, 1H), |
| | 3-(1H-pyrazol-4-yl)-1H- | | 6.80 (s, 1H), 2.57 (s, 3H), 2.33 (s, |
| | pyridin-4-one | | 3H), 1.52 (s, 9H) |
| 211 | 4-[6-(4-tert-butyl-5-chloro- | 394.16 | ¹H NMR (400 MHz, CD₃OD) δ 7.81 |
| | 2-methyl-phenyl)-2- | 395.3 | (s, 1H), 7.47 (s, 1H), 7.41 (s, 1H), |
| | methyl-4-oxo-1H-pyridin- | | 6.37 (s, 1H), 4.02 (s, 3H), 2.35-2.30 |
| | 3-yl]-1-methyl-pyrazole-3- | | (m, 6H), 1.51 (s, 9H) |
| | carbonitrile | | |
| 212 | 6-(4-tert-butyl-5-chloro-2- | 355.15 | ¹H NMR (400 MHz, CD₃OD) δ 7.78 |
| | methyl-phenyl)-3-(1H- | 356.52 | (s, 2H), 7.54 (s, 1H), 7.52 (s, 1H), |
| | imidazol-2-yl)-2-methyl- | | 6.87 (s, 1H), 2.57 (s, 3H), 2.36 (s, |
| | 1H-pyridin-4-one | | 3H), 1.53 (s, 9H). |

### Example 32

### 6-[6-(4-tert-butyl-5-chloro-2-methyl-phenyl)-2-methyl-4-oxo-1H-pyridin-3-yl]pyridine-2-carboxamide (213)

### Step 1: 6-[6-(4-tert-butyl-5-chloro-2-methyl-phenyl)-2-methyl-4-oxo-1H-pyridin-3-yl]pyridine-2-carbonitrile

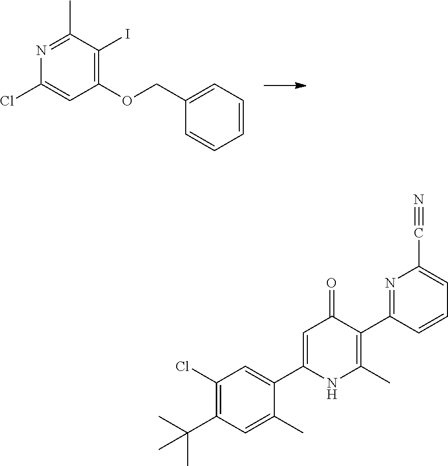 [SMILES: Cc1nc(Cl)cc(OCc2ccccc2)c1I ; Cc1cc(C(C)(C)C)c(Cl)cc1-c1cc(=O)c(-c2cccc(C#N)n2)c(C)[nH]1]

6-[6-(4-tert-butyl-5-chloro-2-methyl-phenyl)-2-methyl-4-oxo-1H-pyridin-3-yl]pyridine-2-carbonitrile was prepared from 6-(4,4,5,5-tetramethyl-1,3,2-dioxaborolan-2-yl)pyridine-2-carbonitrile using a procedure analogous to that found in Example 31 (Step 1 to Step 3).

### Step 2: 6-[6-(4-tert-butyl-5-chloro-2-methyl-phenyl)-2-methyl-4-oxo-1H-pyridin-3-yl]pyridine-2-carboxamide (213)

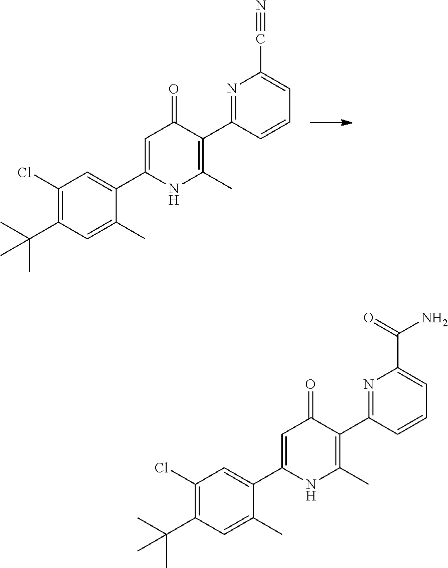 [SMILES: Cc1cc(C(C)(C)C)c(Cl)cc1-c1cc(=O)c(-c2cccc(C#N)n2)c(C)[nH]1 ; Cc1cc(C(C)(C)C)c(Cl)cc1-c1cc(=O)c(-c2cccc(C(N)=O)n2)c(C)[nH]1]

6-[6-(4-tert-butyl-5-chloro-2-methyl-phenyl)-2-methyl-4-oxo-1H-pyridin-3-yl]pyridine-2-carbonitrile was dissolved in DMSO (875 L) and potassium carbonate (43 mg, 0.31 mmol) and hydrogen peroxide (150 μL of 30% w/v, 1.32 mmol) were added. The reaction mixture was stirred at room temperature for 4 hours. Purification by high pressure reverse phase chromatography (C₁₈) using 1 to 100% ACN in water containing 5 mM hydrochloric acid gave 6-[6-(4-tert-butyl-5-chloro-2-methyl-phenyl)-2-methyl-4-oxo-1H-pyridin-3-yl]pyridine-2-carboxamide (213, 6 mg, 19%) as a white solid. ESI-MS m/z calc. 409.15, found 410.7 (M+1)⁺. ¹H NMR (400 MHz, MeOD) δ 8.23 (dd, J=7.9, 1.2 Hz, 1H), 8.16 (t, J=7.7 Hz, 1H), 7.82 (dd, J=7.6, 1.2 Hz, 1H), 7.58 (s, 1H), 7.57 (s, 1H), 7.19 (s, 1H), 2.55 (s, 3H), 2.38 (s, 3H), 1.54 (s, 9H).

### Example 33

### 6-(4-tert-butyl-5-chloro-2-methyl-phenyl)-2-methyl-3-tetrahydropyran-4-yl-1H-pyridin-4-one (214)

### Step 1: 6-(4-tert-butyl-5-chloro-2-methyl-phenyl)-3-(3,6-dihydro-2H-pyran-4-yl)-2-methyl-1H-pyridin-4-one

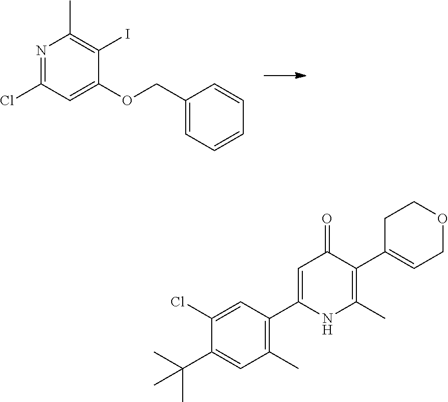 [SMILES: Cc1nc(Cl)cc(OCc2ccccc2)c1I ; Cc1cc(C(C)(C)C)c(Cl)cc1-c1cc(=O)c(C2=CCOCC2)c(C)[nH]1]

6-(4-tert-butyl-5-chloro-2-methyl-phenyl)-3-(3,6-dihydro-2H-pyran-4-yl)-2-methyl-1H-pyridin-4-one was prepared from 2-(3,6-dihydro-2H-pyran-4-yl)-4,4,5,5-tetramethyl-1,3,2-dioxaborolane using procedure analogous to that found in Example 31 (Step 1 to Step 3). ESI-MS m/z calc. 371.16, found 372.6 (M+1)⁺.

### Step 2: 6-(4-tert-butyl-5-chloro-2-methyl-phenyl)-2-methyl-3-tetrahydropyran-4-yl-1H-pyridin-4-one (214)

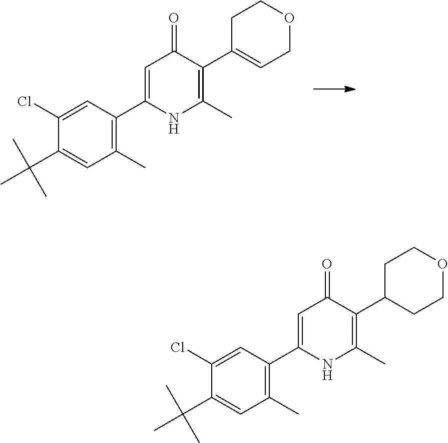 [SMILES: Cc1cc(C(C)(C)C)c(Cl)cc1-c1cc(=O)c(C2=CCOCC2)c(C)[nH]1 ; Cc1cc(C(C)(C)C)c(Cl)cc1-c1cc(=O)c(C2CCOCC2)c(C)[nH]1]

6-(4-tert-butyl-5-chloro-2-methyl-phenyl)-2-methyl-3-tetrahydropyran-4-yl-1H-pyridin-4-one (214) was prepared from 6-(4-tert-butyl-5-chloro-2-methyl-phenyl)-3-(3,6-dihydro-2H-pyran-4-yl)-2-methyl-1H-pyridin-4-one using procedure analogous to that found in Example 29. ESI-MS m/z calc. 373.18, found 374.4 (M+1)⁺. ¹H NMR (400 MHz, MeOD) δ 7.52 (s, 1H), 7.46 (s, 1H), 6.94 (s, 1H), 4.06 (dd, J=11.4, 4.4 Hz, 2H), 3.65-3.50 (m, 2H), 3.28-3.17 (m, 1H), 2.71 (s, 3H), 2.64-2.49 (m, 2H), 2.29 (s, 3H), 1.59-1.53 (m, 2H), 1.52 (s, 9H).

### Example 34

### 4-[6-(4-tert-butyl-5-chloro-2-methyl-phenyl)-2-methyl-4-oxo-1H-pyridin-3-yl]pyrimidine-2-carboxamide (215)

### Step 1: 4-(4-benzyloxy-6-chloro-2-methyl-3-pyridyl)-2-chloro-pyrimidine

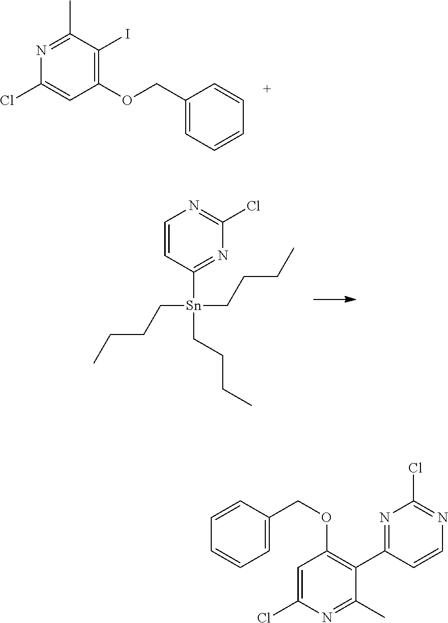 [SMILES: Cc1nc(Cl)cc(OCc2ccccc2)c1I ; CCC[CH2][Sn]([CH2]CCC)([CH2]CCC)[c]1ccnc(Cl)n1 ; Cc1nc(Cl)cc(OCc2ccccc2)c1-c1ccnc(Cl)n1]

A microwave vial was charged with 4-benzyloxy-6-chloro-3-iodo-2-methyl-pyridine (200 mg, 0.55 mmol), tributyl-(2-chloropyrimidin-4-yl)stannane (293 mg, 0.72 mmol), Pd(PPh₃)₄(115 mg, 0.1 mmol) CuI (22 mg, 0.11 mmol) and toluene (6 mL). The reaction mixture was degassed for 30-60 seconds under an atmosphere of nitrogen. The vial was sealed and stirred at 110° C. for 20 hours. The resulting mixture was diluted with ethyl acetate, washed with a saturated aqueous solution of ammonium chloride and brine. The organic layer was dried over anhydrous magnesium sulfate, filtered, and concentrated under reduced pressure. Purification by silica gel chromatography using 0 to 50% of EtOAc in hexanes afforded the desired product 4-(4-benzyloxy-6-chloro-2-methyl-3-pyridyl)-2-chloro-pyrimidine (126 mg, 65%). ESI-MS m/z calc. 345.04, found 348.2 (M+3)⁺. ¹H NMR (400 MHz, DMSO-d₆) δ 8.88 (d, J=5.0 Hz, 1H), 7.75 (d, J=5.0 Hz, 1H), 7.39-7.27 (m, 6H), 5.26 (s, 2H), 2.26 (s, 3H).

### Step 2: 4-(4-benzyloxy-6-chloro-2-methyl-3-pyridyl)pyrimidine-2-carbonitrile

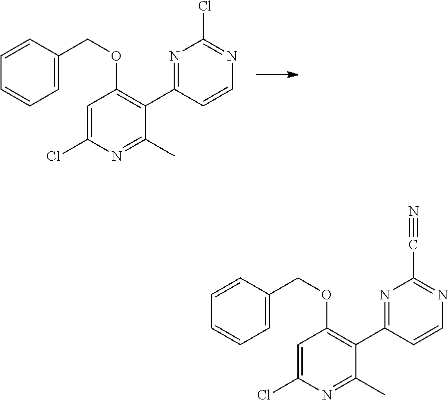 [SMILES: Cc1nc(Cl)cc(OCc2ccccc2)c1-c1ccnc(Cl)n1 ; Cc1nc(Cl)cc(OCc2ccccc2)c1-c1ccnc(C#N)n1]

A vial charged with 4-(4-benzyloxy-6-chloro-2-methyl-3-pyridyl)-2-chloro-pyrimidine (110 mg, 0.32 mmol), sodium cyanide (25 mg, 0.51 mmol), DABCO (5 mg, 0.04 mmol), DMSO (500 L) and water (250 mL) was stirred at room temperature for 16 hours. The reaction mixture was quenched with water and the aqueous layer was extracted with ethyl acetate (2×). The combined organic layer was washed with brine, dried over magnesium sulfate, filtered and concentrated. Purification by silica gel column chromatography using 0 to 50% EtOAc in hexanes gave 4-(4-benzyloxy-6-chloro-2-methyl-3-pyridyl)pyrimidine-2-carbonitrile (93 mg, 87%). ESI-MS m/z calc. 336.07, found 338.3 (M+1)⁺.

### Step 3: 4-[4-benzyloxy-6-(4-tert-butyl-5-chloro-2-methyl-phenyl)-2-methyl-3-pyridyl]pyrimidine-2-carbonitrile

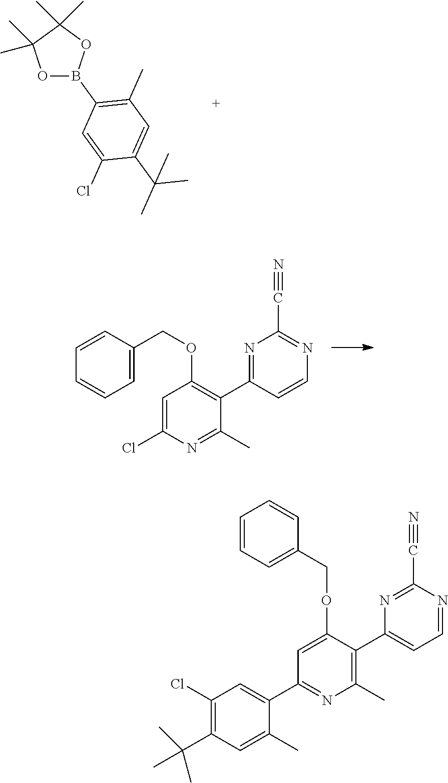 [SMILES: Cc1cc(C(C)(C)C)c(Cl)cc1B1OC(C)(C)C(C)(C)O1 ; Cc1nc(Cl)cc(OCc2ccccc2)c1-c1ccnc(C#N)n1 ; Cc1cc(C(C)(C)C)c(Cl)cc1-c1cc(OCc2ccccc2)c(-c2ccnc(C#N)n2)c(C)n1]

4-[4-benzyloxy-6-(4-tert-butyl-5-chloro-2-methyl-phenyl)-2-methyl-3-pyridyl]pyrimidine-2-carbonitrile was prepared from 2-(4-tert-butyl-5-chloro-2-methyl-phenyl)-4,4,5,5-tetramethyl-1,3,2-dioxaborolane, using procedure analogous to that found in Example 28 (Step 3). ESI-MS m/z calc. 482.18, found 483.5 (M+1)⁺.

### Step 4: 4-[4-benzyloxy-6-(4-tert-butyl-5-chloro-2-methyl-phenyl)-2-methyl-3-pyridyl]pyrimidine-2-carboxamide

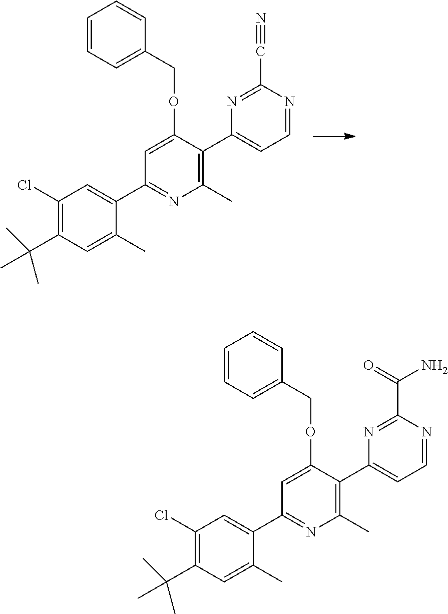 [SMILES: Cc1cc(C(C)(C)C)c(Cl)cc1-c1cc(OCc2ccccc2)c(-c2ccnc(C#N)n2)c(C)n1 ; Cc1cc(C(C)(C)C)c(Cl)cc1-c1cc(OCc2ccccc2)c(-c2ccnc(C(N)=O)n2)c(C)n1]

To a solution of 4-[4-benzyloxy-6-(4-tert-butyl-5-chloro-2-methyl-phenyl)-2-methyl-3-pyridyl]pyrimidine-2-carbonitrile (50 mg, 0.10 nmol) and potassium carbonate (45 mg, 0.32 mmol) in DMSO (1 mL) was added hydrogen peroxide in water (160 μL of 30% w/v, 1.41 mmol). The resulting reaction mixture was stirred at room temperature for 16 hours. The reaction mixture was quenched with water and extracted with EtOAc. The combined organic layer was washed with brine, dried over magnesium sulfate, filtered and concentrated. The crude material was purified via silica gel column chromatography using 0 to 100% EtOAc in hexanes to obtain 4-[4-benzyloxy-6-(4-tert-butyl-5-chloro-2-methyl-phenyl)-2-methyl-3-pyridyl]pyrimidine-2-carboxamide (37 mg, 71%). ESI-MS m/z calc. 500.19, found 501.5 (M+1)⁺.

### Step 5: 4-[6-(4-tert-butyl-5-chloro-2-methyl-phenyl)-2-methyl-4-oxo-1H-pyridin-3-yl]pyrimidine-2-carboxamide (215)

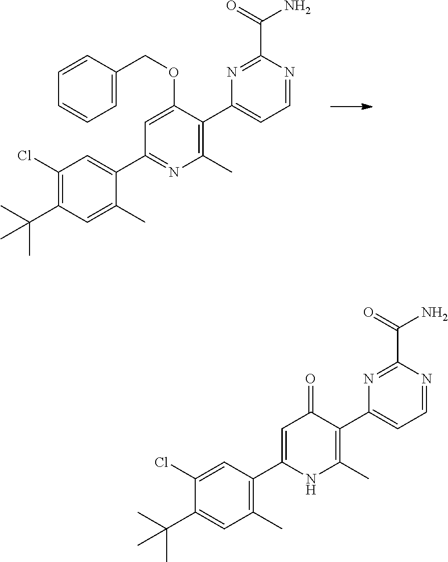 [SMILES: Cc1cc(C(C)(C)C)c(Cl)cc1-c1cc(OCc2ccccc2)c(-c2ccnc(C(N)=O)n2)c(C)n1 ; Cc1cc(C(C)(C)C)c(Cl)cc1-c1cc(=O)c(-c2ccnc(C(N)=O)n2)c(C)[nH]1]

4-[6-(4-tert-butyl-5-chloro-2-methyl-phenyl)-2-methyl-4-oxo-1H-pyridin-3-yl]pyrimidine-2-carboxamide (215) was prepared from 4-[4-benzyloxy-6-(4-tert-butyl-5-chloro-2-methyl-phenyl)-2-methyl-3-pyridyl]pyrimidine-2-carboxamide, using procedure analogous to Example 28 (Step 4). ESI-MS m/z calc. 410.15, found 411.5 (M+1)⁺. ¹H NMR (400 MHz, DMSO-d₆) δ 11.66 (s, 1H), 8.91 (d, J=5.2 Hz, 1H), 8.19-8.10 (m, 1H), 7.81 (d, J=5.1 Hz, 1H), 7.77 (s, 1H), 7.46 (s, 1H), 7.43 (s, 1H), 6.21 (s, 1H), 2.33 (s, 3H), 2.32 (s, 3H), 1.48 (s, 9H).

### Example 35

### 3-[6-(4-tert-butyl-5-chloro-2-methyl-phenyl)-2-methyl-4-oxo-1H-pyridin-3-yl]-1H-pyrazin-2-one (216)

### Step 1: 3-[6-(4-tert-butyl-5-chloro-2-methyl-phenyl)-2-methyl-4-oxo-1H-pyridin-3-yl]-1H-pyrazin-2-one (216)

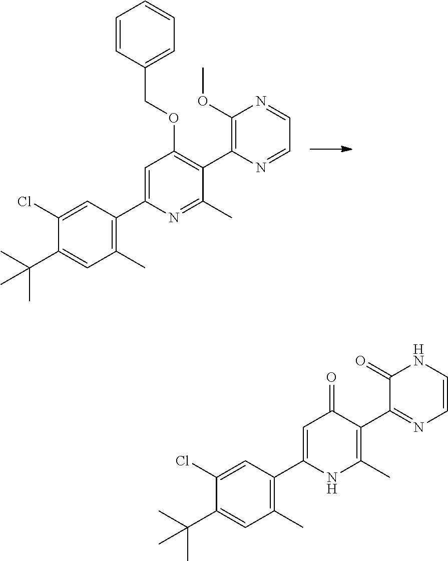 [SMILES: COc1nccnc1-c1c(OCc2ccccc2)cc(-c2cc(Cl)c(C(C)(C)C)cc2C)nc1C ; Cc1cc(C(C)(C)C)c(Cl)cc1-c1cc(=O)c(-c2ncc[nH]c2=O)c(C)[nH]1]

2-[4-benzyloxy-6-(4-tert-butyl-5-chloro-2-methyl-phenyl)-2-methyl-3-pyridyl]-3-methoxy-pyrazine was dissolved in 1,4-dioxane (1 mL) and aqueous HCl (1.5 mL of 1 M, 1.5 mmol) was added and the resulting mixture was stirred at 100° C. overnight. After partial completion, the resulting mixture was diluted with ethyl acetate, basified, washed with a saturated aqueous solution of ammonium chloride and then brine. The organic layer was dried over anhydrous magnesium sulfate, filtered, and concentrated under reduced pressure. Purification by reverse phase HPLC (C₁₈) using 10 to 99% acetonitrile in water (HCl modifier) yielded 3-[6-(4-tert-butyl-5-chloro-2-methyl-phenyl)-2-methyl-4-oxo-1H-pyridin-3-yl]-1H-pyrazin-2-one (216.4 mg, 14%). ESI-MS m/z calc. 397.15, found 398.5 (M+1)⁺. ¹H NMR (400 MHz, CD₃OD) δ 7.68 (d, J=4.0 Hz, 1H), 7.60 (d, J=4.0 Hz, 1H), 7.57-7.53 (m, 2H), 7.09 (s, 1H), 2.49 (s, 3H), 2.36 (s, 3H), 1.53 (s, 9H). ESI-MS m/z calc. 397.15, found 398.5 (M+1)⁺.

### Example 36

### 6-(4-tert-butyl-5-chloro-2-methyl-phenyl)-2-methyl-3-(triazol-1-yl)-1H-pyridin-4-one (217)

### Step 1: 6-(4-tert-butyl-5-chloro-2-methyl-phenyl)-2-methyl-3-(triazol-1-yl)-1H-pyridin-4-one (217)

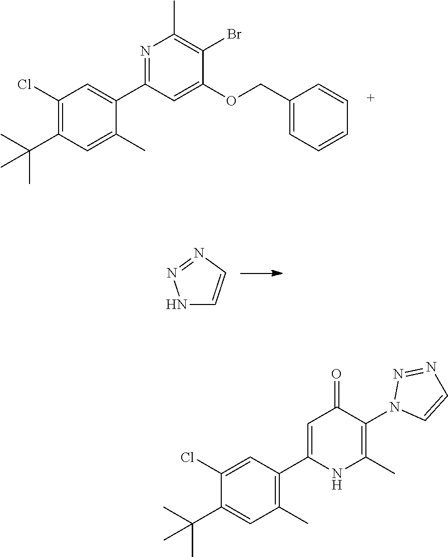 [SMILES: Cc1cc(C(C)(C)C)c(Cl)cc1-c1cc(OCc2ccccc2)c(Br)c(C)n1 ; c1c[nH]nn1 ; Cc1cc(C(C)(C)C)c(Cl)cc1-c1cc(=O)c(-n2ccnn2)c(C)[nH]1]

A microwave vial was charged with 4-benzyloxy-3-bromo-6-(4-tert-butyl-5-chloro-2-methyl-phenyl)-2-methyl-pyridine (45 mg, 0.1 mmol), 1H-triazole (30 μL), CuI (19 mg, 1 mmol), N,N′-dimethylethane-1,2-diamine (30 μL, 0.28 mmol) and DMSO (1 L). The reaction mixture was then degassed for 5 minutes under nitrogen. The tube was sealed and stirred at 150° C. overnight. The reaction mixture was filtered and purified twice by reverse phase HPLC (C₁₈) using 10-99% acetonitrile in water containing 5 nM HCl to yield 6-(4-tert-butyl-5-chloro-2-methyl-phenyl)-2-methyl-3-(triazol-1-yl)-1H-pyridin-4-one (217, 5 mg, 14%). ¹H NMR (400 MHz, CD₃OD) δ 8.23 (d, J=1.1 Hz, 1H), 7.99 (d, J=1.2 Hz, 1H), 7.54 (s, 1H), 7.53 (s, 1H), 6.90 (s, 1H), 2.37 (s, 3H), 2.33 (s, 3H), 1.53 (s, 9H). ESI-MS m/z calc. 356.14, found 357.3 (M+1)⁺.

The following compounds were synthesized using the route analogous to that shown in Example 36, using the appropriate heterocyclyls. Ullmann coupling provided the following compounds. (N,N-dimethylethane-1,2-diamine or cesium carbonate were used as base).

TABLE 9

| | | LC/MS | |
|---|---|---|---|
| | | (m/z calc.); | |
| Cmpd. | | Found | |
| No. | Compound Name | [M + H]⁺ | NMR (shifts in ppm) |
| 218 | 6-(4-tert-butyl-5-chloro-2- | 385.16 | ¹H NMR (400 MHz, CD₃OD) δ 7.55 |
| | methyl-phenyl)-2-methyl- | 386.46 | (s, 1H), 7.54 (s, 1H), 7.13 (s, 1H), |
| | 3-(3-methyl-2-oxo- | | 6.75 (d, J = 3.0 Hz, 1H), 6.61 (d, J = |
| | imidazol-1-yl)-1H-pyridin- | | 3.0 Hz, 1H), 3.36 (s, 3H), 2.50 (s, |
| | 4-one | | 3H), 2.35 (s, 3H), 1.53 (s, 9H). |
| 219 | 6-(4-tert-butyl-5-chloro-2- | 369.16 | ¹H NMR (400 MHz, DMSO-d₆) δ |
| | methyl-phenyl)-2-methyl- | 370.2 | 13.50-12.50 (br s, 1H), 7.61 (br s, |
| | 3-(4-methylpyrazol-1-yl)- | | 1H), 7.53 (br s, 1H), 7.47 (s, 1H), |
| | 1H-pyridin-4-one | | 7.47 (s, 1H), 6.64 (s, 1H), 2.31 (s, |
| | | | 3H), 2.17 (s, 3H), 2.11 (s, 3H), 1.49 |
| | | | (s, 9H). |
| 220 | 6-(4-tert-butyl-5-chloro-2- | 370.16 | ¹H NMR (400 MHz, CD₃OD) δ 8.89 |
| | methyl-phenyl)-2-methyl- | 371.2 | (s, 1H), 7.51 (s, 1H), 7.47 (s, 1H), |
| | 3-(3-methyl-1,2,4-triazol-1- | | 6.69 (s, 1H), 2.51 (s, 3H), 2.36 (s, |
| | yl)-1H-pyridin-4-one | | 3H), 2.35 (s, 3H), 1.52 (s, 9H). |
| 221 | 6-(4-(tert-butyl)-5-chloro- | 356.14 | ¹H NMR (400 MHz, CD₃OD) δ 8.89 |
| | 2-methylphenyl)-2-methyl- | 357.12 | (br s, 1H), 8.38 (br s, 1H), 7.54 (s, |
| | 3-(1H-1,2,4-triazol-1- | | 1H), 7.51 (s, 1H), 6.88 (s, 1H), 2.40 |
| | yl)pyridin-4(1H)-one | | (s, 3H), 2.36 (s, 3H), 1.53 (s, 9H). |

### Example 37

### rel-(S)-5-(6-(4-(tert-butyl)-5-chloro-2-methylphenyl)-2-methyl-4-oxo-1,4-dihydropyridin-3-yl)oxazolidin-2-one (222) & rel-(R)-5-(6-(4-(tert-butyl)-5-chloro-2-methylphenyl)-2-methyl-4-oxo-1,4-dihydropyridin-3-yl)oxazolidin-2-one (223)

### Step 1: SFC Separation: rac-5-[4-benzyloxy-6-(4-tert-butyl-5-chloro-2-methyl-phenyl)-2-methyl-3-pyridyl]oxazolidin-2-one

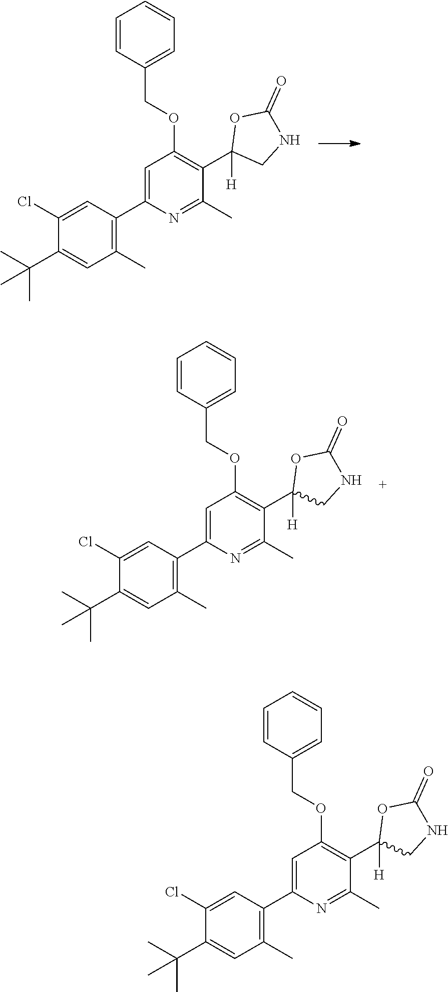 [SMILES: Cc1cc(C(C)(C)C)c(Cl)cc1-c1cc(OCc2ccccc2)c(C2CNC(=O)O2)c(C)n1 ; Cc1cc(C(C)(C)C)c(Cl)cc1-c1cc(OCc2ccccc2)c(C2CNC(=O)O2)c(C)n1 ; Cc1cc(C(C)(C)C)c(Cl)cc1-c1cc(OCc2ccccc2)c(C2CNC(=O)O2)c(C)n1]

rac-5-[4-benzyloxy-6-(4-tert-butyl-5-chloro-2-methyl-phenyl)-2-methyl-3-pyridyl]oxazolidin-2-one was subjected to SFC separation using the following conditions: ChiralPak IG Column 5 μm, (250×21.2 mm) at 40° C., eluent: 28% MeOH (20 mM NH₃), 72% CO₂, flow rate: 70 mL/min, concentration: 26.7 mg/mL in methanol (no modifier), injection volume: 500 μL, pressure: 152 bar, wavelength: 210 nm, 10 minute run. Retention times of enantiomers were determined based on these conditions.
Peak 1 (Enantiomer 1): rel-(S)-5-[4-benzyloxy-6-(4-tert-butyl-5-chloro-2-methyl-phenyl)-2-methyl-3-pyridyl]oxazolidin-2-one. ESI-MS n z calc. 464.18, found 465.28 (M+1)^{m}; Retention time: 6.17 minutes.
Peak 2 (Enantiomer 2): rel-(R) 5-[4-benzyloxy-6-(4-tert-butyl-5-chloro-2-methyl-phenyl)-2-methyl-3-pyridyl]oxazolidin-2-one ESI-MS m/z calc. 464.18, found 465.27 (M+1); Retention time: 7.27 minutes.

### Step 2a: rel-(S)-5-[6-(4-tert-butyl-5-chloro-2-methyl-phenyl)-2-methyl-4-oxo-1H-pyridin-3-yl]oxazolidin-2-one (222)

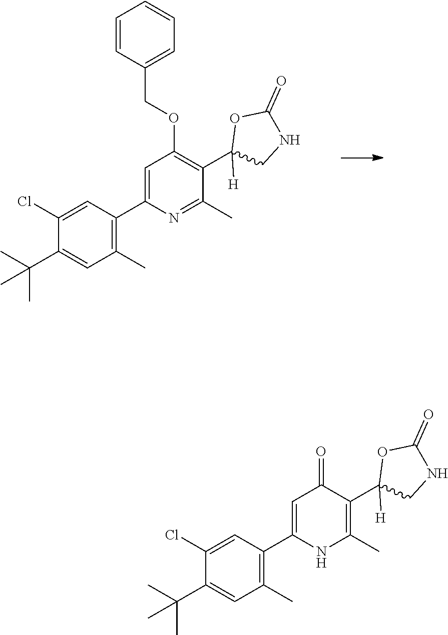 [SMILES: Cc1cc(C(C)(C)C)c(Cl)cc1-c1cc(OCc2ccccc2)c(C2CNC(=O)O2)c(C)n1 ; Cc1cc(C(C)(C)C)c(Cl)cc1-c1cc(=O)c(C2CNC(=O)O2)c(C)[nH]1]

rel-(S)-5-[6-(4-tert-butyl-5-chloro-2-methyl-phenyl)-2-methyl-4-oxo-1H-pyridin-3-yl]oxazolidin-2-one (222) was prepared from rel-(S)-5-[4-benzyloxy-6-(4-tert-butyl-5-chloro-2-methyl-phenyl)-2-methyl-3-pyridyl]oxazolidin-2-one, using a procedure analogous to that found in Example 31 (Step 3). ESI-MS m/z calc. 374.14, found 375.4 (M+1)⁺. ¹H NMR (400 MHz, Methanol-d₄) δ 7.45 (s, 1H), 7.35 (s, 1H), 6.26 (s, 1H), 6.00 (dd, J=9.6, 8.5 Hz, 1H), 3.85 (dd, J=9.7, 8.4 Hz, 1H), 3.74 (t, J=8.4 Hz, 1H), 2.47 (s, 3H), 2.28 (s, 3H), 1.50 (s, 9H).

### Step 2b: rel-(R)-5-[6-(4-tert-butyl-5-chloro-2-methyl-phenyl)-2-methyl-4-oxo-1H-pyridin-3-yl]oxazolidin-2-one (223)

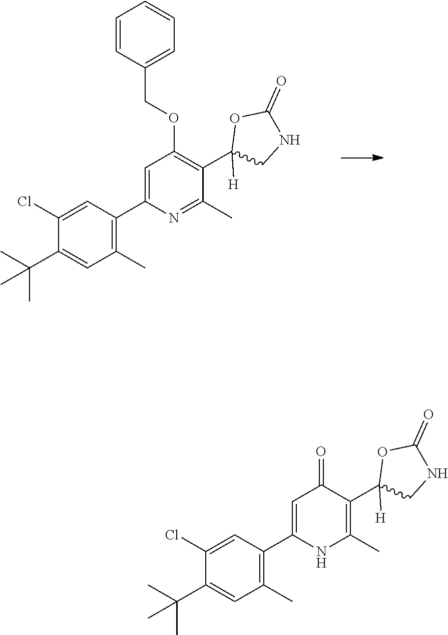 [SMILES: Cc1cc(C(C)(C)C)c(Cl)cc1-c1cc(OCc2ccccc2)c(C2CNC(=O)O2)c(C)n1 ; Cc1cc(C(C)(C)C)c(Cl)cc1-c1cc(=O)c(C2CNC(=O)O2)c(C)[nH]1]

rel-(R)-5-[6-(4-tert-butyl-5-chloro-2-methyl-phenyl)-2-methyl-4-oxo-1H-pyridin-3-yl]oxazolidin-2-one (223) was prepared from rel-(R)-5-[4-benzyloxy-6-(4-tert-butyl-5-chloro-2-methyl-phenyl)-2-methyl-3-pyridyl]oxazolidin-2-one, using procedure analogous to that found in Example 31 (Step 3). ESI-MS m/z calc. 374.14, found 375.4 (M+1)⁺. ¹H NMR (400 MHz, Methanol-d₄) δ 7.45 (s, 1H), 7.35 (s, 1H), 6.26 (s, 1H), 6.00 (dd, J=9.6, 8.5 Hz, 1H), 3.85 (dd, J=9.7, 8.4 Hz, 1H), 3.74 (t, J=8.4 Hz, 1H), 2.47 (s, 3H), 2.28 (s, 3H), 1.50 (s, 9H).

### Example 38

### rel-(S)-4-(6-(4-(tert-butyl)-5-chloro-2-methylphenyl)-2-methyl-4-oxo-1,4-dihydropyridin-3-yl)oxazolidin-2-one (224) & rel-(R)-4-(6-(4-(tert-butyl)-5-chloro-2-methylphenyl)-2-methyl-4-oxo-1,4-dihydropyridin-3-yl)oxazolidin-2-one (225)

### Step 1: SFC Separation: rac-4-[4-benzyloxy-6-(4-tert-butyl-5-chloro-2-methyl-phenyl)-2-methyl-3-pyridyl]oxazolidin-2-one

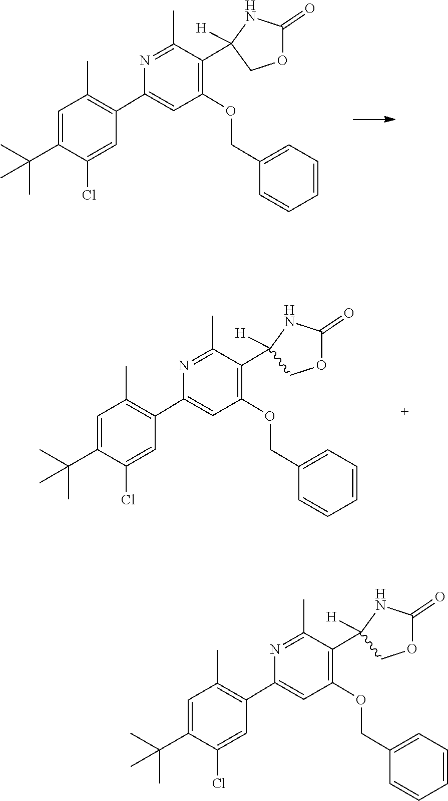 [SMILES: Cc1cc(C(C)(C)C)c(Cl)cc1-c1cc(OCc2ccccc2)c(C2COC(=O)N2)c(C)n1 ; Cc1cc(C(C)(C)C)c(Cl)cc1-c1cc(OCc2ccccc2)c(C2COC(=O)N2)c(C)n1 ; Cc1cc(C(C)(C)C)c(Cl)cc1-c1cc(OCc2ccccc2)c(C2COC(=O)N2)c(C)n1]

rac-4-[4-benzyloxy-6-(4-tert-butyl-5-chloro-2-methyl-phenyl)-2-methyl-3-pyridyl]oxazolidin-2-one was subjected to SFC separation using the following conditions: ChiralPak IC Column 5 m, (250×21.2 mm) at 40° C., using 5 to 80% MeOH (20 mM NH₃), flow rate: variable, concentration: 24 mg/mL in methanol (no modifier), injection volume: 500 μL, pressure: variable, wavelength: 210 nm, 10 minute run. Retention times of enantiomers were determined based on these conditions.
Peak 1 (Enantiomer 1): rel-(S)-4-[4-benzyloxy-6-(4-tert-butyl-5-chloro-2-methyl-phenyl)-2-methyl-3-pyridyl]oxazolidin-2-one (18 mg, 99%). ESI-MS m/z calc. 464.18, found 465.27 (M+1)+; Retention time: 5.43 minutes.
Peak 2 (Enantiomer 2): rel-(R)-4-[4-benzyloxy-6-(4-tert-butyl-5-chloro-2-methyl-phenyl)-2-methyl-3-pyridyl]oxazolidin-2-one (18 mg, 99%) ESI-MS m/z calc. 464.18, found 465.27 (M+1)+; Retention time: 8.52.

### Step 2a: rel-(S)-4-[6-(4-tert-butyl-5-chloro-2-methyl-phenyl)-2-methyl-4-oxo-1H-pyridin-3-yl]oxazolidin-2-one (224)

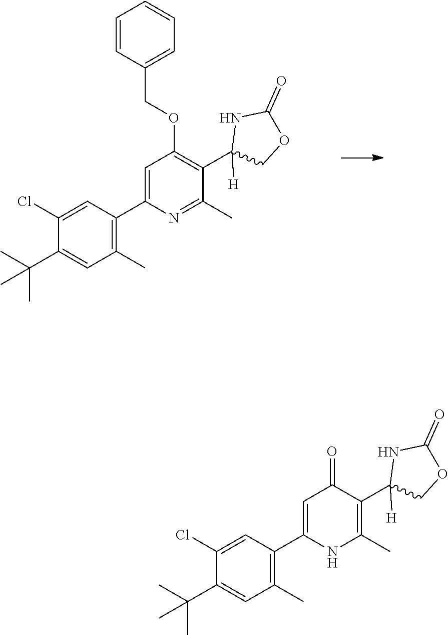 [SMILES: Cc1cc(C(C)(C)C)c(Cl)cc1-c1cc(OCc2ccccc2)c(C2COC(=O)N2)c(C)n1 ; Cc1cc(C(C)(C)C)c(Cl)cc1-c1cc(=O)c(C2COC(=O)N2)c(C)[nH]1]

rel-(S)-4-[6-(4-tert-butyl-5-chloro-2-methyl-phenyl)-2-methyl-4-oxo-1H-pyridin-3-yl]oxazolidin-2-one (224) was prepared from rel-(S)-4-[4-benzyloxy-6-(4-tert-butyl-5-chloro-2-methyl-phenyl)-2-methyl-3-pyridyl]oxazolidin-2-one (Peak 1) using a procedure analogous to that found in Example 31 (Step 3). ESI-MS m/z calc. 374.14, found 375.4 (M+1)⁺. ¹H NMR (400 MHz, Methanol-d₄) δ 7.44 (s, 1H), 7.32 (s, 1H), 6.24 (s, 1H), 5.34 (dd, J=10.0, 6.0 Hz, 1H), 4.69 (dd, J=10.0, 8.3 Hz, 1H), 4.46 (dd, J=8.2, 6.0 Hz, 1H), 2.45 (s, 3H), 2.27 (s, 3H), 1.50 (s, 9H).

### Step 2b: rel-(R)-4-[6-(4-tert-butyl-5-chloro-2-methyl-phenyl)-2-methyl-4-oxo-1H-pyridin-3-yl]oxazolidin-2-one (225)

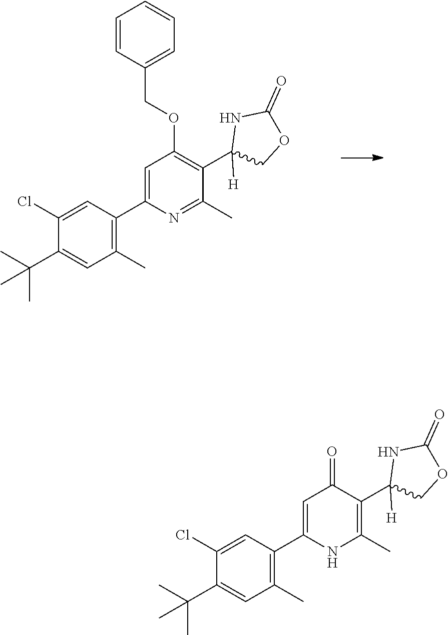 [SMILES: Cc1cc(C(C)(C)C)c(Cl)cc1-c1cc(OCc2ccccc2)c(C2COC(=O)N2)c(C)n1 ; Cc1cc(C(C)(C)C)c(Cl)cc1-c1cc(=O)c(C2COC(=O)N2)c(C)[nH]1]

rel-(R)-4-[6-(4-tert-butyl-5-chloro-2-methyl-phenyl)-2-methyl-4-oxo-1H-pyridin-3-yl]oxazolidin-2-one (225) was prepared from rel-(R)-4-[4-benzyloxy-6-(4-tert-butyl-5-chloro-2-methyl-phenyl)-2-methyl-3-pyridyl]oxazolidin-2-one (Peak 2) using a procedure analogous to that found in Example 31 (Step 3). ESI-MS m/z calc. 374.14, found 375.4 (M+1)⁺. ¹H NMR (400 MHz, Methanol-d₄) δ 7.44 (s, 1H), 7.32 (s, 1H), 6.24 (s, 1H), 5.34 (dd, J=10.0, 6.0 Hz, 1H), 4.69 (dd, J=10.0, 8.3 Hz, 1H), 4.46 (dd, J=8.2, 6.0 Hz, 1H), 2.45 (s, 3H), 2.27 (s, 3H), 1.50 (s, 9H).

### Example 39

### 5-[6-(4-tert-butyl-5-chloro-2-methyl-phenyl)-2-methyl-4-oxo-1H-pyridin-3-yl]isoxazole-3-carboxamide (226)

### Step 1: ethyl 5-[4-benzyloxy-6-(4-tert-butyl-5-chloro-2-methyl-phenyl)-2-methyl-3-pyridyl]isoxazole-3-carboxylate

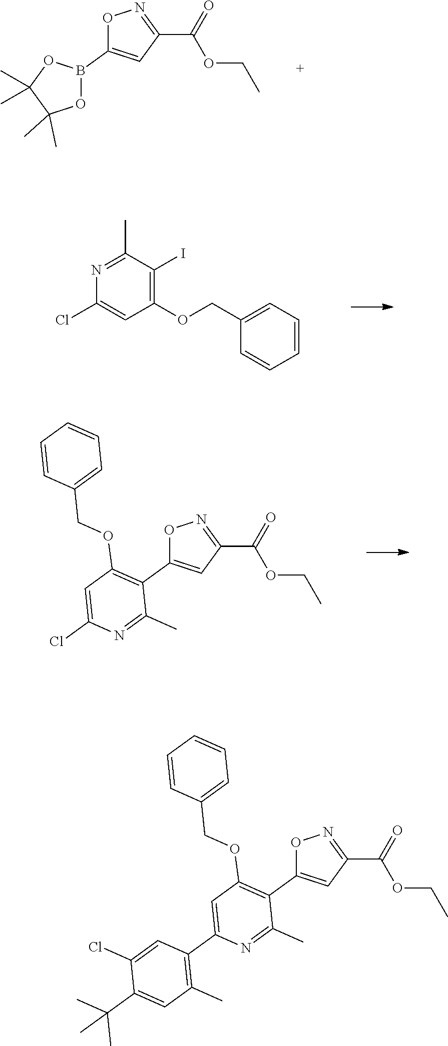

Ethyl 5-[4-benzyloxy-6-(4-tert-butyl-5-chloro-2-methyl-phenyl)-2-methyl-3-pyridyl]isoxazole-3-carboxylate was prepared from 4-benzyloxy-6-chloro-3-iodo-2-methyl-pyridine and ethyl 5-(4,4,5,5-tetramethyl-1,3,2-dioxaborolan-2-yl)isoxazole-3-carboxylate, using a procedure analogous to that found in Example 31 (Step 1 and Step 2). ESI-MS m/z calc. 518.19, found 519.5 (M+1)⁺.

### Step 2: 5-[4-benzyloxy-6-(4-tert-butyl-5-chloro-2-methyl-phenyl)-2-methyl-3-pyridyl]isoxazole-3-carboxamide

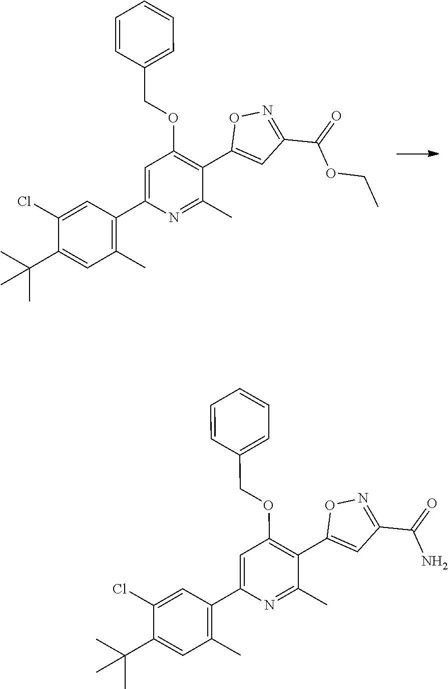

Ethyl 5-[4-benzyloxy-6-(4-tert-butyl-5-chloro-2-methyl-phenyl)-2-methyl-3-pyridyl]isoxazole-3-carboxylate was dissolved in a mixture of 1,4-dioxane (550 L) and MeOH (550 L), followed by NH₄OH (600 L of 30% w/v, 5.13 mmol) and stirred at 45° C. for 35 minutes. The resulting mixture was diluted with ethyl acetate, washed with a saturated aqueous solution of ammonium chloride and then brine. The organic layer was dried over anhydrous magnesium sulfate, filtered, and concentrated under reduced pressure to obtain 5-[4-benzyloxy-6-(4-tert-butyl-5-chloro-2-methyl-phenyl)-2-methyl-3-pyridyl]isoxazole-3-carboxamide. ESI-MS m/z calc. 489.18, found 490.5 (M+1)⁺.

### Step 3: 5-[6-(4-tert-butyl-5-chloro-2-methyl-phenyl)-2-methyl-4-oxo-1H-pyridin-3-yl]isoxazole-3-carboxamide (226)

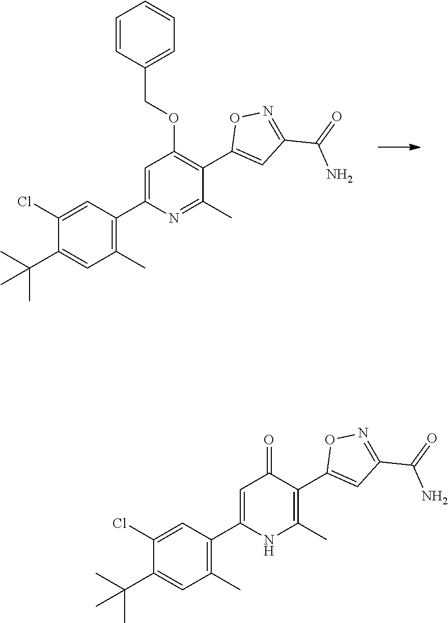 [SMILES: Cc1cc(C(C)(C)C)c(Cl)cc1-c1cc(OCc2ccccc2)c(-c2cc(C(N)=O)no2)c(C)n1 ; Cc1cc(C(C)(C)C)c(Cl)cc1-c1cc(=O)c(-c2cc(C(N)=O)no2)c(C)[nH]1]

5-[6-(4-tert-butyl-5-chloro-2-methyl-phenyl)-2-methyl-4-oxo-1H-pyridin-3-yl]isoxazole-3-carboxamide (226) was prepared from 5-[4-Benzyloxy-6-(4-tert-butyl-5-chloro-2-methyl-phenyl)-2-methyl-3-pyridyl]isoxazole-3-carboxamide, using procedure analogous to that found in Example 31 (Step 3). ESI-MS m/z calc. 399.13, found 400.3 (M+1)⁺. ¹H NMR (400 MHz, CD₃OD) δ 7.47 (s, 1H), 7.41 (s, 1H), 7.21 (s, 1H), 6.39 (s, 1H), 2.53 (s, 3H), 2.33 (s, 3H), 1.51 (s, 9H).

The following compounds were synthesized using a procedure analogous to that shown in Example 39 (Step 1 to Step 3), using the appropriate boronates. Suzuki coupling followed by hydrolysis and deprotection provided the following compound.

TABLE 10

| | | LC/MS | |
|---|---|---|---|
| | | (m/z calc.); | |
| Cmpd. | | Found | |
| No. | Compound Name | [M + H]⁺ | NMR (shifts in ppm) |
| 227 | 5-[6-(4-tert-butyl-5-chloro- | 413.15 | ¹H NMR (400 MHz, CD₃OD) δ 7.48 |
| | 2-methyl-phenyl)-2- | 414.4 | (s, 1H), 7.42 (s, 1H), 6.37 (s, 1H), |
| | methyl-4-oxo-1H-pyridin- | | 2.33 (s, 3H), 2.31 (s, 3H), 2.16 (s, |
| | 3-yl]-4-methyl-oxazole-2- | | 3H), 1.51 (s, 9H). |
| | carboxamide | | |

### Example 40

### 6-(4-tert-butyl-5-chloro-2-methyl-phenyl)-2-methyl-3-[1-(1-methylazetidin-3-yl)pyrazol-4-yl]-1H-pyridin-4-one (Dihydrochloride salt) (228)

### Step 1: 4-benzyloxy-6-(4-tert-butyl-5-chloro-2-methyl-phenyl)-2-methyl-3-[1-(1-methylazetidin-3-yl)pyrazol-4-yl]pyridine

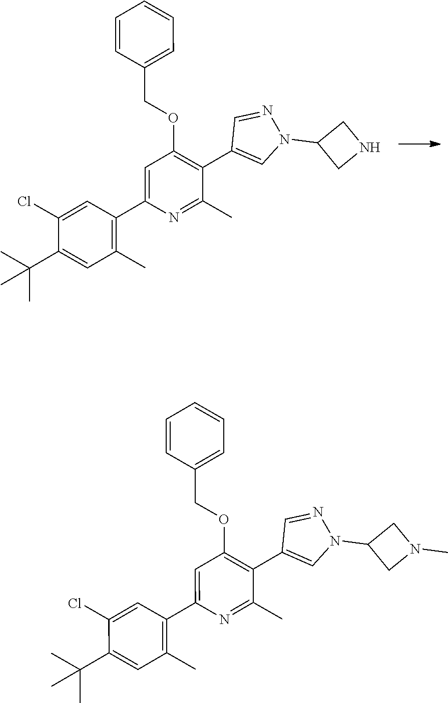 [SMILES: Cc1cc(C(C)(C)C)c(Cl)cc1-c1cc(OCc2ccccc2)c(-c2cnn(C3CNC3)c2)c(C)n1 ; Cc1cc(C(C)(C)C)c(Cl)cc1-c1cc(OCc2ccccc2)c(-c2cnn(C3CN(C)C3)c2)c(C)n1]

To a microwave vial was charged with 3-[1-(azetidin-3-yl)pyrazol-4-yl]-4-benzyloxy-6-(4-tert-butyl-5-chloro-2-methyl-phenyl)-2-methyl-pyridine (117 mg, 0.2335 mmol), formaldehyde (36 mg, 1.2 mmol) and MeOH (5 mL), cyanoboranuide (Sodium salt) (32 mg, 0.51 mmol) was added, followed by the addition of AcOH (30 μL, 0.52 mmol). The tube was sealed and stirred at 25° C. for 24 hours. Additional formaldehyde (36 mg, 1.2 mmol) and cyanoboranuide (Sodium salt) (32 mg, 0.51 mmol) and AcOH (30 μL, 0.53 mmol) were added and the reaction mixture was stirred for 24 hours at room temperature. The resulting mixture was diluted with ethyl acetate, washed with a saturated aqueous solution of ammonium chloride and then brine. The organic layer was dried over anhydrous magnesium sulfate, filtered, and concentrated under reduced pressure. Purification by silica gel column chromatography using 0 to 10% of MeOH in dichloromethane gave 4-benzyloxy-6-(4-tert-butyl-5-chloro-2-methyl-phenyl)-2-methyl-3-[1-(1-methylazetidin-3-yl)pyrazol-4-yl]pyridine. ESI-MS m/z calc. 514.25, found 515.6 (M+1)⁺.

### Step 2: 6-(4-tert-butyl-5-chloro-2-methyl-phenyl)-2-methyl-3-[1-(1-methylazetidin-3-yl)pyrazol-4-yl]-1H-pyridin-4-one (228)

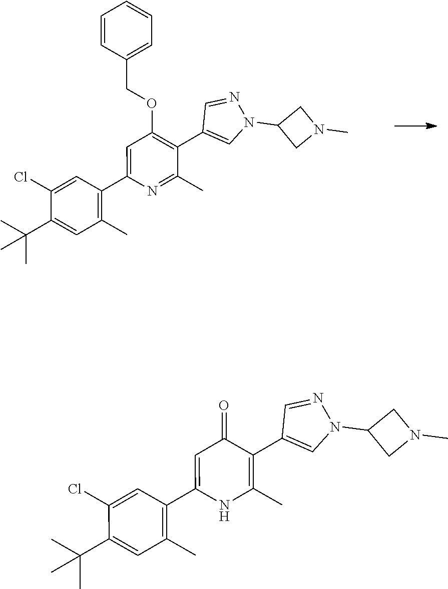

6-(4-tert-butyl-5-chloro-2-methyl-phenyl)-2-methyl-3-[1-(1-methylazetidin-3-yl)pyrazol-4-yl]-1H-pyridin-4-one (Dihydrochloride salt) (228) was prepared from 4-benzyloxy-6-(4-tert-butyl-5-chloro-2-methyl-phenyl)-2-methyl-3-[1-(1-methylazetidin-3-yl)pyrazol-4-yl]pyridine, using a procedure analogous to that found in Example 31 (Step 3). ESI-MS m/z calc. 424.20, found 425.5 (M+1)⁺. ¹H NMR (400 MHz, CD₃OD) δ 8.06 (s, 1H), 7.96 (s, 1H), 7.54 (s, 1H), 7.50 (s, 1H), 6.98 (s, 1H), 5.56-5.44 (m, 1H), 4.97-4.89 (m, 1H), 4.80-4.47 (m, 3H), 3.26-2.98 (m, 3H), 2.63 (s, 3H), 2.34 (s, 3H), 1.53 (s, 9H).

### Example 41

### 6-(4-tert-butyl-5-chloro-2-methyl-phenyl)-2-methyl-3-methylsulfonyl-1H-pyridin-4-one (229)

### Step 1: 6-(4-tert-butyl-5-chloro-2-methyl-phenyl)-2-methyl-3-methylsulfonyl-1H-pyridin-4-one (229)

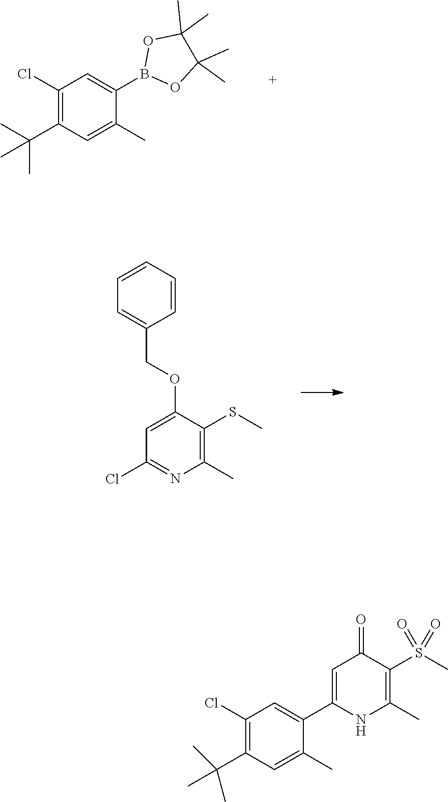 [SMILES: Cc1cc(C(C)(C)C)c(Cl)cc1B1OC(C)(C)C(C)(C)O1 ; CSc1c(OCc2ccccc2)cc(Cl)nc1C ; Cc1cc(C(C)(C)C)c(Cl)cc1-c1cc(=O)c(S(C)(=O)=O)c(C)[nH]1]

6-(4-tert-butyl-5-chloro-2-methyl-phenyl)-2-methyl-3-methylsulfonyl-1H-pyridin-4-one (229, approximately 39.9 mg, 26%) was prepared from 4-benzyloxy-6-(4-tert-butyl-5-chloro-2-methyl-phenyl)-2-methyl-3-methylsulfanyl-pyridine, using a procedure analogous to that found in Example 23 (Step 1 and Step 2). ESI-MS m/z calc. 367.10, found 368.0 (M+1)⁺. ¹H NMR (400 MHz, DMSO-d₆) δ 11.85 (s, 1H), 7.45 (s, 1H), 7.42 (s, 1H), 6.27 (s, 1H), 3.30 (s, 3H), 2.62 (s, 3H), 2.27 (s, 3H), 1.47 (s, 9H).

### Example 42

### 6′-(tert-butyl)-5′-chloro-2′,6-dimethyl-5-(methylsulfonyl)-[2,3′-bipyridin]-4(1H)-one (230)

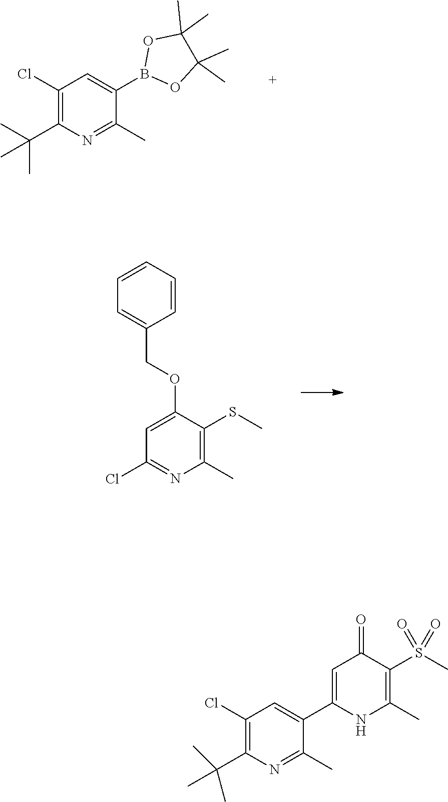 [SMILES: Cc1nc(C(C)(C)C)c(Cl)cc1B1OC(C)(C)C(C)(C)O1 ; CSc1c(OCc2ccccc2)cc(Cl)nc1C ; Cc1nc(C(C)(C)C)c(Cl)cc1-c1cc(=O)c(S(C)(=O)=O)c(C)[nH]1]

6′-(tert-butyl)-5′-chloro-2′,6-dimethyl-5-(methylsulfonyl)-[2,3′-bipyridin]-4(1H)-one (230) was prepared from 4-(benzyloxy)-6′-(tert-butyl)-5′-chloro-2′,6-dimethyl-5-(methylthio)-2,3′-bipyridine, using a procedure analogous to that found in Example 23 (Step 1 and Step 2). ESI-MS m/z calc. 368.10, found 369.0 (M+1)⁺. ¹H NMR (400 MHz, DMSO-d₆) δ 11.81 (s, 1H), 7.86 (s, 1H), 6.37 (s, 1H), 3.29 (s, 3H), 2.63 (s, 3H), 2.44 (s, 3H), 1.48 (s, 9H).

### Example 43

### 6-(4-tert-butyl-5-chloro-2-methyl-phenyl)-3-isopropylsulfonyl-2-methyl-1H-pyridin-4-one (231)

### Step 1: 6-(4-tert-butyl-5-chloro-2-methyl-phenyl)-3-isopropylsulfonyl-2-methyl-1H-pyridin-4-one (231)

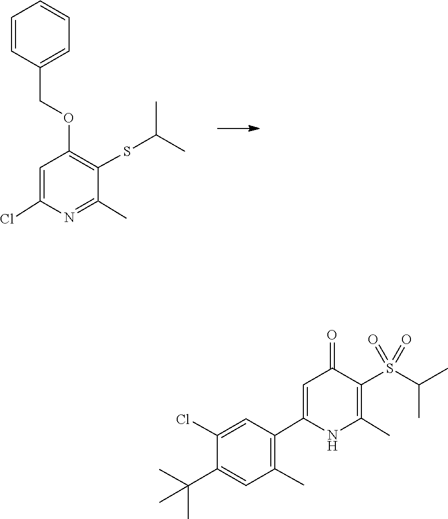 [SMILES: Cc1nc(Cl)cc(OCc2ccccc2)c1SC(C)C ; Cc1cc(C(C)(C)C)c(Cl)cc1-c1cc(=O)c(S(=O)(=O)C(C)C)c(C)[nH]1]

6-(4-tert-butyl-5-chloro-2-methyl-phenyl)-3-isopropylsulfonyl-2-methyl-1H-pyridin-4-one (231) was prepared from 4-benzyloxy-6-chloro-3-isopropylsulfanyl-2-methyl-pyridine, using a procedure analogous to that found in Example 23 (Step 1 and Step 2). ESI-MS m/z calc. 395.13, found 396.0 (M+1)⁺. ¹H NMR (400 MHz, DMSO-d₆) δ 11.73 (s, 1H), 7.44 (s, 1H), 7.42 (s, 1H), 6.19 (s, 1H), 4.07 (h, J=6.8 Hz, 1H), 2.61 (s, 3H), 2.26 (s, 3H), 1.47 (s, 9H), 1.20 (s, 3H), 1.18 (s, 3H).

### Example 44

### 3,5-dibromo-2-(4-tert-butyl-5-chloro-2-methyl-phenyl)-6-methyl-1H-pyridin-4-one (232)

### Step 1: 3,5-dibromo-2-(4-tert-butyl-5-chloro-2-methyl-phenyl)-6-methyl-1H-pyridin-4-one

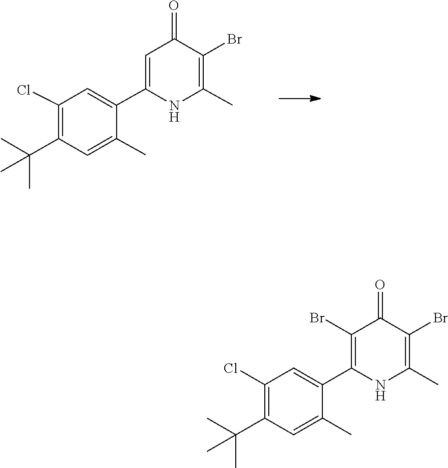 [SMILES: Cc1cc(C(C)(C)C)c(Cl)cc1-c1cc(=O)c(Br)c(C)[nH]1 ; Cc1cc(C(C)(C)C)c(Cl)cc1-c1[nH]c(C)c(Br)c(=O)c1Br]

3,5-dibromo-2-(4-tert-butyl-5-chloro-2-methyl-phenyl)-6-methyl-1H-pyridin-4-one (232) was prepared from 3-bromo-6-(4-tert-butyl-5-chloro-2-methyl-phenyl)-2-methyl-1H-pyridin-4-one, using a procedure analogous to Example 25 (Step 1). ESI-MS m/z calc. 367.03, found 368.19 (M+1)⁺. ¹H NMR (400 MHz, CD₃OD) δ 7.45 (s, 1H), 7.37 (s, 1H), 6.32 (s, 1H), 2.56 (s, 3H), 2.28 (s, 3H), 1.50 (s, 9H).

### Example 45

### 2-(4-tert-butyl-5-chloro-2-methyl-phenyl)-3,5,6-trimethyl-1H-pyridin-4-one (233)

### Step 1: 4-benzyloxy-3-bromo-2-(4-tert-butyl-5-chloro-2-methyl-phenyl)-5,6-dimethyl-pyridine

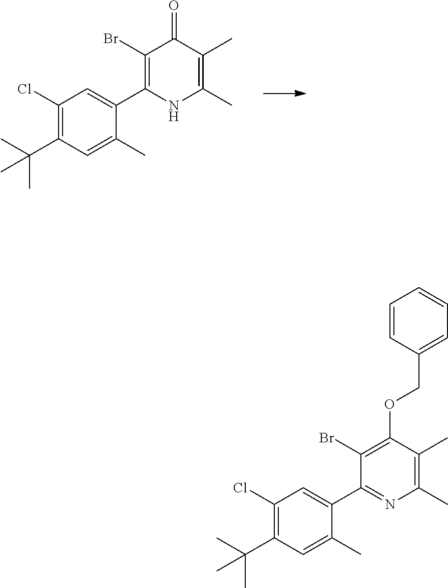 [SMILES: Cc1cc(C(C)(C)C)c(Cl)cc1-c1[nH]c(C)c(C)c(=O)c1Br ; Cc1cc(C(C)(C)C)c(Cl)cc1-c1nc(C)c(C)c(OCc2ccccc2)c1Br]

4-benzyloxy-3-bromo-2-(4-tert-butyl-5-chloro-2-methyl-phenyl)-5,6-dimethyl-pyridine was prepared from 3-bromo-2-(4-tert-butyl-5-chloro-2-methyl-phenyl)-5,6-dimethyl-1H-pyridin-4-one, using a procedure analogous to that found in Example 25 (Step 2). ESI-MS m/z calc. 471.09, found 472.4 (M+1)⁺.

### Step 2: 4-benzyloxy-2-(4-tert-butyl-5-chloro-2-methyl-phenyl)-3,5,6-trimethyl-pyridine

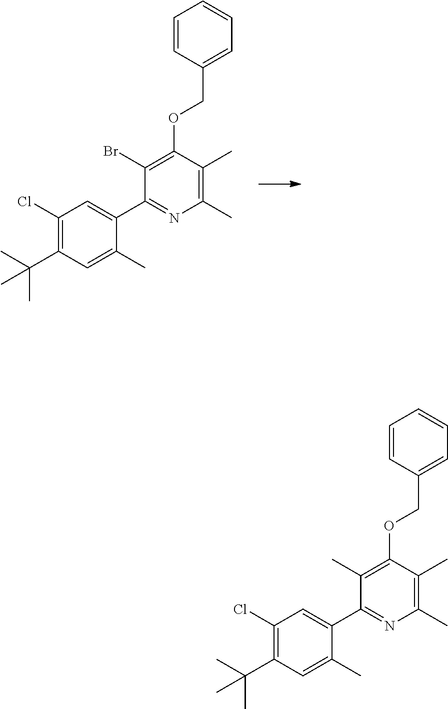 [SMILES: Cc1cc(C(C)(C)C)c(Cl)cc1-c1nc(C)c(C)c(OCc2ccccc2)c1Br ; Cc1cc(C(C)(C)C)c(Cl)cc1-c1nc(C)c(C)c(OCc2ccccc2)c1C]

To a microwave vial was added 4-benzyloxy-3-bromo-2-(4-tert-butyl-5-chloro-2-methyl-phenyl)-5,6-dimethyl-pyridine (26 mg, 0.05 mmol), methylboronic acid (8 mg, 0.13 mmol), potassium carbonate (30 mg, 0.21 mmol), dioxane (350 μL) and water (50 μL). Reaction mixture was degassed with argon for 5 minutes, Pd(dppf)Cl₂-DCM (6 mg, 0.007 mmol) was added, capped and heated to 80° C. under microwave irradiation for 30 minutes. Purification by silica gel column chromatography using 0-100% EtOAc in hexanes gave 4-benzyloxy-2-(4-tert-butyl-5-chloro-2-methyl-phenyl)-3,5,6-trimethyl-pyridine. ESI-MS m/z calc. 407.20, found 408.635 (M+1)⁺.

### Step 3: 2-(4-tert-butyl-5-chloro-2-methyl-phenyl)-3,5,6-trimethyl-1H-pyridin-4-one (233)

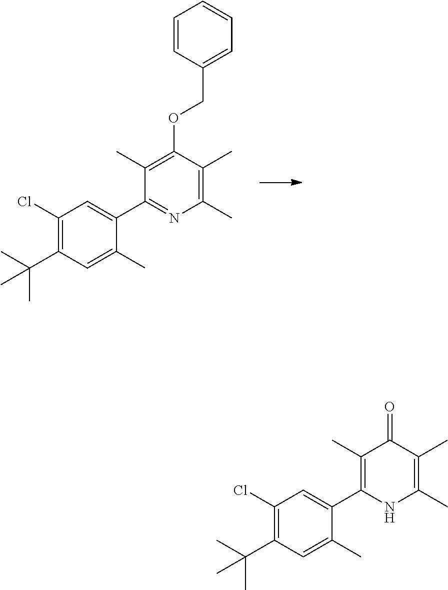 [SMILES: Cc1cc(C(C)(C)C)c(Cl)cc1-c1nc(C)c(C)c(OCc2ccccc2)c1C ; Cc1cc(C(C)(C)C)c(Cl)cc1-c1[nH]c(C)c(C)c(=O)c1C]

2-(4-tert-butyl-5-chloro-2-methyl-phenyl)-3,5,6-trimethyl-1H-pyridin-4-one (233) was prepared from 4-benzyloxy-2-(4-tert-butyl-5-chloro-2-methyl-phenyl)-3,5,6-trimethyl-pyridine, using procedure analogous to that found in Example 4 (Method C, Step 2). ESI-MS m/z calc. 317.15, found 318.33 (M+1)⁺. ¹H NMR (400 MHz, CD₃OD) δ 7.47 (s, 1H), 7.24 (s, 1H), 2.33 (s, 3H), 2.12 (s, 3H), 2.08 (s, 3H), 1.79 (s, 3H), 1.51 (s, 9H).

### Example 46

### 2-(4-tert-butyl-2-methyl-phenyl)-3-(methoxymethyl)-6-methyl-1H-pyridin-4-one (234)

### Step 1: 4-benzyloxy-2-(4-tert-butyl-2-methyl-phenyl)-3-(methoxymethyl)-6-methyl-pyridine

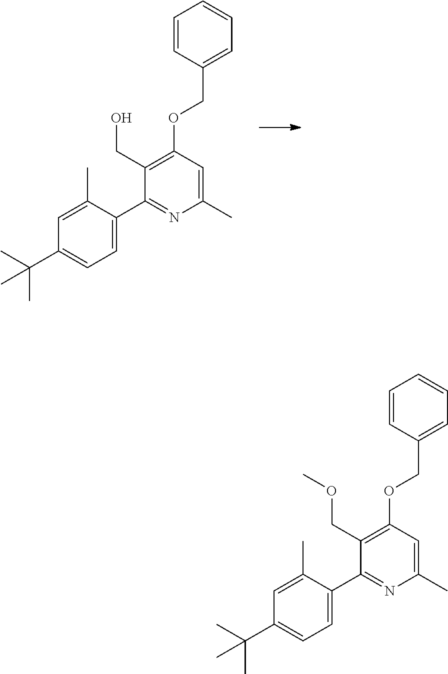 [SMILES: Cc1cc(OCc2ccccc2)c(CO)c(-c2ccc(C(C)(C)C)cc2C)n1 ; COCc1c(OCc2ccccc2)cc(C)nc1-c1ccc(C(C)(C)C)cc1C]

A solution of [4-benzyloxy-2-(4-tert-butyl-2-methyl-phenyl)-6-methyl-3-pyridyl]methanol in DMF (5 mL) at 0° C. was treated with sodium hydride (60% dispersion in mineral oil) (30 mg of 60% w/w, 0.75 mmol) and the mixture was allowed to warm to room temperature over 30 minutes. The reaction was then cooled to 0° C. and iodomethane (80 μL, 1.28 mmol) was added. The reaction was warmed to room temperature and stirred for 2 h. The reaction was then quenched with saturated aqueous ammonium chloride, diluted with water, extracted with EtOAc (3×), washed with water and brine, dried over anhydrous magnesium sulfate, filtered, and concentrated. The crude material was purified by reverse phase preparative HPLC (C₁₈) using 1-60% ACN in water (5 mM HCl), to afford 4-benzyloxy-2-(4-tert-butyl-2-methyl-phenyl)-3-(methoxymethyl)-6-methyl-pyridine (80 mg, 30%) as an off-white solid. ESI-MS m/z calc. 389.23, found 390.57 (M+1)⁺.

### Step 2: 2-(4-tert-butyl-2-methyl-phenyl)-3-(methoxymethyl)-6-methyl-1H-pyridin-4-one (234)

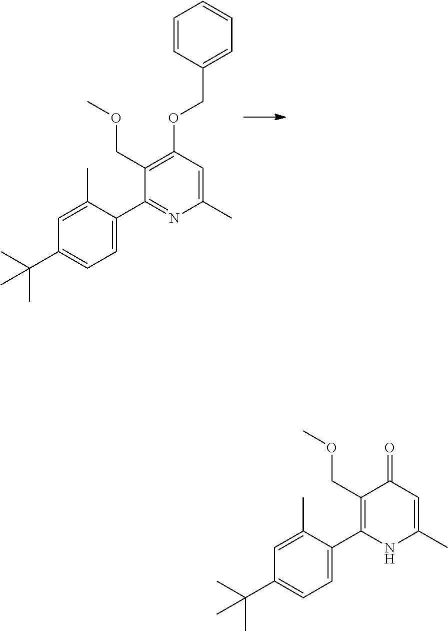 [SMILES: COCc1c(OCc2ccccc2)cc(C)nc1-c1ccc(C(C)(C)C)cc1C ; COCc1c(-c2ccc(C(C)(C)C)cc2C)[nH]c(C)cc1=O]

2-(4-tert-butyl-2-methyl-phenyl)-3-(methoxymethyl)-6-methyl-1H-pyridin-4-one (Hydrochloride salt) (234) was prepared from 4-benzyloxy-2-(4-tert-butyl-2-methyl-phenyl)-3-(methoxymethyl)-6-methyl-pyridine, using procedure analogous to that found in Example 4 (Method C, Step 2). ESI-MS m/z calc. 299.18, found 300.3 (M+1)⁺. ¹H NMR (400 MHz, CD₃OD) δ 7.50-7.47 (m, 1H), 7.46-7.41 (m, 1H), 7.28 (d, J=8.1 Hz, 1H), 7.00 (s, 1H), 4.28 (d, J=10.4 Hz, 1H), 4.09 (d, J=10.4 Hz, 1H), 3.21 (s, 3H), 2.57 (s, 3H), 2.17 (s, 3H), 1.37 (s, 9H).

### Example 47

### 6-(4-tert-butyl-5-chloro-2-methyl-phenyl)-1-oxo-2,3,4,5-tetrahydrothiopyrano[3,2-b]pyridin-8-one (235) & 6-(4-tert-butyl-5-chloro-2-methyl-phenyl)-1,1-dioxo-2,3,4,5-tetrahydrothiopyrano[3,2-b]pyridin-8-one (236)

### Step 1: 8-benzyloxy-6-(4-tert-butyl-5-chloro-2-methyl-phenyl)-3,4-dihydro-2H-thiopyrano[3,2-b]pyridine

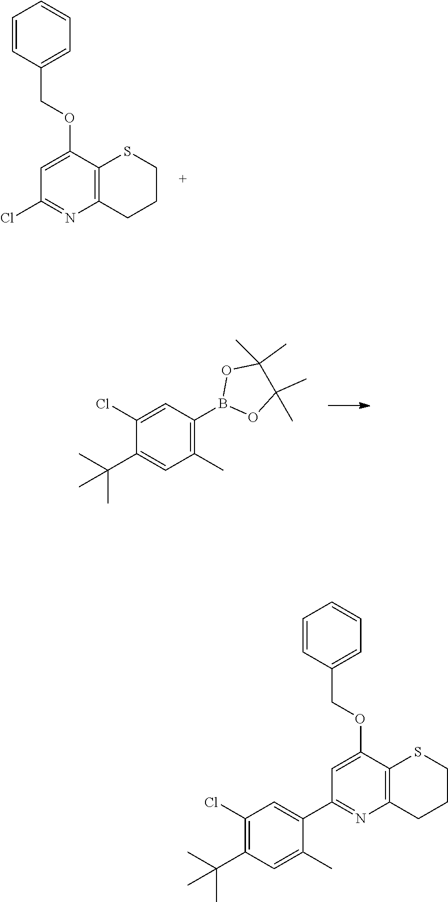 [SMILES: Clc1cc(OCc2ccccc2)c2c(n1)CCCS2 ; Cc1cc(C(C)(C)C)c(Cl)cc1B1OC(C)(C)C(C)(C)O1 ; Cc1cc(C(C)(C)C)c(Cl)cc1-c1cc(OCc2ccccc2)c2c(n1)CCCS2]

8-benzyloxy-6-(4-tert-butyl-5-chloro-2-methyl-phenyl)-3,4-dihydro-2H-thiopyrano[3,2-b]pyridine was prepared from 8-benzyloxy-6-chloro-3,4-dihydro-2H-thiopyrano[3,2-b]pyridine, using procedure analogous to that found in Example 4 (Method C, Step 1). ESI-MS m/z calc. 437.15, found 438.2 (M+1)⁺. ¹H-NMR (400 MHz, CDCl₃) δ 7.44-7.30 (m, 6H), 7.24 (s, 1H), 6.68 (s, 1H), 5.20 (s, 2H), 3.06-3.00 (m, 4H), 2.29-2.23 (m, 2H), 2.20 (s, 3H), 1.46 (s, 9H).

### Step 2: 8-benzyloxy-6-(4-tert-butyl-5-chloro-2-methyl-phenyl)-3,4-dihydro-2H-thiopyrano[3,2-b]pyridine 1-oxide & 8-benzyloxy-6-(4-tert-butyl-5-chloro-2-methyl-phenyl)-3,4-dihydro-2H-thiopyrano[3,2-b]pyridine 1,1-dioxide

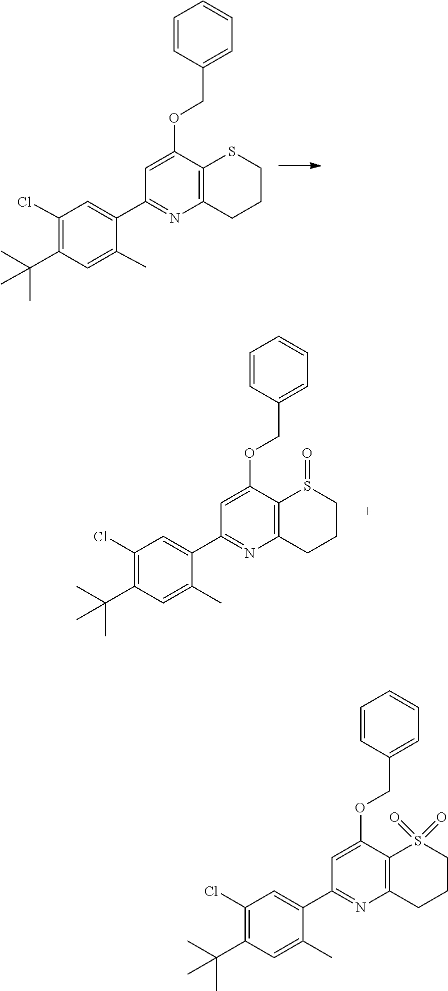 [SMILES: Cc1cc(C(C)(C)C)c(Cl)cc1-c1cc(OCc2ccccc2)c2c(n1)CCCS2 ; Cc1cc(C(C)(C)C)c(Cl)cc1-c1cc(OCc2ccccc2)c2c(n1)CCCS2=O ; Cc1cc(C(C)(C)C)c(Cl)cc1-c1cc(OCc2ccccc2)c2c(n1)CCCS2(=O)=O]

To a solution of 8-benzyloxy-6-(4-tert-butyl-5-chloro-2-methyl-phenyl)-3,4-dihydro-2H-thiopyrano[3,2-b]pyridine (120 mg, 1 mmol) in DCM (4 mL) at 0° C. was added meta-chloroperoxybenzoic acid (63 mg, 75% w/w, 0.27 mmol) and the reaction mixture was stirred for 1 h. DCM (40 mL) was added to the reaction mixture and it was washed with saturated aqueous sodium bicarbonate solution (2×30 mL), water, (30 mL) and brine (30 mL). The organic layer was dried over sodium sulfate, filtered and concentrated under reduced pressure. The crude was purified by silica gel column chromatography using 0 to 100% EtOAc in heptanes to give 8-benzyloxy-6-(4-tert-butyl-5-chloro-2-methyl-phenyl)-3,4-dihydro-2H-thiopyrano[3,2-b]pyridine 1,1-dioxide (16 mg, 13%). (ESI-MS m/z calc. 469.15, found 470.18 (M+1)⁺.) and 8-benzyloxy-6-(4-tert-butyl-5-chloro-2-methyl-phenyl)-3,4-dihydro-2H-thiopyrano[3,2-b]pyridine 1-oxide (100 mg, 86%) (ESI-MS m/z calc. 453.15, found 454.18 (M+1)⁺.).

### Step 3a: 6-(4-tert-butyl-5-chloro-2-methyl-phenyl)-1-oxo-2,3,4,5-tetrahydrothiopyrano[3,2-b]pyridin-8-one (235)

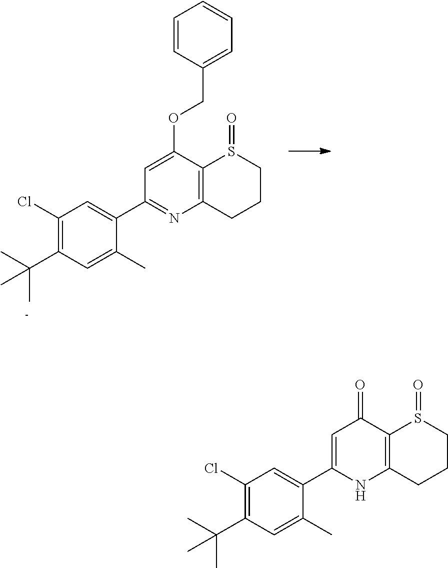

6-(4-tert-butyl-5-chloro-2-methyl-phenyl)-1-oxo-2,3,4,5-tetrahydrothiopyrano[3,2-b]pyridin-8-one (235) was prepared from 8-benzyloxy-6-(4-tert-butyl-5-chloro-2-methyl-phenyl)-3,4-dihydro-2H-thiopyrano[3,2-b]pyridine 1-oxide, using a procedure analogous to that found in Example 4 (Method C, Step 2). ESI-MS m/z calc. 363.11, found 364.16 (M+1)⁺. ¹H-NMR (400 MHz, DMSO-d₆) δ 11.8 (s, 1H), 7.43 (s, 1H), 7.38 (s, 1H), 6.17 (s, 1H), 3.04-3.00 (m, 1H), 2.83-2.59 (m, 3H), 2.34-2.30 (m, 1H), 2.28 (s, 3H), 2.01-1.97 (m, 1H), 1.47 (s, 9H).

### Step 3b: 6-(4-tert-butyl-5-chloro-2-methyl-phenyl)-1,1-dioxo-2,3,4,5-tetrahydrothiopyrano[3,2-b]pyridin-8-one (236)

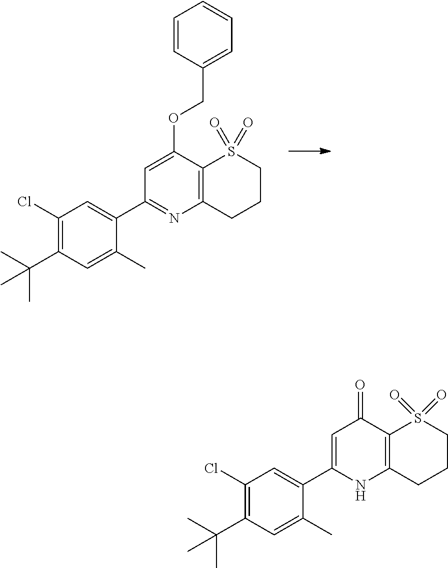 [SMILES: Cc1cc(C(C)(C)C)c(Cl)cc1-c1cc(OCc2ccccc2)c2c(n1)CCCS2(=O)=O ; Cc1cc(C(C)(C)C)c(Cl)cc1-c1cc(=O)c2c([nH]1)CCCS2(=O)=O]

6-(4-tert-butyl-5-chloro-2-methyl-phenyl)-1,1-dioxo-2,3,4,5-tetrahydrothiopyrano[3,2-b]pyridin-8-one (236) was prepared from 8-benzyloxy-6-(4-tert-butyl-5-chloro-2-methyl-phenyl)-3,4-dihydro-2H-thiopyrano[3,2-b]pyridine 1,1-dioxide, using a procedure analogous to that found in Example 4 (Method C, Step 2). ESI-MS m/z calc. 379.1, found 380.16 (M+1)⁺. ¹H-NMR (400 MHz, DMSO-d₆) δ 11.75 (s, 1H), 7.42 (s, 1H), 7.38 (s, 1H), 6.15 (s, 1H), 3.30-3.27 (m, 2H), 2.85 (t, J=6.0 Hz, 2H), 2.28 (s, 3H), 2.22-2.18 (m, 2H), 1.47 (s, 9H).

### Example 48

### rac-6-(4-tert-butyl-5-chloro-2-methyl-phenyl)-1-imino-1-oxo-2,3,4,5-tetrahydrothiopyrano[3,2-b]pyridin-8-one (237)

### Step 1: rac-8-benzyloxy-6-(4-tert-butyl-5-chloro-2-methyl-phenyl)-1-imino-3,4-dihydro-2H-thiopyrano[3,2-b]pyridine 1-oxide

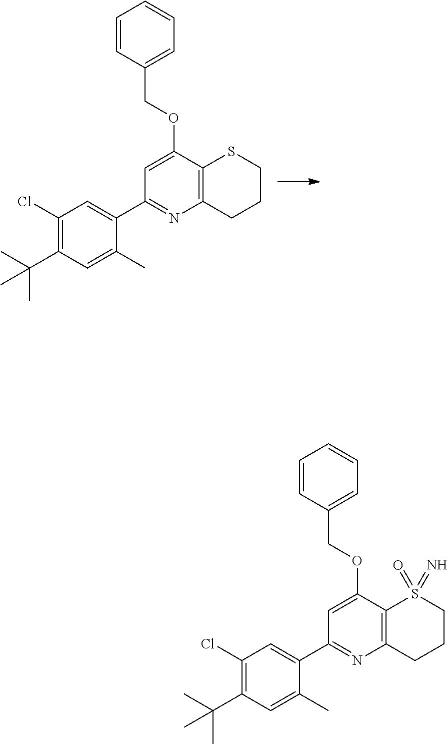 [SMILES: Cc1cc(C(C)(C)C)c(Cl)cc1-c1cc(OCc2ccccc2)c2c(n1)CCCS2 ; Cc1cc(C(C)(C)C)c(Cl)cc1-c1cc(OCc2ccccc2)c2c(n1)CCCS2(=N)=O]

To 8-benzyloxy-6-(4-tert-butyl-5-chloro-2-methyl-phenyl)-3,4-dihydro-2H-thiopyrano[3,2-b]pyridine (722 mg, 1.63 mmol) in DCM (16 mL) and MeOH (16 mL) was added ammonium carbamate (217 mg, 2.78 mmol) and (diacetoxyiodo)benzene (660 mg, 2.05 mmol) and the reaction mixture was heated at 40° C. for 2 h. Additional ammonium carbamate (277.54 mg, 3.55 mmol) and (diacetoxyiodo)benzene (660 mg, 2.05 mmol) were added and the reaction mixture was heated for 2 h at 40° C. The reaction mixture was cooled to room temperature and concentrated in vacuo. Purification by silica gel column chromatography using 20 to 100% EtOAc in heptanes gave rac-8-benzyloxy-6-(4-tert-butyl-5-chloro-2-methyl-phenyl)-1-imino-3,4-dihydro-2H-thiopyrano[3,2-b]pyridine 1-oxide (680 mg, 88%). ESI-MS m/z calc. 468.16, found 469.21 (M+1)⁺. ¹H-NMR (400 MHz, CDCl₃) δ 7.47-7.36 (m, 5H), 7.28 (s, 1H), 7.27 (s, 1H), 6.87 (s, 1H), 5.32 (ABq, J=12.2 Hz, 2H), 3.77 (br s, 1H), 3.58-3.40 (m, 2H), 3.27-3.04 (m, 2H), 2.65-2.52 (m, 1H), 2.46-2.36 (m, 1H), 2.18 (s, 3H), 1.47 (s, 9H).

### Step 2: rac-6-(4-tert-butyl-5-chloro-2-methyl-phenyl)-1-imino-1-oxo-2,3,4,5-tetrahydrothiopyrano[3,2-b]pyridin-8-one (237)

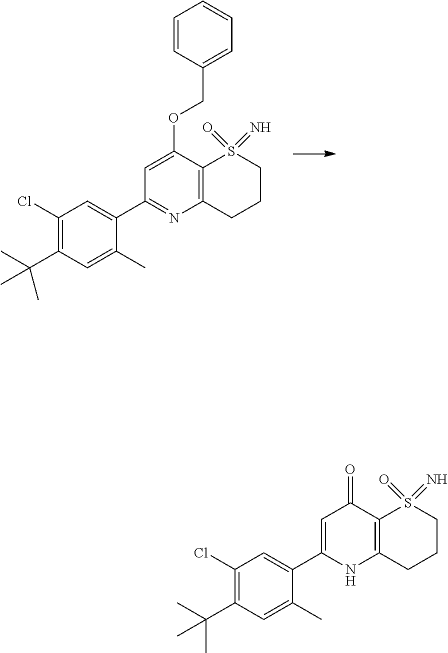 [SMILES: Cc1cc(C(C)(C)C)c(Cl)cc1-c1cc(OCc2ccccc2)c2c(n1)CCCS2(=N)=O ; Cc1cc(C(C)(C)C)c(Cl)cc1-c1cc(=O)c2c([nH]1)CCCS2(=N)=O]

rac-6-(4-tert-butyl-5-chloro-2-methyl-phenyl)-1-imino-1-oxo-2,3,4,5-tetrahydrothiopyrano[3,2-b]pyridin-8-one (237) was prepared from rac-8-benzyloxy-6-(4-tert-butyl-5-chloro-2-methyl-phenyl)-1-imino-3,4-dihydro-2H-thiopyrano[3,2-b]pyridine 1-oxide, using a procedure analogous to that found in Example 4 (Method C, Step 2). ESI-MS m/z calc. 378.12, found 379.17 (M+1)⁺. ¹H-NMR (400 MHz, DMSO-d₆) δ 11.83 (s, 1H), 7.44 (s, 1H), 7.39 (s, 1H), 6.19 (s, 1H). 4.21 (s, 1H), 3.27-3.19 (m, 2H), 2.94-2.82 (m, 2H), 2.27 (s, 3H), 2.22 (td, J=9.6, 4.9 Hz, 2H), 1.47 (s, 9H).

### Example 49

### rel-(S)-6-(4-(tert-butyl)-5-chloro-2-methylphenyl)-1-(methylimino)-1,3,4,5-tetrahydro-114-thiopyrano[3,2-b]pyridin-8(2H)-one 1-oxide (238) & rel-(R)-6-(4-(tert-butyl)-5-chloro-2-methylphenyl)-1-(methylimino)-1,3,4,5-tetrahydro-114-thiopyrano[3,2-b]pyridin-8(2H)-one 1-oxide (239)

### Step 1: SFC Separation: rac-8-benzyloxy-6-(4-tert-butyl-5-chloro-2-methyl-phenyl)-1-imino-3,4-dihydro-2H-thiopyrano[3,2-b]pyridine 1-oxide

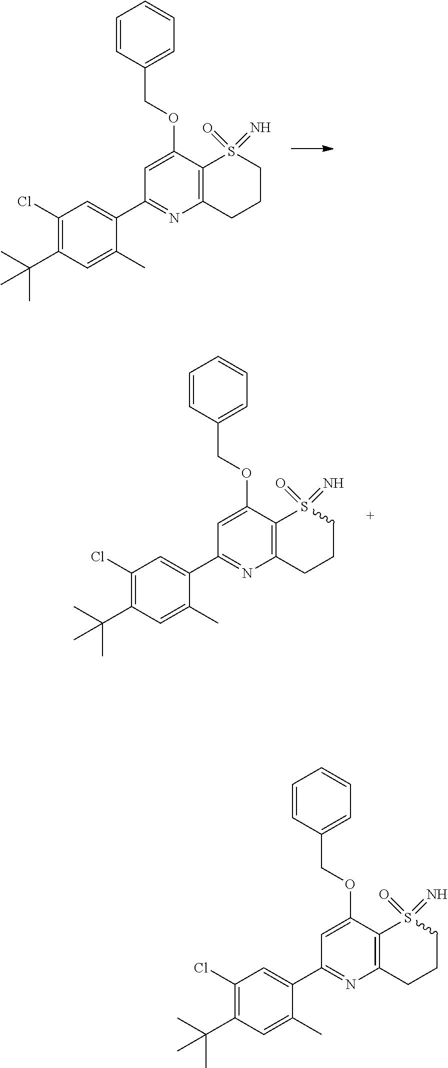

rac-8-benzyloxy-6-(4-tert-butyl-5-chloro-2-methyl-phenyl)-1-imino-3,4-dihydro-2H-thiopyrano[3,2-b]pyridine 1-oxide (434 mg, 0.91 mmol) was subjected to SFC separation using the following conditions: ChiralPak IH Column 5 m, (250×21 mm) at 60° C., eluent: 50% MeOH, 50% CO₂, flow rate: 50 mL/min, injection volume: 1000 μL, pressure: 110 bar, wavelength: 210 nm, 8 minute run. The Retention times were measured using a Biacel I-H analytical column, eluent 5 to 20% Methanol (15 min run).
Peak 1 (Isomer 1): rel-(S)-8-benzyloxy-6-(4-tert-butyl-5-chloro-2-methyl-phenyl)-1-imino-3,4-dihydro-2H-thiopyrano[3,2-b]pyridine 1-oxide (104 mg, 24%). ¹H-NMR (400 MHz, CDCl₃) δ 7.48-7.34 (m, 5H), 7.28 (s, 1H), 7.27 (s, 1H), 6.87 (s, 1H), 5.32 (ABq, J=12.2 Hz, 2H), 3.76 (s, 1H), 3.53 (td, J=13.0, 2.3 Hz, 1H), 3.43 (td, J=6.9, 4.6 Hz, 1H), 3.22 (dt, J=18.3, 4.6 Hz, 1H), 3.13-3.04 (m, 1H), 2.64-2.55 (m, 1H), 2.42-2.38 (m, 1H), 2.18 (s, 3H), 1.47 (s, 9H). ESI-MS m/z calc. 468.16, found 469.11 (M+1)⁺. SFC retention time 9.16 min
Peak 2 (Isomer 2): rel-(R)-8-benzyloxy-6-(4-tert-butyl-5-chloro-2-methyl-phenyl)-1-imino-3,4-dihydro-2H-thiopyrano[3,2-b]pyridine 1-oxide (148 mg, 35%). ¹H-NMR (400 MHz, CDCl₃) δ 7.48-7.34 (m, 5H), 7.28 (s, 1H), 7.27 (s, 1H), 6.87 (s, 1H), 5.32 (ABq, J=12.2 Hz, 2H), 3.76 (s, 1H), 3.53 (td, J=13.0, 2.3 Hz, 1H), 3.43 (td, J=6.9, 4.6 Hz, 1H), 3.22 (dt, J=18.3, 4.6 Hz, 1H), 3.13-3.04 (m, 1H), 2.64-2.55 (m, 1H), 2.42-2.38 (m, 1H), 2.18 (s, 3H), 1.47 (s, 9H). ESI-MS m/z calc. 468.16, found 469.11 (M+1)⁺. Retention time: 0.63 minutes (second eluted isomer). SFC retention time 8.86 min

### Step 2a: rel-(S)-8-benzyloxy-6-(4-tert-butyl-5-chloro-2-methyl-phenyl)-1-methylimino-3,4-dihydro-2H-thiopyrano[3,2-b]pyridine 1-oxide (Isomer 1)

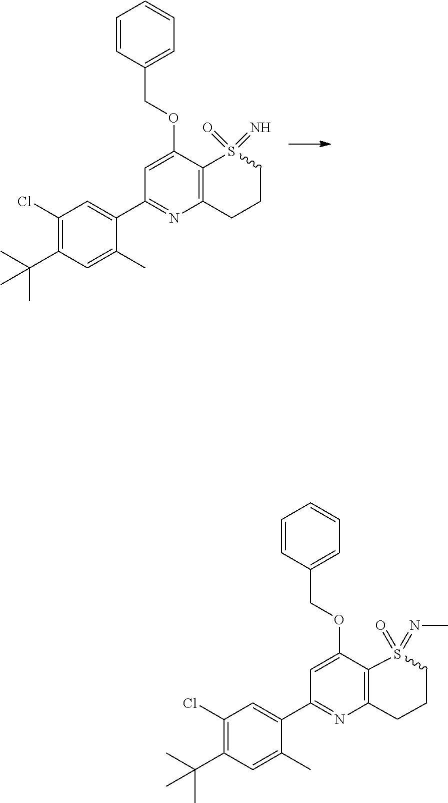

To a solution of rel-(S)-8-benzyloxy-6-(4-tert-butyl-5-chloro-2-methyl-phenyl)-1-imino-3,4-dihydro-2H-thiopyrano[3,2-b]pyridine 1-oxide (98 mg, 0.21 mmol) in DMF (10 mL) at 0° C. under argon was added NaH in oil (17 mg, 60% w/w, 0.43 mmol). The reaction mixture was stirred at this temperature for 20 minutes and MeI (171 mg, 75 μL, 1.20 mmol) was added. The reaction mixture was stirred for an additional for 90 minutes at 0° C. The reaction was quenched by careful addition of saturated ammonium chloride solution (15 mL) and water (15 mL). The aqueous layer was extracted with EtOAc (2×20 mL). The combined organic layer was washed with water (20 mL) and brine (20 mL), dried over sodium sulfate, filtered and concentrated in vacuo. The crude was purified by silica gel column chromatography using 35 to 100% EtOAc in heptanes to obtain rel-(S)-8-benzyloxy-6-(4-tert-butyl-5-chloro-2-methyl-phenyl)-1-methylimino-3,4-dihydro-2H-thiopyrano[3,2-b]pyridine 1-oxide (87 mg, 76%). ESI-MS m/z calc. 482.18, found 483.16 (M+1)⁺.

### Step 2b: rel-(R)-8-benzyloxy-6-(4-tert-butyl-5-chloro-2-methyl-phenyl)-1-methylimino-3,4-dihydro-2H-thiopyrano[3,2-b]pyridine 1-oxide (Isomer 2)

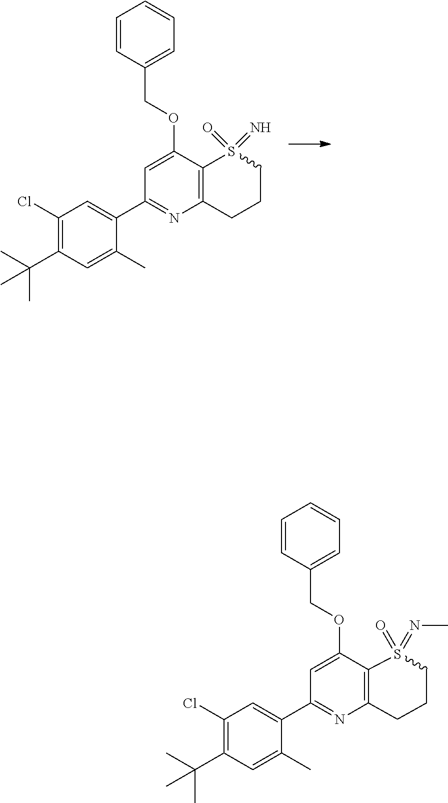

rel-(R)-8-benzyloxy-6-(4-tert-butyl-5-chloro-2-methyl-phenyl)-1-methylimino-3,4-dihydro-2H-thiopyrano[3,2-b]pyridine 1-oxide was prepared from rel-(R)-8-benzyloxy-6-(4-tert-butyl-5-chloro-2-methyl-phenyl)-1-imino-3,4-dihydro-2H-thiopyrano[3,2-b]pyridine 1-oxide, using procedure analogous to that found in Example 48 (Step 2a). ESI-MS m/z calc. 482.18, found 483.16 (M+1)⁺.

### Step 3a: rel-(S)-6-(4-tert-butyl-5-chloro-2-methyl-phenyl)-1-methylimino-1-oxo-2,3,4,5-tetrahydrothiopyrano[3,2-b]pyridin-8-one (238)

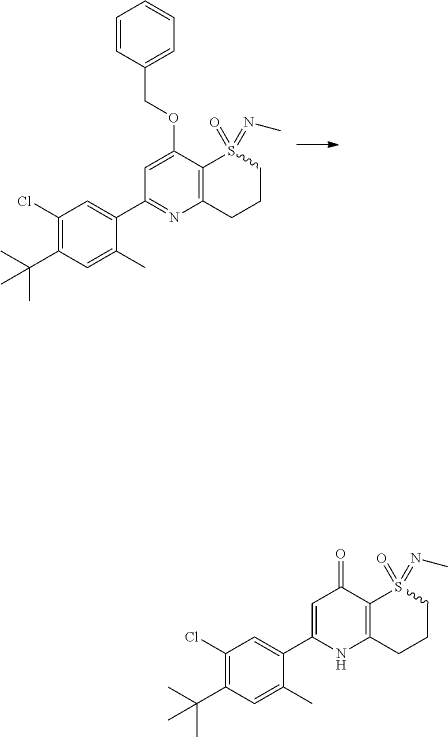

A mixture of rel-(S)-8-benzyloxy-6-(4-tert-butyl-5-chloro-2-methyl-phenyl)-1-methylimino-3,4-dihydro-2H-thiopyrano[3,2-b]pyridine 1-oxide (87 mg, 0.16 mmol) and TFA (1.48 g, 1 mL, 13 mmol) in DCM (10 mL) was heated at 45° C. for 10 h. The volatiles were removed in vacuo and the crude was purified by reverse phase chromatography (C₁₈) using 5 to 20% MeCN in water containing 0.1% formic acid for 1 minute and then changing the gradient to 20 to 60% MeCN in water containing 0.10% formic acid to give rel-(S)-6-(4-tert-butyl-5-chloro-2-methyl-phenyl)-1-methylimino-1-oxo-2,3,4,5-tetrahydrothiopyrano[3,2-b]pyridin-8-one (238, 55 mg, 87%). ESI-MS m/z calc. 392.13, found 393.09 (M+1)⁺. ¹H-NMR (400 MHz, DMSO-d6) δ 7.43 (s, 1H), 7.39 (s, 1H), 6.21 (s, 1H), 3.57-3.50 (m, 1H), 3.43-3.30 (m, 1H), 2.93-2.82 (m, 5H), 2.27 (s, 3H), 2.25-2.17 (m, 2H), 1.46 (s, 9H). SFC analytical method: ChiralPak IH Column 5 m, (250×4.6 mm) at 55° C., eluent: 30% MeOH (20 mM NH₃), 70% CO₂, flow rate: 2.5 mL/min, concentration 0.6 mg/mL (methanol/no modifier), injection volume: 3 μL, pressure: 204 bar, wavelength: 220 nm, 10 minute run. Retention time 8.17 min.

### Step 3b: rel-(R)-6-(4-tert-butyl-5-chloro-2-methyl-phenyl)-1-methylimino-1-oxo-2,3,4,5-tetrahydrothiopyrano[3,2-b]pyridin-8-one (239)

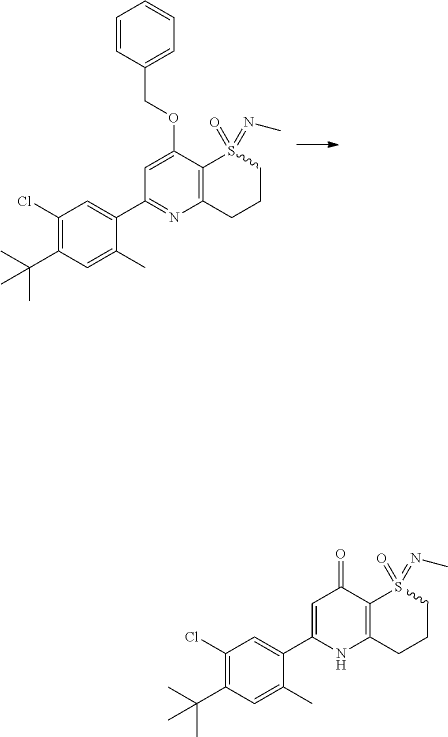

rel-(R)-6-(4-tert-butyl-5-chloro-2-methyl-phenyl)-1-methylimino-1-oxo-2,3,4,5-tetrahydrothiopyrano[3,2-b]pyridin-8-one (239) was prepared from rel-(R)-8-benzyloxy-6-(4-tert-butyl-5-chloro-2-methyl-phenyl)-1-methylimino-3,4-dihydro-2H-thiopyrano[3,2-b]pyridine 1-oxide, using a procedure analogous to Example 48 (Step 3a). ¹H-NMR (400 MHz, DMSO-d6) δ 7.41 (s, 1H), 7.37 (s, 1H), 6.15 (s, 1H), 3.20-3.10 (m, 1H), 3.43-3.30 (m, 1H), 2.89-2.76 (m, 5H), 2.27 (s, 3H), 2.19-2.12 (m, 2H), 1.46 (s, 9H). ESI-MS m/z calc. 392.13, found 393.08 (M+1)⁺. SFC analytical method: ChiralPak IH Column 5 μm, (250×4.6 mm) at 55° C., eluent: 30% MeOH (20 mM NH₃), 70% CO₂, flow rate: 2.5 mL/min, concentration 0.6 mg/mL (methanol/no modifier), injection volume: 3 μL, pressure: 204 bar, wavelength: 220 nm, 10 minute run. Retention time 5.27 min.

### Example 50

### N-acetyl-6-(4-(tert-butyl)-5-chloro-2-methylphenyl)-2-methyl-4-oxo-1,4-dihydropyridine-3-carboxamide (240)

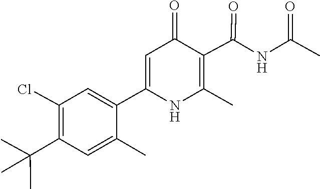 [SMILES: CC(=O)NC(=O)c1c(C)[nH]c(-c2cc(Cl)c(C(C)(C)C)cc2C)cc1=O]

N-acetyl-6-(4-(tert-butyl)-5-chloro-2-methylphenyl)-2-methyl-4-oxo-1,4-dihydropyridine-3-carboxamide (240) was isolated during the synthesis of compound 135. ESI-MS m/z calc. 374.14, found 375.4 (M+1)⁺. ¹H NMR (400 MHz, CD₃OD) δ 7.47 (s, 1H), 7.39 (s, 1H), 6.45 (s, 1H). 2.72 (s, 3H), 2.37 (s, 3H), 2.29 (s, 3H), 1.51 (s, 9H).

### Example 51

### E-VIPR Assay Detecting and Measuring Na_{V} Inhibition Properties

Sodium ion channels are voltage-dependent proteins that can be activated by inducing membrane voltage changes by applying electric fields. The electrical stimulation instrument and methods of use, referred to as E-VIPR, are described in International Publication No. WO 2002/008748 A3 and C.-J. Huang et al. Characterization of voltage-gated sodium channel blockers by electrical stimulation and fluorescence detection of membrane potential, 24 Nature Biotech. 439-46 (2006), both of which are incorporated by reference in their entirety. The instrument comprises a microtiter plate handler, an optical system for exciting the coumarin dye while simultaneously recording the coumarin and oxonol emissions, a waveform generator, a current- or voltage-controlled amplifier, and parallel electrode pairs that are inserted into assay plate wells. Under integrated computer control, this instrument passes user-programmed electrical stimulus protocols to cells within the wells of the microtiter plate.

16-20 hours prior to running the assay on E-VIPR, HEK cells expressing a truncated form of human Na_{V} 1.8 with full channel activity were seeded into microtiter 384-well plates, pre-coated with matrigel, at a density of 25,000 cells per well. 2.5-5% KIR2.1 BacMam virus was added to the final cell suspension before seeding into cell plates. HEK cells were grown in Dulbecco's Modified Eagle's Medium (DMEM) supplemented with 10% FBS (Fetal Bovine Serum, qualified; Sigma #F4135), 1% NEAA (Non-Essential Amino Acids, Gibco #11140), 1% HEPES (Gibco #15630), 1% Pen-Strep (Penicillin-Streptomycin; Gibco #15140) and 5 μg/ml Blasticidin (Gibco #R210-01). Cells were expanded in 5-layer CellSTACK culture chambers or cell culture flasks with vented caps, with 90-95% humidity and 5% CO₂.

### Reagents and Stock Solutions:

100 mg/mL Pluronic F-127 (Sigma #P2443), in dry DMSO
Compound Plates: Corning 384-well Polypropylene Round Bottom #3656
Cell Plates: 384-well tissue culture treated plates (Greiner #781091-2B)
2.5-5% KIR 2.1 Bacmam virus (produced in-house), prepared as described in Section 3.3 of J. A. Fornwald et al., Gene Expression in Mammalian Cells Using BacMam, a Modified Baculovirus System, 1350 Methods in Molecular Biology 95-116 (2016), the entire contents of which are incorporated by reference. The concentration used can be dependent on viral titer of each batch.
5 mM DiSBAC₆(3), a voltage sensitive oxonol acceptor (CAS number 169211-44-3; 5-[3-(1,3-dihexylhexahydro-4,6-dioxo-2-thioxo-5-pyrimidinyl)-2-propen-1-ylidene]-1,3-dihexyldihydro-2-thioxo-4,6(1H,5H)-pyrimidinedione), in dry DMSO. The preparation of DiSBAC₆(3) is analogous to that of DiSBAC₄(3) as described in Voltage Sensing by Fluorescence Resonance Energy Transfer in Single Cells, Gonzalez, J. E. and Tsien, R. Y. (1995) Biophys. J. 69, 1272-1280.
5 mM CC2-DMPE, a commercially available membrane-bound coumarin phospholipid FRET donor (ThermoFisher Scientific catalog number K1017, CAS number 393782-57-5; tetradecanoic acid, 1,1′-[(1R)-1-[8-(6-chloro-7-hydroxy-2-oxo-2H-1-benzopyran-3-yl)-3-hydroxy-3-oxido-8-oxo-2,4-dioxa-7-aza-3-phosphaoct-1-yl]-1,2-ethanediyl]ester) was prepared in dry DMSO. See also, Improved indicators of cell membrane potential that use fluorescence resonance energy transfer, Gonzalez, J. E. and Tsien, R. Y. (1997) Chem. Biol. 4, 269-277.
Voltage Assay Background Suppression Compound (VABSC-1) is prepared in H₂O (89-363 mM, range used to maintain solubility)
Human Serum (HS, Millipore #S1P1-01KL, or Sigma SLBR5469V and SLBR5470V as a 50%/50% mixture, for 25% assay final concentration)

### Bath 1 Buffer:

Sodium Chloride 160 mM (9.35 g/L), Potassium Chloride, 4.5 mM (0.335 g/L), Glucose 10 mM (1.8 g/L), Magnesium Chloride (Anhydrous) 1 mM (0.095 g/L), Calcium Chloride 2 mM (0.222 g/L), HEPES 10 mM (2.38 g/L) in water.

### Na/TMA C1 Bath 1 Buffer:

Sodium Chloride 96 mM (5.61 g/L), Potassium Chloride 4.5 mM (0.335 g/L), Tetramethylammonium (TMA)-C1 64 mM (7.01 g/L), Glucose 10 mM (1.8 g/L), Magnesium Chloride (Anhydrous) 1 mM (0.095 g/L). Calcium Chloride 2 mM (0.222 g/L) HEPES 10 mM (2.38 g/L) in water.

### Hexyl Dye Solution (2× Concentration):

Bath 1 Buffer containing 0.5% 3-cyclodextrin (made fresh prior to each use, Sigma #C4767), 8 M CC2-DMPE and 2 μM DiSBAC₆(3). The solution was made by adding 10% Pluronic F127 stock equal to combined volumes of CC2-DMPE and DiSBAC₆(3). The order of preparation was first mix Pluronic and CC2-DMPE, then add DiSBAC₆(3), then while vortexing add Bath 1/F-Cyclodextrin.
Compound Loading Buffer (2× concentration): Na/TMA C1 Bath1 Buffer containing HS (omitted in experiments run in the absence of human serum (HS))₅₀%, VABSC-1 1 mM, BSA 0.2 mg/ml (in Bath-1), KCl 9 mM, DMSO 0.625%.

### Assay Protocol (7 Key Steps):

1) To reach the final concentration in each well, 375 nL of each compound was pre-spotted (in neat DMSO) into polypropylene compound plates at 240× desired final concentration from an intermediate stock concentration of 0.075 mM, in an 11-point dose response, 3-fold dilution, resulting in a top dose of 300 nM final concentration in the cell plate. Vehicle control (neat DMSO), and positive control (an established Na_{V}1.8 inhibitor, 25 M final in assay in DMSO) were added manually to the outermost columns of each plate respectively. The compound plate was backfilled with 45 μL per well of Compound Loading Buffer resulting in a 240-fold dilution of compound following a 1:1 transfer of compound into the cell plate (see Step 6). Final DMSO concentration for all wells in the assay was 0.625% (0.75% DMSO was supplemented to the Compound Loading Buffer for a final DMSO concentration of 0.625%). This assay dilution protocol was adjusted to enable a higher dose range to be tested in the presence of HS or if the final assay volume was altered.
2) Hexyl Dye Solution was prepared.
3) Cell plates were prepared. On the day of the assay, the media was aspirated, and the cells were washed three times with 80 μL of Bath-1 buffer, maintaining 25 μL residual volume in each well.
4) 25 μL per well of Hexyl Dye Solution was dispensed into the cell plates. The cells were incubated for 20 minutes at room temperature or ambient conditions in darkness.
5) 45 μL per well of Compound Loading Buffer was dispensed into compound plates.
6) The cell plates were washed three times with 80 μL per well of Bath-1 Buffer, leaving 25 μL of residual volume. Then 25 L per well from compound plate was transferred to each cell plate. The mixture was incubated for 30 minutes at room temperature/ambient conditions.
7) The cell plate containing compound was read on E-VIPR using the current-controlled amplifier to deliver stimulation wave pulses using a symmetrical biphasic waveform. The user-programmed electrical stimulus protocols were 1.25-4 Amps and 4 millisecond pulse width (dependent on electrode composition) were delivered at 10 Hz for 10 seconds. A pre-stimulus recording was performed for each well for 0.5 seconds to obtain the un-stimulated intensities baseline. The stimulatory waveform was followed by 0.5 seconds of post-stimulation recording to examine the relaxation to the resting state. All E-VIPR responses were measured at 200 Hz acquisition rate.

### Data Analysis:

Data were analyzed and reported as normalized ratios of emission intensities measured in the 460 nm and 580 nm channels. The response as a function of time was reported as the ratios obtained using the following formula:

$$R ( t ) = \frac{( {intensity}_{460 n m} )}{( {intensity}_{580 n m} )}$$

The data were normalized by calculating the initial (Rᵢ) and final (R_{f}) ratios. These were the average ratio values during part or all of the pre-stimulation period and during sample points during the stimulation period. The fluorescence ratio (R_{f}/Rᵢ) was then calculated and reported as a function of time.

Control responses were obtained by performing assays in the presence of the positive control, and in the absence of pharmacological agents (DMSO vehicle negative control). Responses to the negative (N) and positive (P) controls were calculated as above. The compound antagonist % activity A was then defined as:

$$A = \frac{X - N}{P - N} \times 100$$

where X is the maximum amplitude of the ratio response or number of action potential peaks, at the beginning of the pulse train in the presence of test compound. Using this analysis protocol, dose response curves were plotted and IC₅₀ values were generated for various compounds of the present invention as reported below in Table C.

Compounds having a measured IC₅₀ value less than 0.5 M in the E-VIPR Assay described above include: 2, 76, 81, 85-88, 91-94, 97, 99, 102, 108-138, 140-144, 146-153, 158-161, 165-171, 173, 175-190, 192-196, 198-200, 202, 203, 206-211, and 213-240.

Compounds having a measured IC₅₀ value less than 2 μM and greater than or equal to 0.5 μM in the E-VIPR Assay described above include: 3, 75, 77, 79, 82, 96, 98, 100, 106, 107, 139, 145, 154, 155, 157, 172, 197, 204, 205, and 212.

Compounds having a measured IC₅₀ value less than 5 μM and greater than or equal to 2 M in the E-VIPR Assay described above include: 43, 78, 83, 95, 104, 156, 174, and 201.

Compounds having a measured IC₅₀ value greater than or equal to 5 μM in the E-VIPR Assay described above include: 1, 4-42, 44-74, 80, 84, 89, 90, 101, 103, 105, and 162-164.

Compounds for which an IC₅₀ value was not determined include: 191.

Many modifications and variations of the embodiments described herein may be made without departing from the scope, as is apparent to those skilled in the art. The specific embodiments described herein are offered by way of example only.

1. A compound of formula (I): 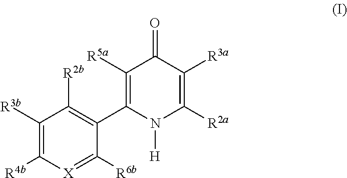
or a pharmaceutically acceptable salt thereof, wherein:
X is CR^{5b} or N;
R^{2a} and R^{3a} are defined as follows:
(i) R^{2a} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂; and
R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂), —C(O)NHC(O)(C₁-C₆ alkyl), —NHS(O)₂CH₃, —S(C₁-C₆ alkyl), C₃-C₇ cycloalkyl, C₆-C₁₀ aryl, 4-10 membered heterocyclyl, 4-10 membered heteroaryl, wherein said heterocyclyl or heteroaryl is optionally substituted with 1-2 R^{a′}; or
(ii) R^{3a} is —S(O)R′, —S(O)₂R′, —S(O)(NH)R′, —S(O)(N(C₁-C₆ alkyl))R′, or —S(O)₂NCH₃R′; and
R^{2a} and R′, together with the atoms to which they are attached, join together to form a 4-7 membered heterocyclyl;
R^{5a} is H, halo, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), —C(O)O(C₁-C₆ haloalkyl), or 4-10 membered heteroaryl, wherein said heteroaryl is optionally substituted with R^{a′};
each R is independently halo, —CN, —OH, oxo, C₁-C₆ alkyl, C₁-C₆ haloalkyl, C₁-C₆ alkoxy, —(C₁-C₆ alkylene)-(C₁-C₆ alkoxy), —C(O)NH₂, —C(O)OH, —S(O)₂(C₁-C₆ alkyl), C₃-C₇ cycloalkyl, 4-10 membered heterocyclyl, or -(4-10 membered heterocyclyl)-(C₁-C₆ alkyl);
R^{2b}, R^{3b}, and R^{4b} are defined as follows:
(i) R^{2b} is H, halo, C₁-C₆ alkyl, C₁-C₆ alkoxy, —OH, —CH₂OH, —OCH₂CH₂OH, —OCH₂CH₂OCH₃, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
R^{3b} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or —(C₁-C₆ haloalkyl)-(C₃-C₆ cycloalkyl), wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
R^{4b} is H, halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, (C₁-C₆ haloalkyl)-(C₃-C₆ cycloalkyl)-, or 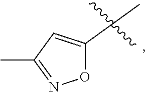 [SMILES: *c1cc(C)no1]
wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; or
(ii) R^{2b} and R^{3b}, together with the carbon atoms to which they are attached, form a ring of formula: 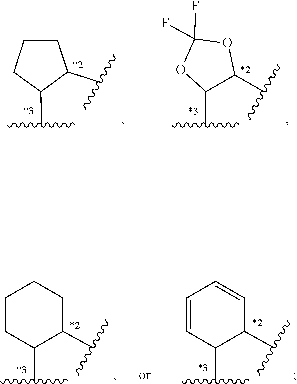 [SMILES: *C1CCCC1* ; *C1OC(F)(F)OC1* ; *C1CCCCC1* ; *C1C=CC=CC1*]
R^{4b} is H, halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or —(C₁-C₆ haloalkyl)-(C₃-C₆ cycloalkyl), wherein said cycloalkyl is optionally substituted with one or more halogen; or
(iii) R^{2b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
R^{3b} and R^{4b}, together with the carbon atoms to which they are attached, form a ring of formula: 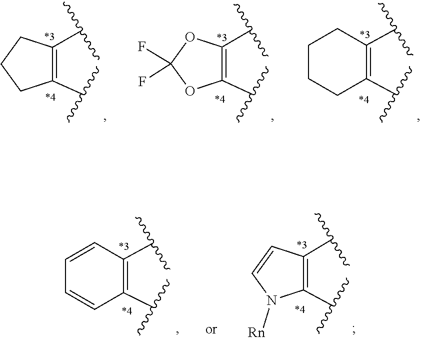
R^{5b} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
R^{6b} is H, halo, C₁-C₆ alkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
Rⁿ is H or C₁-C₆ alkyl,
provided that:
(i) if X is N, then R^{3b} and R^{4b}, together with the carbon atoms to which they are attached, form a ring of formula: 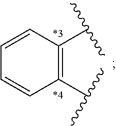 [SMILES: *c1ccccc1*]
and
(ii) if R^{2a} is H, then R^{3a} is halo, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
(iii) if R^{3a} is —C(O)O(C₁-C₂ alkyl), then R^{4b} is H, fluoro, chloro, C₁-C₆ alkyl, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(iv) no more than four of R^{2b}, R^{3b}, R^{4b}, R^{5b}, and R^{6b} are H; and
(v) no more than one of R^{2b}, R^{3b}, R^{4b}, R^{5b}, and R^{6b} is fluoro; and
(vi) no more than one of R^{2b}, R^{3b}, R^{4b}, R^{5b}, and R^{6b} is chloro; and
(vii) if any of R^{2b}, R^{3b}, R^{4b}, R^{5b}, and R^{6b} are halo, then no more than three of R^{2b}, R^{3b}, R^{4b}, R^{5b}, and R^{6b} are H; and
(viii) if R^{2b} or R^{6b} is —OCH₃ and R^{3a} is H, then no more than three of R^{2b}, R^{3b}, R^{4b}, R^{5b}, and R^{6b} are H; and
(ix) if R^{3b} is fluoro, then R^{6b} is H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(x) if R^{5b} is fluoro, then R^{2b} is H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(xi) if R^{3b} or R^{5b} is —CH₃, then no more three of R^{2b}, R^{3b}, R^{4b}, R^{5b}, and R^{6b} are H; and
(xii) if R^{3b} or R^{5b} is C₁ haloalkyl, then no more than three of R^{2b}, R^{3b}, R^{4b}, R^{5b}, and R^{6b} are H; and
(xiii) if R^{3b} or R^{5b} is —OCH₃, then no more than three of R^{2b}, R^{3b}, R^{4b}, R^{5b}, and R^{6b} are H; and
(xiv) if R^{4b} is C₁ haloalkyl, then no more than three of R^{2b}, R^{3b}, R^{5b} and R^{6b} are H; and
(xv) if R^{4b} is —OCH₃, then no more than three of R^{2b}, R^{3b}, R^{5b} and R^{6b} are H; and
(xvi) if R^{4b} is C₁ haloalkoxy, then no more than three of R^{2b}, R^{3b}, R^{5b} and R^{6b} are H; and
(xvii) if R^{4b} is chloro or bromo, then R^{2b} and R^{6b} are each independently H, halo, C₁-C₆ alkyl, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
(xviii) if R^{4b} and R^{3b} are each —OCH₃, then no more than two of R^{2b}, R^{5b}, and R^{6b} are H; and
(xix) if R^{4b} and R^{5b} are each —OCH₃, then no more than two of R^{2b}, R^{3b}, and R^{6b} are H.

2. The compound of claim 1, wherein:
R^{2a} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
R^{5a} is H, C₁-C₆ alkyl, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl).

3. (canceled)

4. The compound of claim 1, wherein X is CR^{5b} and R^{5b} is H, halo, C₁-C₆ alkyl, C₁-C₆ haloalkyl, C₁-C₆ alkoxy, or —C(O)(C₁-C₆ alkyl).

5. (canceled)

6. The compound of claim 1, wherein:
R^{2a} is H, C₁-C₆ alkyl, C₁-C₆ haloalkyl, C₁-C₆ alkoxy, or —N(C₁-C₆ alkyl)₂;
R^{2b} is H, halo, —CN, C₁-C₆ alkyl, C₁-C₆ alkoxy, —NH₂, —N(C₁-C₆ alkyl), —(C₁-C₆ alkylene)-OH, —(C₁-C₆ alkylene)-NH₂, —C(O)(C₁-C₆ alkyl), —C(O)NH₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂), —C(O)NHC(O)CH₃, —NHS(O)₂CH₃, —S(C₁-C₆ alkyl), C₃-C₇ cycloalkyl, C₆-C₁₀ aryl, 4-10 membered heterocyclyl, 4-10 membered heteroaryl, wherein said heterocyclyl or heretoaryl is optionally substituted with 1-2 R_{a′}, wherein each R^{a′} is independently F, Cl, —CN, —OH, oxo, —CH₃, —CH₂CH₃, —CH(CH₃)₂, —CHF₂, —CF₃, —OCH₃, —CH₂OCH₃, —C(O)NH₂, —C(O)OH, —S(O)₂CH₂, cyclopropyl, azetidinyl, or 1-methylazetidinyl.

7-11. (canceled)

12. The compound of claim 1, wherein
(i) R^{3a} is —S(O)₂—(C₁-C₆ alkyl), —S(O)(NH)(C₁-C₆ alkyl), —NHS(O)₂CH₃, —S(C₁-C₆ alkyl); or
(ii) R^{3a} is —S(O)R′, —S(O)₂R′, —S(O)(NH)R′, —S(O)(NCH₃)R′, or —S(O)₂NCH₃R′; and
R^{2a} and R′, together with the atoms to which they are attached, join together to form a 6-membered heterocyclyl.

13. The compound of claim 1, wherein R^{5a} is H, halo, C₁-C₆ alkyl, C₁-C₆ alkoxy, —CH₂OH, —CH₂O(C₁-C₆ alkyl), —C(O)O(C₁-C₆ alkyl), 4-10 membered heteroaryl, wherein said heteroaryl is optionally substituted with R^{a′}; and
R^{a′} is —CH₃, —CHF₂, or —CH₂OCH₃.

14-16. (canceled)

17. The compound of claim 1, wherein:
R^{2b} is H, halo, C₁-C₆ alkyl, C₁-C₆ alkoxy, —OH, —CH₂OH, —OCH₂CH₂OH, or —OCH₂CH₂OCH₃; and/or
R^{3b} is H, halo, C₁-C₆ alkyl, C₁-C₆ haloalkyl, or C₁-C₆ alkoxy; and/or
R^{4b} is H, halo, C₁-C₆ alkyl, C₁-C₆ haloalkyl, C₁-C₆ alkoxy, C₁-C₆ haloalkoxy, C₃-C₆ cycloalkyl, (C₁-C₆ alkyl)-(C₃-C₆ cycloalkyl)-, (C₁-C₆ haloalkyl)-(C₃-C₆ cycloalkyl)-, or 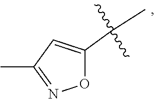 [SMILES: *c1cc(C)no1]
wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen.

18-42. (canceled)

43. The compound of claim 1, wherein the compound is of formula (I-C-1): 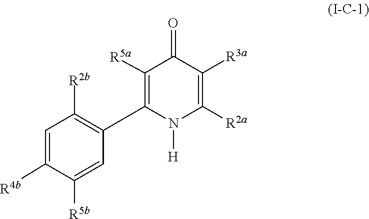
or a pharmaceutically acceptable salt thereof, wherein:
R^{2b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
R^{4b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
R^{5b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen,
provided that:
(i) if R^{2a} is H, then R^{3a} is halo, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂); and
(ii) no more than one of R^{2b}, R^{4b}, and R^{5b} is chloro.

44-45. (canceled)

46. The compound of claim 43,
or a pharmaceutically acceptable salt thereof, wherein:
R^{2a} is H, C₁-C₆ alkyl, C₁-C₆ haloalkyl, C₁-C₆ alkoxy, C₁-C₆ haloalkoxy, —N(C₁-C₆ alkyl)₂, —N(C₁-C₆ alkyl)(C₁-C₆ haloalkyl), or —N(C₁-C₆ haloalkyl)₂;
R^{3a} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), or —NHC(O)CH(CH₃)(NH₂);
R^{5a} is H, C₁-C₆ alkyl, C₁-C₆ haloalkyl, C₁-C₆ alkoxy, C₁-C₆ haloalkoxy; —CH₂OH, —CH₂O(C₁-C₆ alkyl), —CH₂O(C₁-C₆ haloalkyl), —C(O)O(C₁-C₆ alkyl), or —C(O)O(C₁-C₆ haloalkyl);
R^{2b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen;
R^{4b} is halo, C₂-C₆ alkyl, C₁-C₆ haloalkyl, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
R^{5b} is halo, C₁-C₆ alkyl, C₁-C₆ haloalkyl, C₁-C₆ alkoxy, C₁-C₆ haloalkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen,
provided that:
(i) if R^{2a} is H, then R^{3a} is halo, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃)(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂); and
(ii) no more than one of R^{2b}, R^{4b}, and R^{5b} is chloro.

47-63. (canceled)

64. A compound of;
(i) formula (II): 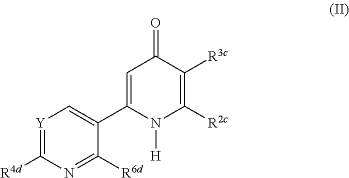
or a pharmaceutically acceptable salt thereof, wherein:
Y is CR^{3d} or N;
R^{2c} is C₁-C₆ alkyl;
R^{3c} is H, halo, C₁-C₆ alkyl, —S(O)₂(C₁-C₆ alkyl), —S(O)(NH)(C₁-C₆ alkyl), or 4-10 membered heteroaryl, wherein said heteroaryl is optionally substituted with C₁-C₆ alkyl;
R^{3d} is halo, C₁-C₆ alkyl, or C₁-C₆ haloalkyl;
R^{4d} is H, C₁-C₆ alkyl, or C₁-C₆ haloalkyl; and
R^{6d} is H, C₁-C₆ alkyl, or C₁-C₆ alkoxy, or
(ii) formula (III): 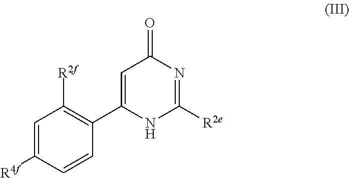
or a pharmaceutically acceptable salt thereof, wherein:
R^{2e} is C₁-C₆ alkyl;
R^{2f} is C₁-C₆ alkyl; and
R^{4f} is —C(CH₃)₃; or
(iii) formula (IV): 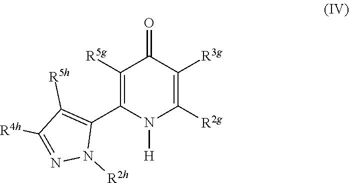
or a pharmaceutically acceptable salt thereof, wherein:
R^{2g} and R^{3g} are defined as follows:
(i) R^{2g} is C₁-C₆ alkyl; and
R^{3g} is H, halo, —CN, C₁-C₆ alkyl, C₁-C₆ haloalkyl, C₁-C₆ alkoxy, C₁-C₆ haloalkoxy, —OH, —NH₂, —NH(C₁-C₆ alkyl), —N(C₁-C₆ alkyl), —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)O(C₁-C₆ alkyl), —S(O)₂—(C₁-C₆ alkyl), —S(O)(NH)(C₁-C₆ alkyl), —CH₂C(O)NH₂, —CH₂C(O)NH(C₁-C₆ alkyl), —CH₂C(O)N(C₁-C₆ alkyl)₂, —NHC(O)(C₁-C₆ alkyl), —CH₂NHC(O)CH(CH₃(NH₂), —CH₂N(CH₃)C(O)CH(CH₃)(N(CH₃)₂), —CH₂CH₂NHC(O)CH(CH₃)(NH₂), —NHC(O)CH(CH₃)(NH₂), —C(O)NHC(O)(C₁-C₆ alkyl), —NHS(O)₂CH₃, —S(C₁-C₆ alkyl), C₃-C₇ cycloalkyl, C₆-C₁₀ aryl, 4-10 membered heterocyclyl, 4-10 membered heteroaryl, wherein said heterocyclyl or heteroaryl is optionally substituted with 1-2 R^{a′};
(ii) R^{3g} is —S(O)R′, —S(O)₂R′, —S(O)(NH)R′, —S(O)(N(C₁-C₆ alkyl))R′, or —S(O)₂NCH₃R′; and
R^{2g} and R′, together with the atoms to which they are attached, join together to form a 4-7 membered heterocyclyl;
R^{5g} is H, halo, C₁-C₆ haloalkyl, C₁-C₆ alkoxy, C₁-C₆ haloalkoxy, —CH₂OH, —CH₂O(C₁-C₆ alkyl), —CH₂O(C₁-C₆ haloalkyl), —C(O)O(C₁-C₆ alkyl), —C(O)O(C₁-C₆ haloalkyl), or 4-10 membered heteroaryl, wherein said heteroaryl is optionally substituted with R^{g′};
each R^{g′} is independently halo, —CN, —OH, oxo, C₁-C₆ alkyl, C₁-C₆ haloalkyl, C₁-C₆ alkoxy, —(C₁-C₆ alkylene)-(C₁-C₆ alkoxy), —C(O)NH₂, —C(O)OH, —S(O)₂(C₁-C₆ alkyl), C₃-C₇ cycloalkyl, 4-10 membered heterocycyl, or -(4-10 membered heterocyclyl)-(C₁-C₆ alkyl);
R^{2h} is H, C₁-C₆ alkyl, C₁-C₆ alkoxy, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one ore more halogen;
R^{4h} and R^{5h} are defined as follows:
(i) R^{4h} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or —(C₁-C₆ haloalkyl)-(C₃-C₆ cycloalkyl), wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; and
R^{5h} is H, halo, C₁-C₆ alkyl, C₁-C₆ haloalkyl, C₁-C₆ alkoxy, —OH, —CH₂OH, —C(O)(C₁-C₆ alkyl), —C(O)(C₁-C₆ haloalkyl), C₃-C₆ cycloalkyl, (C₁-C₆ alkyl)-(C₃-C₆ cycloalkyl)-, or —(C₁-C₆ haloalkyl)-(C₃-C₆ cycloalkyl), wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen; or
(ii) R^{4h} and R^{5h}, together with the C atoms to which they are attached, join together to form a C₅-C₆ cycloalkyl, wherein said cycloalkyl is optionally substituted with 1-2 C₁-C₆ alkyl.

65-79. (canceled)

80. A compound selected from: 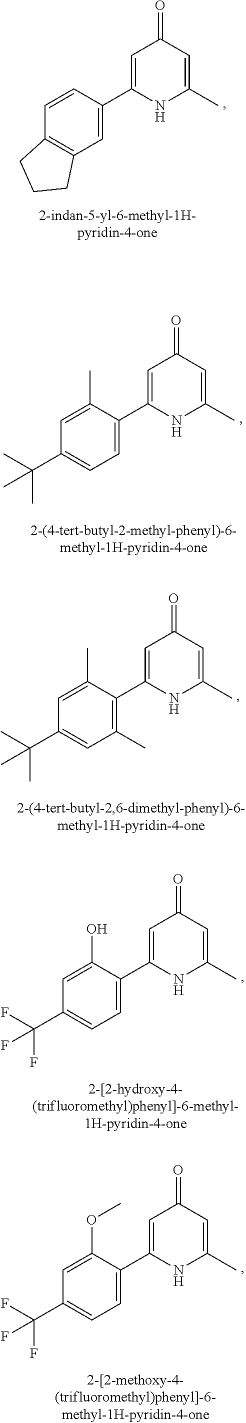 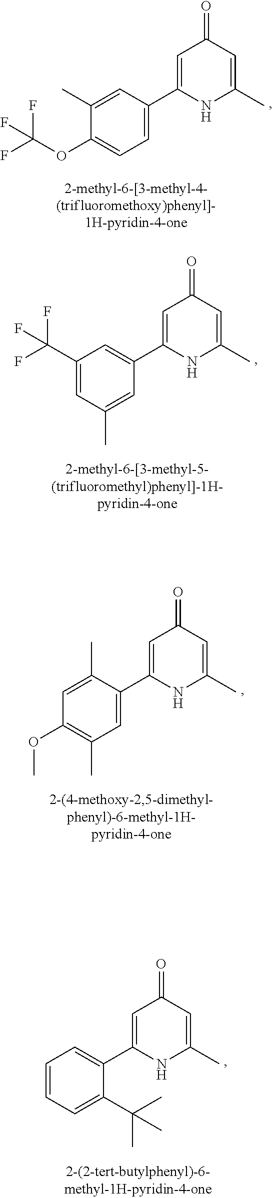 [SMILES: Cc1cc(=O)cc(-c2ccc(OC(F)(F)F)c(C)c2)[nH]1 ; Cc1cc(-c2cc(=O)cc(C)[nH]2)cc(C(F)(F)F)c1 ; COc1cc(C)c(-c2cc(=O)cc(C)[nH]2)cc1C ; Cc1cc(=O)cc(-c2ccccc2C(C)(C)C)[nH]1] 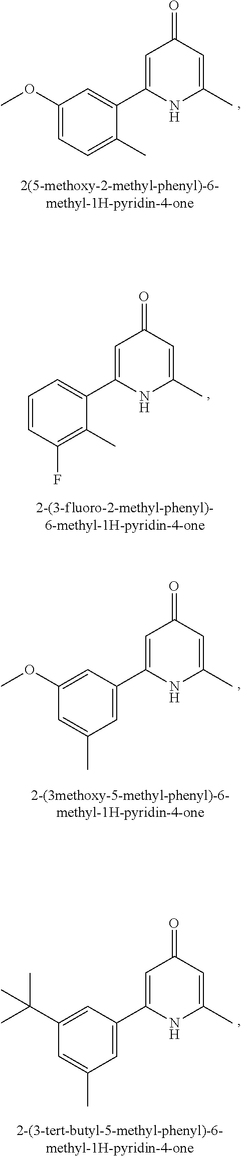 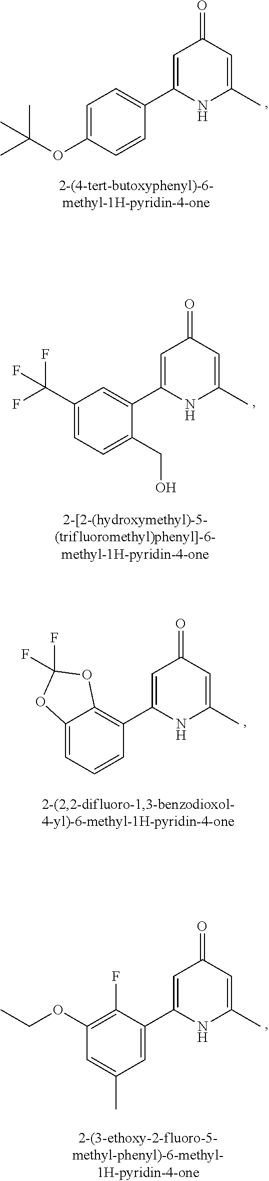 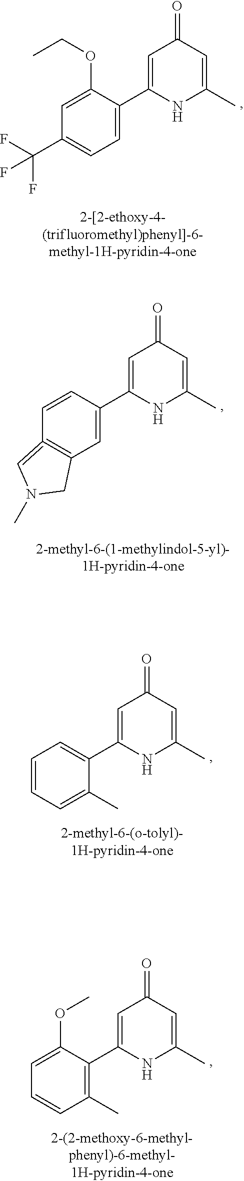 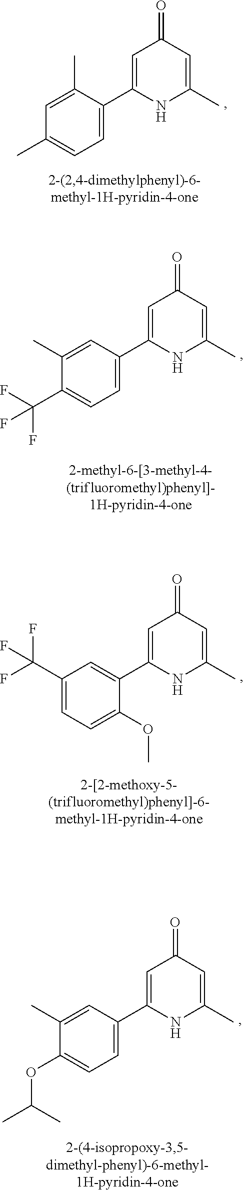 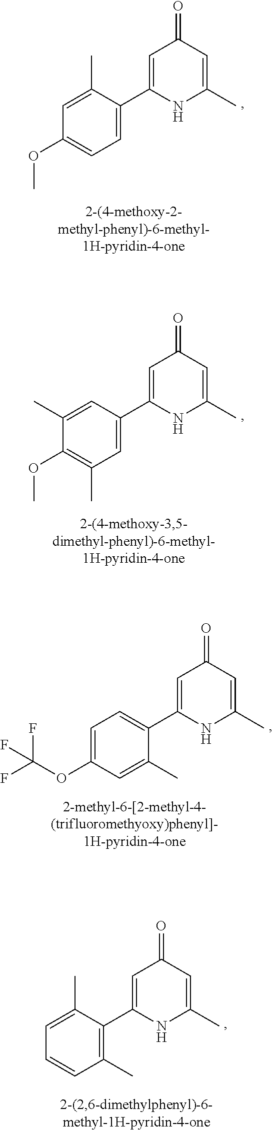 [SMILES: COc1ccc(-c2cc(=O)cc(C)[nH]2)c(C)c1 ; COc1c(C)cc(-c2cc(=O)cc(C)[nH]2)cc1C ; Cc1cc(=O)cc(-c2ccc(OC(F)(F)F)cc2C)[nH]1 ; Cc1cc(=O)cc(-c2c(C)cccc2C)[nH]1] 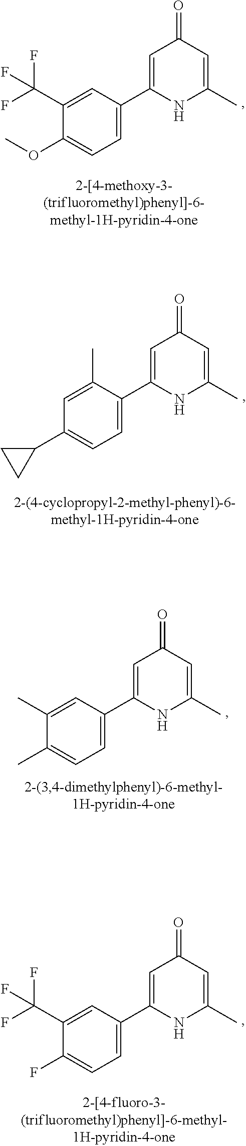 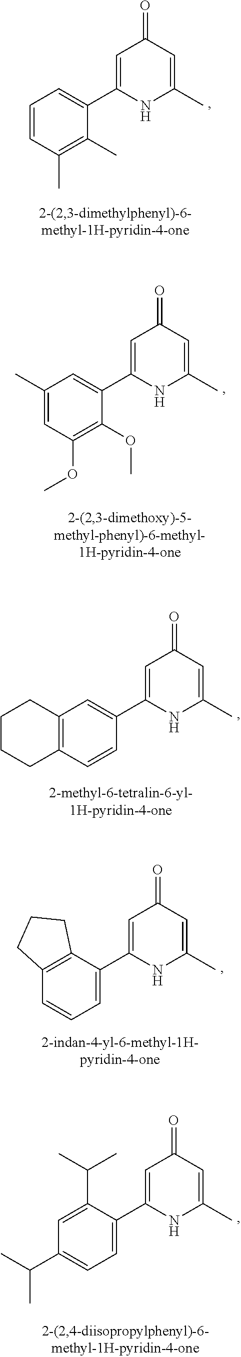 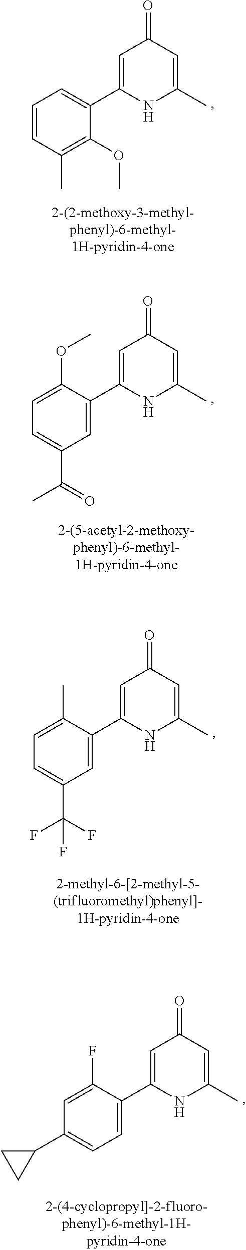 [SMILES: COc1c(C)cccc1-c1cc(=O)cc(C)[nH]1 ; COc1ccc(C(C)=O)cc1-c1cc(=O)cc(C)[nH]1 ; Cc1cc(=O)cc(-c2cc(C(F)(F)F)ccc2C)[nH]1 ; Cc1cc(=O)cc(-c2ccc(C3CC3)cc2F)[nH]1] 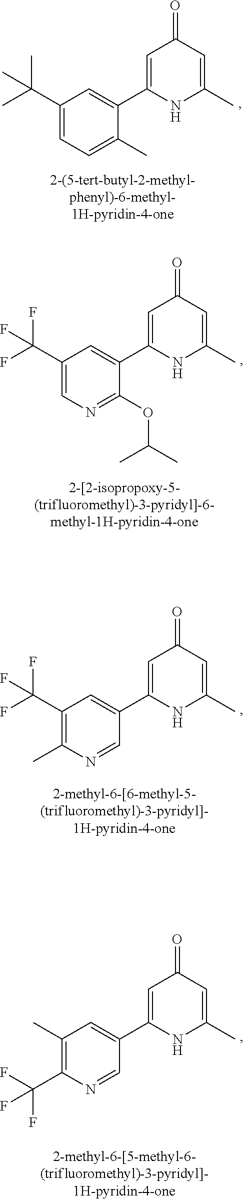 [SMILES: Cc1cc(=O)cc(-c2cc(C(C)(C)C)ccc2C)[nH]1 ; Cc1cc(=O)cc(-c2cc(C(F)(F)F)cnc2OC(C)C)[nH]1 ; Cc1cc(=O)cc(-c2cnc(C)c(C(F)(F)F)c2)[nH]1 ; Cc1cc(=O)cc(-c2cnc(C(F)(F)F)c(C)c2)[nH]1] 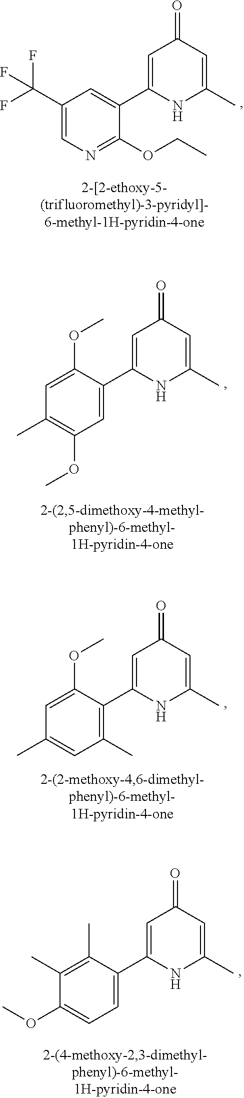 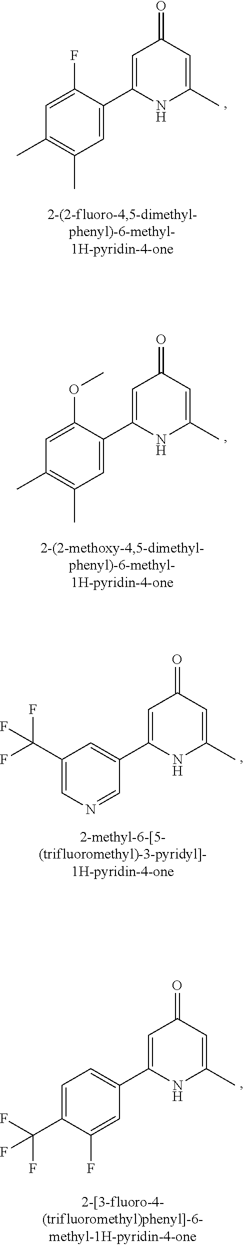 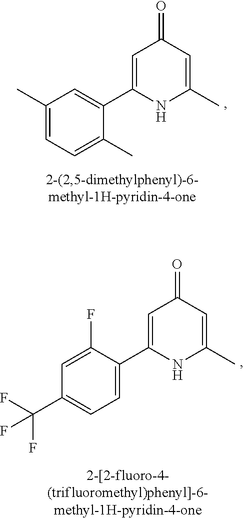 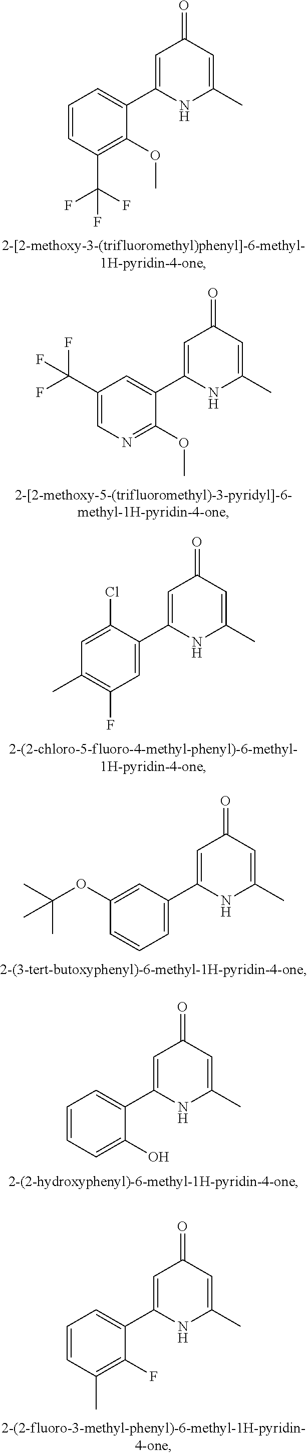 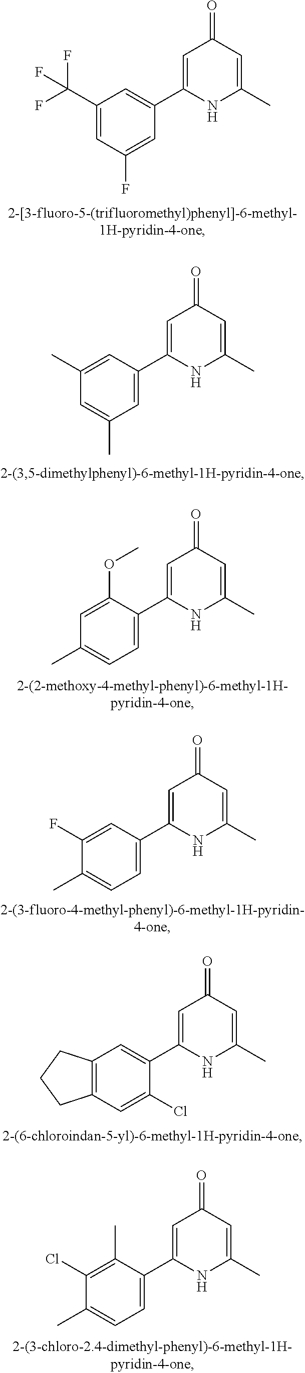 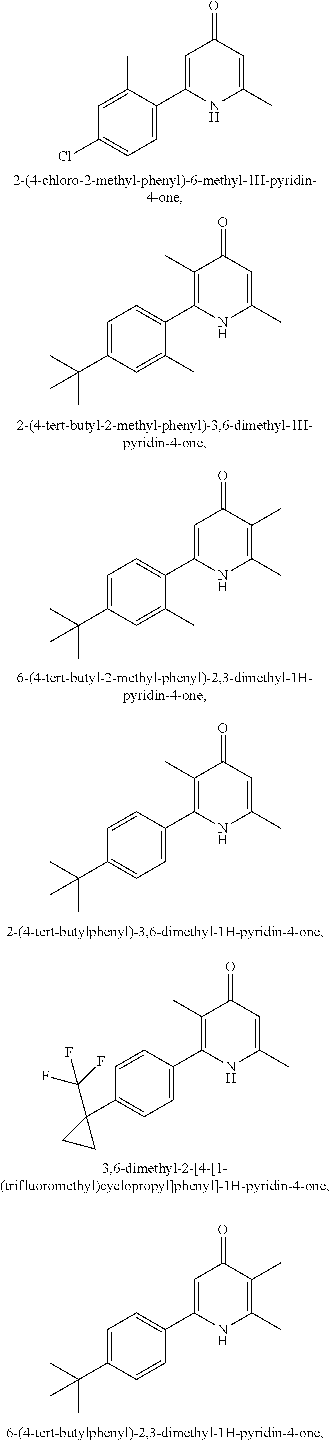 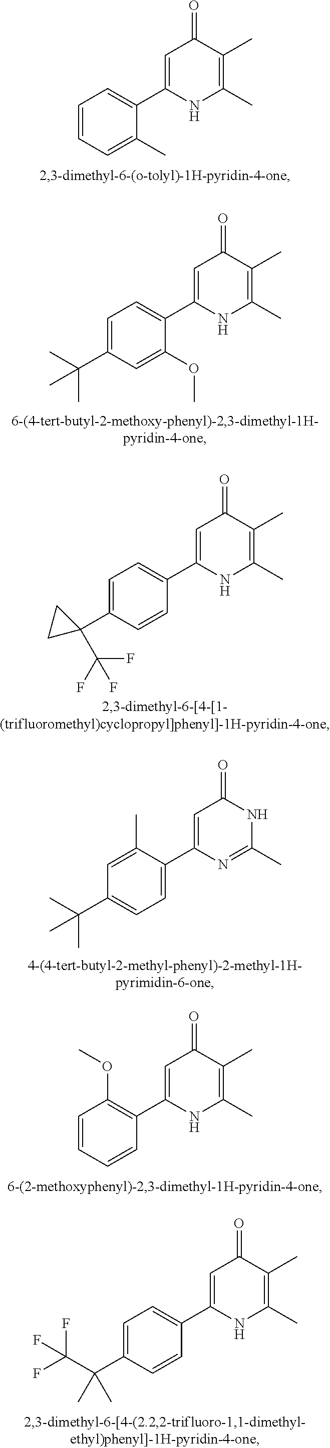 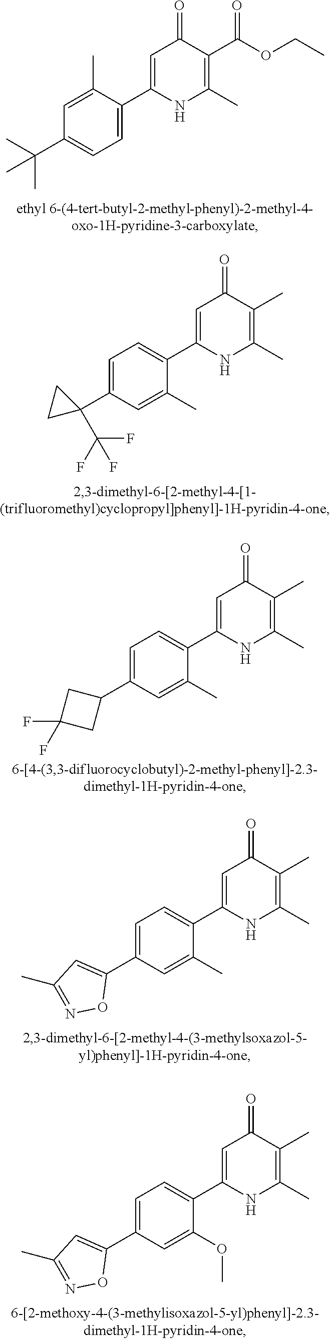 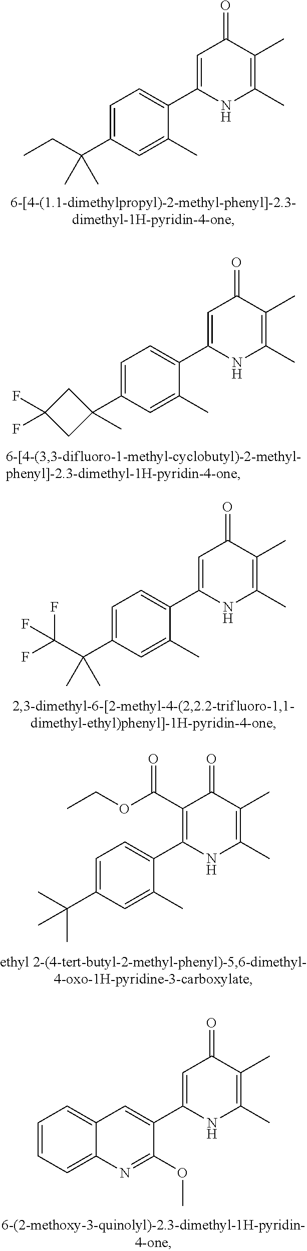 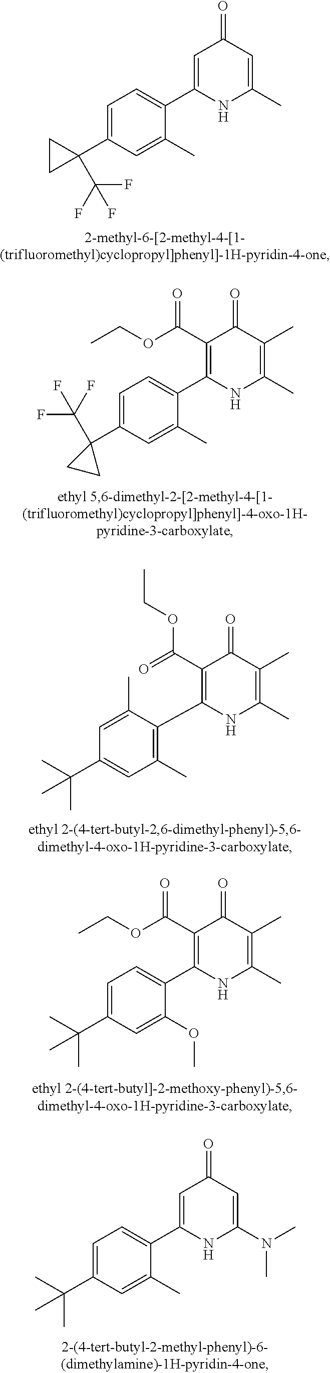 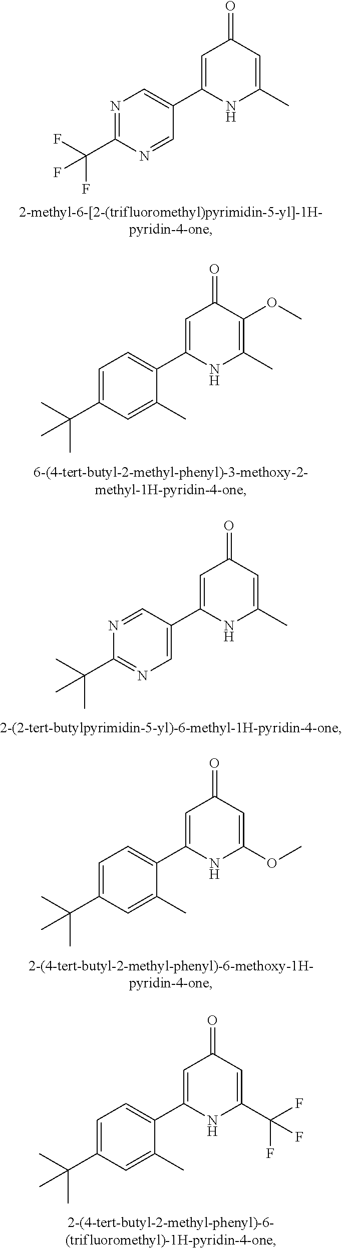 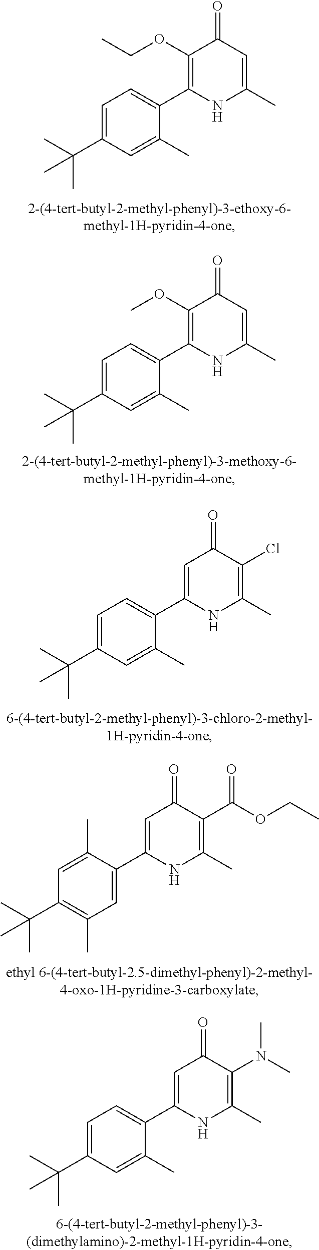 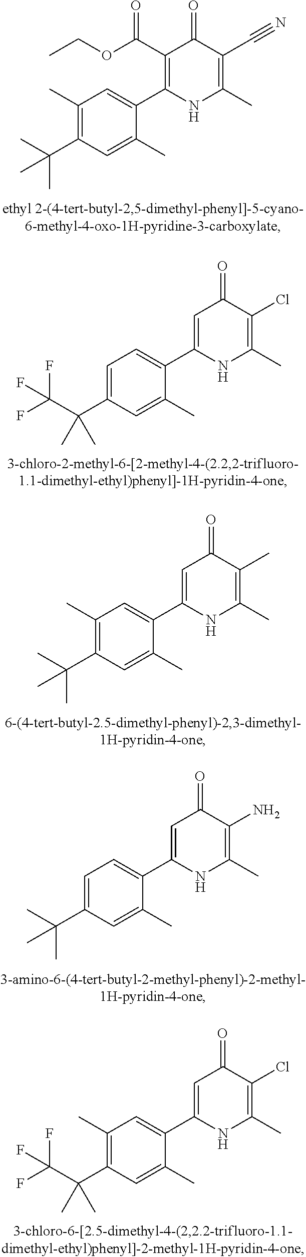 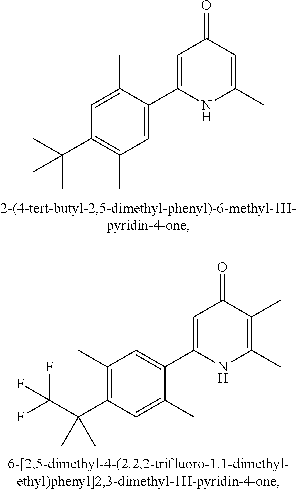 [SMILES: Cc1cc(=O)cc(-c2cc(C)c(C(C)(C)C)cc2C)[nH]1 ; Cc1cc(C(C)(C)C(F)(F)F)c(C)cc1-c1cc(=O)c(C)c(C)[nH]1] 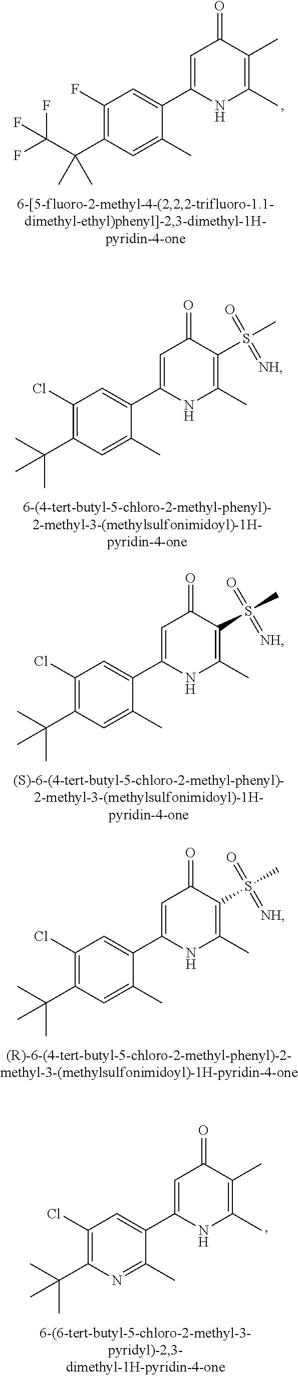 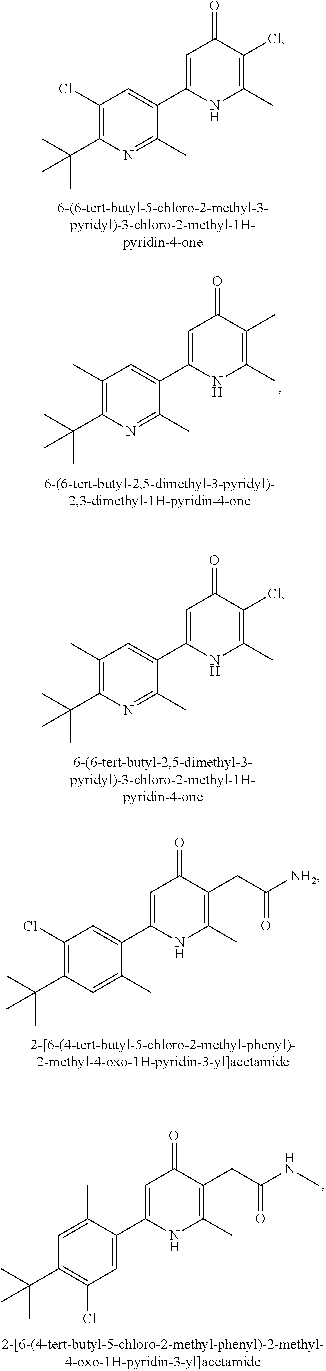 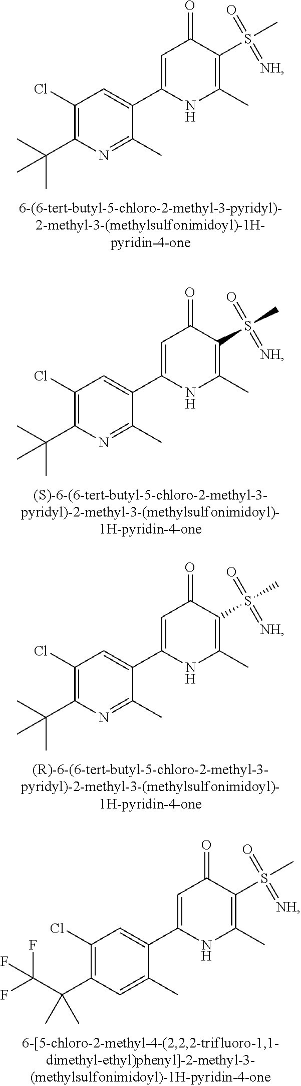 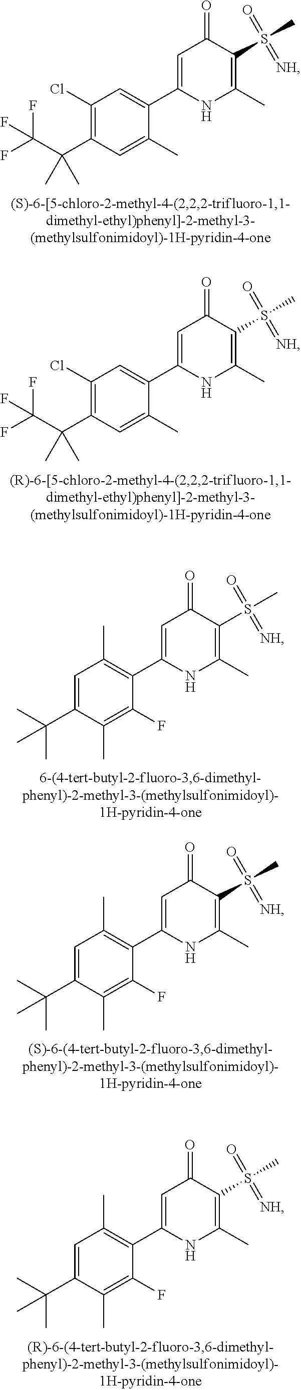 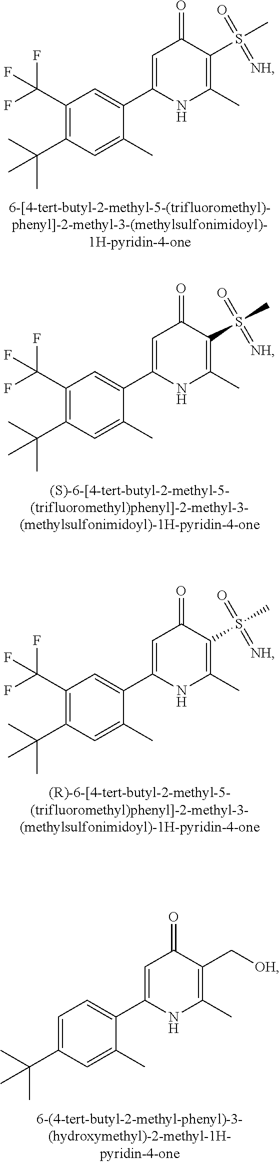 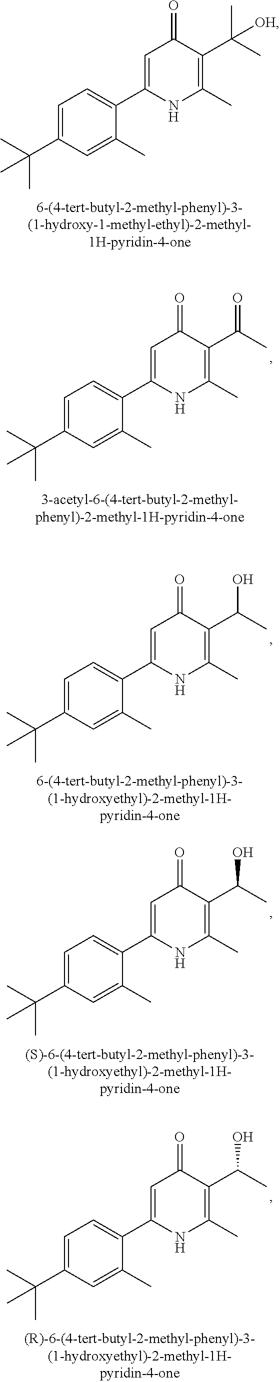 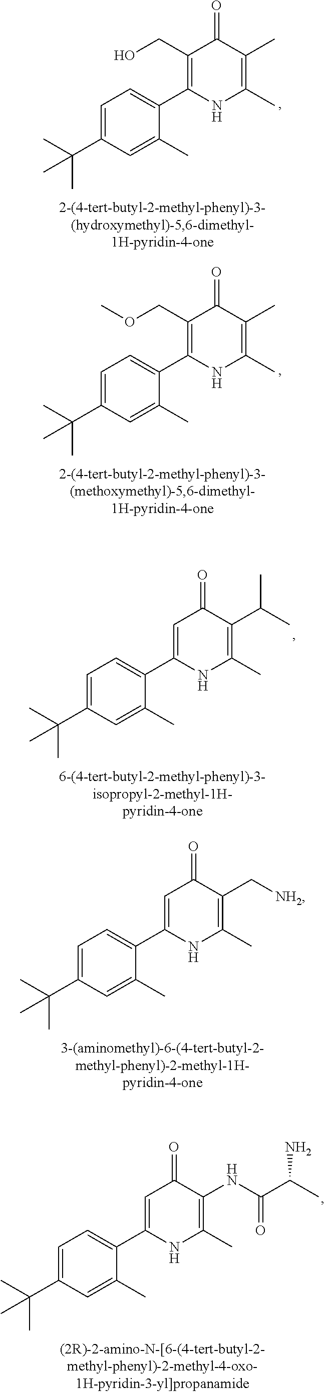 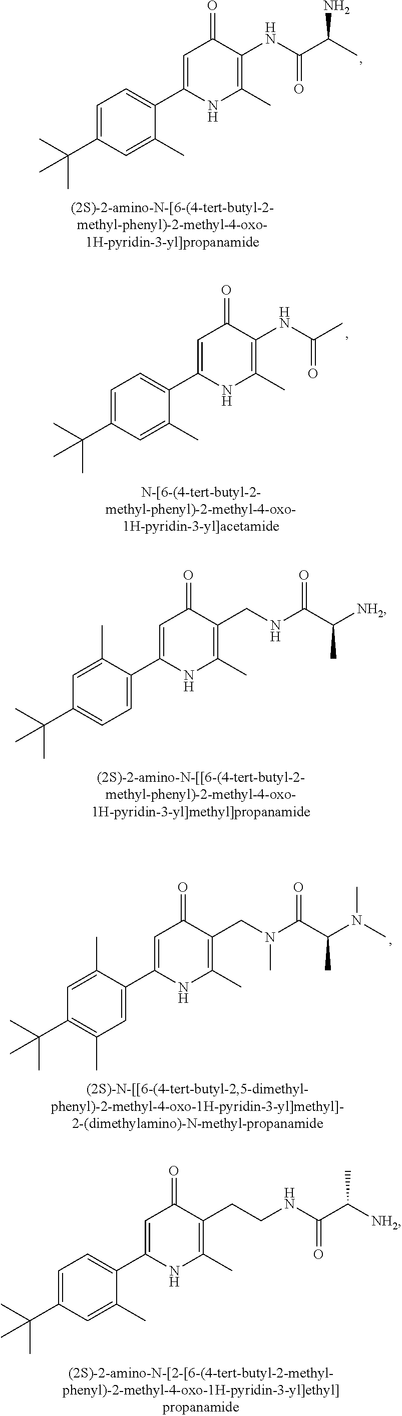 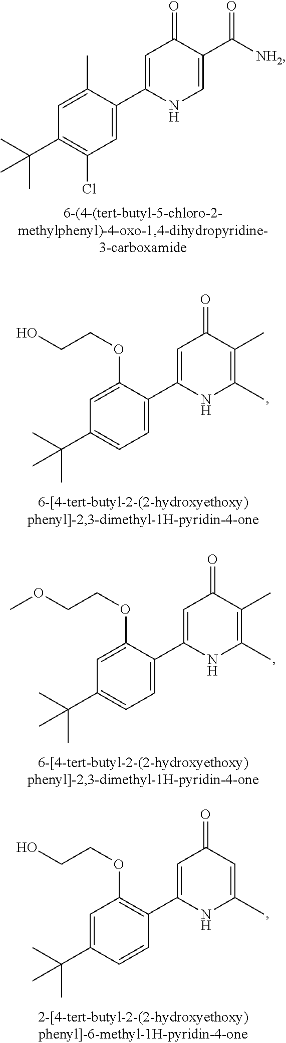 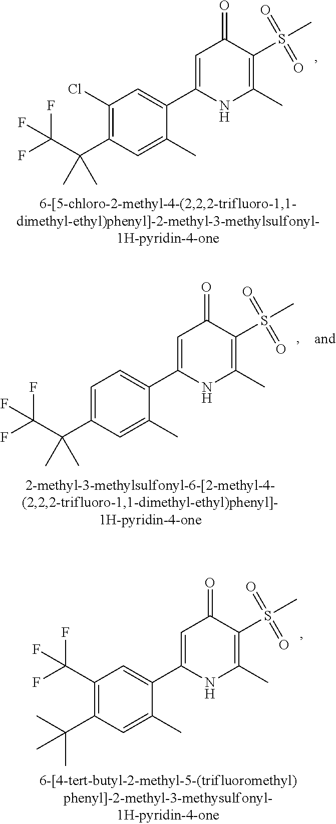 [SMILES: Cc1cc(C(C)(C)C(F)(F)F)c(Cl)cc1-c1cc(=O)c(S(C)(=O)=O)c(C)[nH]1 ; Cc1cc(C(C)(C)C(F)(F)F)ccc1-c1cc(=O)c(S(C)(=O)=O)c(C)[nH]1 ; Cc1cc(C(C)(C)C)c(C(F)(F)F)cc1-c1cc(=O)c(S(C)(=O)=O)c(C)[nH]1]
or a pharmaceutically acceptable salt thereof.

81. A compound selected from 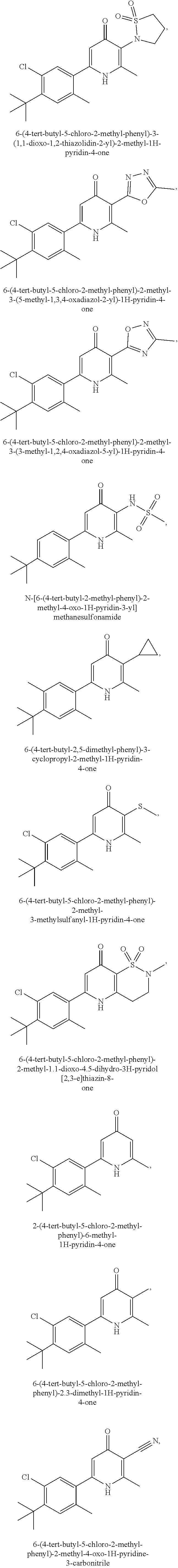 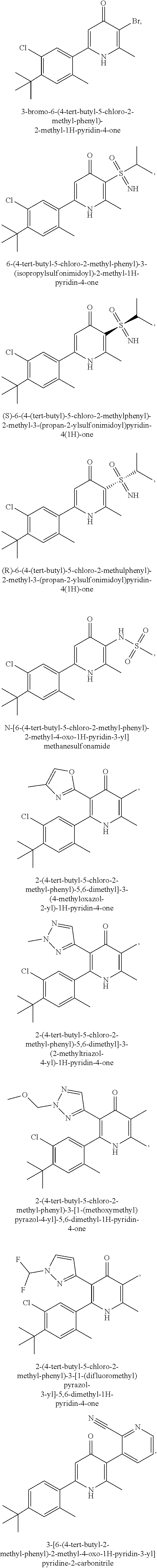 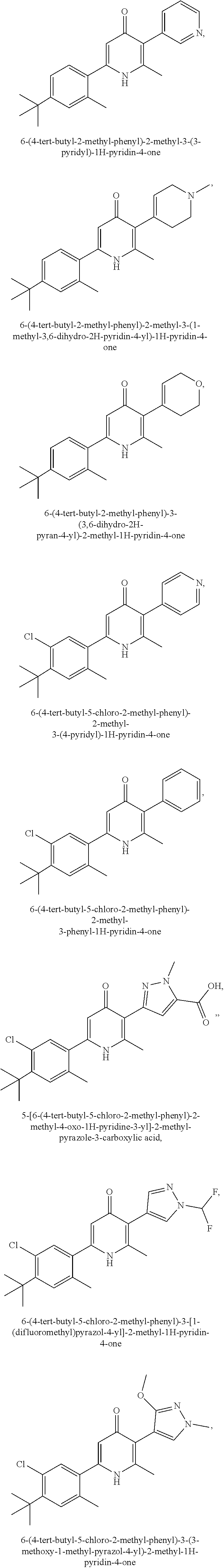 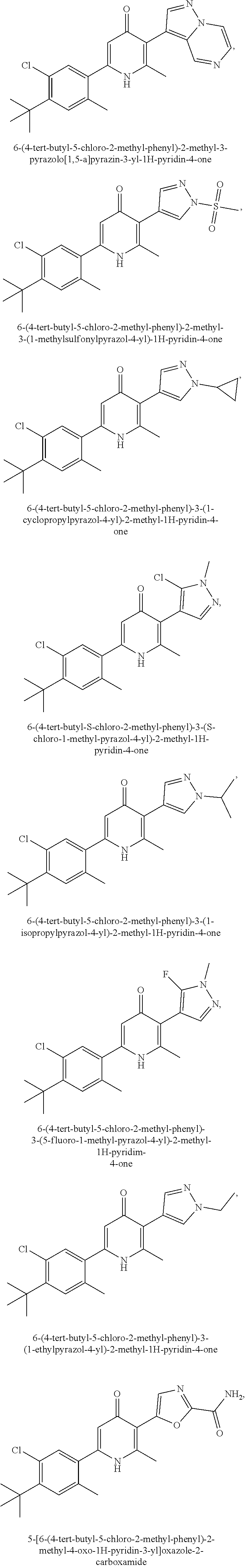 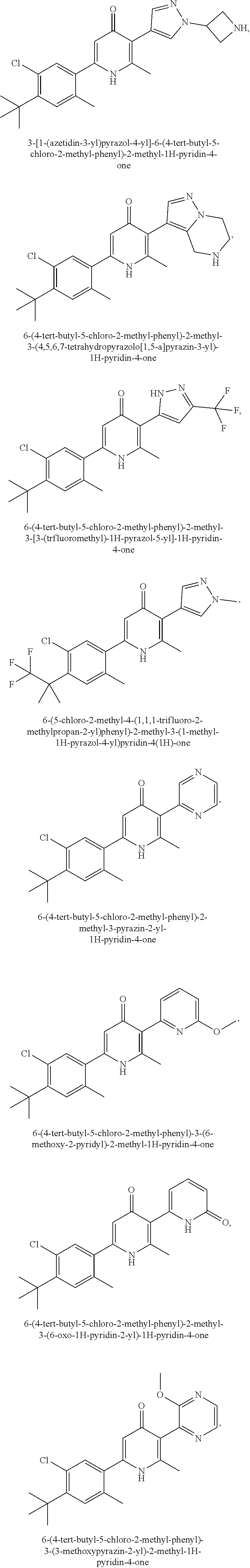 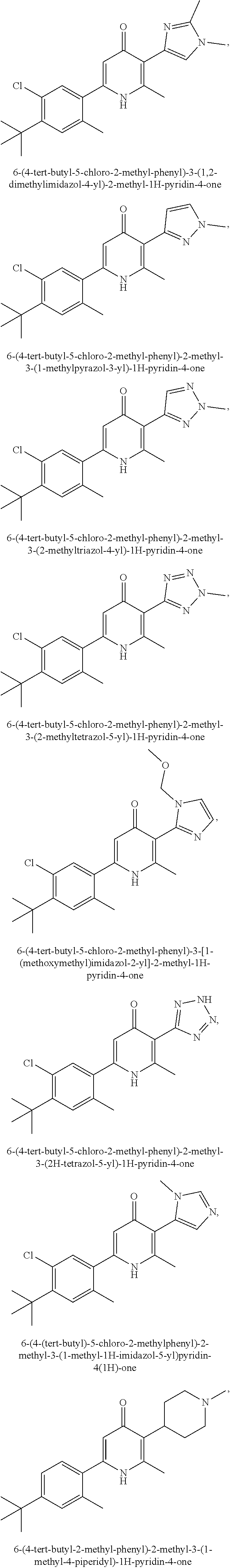 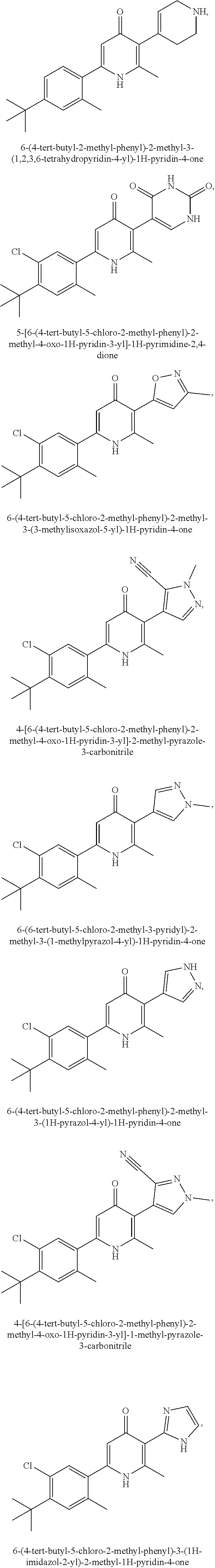 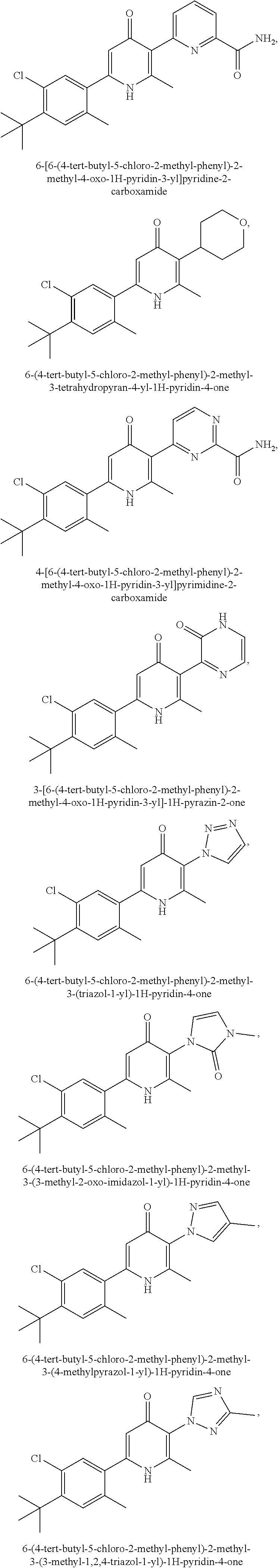 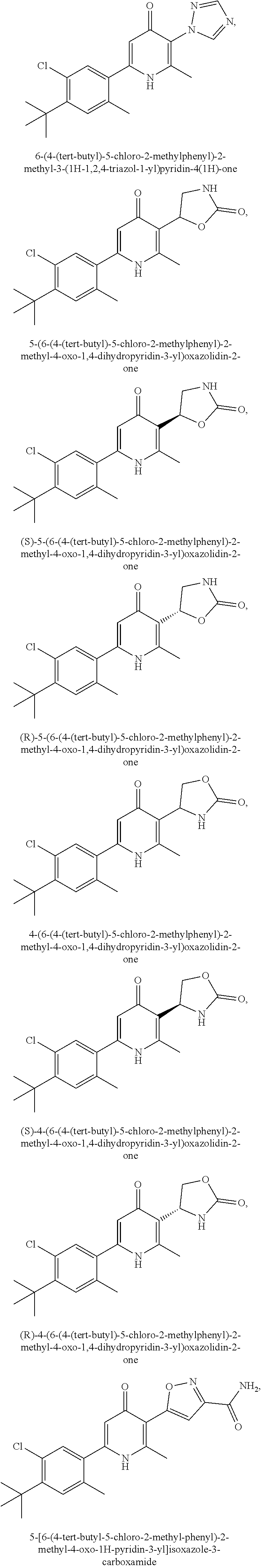 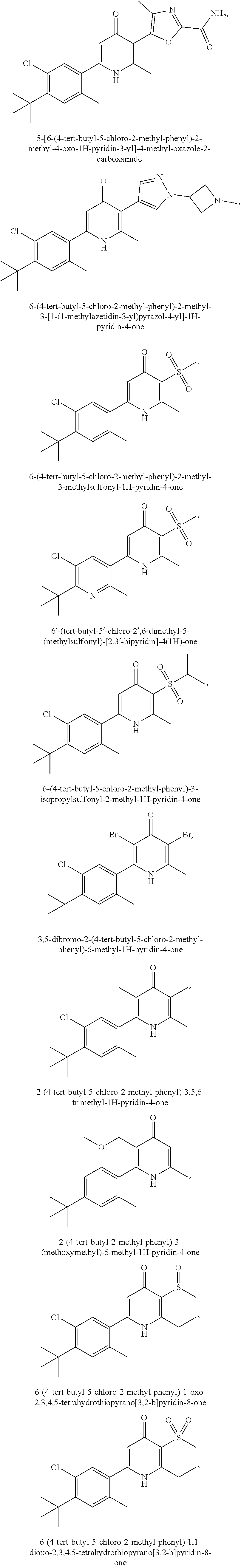 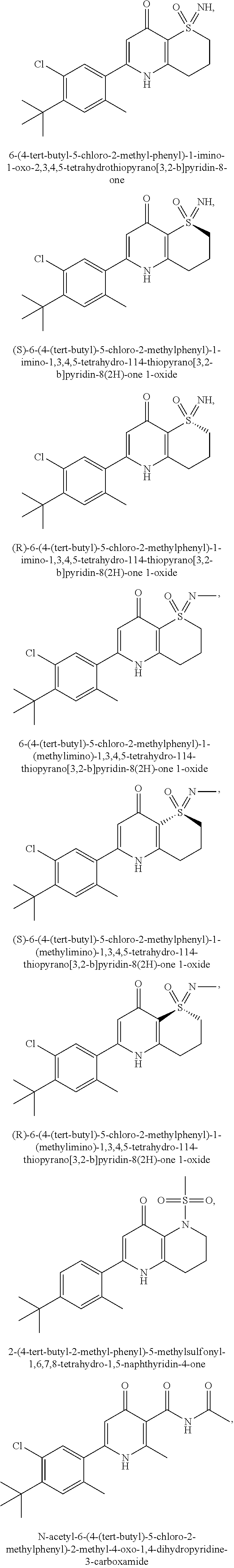 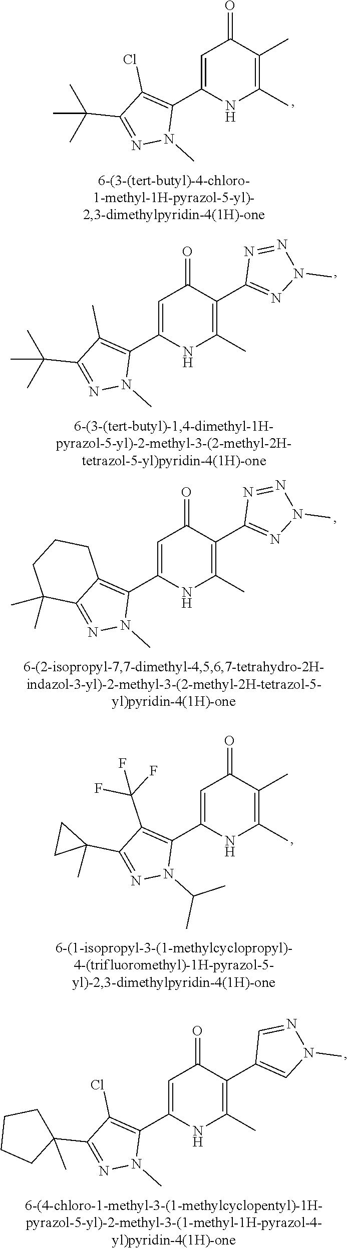 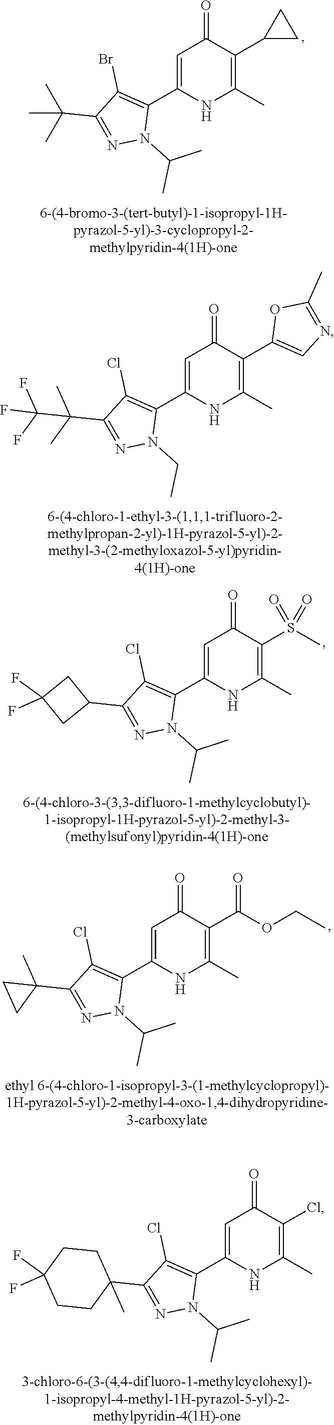 [SMILES: Cc1[nH]c(-c2c(Br)c(C(C)(C)C)nn2C(C)C)cc(=O)c1C1CC1 ; CCn1nc(C(C)(C)C(F)(F)F)c(Cl)c1-c1cc(=O)c(-c2cnc(C)o2)c(C)[nH]1 ; Cc1[nH]c(-c2c(Cl)c(C3CC(F)(F)C3)nn2C(C)C)cc(=O)c1S(C)(=O)=O ; CCOC(=O)c1c(C)[nH]c(-c2c(Cl)c(C3(C)CC3)nn2C(C)C)cc1=O ; Cc1[nH]c(-c2c(Cl)c(C3(C)CCC(F)(F)CC3)nn2C(C)C)cc(=O)c1Cl] 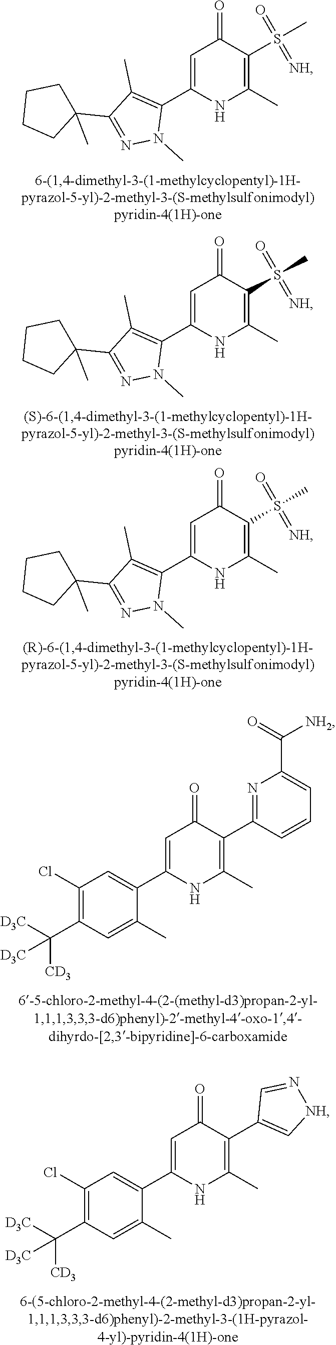 [SMILES: Cc1[nH]c(-c2c(C)c(C3(C)CCCC3)nn2C)cc(=O)c1S(C)(=N)=O ; Cc1[nH]c(-c2c(C)c(C3(C)CCCC3)nn2C)cc(=O)c1[S@@](C)(=N)=O ; Cc1[nH]c(-c2c(C)c(C3(C)CCCC3)nn2C)cc(=O)c1[S@](C)(=N)=O ; [2H]C([2H])([2H])C(c1cc(C)c(-c2cc(=O)c(-c3cccc(C(N)=O)n3)c(C)[nH]2)cc1Cl)(C([2H])([2H])[2H])C([2H])([2H])[2H] ; [2H]C([2H])([2H])C(c1cc(C)c(-c2cc(=O)c(-c3cn[nH]c3)c(C)[nH]2)cc1Cl)(C([2H])([2H])[2H])C([2H])([2H])[2H]] 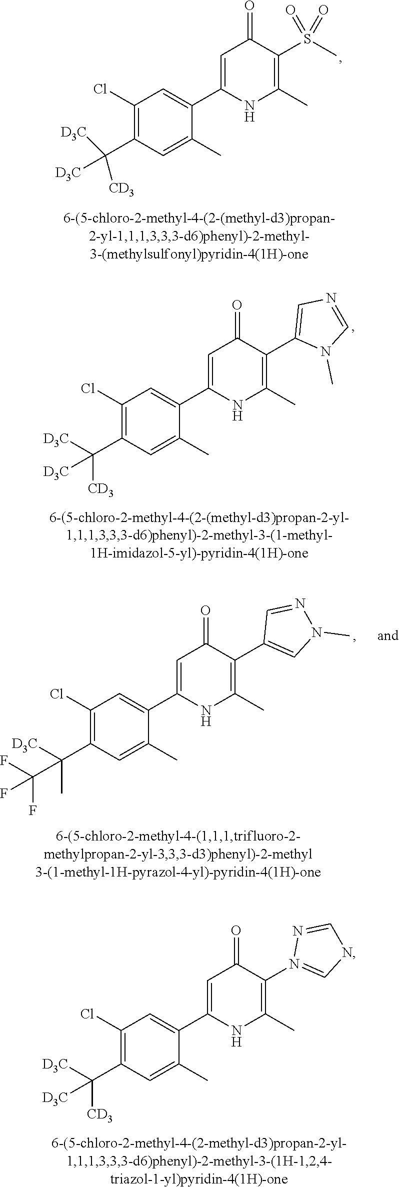
or a pharmaceutically acceptable salt thereof.

82. The compound of claim 1, wherein the compound is selected from: 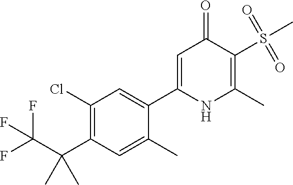 [SMILES: Cc1cc(C(C)(C)C(F)(F)F)c(Cl)cc1-c1cc(=O)c(S(C)(=O)=O)c(C)[nH]1]
6-[5-chloro-2-methyl-4-(2,2,2-trifluoro-1,1-dimethyl-ethyl)phenyl]-2-methyl-3-methylsulfonyl-1H-pyridin-4-one; 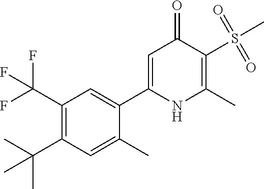 [SMILES: Cc1cc(C(C)(C)C)c(C(F)(F)F)cc1-c1cc(=O)c(S(C)(=O)=O)c(C)[nH]1]
6-[4-tert-butyl-2-methyl-5-(trifluoromethyl)phenyl]-2-methyl-3-methylsulfonyl-1H-pyridin-4-one; 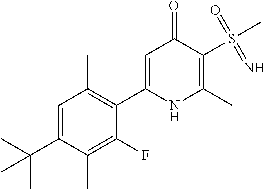 [SMILES: Cc1cc(C(C)(C)C)c(C)c(F)c1-c1cc(=O)c(S(C)(=N)=O)c(C)[nH]1]
6-(4-tert-butyl-2-fluoro-3,6-dimethyl-phenyl)-2-methyl-3-(methylsulfonimidoyl)-1H-pyridin-4-one; 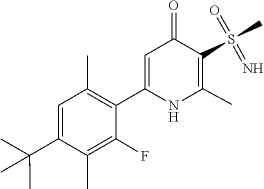 [SMILES: Cc1cc(C(C)(C)C)c(C)c(F)c1-c1cc(=O)c([S@@](C)(=N)=O)c(C)[nH]1]
(S)-6-(4-tert-butyl-2-fluoro-3,6-dimethyl-phenyl)-2-methyl-3-(methylsulfonimidoyl)-1H-pyridin-4-one; 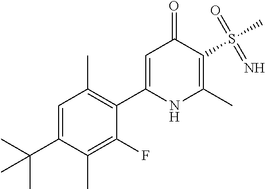 [SMILES: Cc1cc(C(C)(C)C)c(C)c(F)c1-c1cc(=O)c([S@](C)(=N)=O)c(C)[nH]1]
(R)-6-(4-tert-butyl-2-fluoro-3,6-dimethyl-phenyl)-2-methyl-3-(methylsulfonimidoyl)-1H-pyridin-4-one; 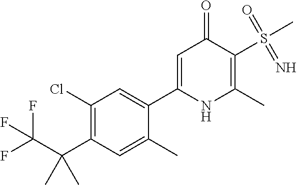 [SMILES: Cc1cc(C(C)(C)C(F)(F)F)c(Cl)cc1-c1cc(=O)c(S(C)(=N)=O)c(C)[nH]1]
6-[5-chloro-2-methyl-4-(2,2,2-trifluoro-1,1-dimethyl-ethyl)phenyl]-2-methyl-3-(methylsulfonimidoyl)-1H-pyridin-4-one; 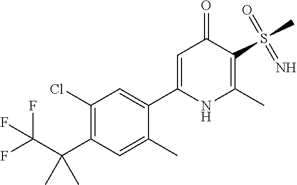 [SMILES: Cc1cc(C(C)(C)C(F)(F)F)c(Cl)cc1-c1cc(=O)c([S@@](C)(=N)=O)c(C)[nH]1]
(S)-6-[5-chloro-2-methyl-4-(2,2,2-trifluoro-1,1-dimethyl-ethyl)phenyl]-2-methyl-3-(methylsulfonimidoyl)-1H-pyridin-4-one; 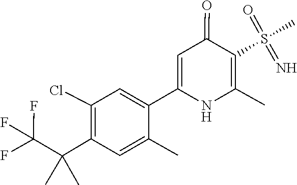 [SMILES: Cc1cc(C(C)(C)C(F)(F)F)c(Cl)cc1-c1cc(=O)c([S@](C)(=N)=O)c(C)[nH]1]
(R)-6-[5-chloro-2-methyl-4-(2,2,2-trifluoro-1,1-dimethyl-ethyl)phenyl]-2-methyl-3-(methylsulfonimidoyl)-1H-pyridin-4-one; 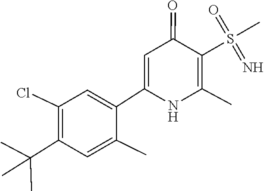 [SMILES: Cc1cc(C(C)(C)C)c(Cl)cc1-c1cc(=O)c(S(C)(=N)=O)c(C)[nH]1]
6-(4-tert-butyl-5-chloro-2-methyl-phenyl)-2-methyl-3-(methylsulfonimidoyl)-1H-pyridin-4-one; 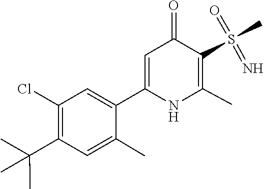 [SMILES: Cc1cc(C(C)(C)C)c(Cl)cc1-c1cc(=O)c([S@@](C)(=N)=O)c(C)[nH]1]
(S)-6-(4-tert-butyl-5-chloro-2-methyl-phenyl)-2-methyl-3-(methylsulfonimidoyl)-1H-pyridin-4-one; 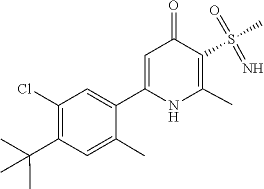 [SMILES: Cc1cc(C(C)(C)C)c(Cl)cc1-c1cc(=O)c([S@](C)(=N)=O)c(C)[nH]1]
(R)-6-(4-tert-butyl-5-chloro-2-methyl-phenyl)-2-methyl-3-(methylsulfonimidoyl)-1H-pyridin-4-one; 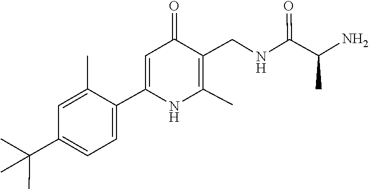 [SMILES: Cc1cc(C(C)(C)C)ccc1-c1cc(=O)c(CNC(=O)[C@H](C)N)c(C)[nH]1]
(2S)-2-amino-N-[[6-(4-tert-butyl-2-methyl-phenyl)-2-methyl-4-oxo-1H-pyridin-3-yl]methyl]propanamide; 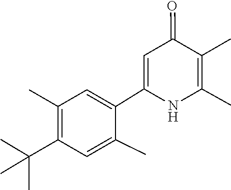 [SMILES: Cc1cc(C(C)(C)C)c(C)cc1-c1cc(=O)c(C)c(C)[nH]1]
6-(4-tert-butyl-2,5-dimethyl-phenyl)-2,3-dimethyl-1H-pyridin-4-one; 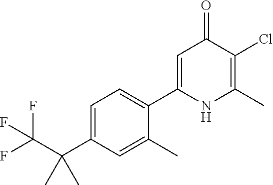 [SMILES: Cc1cc(C(C)(C)C(F)(F)F)ccc1-c1cc(=O)c(Cl)c(C)[nH]1]
3-chloro-2-methyl-6-[2-methyl-4-(2,2,2-trifluoro-1,1-dimethyl-ethyl)phenyl]-1H-pyridin-4-one; 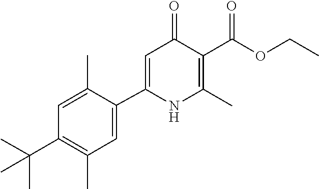 [SMILES: CCOC(=O)c1c(C)[nH]c(-c2cc(C)c(C(C)(C)C)cc2C)cc1=O]
ethyl 6-(4-tert-butyl-2,5-dimethyl-phenyl)-2-methyl-4-oxo-1H-pyridine-3-carboxylate; 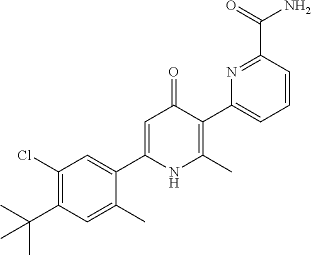 [SMILES: Cc1cc(C(C)(C)C)c(Cl)cc1-c1cc(=O)c(-c2cccc(C(N)=O)n2)c(C)[nH]1]
6-[6-(4-tert-butyl-5-chloro-2-methyl-phenyl)-2-methyl-4-oxo-H-pyridin-3-yl]pyridine-2-carboxamide; 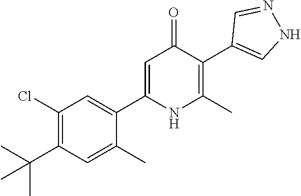 [SMILES: Cc1cc(C(C)(C)C)c(Cl)cc1-c1cc(=O)c(-c2cn[nH]c2)c(C)[nH]1]
6-(4-tert-butyl-5-chloro-2-methyl-phenyl)-2-methyl-3-(1H-pyrazol-4-yl)-1H-pyridin-4-one; 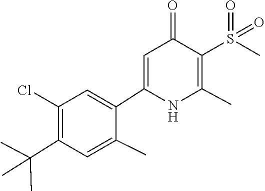 [SMILES: Cc1cc(C(C)(C)C)c(Cl)cc1-c1cc(=O)c(S(C)(=O)=O)c(C)[nH]1]
6-(4-tert-butyl-5-chloro-2-methyl-phenyl)-2-methyl-3-methylsulfonyl-1H-pyridin-4-one; 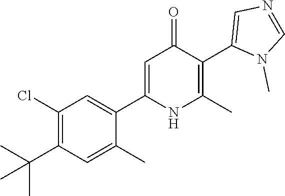 [SMILES: Cc1cc(C(C)(C)C)c(Cl)cc1-c1cc(=O)c(-c2cncn2C)c(C)[nH]1]
6-(4-tert-butyl-5-chloro-2-methylphenyl)-2-methyl-3-(1-methyl-1H-imidazol-5-yl)pyridin-4(1H)-one; 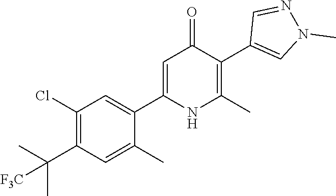 [SMILES: Cc1cc(C(C)(C)C(F)(F)F)c(Cl)cc1-c1cc(=O)c(-c2cnn(C)c2)c(C)[nH]1]
6-(5-chloro-2-methyl-4-(1,1,1-trifluoro-2-methylpropan-2-yl)phenyl)-2-methyl-3-(1-methyl-1H-pyrazol-4-yl)pyridin-4(1H)-one; and 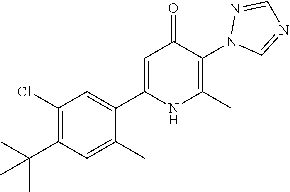 [SMILES: Cc1cc(C(C)(C)C)c(Cl)cc1-c1cc(=O)c(-n2cncn2)c(C)[nH]1]
6-(4-(tert-butyl)-5-chloro-2-methylphenyl)-2-methyl-3-(1H-1,2,4-triazol-1-yl)pyridin-4(1H)-one, or a pharmaceutically acceptable salt thereof.

83. The compound of claim 1 in a non-salt form.

84. (canceled)

85. A pharmaceutical composition comprising
the compound of claim 1, or a pharmaceutically acceptable salt thereof,
and one or more pharmaceutically acceptable carriers or vehicles.

86. A method of inhibiting a voltage-gated sodium channel in a subject comprising administering to the subject
the compound of claim 1, or a pharmaceutically acceptable salt thereof.

87. The method of claim 86, wherein the voltage-gated sodium channel is Na_{V}1.8.

88. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of
the compound of claim 1, or a pharmaceutically acceptable salt thereof.

89. The method of claim 88, wherein
(i) the method comprises treating or lessening the severity in the subject of chronic pain; or
(ii) the method comprises treating or lessening the severity in the subject of neuropathic pain; or
(iii) the method comprises treating or lessening the severity in the subject of musculoskeletal pain; or
(iv) the method comprises treating or lessening the severity in the subject of acute pain.

90-92. (canceled)

93. The method of claim 88, wherein said subject is treated with one or more additional therapeutic agents administered concurrently with, prior to, or subsequent to treatment with the compound, pharmaceutically acceptable salt, or pharmaceutical composition.

94-95. (canceled)
